SEQUENCE LISTING (174 sequences)

SEQ ID NO: 1 - protein, 117 aa, synthetic construct
QVQLQQWGAGLLKPSETLSLTCAVYGGSFSGYYWSWIRQPPGKGLEWIGEIDHSGSTNYNPSLKSRVTISVDTSKNQFSLKLSSVTAADTAVYYCARARGPWSFDPWGQGTLVTVSS

SEQ ID NO: 2 - protein, 9 aa, synthetic construct
GSFSGYYWS

SEQ ID NO: 3 - protein, 16 aa, synthetic construct
EIDHSGSTNYNPSLKS

SEQ ID NO: 4 - protein, 11 aa, synthetic construct
ARARGPWSFDP

SEQ ID NO: 5 - protein, 107 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQYNSYPITFGGGTKVEIK

SEQ ID NO: 6 - protein, 108 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPITFGGGTKVEIK

SEQ ID NO: 7 - protein, 106 aa, synthetic construct
DIQMTQSPSTLSASVGDRVTITCRASQSIGSWLAWYQQKPGKAPKLLIYKASSLESGVPSRFSGSGSGTEFTLTISSLQPDDFATYYCQQYHSFYTFGGGTKVEIK

SEQ ID NO: 8 - protein, 106 aa, synthetic construct
DIQMTQSPSTLSASVGDRVTITCRASQSIGSWLAWYQQKPGKAPKLLIYKASSLESGVPSRFSGSGSGTEFTLTISSLQPDDFATYYCQQSNSYYTFGGGTKVEIK

SEQ ID NO: 9 - protein, 106 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQYNSYPTFGGGTKVEIK

SEQ ID NO: 10 - protein, 117 aa, synthetic construct
QVQLQQWGAGLLKPSETLSLTCAVYGGSFSGYYWSWIRQPPGKGLEWIGEIDHSGSTNYNPSLKSRVTISVDTSKNQFSLKLSSVTAADTAVYYCARARGPWGFDPWGQGTLVTVSS

SEQ ID NO: 11 - protein, 107 aa, synthetic construct
ELQMTQSPSSLSASVGDRVTITCRTSQSISSYLNWYQQKPGQPPKLLIYWASTRESGVPDRFSGSGSGTDFTLTISSLQPEDSATYYCQQSYDIPYTFGQGTKLEIK

SEQ ID NO: 12 - protein, 107 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQYGSFPITFGGGTKVEIK

SEQ ID NO: 13 - protein, 107 aa, synthetic construct
DIQMTQSPSTLSASVGDRVTITCRASQSISSWLAWYQQKPGKAPKLLIYKASSLESGVPSRFSGSGSGTDFTLTISSLQPDDFATYYCQQSKEVPWTFGQGTKVEIK

SEQ ID NO: 14 - protein, 106 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQYNSFPTFGGGTKVEIK

SEQ ID NO: 15 - protein, 106 aa, synthetic construct
DIQMTQSPSTLSASVGDRVTITCRASQSIGSWLAWYQQKPGKAPKLLIYKASSLESGVPSRFSGSGSGTEFTLTISSLQPDDFATYYCQQYDIYPTFGGGTKVEIK

SEQ ID NO: 16 - protein, 106 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQYDSYPTFGGGTKVEIK

SEQ ID NO: 17 - protein, 106 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQYGSFPTFGGGTKVEIK

SEQ ID NO: 18 - protein, 106 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQYQSFPTFGGGTKVEIK

SEQ ID NO: 19 - protein, 106 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQYSSFSTFGGGTKVEIK

SEQ ID NO: 20 - protein, 106 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQYESYSTFGGGTKVEIK

SEQ ID NO: 21 - protein, 106 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQYDSFITFGGGTKVEIK

SEQ ID NO: 22 - protein, 106 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQYQSYPTFGGGTKVEIK

SEQ ID NO: 23 - protein, 106 aa, synthetic construct
DIQMTQSPSTLSASVGDRVTITCRASQSIGSWLAWYQQKPGKAPKLLIYKASSLESGVPSRFSGSGSGTEFTLTISSLQPDDFATYYCQQYHSFPTFGGGTKVEIK

SEQ ID NO: 24 - protein, 107 aa, synthetic construct
DIQMTQSPSTLSASVGDRVTITCRASQSIGSWLAWYQQKPGKAPKLLIYKASSLESGVPSRFSGSGSGTEFTLTISSLQPDDFATYYCQQYELYSYTFGGGTKVEIK

SEQ ID NO: 25 - protein, 106 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQYDTFITFGGGTKVEIK

SEQ ID NO: 26 - protein, 125 aa, synthetic construct
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARGDSSIRHAYYYYGMDVWGQGTTVTVSS

SEQ ID NO: 27 - protein, 9 aa, synthetic construct
GTFSSYAIS

SEQ ID NO: 28 - protein, 5 aa, synthetic construct
SYAIS

SEQ ID NO: 29 - protein, 17 aa, synthetic construct
GIIPIFGTANYAQKFQG

SEQ ID NO: 30 - protein, 18 aa, synthetic construct
ARGDSSIRHAYYYYGMDV

SEQ ID NO: 31 - protein, 16 aa, synthetic construct
GDSSIRHAYYYYGMDV

SEQ ID NO: 32 - protein, 113 aa, synthetic construct
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYYSTPITFGGGTKVEIK

SEQ ID NO: 33 - protein, 17 aa, synthetic construct
KSSQSVLYSSNNKNYLA

SEQ ID NO: 34 - protein, 7 aa, synthetic construct
WASTRES

SEQ ID NO: 35 - protein, 9 aa, synthetic construct
QQYYSTPIT

SEQ ID NO: 36 - protein, 121 aa, synthetic construct
QLQLQESGPGLVKPSETLSLTCTVSGGSISSSSYYWGWIRQPPGKGLEWIGSIYYSGSTYYNPSLKSRVTISVDTSKNQFSLKLSSVTAADTAVYYCARGSDRFHPYFDYWGQGTLVTVSS

SEQ ID NO: 37 - protein, 11 aa, synthetic construct
GSISSSSYYWG

SEQ ID NO: 38 - protein, 7 aa, synthetic construct
SSSYYWG

SEQ ID NO: 39 - protein, 16 aa, synthetic construct
SIYYSGSTYYNPSLKS

SEQ ID NO: 40 - protein, 13 aa, synthetic construct
ARGSDRFHPYFDY

SEQ ID NO: 41 - protein, 11 aa, synthetic construct
GSDRFHPYFDY

SEQ ID NO: 42 - protein, 107 aa, synthetic construct
EIVLTQSPATLSLSPGERATLSCRASQSVSRYLAWYQQKPGQAPRLLIYDASNRATGIPARFSGSGSGTDFTLTISSLEPEDFAVYYCQQFDTWPPTFGGGTKVEIK

SEQ ID NO: 43 - protein, 11 aa, synthetic construct
RASQSVSRYLA

SEQ ID NO: 44 - protein, 7 aa, synthetic construct
DASNRAT

SEQ ID NO: 45 - protein, 9 aa, synthetic construct
QQFDTWPPT

SEQ ID NO: 46 - protein, 106 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCEQYDSYPTFGGGTKVEIK

SEQ ID NO: 47 - protein, 126 aa, synthetic construct
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARRGRKASGSFYYYYGMDVWGQGTTVTVSS

SEQ ID NO: 48 - protein, 19 aa, synthetic construct
ARRGRKASGSFYYYYGMDV

SEQ ID NO: 49 - protein, 113 aa, synthetic construct [sequence omitted]

SEQ ID NO: 50 - protein, 17 aa, synthetic construct
ESSQSLLNSGNQKNYLT

SEQ ID NO: 51 - protein, 9 aa, synthetic construct
QNDYSYPYT

SEQ ID NO: 52 - protein, 126 aa, synthetic construct
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARGAPNYGDTTHDYYYMDVWGKGTTVTVSS

SEQ ID NO: 53 - protein, 9 aa, synthetic construct
YTFTSYYMH

SEQ ID NO: 54 - protein, 5 aa, synthetic construct
SYYMH

SEQ ID NO: 55 - protein, 17 aa, synthetic construct
IINPSGGSTSYAQKFQG

SEQ ID NO: 56 - protein, 19 aa, synthetic construct
ARGAPNYGDTTHDYYYMDV

SEQ ID NO: 57 - protein, 17 aa, synthetic construct
GAPNYGDTTHDYYYMDV

SEQ ID NO: 58 - protein, 107 aa, synthetic construct
EIVMTQSPATLSVSPGERATLSCRASQSVSSNLAWYQQKPGQAPRLLIYGASTRATGIPARFSGSGSGTEFTLTISSLQSEDFAVYYCQQYDDWPFTFGGGTKVEIK

SEQ ID NO: 59 - protein, 11 aa, synthetic construct
RASQSVSSNLA

SEQ ID NO: 60 - protein, 7 aa, synthetic construct
GASTRAT

SEQ ID NO: 61 - protein, 9 aa, synthetic construct
QQYDDWPFT

SEQ ID NO: 62 - protein, 124 aa, synthetic construct
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCARDTGEYYDTDDHGMDVWGQGTTVTVSS

SEQ ID NO: 63 - protein, 9 aa, synthetic construct
YTFTGYYMH

SEQ ID NO: 64 - protein, 5 aa, synthetic construct
GYYMH

SEQ ID NO: 65 - protein, 17 aa, synthetic construct
WINPNSGGTNYAQKFQG

SEQ ID NO: 66 - protein, 17 aa, synthetic construct
ARDTGEYYDTDDHGMDV

SEQ ID NO: 67 - protein, 15 aa, synthetic construct
DTGEYYDTDDHGMDV

SEQ ID NO: 68 - protein, 107 aa, synthetic construct
EIVLTQSPGTLSLSPGERATLSCRASQSVSSNLAWYQQKPGQAPRLLIYGASTRATGIPARFSGSGSGTEFTLTISSLQSEDFAVYYCQQDDYWPPTFGGGTKVEIK

SEQ ID NO: 69 - protein, 9 aa, synthetic construct
QQDDYWPPT

SEQ ID NO: 70 - protein, 121 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKDGGYYDSGAGDYWGQGTLVTVSS

SEQ ID NO: 71 - protein, 9 aa, synthetic construct
FTFSSYAMS

SEQ ID NO: 72 - protein, 17 aa, synthetic construct
AISGSGGSTYYADSVKG

SEQ ID NO: 73 - protein, 14 aa, synthetic construct
AKDGGYYDSGAGDY

SEQ ID NO: 74 - protein, 12 aa, synthetic construct
DGGYYDSGAGDY

SEQ ID NO: 75 - protein, 107 aa, synthetic construct
DIQMTQSPSSVSASVGDRVTITCRASQGIDSWLAWYQQKPGKAPKLLIYAASSLQSGVPSRFSGSGSGTDFTLTISSLQPEDFATYYCQQGVSYPRTFGGGTKVEIK

SEQ ID NO: 76 - protein, 11 aa, synthetic construct
RASQGIDSWLA

SEQ ID NO: 77 - protein, 7 aa, synthetic construct
AASSLQS

SEQ ID NO: 78 - protein, 9 aa, synthetic construct
QQGVSYPRT

SEQ ID NO: 79 - protein, 122 aa, synthetic construct
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGAPMGAAAGWFDPWGQGTLVTVSS

SEQ ID NO: 80 - protein, 9 aa, synthetic construct
FTFSSYSMN

SEQ ID NO: 81 - protein, 5 aa, synthetic construct
SYSMN

SEQ ID NO: 82 - protein, 17 aa, synthetic construct
SISSSSSYIYYADSVKG

SEQ ID NO: 83 - protein, 15 aa, synthetic construct
ARGAPMGAAAGWFDP

SEQ ID NO: 84 - protein, 13 aa, synthetic construct
GAPMGAAAGWFDP

SEQ ID NO: 85 - protein, 107 aa, synthetic construct
DIQMTQSPSSVSASVGDRVTITCRASQGISSWLAWYQQKPGKAPKLLIYAASSLQSGVPSRFSGSGSGTDFTLTISSLQPEDFATYYCQQGVSFPRTFGGGTKVEIK

SEQ ID NO: 86 - protein, 11 aa, synthetic construct
RASQGISSWLA

SEQ ID NO: 87 - protein, 9 aa, synthetic construct
QQGVSFPRT

SEQ ID NO: 88 - protein, 249 aa, synthetic construct
DIQMTQSPSSVSASVGDRVTITCRASQGISSWLAWYQQKPGKAPKLLIYAASSLQSGVPSRFSGSGSGTDFTLTISSLQPEDFATYYCQQGVSFPRTFGCGTKVEIKGGGGSGGGGSGGGGSGGGGSEVQLVESGGGLVKPGGSLRLSCAASGFTFSSYSMNWVRQAPGKCLEWVSSISSSSSYIYYADSVKGRFTISRDNAKNSLYLQMNSLRAEDTAVYYCARGAPMGAAAGWFDPWGQGTLVTVSS

SEQ ID NO: 89 - protein, 125 aa, synthetic construct
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCAREGAGFAYGMDYYYMDVWGKGTTVTVSS

SEQ ID NO: 90 - protein, 18 aa, synthetic construct
AREGAGFAYGMDYYYMDV

SEQ ID NO: 91 - protein, 16 aa, synthetic construct
EGAGFAYGMDYYYMDV

SEQ ID NO: 92 - protein, 107 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQSDNWPFTFGGGTKVEIK

SEQ ID NO: 93 - protein, 11 aa, synthetic construct
RASQSVSSYLA

SEQ ID NO: 94 - protein, 9 aa, synthetic construct
QQSDNWPFT

SEQ ID NO: 95 - protein, 122 aa, synthetic construct
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGAPIGAAAGWFDPWGQGTLVTVSS

SEQ ID NO: 96 - protein, 15 aa, synthetic construct
ARGAPIGAAAGWFDP

SEQ ID NO: 97 - protein, 13 aa, synthetic construct
GAPIGAAAGWFDP

SEQ ID NO: 98 - protein, 122 aa, synthetic construct
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGAPQGAAAGWFDPWGQGTLVTVSS

SEQ ID NO: 99 - protein, 15 aa, synthetic construct
ARGAPQGAAAGWFDP

SEQ ID NO: 100 - protein, 13 aa, synthetic construct
GAPQGAAAGWFDP

SEQ ID NO: 101 - protein, 122 aa, synthetic construct
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGAPLGAAAGWFDPWGQGTLVTVSS

SEQ ID NO: 102 - protein, 15 aa, synthetic construct
ARGAPLGAAAGWFDP

SEQ ID NO: 103 - protein, 13 aa, synthetic construct
GAPLGAAAGWFDP

SEQ ID NO: 104 - protein, 122 aa, synthetic construct
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGAPFGAAAGWFDPWGQGTLVTVSS

SEQ ID NO: 105 - protein, 15 aa, synthetic construct
ARGAPFGAAAGWFDP

SEQ ID NO: 106 - protein, 13 aa, synthetic construct
GAPFGAAAGWFDP

SEQ ID NO: 107 - protein, 122 aa, synthetic construct
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGAPVGAAAGWFDPWGQGTLVTVSS

SEQ ID NO: 108 - protein, 15 aa, synthetic construct
ARGAPVGAAAGWFDP

SEQ ID NO: 109 - protein, 13 aa, synthetic construct
GAPVGAAAGWFDP

SEQ ID NO: 110 - protein, 122 aa, synthetic construct
  VARIANT 102: note = X can be M, L, I, V, Q, or F
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGAPXGAAAGWFDPWGQGTLVTVSS

SEQ ID NO: 111 - protein, 15 aa, synthetic construct
  VARIANT 6: note = X can be M, L, I, V, Q, or F
ARGAPXGAAAGWFDP

SEQ ID NO: 112 - protein, 13 aa, synthetic construct
  VARIANT 4: note = X can be M, L, I, V, Q, or F
GAPXGAAAGWFDP

SEQ ID NO: 113 - protein, 121 aa, synthetic construct
QVQLVESGGGLVKPGGSLRLSCAASGFTFSSYGMHWVRQAPGKGLEWVAFIRYDGSNKYYADSVKGRFTISRDNSKNTLYLQMNSLRAEDTAVYYCAKDRGLGDGTYFDYWGQGTTVTVSS

SEQ ID NO: 114 - protein, 110 aa, synthetic construct
QSALTQPASVSGSPGQSITISCSGSSSNIGNNAVNWYQQLPGKAPKLLIYYDDLLPSGVSDRFSGSKSGTSAFLAISGLQSEDEADYYCAAWDDSLNGPVFGGGTKLTVL

SEQ ID NO: 115 - protein, 5 aa, synthetic construct
SYAMS

SEQ ID NO: 116 - protein, 115 aa, synthetic construct
QVHLQESGPGLVKPSETLSLTCTVSDDSISSYYWSWIRQPPGKGLEWIGHISYSGSANYNPSLKSRVTISVDTSKNQFSLKLSSVTAADTAVYYCANWDDAFNIWGQGTMVTVSS

SEQ ID NO: 117 - protein, 108 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIK

SEQ ID NO: 118 - protein, 226 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 119 - protein, 20 aa, synthetic construct
GGGGSGGGGSGGGGSGGGGS

SEQ ID NO: 120 - protein, 4 aa, synthetic construct
  REGION >4: note = The entire sequence of amino acids 1-4 can be repeated one or more times.
GSGS

SEQ ID NO: 121 - protein, 6 aa, synthetic construct
  REGION >6: note = The entire sequence of amino acids 1-6 can be repeated one or more times.
GGSGGS

SEQ ID NO: 122 - protein, 4 aa, synthetic construct
  REGION >4: note = The entire sequence of amino acids 1-4 can be repeated one or more times.
GGGS

SEQ ID NO: 123 - protein, 4 aa, synthetic construct
  REGION >4: note = The entire sequence of amino acids 1-4 can be repeated one or more times.
GGSG

SEQ ID NO: 124 - protein, 5 aa, synthetic construct
  REGION >5: note = The entire sequence of amino acids 1-5 can be repeated one or more times.
GGSGG

SEQ ID NO: 125 - protein, 5 aa, synthetic construct
  REGION >5: note = The entire sequence of amino acids 1-5 can be repeated one or more times.
GGGGS

SEQ ID NO: 126 - protein, 20 aa, synthetic construct
GSGSGSGSGSGSGSGSGSGS

SEQ ID NO: 127 - protein, 30 aa, synthetic construct
GGSGGSGGSGGSGGSGGSGGSGGSGGSGGS

SEQ ID NO: 128 - protein, 40 aa, synthetic construct
GGGSGGGSGGGSGGGSGGGSGGGSGGGSGGGSGGGSGGGS

SEQ ID NO: 129 - protein, 40 aa, synthetic construct
GGSGGGSGGGSGGGSGGGSGGGSGGGSGGGSGGGSGGGSG

SEQ ID NO: 130 - protein, 50 aa, synthetic construct
GGSGGGGSGGGGSGGGGSGGGGSGGGGSGGGGSGGGGSGGGGSGGGGSGG

SEQ ID NO: 131 - protein, 50 aa, synthetic construct
GGGGSGGGGSGGGGSGGGGSGGGGSGGGGSGGGGSGGGGSGGGGSGGGGS

SEQ ID NO: 132 - protein, 15 aa, synthetic construct
GGGGSGGGGSGGGGS

SEQ ID NO: 133 - protein, 100 aa, synthetic construct
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 134 - protein, 100 aa, synthetic construct
GGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGG

SEQ ID NO: 135 - protein, 119 aa, synthetic construct [sequence omitted]

SEQ ID NO: 136 - protein, 12 aa, synthetic construct
GGSVSSGDYYWT

SEQ ID NO: 137 - protein, 16 aa, synthetic construct
HIYYSGNTNYNPSLKS

SEQ ID NO: 138 - protein, 9 aa, synthetic construct
DRVTGAFDI

SEQ ID NO: 139 - protein, 107 aa, synthetic construct [sequence omitted]

SEQ ID NO: 140 - protein, 11 aa, synthetic construct
QASQDISNYLN

SEQ ID NO: 141 - protein, 11 aa, synthetic construct
LLIYDASNLET

SEQ ID NO: 142 - protein, 9 aa, synthetic construct
QHFDHLPLA

SEQ ID NO: 143 - protein, 246 aa, synthetic construct
DIQMTQSPSSLSASVGDRVTITCQASQDISNYLNWYQQKPGKAPKLLIYDASNLETGVPSRFSGSGSGTDFTFTISSLQPEDIATYFCQHFDHLPLAFGCGTKVEIKGGGGSGGGGSGGGGSGGGGSQVQLQESGPGLVKPSETLSLTCTVSGGSVSSGDYYWTWIRQSPGKCLEWIGHIYYSGNTNYNPSLKSRLTISIDTSKTQFSLKLSSVTAADTAIYYCVRDRVTGAFDIWGQGTMVTVSS

SEQ ID NO: 144 - protein, 246 aa, synthetic construct
QVQLQESGPGLVKPSETLSLTCTVSGGSVSSGDYYWTWIRQSPGKCLEWIGHIYYSGNTNYNPSLKSRLTISIDTSKTQFSLKLSSVTAADTAIYYCVRDRVTGAFDIWGQGTMVTVSSGGGGSGGGGSGGGGSGGGGSDIQMTQSPSSLSASVGDRVTITCQASQDISNYLNWYQQKPGKAPKLLIYDASNLETGVPSRFSGSGSGTDFTFTISSLQPEDIATYFCQHFDHLPLAFGCGTKVEIK

SEQ ID NO: 145 - protein, 119 aa, synthetic construct
QVQLQESGPGLVKPSETLSLTCTVSGGSVSSGDYYWTWIRQSPGKGLEWIGHIYYSGNTNYNPRLKSRLTISIDTSKTQFSLKLSSVTAADTAIYYCVRDRVTGAFDIWGQGTMVTVSS

SEQ ID NO: 146 - protein, 16 aa, synthetic construct
HIYYSGNTNYNPRLKS

SEQ ID NO: 147 - protein, 107 aa, synthetic construct
DIQMTQSPSSLSASVGDRVTITCQASQDISNYLNWYQQKPGKAPKLLIYDASNLETGVPSRFSGSGSGTDFTFTISSLQPEDIATYYCQHFDHLPLAFGGGTKVEIK

SEQ ID NO: 148 - protein, 246 aa, synthetic construct
DIQMTQSPSSLSASVGDRVTITCQASQDISNYLNWYQQKPGKAPKLLIYDASNLETGVPSRFSGSGSGTDFTFTISSLQPEDIATYYCQHFDHLPLAFGCGTKVEIKGGGGSGGGGSGGGGSGGGGSQVQLQESGPGLVKPSETLSLTCTVSGGSVSSGDYYWTWIRQSPGKCLEWIGHIYYSGNTNYNPRLKSRLTISIDTSKTQFSLKLSSVTAADTAIYYCVRDRVTGAFDIWGQGTMVTVSS

SEQ ID NO: 149 - protein, 246 aa, synthetic construct
QVQLQESGPGLVKPSETLSLTCTVSGGSVSSGDYYWTWIRQSPGKCLEWIGHIYYSGNTNYNPRLKSRLTISIDTSKTQFSLKLSSVTAADTAIYYCVRDRVTGAFDIWGQGTMVTVSSGGGGSGGGGSGGGGSGGGGSDIQMTQSPSSLSASVGDRVTITCQASQDISNYLNWYQQKPGKAPKLLIYDASNLETGVPSRFSGSGSGTDFTFTISSLQPEDIATYYCQHFDHLPLAFGCGTKVEIK

SEQ ID NO: 150 - protein, 107 aa, synthetic construct
DIQMTQSPSSLSASVGDRVTITCQASQDISNYLNWYQQKPGKAPKLLIYDASNLETGVPSRFSGSGSGTDFTFTISSLQPEDIATYYCQHFRHLPLAFGGGTKVEIK

SEQ ID NO: 151 - protein, 9 aa, synthetic construct
QHFRHLPLA

SEQ ID NO: 152 - protein, 246 aa, synthetic construct
DIQMTQSPSSLSASVGDRVTITCQASQDISNYLNWYQQKPGKAPKLLIYDASNLETGVPSRFSGSGSGTDFTFTISSLQPEDIATYYCQHFRHLPLAFGCGTKVEIKGGGGSGGGGSGGGGSGGGGSQVQLQESGPGLVKPSETLSLTCTVSGGSVSSGDYYWTWIRQSPGKCLEWIGHIYYSGNTNYNPRLKSRLTISIDTSKTQFSLKLSSVTAADTAIYYCVRDRVTGAFDIWGQGTMVTVSS

SEQ ID NO: 153 - protein, 246 aa, synthetic construct
QVQLQESGPGLVKPSETLSLTCTVSGGSVSSGDYYWTWIRQSPGKCLEWIGHIYYSGNTNYNPRLKSRLTISIDTSKTQFSLKLSSVTAADTAIYYCVRDRVTGAFDIWGQGTMVTVSSGGGGSGGGGSGGGGSGGGGSDIQMTQSPSSLSASVGDRVTITCQASQDISNYLNWYQQKPGKAPKLLIYDASNLETGVPSRFSGSGSGTDFTFTISSLQPEDIATYYCQHFRHLPLAFGCGTKVEIK

SEQ ID NO: 154 - protein, 246 aa, synthetic construct
DIQMTQSPSSLSASVGDRVTITCQASQDISNYLNWYQQKPGKAPKLLIYDASNLETGVPSRFSGSGSGTDFTFTISSLQPEDIATYYCQHFRHLPLAFGCGTKVEIKGGGGSGGGGSGGGGSGGGGSQVQLQESGPGLVKPSETLSLTCTVSGGSVSSGDYYWTWIRQSPGKCLEWIGHIYYSGNTNYNPSLKSRLTISIDTSKTQFSLKLSSVTAADTAIYYCVRDRVTGAFDIWGQGTMVTVSS

SEQ ID NO: 155 - protein, 246 aa, synthetic construct
QVQLQESGPGLVKPSETLSLTCTVSGGSVSSGDYYWTWIRQSPGKCLEWIGHIYYSGNTNYNPSLKSRLTISIDTSKTQFSLKLSSVTAADTAIYYCVRDRVTGAFDIWGQGTMVTVSSGGGGSGGGGSGGGGSGGGGSDIQMTQSPSSLSASVGDRVTITCQASQDISNYLNWYQQKPGKAPKLLIYDASNLETGVPSRFSGSGSGTDFTFTISSLQPEDIATYYCQHFRHLPLAFGCGTKVEIK

SEQ ID NO: 156 - protein, 119 aa, synthetic construct
  VARIANT 64: note = X can be R or S
QVQLQESGPGLVKPSETLSLTCTVSGGSVSSGDYYWTWIRQSPGKGLEWIGHIYYSGNTNYNPXLKSRLTISIDTSKTQFSLKLSSVTAADTAIYYCVRDRVTGAFDIWGQGTMVTVSS

SEQ ID NO: 157 - protein, 16 aa, synthetic construct
  VARIANT 13: note = X can be R or S
HIYYSGNTNYNPXLKS

SEQ ID NO: 158 - protein, 246 aa, synthetic construct
  VARIANT 191: note = X can be R or S
DIQMTQSPSSLSASVGDRVTITCQASQDISNYLNWYQQKPGKAPKLLIYDASNLETGVPSRFSGSGSGTDFTFTISSLQPEDIATYYCQHFRHLPLAFGCGTKVEIKGGGGSGGGGSGGGGSGGGGSQVQLQESGPGLVKPSETLSLTCTVSGGSVSSGDYYWTWIRQSPGKCLEWIGHIYYSGNTNYNPXLKSRLTISIDTSKTQFSLKLSSVTAADTAIYYCVRDRVTGAFDIWGQGTMVTVSS

SEQ ID NO: 159 - protein, 246 aa, synthetic construct
  VARIANT 64: note = X can be R or S
QVQLQESGPGLVKPSETLSLTCTVSGGSVSSGDYYWTWIRQSPGKCLEWIGHIYYSGNTNYNPXLKSRLTISIDTSKTQFSLKLSSVTAADTAIYYCVRDRVTGAFDIWGQGTMVTVSSGGGGSGGGGSGGGGSGGGGSDIQMTQSPSSLSASVGDRVTITCQASQDISNYLNWYQQKPGKAPKLLIYDASNLETGVPSRFSGSGSGTDFTFTISSLQPEDIATYYCQHFRHLPLAFGCGTKVEIK

SEQ ID NO: 160 - protein, 1210 aa, synthetic construct [sequence omitted]

SEQ ID NO: 161 - protein, 405 aa, synthetic construct
MRPSGTAGAALLALLAALCPASRALEEKKVCQGTSNKLTQLGTFEDHFLSLQRMFNNCEVVLGNLEITYVQRNYDLSFLKTIQEVAGYVLIALNTVERIPLENLQIIRGNMYYENSYALAVLSNYDANKTGLKELPMRNLQEILHGAVRFSNNPALCNVESIQWRDIVSSDFLSNMSMDFQNHLGSCQKCDPSCPNGSCWGAGEENCQKLTKIICAQQCSGRCRGKSPSDCCHNQCAAGCTGPRESDCLVCRKFRDEATCKDTCPPLMLYNPTTYQMDVNPEGKYSFGATCVKKCPRNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGLS

SEQ ID NO: 162 - protein, 705 aa, synthetic construct
MRPSGTAGAALLALLAALCPASRALEEKKVCQGTSNKLTQLGTFEDHFLSLQRMFNNCEVVLGNLEITYVQRNYDLSFLKTIQEVAGYVLIALNTVERIPLENLQIIRGNMYYENSYALAVLSNYDANKTGLKELPMRNLQEILHGAVRFSNNPALCNVESIQWRDIVSSDFLSNMSMDFQNHLGSCQKCDPSCPNGSCWGAGEENCQKLTKIICAQQCSGRCRGKSPSDCCHNQCAAGCTGPRESDCLVCRKFRDEATCKDTCPPLMLYNPTTYQMDVNPEGKYSFGATCVKKCPRNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGPGNESLKAMLFCLFKLSSCNQSNDGSVSHQSGSPAAQESCLGWIPSLLPSEFQLGWGGCSHLHAWPSASVIITASSCH

SEQ ID NO: 163 - protein, 628 aa, synthetic construct
MRPSGTAGAALLALLAALCPASRALEEKKVCQGTSNKLTQLGTFEDHFLSLQRMFNNCEVVLGNLEITYVQRNYDLSFLKTIQEVAGYVLIALNTVERIPLENLQIIRGNMYYENSYALAVLSNYDANKTGLKELPMRNLQEILHGAVRFSNNPALCNVESIQWRDIVSSDFLSNMSMDFQNHLGSCQKCDPSCPNGSCWGAGEENCQKLTKIICAQQCSGRCRGKSPSDCCHNQCAAGCTGPRESDCLVCRKFRDEATCKDTCPPLMLYNPTTYQMDVNPEGKYSFGATCVKKCPRNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGS

SEQ ID NO: 164 - protein, 451 aa, synthetic construct
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGAPIGAAAGWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTENQVSLTCLVKGFYPSDIAVEWESNGQPENNYKTTPPVLDSDGSFFLYSWLTVDKSRWQQGNVFSCSVMHEALHNHYTQKSLSLSPG

SEQ ID NO: 165 - protein, 214 aa, synthetic construct
DIQMTQSPSSVSASVGDRVTITCRASQGISSWLAWYQQKPGKAPKLLIYAASSLQSGVPSRFSGSGSGTDFTLTISSLQPEDFATYYCQQGVSFPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 166 - protein, 474 aa, synthetic construct
DIQMTQSPSSLSASVGDRVTITCQASQDISNYLNWYQQKPGKAPKLLIYDASNLETGVPSRFSGSGSGTDFTFTISSLQPEDIATYYCQHFDHLPLAFGCGTKVEIKGGGGSGGGGSGGGGSGGGGSQVQLQESGPGLVKPSETLSLTCTVSGGSVSSGDYYWTWIRQSPGKCLEWIGHIYYSGNTNYNPRLKSRLTISIDTSKTQFSLKLSSVTAADTAIYYCVRDRVTGAFDIWGQGTMVTVSSGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPCRDELTKNQVSLTCLVKGFYPSDIAVEWESNGQPENNYKTTPPVLVSDGSFTLYSKLTVDKSRWQQGNVFSCSVMHEALHNHYTQKSLSLSPG

SEQ ID NO: 167 - protein, 474 aa, synthetic construct
DIQMTQSPSSLSASVGDRVTITCQASQDISNYLNWYQQKPGKAPKLLIYDASNLETGVPSRFSGSGSGTDFTFTISSLQPEDIATYYCQHFRHLPLAFGCGTKVEIKGGGGSGGGGSGGGGSGGGGSQVQLQESGPGLVKPSETLSLTCTVSGGSVSSGDYYWTWIRQSPGKCLEWIGHIYYSGNTNYNPRLKSRLTISIDTSKTQFSLKLSSVTAADTAIYYCVRDRVTGAFDIWGQGTMVTVSSGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPCRDELTKNQVSLTCLVKGFYPSDIAVEWESNGQPENNYKTTPPVLVSDGSFTLYSKLTVDKSRWQQGNVFSCSVMHEALHNHYTQKSLSLSPG

SEQ ID NO: 168 - protein, 474 aa, synthetic construct
DIQMTQSPSSLSASVGDRVTITCQASQDISNYLNWYQQKPGKAPKLLIYDASNLETGVPSRFSGSGSGTDFTFTISSLQPEDIATYYCQHFRHLPLAFGCGTKVEIKGGGGSGGGGSGGGGSGGGGSQVQLQESGPGLVKPSETLSLTCTVSGGSVSSGDYYWTWIRQSPGKCLEWIGHIYYSGNTNYNPSLKSRLTISIDTSKTQFSLKLSSVTAADTAIYYCVRDRVTGAFDIWGQGTMVTVSSGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPCRDELTKNQVSLTCLVKGFYPSDIAVEWESNGQPENNYKTTPPVLVSDGSFTLYSKLTVDKSRWQQGNVFSCSVMHEALHNHYTQKSLSLSPG

SEQ ID NO: 169 - skipped

SEQ ID NO: 170 - protein, 119 aa, synthetic construct
QVQLQESGPGLVKPSETLSLTCTVSGGSVSSGDYYWTWIRQSPGKCLEWIGHIYYSGNTNYNPRLKSRLTISIDTSKTQFSLKLSSVTAADTAIYYCVRDRVTGAFDIWGQGTMVTVSS

SEQ ID NO: 171 - protein, 107 aa, synthetic construct
DIQMTQSPSSLSASVGDRVTITCQASQDISNYLNWYQQKPGKAPKLLIYDASNLETGVPSRFSGSGSGTDFTFTISSLQPEDIATYYCQHFRHLPLAFGCGTKVEIK

SEQ ID NO: 172 - protein, 474 aa, synthetic construct
DIQMTQSPSSLSASVGDRVTITCQASQDISNYLNWYQQKPGKAPKLLIYDASNLETGVPSRFSGSGSGTDFTFTISSLQPEDIATYFCQHFDHLPLAFGCGTKVEIKGGGGSGGGGSGGGGSGGGGSQVQLQESGPGLVKPSETLSLTCTVSGGSVSSGDYYWTWIRQSPGKCLEWIGHIYYSGNTNYNPSLKSRLTISIDTSKTQFSLKLSSVTAADTAIYYCVRDRVTGAFDIWGQGTMVTVSSGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPCRDELTKNQVSLTCLVKGFYPSDIAVEWESNGQPENNYKTTPPVLVSDGSFTLYSKLTVDKSRWQQGNVFSCSVMHEALHNHYTQKSLSLSPG

SEQ ID NO: 173 - protein, 7 aa, synthetic construct
GFTFSSY

SEQ ID NO: 174 - protein, 6 aa, synthetic construct
SSSSSY

(19) United States
(12) Patent Application Publication
(10) Pub. No.: US 2023/0250176 A1
(43) Pub. Date: Aug. 10, 2023

(54) PPHARMACEUTICAL FORMULATIONS AND THERAPEUTIC USES OF MULTI-SPECIFIC BINDING PROTEINS THAT BIND EGFR, NKG2D, AND CD16

(71) Applicant: Dragonfly Therapeutics, Inc., Waltham, MA (US)

(72) Inventors: Ann F. Cheung, Lincoln, MA (US); Jean-Marie Cuillerot, Somerville, MA (US); Mark DeRose, Wilmington, MA (US); Stacey V. Drabic, Cambridge, MA (US); Asya Grinberg, Lexington, MA (US); Zong Sean Juo, Taipei (TW); Aaron Levin, Somerville, MA (US); Katia Liharska, Billerica, MA (US); Lynn Markowitz, Concord, MA (US); Christopher Ryan Morgan, Southborough, MA (US); Avni Shah, Framingham, MA (US); Michael Shifrin, Medford, MA (US); Nicolai Wagtmann, Concord, MA (US)

(21) Appl. No.: 18/166,769

(22) Filed: Feb. 9, 2023

Related U.S. Application Data
(60) Provisional application No. 63/349,621, filed on Jun. 7, 2022.
(60) Provisional application No. 63/308,420, filed on Feb. 9, 2022.

(51) Int. Cl. C07K 16/28; A61K 47/12; A61K 47/26; A61K 39/395; A61K 31/138; A61K 31/167; A61K 31/513; A61K 33/243; C07K 1/22; A61P 35/00

(52) U.S. Cl. CPC C07K 16/2863; C07K 16/2851; A61K 47/12; A61K 47/26; C07K 16/2818; C07K 16/2827; A61K 39/3955; A61K 31/138; A61K 31/167; A61K 31/513; A61K 33/243; C07K 1/22; A61P 35/00; C07K 2317/31; C07K 2317/622; C07K 2317/55

(57) ABSTRACT

Described herein are pharmaceutical formulations comprising multi-specific binding proteins that bind to NKG2D, CD16, and epidermal growth factor receptor (EGFR); and therapeutic uses of the multi-specific binding proteins and pharmaceutical formulations thereof for treating a disease, for example, cancer, in a patient in need thereof.

### CROSS-REFERENCE TO RELATED APPLICATIONS

This application claims priority to and the benefit of U.S. Provisional Patent Application No. 63/349,621, filed on Jun. 7, 2022, and U.S. Provisional Patent Application No. 63/308,420, filed on Feb. 9, 2022, the entire contents of each of which are incorporated by reference herein for all purposes.

### SEQUENCE LISTING

The instant application contains a Sequence Listing which has been submitted electronically in XML format and is hereby incorporated by reference in its entirety. Said XML file copy, created on Feb. 9, 2023, is named DFY-125US_SL.xml and is 171,292 bytes in size.

### FIELD OF THE INVENTION

The present application relates to pharmaceutical formulations including multi-specific binding proteins that bind to NKG2D, CD16, and epidermal growth factor receptor (EGFR); and therapeutic uses of the multi-specific binding proteins and pharmaceutical formulations thereof for treating a disease, for example, cancer, in a patient in need thereof.

### BACKGROUND

Cancer immunotherapies are desirable because they are highly specific and can facilitate destruction of cancer cells using the patient's own immune system. Fusion proteins such as bi-specific T-cell engagers are cancer immunotherapies described in the literature that bind to tumor cells and T-cells to facilitate destruction of tumor cells. Antibodies that bind to certain tumor-associated antigens have been described in the literature. See, e.g., WO 2016/134371 and WO 2015/095412.

Natural killer (NK) cells are a component of the innate immune system and make up approximately 15% of circulating lymphocytes. NK cells respond to signals through a variety of activating and inhibitory receptors on their surface. For example, when NK cells encounter healthy self-cells, their activity is inhibited through activation of the killer-cell immunoglobulin-like receptors (KIRs). Alternatively, when NK cells encounter foreign cells or cancer cells, they are activated via their activating receptors (e.g., NKG2D, NCRs, DNAM1). NK cells are also activated by the constant region of some immunoglobulins through CD16 receptors on their surface. The overall sensitivity of NK cells to activation depends on the sum of stimulatory and inhibitory signals. NKG2D is a type-II transmembrane protein that is expressed by essentially all natural killer cells where NKG2D serves as an activating receptor. NKG2D is also found on T cells where it acts as a costimulatory receptor. The ability to modulate NK cell function via NKG2D is useful in various therapeutic contexts including malignancy.

The epidermal growth factor receptor (EGFR; ErbB-1; HER1) is a transmembrane protein that is a receptor for members of the epidermal growth factor family (EGF family) of extracellular protein ligands. Upon binding of its specific ligands, including epidermal growth factor and transforming growth factor α (TGFα), EGFR undergoes a transition from an inactive monomeric form to an active homodimer or heterodimer with other ErbB family receptors. The dimerization stimulates its intrinsic intracellular protein-tyrosine kinase activity, and elicits downstream signaling cascades, leading to DNA synthesis and cell proliferation. EGFR is involved in modulation of phenotypes such as cell migration, adhesion, and proliferation.

Mutations that lead to epidermal growth factor receptor (EGFR) overexpression or overactivity have been associated with a number of cancers, including non-small cell lung cancer, anal cancers, glioblastoma and epithelial tumors of the head and neck. These somatic mutations involving EGFR lead to its constant activation, which produces uncontrolled cell division. In glioblastoma a more or less specific mutation of EGFR, called EGFRvIII is often observed. Mutations, amplifications or misregulations of EGFR or family members are implicated in other solid tumors, including colorectal cancer, renal cell carcinoma, bladder cancer, cervical cancer, ovarian cancer, pancreatic cancer, and liver cancer.

Anti-EGFR monoclonal antibodies, such as cetuximab, panitumumab, necitumumab, and zalutumumab, have been developed. Multi-specific binding proteins that bind EGFR and one or more immune cell surface proteins have been studied. For example, WO 2019/035939 describes multi-specific binding proteins that bind EGFR, NKG2D, and CD16. The present disclosure adds to these developments and provides clinical methods, including dosage regimens, to treat patients with specific EGFR-targeting cancer immunotherapies with desired safety and efficacy. Furthermore, the present disclosure adds to the earlier developments in the field by providing formulations including such cancer immunotherapies that are sufficiently stable and suitable for administration to patients.

### SUMMARY

Disclosed herein, in various embodiments is a method of treating unresectable solid tumor in a subject in need thereof, including administering an effective amount of a multi-specific binding protein including: (a) a first antigen-binding site that binds NKG2D; (b) a second antigen-binding site that binds EGFR; and (c) an antibody Fc domain or a portion thereof sufficient to bind CD16, or a third antigen-binding site that binds CD16. In some embodiments, the second antigen-binding site that binds EGFR has: (i) a VH having CDR1, CDR2, and CDR3 sequences of SEQ ID NOs: 136, 157, and 138, respectively; and a VL having CDR1, CDR2, and CDR3 sequences of SEQ ID NOs: 140, 141, and 151, respectively; (ii) a VH having CDR1, CDR2, and CDR3 sequences of SEQ ID NOs: 136, 146, and 138, respectively; and a VL having CDR1, CDR2, and CDR3 sequences of SEQ ID NOs: 140, 141, and 142, respectively; (iii) a VH having CDR1, CDR2, and CDR3 sequences of SEQ ID NOs: 136, 146, and 138, respectively; and a VL having CDR1, CDR2, and CDR3 sequences of SEQ ID NOs: 140, 141, and 151, respectively; (iv) a VH having CDR1, CDR2, and CDR3 sequences of SEQ ID NOs: 136, 137, and 138, respectively; and a VL having CDR1, CDR2, and CDR3 sequences of SEQ ID NOs: 140, 141, and 151, respectively; or (v) a VH having CDR1, CDR2, and CDR3 sequences of SEQ ID NOs: 136, 137, and 138, respectively; and a VL having CDR1, CDR2, and CDR3 sequences of SEQ ID NOs: 140, 141, and 142, respectively.

Also disclosed herein, in various embodiments, is a method of treating a recurrent solid tumor in a subject in need thereof, including administering an effective amount of a multi-specific binding protein including: (a) a first antigen-binding site that binds NKG2D; (b) a second antigen-binding site that binds EGFR; and (c) an antibody Fc domain or a portion thereof sufficient to bind CD16, or a third antigen-binding site that binds CD16. In some embodiments, the second antigen-binding site that binds EGFR has: (i) a VH having CDR1, CDR2, and CDR3 sequences of SEQ ID NOs: 136, 157, and 138, respectively; and a VL having CDR1, CDR2, and CDR3 sequences of SEQ ID NOs: 140, 141, and 151, respectively; (ii) a VH having CDR1, CDR2, and CDR3 sequences of SEQ ID NOs: 136, 146, and 138, respectively; and a VL having CDR1, CDR2, and CDR3 sequences of SEQ ID NOs: 140, 141, and 142, respectively; (iii) a VH having CDR1, CDR2, and CDR3 sequences of SEQ ID NOs: 136, 146, and 138, respectively; and a VL having CDR1, CDR2, and CDR3 sequences of SEQ ID NOs: 140, 141, and 151, respectively; (iv) a VH having CDR1, CDR2, and CDR3 sequences of SEQ ID NOs: 136, 137, and 138, respectively; and a VL having CDR1, CDR2, and CDR3 sequences of SEQ ID NOs: 140, 141, and 151, respectively; or (v) a VH having CDR1, CDR2, and CDR3 sequences of SEQ ID NOs: 136, 137, and 138, respectively; and a VL having CDR1, CDR2, and CDR3 sequences of SEQ ID NOs: 140, 141, and 142, respectively.

Also disclosed herein, in various embodiments, is a method of treating advanced solid tumors for which there is no effective standard therapy in a subject in need thereof, including administering an effective amount of a multi-specific binding protein including: (a) a first antigen-binding site that binds NKG2D; (b) a second antigen-binding site that binds EGFR; and (c) an antibody Fc domain or a portion thereof sufficient to bind CD16, or a third antigen-binding site that binds CD16. In some embodiments, the second antigen-binding site that binds EGFR has: (i) a VH having CDR1, CDR2, and CDR3 sequences of SEQ ID NOs: 136, 157, and 138, respectively; and a VL having CDR1, CDR2, and CDR3 sequences of SEQ ID NOs: 140, 141, and 151, respectively; (ii) a VH having CDR1, CDR2, and CDR3 sequences of SEQ ID NOs: 136, 146, and 138, respectively; and a VL having CDR1, CDR2, and CDR3 sequences of SEQ ID NOs: 140, 141, and 142, respectively; (iii) a VH having CDR1, CDR2, and CDR3 sequences of SEQ ID NOs: 136, 146, and 138, respectively; and a VL having CDR1, CDR2, and CDR3 sequences of SEQ ID NOs: 140, 141, and 151, respectively; (iv) a VH having CDR1, CDR2, and CDR3 sequences of SEQ ID NOs: 136, 137, and 138, respectively; and a VL having CDR1, CDR2, and CDR3 sequences of SEQ ID NOs: 140, 141, and 151, respectively; or (v) a VH having CDR1, CDR2, and CDR3 sequences of SEQ ID NOs: 136, 137, and 138, respectively; and a VL having CDR1, CDR2, and CDR3 sequences of SEQ ID NOs: 140, 141, and 142, respectively.

Also disclosed herein, in various embodiments, is a method of treating cancer in a subject who is intolerant of standard cancer therapies, including administering an effective amount of a multi-specific binding protein including: (a) a first antigen-binding site that binds NKG2D; (b) a second antigen-binding site that binds EGFR; and (c) an antibody Fc domain or a portion thereof sufficient to bind CD16, or a third antigen-binding site that binds CD16. In some embodiments, the second antigen-binding site that binds EGFR has: (i) a VH having CDR1, CDR2, and CDR3 sequences of SEQ ID NOs: 136, 157, and 138, respectively; and a VL having CDR1, CDR2, and CDR3 sequences of SEQ ID NOs: 140, 141, and 151, respectively; (ii) a VH having CDR1, CDR2, and CDR3 sequences of SEQ ID NOs: 136, 146, and 138, respectively; and a VL having CDR1, CDR2, and CDR3 sequences of SEQ ID NOs: 140, 141, and 142, respectively; (iii) a VH having CDR1, CDR2, and CDR3 sequences of SEQ ID NOs: 136, 146, and 138, respectively; and a VL having CDR1, CDR2, and CDR3 sequences of SEQ ID NOs: 140, 141, and 151, respectively; (iv) a VH having CDR1, CDR2, and CDR3 sequences of SEQ ID NOs: 136, 137, and 138, respectively; and a VL having CDR1, CDR2, and CDR3 sequences of SEQ ID NOs: 140, 141, and 151, respectively; or (v) a VH having CDR1, CDR2, and CDR3 sequences of SEQ ID NOs: 136, 137, and 138, respectively; and a VL having CDR1, CDR2, and CDR3 sequences of SEQ ID NOs: 140, 141, and 142, respectively.

Also disclosed herein, in various embodiments, is a method of treating cancer in a subject in need thereof, including administering an effective amount of a multi-specific binding protein in combination with nivolumab. The multi-specific binding protein includes: (a) a first antigen-binding site that binds NKG2D; (b) a second antigen-binding site that binds EGFR; and (c) an antibody Fc domain or a portion thereof sufficient to bind CD16, or a third antigen-binding site that binds CD16. In some embodiments, the second antigen-binding site that binds EGFR has: (i) a VH having CDR1, CDR2, and CDR3 sequences of SEQ ID NOs: 136, 157, and 138, respectively; and a VL having CDR1, CDR2, and CDR3 sequences of SEQ ID NOs: 140, 141, and 151, respectively; (ii) a VH having CDR1, CDR2, and CDR3 sequences of SEQ ID NOs: 136, 146, and 138, respectively; and a VL having CDR1, CDR2, and CDR3 sequences of SEQ ID NOs: 140, 141, and 142, respectively; (iii) a VH having CDR1, CDR2, and CDR3 sequences of SEQ ID NOs: 136, 146, and 138, respectively; and a VL having CDR1, CDR2, and CDR3 sequences of SEQ ID NOs: 140, 141, and 151, respectively; (iv) a VH having CDR1, CDR2, and CDR3 sequences of SEQ ID NOs: 136, 137, and 138, respectively; and a VL having CDR1, CDR2, and CDR3 sequences of SEQ ID NOs: 140, 141, and 151, respectively; or (v) a VH having CDR1, CDR2, and CDR3 sequences of SEQ ID NOs: 136, 137, and 138, respectively; and a VL having CDR1, CDR2, and CDR3 sequences of SEQ ID NOs: 140, 141, and 142, respectively.

Also disclosed herein, in various embodiments, is a method of treating cancer in a subject in need thereof, including administering 5 mg/kg to 50 mg/kg of a multi-specific binding protein including: (a) a first antigen-binding site that binds NKG2D; (b) a second antigen-binding site that binds EGFR; and (c) an antibody Fc domain or a portion thereof sufficient to bind CD16, or a third antigen-binding site that binds CD16. In some embodiments, the second antigen-binding site that binds EGFR has: (i) a VH having CDR1, CDR2, and CDR3 sequences of SEQ ID NOs: 136, 157, and 138, respectively; and a VL having CDR1, CDR2, and CDR3 sequences of SEQ ID NOs: 140, 141, and 151, respectively; (ii) a VH having CDR1, CDR2, and CDR3 sequences of SEQ ID NOs: 136, 146, and 138, respectively; and a VL having CDR1, CDR2, and CDR3 sequences of SEQ ID NOs: 140, 141, and 142, respectively; (iii) a VH having CDR1, CDR2, and CDR3 sequences of SEQ ID NOs: 136, 146, and 138, respectively; and a VL having CDR1, CDR2, and CDR3 sequences of SEQ ID NOs: 140, 141, and 151, respectively; (iv) a VH having CDR1, CDR2, and CDR3 sequences of SEQ ID NOs: 136, 137, and 138, respectively; and a VL having CDR1, CDR2, and CDR3 sequences of SEQ ID NOs: 140, 141, and 151, respectively; or (v) a VH having CDR1, CDR2, and CDR3 sequences of SEQ ID NOs: 136, 137, and 138, respectively; and a VL having CDR1, CDR2, and CDR3 sequences of SEQ ID NOs: 140, 141, and 142, respectively.

Also disclosed herein, in various embodiments, is a method of treating cancer in a subject in need thereof, including administering once weekly, in 4-week treatment cycles, a multi-specific binding protein that includes: (a) a first antigen-binding site that binds NKG2D; (b) a second antigen-binding site that binds EGFR; and (c) an antibody Fc domain or a portion thereof sufficient to bind CD16, or a third antigen-binding site that binds CD16. In some embodiments, the second antigen-binding site that binds EGFR has: (i) a VH having CDR1, CDR2, and CDR3 sequences of SEQ ID NOs: 136, 157, and 138, respectively; and a VL having CDR1, CDR2, and CDR3 sequences of SEQ ID NOs: 140, 141, and 151, respectively; (ii) a VH having CDR1, CDR2, and CDR3 sequences of SEQ ID NOs: 136, 146, and 138, respectively; and a VL having CDR1, CDR2, and CDR3 sequences of SEQ ID NOs: 140, 141, and 142, respectively; (iii) a VH having CDR1, CDR2, and CDR3 sequences of SEQ ID NOs: 136, 146, and 138, respectively; and a VL having CDR1, CDR2, and CDR3 sequences of SEQ ID NOs: 140, 141, and 151, respectively; (iv) a VH having CDR1, CDR2, and CDR3 sequences of SEQ ID NOs: 136, 137, and 138, respectively; and a VL having CDR1, CDR2, and CDR3 sequences of SEQ ID NOs: 140, 141, and 151, respectively; or (v) a VH having CDR1, CDR2, and CDR3 sequences of SEQ ID NOs: 136, 137, and 138, respectively; and a VL having CDR1, CDR2, and CDR3 sequences of SEQ ID NOs: 140, 141, and 142, respectively.

Also disclosed herein, in various embodiments, is a method of treating cancer in a subject in need thereof and eligible for anti-PD-1 or an anti-PD-L1 therapy for a malignancy of epithelial origin. The subject is administered an effective amount of a multi-specific binding protein in combination with an anti-PD-1 or an anti-PD-L1 therapy; the multi-specific binding protein includes: (a) a first antigen-binding site that binds NKG2D, (b) a second antigen-binding site that binds EGFR, and (c) an antibody Fc domain or a portion thereof sufficient to bind CD16, or a third antigen-binding site that binds CD16. In some embodiments, the second antigen-binding site that binds EGFR has: (i) a VH having CDR1, CDR2, and CDR3 sequences of SEQ ID NOs: 136, 157, and 138, respectively; and a VL having CDR1, CDR2, and CDR3 sequences of SEQ ID NOs: 140, 141, and 151, respectively; (ii) a VH having CDR1, CDR2, and CDR3 sequences of SEQ ID NOs: 136, 146, and 138, respectively; and a VL having CDR1, CDR2, and CDR3 sequences of SEQ ID NOs: 140, 141, and 142, respectively; (iii) a VH having CDR1, CDR2, and CDR3 sequences of SEQ ID NOs: 136, 146, and 138, respectively; and a VL having CDR1, CDR2, and CDR3 sequences of SEQ ID NOs: 140, 141, and 151, respectively; (iv) a VH having CDR1, CDR2, and CDR3 sequences of SEQ ID NOs: 136, 137, and 138, respectively; and a VL having CDR1, CDR2, and CDR3 sequences of SEQ ID NOs: 140, 141, and 151, respectively; or (v) a VH having CDR1, CDR2, and CDR3 sequences of SEQ ID NOs: 136, 137, and 138, respectively; and a VL having CDR1, CDR2, and CDR3 sequences of SEQ ID NOs: 140, 141, and 142, respectively.

Also disclosed herein, in various embodiments, is a method of treating, in a subject in need thereof, a cancer for which no standard therapy exists, or a malignancy of epithelial origin for which standard therapy has failed. The method includes administering an effective amount of a multi-specific binding protein in combination with an anti-PD-1 or an anti-PD-L1 therapy; the multi-specific binding protein includes: (a) a first antigen-binding site that binds NKG2D, (b) a second antigen-binding site that binds EGFR, and (c) an antibody Fc domain or a portion thereof sufficient to bind CD16, or a third antigen-binding site that binds CD16. In some embodiments, the second antigen-binding site that binds EGFR has: (i) a VH having CDR1, CDR2, and CDR3 sequences of SEQ ID NOs: 136, 157, and 138, respectively; and a VL having CDR1, CDR2, and CDR3 sequences of SEQ ID NOs: 140, 141, and 151, respectively; (ii) a VH having CDR1, CDR2, and CDR3 sequences of SEQ ID NOs: 136, 146, and 138, respectively; and a VL having CDR1, CDR2, and CDR3 sequences of SEQ ID NOs: 140, 141, and 142, respectively; (iii) a VH having CDR1, CDR2, and CDR3 sequences of SEQ ID NOs: 136, 146, and 138, respectively; and a VL having CDR1, CDR2, and CDR3 sequences of SEQ ID NOs: 140, 141, and 151, respectively; (iv) a VH having CDR1, CDR2, and CDR3 sequences of SEQ ID NOs: 136, 137, and 138, respectively; and a VL having CDR1, CDR2, and CDR3 sequences of SEQ ID NOs: 140, 141, and 151, respectively; or (v) a VH having CDR1, CDR2, and CDR3 sequences of SEQ ID NOs: 136, 137, and 138, respectively; and a VL having CDR1, CDR2, and CDR3 sequences of SEQ ID NOs: 140, 141, and 142, respectively.

Also disclosed herein, in various embodiments, is a method of treating cancer in a subject in need thereof who has previously received an anti-PD-1 or anti-PD-L1 therapy, including administering an effective amount of a multi-specific binding protein in combination with an anti-PD-1 or an anti-PD-L1 therapy; the multi-specific binding protein includes: (a) a first antigen-binding site that binds NKG2D, (b) a second antigen-binding site that binds EGFR, and (c) an antibody Fc domain or a portion thereof sufficient to bind CD16, or a third antigen-binding site that binds CD16. In some embodiments, the second antigen-binding site that binds EGFR has: (i) a VH having CDR1, CDR2, and CDR3 sequences of SEQ ID NOs: 136, 157, and 138, respectively; and a VL having CDR1, CDR2, and CDR3 sequences of SEQ ID NOs: 140, 141, and 151, respectively; (ii) a VH having CDR1, CDR2, and CDR3 sequences of SEQ ID NOs: 136, 146, and 138, respectively; and a VL having CDR1, CDR2, and CDR3 sequences of SEQ ID NOs: 140, 141, and 142, respectively; (iii) a VH having CDR1, CDR2, and CDR3 sequences of SEQ ID NOs: 136, 146, and 138, respectively; and a VL having CDR1, CDR2, and CDR3 sequences of SEQ ID NOs: 140, 141, and 151, respectively; (iv) a VH having CDR1, CDR2, and CDR3 sequences of SEQ ID NOs: 136, 137, and 138, respectively; and a VL having CDR1, CDR2, and CDR3 sequences of SEQ ID NOs: 140, 141, and 151, respectively; or (v) a VH having CDR1, CDR2, and CDR3 sequences of SEQ ID NOs: 136, 137, and 138, respectively; and a VL having CDR1, CDR2, and CDR3 sequences of SEQ ID NOs: 140, 141, and 142, respectively.

Also disclosed herein, in various embodiments, is a method of treating head and neck squamous cell carcinoma (HNSCC) in a subject in need thereof, including administering an effective amount of a multi-specific binding protein that includes: (a) a first antigen-binding site that binds NKG2D; (b) a second antigen-binding site that binds EGFR; and (c) an antibody Fc domain or a portion thereof sufficient to bind CD16, or a third antigen-binding site that binds CD16. In some embodiments, the HNSCC is relapsed, or metastatic, and/or the subject has had radiographic disease progression while on or after having received: (i) pembrolizumab and platinum/5FU; (ii) pembrolizumab monotherapy; or (iii) platinum/5FU and cetuximab. In some embodiments, the second antigen-binding site that binds EGFR has: (i) a VH having CDR1, CDR2, and CDR3 sequences of SEQ ID NOs: 136, 157, and 138, respectively; and a VL having CDR1, CDR2, and CDR3 sequences of SEQ ID NOs: 140, 141, and 151, respectively; (ii) a VH having CDR1, CDR2, and CDR3 sequences of SEQ ID NOs: 136, 146, and 138, respectively; and a VL having CDR1, CDR2, and CDR3 sequences of SEQ ID NOs: 140, 141, and 142, respectively; (iii) a VH having CDR1, CDR2, and CDR3 sequences of SEQ ID NOs: 136, 146, and 138, respectively; and a VL having CDR1, CDR2, and CDR3 sequences of SEQ ID NOs: 140, 141, and 151, respectively; (iv) a VH having CDR1, CDR2, and CDR3 sequences of SEQ ID NOs: 136, 137, and 138, respectively; and a VL having CDR1, CDR2, and CDR3 sequences of SEQ ID NOs: 140, 141, and 151, respectively; or (v) a VH having CDR1, CDR2, and CDR3 sequences of SEQ ID NOs: 136, 137, and 138, respectively; and a VL having CDR1, CDR2, and CDR3 sequences of SEQ ID NOs: 140, 141, and 142, respectively.

Also disclosed herein, in various embodiments, is a method of treating a relapsed and/or metastatic colorectal cancer (CRC) in a subject in need thereof, including administering an effective amount of a multi-specific binding protein including: (a) a first antigen-binding site that binds NKG2D; (b) a second antigen-binding site that binds EGFR; and (c) an antibody Fc domain or a portion thereof sufficient to bind CD16, or a third antigen-binding site that binds CD16.

Also disclosed herein, in various embodiments, is a method of treating colorectal cancer (CRC) in a subject in need thereof who (i) has not had prior treatment with an anti-PD-1 or an anti-PD-L1 therapy, (ii) does not have high mismatch repair/microsatellite instability, (iii) has radiographic disease progression while or after receiving treatment for advanced (recurrent/unresectable/metastatic) cancer, and/or (iv) who has been treated with FOLFOX, CAPOX, FOLFIRI, or FOLFOXIRI, with or without a biological agent. The method includes administering an effective amount of a multi-specific binding protein that includes: (a) a first antigen-binding site that binds NKG2D; (b) a second antigen-binding site that binds EGFR; and (c) an antibody Fc domain or a portion thereof sufficient to bind CD16, or a third antigen-binding site that binds CD16. In some embodiments, the multi-specific binding protein used for treating CRC includes a second antigen-binding site that binds EGFR, which has: (i) a VH having CDR1, CDR2, and CDR3 sequences of SEQ ID NOs: 136, 157, and 138, respectively; and a VL having CDR1, CDR2, and CDR3 sequences of SEQ ID NOs: 140, 141, and 151, respectively; (ii) a VH having CDR1, CDR2, and CDR3 sequences of SEQ ID NOs: 136, 146, and 138, respectively; and a VL having CDR1, CDR2, and CDR3 sequences of SEQ ID NOs: 140, 141, and 142, respectively; (iii) a VH having CDR1, CDR2, and CDR3 sequences of SEQ ID NOs: 136, 146, and 138, respectively; and a VL having CDR1, CDR2, and CDR3 sequences of SEQ ID NOs: 140, 141, and 151, respectively; (iv) a VH having CDR1, CDR2, and CDR3 sequences of SEQ ID NOs: 136, 137, and 138, respectively; and a VL having CDR1, CDR2, and CDR3 sequences of SEQ ID NOs: 140, 141, and 151, respectively; or (v) a VH having CDR1, CDR2, and CDR3 sequences of SEQ ID NOs: 136, 137, and 138, respectively; and a VL having CDR1, CDR2, and CDR3 sequences of SEQ ID NOs: 140, 141, and 142, respectively.

Also disclosed herein, in various embodiments, is a method of treating non-small-cell lung cancer (NSCLC) in a subject in need thereof who (i) has previously received an anti-PD-1 or anti-PD-L1 therapy and/or (ii) has recurrent or progressive disease during or after platinum doublet-based chemotherapy or within 6 months after completing platinum-based chemotherapy for local disease. The method includes administering an effective amount of a multi-specific binding protein that includes: (a) a first antigen-binding site that binds NKG2D; (b) a second antigen-binding site that binds EGFR; and (c) an antibody Fc domain or a portion thereof sufficient to bind CD16, or a third antigen-binding site that binds CD16. In some embodiments, the second antigen-binding site that binds EGFR has: (i) a VH having CDR1, CDR2, and CDR3 sequences of SEQ ID NOs: 136, 157, and 138, respectively; and a VL having CDR1, CDR2, and CDR3 sequences of SEQ ID NOs: 140, 141, and 151, respectively; (ii) a VH having CDR1, CDR2, and CDR3 sequences of SEQ ID NOs: 136, 146, and 138, respectively; and a VL having CDR1, CDR2, and CDR3 sequences of SEQ ID NOs: 140, 141, and 142, respectively; (iii) a VH having CDR1, CDR2, and CDR3 sequences of SEQ ID NOs: 136, 146, and 138, respectively; and a VL having CDR1, CDR2, and CDR3 sequences of SEQ ID NOs: 140, 141, and 151, respectively; (iv) a VH having CDR1, CDR2, and CDR3 sequences of SEQ ID NOs: 136, 137, and 138, respectively; and a VL having CDR1, CDR2, and CDR3 sequences of SEQ ID NOs: 140, 141, and 151, respectively; or (v) a VH having CDR1, CDR2, and CDR3 sequences of SEQ ID NOs: 136, 137, and 138, respectively; and a VL having CDR1, CDR2, and CDR3 sequences of SEQ ID NOs: 140, 141, and 142, respectively.

Also disclosed herein, in various embodiments is a method of treating esophageal adenocarcinoma in a subject in need thereof, including administering an effective amount of a multi-specific binding protein that includes: (a) a first antigen-binding site that binds NKG2D; (b) a second antigen-binding site that binds EGFR; and (c) an antibody Fc domain or a portion thereof sufficient to bind CD16, or a third antigen-binding site that binds CD16. In some embodiments, the second antigen-binding site that binds EGFR has: (i) a VH having CDR1, CDR2, and CDR3 sequences of SEQ ID NOs: 136, 157, and 138, respectively; and a VL having CDR1, CDR2, and CDR3 sequences of SEQ ID NOs: 140, 141, and 151, respectively; (ii) a VH having CDR1, CDR2, and CDR3 sequences of SEQ ID NOs: 136, 146, and 138, respectively; and a VL having CDR1, CDR2, and CDR3 sequences of SEQ ID NOs: 140, 141, and 142, respectively; (iii) a VH having CDR1, CDR2, and CDR3 sequences of SEQ ID NOs: 136, 146, and 138, respectively; and a VL having CDR1, CDR2, and CDR3 sequences of SEQ ID NOs: 140, 141, and 151, respectively; (iv) a VH having CDR1, CDR2, and CDR3 sequences of SEQ ID NOs: 136, 137, and 138, respectively; and a VL having CDR1, CDR2, and CDR3 sequences of SEQ ID NOs: 140, 141, and 151, respectively; or (v) a VH having CDR1, CDR2, and CDR3 sequences of SEQ ID NOs: 136, 137, and 138, respectively; and a VL having CDR1, CDR2, and CDR3 sequences of SEQ ID NOs: 140, 141, and 142, respectively.

Also disclosed herein, in various embodiments, is a method of treating triple-negative breast cancer in a subject in need thereof, including administering an effective amount of a multi-specific binding protein that includes: (a) a first antigen-binding site that binds NKG2D; (b) a second antigen-binding site that binds EGFR; and (c) an antibody Fc domain or a portion thereof sufficient to bind CD16, or a third antigen-binding site that binds CD16. In some embodiments, the second antigen-binding site that binds EGFR has: (i) a VH having CDR1, CDR2, and CDR3 sequences of SEQ ID NOs: 136, 157, and 138, respectively; and a VL having CDR1, CDR2, and CDR3 sequences of SEQ ID NOs: 140, 141, and 151, respectively; (ii) a VH having CDR1, CDR2, and CDR3 sequences of SEQ ID NOs: 136, 146, and 138, respectively; and a VL having CDR1, CDR2, and CDR3 sequences of SEQ ID NOs: 140, 141, and 142, respectively; (iii) a VH having CDR1, CDR2, and CDR3 sequences of SEQ ID NOs: 136, 146, and 138, respectively; and a VL having CDR1, CDR2, and CDR3 sequences of SEQ ID NOs: 140, 141, and 151, respectively; (iv) a VH having CDR1, CDR2, and CDR3 sequences of SEQ ID NOs: 136, 137, and 138, respectively; and a VL having CDR1, CDR2, and CDR3 sequences of SEQ ID NOs: 140, 141, and 151, respectively; or (v) a VH having CDR1, CDR2, and CDR3 sequences of SEQ ID NOs: 136, 137, and 138, respectively; and a VL having CDR1, CDR2, and CDR3 sequences of SEQ ID NOs: 140, 141, and 142, respectively.

Also disclosed herein, in various embodiments, is a method of treating renal cell carcinoma in a subject in need thereof, including administering an effective amount of a multi-specific binding protein or a formulation as disclosed herein, in various embodiments. The multi-specific binding protein includes: (a) a first antigen-binding site that binds NKG2D; (b) a second antigen-binding site that binds EGFR; and (c) an antibody Fc domain or a portion thereof sufficient to bind CD16, or a third antigen-binding site that binds CD16. In some embodiments, the second antigen-binding site that binds EGFR has: (i) a VH having CDR1, CDR2, and CDR3 sequences of SEQ ID NOs: 136, 157, and 138, respectively; and a VL having CDR1, CDR2, and CDR3 sequences of SEQ ID NOs: 140, 141, and 151, respectively; (ii) a VH having CDR1, CDR2, and CDR3 sequences of SEQ ID NOs: 136, 146, and 138, respectively; and a VL having CDR1, CDR2, and CDR3 sequences of SEQ ID NOs: 140, 141, and 142, respectively; (iii) a VH having CDR1, CDR2, and CDR3 sequences of SEQ ID NOs: 136, 146, and 138, respectively; and a VL having CDR1, CDR2, and CDR3 sequences of SEQ ID NOs: 140, 141, and 151, respectively; (iv) a VH having CDR1, CDR2, and CDR3 sequences of SEQ ID NOs: 136, 137, and 138, respectively; and a VL having CDR1, CDR2, and CDR3 sequences of SEQ ID NOs: 140, 141, and 151, respectively; or (v) a VH having CDR1, CDR2, and CDR3 sequences of SEQ ID NOs: 136, 137, and 138, respectively; and a VL having CDR1, CDR2, and CDR3 sequences of SEQ ID NOs: 140, 141, and 142, respectively.

Also disclosed herein, in various embodiments, is a method of treating gastric cancer in a subject in need thereof, including administering a formulation as disclosed herein, in various embodiments, including an effective amount of a multi-specific binding protein that includes: (a) a first antigen-binding site that binds NKG2D; (b) a second antigen-binding site that binds EGFR; and (c) an antibody Fc domain or a portion thereof sufficient to bind CD16, or a third antigen-binding site that binds CD16. In some embodiments, the second antigen-binding site that binds EGFR has: (i) a VH having CDR1, CDR2, and CDR3 sequences of SEQ ID NOs: 136, 157, and 138, respectively; and a VL having CDR1, CDR2, and CDR3 sequences of SEQ ID NOs: 140, 141, and 151, respectively; (ii) a VH having CDR1, CDR2, and CDR3 sequences of SEQ ID NOs: 136, 146, and 138, respectively; and a VL having CDR1, CDR2, and CDR3 sequences of SEQ ID NOs: 140, 141, and 142, respectively; (iii) a VH having CDR1, CDR2, and CDR3 sequences of SEQ ID NOs: 136, 146, and 138, respectively; and a VL having CDR1, CDR2, and CDR3 sequences of SEQ ID NOs: 140, 141, and 151, respectively; (iv) a VH having CDR1, CDR2, and CDR3 sequences of SEQ ID NOs: 136, 137, and 138, respectively; and a VL having CDR1, CDR2, and CDR3 sequences of SEQ ID NOs: 140, 141, and 151, respectively; or (v) a VH having CDR1, CDR2, and CDR3 sequences of SEQ ID NOs: 136, 137, and 138, respectively; and a VL having CDR1, CDR2, and CDR3 sequences of SEQ ID NOs: 140, 141, and 142, respectively.

Also disclosed herein, in various embodiments, is a method of treating pancreatic cancer in a subject in need thereof, including administering a formulation as disclosed herein, in various embodiments, including an effective amount of a multi-specific binding protein that includes: (a) a first antigen-binding site that binds NKG2D; (b) a second antigen-binding site that binds EGFR; and (c) an antibody Fc domain or a portion thereof sufficient to bind CD16, or a third antigen-binding site that binds CD16. In some embodiments, the second antigen-binding site that binds EGFR has: (i) a VH having CDR1, CDR2, and CDR3 sequences of SEQ ID NOs: 136, 157, and 138, respectively; and a VL having CDR1, CDR2, and CDR3 sequences of SEQ ID NOs: 140, 141, and 151, respectively; (ii) a VH having CDR1, CDR2, and CDR3 sequences of SEQ ID NOs: 136, 146, and 138, respectively; and a VL having CDR1, CDR2, and CDR3 sequences of SEQ ID NOs: 140, 141, and 142, respectively; (iii) a VH having CDR1, CDR2, and CDR3 sequences of SEQ ID NOs: 136, 146, and 138, respectively; and a VL having CDR1, CDR2, and CDR3 sequences of SEQ ID NOs: 140, 141, and 151, respectively; (iv) a VH having CDR1, CDR2, and CDR3 sequences of SEQ ID NOs: 136, 137, and 138, respectively; and a VL having CDR1, CDR2, and CDR3 sequences of SEQ ID NOs: 140, 141, and 151, respectively; or (v) a VH having CDR1, CDR2, and CDR3 sequences of SEQ ID NOs: 136, 137, and 138, respectively; and a VL having CDR1, CDR2, and CDR3 sequences of SEQ ID NOs: 140, 141, and 142, respectively.

Also disclosed herein, in various embodiments, is a method of treating cancer in a subject in need thereof, including administering: i) an effective amount of pre-medication including: (a) an antihistamine and an antipyretic; and/or (b) a corticosteroid, and ii) an effective amount of a multi-specific binding protein that includes: (i) a first antigen-binding site that binds NKG2D; (ii) a second antigen-binding site that binds EGFR, which has: 1) (i) a VH having CDR1, CDR2, and CDR3 sequences of SEQ ID NOs: 136, 157, and 138, respectively; and a VL having CDR1, CDR2, and CDR3 sequences of SEQ ID NOs: 140, 141, and 151, respectively; (ii) a VH having CDR1, CDR2, and CDR3 sequences of SEQ ID NOs: 136, 146, and 138, respectively; and a VL having CDR1, CDR2, and CDR3 sequences of SEQ ID NOs: 140, 141, and 142, respectively; (iii) a VH having CDR1, CDR2, and CDR3 sequences of SEQ ID NOs: 136, 146, and 138, respectively; and a VL having CDR1, CDR2, and CDR3 sequences of SEQ ID NOs: 140, 141, and 151, respectively; (iv) a VH having CDR1, CDR2, and CDR3 sequences of SEQ ID NOs: 136, 137, and 138, respectively; and a VL having CDR1, CDR2, and CDR3 sequences of SEQ ID NOs: 140, 141, and 151, respectively; or (v) a VH having CDR1, CDR2, and CDR3 sequences of SEQ ID NOs: 136, 137, and 138, respectively; and a VL having CDR1, CDR2, and CDR3 sequences of SEQ ID NOs: 140, 141, and 142, respectively; and (iii) an antibody Fc domain.

Also disclosed herein, in various embodiments, is a method of purifying a multi-specific protein including one or more steps selected from: a protein A affinity purification; a low pH viral inactivation; a mix-mode anion exchange chromatography; a mixed-mode chromatography; a viral filtration; and ultrafiltration/diafiltration. The multi-specific binding protein includes: (a) a first antigen-binding site that binds NKG2D; (b) a second antigen-binding site that binds EGFR; and (c) an antibody Fc domain or a portion thereof sufficient to bind CD16, or a third antigen-binding site that binds CD16.

Also disclosed herein, in various embodiments, is a pharmaceutical formulation including: (a) a multi-specific binding protein that includes: (i) a Fab that binds NKG2D; (ii) a single-chain variable fragment (scFv) that binds EGFR and includes: 1) a heavy chain variable domain (VH) having complementarity-determining region 1 (CDR1), complementarity-determining region 2 (CDR2), and complementarity-determining region 3 (CDR3) sequences of SEQ ID NOs: 136, 157, and 138, respectively; and a light chain variable domain (VL) having CDR1, CDR2, and CDR3 sequences of SEQ ID NOs: 140, 141, and 151, respectively; or 2) a VH having CDR1, CDR2, and CDR3 sequences of SEQ ID NOs: 136, 146, and 138, respectively; and a VL having CDR1, CDR2, and CDR3 sequences of SEQ ID NOs: 140, 141, and 142, respectively; and (iii) an antibody Fc domain; (b) citrate; (c) a sugar or sugar alcohol; and (d) a polysorbate, at pH 6.0 to 7.0.

Also disclosed herein, in various embodiments, is a pharmaceutical formulation including: (a) a multi-specific binding protein that includes: (i) a Fab that binds NKG2D; (ii) a single-chain variable fragment (scFv) that binds EGFR; and (iii) an antibody Fc domain, and (b) one or more of: (i) 15 mM to 25 mM citrate (e.g., 15 mM to 25 mM, 16 mM to 25 mM, 17 mM to 25 mM, 18 mM to 25 mM, 19 mM to 25 mM, 20 mM to 25 mM, 21 mM to 25 mM, 22 mM to 25 mM, 23 mM to 25 mM, 24 mM to 25 mM, 15 mM to 24 mM, 15 mM to 23 mM, 15 mM to 22 mM, 15 mM to 23 mM, 15 mM to 22 mM, 15 mM to 21 mM, 15 mM to 20 mM, 15 mM to 19 mM, 15 mM to 18 mM, 15 mM to 17 mM, 15 mM to 16 mM, or about 20 mM); and (ii) 4% to 8% (w/v) mannitol (e.g., 4% to 8% (w/v), 4% to 7% (w/v), 4% to 6% (w/v), 4% to 5% (w/v), 5% to 8% (w/v), 6% to 8% (w/v), 7% to 8% (w/v), or about 6% (w/v)), at pH 6.0 to 7.0.

Also disclosed herein, in various embodiments, is a method of inhibiting EGFR signaling in a subject in need thereof, including administering to the subject a multi-specific binding protein including: (i) a Fab that binds NKG2D; (ii) a single-chain variable fragment (scFv) that binds EGFR; and (iii) an antibody Fc domain.

These and other aspects and advantages of the methods and formulations described in the present application are illustrated by the following figures, detailed description and claims.

### BRIEF DESCRIPTION OF THE DRAWINGS

FIG. 1 is a representation of a heterodimeric, multi-specific binding antibody, e.g., a trispecific binding protein (TriNKET). Each arm can represent either the NKG2D-binding domain, or the binding domain corresponding to a tumor-associated antigen. In some embodiments, the NKG2D binding domain and the tumor-associated antigen binding domains can share a common light chain.

FIGS. 2A-2E illustrate five exemplary formats of a multi-specific binding protein, e.g., a trispecific binding protein (TriNKET). As shown in FIG. 2A, either the NKG2D-binding domain or the tumor-associated antigen binding domain can take the scFv format (left arm). An antibody that contains a NKG2D-targeting scFv, a tumor-associated antigen targeting Fab fragment, and a heterodimerized antibody constant region is referred herein as the F3-TriNKET. An antibody that contains a tumor-associated antigen targeting scFv, an NKG2D-targeting Fab fragment, and a heterodimerized antibody constant region/domain that binds CD16 is referred herein as the F3′-TriNKET (FIG. 2E). As shown in FIG. 2B, both the NKG2D-binding domain and tumor-associated antigen binding domain can take the scFv format. FIGS. 2C to 2D are illustrations of an antibody with three antigen-binding sites, including two antigen-binding sites that bind the tumor-associated antigen, and the NKG2D-binding site fused to the heterodimerized antibody constant region. These antibody formats are referred herein as F4-TriNKET. FIG. 2C illustrates that the two tumor-associated antigen-binding sites are in the Fab fragment format, and the NKG2D binding site in the scFv format. FIG. 2D illustrates that the tumor-associated antigen-binding sites are in the scFv format, and the NKG2D binding site is in the scFv format. FIG. 2E represents a trispecific antibody (TriNKET) that contains a tumor-targeting scFv, a NKG2D-targeting Fab fragment, and a heterodimerized antibody constant region/domain (“CD domain”) that binds CD16. The antibody format is referred herein as F3′-TriNKET. In certain exemplary multi-specific binding proteins, heterodimerization mutations on the antibody constant region include K360E and K409W on one constant domain; and Q347R, D399V and F405T on the opposite constant domain (shown as a triangular lock-and-key shape in the CD domains). The bold bar between the heavy and the light chain variable domains of the Fab fragments represents a disulfide bond.

FIG. 3 is a representation of a TriNKET in the Triomab form, which is a trifunctional, bispecific antibody that maintains an IgG-like shape. This chimera consists of two half antibodies, each with one light and one heavy chain, that originate from two parental antibodies. Triomab form may be a heterodimeric construct containing ½ of rat antibody and ½ of mouse antibody.

FIG. 4 is a representation of a TriNKET in the KiH Common Light Chain form, which involves the knobs-into-holes (KIHs) technology. KiH is a heterodimer containing 2 Fab fragments binding to target 1 and 2, and an Fc stabilized by heterodimerization mutations. TriNKET in the KiH format may be a heterodimeric construct with 2 Fab fragments binding to target 1 and target 2, containing two different heavy chains and a common light chain that pairs with both heavy chains.

FIG. 5 is a representation of a TriNKET in the dual-variable domain immunoglobulin (DVD-Ig™) form, which combines the target-binding domains of two monoclonal antibodies via flexible naturally occurring linkers, and yields a tetravalent IgG-like molecule. DVD-Ig™ is a homodimeric construct where variable domain targeting antigen 2 is fused to the N-terminus of a variable domain of a Fab fragment targeting antigen 1. DVD-Ig™ form contains normal Fc.

FIG. 6 is a representation of a TriNKET in the Orthogonal Fab fragment interface (Ortho-Fab) form, which is a heterodimeric construct that contains 2 Fab fragments binding to target 1 and target 2 fused to an Fc. Light chain (LC)-heavy chain (HC) pairing is ensured by orthogonal interface. Heterodimerization is ensured by mutations in the Fc.

FIG. 7 is a representation of a TriNKET in the 2-in-1 Ig format.

FIG. 8 is a representation of a TriNKET in the ES form, which is a heterodimeric construct containing two different Fab fragments binding to target 1 and target 2 fused to the Fc. Heterodimerization is ensured by electrostatic steering mutations in the Fc.

FIG. 9 is a representation of a TriNKET in the Fab Arm Exchange form: antibodies that exchange Fab fragment arms by swapping a heavy chain and attached light chain (half-molecule) with a heavy-light chain pair from another molecule, resulting in bispecific antibodies. Fab Arm Exchange form (cFae) is a heterodimer containing 2 Fab fragments binding to target 1 and 2, and an Fc stabilized by heterodimerization mutations.

FIG. 10 is a representation of a TriNKET in the SEED Body form, which is a heterodimer containing 2 Fab fragments binding to target 1 and 2, and an Fc stabilized by heterodimerization mutations.

FIG. 11 is a representation of a TriNKET in the LuZ-Y form, in which a leucine zipper is used to induce heterodimerization of two different HCs. The LuZ-Y form is a heterodimer containing two different scFabs binding to target 1 and 2, fused to an Fc. Heterodimerization is ensured through leucine zipper motifs fused to C-terminus of Fc.

FIG. 12 is a representation of a TriNKET in the Cov-X-Body form.

FIGS. 13A-13B are representations of TriNKETs in the κλ-Body forms, which are heterodimeric constructs with two different Fab fragments fused to an Fc stabilized by heterodimerization mutations: one Fab fragment targeting antigen 1 contains kappa LC, and the second Fab fragment targeting antigen 2 contains lambda LC. FIG. 13A is an exemplary representation of one form of a κλ-Body; FIG. 13B is an exemplary representation of another κλ-Body.

FIG. 14 is a representation of an Oasc-Fab heterodimeric construct that includes Fab fragment binding to target 1 and scFab binding to target 2, both of which are fused to the Fc domain. Heterodimerization is ensured by mutations in the Fc domain.

FIG. 15 is a representation of a DuetMab, which is a heterodimeric construct containing two different Fab fragments binding to antigens 1 and 2, and an Fc that is stabilized by heterodimerization mutations. Fab fragments 1 and 2 contain differential S-S bridges that ensure correct light chain and heavy chain pairing.

FIG. 16 is a representation of a CrossmAb, which is a heterodimeric construct with two different Fab fragments binding to targets 1 and 2, and an Fc stabilized by heterodimerization mutations. CL and CH1 domains, and VH and VL domains are switched, e.g., CH1 is fused in-line with VL, and CL is fused in-line with VH.

FIG. 17 is a representation of a Fit-Ig, which is a homodimeric construct where Fab fragment binding to antigen 2 is fused to the N-terminus of HC of Fab fragment that binds to antigen 1. The construct contains wild-type Fc.

FIGS. 18A-18B are chromatograms showing the prevalence of product-related species in a preparation of EGFR-TriNKET-3. FIG. 18A is an SEC-HPLC chromatogram. FIG. 18B is a CE-SDS (Non-Reducing) electropherogram.

FIGS. 19A-19C are graphs showing dose-responsive binding of EGFR-TriNKET, cetuximab, and panitumumab to epidermal growth factor receptor (EGFR)-expressing human tumor cell lines. FIG. 19A shows binding to Detroit 562 (pharyngeal carcinoma) cells. FIG. 19B shows binding to NCI-H1703 (non-small-cell lung cancer [NSCLC], squamous cell) cells. FIG. 19C shows binding to HT29 (colorectal adenocarcinoma) cells. Each point and error bars represent mean and standard deviation (SD), respectively, of fold-over-background of the median fluorescence intensity from duplicate wells.

FIGS. 20A-20B are graphs showing binding of EGFR-TriNKET, cetuximab, an hIgG1 isotype control, and a TriNKET isotype control with FcγR-silencing mutations (TriNKET isotype-FcγRsi) to immune cell subsets across 3 different healthy human donor samples. FIG. 20A shows binding to isolated peripheral blood mononuclear cells (PBMCs). FIG. 20A shows binding in whole blood. Dashes and error bars represent mean and SD, respectively, of molecules bound per cell across all 3 donors.

FIGS. 21A-21B are graphs showing dose-responsive binding of EGFR-TriNKET, a TriNKET isotype control, cetuximab, and panitumumab to the indicated cell populations. FIG. 21A shows binding to the parental human NK cell line KHYG-1, which does not express CD16a. FIG. 21B shows binding to KHYG-1 cells transduced to express the high affinity 158V variant of CD16a (KHYG-1-CD16aV). Each point and error bars represent mean and SD, respectively, of fold-over-background of median fluorescence intensity signals from duplicate wells.

FIGS. 22A-22D are graphs showing ligand blocking assessed by surface plasmon resonance (SPR) by injecting recombinant human epidermal growth factor (EGF) over recombinant human epidermal growth factor receptor (EGFR) that was first bound to another binding agent. FIG. 22A shows ligand blocking of EGF to captured EGFR-TriNKET. FIG. 22B shows ligand blocking of EGF to captured panitumumab. FIG. 22C shows ligand blocking of EGF to captured cetuximab. FIG. 22D shows binding of EGF to free EGFR captured via His-Tag. Smaller inset panels zoom in on the EGF binding stage of experiment.

FIGS. 23A-23B are graphs showing inhibition of EGF-induced EGFR phosphorylation in tumor cell lines. FIG. 23A shows inhibition of EGFR phosphorylation in NCI-H292-NucLight Green (lung carcinoma) cells. FIG. 23B shows inhibition of EGFR phosphorylation in FaDu (head and neck squamous cell carcinoma [HNSCC], hypopharyngeal squamous cell carcinoma subset) cells. Each point and error bars represent mean and SD, respectively, of percent inhibition calculated from duplicate wells.

FIGS. 24A-24B are graphs showing tumor cell growth inhibition by EGFR-TriNKET, cetuximab, or panitumumab observed over the course of 72 hours. FIG. 24A shows inhibition of proliferation of NCI-H292-NucLight Green cells. FIG. 24B shows inhibition of proliferation of FaDu cells.

FIGS. 25A-25C are graphs showing short-term lysis of EGFR-expressing tumor cell lines measured in co-culture with overnight-rested primary human NK cells from a donor with a V/F CD16a genotype. E:T-only (no treatment) background lysis is marked with a dotted line. FIG. 25A shows lysis of Detroit 562 (pharyngeal carcinoma) cells. FIG. 25B shows lysis of NCI-H1975 (non-small-cell lung cancer [NSCLC] adenocarcinoma) cells. FIG. 25C shows lysis of HT29 cells. Each point and error bars represent mean and SD, respectively, of specific lysis from triplicate co-culture wells.

FIGS. 26A-26D are graphs showing long-term lysis of EGFR-expressing tumor cell lines measured in co-culture with overnight-rested NK cells over 72 hours in the presence of 50% pooled human serum. FIG. 26A shows lysis of 786-O (renal carcinoma) cells in co-culture with NK cells with only a low-affinity CD16a variant (158FF or F/F). FIG. 26B shows lysis of 786-O cells in co-culture with NK cells with some presence of high-affinity CD16a polymorphism F158V (158VF or V/F). FIG. 26C shows lysis of NCI-H1975 cells in co-culture with NK cells with only a low-affinity CD16a variant (158FF or F/F). FIG. 26D shows lysis of NCI-H1975 cells in co-culture with NK cells with some presence of high-affinity CD16a polymorphism F158V (158VF or V/F). Each point and error bars represent mean and SD, respectively, of percent inhibition from 8 images total read out from duplicate co-culture wells with a different test article.

FIG. 27 is a graph showing lysis of 786-O renal carcinoma cells measured in co-culture with overnight-rested primary human NK cells. Each point and error bars represent mean and SD, respectively, of specific lysis from triplicate co-culture wells with a different test article.

FIGS. 28A-28B are graphs showing degranulation and cytokine production by human NK cells in human peripheral blood mononuclear cell (PBMC) co-cultures with tumor cells. FIG. 28A shows degranulation and cytokine production by human NK cells in co-culture with 786-O (renal cell carcinoma) target cells at a 4:1 effector-to-target (E:T) ratio. FIG. 28B shows degranulation and cytokine production by human NK cells in co-culture with NCI-H1975 target cells at a 2:1 E:T. Each point and error bars represent mean and SD, respectively, of the proportion of NK cells actively degranulating and producing cytokine from duplicate co-culture wells with a different test article. Where non-zero, E:T no treatment background activation is marked with a dotted line.

FIGS. 29A-29B are graphs showing release of interferon gamma (IFNγ) from human NK cells measured in the presence tumor cells. FIG. 29A shows release of IFNγ from human NK cells in the presence of NCI-H1975 cells. FIG. 29B shows release of IFNγ from human NK cells in the presence of HT29 cells. Each point and error bars represent mean and SD, respectively, concentrations of IFNγ from duplicate co-culture wells with a different test article. E:T no treatment background IFNγ content is marked with a dotted line.

FIGS. 30A-30B are graphs showing induction of programmed death-ligand 1 (PD-L1) on tumor cells measured after co-culture with human NK cells. FIG. 30A shows PD-L1 induction on NCI-H1975 cells. FIG. 30B shows PD-L1 induction on HT29 cells. Each point and error bars represent mean and SD, respectively, of A PD-L1 MFI from duplicate co-culture wells with a different test article.

FIGS. 31A-31B are graphs showing lysis of tumor cells was measured in co-culture with primed primary human CD8+ T cells. E:T no treatment background lysis is marked with a dotted line. FIG. 31A shows lysis of 786-O cells. FIG. 31B shows lysis of NCI-H1975 cells. Each point and error bars represent mean and SD, respectively, of specific lysis from triplicate co-culture wells with a different test article.

FIGS. 32A-32C are graphs showing lysis of cells measured in the presence of human complement serum. Basal lysis with serum but no additional treatment is marked with a dotted line. FIG. 32A shows lysis of 786-O cells. FIG. 32B shows lysis of KYSE-270 (esophageal squamous cell carcinoma) cells. FIG. 32B shows lysis of Raji (Burkitt's lymphoma) cells. Each point and error bars represent mean and SD, respectively, of specific lysis from triplicate culture wells with a different test article.

FIG. 33 is a graph showing phagocytosis by MO macrophages in co-culture with 786-O cells over 2 hours in the presence of 50% pooled human serum. Each point and error bars represent mean and SD, respectively, of percent phagocytosis from duplicate co-culture wells with a different test article.

FIGS. 34A-34F are graphs showing tumor volumes in nude mice engrafted with 4×10⁶ NCI-H292 cells and dosed intraperitoneally (IP) with hIgG1 isotype, EGFR-TriNKET, or cetuximab equimolar to 20 μg (1 mg/kg) or 100 μg (5 mg/kg) of EGFR-TriNKET. FIG. 34A shows individual tumor volumes at indicated days with administration of 100 μg (5 mg/kg) of EGFR-TriNKET or isotype control at days indicated by vertical dashed lines. FIG. 34B shows individual tumor volumes at indicated days with administration of cetuximab equimolar to 100 μg (5 mg/kg) of EGFR-TriNKET or isotype control at days indicated by vertical dashed lines. FIG. 34C shows group tumor volumes as mean±standard error of the mean (SEM) at indicated days with administration of 100 μg (5 mg/kg) of EGFR-TriNKET, equimolar cetuximab, or isotype control at days indicated by vertical dashed lines. FIG. 34D shows individual tumor volumes at indicated days with administration of 20 μg (1 mg/kg) of EGFR-TriNKET or isotype control at days indicated by vertical dashed lines. FIG. 34E shows individual tumor volumes at indicated days with administration of cetuximab equimolar to 20 μg (1 mg/kg) of EGFR-TriNKET or isotype control at days indicated by vertical dashed lines. FIG. 34F shows group tumor volumes as mean±SEM at indicated days with administration of 20 μg (1 mg/kg) of EGFR-TriNKET, cetuximab, or isotype control at days indicated by vertical dashed lines.

FIGS. 35A-35D are graphs showing tumor volumes in nude mice engrafted with 4×10⁶ NCI-H292 cells and dosed IP with indicated treatment. FIG. 35A shows tumor volumes of mice dosed with EGFR-TriNKET or hIgG1 isotype control on days indicated by vertical dashed line. FIG. 35B shows tumor volumes of mice dosed with EGFR-TriNKET-FcγRsi or hIgG1 isotype control on days indicated by vertical dashed line. FIG. 35C shows tumor volumes of mice dosed with EGFR-TriNKET or hIgG1 isotype control in combination with NK cell depletion (NK depl) on days indicated by vertical dashed line. FIG. 35D shows group tumor volumes from FIGS. 35A-35C as mean±SEM.

FIGS. 36A-36I are graphs showing tumor volumes in nude mice (n=6/group) engrafted with 2×10⁶ FaDu cells and dosed IP with hIgG1 isotype, EGFR-TriNKET, or cetuximab equimolar to 4 μg (0.2 mg/kg), 20 μg (1 mg/kg), or 100 μg (5 mg/kg) of EGFR-TriNKET. FIG. 36A shows individual tumor volumes with administration of 100 μg (5 mg/kg) of EGFR-TriNKET or isotype control at days indicated by vertical dashed lines. FIG. 36B shows individual tumor volumes at indicated days with administration of cetuximab equimolar to 100 μg (5 mg/kg) of EGFR-TriNKET or isotype control at days indicated by vertical dashed lines. FIG. 36C shows group tumor volumes as mean±SEM at indicated days with administration of 100 μg (5 mg/kg) of EGFR-TriNKET, equimolar cetuximab, or isotype control at days indicated by vertical dashed lines. FIG. 36D shows individual tumor volumes at indicated days with administration of 20 μg (1 mg/kg) of EGFR-TriNKET or isotype control at days indicated by vertical dashed lines. FIG. 36E shows individual tumor volumes at indicated days with administration of cetuximab equimolar to 20 μg (1 mg/kg) of EGFR-TriNKET or isotype control at days indicated by vertical dashed lines. FIG. 36F shows group tumor volumes as mean±SEM at indicated days with administration of 20 μg (1 mg/kg) of EGFR-TriNKET, cetuximab, or isotype control at days indicated by vertical dashed lines. FIG. 36G shows individual tumor volumes at indicated days with administration of 4 μg (0.2 mg/kg) of EGFR-TriNKET or isotype control at days indicated by vertical dashed lines. FIG. 36H shows individual tumor volumes at indicated days with administration of cetuximab equimolar to 4 μg (0.2 mg/kg) of EGFR-TriNKET or isotype control at days indicated by vertical dashed lines. FIG. 36I shows group tumor volumes as mean±SEM at indicated days with administration of 4 μg (0.2 mg/kg) of EGFR-TriNKET, cetuximab, or isotype control at days indicated by vertical dashed lines.

FIGS. 37A-37B are graphs showing binding of Tyrp1-TriNKET and TA99 parent monoclonal antibody (mAb) to indicated cells. FIG. 37A shows binding to B16F10 (melanoma) cells. FIG. 37B shows binding to CT26-Tyrp1 (colorectal carcinoma) cells.

FIGS. 38A-38B are graphs showing surface staining of selected antigens on mouse NK cells purified from spleen and cultured with B16F10 cells at a 1:1 effector-to-target (E:T) ratio. Cells were assessed in the NK cell gate (NK1.1⁺/CD3⁻). FIG. 38A shows % CD107a positivity of cells following incubation with indicated proteins. FIG. 38B shows % IFNγ positivity of cells following incubation with indicated proteins.

FIGS. 39A-39D are graphs showing analysis of C57BL/6 mice (n=10/group) engrafted with 2×10⁵ B16F10 cells and dosed IP with mIgG2a isotype, Tyrp1-TriNKET, or TA99 (parental mAb) at 100 μg/dose. FIG. 39A shows individual tumor volumes with administration of 100 μg of Tryp1-TriNKET or isotype control at days indicated by vertical dashed lines. FIG. 39B shows individual tumor volumes with administration of 100 μg TA99 or isotype control at days indicated by vertical dashed lines. FIG. 39C shows group tumor volumes as mean±SEM at indicated days with administration of 100 μg of Tryp1-TriNKET, TA99, or isotype control at days indicated by vertical dashed lines. FIG. 39D shows Kaplan-Meier survival curves of mice from FIGS. 39A-39C.

FIGS. 40A-40D are graphs showing analysis of Balb/c mice (n=10/group) engrafted with 5×10⁵ CT26-Tyrp1 cells and dosed IP with mIgG2a isotype, Tyrp1-TriNKET, or TA99 (parental mAb) at 100 μg/dose. FIG. 40A shows individual tumor volumes with administration of 100 μg of Tryp1-TriNKET or isotype control at days indicated by vertical dashed lines. FIG. 40B shows individual tumor volumes with administration of 100 μg TA99 or isotype control at days indicated by vertical dashed lines. FIG. 40C shows group tumor volumes as mean±SEM at indicated days with administration of 100 μg of Tryp1-TriNKET, TA99, or isotype control at days indicated by vertical dashed lines. FIG. 40D shows Kaplan-Meier survival curves of mice from FIGS. 40A-40C.

FIGS. 41A-41B are graphs showing analysis of C57BL/6 mice (n=8-9/group) inoculated subcutaneously (SC) with 2×10⁵ B16F10 cells and dosed IP with 150-μg isotype or Tyrp1-TriNKET. FIG. 41A shows quantification of tumor-infiltrating NK cells, and CD4 and CD8 T cells (# of cells/gram tumor). FIG. 41B shows frequencies of different immune populations within the total CD45+ leukocyte population (left) and # of cells/gram tumor (right). Data shown are mean±standard error of the mean (SEM).

FIGS. 42A-42C are graphs showing quantification of immune cells that express LAG-3 (lymphocyte activation gene-3), PD-1 (programmed cell death-1), TIGIT (T-cell immunoglobulin and ITIM domain), or TIM-3 (T-cell immunoglobulin and mucin domain-3) in B16F10 tumor tissues from mice 7 days after treatment with 150-gg isotype or Tyrpl-TriNKET. FIG. 42A shows quantification of tumor-infiltrating NK cells. FIG. 42B shows quantification of tumor-infiltrating CD8+ T cells. FIG. 42C shows quantification of tumor-infiltrating CD4+ T cells.

FIGS. 43A-43D are graphs showing C57BL/6 mice (n=10/group) engrafted with 2×10⁵ B16F10 cells and dosed IP with 100-μg isotype, 100-pg Tyrp1-TriNKET, 200-μg anti-PD-1, or the combination of Tyrp1-TriNKET and anti-PD-1. FIG. 43A shows individual tumor volumes with administration of Tryp1-TriNKET or isotype control at days indicated by vertical dashed lines. FIG. 43B shows individual tumor volumes with administration of 200 μg anti-PD-1 or isotype control at days indicated by vertical dashed lines. FIG. 43C shows individual tumor volumes with administration of 100 μg Tryp1-TriNKET and 200 μg anti-PD-1 or isotype control at days indicated by vertical dashed lines. FIG. 43D shows Kaplan-Meier survival curves of mice from FIGS. 43A-43C.

FIG. 44 is a graph showing enumeration of different immune cell subsets in whole blood following incubation with 450 nM of EGFR-TriNKET, cetuximab, or rituximav for 24 hours for 3 healthy human donors. Each point represents the mean of 4 replicate-normalized counts for a given immune cell subset in whole blood from a different donor. Mean and SD across 3 donors are denoted with dashes and error bars, respectively.

FIG. 45 is a graph showing degranulation and cytokine production by human NK cells among PBMCs in the absence of EGFR-expressing target cells co-cultured with 4 nM of EGFR-TriNKET, cetuximab, or a TriNKET isotype control. Each point represents the mean of the proportion of NK cells actively degranulating and producing cytokine from duplicate co-culture wells. Mean and SD across 3 donors are denoted with dashes and error bars, respectively.

FIGS. 46A-46F are graphs showing inflammatory cytokine production by human PBMCs detected via a Meso Scale Discovery (MSD) assay of culture supernatant. FIG. 46A shows production of IFNγ. FIG. 46B shows production of interleukin (IL)-1β. FIG. 46C shows production of IL-2. FIG. 46D shows production of IL-6. FIG. 46E shows production of IL-10. FIG. 46F shows production of TNFα. Data are summarized across 3 healthy human donors, with mean values across donors and SD marked with a dash and error bars, respectively. * indicates that IFNγ and TNFα release induced by CD3 engagement was so significant across all donors that corresponding supernatants yielded signals in excess of the upper limit of detection (ULOD) of the assay, in some cases by over an order of magnitude.

FIG. 47 is a graph showing inhibition of EGF-induced EGFR phosphorylation assessed using primary normal human epidermal keratinocytes. Each point and error bars represent mean and SD of percent inhibition from duplicate wells, respectively.

FIG. 48 is a graph showing inhibition of the growth of EGFR+ normal human epidermal keratinocytes observed over the course of 72 hours. Each point and error bars represent mean and SD, respectively, of % growth inhibition from 8 images total read out from duplicate culture wells with a different test article.

FIGS. 49A-49B are graphs showing NK cell selectivity for tumor or normal epidermal growth factor receptor-expressing (EGFR⁺) target cells. FIG. 49A shows inhibition of HT29 tumor cells. FIG. 49B shows inhibition of primary human normal prostate epithelial cells (PrECs). Each point and error bars represent mean and SD from duplicate co-culture wells with a different test article.

FIG. 50 is a graph showing CD69 upregulation on NK cells cultured with HT29 tumor cells or PrECs after a 72-hour incubation. Each data point represents mean CD69 MFI on NK cells from a different donor. Mean values across donors for a given cell type and SD are marked with a dash and error bars, respectively.

FIG. 51 is a graph showing dose-responsive binding of EGFR-TriNKET and cetuximab on primary cynomolgus monkey esophageal epithelial cells. Each point and error bars represent mean and SD, respectively, of fold over background of median fluorescence intensity signals from duplicate wells for a single donor animal.

FIGS. 52A-52B are graphs showing binding of EGFR-TriNKET, cetuximab, an hIgG1 isotype control, and a TriNKET isotype-FcγRsi to cynomolgus monkey immune cell subsets. FIG. 52A shows binding to immune cells in isolated PBMCs. FIG. 52B shows binding to immune cells in whole blood. Each point represents the mean number of molecules bound per cell of a given immune subset for a single animal calculated from duplicate wells. Dashes and error bars represent mean and SD, respectively.

FIG. 53 is a graph showing enumeration of different immune cell subsets in cynomolgus monkey whole blood following incubation with 450 nM of EGFR-TriNKET, cetuximab, or rituximab for 24 hours. Each point represents the mean of 4 replicate normalized counts for a given immune cell subset in whole blood from a different animal. Mean and SD across 3 animals are denoted with dashes and error bars, respectively.

FIG. 54 is a graph showing degranulation and cytokine production by cynomolgus monkey NK cells among PBMCs in the absence of EGFR-expressing target cells. Each point represents the mean of the proportion of NK cells actively degranulating and producing cytokines from duplicate co-culture wells. Mean and SD across 3 animals are denoted with dashes and error bars, respectively.

FIGS. 55A-55B are graphs showing degranulation and cytokine production by cynomolgus monkey NK cells in peripheral blood mononuclear cell (PBMC) co-cultures with tumor target cells. FIG. 55A shows degranulation and cytokine production by cynomolgus monkey NK cells co-cultured with 786-O cells at a 4:1 E:T ratio. FIG. 55B shows degranulation and cytokine production by cynomolgus monkey NK cells co-cultured with NCI-H1975 cells at a 2:1 E:T. Each point and error bars represent mean and SD, respectively. Where nonzero, E:T no treatment background activation is marked with a dotted line.

FIG. 56 is a schematic of an exemplary purification process of EGFR-TriNKET according to an embodiment.

FIGS. 57A-57C are Kaplan-Meier survival curves of athymic nude mice (n=6/group) subcutaneously inoculated with FaDu (head and neck squamous cell carcinoma [HNSCC], hypopharyngeal squamous cell carcinoma subset) cell tumors and dosed intraperitoneally with hIgG1 isotype, EGFR-TriNKET, or cetuximab equimolar to 100 μg (5 mg/kg) (FIG. 57A), 20 μg (1 mg/kg) (FIG. 57B), or 4 μg (0.2 mg/kg) (FIG. 57C) of EGFR-TriNKET.

### DETAILED DESCRIPTION

The present application provides pharmaceutical formulations including a multi-specific binding protein having an EGFR-binding scFv, an NKG2D-binding Fab, and an antibody Fc domain, and ingredients in the formulation optimized for stability of the multi-specific binding protein. Also provided are therapeutic uses of the multi-specific binding protein and pharmaceutical formulation thereof, for example for treating cancer. The multi-specific binding proteins are capable of binding EGFR on a cancer cell and NKG2D and CD16 on natural killer cells. Such binding brings the cancer cell into proximity with the natural killer cell, which facilitates direct and indirect destruction of the cancer cell by the natural killer cells. Various aspects of the formulations and the multi-specific binding proteins thereof described in present application are set forth below in sections; however, aspects of the multi-specific binding proteins described in one particular section are not to be limited to any particular section.

Also provided are methods of treating specific types of cancer in a subject in need thereof, or treating cancer in target patient populations—either as a monotherapy of the multi-specific binding proteins described in the present disclosure, or a combination therapy with one or more other therapeutic agents. In some embodiments, a method described in the present disclosure involves prior treatment of a patient with a therapeutic for treatment of cancer or for prophylactic treatment or management of a condition or effect known to be associated with or an effect of the cancer treatment. In some embodiments, the present disclosure provides treatment of unresectable, recurrent, or metastatic tumor/cancer (e.g., a solid tumor). In some embodiments, the cancer is an advanced solid tumor for which there is no effective standard therapy or the cancer patient is intolerant to standard/current cancer therapies.

To facilitate an understanding of the present application, a number of terms and phrases are defined below.

The terms “a” and “an” as used herein mean “one or more” and include the plural unless the context is inappropriate.

As used herein, the term “antigen-binding site” refers to the part of the immunoglobulin molecule that participates in antigen binding. In human antibodies, the antigen-binding site is formed by amino acid residues of the N-terminal variable (“V”) regions of the heavy (“H”) and light (“L”) chains. Three highly divergent stretches within the V regions of the heavy and light chains are referred to as “hypervariable regions” which are interposed between more conserved flanking stretches known as “framework regions,” or “FR.” Thus the term “FR” refers to amino acid sequences which are naturally found between and adjacent to hypervariable regions in immunoglobulins. In a human antibody molecule, the three hypervariable regions of a light chain and the three hypervariable regions of a heavy chain are disposed relative to each other in three-dimensional space to form an antigen-binding surface. The antigen-binding surface is complementary to the three-dimensional surface of a bound antigen, and the three hypervariable regions of each of the heavy and light chains are referred to as “complementarity-determining regions,” or “CDRs.” In certain animals, such as camels and cartilaginous fish, the antigen-binding site is formed by a single antibody chain providing a “single domain antibody.” Antigen-binding sites can exist in an intact antibody, in an antigen-binding fragment of an antibody that retains the antigen-binding surface, or in a recombinant polypeptide such as an scFv, using a peptide linker to connect the heavy chain variable domain to the light chain variable domain in a single polypeptide.

The term “tumor-associated antigen” as used herein means any antigen including but not limited to a protein, glycoprotein, ganglioside, carbohydrate, lipid that is associated with cancer. Such antigen can be expressed on malignant cells or in the tumor microenvironment such as on tumor-associated blood vessels, extracellular matrix, mesenchymal stroma, or immune infiltrates. In certain embodiments of the present disclosure, the term “tumor-associated antigen” refers to EGFR.

As used herein, the terms “subject” and “patient” refer to an organism to be treated by the methods and compositions described herein. Such organisms preferably include, but are not limited to, mammals (e.g., murines, simians, equines, bovines, porcines, canines, felines, and the like), and more preferably include humans.

As used herein, the term “effective amount” refers to the amount of a compound (e.g., a compound described in the present application) sufficient to effect beneficial or desired results. An effective amount can be administered in one or more administrations, applications or dosages and is not intended to be limited to a particular formulation or administration route. As used herein, the term “treating” includes any effect, e.g., lessening, reducing, modulating, ameliorating or eliminating, that results in the improvement of the condition, disease, disorder, and the like, or ameliorating a symptom thereof.

The term “about” refers to any minimal alteration, not limited to experimental variations/errors, in the concentration or amount of an agent that does not change the efficacy of the agent in preparation of a formulation and in treatment of a disease or disorder. In certain embodiments, the term “about” may include ±5%, ±10%, or ±15% of a specified numerical value or data point. In some embodiments, the variation of ±5%, ±10%, or ±15% may be beyond the ranges of experimental variations/errors.

Ranges can be expressed in this disclosure as from “about” one particular value, and/or to “about” another particular value. When such a range is expressed, another aspect includes from the one particular value and/or to the other particular value. Similarly, when values are expressed as approximations, by use of the antecedent “about,” it is understood that the particular value forms another aspect. It is further understood that the endpoints of each of the ranges are significant both in relation to the other endpoint, and independently of the other endpoint. It is also understood that there are a number of values disclosed in this disclosure, and that each value is also disclosed as “about” that particular value in addition to the value itself. It is also understood that throughout the application, data are provided in a number of different formats and that this data represent endpoints and starting points and ranges for any combination of the data points. For example, if a particular data point “10” and a particular data point “15” are disclosed, it is understood that greater than, greater than or equal to, less than, or equal to, and equal to 10 and 15 are considered disclosed as well as between 10 and 15. It is also understood that each unit between two particular units is also disclosed. For example, if 10 and 15 are disclosed, then 11, 12, 13, and 14 are also disclosed.

As used herein, the term “anti-EGFR therapeutics” refers to EGFR inhibitors, e.g., an anti-EGFR multi-specific protein, anti-EGFR antibody. In some embodiments, the EGFR therapeutic is a monoclonal antibody. Without being bound by mechanism of theory, an anti-EGFR monoclonal antibody binds specifically to the extracellular domain of the EGFR, competitively inhibiting binding of other growth factor ligands, including the epidermal growth factor (EGF) and transforming growth factor-alpha.

Non-limiting examples of anti-EGFR therapeutics include cetuximab, panitumumab, and necitumumab.

As used herein, the term “standard therapy” refers to therapy considered to be the current best practice or standard management for the treatment of certain diseases. Standard therapies are established by medical or biopharmaceutical authorities, such as official health care providers or national or regional agencies (e.g., U.S. Food and Drug Administration).

As used herein, the term “pharmaceutical formulation” refers to the combination of an active agent (e.g., a multi-specific binding protein) with a carrier, inert or active, making the agent especially suitable for diagnostic or therapeutic use in vivo or ex vivo.

As used herein, EGFR (also known as epidermal growth factor receptor, ErbB-1, or HER1 in humans) refers to the protein of Uniprot Accession No. P00533 (human) and related isoforms and orthologs.

Throughout the description, where compositions are described as having, including, or comprising specific components, or where processes and methods are described as having, including, or comprising specific steps, it is contemplated that, additionally, there are compositions that consist essentially of, or consist of, the recited components, and that there are processes and methods according to the present application that consist essentially of, or consist of, the recited processing steps.

As a general matter, compositions specifying a percentage are by weight unless otherwise specified. Further, if a variable is not accompanied by a definition, then the previous definition of the variable controls.

### I. Multi-Specific Binding Proteins for Use in Treating Cancer, and Preparation of Pharmaceutical Formulations

The present disclosure provides multi-specific binding proteins having an antigen-binding site that binds a tumor-associated antigen such as EGFR; an antigen-binding site that binds NKG2D; and an antibody Fc domain or portion thereof sufficient to bind CD16, or an antigen-binding site that binds CD16, and pharmaceutical formulations thereof for use, for example, in methods of treating recurrent, relapsed, unresectable, and/or metastatic cancers; head and neck squamous cell carcinoma (HNSCC); relapsed or metastatic colorectal cancer (CRC); recurrent or progressive non-small-cell lung cancer (NSCLC) during or after platinum doublet-based chemotherapy; recurrent or progressive NSCLC within 6 months after completing platinum-based chemotherapy for local disease; esophageal adenocarcinoma; triple-negative breast cancer, renal cell carcinoma, gastric cancer, or pancreatic cancer. In some embodiments, cancer is treated by administering to a patient a multi-specific binding protein (or a pharmaceutical formulation thereof) having an antigen-binding site that binds a tumor-associated antigen such as EGFR; an antigen-binding site that binds NKG2D; and an antibody Fc domain or portion thereof sufficient to bind CD16, or an antigen-binding site that binds CD16, in combination with nivolumab. In some embodiments, cancer is treated by administering to a patient who is eligible for an anti-PD-1 or an anti-PD-L1 therapy for a malignancy of epithelial origin, or a patient with a cancer for which no standard therapy exists or standard therapy has failed to treat a malignancy of epithelial origin; or a patient pre-treated with an anti-PD-1 or an anti-PD-L1 therapy, with a multi-specific binding protein (or a pharmaceutical formulation thereof) having an antigen-binding site that binds a tumor-associated antigen such as EGFR; an antigen-binding site that binds NKG2D; and an antibody Fc domain or portion thereof sufficient to bind CD16, or an antigen-binding site that binds CD16, in combination with an anti-PD-1 or an anti-PD-L1 therapy.

In some embodiments the cancer is an advanced solid tumor for which no effective standard therapy is available. In some embodiments, the patient selected for treatment is intolerant of standard therapies. In some embodiments, a patient has been or is pre-treated with an anti-PD-1 or an anti-PD-L1 therapy. In some embodiments, a patient has recurrent or progressive disease during or after platinum doublet-based chemotherapy, or has recurrent or progressive disease within 6 months after completing platinum-based chemotherapy for local disease.

Multi-specific binding proteins disclosed herein include an antigen-binding site that is capable of binding to NKG2D, a receptor expressed on the surface of cells including, but not limited to, NK cells, γδ T cells and CD8⁺ αβ T cells. Upon NKG2D binding, the multi-specific binding proteins may block natural ligands, such as ULBP6 and MICA, from binding to NKG2D, thereby inhibiting NK cell activation. In some embodiments, the antigen-binding site that binds NKG2D includes a heavy chain variable domain (VH) and a light chain variable domain (VL).

Multi-specific binding proteins disclosed herein further include an antigen-binding site that is capable of binding EGFR. EGFR-expressing cells may be found in solid tumors, for example, in indications such as lung cancer, breast cancer, kidney cancer, colorectal cancer, gastric cancer, brain cancer, glioma, bladder cancer, head and neck cancer (e.g., head and neck squamous cell carcinoma (HNSCC)), bladder cancer, renal cell carcinoma, pancreatic cancer, non-small cell lung cancer, liver cancer, cervical cancer, ovarian cancer or prostate cancer. In some embodiments, the antigen-binding site that is capable of binding EGFR includes a VH and a VL. In some embodiments, the sequence of the VH or VL of an EGFR-TriNKET of the present disclosure is a modified or variant sequence of the VH or VL of panitumumab. In some embodiments, the sequences of the VH and VL of an EGFR-TriNKET of the present disclosure are modified or variant sequences of the panitumumab VH and VL and contribute to an increased stability and binding affinity to EGFR compared to panitumumab.

Multi-specific binding proteins disclosed herein also include an antibody Fc domain, or portion thereof; or an antigen-binding site that is capable of binding to CD16 (FcγRIII), expressed on the surface of cells such as NK cells, macrophages, neutrophils, eosinophils, mast cells, and follicular dendritic cells.

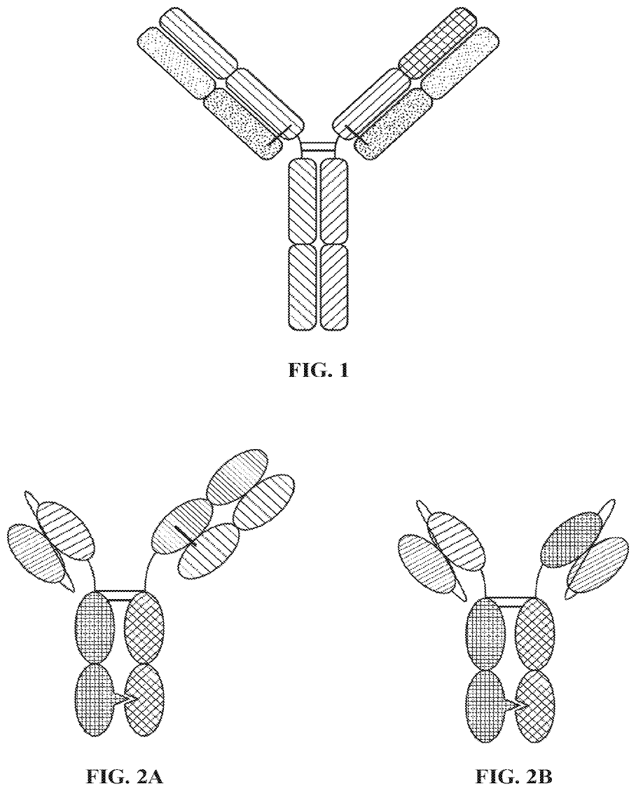

The multi-specific binding proteins described herein can take various formats. For example, one format is a heterodimeric, multi-specific antibody that includes a first immunoglobulin heavy chain, a first immunoglobulin light chain, a second immunoglobulin heavy chain and a second immunoglobulin light chain (FIG. 1). The first immunoglobulin heavy chain includes a first antibody Fc polypeptide (hinge-CH2-CH3), a first heavy chain variable domain (VH) and optionally a first heavy chain constant domain (CH1). The first immunoglobulin light chain includes a first light chain variable domain (VL) and optionally a first light chain constant domain (CL). The first immunoglobulin light chain, together with the first immunoglobulin heavy chain, forms an antigen-binding site that binds NKG2D. The second immunoglobulin heavy chain includes a second antibody Fc polypeptide (hinge-CH2-CH3), a second VH and optionally a second CH1. The second immunoglobulin light chain includes a second VL and optionally a second CL. The second immunoglobulin light chain, together with the second immunoglobulin heavy chain, forms an antigen-binding site that binds EGFR. In some embodiments, the first antibody Fc polypeptide and second antibody Fc polypeptide together are able to bind to CD16 (FIG. 1). In some embodiments, the first immunoglobulin light chain is identical to the second immunoglobulin light chain.

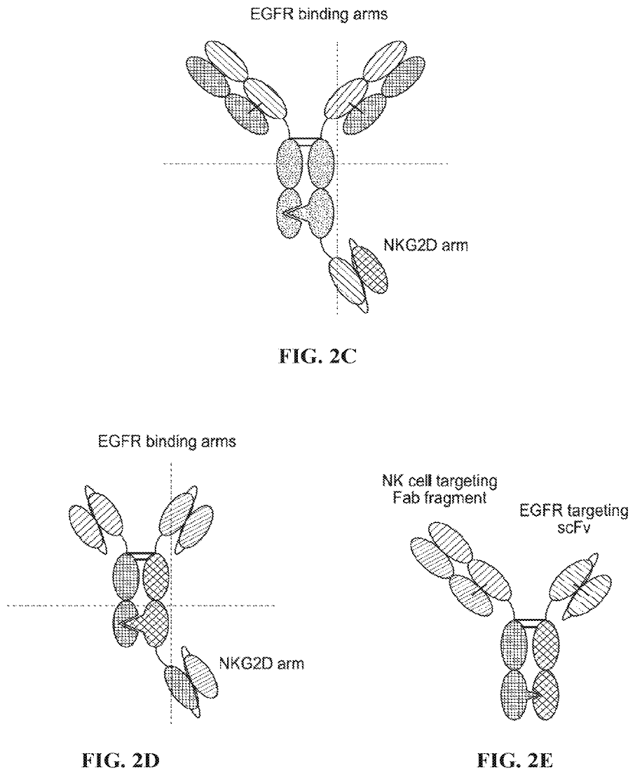

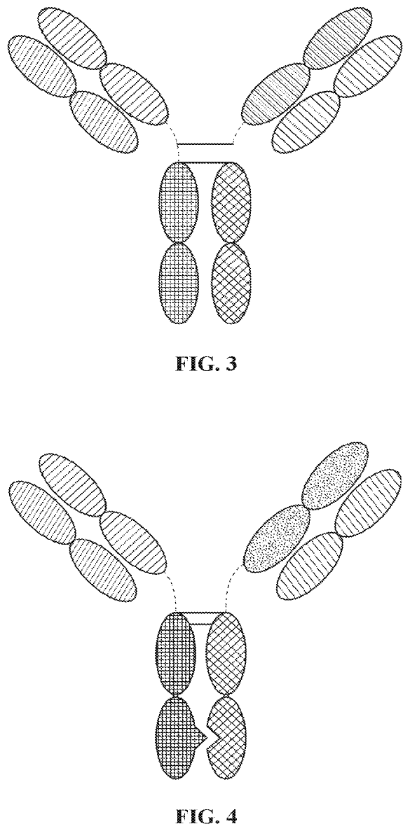

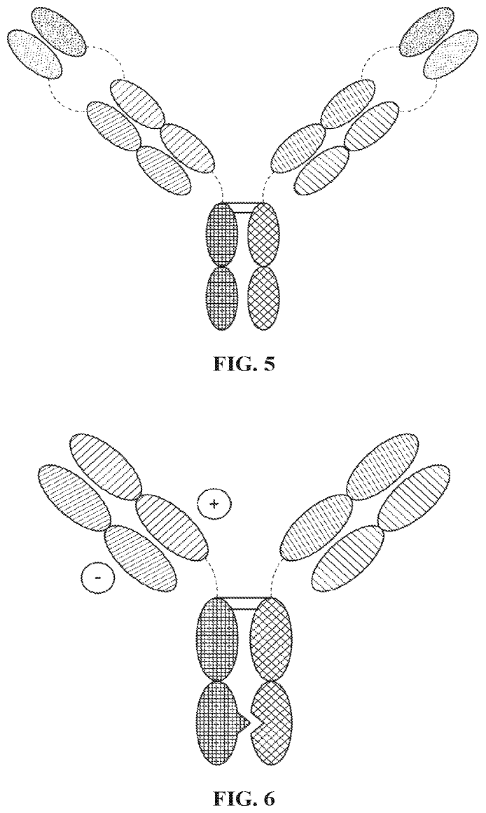

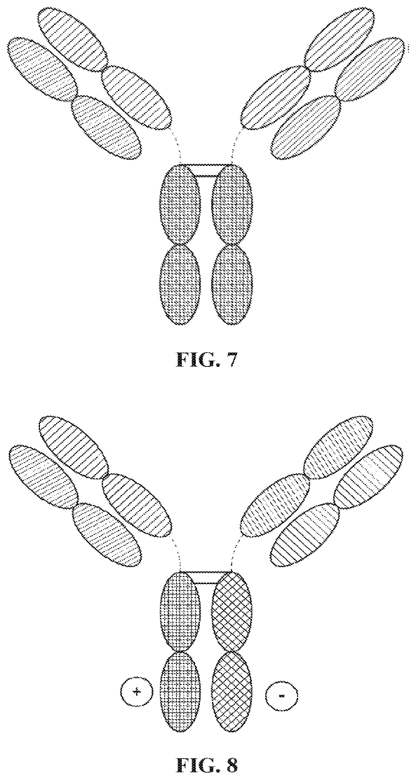

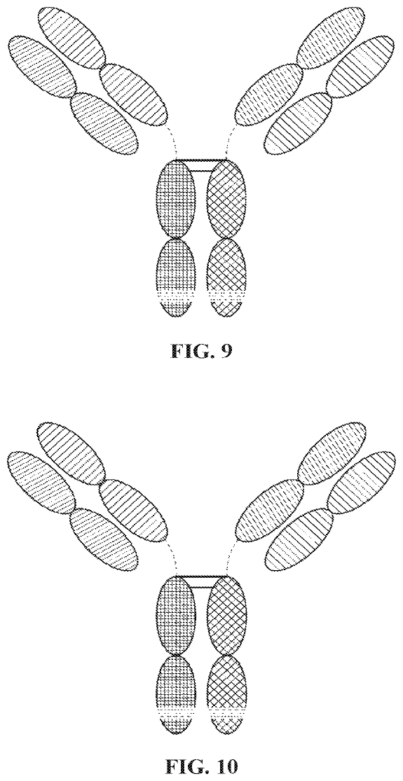

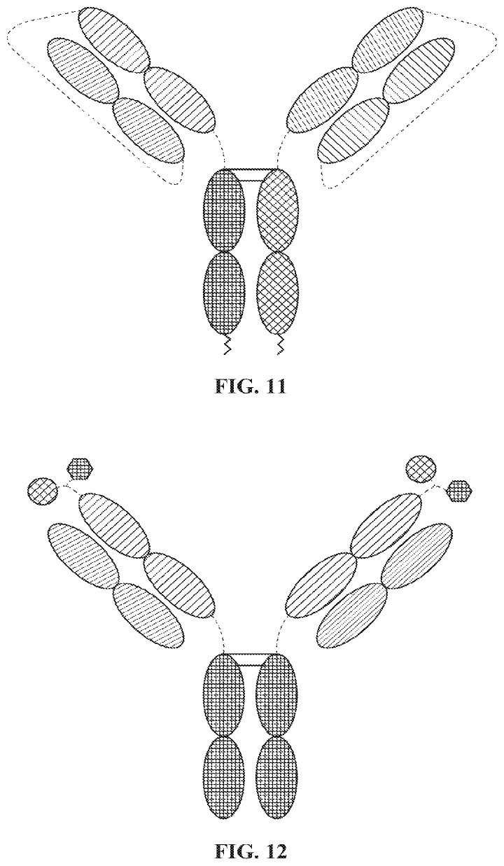

Another exemplary format involves a heterodimeric, multi-specific antibody including a first immunoglobulin heavy chain, a second immunoglobulin heavy chain and an immunoglobulin light chain (e.g., FIG. 2A). In some embodiments, the first immunoglobulin heavy chain includes a first antibody Fc polypeptide (hinge-CH2-CH3) fused via either a linker or an antibody hinge to a single-chain variable fragment (scFv) including a VH and VL, which together bind NKG2D or bind EGFR. In some embodiments, the second immunoglobulin heavy chain includes a second antibody Fc polypeptide (hinge-CH2-CH3), a second VH and a CH1. In some embodiments, the immunoglobulin light chain includes a VL and a CL. In some embodiments, the second immunoglobulin heavy chain pairs with the immunoglobulin light chain and binds to NKG2D or binds EGFR, with the proviso that when the first antibody Fc polypeptide is fused to an scFv that binds NKG2D, the second immunoglobulin heavy chain paired with the immunoglobulin light chain binds EGFR, but not NKG2D, and vice versa. In some embodiments, the scFv in the first immunoglobulin heavy chain binds EGFR; and the VH in the second immunoglobulin heavy chain and the VL in the immunoglobulin light chain, when paired, bind NKG2D (e.g., FIG. 2E). In some embodiments, the scFv in the first immunoglobulin heavy chain binds NKG2D; and the VH in the second immunoglobulin heavy chain and the VL in the immunoglobulin light chain, when paired, bind EGFR. In some embodiments, the first antibody Fc polypeptide and the second antibody Fc polypeptide together are able to bind to CD16 (e.g., FIG. 2A).

Another exemplary format includes a heterodimeric, multi-specific antibody including a first immunoglobulin heavy chain, and a second immunoglobulin heavy chain (e.g., FIG. 2B). In some embodiments, the first immunoglobulin heavy chain includes a first antibody Fc polypeptide (hinge-CH2-CH3) fused via either a linker or an antibody hinge to an scFv that includes a VH and VL, which together bind NKG2D, or bind EGFR. In some embodiments, the second immunoglobulin heavy chain includes a second antibody Fc polypeptide (hinge-CH2-CH3) fused via either a linker or an antibody hinge to an scFv including a second VH and a second VL, which together bind NKG2D, or EGFR, with the proviso that when the first antibody Fc polypeptide is fused to an scFv that binds NKG2D, the second antibody Fc polypeptide is fused to an scFv that binds EGFR, but not NKG2D, and vice versa. In some embodiments, the first antibody Fc polypeptide and the second antibody Fc polypeptide together are able to bind to CD16 (e.g., FIG. 2B).

In some embodiments, an scFv described above is linked to an antibody constant domain via a hinge sequence. In some embodiments, the hinge including amino acids Ala-Ser or Gly-Ser. In some embodiments, the hinge connecting an scFv (e.g., an scFv that binds EGFR or an scFv that binds NKG2D) and the antibody heavy chain constant domain including amino acids Ala-Ser. In some embodiments, the hinge connecting an scFv (e.g., an scFv that binds EGFR or an scFv that binds NKG2D) and the antibody heavy chain constant domain including amino acids Gly-Ser. In some other embodiments, the hinge including amino acids Ala-Ser and Thr-Lys-Gly. The hinge sequence can provide flexibility of binding to the target antigen, and balance between flexibility and optimal geometry.

An scFv described above includes a VH and a VL. In some embodiments, the heavy chain variable domain forms a disulfide bridge with the light chain variable domain to enhance stability of the scFv. In some embodiments, a disulfide bridge can be formed between the C44 residue of the VH and the C100 residue of the VL, the amino acid positions numbered under Kabat. In some embodiments, the cysteine residues at positions 44 of the VH and 100 of the VL are introduced by mutating the wild-type residues at these positions. In some embodiments, the VH is linked to the VL via a flexible linker. Any suitable linker can be used, for example, the (G₄S)₄ linker ((GlyGlyGlyGlySer)₄ (SEQ ID NO:119)). In some embodiments of the scFv, the VH is positioned at the N-terminus of the VL. In some embodiments of the scFv, the VH is positioned at the C terminus of the VL.

The multi-specific binding proteins described herein can also include one or more additional antigen-binding sites. The additional antigen-binding site(s) may be fused to the N-terminus of the constant region CH2 domain or to the C-terminus of the constant region CH3 domain, optionally via a linker sequence. In certain embodiments, the additional antigen-binding site(s) takes the form of an scFv that is optionally disulfide-stabilized, resulting in a tetravalent or trivalent multi-specific binding protein. For example, a multi-specific binding protein can include a first antigen-binding site that binds NKG2D, a second antigen-binding site that binds EGFR, an additional antigen-binding site that binds EGFR, and an antibody Fc domain a portion thereof sufficient to bind CD16, or a fourth antigen-binding site that binds CD16. Any one of these antigen-binding sites can either take the form of a Fab fragment or an scFv, such as an scFv described above.

In some embodiments, the additional antigen-binding site binds a different epitope of EGFR from the second antigen-binding site. In some embodiments, the additional antigen-binding site binds the same epitope of EGFR as the second antigen-binding site. In some embodiments, the additional antigen-binding site includes the same VH and VL complementarity determining region (CDR) sequences as the second antigen-binding site. In some embodiments, the additional antigen-binding site includes the same VH and VL sequences as the second antigen-binding site. In some embodiments, the additional antigen-binding site has the same amino acid sequence(s) as the second antigen-binding site. In some embodiments, the additional antigen-binding site includes VH and VL sequences that are different from the VH and VL sequences of the second antigen-binding site. In some embodiments, the additional antigen-binding site has an amino acid sequence that is different from the sequence of the second antigen-binding site. In some embodiments, the second antigen-binding site and the additional antigen-binding site bind different tumor-associated antigens. In some embodiments, the second antigen-binding site and the additional antigen-binding site bind different antigens. Exemplary formats are shown in FIG. 2C and FIG. 2D. Accordingly, in some embodiments the multi-specific binding proteins provide bivalent engagement of EGFR. Bivalent engagement of EGFR by the multi-specific binding proteins can stabilize EGFR on the tumor cell surface and enhance cytotoxicity of NK cells towards the tumor cells. Bivalent engagement of EGFR by the multi-specific binding proteins can confer stronger binding of the multi-specific binding proteins to the tumor cells, which may facilitate a stronger cytotoxic response of NK cells towards the tumor cells, especially towards tumor cells expressing a low level of EGFR.

The multi-specific binding proteins disclosed herein can take additional formats. In some embodiments, the multi-specific binding protein is in the Triomab form, which is a trifunctional, bispecific antibody that maintains an IgG-like shape. This chimera includes two half antibodies, each with one light and one heavy chain, that originate from two parental antibodies.

In some embodiments, the multi-specific binding protein is in a KiH form, which involves the knobs-into-holes (KiHs) technology. The KiH involves engineering C_{H}3 domains to create either a “knob” or a “hole” in each heavy chain to promote heterodimerization. The concept behind the “Knobs-into-Holes (KiH)” Fc technology was to introduce a “knob” in one CH3 domain (CH3A) by substitution of a small residue with a bulky one (e.g., T366W_{CH3A} in EU numbering). To accommodate the “knob,” a complementary “hole” surface was created on the other CH3 domain (CH3B) by replacing the closest neighboring residues to the knob with smaller ones (e.g., T366S/L368A/Y407V_{CH3B}). The “hole” mutation was optimized by structured-guided phage library screening (Atwell S, Ridgway J B, Wells J A, Carter P., Stable heterodimers from remodeling the domain interface of a homodimer using a phage display library, J. Mol. Biol. (1997) 270(1):26-35). X-ray crystal structures of KiH Fc variants (Elliott JM, Ultsch M, Lee J, Tong R, Takeda K, Spiess C, et al., Antiparallel conformation of knob and hole aglycosylated half-antibody homodimers is mediated by a CH2-CH3 hydrophobic interaction. J. Mol. Biol. (2014) 426(9):1947-57; Mimoto F, Kadono S, Katada H, Igawa T, Kamikawa T, Hattori K. Crystal structure of a novel asymmetrically engineered Fc variant with improved affinity for FcγRs. Mol. Immunol. (2014) 58(1):132-8) demonstrated that heterodimerization is thermodynamically favored by hydrophobic interactions driven by steric complementarity at the inter-CH3 domain core interface, whereas the knob-knob and the hole-hole interfaces do not favor homodimerization owing to steric hindrance and disruption of the favorable interactions, respectively.

In some embodiments, the multi-specific binding protein is in a dual-variable domain immunoglobulin (DVD-Ig™) form, which combines the target binding domains of two monoclonal antibodies via flexible naturally occurring linkers, and yields a tetravalent IgG-like molecule.

In some embodiments, the multi-specific binding protein is in an Orthogonal Fab interface (Ortho-Fab) form. In the ortho-Fab IgG approach (Lewis S M, Wu X, Pustilnik A, Sereno A, Huang F, Rick H L, et al., Generation of bispecific IgG antibodies by structure-based design of an orthogonal Fab interface. Nat. Biotechnol. (2014) 32(2):191-8), structure-based regional design introduces complementary mutations at the LC and HC_{VH-CH1} interface in only one Fab fragment, without any changes being made to the other Fab fragment.

In some embodiments, the multi-specific binding protein is in a 2-in-1 Ig format. In some embodiments, the multi-specific binding protein is in the ES form, which is a heterodimeric construct containing two different Fab fragments binding to targets 1 and target 2 fused to the Fc. Heterodimerization is ensured by electrostatic steering mutations in the Fc.

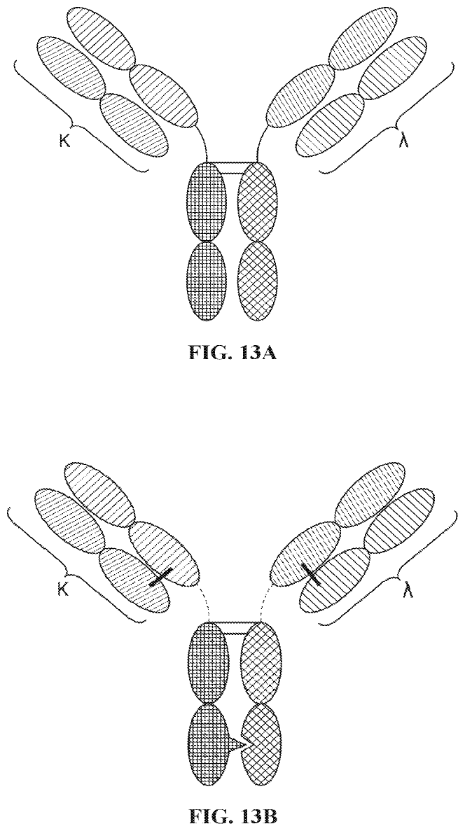

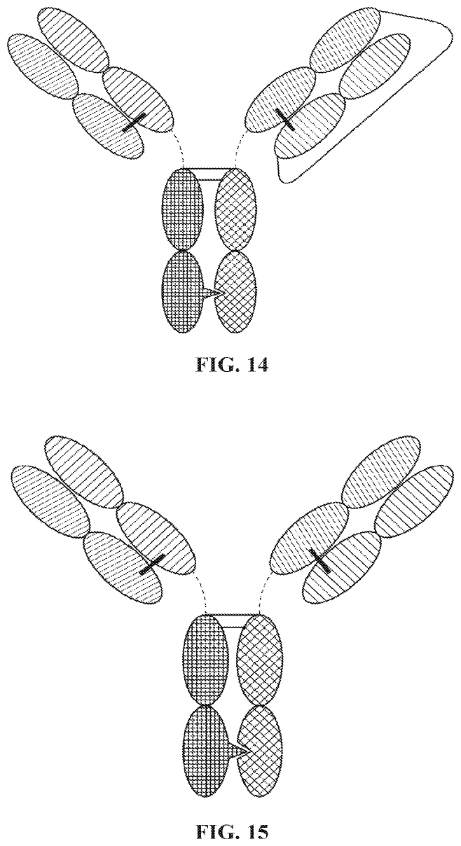

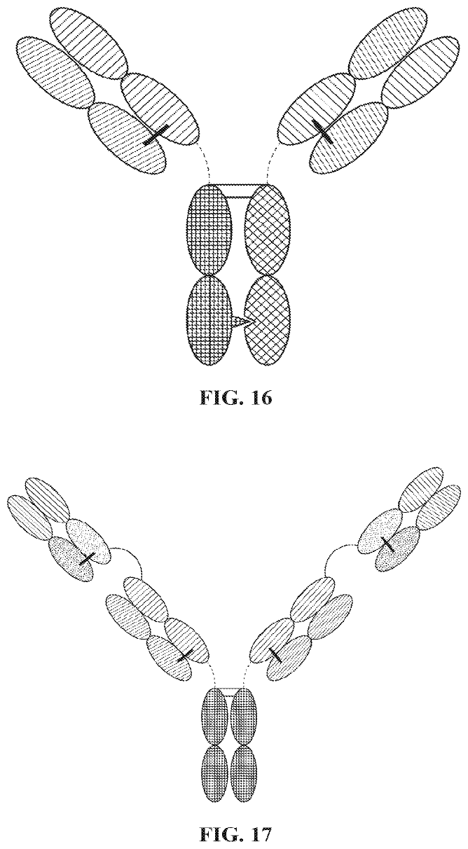

In some embodiments, the multi-specific binding protein is in a κλ-Body form, which is a heterodimeric construct with two different Fab fragments fused to an Fc stabilized by heterodimerization mutations: Fab fragment 1 targeting antigen 1 contains kappa LC, while Fab fragment 2 targeting antigen 2 contains lambda LC. FIG. 13A is an exemplary representation of one form of a κλ-Body; FIG. 13B is an exemplary representation of another κλ-Body.

In some embodiments, the multi-specific binding protein is in a Fab Arm Exchange form (antibodies that exchange Fab fragment arms by swapping a heavy chain and attached light chain (half-molecule) with a heavy-light chain pair from another molecule, which results in bispecific antibodies).

In some embodiments, the multi-specific binding protein is in a SEED Body form. The strand-exchange engineered domain (SEED) platform was designed to generate asymmetric and bispecific antibody-like molecules, a capability that expands therapeutic applications of natural antibodies. This protein engineering platform is based on exchanging structurally related sequences of immunoglobulin within the conserved CH3 domains. The SEED design allows efficient generation of AG/GA heterodimers, while disfavoring homodimerization of AG and GA SEED CH3 domains. (Muda M. et al., Protein Eng. Des. Sel. (2011, 24(5):447-54)).

In some embodiments, the multi-specific binding protein is in a LuZ-Y form, in which a leucine zipper is used to induce heterodimerization of two different HCs. (Wranik, B J. et al., J. Biol. Chem. (2012), 287:43331-9).

In some embodiments, the multi-specific binding protein is in a Cov-X-Body form. In bispecific CovX-Bodies, two different peptides are joined together using a branched azetidinone linker and fused to the scaffold antibody under mild conditions in a site-specific manner. Whereas the pharmacophores are responsible for functional activities, the antibody scaffold imparts long half-life and Ig-like distribution. The pharmacophores can be chemically optimized or replaced with other pharmacophores to generate optimized or unique bispecific antibodies. (Doppalapudi V R et al., PNAS (2010), 107(52);22611-22616).

In some embodiments, the multi-specific binding protein is in an OAsc-Fab heterodimeric form that includes a Fab fragment binding to target 1, and a scFab binding to target 2 fused to Fc. Heterodimerization is ensured by mutations in the Fc.

In some embodiments, the multi-specific binding protein is in a DuetMab form, which is a heterodimeric construct containing two different Fab fragments binding to antigens 1 and 2, and an Fc stabilized by heterodimerization mutations. Fab fragments 1 and 2 contain differential S-S bridges that ensure correct LC and HC pairing.

In some embodiments, the multi-specific binding protein is in a CrossmAb form, which is a heterodimeric construct with two different Fab fragments binding to targets 1 and 2, fused to an Fc stabilized by heterodimerization. CL and CH1 domains and VH and VL domains are switched, e.g., CH1 is fused in-frame with VL, while CL is fused in-frame with VH.

In some embodiments, the multi-specific binding protein is in a Fit-Ig form, which is a homodimeric construct where a Fab fragment binding to antigen 2 is fused to the N terminus of HC of a Fab fragment that binds to antigen 1. The construct contains wild-type Fc.

In some embodiments, the multi-specific binding protein includes: (i) a Fab including a VH and a VL that bind NKG2D; (ii) an scFv including a VH and a VL that bind EGFR; and (iii) an antibody Fc domain. In some embodiments, the VL of the scFv is linked to the VH of the scFv via a flexible linker. In some embodiments, the flexible linker includes or consists of the amino acid sequence of SEQ ID NO:119. In some embodiments, the VL of the scFv is positioned to the N-terminus of the VH of the scFv. In other embodiments, the VH of the scFv is positioned to the N-terminus of the VL of the scFv. In some embodiments, the VH of the scFv forms a disulfide bridge with the VL of the scFv. For example, in some embodiments, the disulfide bridge is formed between a cysteine residue (naturally present or introduced by mutation) at position 44 (C44) of the VH of the scFv and a cysteine residue (naturally present or introduced by mutation) at position 100 (C100) of the VL of the scFv, numbered under the Kabat numbering scheme. In some embodiments, the antibody Fc domain includes a first antibody Fc polypeptide linked to the Fab and a second antibody Fc polypeptide linked to the scFv. In some embodiments, the first antibody Fc polypeptide is linked to a heavy chain portion of the Fab. In some embodiments, the scFv is linked to the second antibody Fc polypeptide via a hinge including Ala-Ser or Gly-Ser. In some embodiments, the first and second antibody Fc polypeptides each include a hinge and a CH2 domain of a human antibody. In some embodiments, the human antibody is an IgG1 antibody. In some embodiments, the first and second antibody Fc polypeptides each include an amino acid sequence at least 90% (e.g., at least 90%, at least 91%, at least 92%, at least 93%, at least 94%, at least 95%, at least 96%, at least 97%, at least 98%, at least 99%, or 100%) identical to amino acids 234-332 of a wild-type human IgG1 antibody, numbered according to the EU index. In some embodiments, the first and second antibody Fc polypeptides each include different mutations promoting heterodimerization. For example, in some embodiments, the first antibody Fc polypeptide includes K360E and K409W substitutions and the second antibody Fc polypeptide includes Q347R, D399V, and F405T substitutions, numbered according to the EU index.

In some embodiments, the multi-specific binding protein includes: (i) a first antigen-binding site that binds NKG2D; (ii) a second antigen-binding site that binds EGFR; and (iii) an antibody Fe domain or a portion thereof sufficient to bind CD16, or a third antigen-binding site that binds CD16. In some embodiments, the first antigen-binding site includes a Fab and the second antigen-binding site includes an scFv that includes a VH and a VL. In some embodiments, the VL of the scFv is linked to the VH of the scFv via a flexible linker. In some embodiments, the flexible linker includes or consists of the amino acid sequence of SEQ ID NO:119. In some embodiments, the VL of the scFv is positioned to the N-terminus of the VH of the scFv. In other embodiments, the VH of the scFv is positioned to the N-terminus of the VL of the scFv. In some embodiments, the VH of the scFv forms a disulfide bridge with the VL of the scFv. For example, in some embodiments, the disulfide bridge is formed between a cysteine residue (naturally present or introduced by mutation) at position 44 (C44) of the VH of the scFv and a cysteine residue (naturally present or introduced by mutation) at position 100 (C100) of the VL of the scFv, numbered under the Kabat numbering scheme. In some embodiments, the antibody Fc domain includes a first antibody Fc polypeptide linked to the Fab and a second antibody Fc polypeptide linked to the scFv. In some embodiments, the first antibody Fc polypeptide is linked to a heavy chain portion of the Fab. In some embodiments, the scFv is linked to the second antibody Fc polypeptide via a hinge including Ala-Ser or Gly-Ser. In some embodiments, the first and second antibody Fc polypeptides each include a hinge and a CH2 domain of a human antibody. In some embodiments, the human antibody is an IgG1 antibody. In some embodiments, the first and second antibody Fc polypeptides each include an amino acid sequence at least 90% (e.g., at least 90%, at least 91%, at least 92%, at least 93%, at least 94%, at least 95%, at least 96%, at least 97%, at least 98%, at least 99%, or 100%) identical to amino acids 234-332 of a wild-type human IgG1 antibody, numbered according to the EU index. In some embodiments, the first and second antibody Fc polypeptides each incorporate different mutations promoting heterodimerization. For example, in some embodiments, the first antibody Fc polypeptide incorporates K360E and K409W substitutions and the second antibody Fc polypeptide incorporates Q347R, D399V, and F405T substitutions, numbered according to the EU index.

Individual components of the multi-specific binding proteins are described in more detail below.

### NKG2D-Binding Site

Upon binding to the NKG2D receptor and CD16 receptor on natural killer cells, and EGFR on cancer cells, the multi-specific binding proteins can engage more than one kind of NK-activating receptor, and may block the binding of natural ligands to NKG2D. In certain embodiments, the proteins can agonize NK cells in humans. In some embodiments, the proteins can agonize NK cells in humans and in other species such as rodents and cynomolgus monkeys. In some embodiments, the proteins can agonize NK cells in humans and in other species such as cynomolgus monkeys.

Table 1 lists exemplary peptide sequences of heavy chain variable domains and light chain variable domains that, in combination, can bind to NKG2D. In some embodiments, the heavy chain variable domain and the light chain variable domain are arranged in Fab format. In some embodiments, the heavy chain variable domain and the light chain variable domain are fused together to form an scFv.

The NKG2D binding sites listed in Table 1 can vary in their binding affinity to NKG2D, nevertheless, they all activate human NK cells.

Unless indicated otherwise, the CDR sequences provided in Table 1 are determined under Kabat numbering.

| | Heavy chain variable region | Light chain variable region |
|---|---|---|
| Clones | amino acid sequence | amino acid sequence |
| ADI-27705 | QVQLQQWGAGLLKPSETLSLTCAVYGG | DIQMTQSPSTLSASVGDRVTITCRAS |
| | SFSGYYWSWIRQPPGKGLEWIGEIDHSG | QSISSWLAWYQQKPGKAPKLLIYKA |
| | STNYNPSLKSRVTISVDTSKNQFSLKLSS | SSLESGVPSRFSGSGSGTEFTLTISSL |
| | VTAADTAVYYCARARGPWSFDPWGQG | QPDDFATYYCQQYNSYPITFGGGTK |
| | TLVTVSS | VEIK |
| | (SEQ ID NO: 1) | (SEQ ID NO: 5) |
| | CDR1 (SEQ ID NO: 2)-GSFSGYYWS | |
| | CDR2 (SEQ ID NO: 3)- | |
| | EIDHSGSTNYNPSLKS | |
| | CDR3 (SEQ ID NO: 4)- | |
| | ARARGPWSFDP | |
| ADI-27724 | QVQLQQWGAGLLKPSETLSLTCAVYGG | EIVLTQSPGTLSLSPGERATLSCRASQ |
| | SFSGYYWSWIRQPPGKGLEWIGEIDHSG | SVSSSYLAWYQQKPGQAPRLLIYGA |
| | STNYNPSLKSRVTISVDTSKNQFSLKLSS | SSRATGIPDRFSGSGSGTDFTLTISRL |
| | VTAADTAVYYCARARGPWSFDPWGQG | EPEDFAVYYCQQYGSSPITFGGGTK |
| | TLVTVSS | VEIK |
| | (SEQ ID NO: 1) | (SEQ ID NO: 6) |
| ADI-27740 | QVQLQQWGAGLLKPSETLSLTCAVYGG | DIQMTQSPSTLSASVGDRVTITCRAS |
| (A40) | SFSGYYWSWIRQPPGKGLEWIGEIDHSG | QSIGSWLAWYQQKPGKAPKLLIYKA |
| | STNYNPSLKSRVTISVDTSKNQFSLKLSS | SSLESGVPSRFSGSGSGTEFTLTISSL |
| | VTAADTAVYYCARARGPWSFDPWGQG | QPDDFATYYCQQYHSFYTFGGGTK |
| | TLVTVSS | VEIK |
| | (SEQ ID NO: 1) | (SEQ ID NO: 7) |
| ADI-27741 | QVQLQQWGAGLLKPSETLSLTCAVYGG | DIQMTQSPSTLSASVGDRVTITCRAS |
| | SFSGYYWSWIRQPPGKGLEWIGEIDHSG | QSIGSWLAWYQQKPGKAPKLLIYKA |
| | STNYNPSLKSRVTISVDTSKNQFSLKLSS | SSLESGVPSRFSGSGSGTEFTLTISSL |
| | VTAADTAVYYCARARGPWSFDPWGQG | QPDDFATYYCQQSNSYYTFGGGTK |
| | TLVTVSS | VEIK |
| | (SEQ ID NO: 1) | (SEQ ID NO: 8) |
| ADI-27743 | QVQLQQWGAGLLKPSETLSLTCAVYGG | DIQMTQSPSTLSASVGDRVTITCRAS |
| | SFSGYYWSWIRQPPGKGLEWIGEIDHSG | QSISSWLAWYQQKPGKAPKLLIYKA |
| | STNYNPSLKSRVTISVDTSKNQFSLKLSS | SSLESGVPSRFSGSGSGTEFTLTISSL |
| | VTAADTAVYYCARARGPWSFDPWGQG | QPDDFATYYCQQYNSYPTFGGGTK |
| | TLVTVSS | VEIK |
| | (SEQ ID NO: 1) | (SEQ ID NO: 9) |
| ADI-28153 | QVQLQQWGAGLLKPSETLSLTCAVYGG | ELQMTQSPSSLSASVGDRVTITCRTS |
| | SFSGYYWSWIRQPPGKGLEWIGEIDHSG | QSISSYLNWYQQKPGQPPKLLIYWA |
| | STNYNPSLKSRVTISVDTSKNQFSLKLSS | STRESGVPDRFSGSGSGTDFTLTISSL |
| | VTAADTAVYYCARARGPWGFDPWGQG | QPEDSATYYCQQSYDIPYTFGQGTK |
| | TLVTVSS | LEIK |
| | (SEQ ID NO: 10) | (SEQ ID NO: 11) |
| ADI-28226 | QVQLQQWGAGLLKPSETLSLTCAVYGG | DIQMTQSPSTLSASVGDRVTITCRAS |
| (C26) | SFSGYYWSWIRQPPGKGLEWIGEIDHSG | QSISSWLAWYQQKPGKAPKLLIYKA |
| | STNYNPSLKSRVTISVDTSKNQFSLKLSS | SSLESGVPSRFSGSGSGTEFTLTISSL |
| | VTAADTAVYYCARARGPWSFDPWGQG | QPDDFATYYCQQYGSFPITFGGGTK |
| | TLVTVSS | VEIK |
| | (SEQ ID NO: 1) | (SEQ ID NO: 12) |
| ADI-28154 | QVQLQQWGAGLLKPSETLSLTCAVYGG | DIQMTQSPSTLSASVGDRVTITCRAS |
| | SFSGYYWSWIRQPPGKGLEWIGEIDHSG | QSISSWLAWYQQKPGKAPKLLIYKA |
| | STNYNPSLKSRVTISVDTSKNQFSLKLSS | SSLESGVPSRFSGSGSGTDFTLTISSL |
| | VTAADTAVYYCARARGPWSFDPWGQG | QPDDFATYYCQQSKEVPWTFGQGT |
| | TLVTVSS | KVEIK |
| | (SEQ ID NO: 1) | (SEQ ID NO: 13) |
| ADI-29399 | QVQLQQWGAGLLKPSETLSLTCAVYGG | DIQMTQSPSTLSASVGDRVTITCRAS |
| | SFSGYYWSWIRQPPGKGLEWIGEIDHSG | QSISSWLAWYQQKPGKAPKLLIYKA |
| | STNYNPSLKSRVTISVDTSKNQFSLKLSS | SSLESGVPSRFSGSGSGTEFTLTISSL |
| | VTAADTAVYYCARARGPWSFDPWGQG | QPDDFATYYCQQYNSFPTFGGGTKV |
| | TLVTVSS | EIK |
| | (SEQ ID NO: 1) | (SEQ ID NO: 14) |
| ADI-29401 | QVQLQQWGAGLLKPSETLSLTCAVYGG | DIQMTQSPSTLSASVGDRVTITCRAS |
| | SFSGYYWSWIRQPPGKGLEWIGEIDHSG | QSIGSWLAWYQQKPGKAPKLLIYKA |
| | STNYNPSLKSRVTISVDTSKNQFSLKLSS | SSLESGVPSRFSGSGSGTEFTLTISSL |
| | VTAADTAVYYCARARGPWSFDPWGQG | QPDDFATYYCQQYDIYPTFGGGTKV |
| | TLVTVSS | EIK |
| | (SEQ ID NO: 1) | (SEQ ID NO: 15) |
| ADI-29403 | QVQLQQWGAGLLKPSETLSLTCAVYGG | DIQMTQSPSTLSASVGDRVTITCRAS |
| | SFSGYYWSWIRQPPGKGLEWIGEIDHSG | QSISSWLAWYQQKPGKAPKLLIYKA |
| | STNYNPSLKSRVTISVDTSKNQFSLKLSS | SSLESGVPSRFSGSGSGTEFTLTISSL |
| | VTAADTAVYYCARARGPWSFDPWGQG | QPDDFATYYCQQYDSYPTFGGGTK |
| | TLVTVSS | VEIK |
| | (SEQ ID NO: 1) | (SEQ ID NO: 16) |
| ADI-29405 | QVQLQQWGAGLLKPSETLSLTCAVYGG | DIQMTQSPSTLSASVGDRVTITCRAS |
| | SFSGYYWSWIRQPPGKGLEWIGEIDHSG | QSISSWLAWYQQKPGKAPKLLIYKA |
| | STNYNPSLKSRVTISVDTSKNQFSLKLSS | SSLESGVPSRFSGSGSGTEFTLTISSL |
| | VTAADTAVYYCARARGPWSFDPWGQG | QPDDFATYYCQQYGSFPTFGGGTKV |
| | TLVTVSS | EIK |
| | (SEQ ID NO: 1) | (SEQ ID NO: 17) |
| ADI-29407 | QVQLQQWGAGLLKPSETLSLTCAVYGG | DIQMTQSPSTLSASVGDRVTITCRAS |
| | SFSGYYWSWIRQPPGKGLEWIGEIDHSG | QSISSWLAWYQQKPGKAPKLLIYKA |
| | STNYNPSLKSRVTISVDTSKNQFSLKLSS | SSLESGVPSRFSGSGSGTEFTLTISSL |
| | VTAADTAVYYCARARGPWSFDPWGQG | QPDDFATYYCQQYQSFPTFGGGTKV |
| | TLVTVSS | EIK |
| | (SEQ ID NO: 1) | (SEQ ID NO: 18) |
| ADI-29419 | QVQLQQWGAGLLKPSETLSLTCAVYGG | DIQMTQSPSTLSASVGDRVTITCRAS |
| | SFSGYYWSWIRQPPGKGLEWIGEIDHSG | QSISSWLAWYQQKPGKAPKLLIYKA |
| | STNYNPSLKSRVTISVDTSKNQFSLKLSS | SSLESGVPSRFSGSGSGTEFTLTISSL |
| | VTAADTAVYYCARARGPWSFDPWGQG | QPDDFATYYCQQYSSFSTFGGGTKV |
| | TLVTVSS | EIK |
| | (SEQ ID NO: 1) | (SEQ ID NO: 19) |
| ADI-29421 | QVQLQQWGAGLLKPSETLSLTCAVYGG | DIQMTQSPSTLSASVGDRVTITCRAS |
| | SFSGYYWSWIRQPPGKGLEWIGEIDHSG | QSISSWLAWYQQKPGKAPKLLIYKA |
| | STNYNPSLKSRVTISVDTSKNQFSLKLSS | SSLESGVPSRFSGSGSGTEFTLTISSL |
| | VTAADTAVYYCARARGPWSFDPWGQG | QPDDFATYYCQQYESYSTFGGGTKV |
| | TLVTVSS | EIK |
| | (SEQ ID NO: 1) | (SEQ ID NO: 20) |
| ADI-29424 | QVQLQQWGAGLLKPSETLSLTCAVYGG | DIQMTQSPSTLSASVGDRVTITCRAS |
| | SFSGYYWSWIRQPPGKGLEWIGEIDHSG | QSISSWLAWYQQKPGKAPKLLIYKA |
| | STNYNPSLKSRVTISVDTSKNQFSLKLSS | SSLESGVPSRFSGSGSGTEFTLTISSL |
| | VTAADTAVYYCARARGPWSFDPWGQG | QPDDFATYYCQQYDSFITFGGGTKV |
| | TLVTVSS | EIK |
| | (SEQ ID NO: 1) | (SEQ ID NO: 21) |
| ADI-29425 | QVQLQQWGAGLLKPSETLSLTCAVYGG | DIQMTQSPSTLSASVGDRVTITCRAS |
| | SFSGYYWSWIRQPPGKGLEWIGEIDHSG | QSISSWLAWYQQKPGKAPKLLIYKA |
| | STNYNPSLKSRVTISVDTSKNQFSLKLSS | SSLESGVPSRFSGSGSGTEFTLTISSL |
| | VTAADTAVYYCARARGPWSFDPWGQG | QPDDFATYYCQQYQSYPTFGGGTK |
| | TLVTVSS | VEIK |
| | (SEQ ID NO: 1) | (SEQ ID NO: 22) |
| ADI-29426 | QVQLQQWGAGLLKPSETLSLTCAVYGG | DIQMTQSPSTLSASVGDRVTITCRAS |
| | SFSGYYWSWIRQPPGKGLEWIGEIDHSG | QSIGSWLAWYQQKPGKAPKLLIYKA |
| | STNYNPSLKSRVTISVDTSKNQFSLKLSS | SSLESGVPSRFSGSGSGTEFTLTISSL |
| | VTAADTAVYYCARARGPWSFDPWGQG | QPDDFATYYCQQYHSFPTFGGGTKV |
| | TLVTVSS | EIK |
| | (SEQ ID NO: 1) | (SEQ ID NO: 23) |
| ADI-29429 | QVQLQQWGAGLLKPSETLSLTCAVYGG | DIQMTQSPSTLSASVGDRVTITCRAS |
| | SFSGYYWSWIRQPPGKGLEWIGEIDHSG | QSIGSWLAWYQQKPGKAPKLLIYKA |
| | STNYNPSLKSRVTISVDTSKNQFSLKLSS | SSLESGVPSRFSGSGSGTEFTLTISSL |
| | VTAADTAVYYCARARGPWSFDPWGQG | QPDDFATYYCQQYELYSYTFGGGTK |
| | TLVTVSS | VEIK |
| | (SEQ ID NO: 1) | (SEQ ID NO: 24) |
| ADI-29447 | QVQLQQWGAGLLKPSETLSLTCAVYGG | DIQMTQSPSTLSASVGDRVTITCRAS |
| (F47) | SFSGYYWSWIRQPPGKGLEWIGEIDHSG | QSISSWLAWYQQKPGKAPKLLIYKA |
| | STNYNPSLKSRVTISVDTSKNQFSLKLSS | SSLESGVPSRFSGSGSGTEFTLTISSL |
| | VTAADTAVYYCARARGPWSFDPWGQG | QPDDFATYYCQQYDTFITFGGGTKV |
| | TLVTVSS | EIK |
| | (SEQ ID NO: 1) | (SEQ ID NO: 25) |
| ADI-27727 | QVQLVQSGAEVKKPGSSVKVSCKASGG | DIVMTQSPDSLAVSLGERATINCKSS |
| | TFSSYAISWVRQAPGQGLEWMGGIIPIFG | QSVLYSSNNKNYLAWYQQKPGQPP |
| | TANYAQKFQGRVTITADESTSTAYMELS | KLLIYWASTRESGVPDRFSGSGSGTD |
| | SLRSEDTAVYYCARGDSSIRHAYYYYG | FTLTISSLQAEDVAVYYCQQYYSTPI |
| | MDVWGQGTTVTVSS | TFGGGTKVEIK |
| | (SEQ ID NO: 26) | (SEQ ID NO: 32) |
| | CDR1-GTFSSYAIS (non-Kabat) (SEQ ID | CDR1 (SEQ ID NO: 33)- |
| | NO: 27) or SYAIS (SEQ ID NO: 28) | KSSQSVLYSSNNKNYLA |
| | CDR2 (SEQ ID NO: 29)- | CDR2 (SEQ ID NO: 34) |
| | GIIPIFGTANYAQKFQG | WASTRES |
| | CDR3-ARGDSSIRHAYYYYGMDV | CDR3 (SEQ ID NO: 35)- |
| | (non-Kabat) (SEQ ID NO: 30) or | QQYYSTPIT |
| | GDSSIRHAYYYYGMDV (SEQ ID NO: 31) | |
| ADI-29443 | QLQLQESGPGLVKPSETLSLTCTVSGGSI | EIVLTQSPATLSLSPGERATLSCRASQ |
| (F43) | SSSSYYWGWIRQPPGKGLEWIGSIYYSGS | SVSRYLAWYQQKPGQAPRLLIYDAS |
| | TYYNPSLKSRVTISVDTSKNQFSLKLSSV | NRATGIPARFSGSGSGTDFTLTISSLE |
| | TAADTAVYYCARGSDRFHPYFDYWGQG | PEDFAVYYCQQFDTWPPTFGGGTKV |
| | TLVTVSS | EIK |
| | (SEQ ID NO: 36) | (SEQ ID NO: 42) |
| | CDR1-GSISSSSYYWG (non-Kabat) (SEQ | CDR1 (SEQ ID NO: 43)- |
| | ID NO: 37) or SSSYYWG (SEQ ID NO: 38) | RASQSVSRYLA |
| | CDR2 (SEQ ID NO: 39)- | CDR2 (SEQ ID NO: 44)- |
| | SIYYSGSTYYNPSLKS | DASNRAT |
| | CDR3-ARGSDRFHPYFDY (non-Kabat) | CDR3 (SEQ ID NO: 45)- |
| | (SEQ ID NO: 40) or GSDRFHPYFDY (SEQ | QQFDTWPPT |
| | ID NO: 41) | |
| ADI-29404 | QVQLQQWGAGLLKPSETLSLTCAVYGG | DIQMTQSPSTLSASVGDRVTITCRAS |
| (F04) | SFSGYYWSWIRQPPGKGLEWIGEIDHSG | QSISSWLAWYQQKPGKAPKLLIYKA |
| | STNYNPSLKSRVTISVDTSKNQFSLKLSS | SSLESGVPSRFSGSGSGTEFTLTISSL |
| | VTAADTAVYYCARARGPWSFDPWGQG | QPDDFATYYCEQYDSYPTFGGGTKV |
| | TLVTVSS (SEQ ID NO: 1) | EIK |
| | | (SEQ ID NO: 46) |
| ADI-28200 | QVQLVQSGAEVKKPGSSVKVSCKASGG | DIVMTQSPDSLAVSLGERATINCESS |
| | TFSSYAISWVRQAPGQGLEWMGGIIPIFG | QSLLNSGNQKNYLTWYQQKPGQPP |
| | TANYAQKFQGRVTITADESTSTAYMELS | KPLIYWASTRESGVPDRFSGSGSGTD |
| | SLRSEDTAVYYCARRGRKASGSFYYYY | FTLTISSLQAEDVAVYYCQNDYSYP |
| | GMDVWGQGTTVTVSS | YTFGQGTKLEIK |
| | (SEQ ID NO: 47) | (SEQ ID NO: 49) |
| | CDR1 (SEQ ID NO: 27)-GTFSSYAIS | CDR1 (SEQ ID NO: 50)- |
| | CDR2 (SEQ ID NO: 29)- | ESSQSLLNSGNQKNYLT |
| | GIIPIFGTANYAQKFQG | CDR2 (SEQ ID NO: 34)-WASTRES |
| | CDR3 (SEQ ID NO: 48)- | CDR3 (SEQ ID NO: 51)-QNDYSYPYT |
| | ARRGRKASGSFYYYYGMDV | |
| ADI-29379 | QVQLVQSGAEVKKPGASVKVSCKASGY | EIVMTQSPATLSVSPGERATLSCRAS |
| (E79) | TFTSYYMHWVRQAPGQGLEWMGIINPS | QSVSSNLAWYQQKPGQAPRLLIYGA |
| | GGSTSYAQKFQGRVTMTRDTSTSTVYM | STRATGIPARFSGSGSGTEFTLTISSL |
| | ELSSLRSEDTAVYYCARGAPNYGDTTHD | QSEDFAVYYCQQYDDWPFTFGGGT |
| | YYYMDVWGKGTTVTVSS | KVEIK |
| | (SEQ ID NO: 52) | (SEQ ID NO: 58) |
| | CDR1 (SEQ ID NO: 53)-YTFTSYYMH | CDR1 (SEQ ID NO: 59)- |
| | (non-Kabat) or SYYMH (SEQ ID NO: 54) | RASQSVSSNLA |
| | CDR2 (SEQ ID NO: 55)- | CDR2 (SEQ ID NO: 60)-GASTRAT |
| | IINPSGGSTSYAQKFQG | CDR3 (SEQ ID NO: 61)-QQYDDWPFT |
| | CDR3-ARGAPNYGDTTHDYYYMDV | |
| | (non-Kabat) (SEQ ID NO: 56) or | |
| | GAPNYGDTTHDYYYMDV (SEQ ID | |
| | NO: 57) | |
| ADI-29463 | QVQLVQSGAEVKKPGASVKVSCKASGY | EIVLTQSPGTLSLSPGERATLSCRASQ |
| (F63) | TFTGYYMHWVRQAPGQGLEWMGWINP | SVSSNLAWYQQKPGQAPRLLIYGAS |
| | NSGGTNYAQKFQGRVTMTRDTSISTAY | TRATGIPARFSGSGSGTEFTLTISSLQ |
| | MELSRLRSDDTAVYYCARDTGEYYDTD | SEDFAVYYCQQDDYWPPTFGGGTK |
| | DHGMDVWGQGTTVTVSS | VEIK |
| | (SEQ ID NO: 62) | (SEQ ID NO: 68) |
| | CDR1-YTFTGYYMH (non-Kabat) (SEQ ID | CDR1 (SEQ ID NO: 59)- |
| | NO: 63) or GYYMH (SEQ ID NO: 64) | RASQSVSSNLA |
| | CDR2 (SEQ ID NO: 65)- | CDR2 (SEQ ID NO: 60)-GASTRAT |
| | WINPNSGGTNYAQKFQG | CDR3 (SEQ ID NO: 69)-QQDDYWPPT |
| | CDR3-ARDTGEYYDTDDHGMDV (non- | |
| | Kabat) (SEQ ID NO: 66) or | |
| | DTGEYYDTDDHGMDV (SEQ ID NO: 67) | |
| ADI-27744 | EVQLLESGGGLVQPGGSLRLSCAASGFT | DIQMTQSPSSVSASVGDRVTITCRAS |
| (A49) | FSSYAMSWVRQAPGKGLEWVSAISGSG | QGIDSWLAWYQQKPGKAPKLLIYA |
| | GSTYYADSVKGRFTISRDNSKNTLYLQM | ASSLQSGVPSRFSGSGSGTDFTLTISS |
| | NSLRAEDTAVYYCAKDGGYYDSGAGD | LQPEDFATYYCQQGVSYPRTFGGGT |
| | YWGQGTLVTVSS | KVEIK |
| | (SEQ ID NO: 70) | (SEQ ID NO: 75) |
| | CDR1-FTFSSYAMS (non-Kabat) (SEQ ID | CDR1 (SEQ ID NO: 76)- |
| | NO: 71) or SYAMS (SEQ ID NO: 115) | RASQGIDSWLA |
| | CDR2 (SEQ ID NO: 72)- | CDR2 (SEQ ID NO: 77)-AASSLQS |
| | AISGSGGSTYYADSVKG | CDR3 (SEQ ID NO: 78)-QQGVSYPRT |
| | CDR3-AKDGGYYDSGAGDY (non-Kabat) | |
| | (SEQ ID NO: 73) or DGGYYDSGAGDY | |
| | (SEQ ID NO: 74) | |
| ADI-27749 | EVQLVESGGGLVKPGGSLRLSCAASGFT | DIQMTQSPSSVSASVGDRVTITCRAS |
| (A44) | FSSYSMNWVRQAPGKGLEWVSSISSSSS | QGISSWLAWYQQKPGKAPKLLIYAA |
| | YIYYADSVKGRFTISRDNAKNSLYLQMN | SSLQSGVPSRFSGSGSGTDFTLTISSL |
| | SLRAEDTAVYYCARGAPMGAAAGWFD | QPEDFATYYCQQGVSFPRTFGGGTK |
| | PWGQGTLVTVSS | VEIK |
| | (SEQ ID NO: 79) | (SEQ ID NO: 85) |
| | CDR1-FTFSSYSMN (SEQ ID NO: 80) (non- | CDR1 (SEQ ID NO: 86)- |
| | Kabat) or SYSMN (SEQ ID NO: 81) or | RASQGISSWLA |
| | GFTFSSY (SEQ ID NO: 173) (Chotia) | CDR2 (SEQ ID NO: 77)-AASSLQS |
| | CDR2 (SEQ ID NO: 82)- | CDR3 (SEQ ID NO: 87)-QQGVSFPRT |
| | SISSSSSYIYYADSVKG or SSSSSY (SEQ | (Kabat and Chotia) |
| | ID NO: 174 (Chotia) | |
| | CDR3-ARGAPMGAAAGWFDP (SEQ ID | |
| | NO: 83) (non-Kabat) or | |
| | GAPMGAAAGWFDP (SEQ ID NO: 84) | |
| | (Kabat and Chotia) | |
| | scFv (VL-VH) with Q44C in VH and G100C in VL, linker italicized: | |
| | DIQMTQSPSSVSASVGDRVTITCRASQGISSWLAWYQQKPGKAPKLLIYAASSLQS | |
| | GVPSRFSGSGSGTDFTLTISSLQPEDFATYYCQQGVSFPRTFGCGTKVEIKGGGGSG | |
| | GGGSGGGGSGGGGSEVQLVESGGGLVKPGGSLRLSCAASGFTFSSYSMNWVRQA | |
| | PGKCLEWVSSISSSSSYIYYADSVKGRFTISRDNAKNSLYLQMNSLRAEDTAVYYC | |
| | ARGAPMGAAAGWFDPWGQGTLVTVSS (SEQ ID NO: 88) | |
| ADI-29378 | QVQLVQSGAEVKKPGASVKVSCKASGY | EIVLTQSPATLSLSPGERATLSCRASQ |
| (E78) | TFTSYYMHWVRQAPGQGLEWMGIINPS | SVSSYLAWYQQKPGQAPRLLIYDAS |
| | GGSTSYAQKFQGRVTMTRDTSTSTVYM | NRATGIPARFSGSGSGTDFTLTISSLE |
| | ELSSLRSEDTAVYYCAREGAGFAYGMD | PEDFAVYYCQQSDNWPFTFGGGTK |
| | YYYMDVWGKGTTVTVSS | VEIK |
| | (SEQ ID NO: 89) | (SEQ ID NO: 92) |
| | CDR1-YTFTSYYMH (SEQ ID NO: 53) | CDR1 (SEQ ID NO: 93)- |
| | (non-Kabat) or SYYMH (SEQ ID NO: 54) | RASQSVSSYLA |
| | CDR2 (SEQ ID NO: 55)- | CDR2 (SEQ ID NO: 44)-DASNRAT |
| | IINPSGGSTSYAQKFQG | CDR3 (SEQ ID NO: 94)-QQSDNWPFT |
| | CDR3-AREGAGFAYGMDYYYMDV | |
| | (SEQ ID NO: 90) (non-Kabat) or | |
| | EGAGFAYGMDYYYMDV (SEQ ID NO: 91) | |
| A49MI | EVQLVESGGGLVKPGGSLRLSCAASGFT | DIQMTQSPSSVSASVGDRVTITCRAS |
| | FSSYSMNWVRQAPGKGLEWVSSISSSSS | QGISSWLAWYQQKPGKAPKLLIYAA |
| | YIYYADSVKGRFTISRDNAKNSLYLQMN | SSLQSGVPSRFSGSGSGTDFTLTISSL |
| | SLRAEDTAVYYCARGAPIGAAAGWFDP | QPEDFATYYCQQGVSFPRTFGGGTK |
| | WGQGTLVTVSS (SEQ ID NO: 95) | VEIK |
| | CDR1: FTFSSYSMN (SEQ ID NO: 80) (non- | (SEQ ID NO: 85) |
| | Kabat) or SYSMN (SEQ ID NO: 81) | CDR1 (SEQ ID NO: 86)- |
| | CDR2: SISSSSSYIYYADSVKG | RASQGISSWLA |
| | (SEQ ID NO: 82) | CDR2 (SEQ ID NO: 77)-AASSLQS |
| | CDR3: ARGAPIGAAAGWFDP (SEQ ID | CDR3 (SEQ ID NO: 87)-QQGVSFPRT |
| | NO: 96) (non-Kabat) or GAPIGAAAGWFDP | |
| | (SEQ ID NO: 97) | |
| A49MQ | EVQLVESGGGLVKPGGSLRLSCAASGFT | DIQMTQSPSSVSASVGDRVTITCRAS |
| | FSSYSMNWVRQAPGKGLEWVSSISSSSS | QGISSWLAWYQQKPGKAPKLLIYAA |
| | YIYYADSVKGRFTISRDNAKNSLYLQMN | SSLQSGVPSRFSGSGSGTDFTLTISSL |
| | SLRAEDTAVYYCARGAPQGAAAGWFDP | QPEDFATYYCQQGVSFPRTFGGGTK |
| | WGQGTLVTVSS | VEIK |
| | (SEQ ID NO: 98) | (SEQ ID NO: 85) |
| | CDR1: FTFSSYSMN (SEQ ID NO: 80) (non- | CDR1 (SEQ ID NO: 86)- |
| | Kabat) or SYSMN (SEQ ID NO: 81) | RASQGISSWLA |
| | CDR2: SISSSSSYIYYADSVKG | CDR2 (SEQ ID NO: 77)-AASSLOS |
| | (SEQ ID NO: 82) | CDR3 (SEQ ID NO: 87)-QQGVSFPRT |
| | CDR3-ARGAPQGAAAGWFDP (SEQ ID | |
| | NO: 99) (non-Kabat) or GAPQGAAAGWFDP | |
| | (SEQ ID NO: 100) | |
| A49ML | EVQLVESGGGLVKPGGSLRLSCAASGFT | DIQMTQSPSSVSASVGDRVTITCRAS |
| | FSSYSMNWVRQAPGKGLEWVSSISSSSS | QGISSWLAWYQQKPGKAPKLLIYAA |
| | YIYYADSVKGRFTISRDNAKNSLYLQMN | SSLQSGVPSRFSGSGSGTDFTLTISSL |
| | SLRAEDTAVYYCARGAPLGAAAGWFDP | QPEDFATYYCQQGVSFPRTFGGGTK |
| | WGQGTLVTVSS | VEIK |
| | (SEQ ID NO: 101) | (SEQ ID NO: 85) |
| | CDR1: FTFSSYSMN (SEQ ID NO: 80) (non- | CDR1 (SEQ ID NO: 86)- |
| | Kabat) or SYSMN (SEQ ID NO: 81) | RASQGISSWLA |
| | CDR2: SISSSSSYIYYADSVKG | CDR2 (SEQ ID NO: 77)-AASSLQS |
| | (SEQ ID NO: 82) | CDR3 (SEQ ID NO: 87)-QQGVSFPRT |
| | CDR3-ARGAPLGAAAGWFDP (SEQ ID | |
| | NO: 102) (non-Kabat) or | |
| | GAPLGAAAGWFDP (SEQ ID NO: 103) | |
| A49MF | EVQLVESGGGLVKPGGSLRLSCAASGFT | DIQMTQSPSSVSASVGDRVTITCRAS |
| | FSSYSMNWVRQAPGKGLEWVSSISSSSS | QGISSWLAWYQQKPGKAPKLLIYAA |
| | YIYYADSVKGRFTISRDNAKNSLYLQMN | SSLQSGVPSRFSGSGSGTDFTLTISSL |
| | SLRAEDTAVYYCARGAPFGAAAGWFDP | QPEDFATYYCQQGVSFPRTFGGGTK |
| | WGQGTLVTVSS | VEIK |
| | (SEQ ID NO: 104) | (SEQ ID NO: 85) |
| | CDR1: FTFSSYSMN (SEQ ID NO: 80) (non- | CDR1 (SEQ ID NO: 86)- |
| | Kabat) or SYSMN (SEQ ID NO: 81) | RASQGISSWLA |
| | CDR2: SISSSSSYIYYADSVKG | CDR2 (SEQ ID NO: 77)-AASSLQS |
| | (SEQ ID NO: 82) | CDR3 (SEQ ID NO: 87)-QQGVSFPRT |
| | CDR3-ARGAPFGAAAGWFDP (SEQ ID | |
| | NO: 105) (non-Kabat) or | |
| | GAPFGAAAGWFDP (SEQ ID NO: 106) | |
| A49MV | EVQLVESGGGLVKPGGSLRLSCAASGFT | DIQMTQSPSSVSASVGDRVTITCRAS |
| | FSSYSMNWVRQAPGKGLEWVSSISSSSS | QGISSWLAWYQQKPGKAPKLLIYAA |
| | YIYYADSVKGRFTISRDNAKNSLYLQMN | SSLQSGVPSRFSGSGSGTDFTLTISSL |
| | SLRAEDTAVYYCARGAPVGAAAGWFDP | QPEDFATYYCQQGVSFPRTFGGGTK |
| | WGQGTLVTVSS | VEIK |
| | (SEQ ID NO: 107) | (SEQ ID NO: 85) |
| | CDR1: FTFSSYSMN (SEQ ID NO: 80) (non- | CDR1 (SEQ ID NO: 86)- |
| | Kabat) or SYSMN (SEQ ID NO: 81) | RASQGISSWLA |
| | CDR2: SISSSSSYIYYADSVKG | CDR2 (SEQ ID NO: 77)-AASSLQS |
| | (SEQ ID NO: 82) | CDR3 (SEQ ID NO: 87)-QQGVSFPRT |
| | CDR3-ARGAPVGAAAGWFDP (SEQ ID | |
| | NO: 108) (non-Kabat) or | |
| | GAPVGAAAGWFDP (SEQ ID NO: 109) | |
| A49- | EVQLVESGGGLVKPGGSLRLSCAASGFT | DIQMTQSPSSVSASVGDRVTITCRAS |
| consensus | FSSYSMNWVRQAPGKGLEWVSSISSSSS | QGISSWLAWYQQKPGKAPKLLIYAA |
| | YIYYADSVKGRFTISRDNAKNSLYLQMN | SSLQSGVPSRFSGSGSGTDFTLTISSL |
| | SLRAEDTAVYYCARGAPXGAAAGWFDP | QPEDFATYYCQQGVSFPRTFGGGTK |
| | WGQGTLVTVSS, wherein X is M, L, I, V, | VEIK |
| | Q, or F | (SEQ ID NO: 85) |
| | (SEQ ID NO: 110) | CDR1 (SEQ ID NO: 86)- |
| | CDR1: FTFSSYSMN (SEQ ID NO: 80) (non- | RASQGISSWLA |
| | Kabat) or SYSMN (SEQ ID NO: 81) | CDR2 (SEQ ID NO: 77)-AASSLQS |
| | CDR2: SISSSSSYIYYADSVKG | CDR3 (SEQ ID NO: 87)-QQGVSFPRT |
| | (SEQ ID NO: 82) | |
| | CDR3 ARGAPXGAAAGWFDP (SEQ ID | |
| | NO: 111) (non-Kabat) or | |
| | GAPXGAAAGWFDP (SEQ ID NO: 112), | |
| | wherein X is M, L, I, V, Q, or F | |
| NKG2D | QVQLVESGGGLVKPGGSLRLSCAASGFT | QSALTQPASVSGSPGQSITISCSGSSS |
| binder in US | FSSYGMHWVRQAPGKGLEWVAFIRYDG | NIGNNAVNWYQQLPGKAPKLLIYY |
| 9,273,136 | SNKYYADSVKGRFTISRDNSKNTLYLQM | DDLLPSGVSDRFSGSKSGTSAFLAIS |
| | NSLRAEDTAVYYCAKDRGLGDGTYFDY | GLQSEDEADYYCAAWDDSLNGPVF |
| | WGQGTTVTVSS (SEQ ID NO: 113) | GGGTKLTVL (SEQ ID NO: 114) |
| NKG2D | QVHLQESGPGLVKPSETLSLTCTVSDDSI | EIVLTQSPGTLSLSPGERATLSCRASQ |
| binder in US | SSYYWSWIRQPPGKGLEWIGHISYSGSA | SVSSSYLAWYQQKPGQAPRLLIYGA |
| 7,879,985 | NYNPSLKSRVTISVDTSKNQFSLKLSSVT | SSRATGIPDRFSGSGSGTDFTLTISRL |
| | AADTAVYYCANWDDAFNIWGQGTMVT | EPEDFAVYYCQQYGSSPWTFGQGTK |
| | VSS (SEQ ID NO: 116) | VEIK (SEQ ID NO: 117) |

In certain embodiments, the first antigen-binding site that binds NKG2D (e.g., human NKG2D) includes a VH that has an amino acid sequence at least 90% (e.g., at least 91%, at least 92%, at least 93%, at least 94%, at least 95%, at least 96%, at least 97%, at least 98%, at least 99%, or 100%) identical to the VH of an antibody disclosed in Table 1, and a VL that has an amino acid sequence at least 90% (e.g., at least 91%, at least 92%, at least 93%, at least 94%, at least 95%, at least 96%, at least 97%, at least 98%, at least 99%, or 100%) identical to the VL of the same antibody disclosed in Table 1. In certain embodiments, the first antigen-binding site includes the heavy chain CDR1, CDR2, and CDR3 and the light chain CDR1, CDR2, and CDR3, determined under Kabat (see Kabat et al., (1991) Sequences of Proteins of Immunological Interest, NIH Publication No. 91-3242, Bethesda), Chothia (see, e.g., Chothia C & Lesk A M, (1987), J. Mol. Biol. 196: 901-917), MacCallum (see MacCallum R M et al., (1996) J. Mol. Biol. 262: 732-745), or any other CDR determination method known in the art, of the VH and VL sequences of an antibody disclosed in Table 1. In certain embodiments, the first antigen-binding site includes the heavy chain CDR1, CDR2, and CDR3 sequences and the light chain CDR1, CDR2, and CDR3 sequences of an antibody disclosed in Table 1.

In certain embodiments, the first antigen-binding site that binds NKG2D (e.g., human NKG2D) includes a Fab including a VH that has an amino acid sequence at least 90% (e.g., at least 91%, at least 92%, at least 93%, at least 94%, at least 95%, at least 96%, at least 97%, at least 98%, at least 99%, or 100%) identical to the VH of an antibody disclosed in Table 1, and a VL that has an amino acid sequence at least 90% (e.g., at least 91%, at least 92%, at least 93%, at least 94%, at least 95%, at least 96%, at least 97%, at least 98%, at least 99%, or 100%) identical to the VL of the same antibody disclosed in Table 1. In certain embodiments, the first antigen-binding site includes a Fab with the heavy chain CDR1, CDR2, and CDR3 and the light chain CDR1, CDR2, and CDR3, determined under Kabat (see Kabat et al., (1991) Sequences of Proteins of Immunological Interest, NIH Publication No. 91-3242, Bethesda), Chothia (see, e.g., Chothia C & Lesk A M, (1987), J. Mol. Biol. 196: 901-917), MacCallum (see MacCallum R M et al., (1996) J. Mol. Biol. 262: 732-745), or any other CDR determination method known in the art, of the VH and VL sequences of an antibody disclosed in Table 1. In certain embodiments, the first antigen-binding site includes a Fab with the heavy chain CDR1, CDR2, and CDR3 sequences and the light chain CDR1, CDR2, and CDR3 sequences of an antibody disclosed in Table 1.

In certain embodiments, the first antigen-binding site that binds to NKG2D includes a heavy chain variable domain derived from SEQ ID NO:1, such as by having an amino acid sequence at least 90% (e.g., at least 90%, at least 91%, at least 92%, at least 93%, at least 94%, at least 95%, at least 96%, at least 97%, at least 98%, at least 99%, or 100%) identical to SEQ ID NO:1, and/or incorporating amino acid sequences identical to the CDR1 (SEQ ID NO:2), CDR2 (SEQ ID NO:3), and CDR3 (SEQ ID NO:4) sequences of SEQ ID NO:1. The heavy chain variable domain related to SEQ ID NO:1 can be coupled with a variety of light chain variable domains to form an NKG2D binding site. For example, the first antigen-binding site that incorporates a heavy chain variable domain related to SEQ ID NO:1 can further incorporate a light chain variable domain selected from the sequences derived from SEQ ID NOs: 5, 6, 7, 8, 9, 12, 13, 14, 15, 16, 17, 18, 19, 20, 21, 22, 23, 24, 25, and 46. For example, the first antigen-binding site can incorporate a heavy chain variable domain with amino acid sequences at least 90% (e.g., at least 90%, at least 91%, at least 92%, at least 93%, at least 94%, at least 95%, at least 96%, at least 97%, at least 98%, at least 99%, or 100%) identical to SEQ ID NO:1 and a light chain variable domain with amino acid sequences at least 90% (e.g., at least 90%, at least 91%, at least 92%, at least 93%, at least 94%, at least 95%, at least 96%, at least 97%, at least 98%, at least 99%, or 100%) identical to any one of the sequences selected from SEQ ID NOs: 5, 6, 7, 8, 9, 12, 13, 14, 15, 16, 17, 18, 19, 20, 21, 22, 23, 24, 25, and 46.

In certain embodiments, the first antigen-binding site that binds NKG2D includes a VH with an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:26, and a VL that with an amino acid sequence at least 90% (e.g., at least 91%, at least 92%, at least 93%, at least 94%, at least 95%, at least 96%, at least 97%, at least 98%, at least 99%, or 100%) identical to SEQ ID NO:32. In certain embodiments, the VH includes CDR1, CDR2, and CDR3 with the amino acid sequences of SEQ ID NOs: 27 or 28, 29, and 30 or 31, respectively (e.g., SEQ ID NOs: 27, 29, and 30, respectively, or SEQ ID NOs: 28, 29, and 31, respectively). In certain embodiments, the VL includes CDR1, CDR2, and CDR3 with the amino acid sequences of SEQ ID NOs: 33, 34, and 35, respectively. In certain embodiments, the first antigen-binding site has (a) a VH with CDR1, CDR2, and CDR3 having the amino acid sequences of SEQ ID NOs: 27 or 28, 29, and 30 or 31, respectively (e.g., SEQ ID NOs: 27, 29, and 30, respectively, or SEQ ID NOs: 28, 29, and 31, respectively); and (b) a VL with CDR1, CDR2, and CDR3 having the amino acid sequences of SEQ ID NOs: 33, 34, and 35, respectively.

In certain embodiments, the first antigen-binding site that binds NKG2D includes a VH with an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:36, and a VL with an amino acid sequence at least 90% (e.g., at least 91%, at least 92%, at least 93%, at least 94%, at least 95%, at least 96%, at least 97%, at least 98%, at least 99%, or 100%) identical to SEQ ID NO:42. In certain embodiments, the VH has CDR1, CDR2, and CDR3 with the amino acid sequences of SEQ ID NOs: 37 or 38, 39, and 40 or 41, respectively (e.g., SEQ ID NOs: 37, 39, and 40, respectively, or SEQ ID NOs: 38, 39, and 41, respectively). In certain embodiments, the VL has CDR1, CDR2, and CDR3 with the amino acid sequences of SEQ ID NOs: 43, 44, and 45, respectively. In certain embodiments, the first antigen-binding site has (a) a VH with CDR1, CDR2, and CDR3 having the amino acid sequences of SEQ ID NOs: 37 or 38, 39, and 40 or 41, respectively (e.g., SEQ ID NOs: 37, 39, and 40, respectively, or SEQ ID NOs: 38, 39, and 41, respectively); and (b) a VL with CDR1, CDR2, and CDR3 having the amino acid sequences of SEQ ID NOs: 43, 44, and 45, respectively.

In certain embodiments, the first antigen-binding site that binds NKG2D includes a VH that has an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:47, and a VL that has an amino acid sequence at least 90% (e.g., at least 91%, at least 92%, at least 93%, at least 94%, at least 95%, at least 96%, at least 97%, at least 98%, at least 99%, or 100%) identical to SEQ ID NO:49. In certain embodiments, the VH includes CDR1, CDR2, and CDR3 having the amino acid sequences of SEQ ID NOs: 27, 29, and 48, respectively. In certain embodiments, the VL includes CDR1, CDR2, and CDR3 having the amino acid sequences of SEQ ID NOs: 50, 34, and 51, respectively. In certain embodiments, the first antigen-binding site includes (a) a VH that has CDR1, CDR2, and CDR3 with the amino acid sequences of SEQ ID NOs: 27, 29, and 48, respectively; and (b) a VL that has CDR1, CDR2, and CDR3 with the amino acid sequences of SEQ ID NOs: 50, 34, and 51, respectively.

In certain embodiments, the first antigen-binding site that binds NKG2D includes a VH that has an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:52, and a VL that has an amino acid sequence at least 90% (e.g., at least 91%, at least 92%, at least 93%, at least 94%, at least 95%, at least 96%, at least 97%, at least 98%, at least 99%, or 100%) identical to SEQ ID NO:58. In certain embodiments, the VH includes CDR1, CDR2, and CDR3 with the amino acid sequences of SEQ ID NOs: 53 or 54, 55, and 56 or 57, respectively (e.g., SEQ ID NOs: 53, 55, and 56, respectively, or SEQ ID NOs: 54, 55, and 57, respectively). In certain embodiments, the VL includes CDR1, CDR2, and CDR3 with the amino acid sequences of SEQ ID NOs: 59, 60, and 61, respectively. In certain embodiments, the first antigen-binding site includes (a) a VH with CDR1, CDR2, and CDR3 having the amino acid sequences of SEQ ID NOs: 53 or 54, 55, and 56 or 57, respectively (e.g., SEQ ID NOs: 53, 55, and 56, respectively, or SEQ ID NOs: 54, 55, and 57, respectively); and (b) a VL with CDR1, CDR2, and CDR3 having the amino acid sequences of SEQ ID NOs: 59, 60, and 61, respectively.

In certain embodiments, the first antigen-binding site that binds NKG2D includes a VH that has an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:62, and a VL that has an amino acid sequence at least 90% (e.g., at least 91%, at least 92%, at least 93%, at least 94%, at least 95%, at least 96%, at least 97%, at least 98%, at least 99%, or 100%) identical to SEQ ID NO:68. In certain embodiments, the VH includes CDR1, CDR2, and CDR3 with the amino acid sequences of SEQ ID NOs: 63 or 64, 65, and 66 or 67, respectively (e.g., SEQ ID NOs: 63, 65, and 66, respectively, or SEQ ID NOs: 64, 65, and 67, respectively). In certain embodiments, the VL includes CDR1, CDR2, and CDR3 with the amino acid sequences of SEQ ID NOs: 59, 60, and 69, respectively. In certain embodiments, the first antigen-binding site includes (a) a VH with CDR1, CDR2, and CDR3 having the amino acid sequences of SEQ ID NOs: 63 or 64, 65, and 66 or 67, respectively (e.g., SEQ ID NOs: 63, 65, and 66, respectively, or SEQ ID NOs: 64, 65, and 67, respectively); and (b) a VL with CDR1, CDR2, and CDR3 having the amino acid sequences of SEQ ID NOs: 59, 60, and 69, respectively.

In certain embodiments, the first antigen-binding site that binds NKG2D has a VH with an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:89, and a VL with an amino acid sequence at least 90% (e.g., at least 91%, at least 92%, at least 93%, at least 94%, at least 95%, at least 96%, at least 97%, at least 98%, at least 99%, or 100%) identical to SEQ ID NO:92. In certain embodiments, the VH has CDR1, CDR2, and CDR3 with the amino acid sequences of SEQ ID NOs: 53 or 54, 55, and 90 or 91, respectively (e.g., SEQ ID NOs: 53, 55, and 90, respectively, or SEQ ID NOs: 54, 55, and 91, respectively). In certain embodiments, the VL has CDR1, CDR2, and CDR3 with the amino acid sequences of SEQ ID NOs: 93, 44, and 94, respectively. In certain embodiments, the first antigen-binding site includes (a) a VH with CDR1, CDR2, and CDR3 having the amino acid sequences of SEQ ID NOs: 53 or 54, 55, and 90 or 91, respectively (e.g., SEQ ID NOs: 53, 55, and 90, respectively, or SEQ ID NOs: 54, 55, and 91, respectively); and (b) a VL with CDR1, CDR2, and CDR3 having the amino acid sequences of SEQ ID NOs: 93, 44, and 94, respectively.

In certain embodiments, the first antigen-binding site that binds NKG2D includes a VH with an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:70, and a VL with an amino acid sequence at least 90% (e.g., at least 91%, at least 92%, at least 93%, at least 94%, at least 95%, at least 96%, at least 97%, at least 98%, at least 99%, or 100%) identical to SEQ ID NO:75. In certain embodiments, the VH includes CDR1, CDR2, and CDR3 with the amino acid sequences of SEQ ID NOs: 71 or 115, 72, and 73 or 74, respectively (e.g., SEQ ID NOs: 71, 72, and 73, respectively, or SEQ ID NOs: 115, 72, and 74, respectively). In certain embodiments, the VL includes CDR1, CDR2, and CDR3 with the amino acid sequences of SEQ ID NOs: 76, 77, and 78, respectively. In certain embodiments, the first antigen-binding site includes (a) a VH with CDR1, CDR2, and CDR3 having the amino acid sequences of SEQ ID NOs: 71 or 115, 72, and 73 or 74, respectively (e.g., SEQ ID NOs: 71, 72, and 73, respectively, or SEQ ID NOs: 115, 72, and 74, respectively); and (b) a VL with CDR1, CDR2, and CDR3 having the amino acid sequences of SEQ ID NOs: 76, 77, and 78, respectively.

In certain embodiments, the first antigen-binding site that binds NKG2D includes a VH with an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:79, and a VL with an amino acid sequence at least 90% (e.g., at least 91%, at least 92%, at least 93%, at least 94%, at least 95%, at least 96%, at least 97%, at least 98%, at least 99%, or 100%) identical to SEQ ID NO:85. In certain embodiments, the VH includes CDR1, CDR2, and CDR3 with the amino acid sequences of SEQ ID NOs: 80 or 81, 82, and 83 or 84, respectively (e.g., SEQ ID NOs: 80, 82, and 83, respectively, or SEQ ID NOs: 81, 82, and 84, respectively). In certain embodiments, the VL includes CDR1, CDR2, and CDR3 with the amino acid sequences of SEQ ID NOs: 86, 77, and 87, respectively. In certain embodiments, the first antigen-binding site includes (a) a VH with CDR1, CDR2, and CDR3 having the amino acid sequences of SEQ ID NOs: 80 or 81, 82, and 83 or 84 respectively (e.g., SEQ ID NOs: 80, 82, and 83, respectively, or SEQ ID NOs: 81, 82, and 84, respectively); and (b) a VL with CDR1, CDR2, and CDR3 having the amino acid sequences of SEQ ID NOs: 86, 77, and 87, respectively.

In certain embodiments, the first antigen-binding site that binds NKG2D includes a VH with an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:95, and a VL with an amino acid sequence at least 90% (e.g., at least 91%, at least 92%, at least 93%, at least 94%, at least 95%, at least 96%, at least 97%, at least 98%, at least 99%, or 100%) identical to SEQ ID NO:85. In certain embodiments, the first antigen-binding site includes a VH having a sequence of SEQ ID NO:95, and a VL having a sequence of SEQ ID NO:85. In certain embodiments, the VH includes CDR1, CDR2, and CDR3 having the amino acid sequences of SEQ ID NOs: 80 or 81, 82, and 96 or 97, respectively (e.g., SEQ ID NOs: 80, 82, and 96, respectively, or SEQ ID NOs: 81, 82, and 97, respectively). In certain embodiments, the VL includes CDR1, CDR2, and CDR3 having the amino acid sequences of SEQ ID NOs: 86, 77, and 87, respectively. In certain embodiments, the first antigen-binding site includes (a) a VH that includes CDR1, CDR2, and CDR3 having the amino acid sequences of SEQ ID NOs: 80 or 81, 82, and 96 or 97, respectively (e.g., SEQ ID NOs: 80, 82, and 96, respectively, or SEQ ID NOs: 81, 82, and 97, respectively); and (b) a VL that includes CDR1, CDR2, and CDR3 having the amino acid sequences of SEQ ID NOs: 86, 77, and 87, respectively. In certain embodiments, the first antigen-binding site includes a VH having CDR1, CDR2, and CDR3 sequences of SEQ ID NOs: 80, 82, and 96, respectively, and a VL having CDR1, CDR2, and CDR3 sequences of SEQ ID NOs: 86, 77, and 87, respectively. In certain embodiments, the first antigen-binding site includes a VH having CDR1, CDR2, and CDR3 sequences of SEQ ID NOs: 81, 82, and 97, respectively, and a VL having CDR1, CDR2, and CDR3 sequences of SEQ ID NOs: 86, 77, and 87, respectively.

In certain embodiments, the first antigen-binding site is a Fab that binds NKG2D. The Fab includes a VH with an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:95, and a VL with an amino acid sequence at least 90% (e.g., at least 91%, at least 92%, at least 93%, at least 94%, at least 95%, at least 96%, at least 97%, at least 98%, at least 99%, or 100%) identical to SEQ ID NO:85. In certain embodiments, the Fab includes a VH with a sequence of SEQ ID NO:95, and a VL with a sequence of SEQ ID NO:85. In certain embodiments, the VH of the Fab includes CDR1, CDR2, and CDR3 with the amino acid sequences of SEQ ID NOs: 80 or 81, 82, and 96 or 97, respectively (e.g., SEQ ID NOs: 80, 82, and 96, respectively, or SEQ ID NOs: 81, 82, and 97, respectively). In certain embodiments, the VL of the Fab includes CDR1, CDR2, and CDR3 with the amino acid sequences of SEQ ID NOs: 86, 77, and 87, respectively. In certain embodiments, the Fab includes (a) a VH with CDR1, CDR2, and CDR3 having the amino acid sequences of SEQ ID NOs: 80 or 81, 82, and 96 or 97, respectively (e.g., SEQ ID NOs: 80, 82, and 96, respectively, or SEQ ID NOs: 81, 82, and 97, respectively); and (b) a VL with CDR1, CDR2, and CDR3 having the amino acid sequences of SEQ ID NOs: 86, 77, and 87, respectively. In certain embodiments, the VH of the Fab includes CDR1, CDR2, and CDR3 sequences of SEQ ID NOs: 80, 82, and 96, respectively, and the VL of the Fab includes CDR1, CDR2, and CDR3 sequences of SEQ ID NOs: 86, 77, and 87, respectively. In certain embodiments, the VH of the Fab includes CDR1, CDR2, and CDR3 sequences of SEQ ID NOs: 81, 82, and 97, respectively, and the VL of the Fab includes CDR1, CDR2, and CDR3 sequences of SEQ ID NOs: 86, 77, and 87, respectively.

In certain embodiments, the first antigen-binding site that binds NKG2D includes a VH with an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:98, and a VL with an amino acid sequence at least 90% (e.g., at least 91%, at least 92%, at least 93%, at least 94%, at least 95%, at least 96%, at least 97%, at least 98%, at least 99%, or 100%) identical to SEQ ID NO:85. In certain embodiments, the VH includes CDR1, CDR2, and CDR3 having the amino acid sequences of SEQ ID NOs: 80 or 81, 82, and 99 or 100, respectively (e.g., SEQ ID NOs: 80, 82, and 99, respectively, or SEQ ID NOs: 81, 82, and 100, respectively). In certain embodiments, the VL includes CDR1, CDR2, and CDR3 with the amino acid sequences of SEQ ID NOs: 86, 77, and 87, respectively. In certain embodiments, the first antigen-binding site includes (a) a VH with CDR1, CDR2, and CDR3 having the amino acid sequences of SEQ ID NOs: 80 or 81, 82, and 99 or 100, respectively (e.g., SEQ ID NOs: 80, 82, and 99, respectively, or SEQ ID NOs: 81, 82, and 100, respectively); and (b) a VL with CDR1, CDR2, and CDR3 having the amino acid sequences of SEQ ID NOs: 86, 77, and 87, respectively.

In certain embodiments, the first antigen-binding site that binds NKG2D includes a VH with an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:101, and a VL with an amino acid sequence at least 90% (e.g., at least 91%, at least 92%, at least 93%, at least 94%, at least 95%, at least 96%, at least 97%, at least 98%, at least 99%, or 100%) identical to SEQ ID NO:85. In certain embodiments, the VH includes CDR1, CDR2, and CDR3 with the amino acid sequences of SEQ ID NOs: 80 or 81, 82, and 102 or 103, respectively (e.g., SEQ ID NOs: 80, 82, and 102, respectively, or SEQ ID NOs: 81, 82, and 103, respectively). In certain embodiments, the VL includes CDR1, CDR2, and CDR3 with the amino acid sequences of SEQ ID NOs: 86, 77, and 87, respectively. In certain embodiments, the first antigen-binding site includes (a) a VH with CDR1, CDR2, and CDR3 having the amino acid sequences of SEQ ID NOs: 80 or 81, 82, and 102 or 103, respectively (e.g., SEQ ID NOs: 80, 82, and 102, respectively, or SEQ ID NOs: 81, 82, and 103, respectively); and (b) a VL that with CDR1, CDR2, and CDR3 having the amino acid sequences of SEQ ID NOs: 86, 77, and 87, respectively.

In certain embodiments, the first antigen-binding site that binds NKG2D includes a VH with an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:104, and a VL with an amino acid sequence at least 90% (e.g., at least 91%, at least 92%, at least 93%, at least 94%, at least 95%, at least 96%, at least 97%, at least 98%, at least 99%, or 100%) identical to SEQ ID NO:85. In certain embodiments, the VH includes CDR1, CDR2, and CDR3 with the amino acid sequences of SEQ ID NOs: 80 or 81, 82, and 105 or 106, respectively (e.g., SEQ ID NOs: 80, 82, and 105, respectively, or SEQ ID NOs: 81, 82, and 106, respectively). In certain embodiments, the VL includes CDR1, CDR2, and CDR3 with the amino acid sequences of SEQ ID NOs: 86, 77, and 87, respectively. In certain embodiments, the first antigen-binding site includes (a) a VH with CDR1, CDR2, and CDR3 having the amino acid sequences of SEQ ID NOs: 80 or 81, 82, and 105 or 106, respectively (e.g., SEQ ID NOs: 80, 82, and 105, respectively, or SEQ ID NOs: 81, 82, and 106, respectively); and (b) a VL with CDR1, CDR2, and CDR3 having the amino acid sequences of SEQ ID NOs: 86, 77, and 87, respectively.

In certain embodiments, the first antigen-binding site that binds NKG2D includes a VH with an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:107, and a VL with an amino acid sequence at least 90% (e.g., at least 91%, at least 92%, at least 93%, at least 94%, at least 95%, at least 96%, at least 97%, at least 98%, at least 99%, or 100%) identical to SEQ ID NO:85. In certain embodiments, the VH includes CDR1, CDR2, and CDR3 with the amino acid sequences of SEQ ID NOs: 80 or 81, 82, and 108 or 109, respectively (e.g., SEQ ID NOs: 80, 82, and 108, respectively, or SEQ ID NOs: 81, 82, and 109, respectively). In certain embodiments, the VL includes CDR1, CDR2, and CDR3 with the amino acid sequences of SEQ ID NOs: 86, 77, and 87, respectively. In certain embodiments, the first antigen-binding site includes (a) a VH with CDR1, CDR2, and CDR3 having the amino acid sequences of SEQ ID NOs: 80 or 81, 82, and 108 or 109, respectively (e.g., SEQ ID NOs: 80, 82, and 108, respectively, or SEQ ID NOs: 81, 82, and 109, respectively); and (b) a VL with CDR1, CDR2, and CDR3 having the amino acid sequences of SEQ ID NOs: 86, 77, and 87, respectively.

In certain embodiments, the first antigen-binding site that binds NKG2D includes a VH with an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:110, and a VL with an amino acid sequence at least 90% (e.g., at least 91%, at least 92%, at least 93%, at least 94%, at least 95%, at least 96%, at least 97%, at least 98%, at least 99%, or 100%) identical to SEQ ID NO:85. In certain embodiments, the first antigen-binding site includes a VH with a sequence of SEQ ID NO:110, and a VL with a sequence of SEQ ID NO:85. In certain embodiments, the VH includes CDR1, CDR2, and CDR3 with the amino acid sequences of SEQ ID NOs: 80 or 81, 82, and 111 or 112, respectively (e.g., SEQ ID NOs: 80, 82, and 111, respectively, or SEQ ID NOs: 81, 82, and 112, respectively). In certain embodiments, the VL includes CDR1, CDR2, and CDR3 with the amino acid sequences of SEQ ID NOs: 86, 77, and 87, respectively. In certain embodiments, the first antigen-binding site includes (a) a VH with CDR1, CDR2, and CDR3 having the amino acid sequences of SEQ ID NOs: 80 or 81, 82, and 111 or 112, respectively (e.g., SEQ ID NOs: 80, 82, and 111, respectively, or SEQ ID NOs: 81, 82, and 112, respectively); and (b) a VL with CDR1, CDR2, and CDR3 having the amino acid sequences of SEQ ID NOs: 86, 77, and 87, respectively. In certain embodiments, the first antigen-binding site includes a VH with the CDR1, CDR2, and CDR3 sequences of SEQ ID NOs: 80, 82, and 111, respectively, and a VL with the CDR1, CDR2, and CDR3 sequences of SEQ ID NOs: 86, 77, and 87, respectively. In certain embodiments, the first antigen-binding site includes a VH with the CDR1, CDR2, and CDR3 sequences of SEQ ID NOs: 81, 82, and 112, respectively, and a VL with the CDR1, CDR2, and CDR3 sequences of SEQ ID NOs: 86, 77, and 87, respectively.

In certain embodiments, the first antigen-binding site is a Fab that binds NKG2D and includes a VH with an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:110, and a VL with an amino acid sequence at least 90% (e.g., at least 91%, at least 92%, at least 93%, at least 94%, at least 95%, at least 96%, at least 97%, at least 98%, at least 99%, or 100%) identical to SEQ ID NO:85. In certain embodiments, the Fab includes a VH with the sequence of SEQ ID NO:110, and a VL with the sequence of SEQ ID NO:85. In certain embodiments, the VH of the Fab includes CDR1, CDR2, and CDR3 with the amino acid sequences of SEQ ID NOs: 80 or 81, 82, and 111 or 112, respectively (e.g., SEQ ID NOs: 80, 82, and 111, respectively, or SEQ ID NOs: 81, 82, and 112, respectively). In certain embodiments, the VL of the Fab includes CDR1, CDR2, and CDR3 with the amino acid sequences of SEQ ID NOs: 86, 77, and 87, respectively. In certain embodiments, the Fab includes (a) a VH that includes CDR1, CDR2, and CDR3 with the amino acid sequences of SEQ ID NOs: 80 or 81, 82, and 111 or 112, respectively (e.g., SEQ ID NOs: 80, 82, and 111, respectively, or SEQ ID NOs: 81, 82, and 112, respectively); and (b) a VL that includes CDR1, CDR2, and CDR3 with the amino acid sequences of SEQ ID NOs: 86, 77, and 87, respectively. In certain embodiments, the VH of the Fab includes CDR1, CDR2, and CDR3 sequences of SEQ ID NOs: 80, 82, and 111, respectively, and the VL of the Fab includes CDR1, CDR2, and CDR3 sequences of SEQ ID NOs: 86, 77, and 87, respectively. In certain embodiments, the VH of the Fab includes CDR1, CDR2, and CDR3 sequences of SEQ ID NOs: 81, 82, and 112, respectively, and the VL of the Fab includes CDR1, CDR2, and CDR3 sequences of SEQ ID NOs: 86, 77, and 87, respectively.

In certain embodiments, the first antigen-binding site that binds NKG2D includes a VH with an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:113, and a VL with an amino acid sequence at least 90% (e.g., at least 91%, at least 92%, at least 93%, at least 94%, at least 95%, at least 96%, at least 97%, at least 98%, at least 99%, or 100%) identical to SEQ ID NO:114.

In certain embodiments, the first antigen-binding site that binds NKG2D includes a VH with an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:116, and a VL with an amino acid sequence at least 90% (e.g., at least 91%, at least 92%, at least 93%, at least 94%, at least 95%, at least 96%, at least 97%, at least 98%, at least 99%, or 100%) identical to SEQ ID NO:117.

The multi-specific binding proteins can bind to NKG2D-expressing cells, which include but are not limited to NK cells, γδ T cells and CD8⁺ αβ T cells. Upon NKG2D binding, the multi-specific binding proteins may block natural ligands, such as ULBP6 and MICA, from binding to NKG2D and activating NK cells.

The multi-specific binding proteins binds to cells expressing CD16, an Fc receptor on the surface of leukocytes including natural killer cells, macrophages, neutrophils, eosinophils, mast cells, and follicular dendritic cells.

In some embodiments, a multi-specific binding protein described in the present disclosure binds to NKG2D with a binding affinity (K_{D}) of 1.00×10⁻⁴ mM to 10.0×10⁻⁴ mM, 1.50×10⁻⁴ mM to 10.0×10⁻⁴ mM, 2.00×10⁻⁴ mM to 10.0×10⁻⁴ mM, 2.50×10⁻⁴ mM to 10.0×10⁻⁴ mM, 3.00×10⁻⁴ mM to 10.0×10⁻⁴ mM, 3.50×10⁻⁴ mM to 10.0×10⁻⁴ mM, 4.00×10⁻⁴ mM to 10.0×10⁻⁴ mM, 4.50×10⁻⁴ mM to 10.0×10⁻⁴ mM, 5.00×10⁻⁴ mM to 10.0×10⁻⁴ mM, 5.50×10⁻⁴ mM to 10.0×10⁻⁴ mM, 6.00×10⁻⁴ mM to 10.0×10⁻⁴ mM, 6.5×10⁻⁴ mM to 10.0×10⁻⁴ mM, 7.00×10⁻⁴ mM to 10.0×10⁻⁴ mM, 7.5×10⁻⁴ mM to 10.0×10⁻⁴ mM, 8.00×10⁻⁴ mM to 10.0×10⁻⁴ mM, 8.5×10⁻⁴ mM to 10.0×10⁻⁴ mM, 9.00×10⁻⁴ mM to 10.0×10⁻⁴ mM, 9.5×10⁻⁴ mM to 10.0×10⁻⁴ mM, 1.00×10⁻⁴ mM to 9.00×10⁻⁴ mM, 1.00×10⁻⁴ mM to 8.00×10⁻⁴ mM, 1.00×10⁻⁴ mM to 7.00×10⁻⁴ mM, 1.00×10⁻⁴ mM to 6.00×10⁻⁴ mM, 1.00×10⁻⁴ mM to 5.00×10⁻⁴ mM, 1.00×10⁻⁴ mM to 4.00×10⁻⁴ mM, 1.00×10⁻⁴ mM to 3.00×10⁻⁴ mM, 1.00×10⁻⁴ mM to 2.00×10⁻⁴ mM, 5.00×10⁻⁴ mM to 8.00×10⁻⁴ mM, 5.50×10⁻⁴ mM to 8.00×10⁻⁴ mM, 6.00×10⁻⁴ mM to 8.00×10⁻⁴ mM, 6.5×10⁻⁴ mM to 8.00×10⁻⁴ mM, 7.00×10⁻⁴ mM to 8.00×10⁻⁴ mM, 7.5×10⁻⁴ mM to 8.00×10⁻⁴ mM, 1.50×10⁻⁴ mM to 6.00×10⁻⁴ mM, 2.00×10⁻⁴ mM to 6.00×10⁻⁴ mM, 2.50×10⁻⁴ mM to 6.00×10⁻⁴ mM, 3.00×10⁻⁴ mM to 6.00×10⁻⁴ mM, 3.50×10⁻⁴ mM to 6.00×10⁻⁴ mM, 4.00×10⁻⁴ mM to 6.00×10⁻⁴ mM, 4.50×10⁻⁴ mM to 6.00×10⁻⁴ mM, 5.00×10⁻⁴ mM to 6.00×10⁻⁴ mM, 5.50×10⁻⁴ mM to 6.00×10⁻⁴ mM, 1.50×10⁻⁴ mM to 4.00×10⁻⁴ mM, 2.00×10⁻⁴ mM to 4.00×10⁻⁴ mM, 2.50×10⁻⁴ mM to 4.00×10⁻⁴ mM, 3.00×10⁻⁴ mM to 4.00×10⁻⁴ mM, 3.50×10⁻⁴ mM to 4.00×10⁻⁴ mM, or 1.50×10⁻⁴ mM to 2.00×10⁻⁴ mM, as measured by surface plasmon resonance (SPR). In some embodiments, a multi-specific binding protein described in the present disclosure binds to NKG2D with a K_{D} of 4.00×10⁻⁴ mM to 6.00×10⁻⁴ mM, 4.50×10⁻⁴ mM to 6.00×10⁻⁴ mM, 5.00×10⁻⁴ mM to 6.00×10⁻⁴ mM, 5.50×10⁻⁴ mM to 6.00×10⁻⁴ mM, 4.00×10⁻⁴ mM to 5.80×10⁻⁴ mM, 4.50×10⁻⁴ mM to 5.80×10⁻⁴ mM, 5.00×10⁻⁴ mM to 5.80×10⁻⁴ mM, 5.50×10⁻⁴ mM to 5.80×10⁻⁴ mM, 4.00×10⁻⁴ mM to 5.6×10⁻⁴ mM, 4.50×10⁻⁴ mM to 5.6×10⁻⁴ mM, 5.00×10⁻⁴ mM to 5.6×10⁻⁴ mM, 5.50×10⁻⁴ mM to 5.6×10⁻⁴ mM, 4.00×10⁻⁴ mM to 5.4×10⁻⁴ mM, 4.50×10⁻⁴ mM to 5.4×10⁻⁴ mM, 5.00×10⁻⁴ mM to 5.4×10⁻⁴ mM, 4.00×10⁻⁴ mM to 5.2×10⁻⁴ mM, 4.50×10⁻⁴ mM to 5.2×10⁻⁴ mM, 5.00×10⁻⁴ mM to 5.2×10⁻⁴ mM, 4.00×10⁻⁴ mM to 5.0×10⁻⁴ mM, or 4.50×10⁻⁴ mM to 5.0×10⁻⁴ mM, as measured by SPR. In some embodiments, a multi-specific binding protein described in the present disclosure binds to NKG2D with a K_{D} of 4.50×10⁻⁴ mM to 5.20×10⁻⁴ mM, as measured by SPR. In some embodiments, a multi-specific binding protein described in the present disclosure binds to NKG2D with a K_{D} of about 4.8×10⁻⁴ mM, as measured by SPR.

In some embodiments, a multi-specific binding protein described in the present disclosure binds to NKG2D with an association rate constant of 1.00×10⁵ l/Ms to 10.0×10⁵ l/Ms, 1.50×10⁵ l/Ms to 10.0×10⁵ l/Ms, 2.00×10⁵ l/Ms to 10.0×10⁵ l/Ms, 2.50×10⁵ l/Ms to 10.0×10⁵ l/Ms, 3.00×10⁵ l/Ms to 10.0×10⁵ l/Ms, 3.50×10⁵ l/Ms to 10.0×10⁵ l/Ms, 4.00×10⁵ l/Ms to 10.0×10⁵ l/Ms, 4.50×10⁵ l/Ms to 10.0×10⁵ l/Ms, 5.00×10⁵ l/Ms to 10.0×10⁵ l/Ms, 5.50×10⁵ l/Ms to 10.0×10⁵ l/Ms, 6.00×10⁵ l/Ms to 10.0×10⁵ l/Ms, 6.5×10⁵ l/Ms to 10.0×10⁵ l/Ms, 7.00×10⁵ l/Ms to 10.0×10⁵ l/Ms, 7.5×10⁵ l/Ms to 10.0×10⁵ l/Ms, 8.00×10⁵ l/Ms to 10.0×10⁵ l/Ms, 8.5×10⁵ l/Ms to 10.0×10⁵ l/Ms, 9.00×10⁵ l/Ms to 10.0×10⁵ l/Ms, 9.5×10⁵ l/Ms to 10.0×10⁵ l/Ms, 1.00×10⁵ l/Ms to 9.00×10⁵ l/Ms, 1.00×10⁵ l/Ms to 8.00×10⁵ l/Ms, 1.00×10⁵ l/Ms to 7.00×10⁵ l/Ms, 1.00×10⁵ l/Ms to 6.00×10⁵ l/Ms, 1.00×10⁵ l/Ms to 5.00×10⁵ l/Ms, 1.00×10⁵ l/Ms to 4.00×10⁵ l/Ms, 1.00×10⁵ l/Ms to 3.00×10⁵ l/Ms, 1.00×10⁵ l/Ms to 2.00×10⁵ l/Ms, 5.00×10⁵ l/Ms to 8.00×10⁵ l/Ms 5.50×10⁵ l/Ms to 8.00×10⁵ l/Ms, 6.00×10⁵ l/Ms to 8.00×10⁵ l/Ms, 6.5×10⁵ l/Ms to 8.00×10⁵ l/Ms, 7.00×10⁵ l/Ms to 8.00×10⁵ l/Ms, 7.5×10⁵ l/Ms to 8.00×10⁵ l/Ms, 1.50×10⁵ l/Ms to 6.00×10⁵ l/Ms, 2.00×10⁵ l/Ms to 6.00×10⁵ l/Ms, 2.50×10⁵ l/Ms to 6.00×10⁵ l/Ms, 3.00×10⁵ l/Ms to 6.00×10⁵ l/Ms, 3.50×10⁵ l/Ms to 6.00×10⁵ l/Ms, 4.00×10⁵ l/Ms to 6.00×10⁵ l/Ms, 4.50×10⁵ l/Ms to 6.00×10⁵ l/Ms, 5.00×10⁵ l/Ms to 6.00×10⁵ l/Ms, 5.50×10⁵ l/Ms to 6.00×10⁵ l/Ms, 1.50×10⁵ l/Ms to 4.00×10⁵ l/Ms, 2.00×10⁵ l/Ms to 4.00×10⁵ l/Ms, 2.50×10⁵ l/Ms to 4.00×10⁵ l/Ms, 3.00×10⁵ l/Ms to 4.00×10⁵ l/Ms, 3.50×10⁵ l/Ms to 4.00×10⁵ l/Ms, or 1.50×10⁵ l/Ms to 2.00×10⁵ l/Ms, as measured by SPR. In some embodiments, a multi-specific binding protein described in the present disclosure binds to NKG2D with an association rate constant of 2.0×10⁵ l/Ms to 3.0×10⁵ l/Ms, 2.2×10⁵ l/Ms to 3.0×10⁵ l/Ms, 2.4×10⁵ 1/Ms to 3.0×10⁵ l/Ms, 2.6×10⁵ l/Ms to 3.0×10⁵ l/Ms, 2.8×10⁵ l/Ms to 3.0×10⁵ l/Ms, 2.0×10⁵ l/Ms to 2.8×10⁵ l/Ms, 2.2×10⁵ l/Ms to 2.8×10⁵ l/Ms, 2.4×10⁵ l/Ms to 2.8×10⁵ l/Ms, 2.6×10⁵ l/Ms to 2.8×10⁵ l/Ms, 2.0×10⁵ l/Ms to 2.6×10⁵ l/Ms, 2.2×10⁵ l/Ms to 2.6×10⁵ l/Ms, 2.4×10⁵ l/Ms to 2.6×10⁵ l/Ms, 2.0×10⁵ l/Ms to 2.4×10⁵ l/Ms, 2.2×10⁵ l/Ms to 2.4×10⁵ l/Ms, or 2.0×10⁵ l/Ms to 2.2×10⁵ l/Ms, as measured by SPR. In some embodiments, a multi-specific binding protein described in the present disclosure binds to NKG2D with an association rate constant of 2.0×10⁵ l/Ms to 2.6×10⁵ l/Ms, as measured by SPR. In some embodiments, a multi-specific binding protein described in the present disclosure binds to NKG2D with an association rate constant of about 2.3×10⁵ l/Ms, as measured by SPR.

In some embodiments, a multi-specific binding protein described in the present disclosure binds to NKG2D with a dissociation rate constant of 0.1×10⁻¹ l/s to 2.0×10⁻¹ l/s, 0.2×10⁻¹ l/s to 2.0×10⁻¹ l/s, 0.3×10⁻¹ l/s to 2.0×10⁻¹ l/s, 0.4×10⁻¹ l/s to 2.0×10⁻¹ l/s, 0.5×10⁻¹ l/s to 2.0×10⁻¹ l/s, 0.6×10⁻¹ l/s to 2.0×10⁻¹ l/s, 0.7×10⁻¹ l/s to 2.0×10⁻¹ l/s, 0.8×10⁻¹ l/s to 2.0×10⁻¹ l/s, 0.9×10⁻¹ l/s to 2.0×10⁻¹ l/s, 1.0×10⁻¹ l/s to 2.0×10⁻¹ l/s, 1.1×10⁻¹ l/s to 2.0×10⁻¹ l/s, 1.2×10⁻¹ l/s to 2.0×10⁻¹ l/s, 1.3×10⁻¹ l/s to 2.0×10⁻¹ l/s, 1.4×10⁻¹ l/s to 2.0×10⁻¹ l/s, 1.5×10⁻¹ l/s to 2.0×10⁻¹ l/s, 1.6×10⁻¹ l/s to 2.0×10⁻¹ l/s, 1.7×10⁻¹ l/s to 2.0×10⁻¹ l/s, 1.8×10⁻¹ l/s to 2.0×10⁻¹ l/s, 1.9×10⁻¹ l/s to 2.0×10⁻¹ l/s, 0.1×10⁻¹ l/s to 1.8×10⁻¹ l/s, 0.1×10⁻¹ l/s to 1.7×10⁻¹ l/s, 0.1×10⁻¹ l/s to 1.6×10⁻¹ l/s, 0.1×10⁻¹ l/s to 1.5×10⁻¹ l/s, 0.1×10⁻¹ l/s to 1.6×10⁻¹ l/s, 0.1×10⁻¹ l/s to 1.5×10⁻¹ l/s, 0.1×10⁻¹ l/s to 1.4×10⁻¹ l/s, 0.1×10⁻¹ l/s to 1.3×10⁻¹ l/s, 0.1×10⁻¹ l/s to 1.2×10⁻¹ l/s, 0.1×10⁻¹ l/s to 1.1×10⁻¹ l/s, 0.1×10⁻¹ l/s to 1.0×10⁻¹ l/s, 0.1×10⁻¹ l/s to 0.9×10⁻¹ l/s, 0.1×10⁻¹ l/s to 0.8×10⁻¹ l/s, 0.1×10⁻¹ l/s to 0.7×10⁻¹ l/s, 0.1×10⁻¹ l/s to 0.6×10⁻¹ l/s, 0.1×10⁻¹ l/s to 0.5×10⁻¹ l/s, 0.1×10⁻¹ l/s to 0.4×10⁻¹ l/s, 0.1×10⁻¹ l/s to 0.3×10⁻¹ l/s, 1.0×10⁻¹ l/s to 1.8×10⁻¹ l/s, 1.1×10⁻¹ l/s to 1.8×10⁻¹ l/s, 1.2×10⁻¹ l/s to 1.8×10⁻¹ l/s, 1.3×10⁻¹ l/s to 1.8×10⁻¹ l/s, 1.4×10⁻¹ l/s to 1.8×10⁻¹ l/s, 1.5×10⁻¹ l/s to 1.8×10⁻¹ l/s, 1.6×10⁻¹ l/s to 1.8×10⁻¹ l/s, 1.7×10⁻¹ l/s to 1.8×10⁻¹ l/s, 0.2×10⁻¹ l/s to 1.6×10⁻¹ l/s, 0.3×10⁻¹ l/s to 1.6×10⁻¹ l/s, 0.4×10⁻¹ l/s to 1.6×10⁻¹ l/s, 0.5×10⁻¹ l/s to 1.6×10⁻¹ l/s, 0.6×10⁻¹ l/s to 1.6×10⁻¹ l/s, 0.7×10⁻¹ l/s to 1.6×10⁻¹ l/s, 0.8×10⁻¹ l/s to 1.6×10⁻¹ l/s, 0.9×10⁻¹ l/s to 1.6×10⁻¹ l/s, 1.0×10⁻¹ l/s to 1.6×10⁻¹ l/s, 1.1×10⁻¹ l/s to 1.6×10⁻¹ l/s, 1.2×10⁻¹ l/s to 1.6×10⁻¹ l/s, 1.3×10⁻¹ l/s to 1.6×10⁻¹ l/s, 1.4×10⁻¹ l/s to 1.6×10⁻¹ l/s, 1.5×10⁻¹ l/s to 1.6×10⁻¹ l/s, 0.2×10⁻¹ l/s to 1.4×10⁻¹ l/s, 0.3×10⁻¹ l/s to 1.4×10⁻¹ l/s, 0.4×10⁻¹ l/s to 1.4×10⁻¹ l/s, 0.5×10⁻¹ l/s to 1.4×10⁻¹ l/s, 0.6×10⁻¹ l/s to 1.4×10⁻¹ l/s, 0.7×10⁻¹ l/s to 1.4×10⁻¹ l/s, 0.8×10⁻¹ l/s to 1.4×10⁻¹ l/s, 0.9×10⁻¹ l/s to 1.4×10⁻¹ l/s, 1.0×10⁻¹ l/s to 1.4×10⁻¹ l/s, 1.1×10⁻¹ l/s to 1.4×10⁻¹ l/s, 1.2×10⁻¹ l/s to 1.4×10⁻¹ l/s, 1.3×10⁻¹ l/s to 1.4×10⁻¹ l/s, 0.2×10⁻¹ l/s to 1.2×10⁻¹ l/s, 0.3×10⁻¹ l/s to 1.2×10⁻¹ l/s, 0.4×10⁻¹ l/s to 1.2×10⁻¹ l/s, 0.5×10⁻¹ l/s to 1.2×10⁻¹ l/s, 0.6×10⁻¹ l/s to 1.2×10⁻¹ l/s, 0.7×10⁻¹ l/s to 1.2×10⁻¹ l/s, 0.8×10⁻¹ l/s to 1.2×10⁻¹ l/s, 0.9×10⁻¹ l/s to 1.2×10⁻¹ l/s, 1.0×10⁻¹ l/s to 1.2×10⁻¹ l/s, 1.1×10⁻¹ l/s to 1.2×10⁻¹ l/s, 0.2×10⁻¹ l/s to 1.0×10⁻¹ l/s, 0.3×10⁻¹ l/s to 1.0×10⁻¹ l/s, 0.4×10⁻¹ l/s to 1.0×10⁻¹ l/s, 0.5×10⁻¹ l/s to 1.0×10⁻¹ l/s, 0.6×10⁻¹ l/s to 1.0×10⁻¹ l/s, 0.7×10⁻¹ l/s to 1.0×10⁻¹ l/s, 0.8×10⁻¹ l/s to 1.0×10⁻¹ l/s, or 0.9×10⁻¹ l/s to 1.0×10⁻¹ l/s, as measured by SPR. In some embodiments, a multi-specific binding protein described in the present disclosure binds to NKG2D with a dissociation rate constant of 0.2×10⁻¹ l/s to 1.8×10⁻¹ l/s, 0.4×10⁻¹ l/s to 1.8×10⁻¹ l/s, 0.6×10⁻¹ l/s to 1.8×10⁻¹ l/s, 0.8×10⁻¹ l/s to 1.8×10⁻¹ l/s, 0.2×10⁻¹ l/s to 1.6×10⁻¹ l/s, 0.4×10⁻¹ l/s to 1.6×10⁻¹ l/s, 0.6×10⁻¹ l/s to 1.6×10⁻¹ l/s, 0.8×10⁻¹ l/s to 1.6×10⁻¹ l/s, 1.0×10⁻¹ l/s to 1.6×10⁻¹ l/s, 0.2×10⁻¹ l/s to 1.4×10⁻¹ l/s, 0.4×10⁻¹ l/s to 1.4×10⁻¹ l/s, 0.6×10⁻¹ l/s to 1.4×10⁻¹ l/s, 0.8×10⁻¹ l/s to 1.4×10⁻¹ l/s, 1.0×10⁻¹ l/s to 1.4×10⁻¹ l/s, as measured by SPR. In some embodiments, a multi-specific binding protein described in the present disclosure binds to NKG2D with a dissociation rate constant of 0.6×10⁻¹ l/s to 1.6×10⁻¹ l/s, as measured by SPR. In some embodiments, a multi-specific binding protein described in the present disclosure binds to NKG2D with a dissociation rate constant of about 1.1×10⁻¹ l/s, as measured by SPR.

### EGFR Binding Site

In one aspect, the present disclosure provides multi-specific binding proteins that bind to the NKG2D receptor and CD16 receptor on natural killer cells, and EGFR on cancer cells, with an EGFR binding site incorporating a heavy chain variable domain (VH) and a light chain variable domain (VL) derived from panitumumab and having mutations in the VH and VL. Mutations that are contemplated, individually or in combination, in the VH and VL sequences described in the present disclosure include S62R in the VH, D92R in the VL, and/or F87Y in the VL, under the Chothia numbering scheme, which increase thermostability of the antigen-binding site while nevertheless retaining its binding affinity to EGFR. Table 2 lists some exemplary sequences of VH, VL, and scFv sequences that can bind to EGFR. CDR sequences are identified under Chothia numbering as indicated. Italicized sequence indicates a polypeptide linker.

TABLE 2
Sequences of Exemplary Antigen-Binding Sites that Bind EGFR

| | Heavy chain variable domain amino | Light chain variable domain amino |
|---|---|---|
| EGFR binder | acid sequence | acid sequence |
| EGFR-binder-1 | QVQLQESGPGLVKPSETLSLTCT | DIQMTQSPSSLSASVGDRVTIT |
| (panitumumab) | VSGGSVSSGDYYWTWIRQSPGK | CQASQDISNYLNWYQQKPGK |
| | GLEWIGHIYYSGNTNYNPSLKS | APKLLIYDASNLETGVPSRFSG |
| | RLTISIDTSKTQFSLKLSSVTAAD | SGSGTDFTFTISSLQPEDIATYF |
| | TAIYYCVRDRVTGAFDIWGQGT | CQHFDHLPLAFGGGTKVEIK |
| | MVTVSS | (SEQ ID NO: 139) |
| | (SEQ ID NO: 135) | CDR1: QASQDISNYLN (SEQ ID |
| | CDR1: GGSVSSGDYYWT (SEQ | NO: 140) |
| | ID NO: 136) | CDR2: LLIYDASNLET (SEQ ID |
| | CDR2: HIYYSGNTNYNPSLKS | NO: 141) |
| | (SEQ ID NO: 137) | CDR3: QHFDHLPLA (SEQ ID |
| | CDR3: DRVTGAFDI (SEQ ID | NO: 142) |
| | NO: 138) | |
| EGFR-scFv-1 | EGFR-scFv-1 (VL-VH): | |
| | DIQMTQSPSSLSASVGDRVTITCQASQDISNYLNWYQQKPGKAPK | |
| | LLIYDASNLETGVPSRFSGSGSGTDFTFTISSLQPEDIATYFCQHFD | |
| | HLPLAFGCGTKVEIKGGGGSGGGGSGGGGSGGGGSQVQLQESGP | |
| | GLVKPSETLSLTCTVSGGSVSSGDYYWTWIRQSPGKCLEWIGHIY | |
| | YSGNTNYNPSLKSRLTISIDTSKTQFSLKLSSVTAADTAIYYCVRDR | |
| | VTGAFDIWGQGTMVTVSS (SEQ ID NO: 143) | |
| | EGFR-scFv-1 (VH-VL): | |
| | QVQLQESGPGLVKPSETLSLTCTVSGGSVSSGDYYWTWIRQSPGK | |
| | CLEWIGHIYYSGNTNYNPSLKSRLTISIDTSKTQFSLKLSSVTAADT | |
| | AIYYCVRDRVTGAFDIWGQGTMVTVSSGGGGSGGGGSGGGGSGG | |
| | GGSDIQMTQSPSSLSASVGDRVTITCQASQDISNYLNWYQQKPGK | |
| | APKLLIYDASNLETGVPSRFSGSGSGTDFTFTISSLQPEDIATYFCQ | |
| | HFDHLPLAFGCGTKVEIK (SEQ ID NO: 144) | |
| EGFR-binder-2 | QVQLQESGPGLVKPSETLSLTCT | DIQMTQSPSSLSASVGDRVTIT |
| | VSGGSVSSGDYYWTWIRQSPGK | CQASQDISNYLNWYQQKPGK |
| | GLEWIGHIYYSGNTNYNPRLKS | APKLLIYDASNLETGVPSRFSG |
| | RLTISIDTSKTQFSLKLSSVTAAD | SGSGTDFTFTISSLQPEDIATYY |
| | TAIYYCVRDRVTGAFDIWGQGT | CQHFDHLPLAFGGGTKVEIK |
| | MVTVSS (SEQ ID NO: 145) | (SEQ ID NO: 147) |
| | CDR1: GGSVSSGDYYWT (SEQ | CDR1: QASQDISNYLN (SEQ ID |
| | ID NO: 136) | NO: 140) |
| | CDR2: HIYYSGNTNYNPRLKS | CDR2: LLIYDASNLET (SEQ ID |
| | (SEQ ID NO: 146) | NO: 141) |
| | CDR3: DRVTGAFDI (SEQ ID | CDR3: QHFDHLPLA (SEQ ID |
| | NO: 138) | NO: 142) |
| EGFR-scFv-2 | EGFR-scFv-2 (VL-VH): | |
| | DIQMTQSPSSLSASVGDRVTITCQASQDISNYLNWYQQKPGKAPK | |
| | LLIYDASNLETGVPSRFSGSGSGTDFTFTISSLQPEDIATYYCQHFD | |
| | HLPLAFGCGTKVEIKGGGGSGGGGSGGGGSGGGGSQVQLQESGP | |
| | GLVKPSETLSLTCTVSGGSVSSGDYYWTWIRQSPGKCLEWIGHIY | |
| | YSGNTNYNPRLKSRLTISIDTSKTQFSLKLSSVTAADTAIYYCVRD | |
| | RVTGAFDIWGQGTMVTVSS (SEQ ID NO: 148) | |
| | EGFR-scFv-2 (VH-VL): | |
| | QVQLQESGPGLVKPSETLSLTCTVSGGSVSSGDYYWTWIRQSPGK | |
| | CLEWIGHIYYSGNTNYNPRLKSRLTISIDTSKTQFSLKLSSVTAADT | |
| | AIYYCVRDRVTGAFDIWGQGTMVTVSSGGGGSGGGGSGGGGSGG | |
| | GGSDIQMTQSPSSLSASVGDRVTITCQASQDISNYLNWYQQKPGK | |
| | APKLLIYDASNLETGVPSRFSGSGSGTDFTFTISSLQPEDIATYYCQ | |
| | HFDHLPLAFGCGTKVEIK (SEQ ID NO: 149) | |
| EGFR-binder-3 | QVQLQESGPGLVKPSETLSLTCT | DIQMTQSPSSLSASVGDRVTIT |
| | VSGGSVSSGDYYWTWIRQSPGK | CQASQDISNYLNWYQQKPGK |
| | GLEWIGHIYYSGNTNYNPRLKS | APKLLIYDASNLETGVPSRFSG |
| | RLTISIDTSKTQFSLKLSSVTAAD | SGSGTDFTFTISSLQPEDIATYY |
| | TAIYYCVRDRVTGAFDIWGQGT | CQHFRHLPLAFGGGTKVEIK |
| | MVTVSS (SEQ ID NO: 145) | (SEQ ID NO: 150) |
| | CDR1: GGSVSSGDYYWT (SEQ | CDR1: QASQDISNYLN (SEQ |
| | ID NO: 136) | ID NO: 140) |
| | CDR2: HIYYSGNTNYNPRLKS | CDR2: LLIYDASNLET (SEQ ID |
| | (SEQ ID NO: 146) | NO: 141) |
| | CDR3: DRVTGAFDI (SEQ ID | CDR3: QHFRHLPLA (SEQ ID |
| | NO: 138) | NO: 151) |
| EGFR-binder-3 | QVQLQESGPGLVKPSETLSLTCT | DIQMTQSPSSLSASVGDRVTIT |
| (scFv mutations) | VSGGSVSSGDYYWTWIRQSPGK | CQASQDISNYLNWYQQKPGK |
| | CLEWIGHIYYSGNTNYNPRLKS | APKLLIYDASNLETGVPSRFSG |
| | RLTISIDTSKTQFSLKLSSVTAAD | SGSGTDFTFTISSLOPEDIATYY |
| | TAIYYCVRDRVTGAFDIWGQGT | CQHFRHLPLAFGCGTKVEIK |
| | MVTVSS (SEQ ID NO: 170) | (SEQ ID NO: 171) |
| | CDR1: GGSVSSGDYYWT (SEQ | CDR1: QASQDISNYLN (SEQ |
| | ID NO: 136) | ID NO:140) |
| | CDR2: HIYYSGNTNYNPRLKS | CDR2: LLIYDASNLET (SEQ ID |
| | (SEQ ID NO: 146) | NO:141) |
| | CDR3: DRVTGAFDI (SEQ ID | CDR3: QHFRHLPLA (SEQ ID |
| | NO: 138) | NO:151) |
| EGFR-scFv-3 | EGFR-scFv-3 (VL-VH): | |
| | DIQMTQSPSSLSASVGDRVTITCQASQDISNYLNWYQQKPGKAPK | |
| | LLIYDASNLETGVPSRFSGSGSGTDFTFTISSLQPEDIATYYCQHFR | |
| | HLPLAFGCGTKVEIKGGGGSGGGGSGGGGSGGGGSQVQLQESGP | |
| | GLVKPSETLSLTCTVSGGSVSSGDYYWTWIRQSPGKCLEWIGHIY | |
| | YSGNTNYNPRLKSRLTISIDTSKTQFSLKLSSVTAADTAIYYCVRD | |
| | RVTGAFDIWGQGTMVTVSS (SEQ ID NO: 152) | |
| | EGFR-scFv-3 (VH-VL): | |
| | QVQLQESGPGLVKPSETLSLTCTVSGGSVSSGDYYWTWIRQSPGK | |
| | CLEWIGHIYYSGNTNYNPRLKSRLTISIDTSKTQFSLKLSSVTAADT | |
| | AIYYCVRDRVTGAFDIWGQGTMVTVSSGGGGSGGGGSGGGGSGG | |
| | GGSDIQMTQSPSSLSASVGDRVTITCQASQDISNYLNWYQQKPGK | |
| | APKLLIYDASNLETGVPSRFSGSGSGTDFTFTISSLQPEDIATYYCQ | |
| | HFRHLPLAFGCGTKVEIK (SEQ ID NO: 153) | |
| EGFR-binder-4 | QVQLQESGPGLVKPSETLSLTCT | DIQMTQSPSSLSASVGDRVTIT |
| | VSGGSVSSGDYYWTWIRQSPGK | CQASQDISNYLNWYQQKPGK |
| | GLEWIGHIYYSGNTNYNPSLKS | APKLLIYDASNLETGVPSRFSG |
| | RLTISIDTSKTQFSLKLSSVTAAD | SGSGTDFTFTISSLQPEDIATYY |
| | TAIYYCVRDRVTGAFDIWGQGT | CQHFRHLPLAFGGGTKVEIK |
| | MVTVSS (SEQ ID NO: 135) | (SEQ ID NO: 150) |
| | CDR1: GGSVSSGDYYWT (SEQ | CDR1: QASQDISNYLN (SEQ |
| | ID NO: 136) | ID NO: 140) |
| | CDR2: HIYYSGNTNYNPSLKS | CDR2: LLIYDASNLET (SEQ ID |
| | (SEQ ID NO: 137) | NO: 141) |
| | CDR3: DRVTGAFDI (SEQ ID | CDR3: QHFRHLPLA (SEQ ID |
| | NO: 138) | NO: 151) |
| EGFR-scFv-4 | EGFR-scFv-4 (VL-VH): | |
| | DIQMTQSPSSLSASVGDRVTITCQASQDISNYLNWYQQKPGKAPK | |
| | LLIYDASNLETGVPSRFSGSGSGTDFTFTISSLQPEDIATYYCQHFR | |
| | HLPLAFGCGTKVEIKGGGGSGGGGSGGGGSGGGGSQVQLQESGP | |
| | GLVKPSETLSLTCTVSGGSVSSGDYYWTWIRQSPGKCLEWIGHIY | |
| | YSGNTNYNPSLKSRLTISIDTSKTQFSLKLSSVTAADTAIYYCVRDR | |
| | VTGAFDIWGQGTMVTVSS (SEQ ID NO: 154) | |
| | EGFR-scFv-4 (VH-VL): | |
| | QVQLQESGPGLVKPSETLSLTCTVSGGSVSSGDYYWTWIRQSPGK | |
| | CLEWIGHIYYSGNTNYNPSLKSRLTISIDTSKTQFSLKLSSVTAADT | |
| | AIYYCVRDRVTGAFDIWGQGTMVTVSSGGGGSGGGGSGGGGSGG | |
| | GGSDIQMTQSPSSLSASVGDRVTITCQASQDISNYLNWYQQKPGK | |
| | APKLLIYDASNLETGVPSRFSGSGSGTDFTFTISSLQPEDIATYYCQ | |
| | HFRHLPLAFGCGTKVEIK (SEQ ID NO: 155) | |
| Consensus | QVQLQESGPGLVKPSETLSLTCT | DIQMTQSPSSLSASVGDRVTIT |
| Sequence (EGFR- | VSGGSVSSGDYYWTWIRQSPGK | CQASQDISNYLNWYQQKPGK |
| binder-3 and | GLEWIGHIYYSGNTNYNPXLKS | APKLLIYDASNLETGVPSRFSG |
| EGFR-binder-4) | RLTISIDTSKTQFSLKLSSVTAAD | SGSGTDFTFTISSLQPEDIATYY |
| | TAIYYCVRDRVTGAFDIWGQGT | CQHFRHLPLAFGGGTKVEIK |
| | MVTVSS | (SEQ ID NO: 150) |
| | where X is R or S | CDR1: QASQDISNYLN (SEQ |
| | (SEQ ID NO: 156) | ID NO: 140) |
| | CDR1: GGSVSSGDYYWT (SEQ | CDR2: LLIYDASNLET (SEQ ID |
| | ID NO: 136) | NO: 141) |
| | CDR2: HIYYSGNTNYNPXLKS | CDR3: QHFRHLPLA (SEQ ID |
| | where X is R or S (SEQ ID NO: 157) | NO: 151) |
| | CDR3: DRVTGAFDI (SEQ ID | |
| | NO: 138) | |

| | |
|---|---|
| Consensus | scFv-309 (VL-VH): |
| Sequence (EGFR- | DIQMTQSPSSLSASVGDRVTITCQASQDISNYLNWYQQKPGKAPK |
| scFv-3 and | LLIYDASNLETGVPSRFSGSGSGTDFTFTISSLQPEDIATYYCQHFR |
| EGFR-scFv-4) | HLPLAFGCGTKVEIKGGGGSGGGGSGGGGSGGGGSQVQLQESGP |
| | GLVKPSETLSLTCTVSGGSVSSGDYYWTWIRQSPGKCLEWIGHIY |
| | YSGNTNYNPXLKSRLTISIDTSKTQFSLKLSSVTAADTAIYYCVRD |
| | RVTGAFDIWGQGTMVTVSS |
| | where X is R or S |
| | (SEQ ID NO: 158) |
| | scFv-310 (VH-VL): |
| | QVQLQESGPGLVKPSETLSLTCTVSGGSVSSGDYYWTWIRQSPGK |
| | CLEWIGHIYYSGNTNYNPXLKSRLTISIDTSKTQFSLKLSSVTAADT |
| | AIYYCVRDRVTGAFDIWGQGTMVTVSSGGGGSGGGGSGGGGSGG |
| | GGSDIQMTQSPSSLSASVGDRVTITCQASQDISNYLNWYQQKPGK |
| | APKLLIYDASNLETGVPSRFSGSGSGTDFTFTISSLQPEDIATYYCQ |
| | HFRHLPLAFGCGTKVEIK |
| | where X is R or S |
| | (SEQ ID NO: 159) |

In certain embodiments, the amino acid sequence of the second antigen-binding site includes an additional arginine (R) residue at the C-terminus of the VL (e.g., SEQ ID NOs: 139, 147, or 150). In certain embodiments, the second antigen-binding site includes an scFv including a VL amino acid sequence that contains an additional arginine (R) residue at the C-terminus of the VL amino acid sequence.

In certain embodiments, the amino acid sequence of the second antigen-binding site includes one or more mutations relative to the sequence of panitumumab selected from S62R in the VH, D92R in the VL, and F87Y in the VL, under the Chothia numbering scheme. In certain embodiments, the second antigen-binding site that binds EGFR (e.g., human EGFR) includes a VH with an amino acid sequence at least 90% (e.g., at least 91%, at least 92%, at least 93%, at least 94%, at least 95%, at least 96%, at least 97%, at least 98%, or at least 99%) identical to the VH of EGFR-binder-1, EGFR-binder-2, EGFR-binder-3, EGFR-binder-4, or the consensus sequence as disclosed in Table 2, and a VL with an amino acid sequence at least 90% (e.g., at least 91%, at least 92%, at least 93%, at least 94%, at least 95%, at least 96%, at least 97%, at least 98%, or at least 99%) identical to the VL of EGFR-binder-1, EGFR-binder-2, EGFR-binder-3, EGFR-binder-4, or the consensus sequence as disclosed in Table 2. In certain embodiments, the second antigen-binding site includes the heavy chain CDR1, CDR2, and CDR3 and the light chain CDR1, CDR2, and CDR3, determined under Kabat (see Kabat et al., (1991) Sequences of Proteins of Immunological Interest, NIH Publication No. 91-3242, Bethesda), Chothia (see, e.g., Chothia C & Lesk A M, (1987), J. Mol. Biol. 196: 901-917), MacCallum (see MacCallum R M et al., (1996) J. Mol. Biol. 262: 732-745), or any other CDR determination method known in the art, of the VH and VL CDR sequences of EGFR-binder-1, EGFR-binder-2, EGFR-binder-3, or EGFR-binder-4 as disclosed in Table 2. In certain embodiments, the second antigen-binding site includes the heavy chain CDR1, CDR2, and CDR3 and the light chain CDR1, CDR2, and CDR3 of EGFR-binder-1, EGFR-binder-2, EGFR-binder-3, or EGFR-binder-4 as disclosed in Table 2.

In certain embodiments, the second antigen-binding site that binds EGFR includes an scFv with a VH that has an amino acid sequence at least 90% (e.g., at least 91%, at least 92%, at least 93%, at least 94%, at least 95%, at least 96%, at least 97%, at least 98%, or at least 99%) identical to the VH of EGFR-binder-1, EGFR-binder-2, EGFR-binder-3, EGFR-binder-4, or the consensus sequence as disclosed in Table 2, and a VL that has an amino acid sequence at least 90% (e.g., at least 91%, at least 92%, at least 93%, at least 94%, at least 95%, at least 96%, at least 97%, at least 98%, or at least 99%) identical to the VL of EGFR-binder-1, EGFR-binder-2, EGFR-binder-3, EGFR-binder-4, or the consensus sequence as disclosed in Table 2. In certain embodiments, the second antigen-binding site includes an scFv with the heavy chain CDR1, CDR2, and CDR3 and the light chain CDR1, CDR2, and CDR3, determined under Kabat (see Kabat et al., (1991) Sequences of Proteins of Immunological Interest, NIH Publication No. 91-3242, Bethesda), Chothia (see, e.g., Chothia C & Lesk A M, (1987), J. Mol. Biol. 196: 901-917), MacCallum (see MacCallum R M et al., (1996) J. Mol. Biol. 262: 732-745), or any other CDR determination method known in the art, of the VH and VL CDR sequences of EGFR-binder-1, EGFR-binder-2, EGFR-binder-3, or EGFR-binder-4 as disclosed in Table 2.

In certain embodiments, the second antigen-binding site includes the heavy chain CDR1, CDR2, and CDR3 and the light chain CDR1, CDR2, and CDR3, determined under Kabat (see Kabat et al., (1991) Sequences of Proteins of Immunological Interest, NIH Publication No. 91-3242, Bethesda), Chothia (see, e.g., Chothia C & Lesk A M, (1987), J. Mol. Biol. 196: 901-917), MacCallum (see MacCallum R M et al., (1996) J. Mol. Biol. 262: 732-745), or any other CDR determination method known in the art, of the VH and VL CDR sequences of EGFR-binder-1 disclosed in Table 2. In certain embodiments, the VH includes CDR1, CDR2, and CDR3 with the amino acid sequences of SEQ ID NOs:136, 137, and 138, respectively. In certain embodiments, the VL includes CDR1, CDR2, and CDR3 with the amino acid sequences of SEQ ID NOs: 140, 141, and 142, respectively. In certain embodiments, the second antigen-binding site includes (i) a VH having CDR1, CDR2, and CDR3 sequences of SEQ ID NOs: 136, 137, and 138, respectively; and (ii) a VL having CDR1, CDR2, and CDR3 sequences of SEQ ID NOs: 140, 141, and 142, respectively. In certain embodiments, the second antigen-binding site includes a VH having an amino acid sequence at least 90% identical (e.g., at least 91%, at least 92%, at least 93%, at least 94%, at least 95%, at least 96%, at least 97%, at least 98%, or at least 99%) to the amino acid sequence of SEQ ID NO:135. In certain embodiments, the second antigen-binding site includes a VL having an amino acid sequence at least 90% identical (e.g., at least 91%, at least 92%, at least 93%, at least 94%, at least 95%, at least 96%, at least 97%, at least 98%, or at least 99%) to the amino acid sequence of the amino acid sequence of SEQ ID NO:139. In certain embodiments, the second antigen-binding site includes a VH having an amino acid sequence of SEQ ID NO:135. In certain embodiments, the second antigen-binding site includes a VL having an amino acid sequence of SEQ ID NO:139. In certain embodiments, the second antigen-binding site includes (i) a VH having a sequence at least 90% identical (e.g., at least 91%, at least 92%, at least 93%, at least 94%, at least 95%, at least 96%, at least 97%, at least 98%, or at least 99%) to the amino acid sequence of SEQ ID NO:135; and (ii) a VL having a sequence at least 90% identical (e.g., at least 91%, at least 92%, at least 93%, at least 94%, at least 95%, at least 96%, at least 97%, at least 98%, or at least 99%) to the amino acid sequence of SEQ ID NO:139. In certain embodiments, the second antigen-binding site includes (i) a VH having a sequence identical to the amino acid sequence of SEQ ID NO:135; and (ii) a VL having a sequence identical to the amino acid sequence of SEQ ID NO:139.

In certain embodiments, the second antigen-binding site is an scFv with a VH having CDR1, CDR2, and CDR3 sequences of SEQ ID NOs:136, 137, and 138, respectively. In certain embodiments, the second antigen-binding site is an scFv with a VL having CDR1, CDR2, and CDR3 sequences of SEQ ID NOs: 140, 141, and 142, respectively. In certain embodiments, the second antigen-binding site is an scFv with (i) a VH having CDR1, CDR2, and CDR3 sequences of SEQ ID NOs: 136, 137, and 138, respectively; and (ii) a VL having CDR1, CDR2, and CDR3 of SEQ ID NOs: 140, 141, and 142, respectively. In certain embodiments, the second antigen-binding site is an scFv including a VH having an amino acid sequence at least 90% identical (e.g., at least 91%, at least 92%, at least 93%, at least 94%, at least 95%, at least 96%, at least 97%, at least 98%, or at least 99%) to the amino acid sequence of SEQ ID NO:135. In certain embodiments, the second antigen-binding site is an scFv including a VL having an amino acid sequence at least 90% identical (e.g., at least 91%, at least 92%, at least 93%, at least 94%, at least 95%, at least 96%, at least 97%, at least 98%, or at least 99%) to the amino acid sequence of the amino acid sequence of SEQ ID NO:139. In certain embodiments, the second antigen-binding site is an scFv including a VH having the amino acid sequence of SEQ ID NO:135. In certain embodiments, the second antigen-binding site is an scFv including a VL having the amino acid sequence of SEQ ID NO:139. In certain embodiments, the second antigen-binding site is an scFv including (i) a VH with a sequence at least 90% identical (e.g., at least 91%, at least 92%, at least 93%, at least 94%, at least 95%, at least 96%, at least 97%, at least 98%, or at least 99%) to the amino acid sequence of SEQ ID NO:135; and (ii) a VL with a sequence at least 90% identical (e.g., at least 91%, at least 92%, at least 93%, at least 94%, at least 95%, at least 96%, at least 97%, at least 98%, or at least 99%) to the amino acid sequence of SEQ ID NO:139. In certain embodiments, the second antigen-binding site is an scFv including (i) a VH having a sequence of SEQ ID NO:135; and (ii) a VL having a sequence of SEQ ID NO:139. In certain embodiments, the second antigen-binding site is an scFv with an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 143 or 144. In certain embodiments, the second antigen-binding site is an scFv with an amino acid sequence identical to SEQ ID NO: 143 or 144. In certain embodiments, the second antigen-binding site is an scFv with an amino acid sequence identical to SEQ ID NO:143. In certain embodiments, the second antigen-binding site is an scFv with an amino acid sequence identical to SEQ ID NO:144.

In certain embodiments, the amino acid sequence of the second antigen-binding site incorporates mutations S62R in the VH and F87Y in the VL relative to the sequence of panitumumab, under the Chothia numbering scheme. In certain embodiments, the second antigen-binding site that binds EGFR includes a VH with an amino acid sequence at least 90% (e.g., at least 91%, at least 92%, at least 93%, at least 94%, at least 95%, at least 96%, at least 97%, at least 98%, or at least 99%) identical to SEQ ID NO:135, and a VL with an amino acid sequence at least 90% (e.g., at least 91%, at least 92%, at least 93%, at least 94%, at least 95%, at least 96%, at least 97%, at least 98%, or at least 99%) identical to SEQ ID NO:139. In certain embodiments, the second antigen-binding site that binds EGFR includes a VH with an amino acid sequence at least 90% (e.g., at least 91%, at least 92%, at least 93%, at least 94%, at least 95%, at least 96%, at least 97%, at least 98%, at least 99%, or 100%) identical to SEQ ID NO:145, and a VL with an amino acid sequence at least 90% (e.g., at least 91%, at least 92%, at least 93%, at least 94%, at least 95%, at least 96%, at least 97%, at least 98%, at least 99%, or 100%) identical to SEQ ID NO:147. In certain embodiments, the second antigen-binding site includes the heavy chain CDR1, CDR2, and CDR3 and the light chain CDR1, CDR2, and CDR3, determined under Kabat (see Kabat et al., (1991) Sequences of Proteins of Immunological Interest, NIH Publication No. 91-3242, Bethesda), Chothia (see, e.g., Chothia C & Lesk A M, (1987), J. Mol. Biol. 196: 901-917), MacCallum (see MacCallum R M et al., (1996) J. Mol. Biol. 262: 732-745), or any other CDR determination method known in the art, of the VH and VL CDR sequences of EGFR-binder-2 disclosed in Table 2. In certain embodiments, the VH includes CDR1, CDR2, and CDR3 sequences of SEQ ID NOs:136, 146, and 138, respectively. In certain embodiments, the VL includes CDR1, CDR2, and CDR3 sequences of SEQ ID NOs: 140, 141, and 142, respectively. In certain embodiments, the second antigen-binding site includes (i) a VH having CDR1, CDR2, and CDR3 sequences of SEQ ID NOs: 136, 146, and 138, respectively; and (ii) a VL having CDR1, CDR2, and CDR3 sequences of SEQ ID NOs: 140, 141, and 142, respectively. In certain embodiments, the second antigen-binding site includes a VH with an amino acid sequence at least 90% identical (e.g., at least 91%, at least 92%, at least 93%, at least 94%, at least 95%, at least 96%, at least 97%, at least 98%, or at least 99%) to the amino acid sequence of SEQ ID NO:145. In certain embodiments, the second antigen-binding site includes a VL with an amino acid sequence at least 90% identical (e.g., at least 91%, at least 92%, at least 93%, at least 94%, at least 95%, at least 96%, at least 97%, at least 98%, or at least 99%) to the amino acid sequence of SEQ ID NO:147. In certain embodiments, the second antigen-binding site includes a VH having the amino acid sequence of SEQ ID NO:145. In certain embodiments, the second antigen-binding site includes a VL having the amino acid sequence of SEQ ID NO:147. In certain embodiments, the second antigen-binding site includes (i) a VH having a sequence at least 90% identical (e.g., at least 91%, at least 92%, at least 93%, at least 94%, at least 95%, at least 96%, at least 97%, at least 98%, or at least 99%) to the amino acid sequence of SEQ ID NO:145; and (ii) a VL having a sequence at least 90% identical (e.g., at least 91%, at least 92%, at least 93%, at least 94%, at least 95%, at least 96%, at least 97%, at least 98%, or at least 99%) to the amino acid sequence of SEQ ID NO:147. In certain embodiments, the second antigen-binding site includes (i) a VH having a sequence identical to the amino acid sequence of SEQ ID NO:145; and (ii) a VL having a sequence identical to the amino acid sequence of SEQ ID NO:147.

In certain embodiments, the second antigen-binding site is an scFv that includes a VH having the CDR1, CDR2, and CDR3 sequences of SEQ ID NOs:136, 146, and 138, respectively. In certain embodiments, the second antigen-binding site is an scFv with a VL having the CDR1, CDR2, and CDR3 sequences of SEQ ID NOs: 140, 141, and 142, respectively. In certain embodiments, the second antigen-binding site is an scFv with (i) a VH having the CDR1, CDR2, and CDR3 sequences of SEQ ID NOs: 136, 146, and 138, respectively; and (ii) a VL having the CDR1, CDR2, and CDR3 of SEQ ID NOs: 140, 141, and 142, respectively. In certain embodiments, the second antigen-binding site is an scFv with a VH having an amino acid sequence at least 90% identical (e.g., at least 91%, at least 92%, at least 93%, at least 94%, at least 95%, at least 96%, at least 97%, at least 98%, or at least 99%) to the amino acid sequence of SEQ ID NO:145. In certain embodiments, the second antigen-binding site is an scFv including a VL having an amino acid sequence at least 90% identical (e.g., at least 91%, at least 92%, at least 93%, at least 94%, at least 95%, at least 96%, at least 97%, at least 98%, or at least 99%) to the amino acid sequence of SEQ ID NO:147. In certain embodiments, the second antigen-binding site is an scFv with a VH having the amino acid sequence of SEQ ID NO:145. In certain embodiments, the second antigen-binding site is an scFv with a VL having the amino acid sequence of SEQ ID NO:147. In certain embodiments, the second antigen-binding site is an scFv with (i) a VH that has a sequence at least 90% identical (e.g., at least 91%, at least 92%, at least 93%, at least 94%, at least 95%, at least 96%, at least 97%, at least 98%, or at least 99%) to the amino acid sequence of SEQ ID NO:145; and (ii) a VL that has a sequence at least 90% identical (e.g., at least 91%, at least 92%, at least 93%, at least 94%, at least 95%, at least 96%, at least 97%, at least 98%, or at least 99%) to the amino acid sequence of SEQ ID NO:147. In certain embodiments, the second antigen-binding site is an scFv with (i) a VH having the sequence of SEQ ID NO:145; and (ii) a VL having the sequence of SEQ ID NO:147. In certain embodiments, the second antigen-binding site is an scFv with an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 148 or 149. In certain embodiments, the second antigen-binding site is an scFv with an amino acid sequence identical to SEQ ID NO: 148 or 149. In certain embodiments, the second antigen-binding site is an scFv with an amino acid sequence identical to SEQ ID NO:148. In certain embodiments, the second antigen-binding site is an scFv with an amino acid sequence identical to SEQ ID NO:149.

In certain embodiments, the amino acid sequence of the second antigen-binding site incorporates mutations S62R in the VH, F87Y in the VL and D92R in the VL relative to the sequence of panitumumab, under the Chothia numbering scheme. In certain embodiments, the second antigen-binding site that binds EGFR includes a VH with an amino acid sequence at least 90% (e.g., at least 91%, at least 92%, at least 93%, at least 94%, at least 95%, at least 96%, at least 97%, at least 98%, or at least 99%) identical to SEQ ID NO:135, and a VL with an amino acid sequence at least 90% (e.g., at least 91%, at least 92%, at least 93%, at least 94%, at least 95%, at least 96%, at least 97%, at least 98%, or at least 99%) identical to SEQ ID NO:139. In certain embodiments, the second antigen-binding site that binds EGFR includes a VH with an amino acid sequence at least 90% (e.g., at least 91%, at least 92%, at least 93%, at least 94%, at least 95%, at least 96%, at least 97%, at least 98%, at least 99%, or 100%) identical to SEQ ID NO:145, and a VL with an amino acid sequence at least 90% (e.g., at least 91%, at least 92%, at least 93%, at least 94%, at least 95%, at least 96%, at least 97%, at least 98%, at least 99%, or 100%) identical to SEQ ID NO:150. In certain embodiments, the second antigen-binding site that binds EGFR includes a heavy chain variable domain (VH) including SEQ ID NO:170, and a light chain variable domain (VL) including SEQ ID NO:171. In certain embodiments, the second antigen-binding site includes the heavy chain CDR1, CDR2, and CDR3 and the light chain CDR1, CDR2, and CDR3, determined under Kabat (see Kabat et al., (1991) Sequences of Proteins of Immunological Interest, NIH Publication No. 91-3242, Bethesda), Chothia (see, e.g., Chothia C & Lesk A M, (1987), J. Mol. Biol. 196: 901-917), MacCallum (see MacCallum R M et al., (1996) J. Mol. Biol. 262: 732-745), or any other CDR determination method known in the art, of the VH and VL CDR sequences of EGFR-binder-3 disclosed in Table 2. In certain embodiments, the VH includes th CDR1, CDR2, and CDR3 sequences of SEQ ID NOs: 136, 146, and 138, respectively. In certain embodiments, the VL includes the CDR1, CDR2, and CDR3 sequences of SEQ ID NOs: 140, 141, and 151, respectively. In certain embodiments, the second antigen-binding site includes (i) a VH having the CDR1, CDR2, and CDR3 sequences of SEQ ID NOs: 136, 146, and 138, respectively; and (ii) a VL having the CDR1, CDR2, and CDR3 sequences of SEQ ID NOs: 140, 141, and 151, respectively. In certain embodiments, the second antigen-binding site includes a VH with an amino acid sequence at least 90% identical (e.g., at least 91%, at least 92%, at least 93%, at least 94%, at least 95%, at least 96%, at least 97%, at least 98%, or at least 99%) to the amino acid sequence of SEQ ID NO:145. In certain embodiments, the second antigen-binding site includes a VL with an amino acid sequence at least 90% identical (e.g., at least 91%, at least 92%, at least 93%, at least 94%, at least 95%, at least 96%, at least 97%, at least 98%, or at least 99%) to the amino acid sequence of the amino acid sequence of SEQ ID NO:150. In certain embodiments, the second antigen-binding site includes a VH having the amino acid sequence of SEQ ID NO:145. In certain embodiments, the second antigen-binding site includes a VL having the amino acid sequence of SEQ ID NO:150. In certain embodiments, the second antigen-binding site includes (i) a VH having a sequence at least 90% identical (e.g., at least 91%, at least 92%, at least 93%, at least 94%, at least 95%, at least 96%, at least 97%, at least 98%, or at least 99%) to the amino acid sequence of SEQ ID NO:145; and (ii) a VL having a sequence at least 90% identical (e.g., at least 91%, at least 92%, at least 93%, at least 94%, at least 95%, at least 96%, at least 97%, at least 98%, or at least 99%) to the amino acid sequence of SEQ ID NO:150. In certain embodiments, the second antigen-binding site includes (i) a VH having a sequence identical to the amino acid sequence of SEQ ID NO:145; and (ii) a VL having a sequence identical to the amino acid sequence of SEQ ID NO:150.

In certain embodiments, the second antigen-binding site includes a VH with an amino acid sequence at least 90% identical (e.g., at least 91%, at least 92%, at least 93%, at least 94%, at least 95%, at least 96%, at least 97%, at least 98%, or at least 99%) to the amino acid sequence of SEQ ID NO:170. In certain embodiments, the second antigen-binding site includes a VL with an amino acid sequence at least 90% identical (e.g., at least 91%, at least 92%, at least 93%, at least 94%, at least 95%, at least 96%, at least 97%, at least 98%, or at least 99%) to the amino acid sequence of the amino acid sequence of SEQ ID NO:171. In certain embodiments, the second antigen-binding site includes a VH having the amino acid sequence of SEQ ID NO:170. In certain embodiments, the second antigen-binding site includes a VL having the amino acid sequence of SEQ ID NO:171. In certain embodiments, the second antigen-binding site includes (i) a VH having a sequence at least 90% identical (e.g., at least 91%, at least 92%, at least 93%, at least 94%, at least 95%, at least 96%, at least 97%, at least 98%, or at least 99%) to the amino acid sequence of SEQ ID NO:170; and (ii) a VL having a sequence at least 90% identical (e.g., at least 91%, at least 92%, at least 93%, at least 94%, at least 95%, at least 96%, at least 97%, at least 98%, or at least 99%) to the amino acid sequence of SEQ ID NO:171. In certain embodiments, the second antigen-binding site includes (i) a VH having a sequence identical to the amino acid sequence of SEQ ID NO:170; and (ii) a VL having a sequence identical to the amino acid sequence of SEQ ID NO:171.

In certain embodiments, the second antigen-binding site is an scFv including a VH having the CDR1, CDR2, and CDR3 sequences of SEQ ID NOs:136, 146, and 138, respectively. In certain embodiments, the second antigen-binding site is an scFv including a VL having the CDR1, CDR2, and CDR3 sequences of SEQ ID NOs: 140, 141, and 151, respectively. In certain embodiments, the second antigen-binding site is an scFv including (i) a VH having the CDR1, CDR2, and CDR3 sequences of SEQ ID NOs: 136, 146, and 138, respectively; and (ii) a VL having the CDR1, CDR2, and CDR3 of SEQ ID NOs: 140, 141, and 151, respectively. In certain embodiments, the second antigen-binding site is an scFv including a VH having an amino acid sequence at least 90% identical (e.g., at least 91%, at least 92%, at least 93%, at least 94%, at least 95%, at least 96%, at least 97%, at least 98%, or at least 99%) to the amino acid sequence of SEQ ID NO:170. In certain embodiments, the second antigen-binding site is an scFv including a VL having an amino acid sequence at least 90% identical (e.g., at least 91%, at least 92%, at least 93%, at least 94%, at least 95%, at least 96%, at least 97%, at least 98%, or at least 99%) to the amino acid sequence of the amino acid sequence of SEQ ID NO:171. In certain embodiments, the second antigen-binding site is an scFv including a VH having an amino acid sequence of SEQ ID NO:170. In certain embodiments, the second antigen-binding site is an scFv including a VL having an amino acid sequence of SEQ ID NO:171. In certain embodiments, the second antigen-binding site is an scFv including (i) a VH with a sequence at least 90% identical (e.g., at least 91%, at least 92%, at least 93%, at least 94%, at least 95%, at least 96%, at least 97%, at least 98%, or at least 99%) to the amino acid sequence of SEQ ID NO:170; and (ii) a VL with a sequence at least 90% identical (e.g., at least 91%, at least 92%, at least 93%, at least 94%, at least 95%, at least 96%, at least 97%, at least 98%, or at least 99%) to the amino acid sequence of SEQ ID NO:171. In certain embodiments, the second antigen-binding site is an scFv including (i) a VH having the sequence of SEQ ID NO:170; and (ii) a VL having the sequence of SEQ ID NO:171. In certain embodiments, the second antigen-binding site is an scFv with an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 152 or 153. In certain embodiments, the second antigen-binding site is an scFv with an amino acid sequence identical to SEQ ID NO: 152 or 153. In certain embodiments, the second antigen-binding site is an scFv with an amino acid sequence identical to SEQ ID NO:152. In certain embodiments, the second antigen-binding site is an scFv with an amino acid sequence identical to SEQ ID NO:153.

In certain embodiments, the amino acid sequence of the second antigen-binding site incorporates mutations F87Y in the VL and D92R in the VL relative to the sequence of panitumumab, under the Chothia numbering scheme. In certain embodiments, the second antigen-binding site that binds EGFR includes a VH with an amino acid sequence at least 90% (e.g., at least 91%, at least 92%, at least 93%, at least 94%, at least 95%, at least 96%, at least 97%, at least 98%, or at least 99%) identical to SEQ ID NO:135, and a VL with an amino acid sequence at least 90% (e.g., at least 91%, at least 92%, at least 93%, at least 94%, at least 95%, at least 96%, at least 97%, at least 98%, or at least 99%) identical to SEQ ID NO:139. In certain embodiments, the second antigen-binding site that binds EGFR includes a VH with an amino acid sequence at least 90% (e.g., at least 91%, at least 92%, at least 93%, at least 94%, at least 95%, at least 96%, at least 97%, at least 98%, at least 99%, or 100%) identical to SEQ ID NO:135, and a VL with an amino acid sequence at least 90% (e.g., at least 91%, at least 92%, at least 93%, at least 94%, at least 95%, at least 96%, at least 97%, at least 98%, at least 99%, or 100%) identical to SEQ ID NO:150. In certain embodiments, the second antigen-binding site includes the heavy chain CDR1, CDR2, and CDR3 and the light chain CDR1, CDR2, and CDR3, determined under Kabat (see Kabat et al., (1991) Sequences of Proteins of Immunological Interest, NIH Publication No. 91-3242, Bethesda), Chothia (see, e.g., Chothia C & Lesk A M, (1987), J. Mol. Biol. 196: 901-917), MacCallum (see MacCallum R M et al., (1996) J. Mol. Biol. 262: 732-745), or any other CDR determination method known in the art, of the VH and VL CDR sequences of EGFR-binder-4 disclosed in Table 2. In certain embodiments, the VH includes the CDR1, CDR2, and CDR3 sequences of SEQ ID NOs: 136, 137, and 138, respectively. In certain embodiments, the VL includes th CDR1, CDR2, and CDR3 sequences of SEQ ID NOs: 140, 141, and 151, respectively. In certain embodiments, the second antigen-binding site includes (i) a VH having the CDR1, CDR2, and CDR3 sequences of SEQ ID NOs: 136, 137, and 138, respectively; and (ii) a VL having the CDR1, CDR2, and CDR3 sequences of SEQ ID NOs: 140, 141, and 151, respectively. In certain embodiments, the second antigen-binding site includes a VH with an amino acid sequence at least 90% identical (e.g., at least 91%, at least 92%, at least 93%, at least 94%, at least 95%, at least 96%, at least 97%, at least 98%, or at least 99%) to the amino acid sequence of SEQ ID NO:135. In certain embodiments, the second antigen-binding site includes a VL with an amino acid sequence at least 90% identical (e.g., at least 91%, at least 92%, at least 93%, at least 94%, at least 95%, at least 96%, at least 97%, at least 98%, or at least 99%) to the amino acid sequence of SEQ ID NO:150. In certain embodiments, the second antigen-binding site includes a VH having the amino acid sequence of SEQ ID NO:135. In certain embodiments, the second antigen-binding site includes a VL having the amino acid sequence of SEQ ID NO:150. In certain embodiments, the second antigen-binding site includes (i) a VH having a sequence at least 90% identical (e.g., at least 91%, at least 92%, at least 93%, at least 94%, at least 95%, at least 96%, at least 97%, at least 98%, or at least 99%) to the amino acid sequence of SEQ ID NO:135; and (ii) a VL having a sequence at least 90% identical (e.g., at least 91%, at least 92%, at least 93%, at least 94%, at least 95%, at least 96%, at least 97%, at least 98%, or at least 99%) to the amino acid sequence of SEQ ID NO:150. In certain embodiments, the second antigen-binding site includes (i) a VH having a sequence identical to the amino acid sequence of SEQ ID NO:135; and (ii) a VL having a sequence identical to the amino acid sequence of SEQ ID NO:150.

In certain embodiments, the second antigen-binding site is an scFv including a VH having CDR1, CDR2, and CDR3 sequences of SEQ ID NOs:136, 137, and 138, respectively. In certain embodiments, the second antigen-binding site is an scFv including a VL having CDR1, CDR2, and CDR3 sequences of SEQ ID NOs: 140, 141, and 151, respectively. In certain embodiments, the second antigen-binding site is an scFv including (i) a VH having CDR1, CDR2, and CDR3 sequences of SEQ ID NOs: 136, 137, and 138, respectively; and (ii) a VL having CDR1, CDR2, and CDR3 of SEQ ID NOs: 140, 141, and 151, respectively. In certain embodiments, the second antigen-binding site is an scFv including a VH having an amino acid sequence at least 90% identical (e.g., at least 91%, at least 92%, at least 93%, at least 94%, at least 95%, at least 96%, at least 97%, at least 98%, or at least 99%) to the amino acid sequence of SEQ ID NO:135. In certain embodiments, the second antigen-binding site is an scFv including a VL having an amino acid sequence at least 90% identical (e.g., at least 91%, at least 92%, at least 93%, at least 94%, at least 95%, at least 96%, at least 97%, at least 98%, or at least 99%) to the amino acid sequence of the amino acid sequence of SEQ ID NO:150. In certain embodiments, the second antigen-binding site is an scFv including a VH having an amino acid sequence of SEQ ID NO:135. In certain embodiments, the second antigen-binding site is an scFv including a VL having an amino acid sequence of SEQ ID NO:150. In certain embodiments, the second antigen-binding site is an scFv including (i) a VH that includes a sequence at least 90% identical (e.g., at least 91%, at least 92%, at least 93%, at least 94%, at least 95%, at least 96%, at least 97%, at least 98%, or at least 99%) to the amino acid sequence of SEQ ID NO:135; and (ii) a VL that includes a sequence at least 90% identical (e.g., at least 91%, at least 92%, at least 93%, at least 94%, at least 95%, at least 96%, at least 97%, at least 98%, or at least 99%) to the amino acid sequence of SEQ ID NO:150. In certain embodiments, the second antigen-binding site is an scFv including (i) a VH having a sequence of SEQ ID NO:135; and (ii) a VL having a sequence of SEQ ID NO:150. In certain embodiments, the second antigen-binding site is an scFv including an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 154 or 155. In certain embodiments, the second antigen-binding site is an scFv including an amino acid sequence identical to SEQ ID NO: 154 or 155. In certain embodiments, the second antigen-binding site is an scFv including an amino acid sequence identical to SEQ ID NO:154. In certain embodiments, the second antigen-binding site an scFv including an amino acid sequence identical to SEQ ID NO:155.

In certain embodiments, the amino acid sequence of the second antigen-binding site incorporates F87Y in the VL and D92R in the VL, and optionally S62R in the VH mutations relative to the sequence of panitumumab, under the Chothia numbering scheme. In certain embodiments, the second antigen-binding site that binds EGFR includes a VH having an amino acid sequence at least 90% (e.g., at least 91%, at least 92%, at least 93%, at least 94%, at least 95%, at least 96%, at least 97%, at least 98%, or at least 99%) identical to SEQ ID NO:135, and a VL having an amino acid sequence at least 90% (e.g., at least 91%, at least 92%, at least 93%, at least 94%, at least 95%, at least 96%, at least 97%, at least 98%, or at least 99%) identical to SEQ ID NO:139. In certain embodiments, the second antigen-binding site that binds EGFR includes a VH with an amino acid sequence at least 90% (e.g., at least 91%, at least 92%, at least 93%, at least 94%, at least 95%, at least 96%, at least 97%, at least 98%, at least 99%, or 100%) identical to SEQ ID NO:156, and a VL with an amino acid sequence at least 90% (e.g., at least 91%, at least 92%, at least 93%, at least 94%, at least 95%, at least 96%, at least 97%, at least 98%, at least 99%, or 100%) identical to SEQ ID NO:150. In certain embodiments, the second antigen-binding site includes the heavy chain CDR1, CDR2, and CDR3 and the light chain CDR1, CDR2, and CDR3, determined under Kabat (see Kabat et al., (1991) Sequences of Proteins of Immunological Interest, NIH Publication No. 91-3242, Bethesda), Chothia (see, e.g., Chothia C & Lesk A M, (1987), J. Mol. Biol. 196: 901-917), MacCallum (see MacCallum R M et al., (1996) J. Mol. Biol. 262: 732-745), or any other CDR determination method known in the art, of the consensus VH and VL CDR sequences disclosed in Table 2. In certain embodiments, the VH includes the CDR1, CDR2, and CDR3 sequences of SEQ ID NOs: 136, 157, and 138, respectively. In certain embodiments, the VL includes the CDR1, CDR2, and CDR3 sequences of SEQ ID NOs: 140, 141, and 151, respectively. In certain embodiments, the second antigen-binding site includes (i) a VH having the CDR1, CDR2, and CDR3 sequences of SEQ ID NOs: 136, 157, and 138, respectively; and (ii) a VL having the CDR1, CDR2, and CDR3 sequences of SEQ ID NOs: 140, 141, and 151, respectively. In certain embodiments, the second antigen-binding site includes a VH with an amino acid sequence at least 90% identical (e.g., at least 91%, at least 92%, at least 93%, at least 94%, at least 95%, at least 96%, at least 97%, at least 98%, or at least 99%) to the amino acid sequence of SEQ ID NO:156. In certain embodiments, the second antigen-binding site includes a VL having an amino acid sequence at least 90% identical (e.g., at least 91%, at least 92%, at least 93%, at least 94%, at least 95%, at least 96%, at least 97%, at least 98%, or at least 99%) to the amino acid sequence of the amino acid sequence of SEQ ID NO:150. In certain embodiments, the second antigen-binding site includes a VH having the amino acid sequence of SEQ ID NO:156. In certain embodiments, the second antigen-binding site includes a VL having the amino acid sequence of SEQ ID NO:150. In certain embodiments, the second antigen-binding site includes (i) a VH having a sequence at least 90% identical (e.g., at least 91%, at least 92%, at least 93%, at least 94%, at least 95%, at least 96%, at least 97%, at least 98%, or at least 99%) to the amino acid sequence of SEQ ID NO:156; and (ii) a VL having a sequence at least 90% identical (e.g., at least 91%, at least 92%, at least 93%, at least 94%, at least 95%, at least 96%, at least 97%, at least 98%, or at least 99%) to the amino acid sequence of SEQ ID NO:150. In certain embodiments, the second antigen-binding site includes (i) a VH having a sequence identical to the amino acid sequence of SEQ ID NO:156; and (ii) a VL having a sequence identical to the amino acid sequence of SEQ ID NO:150.

In certain embodiments, the second antigen-binding site is an scFv including a VH having the CDR1, CDR2, and CDR3 sequences of SEQ ID NOs:136, 157, and 138, respectively. In certain embodiments, the second antigen-binding site is an scFv including a VL having the CDR1, CDR2, and CDR3 sequences of SEQ ID NOs: 140, 141, and 142, respectively. In certain embodiments, the second antigen-binding site is an scFv including (i) a VH having the CDR1, CDR2, and CDR3 sequences of SEQ ID NOs: 136, 157, and 138, respectively; and (ii) a VL having the CDR1, CDR2, and CDR3 sequences of SEQ ID NOs: 140, 141, and 142, respectively. In certain embodiments, the second antigen-binding site is an scFv including a VH having an amino acid sequence at least 90% identical (e.g., at least 91%, at least 92%, at least 93%, at least 94%, at least 95%, at least 96%, at least 97%, at least 98%, or at least 99%) to the amino acid sequence of SEQ ID NO:156. In certain embodiments, the second antigen-binding site is an scFv including a VL having an amino acid sequence at least 90% identical (e.g., at least 91%, at least 92%, at least 93%, at least 94%, at least 95%, at least 96%, at least 97%, at least 98%, or at least 99%) to the amino acid sequence of SEQ ID NO:150. In certain embodiments, the second antigen-binding site is an scFv including a VH having the amino acid sequence of SEQ ID NO:156. In certain embodiments, the second antigen-binding site is an scFv including a VL having the amino acid sequence of SEQ ID NO:150. In certain embodiments, the second antigen-binding site is an scFv including (i) a VH with a sequence at least 90% identical (e.g., at least 91%, at least 92%, at least 93%, at least 94%, at least 95%, at least 96%, at least 97%, at least 98%, or at least 99%) to the amino acid sequence of SEQ ID NO:156; and (ii) a VL with a sequence at least 90% identical (e.g., at least 91%, at least 92%, at least 93%, at least 94%, at least 95%, at least 96%, at least 97%, at least 98%, or at least 99%) to the amino acid sequence of SEQ ID NO:150. In certain embodiments, the second antigen-binding site is an scFv including (i) a VH having the sequence of SEQ ID NO:156; and (ii) a VL having the sequence of SEQ ID NO:150. In certain embodiments, the second antigen-binding site is an scFv including an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 158 or 159. In certain embodiments, the second antigen-binding site is an scFv with an amino acid sequence identical to SEQ ID NO: 158 or 159. In certain embodiments, the second antigen-binding site is an scFv with an amino acid sequence identical to SEQ ID NO:158. In certain embodiments, the second antigen-binding site is an scFv with an amino acid sequence identical to SEQ ID NO:159.

Alternatively, novel antigen-binding sites that can bind to EGFR can be identified by screening for binding to the amino acid sequence of EGFR, an isoform thereof, a variant thereof, a mature extracellular fragment thereof or a fragment containing a domain of EGFR.

TABLE 3
Sequences for exemplary EGFR Isoforms

| EGFR Isoform | Amino Acid Sequence |
|---|---|
| 1 | MRPSGTAGAALLALLAALCPASRALEEKKVCQGTSNKLTQLGTFE |
| (Uniprot ID No.: | DHFLSLQRMFNNCEVVLGNLEITYVQRNYDLSFLKTIQEVAGYVL |
| P00533-1) | IALNTVERIPLENLQIIRGNMYYENSYALAVLSNYDANKTGLKELP |
| | MRNLQEILHGAVRFSNNPALCNVESIQWRDIVSSDFLSNMSMDFQ |
| | NHLGSCQKCDPSCPNGSCWGAGEENCQKLTKIICAQQCSGRCRG |
| | KSPSDCCHNQCAAGCTGPRESDCLVCRKFRDEATCKDTCPPLML |
| | YNPTTYQMDVNPEGKYSFGATCVKKCPRNYVVTDHGSCVRACG |
| | ADSYEMEEDGVRKCKKCEGPCRKVCNGIGIGEFKDSLSINATNIK |
| | HFKNCTSISGDLHILPVAFRGDSFTHTPPLDPQELDILKTVKEITGFL |
| | LIQAWPENRTDLHAFENLEIIRGRTKQHGQFSLAVVSLNITSLGLRS |
| | LKEISDGDVIISGNKNLCYANTINWKKLFGTSGQKTKIISNRGENSC |
| | KATGQVCHALCSPEGCWGPEPRDCVSCRNVSRGRECVDKCNLLE |
| | GEPREFVENSECIQCHPECLPQAMNITCTGRGPDNCIQCAHYIDGP |
| | HCVKTCPAGVMGENNTLVWKYADAGHVCHLCHPNCTYGCTGP |
| | GLEGCPTNGPKIPSIATGMVGALLLLLVVALGIGLFMRRRHIVRKR |
| | TLRRLLQERELVEPLTPSGEAPNQALLRILKETEFKKIKVLGSGAFG |
| | TVYKGLWIPEGEKVKIPVAIKELREATSPKANKEILDEAYVMASV |
| | DNPHVCRLLGICLTSTVQLITQLMPFGCLLDYVREHKDNIGSQYLL |
| | NWCVQIAKGMNYLEDRRLVHRDLAARNVLVKTPQHVKITDFGL |
| | AKLLGAEEKEYHAEGGKVPIKWMALESILHRIYTHQSDVWSYGV |
| | TVWELMTFGSKPYDGIPASEISSILEKGERLPQPPICTIDVYMIMVK |
| | CWMIDADSRPKFRELIIEFSKMARDPQRYLVIQGDERMHLPSPTDS |
| | NFYRALMDEEDMDDVVDADEYLIPQQGFFSSPSTSRTPLLSSLSAT |
| | SNNSTVACIDRNGLQSCPIKEDSFLQRYSSDPTGALTEDSIDDTFLP |
| | VPEYINQSVPKRPAGSVQNPVYHNQPLNPAPSRDPHYQDPHSTAV |
| | GNPEYLNTVQPTCVNSTFDSPAHWAQKGSHQISLDNPDYQQDFFP |
| | KEAKPNGIFKGSTAENAEYLRVAPQSSEFIGA |
| | (SEQ ID NO: 160) |
| 2 | MRPSGTAGAALLALLAALCPASRALEEKKVCQGTSNKLTQLGTFE |
| (Uniprot ID No.: | DHFLSLQRMFNNCEVVLGNLEITYVQRNYDLSFLKTIQEVAGYVL |
| P00533-2) | IALNTVERIPLENLQIIRGNMYYENSYALAVLSNYDANKTGLKELP |
| | MRNLQEILHGAVRFSNNPALCNVESIQWRDIVSSDFLSNMSMDFQ |
| | NHLGSCQKCDPSCPNGSCWGAGEENCQKLTKIICAQQCSGRCRG |
| | KSPSDCCHNQCAAGCTGPRESDCLVCRKFRDEATCKDTCPPLML |
| | YNPTTYQMDVNPEGKYSFGATCVKKCPRNYVVTDHGSCVRACG |
| | ADSYEMEEDGVRKCKKCEGPCRKVCNGIGIGEFKDSLSINATNIK |
| | HFKNCTSISGDLHILPVAFRGDSFTHTPPLDPQELDILKTVKEITGLS |
| | (SEQ ID NO: 161) |
| 3 | MRPSGTAGAALLALLAALCPASRALEEKKVCQGTSNKLTQLGTFE |
| (Uniprot ID No.: | DHFLSLQRMFNNCEVVLGNLEITYVQRNYDLSFLKTIQEVAGYVL |
| P00533-3) | IALNTVERIPLENLQUIRGNMYYENSYALAVLSNYDANKTGLKELP |
| | MRNLQEILHGAVRFSNNPALCNVESIQWRDIVSSDFLSNMSMDFQ |
| | NHLGSCQKCDPSCPNGSCWGAGEENCQKLTKIICAQQCSGRCRG |
| | KSPSDCCHNQCAAGCTGPRESDCLVCRKFRDEATCKDTCPPLML |
| | YNPTTYQMDVNPEGKYSFGATCVKKCPRNYVVTDHGSCVRACG |
| | ADSYEMEEDGVRKCKKCEGPCRKVCNGIGIGEFKDSLSINATNIK |
| | HFKNCTSISGDLHILPVAFRGDSFTHTPPLDPQELDILKTVKEITGFL |
| | LIQAWPENRTDLHAFENLEIIRGRTKQHGQFSLAVVSLNITSLGLRS |
| | LKEISDGDVIISGNKNLCYANTINWKKLFGTSGQKTKIISNRGENSC |
| | KATGQVCHALCSPEGCWGPEPRDCVSCRNVSRGRECVDKCNLLE |
| | GEPREFVENSECIQCHPECLPQAMNITCTGRGPDNCIQCAHYIDGP |
| | HCVKTCPAGVMGENNTLVWKYADAGHVCHLCHPNCTYGPGNES |
| | LKAMLFCLFKLSSCNQSNDGSVSHQSGSPAAQESCLGWIPSLLPSE |
| | FQLGWGGCSHLHAWPSASVIITASSCH |
| | (SEQ ID NO: 162) |
| 4 | MRPSGTAGAALLALLAALCPASRALEEKKVCQGTSNKLTQLGTFE |
| (Uniprot ID No.: | DHFLSLQRMFNNCEVVLGNLEITYVQRNYDLSFLKTIQEVAGYVL |
| P00533-4) | IALNTVERIPLENLQIIRGNMYYENSYALAVLSNYDANKTGLKELP |
| | MRNLQEILHGAVRFSNNPALCNVESIQWRDIVSSDFLSNMSMDFQ |
| | NHLGSCQKCDPSCPNGSCWGAGEENCQKLTKIICAQQCSGRCRG |
| | KSPSDCCHNQCAAGCTGPRESDCLVCRKFRDEATCKDTCPPLML |
| | YNPTTYQMDVNPEGKYSFGATCVKKCPRNYVVTDHGSCVRACG |
| | ADSYEMEEDGVRKCKKCEGPCRKVCNGIGIGEFKDSLSINATNIK |
| | HFKNCTSISGDLHILPVAFRGDSFTHTPPLDPQELDILKTVKEITGFL |
| | LIQAWPENRTDLHAFENLEIIRGRTKQHGQFSLAVVSLNITSLGLRS |
| | LKEISDGDVIISGNKNLCYANTINWKKLFGTSGQKTKIISNRGENSC |
| | KATGQVCHALCSPEGCWGPEPRDCVSCRNVSRGRECVDKCNLLE |
| | GEPREFVENSECIQCHPECLPQAMNITCTGRGPDNCIQCAHYIDGP |
| | HCVKTCPAGVMGENNTLVWKYADAGHVCHLCHPNCTYGS |
| | (SEQ ID NO: 163) |

In some embodiments, novel antigen-binding sites that can bind to EGFR can be identified by screening for binding to the amino acid sequence defined by SEQ ID NOs: 160-163, a variant thereof, a mature extracellular fragment thereof or a fragment containing a domain of EGFR.

In each of the foregoing embodiments, it is contemplated herein that the scFv, VH and/or VL sequences that bind EGFR may contain amino acid alterations (e.g., at least 1, 2, 3, 4, 5, or 10 amino acid substitutions, deletions, or additions) in the framework regions of the VH and/or VL, where the alterations do not affect their ability to bind EGFR. For example, it is contemplated herein that scFv, VH and/or VL sequences that bind EGFR may contain cysteine heterodimerization mutations, facilitating formation of a disulfide bridge between the VH and VL of the scFv.

In certain embodiments, the second antigen-binding site of the multispecific binding protein disclosed herein binds human EGFR or the extracellular region thereof at a K_{D} value less than or equal to (affinity greater than or equal to) 10 nM, 9 nM, 8 nM, 7 nM, 6 nM, 5 nM, or 4 nM. In certain embodiments, the antigen-binding site of the present application binds human EGFR or the extracellular region thereof at a K_{D} value less than or equal to (affinity greater than or equal to) 4 nM. In certain embodiments, an antigen-binding site of the present application binds human EGFR or the extracellular region thereof at a K_{D} value less than or equal to (affinity greater than or equal to) about 2.0 nM, 2.1 nM, 2.2 nM, 2.3 nM, 2.4 nM, 2.5 nM, 2.6 nM, 2.7 nM, 2.8 nM, 2.9 nM, 3.0 nM, 3.1 nM, 3.2 nM, 3.3 nM, 3.4 nM, 3.5 nM, 3.6 nM, 3.7 nM, 3.8 nM, 3.9 nM, 4.0 nM, 4.1 nM, 4.2 nM, 4.3 nM, 4.4 nM, 4.5 nM, 4.6 nM, 4.7 nM, 4.8 nM, 4.9 nM or 5.0 nM. In certain embodiments, an antigen-binding site of the present application binds human EGFR or the extracellular region thereof at a K_{D} value in the range of about 1.0-3.5 nM, 1.0-4.0 nM, 1.0-4.5 nM, 1.0-5.0 nM, 1.5-3.5 nM, 1.5-4.0 nM, 1.5-4.5 nM, 1.5-5.0 nM, 2.0-3.5 nM, 2.0-4.0 nM, 2.0-4.5 nM, 2.0-5.0 nM, 2.5-3.5 nM, 2.5-4.0 nM, 2.5-4.5 nM, 2.5-5.0 nM, 3.0-3.5 nM, 3.0-4.0 nM, 3.0-4.5 nM, or 3.0-5.0 nM.

In some embodiments, a multi-specific binding protein described in the present disclosure binds to human EGFR, or the extracellular region thereof, with a K_{D} of 1.0 nM to 10 nM, 1.5 nM to 10 nM, 2.0 nM to 10 nM, 2.5 nM to 10 nM, 3.0 nM to 10 nM, 3.5 nM to 10 nM, 4.0 nM to 10 nM, 4.5 nM to 10 nM, 5.0 nM to 10 nM, 5.5 nM to 10 nM, 6.0 nM to 10 nM, 6.5 nM to 10 nM, 7.0 nM to 10 nM, 7.5 nM to 10.0 nM, 8.0 nM to 10 nM, 8.5 nM to 10 nM, 9.0 nM to 10 nM, 9.5 nM to 10 nM, 1.0 nM to 9.0 nM, 1.0 nM to 8.0 nM, 1.0 nM to 7.0 nM, 1.0 nM to 6.0 nM, 1.0 nM to 5.0 nM, 1.0 nM to 4.0 nM, 1.0 nM to 3.0 nM, 1.0 nM to 2.0 nM, 4.5 nM to 8.0 nM, 5.0 nM to 8.0 nM, 5.5 nM to 8.0 nM, 6.0 nM to 8.0 nM, 6.5 nM to 8.0 nM, 7.0 nM to 8.0 nM, 7.5 nM to 8.0 nM, 1.5 nM to 6.0 nM, 2.0 nM to 6.0 nM, 2.5 nM to 6.0 nM, 3.0 nM to 6.0 nM, 3.5 nM to 6.0 nM, 4.0 nM to 6.0 nM, 4.5 nM to 6.0 nM, 5.0 nM to 6.0 nM, 5.5 nM to 6.0 nM, 1.0 nM to 4.0 nM, 1.5 nM to 4.0 nM, 2.0 nM to 4.0 nM, 2.5 nM to 4.0 nM, 3.0 nM to 4.0 nM, 3.5 nM to 4.0 nM, or 1.5 nM to 2.0 nM, as measured by surface plasmon resonance (SPR). In some embodiments, a multi-specific binding protein described in the present disclosure binds to human EGFR with a K_{D} of 4.2 nM to 6.0 nM, 4.4 nM to 6.0 nM, 4.6 nM to 6.0 nM, 4.8 nM to 6.0 nM, 5.2 nM to 6.0 nM, 5.4 nM to 6.0 nM, 5.6 nM to 6.0 nM, 5.8 nM to 6.0 nM, 4.2 nM to 5.8 nM, 4.4 nM to 5.8 nM, 4.6 nM to 5.8 nM, 4.8 nM to 5.8 nM, 5.2 nM to 5.8 nM. 5.4 nM to 5.8 nM, 5.6 nM to 5.8 nM, 4.2 nM to 5.6 nM, 4.4 nM to 5.6 nM, 4.6 nM to 5.6 nM, 4.8 nM to 5.6 nM, 5.2 nM to 5.6 nM, 5.4 nM to 5.6 nM, 4.2 nM to 5.4 nM, 4.4 nM to 5.4 nM, 4.6 nM to 5.4 nM, 4.8 nM to 5.4 nM, 5.2 nM to 5.4 nM, 4.2 nM to 5.2 nM, 4.4 nM to 5.2 nM, 4.6 nM to 5.2 nM, 4.8 nM to 5.2 nM, 4.2 nM to 5.0 nM, 4.4 nM to 5.0 nM, 4.6 nM to 5.0 nM, or 4.8 nM to 5.0 nM. In some embodiments, a multi-specific binding protein described in the present disclosure binds to human EGFR with a K_{D} of 4.2 nM to 5.2 nM. In some embodiments, a multi-specific binding protein described in the present disclosure binds to human EGFR with a K_{D} of about 4.7 nM.

In some embodiments, a multi-specific binding protein described in the present disclosure binds to human EGFR with an association rate constant of 1.0×10⁵ l/Ms to 10.0×10⁵ l/Ms, 1.5×10⁵ l/Ms to 10.0×10⁵ l/Ms, 2.0×10⁵ l/Ms to 10.0×10⁵ l/Ms, 2.5×10⁵ l/Ms to 10.0×10⁵ l/Ms, 3.0×10⁵ l/Ms to 10.0×10⁵ l/Ms, 3.5×10⁵ l/Ms to 10.0×10⁵ l/Ms, 4.0×10⁵ l/Ms to 10.0×10⁵ l/Ms, 4.5×10⁵ l/Ms to 10.0×10⁵ l/Ms, 5.0×10⁵ l/Ms to 10.0×10⁵ l/Ms, 5.5×10⁵ l/Ms to 10.0×10⁵ l/Ms, 6.0×10⁵ l/Ms to 10.0×10⁵ l/Ms, 6.5×10⁵ l/Ms to 10.0×10⁵ l/Ms, 7.0×10⁵ l/Ms to 10.0×10⁵ l/Ms, 7.5×10⁵ l/Ms to 10.0×10⁵ l/Ms, 8.0×10⁵ 1/Ms to 10.0×10⁵ l/Ms, 8.5×10⁵ l/Ms to 10.0×10⁵ l/Ms, 9.0×10⁵ l/Ms to 10.0×10⁵ l/Ms, 9.5×10⁵ l/Ms to 10.0×10⁵ l/Ms, 1.0×10⁵ l/Ms to 9.0×10⁵ l/Ms, 1.0×10⁵ l/Ms to 8.0×10⁵ l/Ms, 1.0×10⁵ l/Ms to 7.0×10⁵ l/Ms, 1.0×10⁵ l/Ms to 6.0×10⁵ l/Ms, 1.0×10⁵ l/Ms to 5.0×10⁵ l/Ms, 1.0×10⁵ l/Ms to 4.0×10⁵ l/Ms, 1.0×10⁵ l/Ms to 3.0×10⁵ l/Ms, 1.0×10⁵ l/Ms to 2.0×10⁵ l/Ms, 5.0×10⁵ l/Ms to 8.0×10⁵ l/Ms 5.5×10⁵ l/Ms to 8.0×10⁵ l/Ms, 6.0×10⁵ 1/Ms to 8.0×10⁵ l/Ms, 6.5×10⁵ l/Ms to 8.0×10⁵ l/Ms, 7.0×10⁵ l/Ms to 8.0×10⁵ l/Ms, 7.5×10⁵ l/Ms to 8.0×10⁵ l/Ms, 1.5×10⁵ l/Ms to 6.0×10⁵ l/Ms, 2.0×10⁵ l/Ms to 6.0×10⁵ l/Ms, 2.5×10⁵ l/Ms to 6.0×10⁵ l/Ms, 3.0×10⁵ l/Ms to 6.0×10⁵ l/Ms, 3.5×10⁵ l/Ms to 6.0×10⁵ l/Ms, 4.0×10⁵ l/Ms to 6.0×10⁵ l/Ms, 4.5×10⁵ l/Ms to 6.0×10⁵ l/Ms, 5.0×10⁵ l/Ms to 6.0×10⁵ l/Ms, 5.5×10⁵ l/Ms to 6.0×10⁵ l/Ms, 1.5×10⁵ l/Ms to 4.0×10⁵ l/Ms, 2.0×10⁵ l/Ms to 4.0×10⁵ l/Ms, 2.5×10⁵ l/Ms to 4.0×10⁵ l/Ms, 3.0×10⁵ l/Ms to 4.0×10⁵ l/Ms, 3.5×10⁵ 1/Ms to 4.0×10⁵ l/Ms, or 1.5×10⁵ l/Ms to 2.0×10⁵ l/Ms, as measured by SPR. In some embodiments, a multi-specific binding protein described in the present disclosure binds to human EGFR with an association rate constant of 1.25×10⁵ l/Ms to 3.0×10⁵ l/Ms, 1.5×10⁵ l/Ms to 3.0×10⁵ l/Ms, 1.75×10⁵ l/Ms to 3.0×10⁵ l/Ms, 2.25×10⁵ l/Ms to 3.0×10⁵ l/Ms, 2.5×10⁵ 1/Ms to 3.0×10⁵ l/Ms, 2.75×10⁵ l/Ms to 3.0×10⁵ l/Ms, 1.25×10⁵ l/Ms to 2.5×10⁵ l/Ms, 1.5×10⁵ l/Ms to 2.5×10⁵ l/Ms, 1.75×10⁵ l/Ms to 2.5×10⁵ l/Ms, 2.25×10⁵ l/Ms to 2.5×10⁵ l/Ms, 1.25×10⁵ l/Ms to 2.25×10⁵ l/Ms, 1.5×10⁵ l/Ms to 2.25×10⁵ l/Ms, 1.75×10⁵ l/Ms to 2.25×10⁵ l/Ms, 1.25×10⁵ l/Ms to 2.0×10⁵ l/Ms, 1.5×10⁵ l/Ms to 2.0×10⁵ l/Ms, or 1.75×10⁵ l/Ms to 2.0×10⁵ l/Ms, as measured by SPR. In some embodiments, a multi-specific binding protein described in the present disclosure binds to human EGFR with an association rate constant of 1.5×10⁵ l/Ms to 2.5×10⁵ l/Ms. In some embodiments, a multi-specific binding protein described in the present disclosure binds to human EGFR with an association rate constant of about 2.0×10⁵ l/Ms, as measured by SPR.

In some embodiments, a multi-specific binding protein described in the present disclosure binds to human EGFR with a dissociation rate constant of 1.0×10⁻⁴ l/s to 15.0×10⁻⁴ l/s, 1.5×10⁻⁴ l/s to 15.0×10⁻⁴ l/s, 2.0×10⁻⁴ l/s to 15.0×10⁻⁴ l/s, 2.5×10⁻⁴ l/s to 15.0×10⁻⁴ l/s, 3.0×10⁻⁴ l/s to 15.0×10⁻⁴ l/s, 3.5×10⁻⁴ l/s to 15.0×10⁻⁴ l/s, 4.0×10⁻⁴ l/s to 15.0×10⁻⁴ l/s, 4.5×10⁻⁴ l/s to 15.0×10⁻⁴ l/s, 5.0×10⁻⁴ l/s to 15.0×10⁻⁴ l/s, 5.5×10⁻⁴ l/s to 15.0×10⁻⁴ l/s, 6.0×10⁻⁴ l/s to 15.0×10⁻⁴ l/s, 6.5×10⁻⁴ l/s to 15.0×10⁻⁴ l/s, 7.0×10⁻⁴ l/s to 15.0×10⁻⁴ l/s, 7.5×10⁻⁴ l/s to 15.0×10⁻⁴ l/s, 8.0×10⁻⁴ l/s to 15.0×10⁻⁴ l/s, 10.0×10⁻⁴ l/s to 15.0×10⁻⁴ l/s, 10.5×10⁻⁴ l/s to 15.0×10⁻⁴ l/s, 11.0×10⁻⁴ l/s to 15.0×10⁻⁴ k/s, 11.5×10⁻⁴ l/s to 15.0×10⁻⁴ l/s, 12.0×10⁻⁴ l/s to 15.0×10⁻⁴ l/s, 12.5×10⁻⁴ l/s to 15.0×10⁻⁴ l/s, 13.0×10⁻⁴ l/s to 15.0×10⁻⁴ l/s, 13.5×10⁻⁴ l/s to 15.0×10⁻⁴ l/s, 14.0×10⁻⁴ l/s to 15.0×10⁻⁴ l/s, 14.5×10⁻⁴ l/s to 15.0×10⁻⁴ l/s, 1.0×10⁻⁴ l/s to 14.0×10⁻⁴ l/s, 1.0×10⁻⁴ l/s to 13.0×10⁻⁴ l/s, 1.0×10⁻⁴ l/s to 12.0×10⁻⁴ l/s, 1.0×10⁻⁴ l/s to 11.0×10⁻⁴ l/s, 1.0×10⁻⁴ l/s to 10.0×10⁻⁴ l/s, 1.0×10⁻⁴ l/s to 9.0×10⁻⁴ l/s, 1.0×10⁻⁴ l/s to 8.0×10⁻⁴ l/s, 1.0×10⁻⁴ l/s to 7.0×10⁻⁴ l/s, 1.0×10⁻⁴ l/s to 6.0×10⁻⁴ l/s, 1.0×10⁻⁴ l/s to 5.0×10⁻⁴ l/s, 1.0×10⁻⁴ l/s to 4.0×10⁻⁴ l/s, 1.0×10⁻⁴ l/s to 3.0×10⁻⁴ l/s, 1.0×10⁻⁴ l/s to 2.0×10⁻⁴ l/s, 5.0×10⁻⁴ l/s to 10.0×10⁻⁴ l/s, 5.5×10⁻⁴ l/s to 10.0×10⁻⁴ l/s, 6.0×10⁻⁴ l/s to 10.0×10⁻⁴ l/s, 6.5×10⁻⁴ l/s to 10.0×10⁻⁴ l/s, 7.0×10⁻⁴ l/s to 10.0×10⁻⁴ l/s, 7.5×10⁻⁴ l/s to 10.0×10⁻⁴ l/s, 8.0×10⁻⁴ l/s to 10.0×10⁻⁴, 8.5×10⁻⁴ l/s to 10.0×10⁻⁴, 9.0×10⁻⁴ l/s to 10.0×10⁻⁴, 9.5×10⁻⁴ l/s to 10.0×10⁻⁴, l/s, 1.5×10⁻⁴ l/s to 5.0×10⁻⁴ l/s, 2.0×10⁻⁴ l/s to 5.0×10⁻⁴ l/s, 2.5×10⁻⁴ l/s to 5.0×10⁻⁴ l/s, 3.0×10⁻⁴ l/s to 5.0×10⁻⁴ l/s, 3.5×10⁻⁴ l/s to 5.0×10⁻⁴ l/s, 4.0×10⁻⁴ l/s to 5.0×10⁻⁴ l/s, or 4.5×10⁻⁴ l/s to 5.0×10⁻⁴ l/s, as measured by SPR. In some embodiments, a multi-specific binding protein described in the present disclosure binds to human EGFR with a dissociation rate constant of 9.0×10⁻⁴ l/s to 10.0×10⁻⁴ l/s, as measured by SPR. In some embodiments, a multi-specific binding protein described in the present disclosure binds to human EGFR with a dissociation rate constant of about 9.5×10⁻⁴ l/s, as measured by SPR.

In certain embodiments, the second antigen-binding site of the multi-specific binding protein disclosed herein has greater thermostability than a corresponding antigen-binding site having the VH and VL sequences of SEQ ID NOs: 135 and 139, where the second antigen-binding site does not include a G44C mutation in the VH and a G100C mutation in the VL. In certain embodiments, the second antigen-binding site of the multi-specific binding protein disclosed herein has greater thermostability than a corresponding antigen-binding site having an amino acid sequence of SEQ ID NO: 143 or 144, where the second antigen-binding site takes an scFv format in the VL-VH or VH-VL orientation, respectively. Methods of measuring thermostability include but are not limited to differential scanning calorimetry (DSC). In certain embodiments, where the second antigen-binding site takes an scFv format in the VL-VH orientation, a melting temperature of the second antigen-binding site (e.g., Tonset or Tm1 of a TriNKET in the F3′ format, as measured by DSC) is higher than the corresponding melting temperature of an scFv having the amino acid sequence of SEQ ID NO:143 by at least 1° C., 2° C., 3° C., 4° C., 5° C., or 6° C. In certain embodiments, where the second antigen-binding site takes an scFv format in the VH-VL orientation, a melting temperature of the second antigen-binding site (e.g., Tonset or Tm1 of a TriNKET in the F3′ format, as measured by DSC) is higher than the corresponding melting temperature of an scFv having the amino acid sequence of SEQ ID NO:144 by at least 1° C., 2° C., 3° C., 4° C., 5° C., or 6° C.

### Fc Domain

Within the Fc domain, CD16 binding is mediated by the hinge region and the CH2 domain. For example, within human IgG1, the interaction with CD16 is primarily focused on amino acid residues Asp 265-Glu 269, Asn 297-Thr 299, Ala 327-Ile 332, Leu 234-Ser 239, and carbohydrate residue N-acetyl-D-glucosamine in the CH2 domain (see, Sondermann et al., Nature, 406 (6793):267-273). Based on the known domains, mutations can be selected to enhance or reduce the binding affinity to CD16, such as by using phage-displayed libraries or yeast surface-displayed cDNA libraries, or can be designed based on the known three-dimensional structure of the interaction. Accordingly, in certain embodiments, the antibody Fc domain or the portion thereof includes a hinge and a CH2 domain. In certain embodiments, the antibody Fc domain, or the portion thereof, includes a hinge and a CH2 domain of a human IgG1 antibody.

In some embodiments, the antibody Fc domain of the multi-specific binding protein includes a first antibody Fc polypeptide and a second antibody Fc polypeptide. In some embodiments, the first antibody Fc polypeptide is linked to a Fab that binds NKG2D, and the second antibody Fc polypeptide is linked to an scFv that binds EGFR. In some embodiments, the first antibody Fc polypeptide is linked to the heavy chain portion of the Fab. In some embodiments, the scFv that binds EGFR is linked to the second antibody Fc polypeptide via a hinge including Ala-Ser or Gly-Ser.

The assembly of heterodimeric antibody heavy chains can be accomplished by expressing two different antibody heavy chain sequences in the same cell, which may lead to the assembly of homodimers of each antibody heavy chain as well as assembly of heterodimers. Promoting the preferential assembly of heterodimers can be accomplished by incorporating different mutations in the CH3 domain of each antibody heavy chain constant region as shown in U.S. Ser. No. 13/494,870, U.S. Ser. No. 16/028,850, U.S. Ser. No. 11/533,709, U.S. Ser. No. 12/875,015, U.S. Ser. No. 13/289,934, U.S. Ser. No. 14/773,418, U.S. Ser. No. 12/811,207, U.S. Ser. No. 13/866,756, U.S. Ser. No. 14/647,480, and U.S. Ser. No. 14/830,336. For example, mutations can be made in the CH3 domain based on human IgG1 and incorporating distinct pairs of amino acid substitutions within a first polypeptide and a second polypeptide that allow these two chains to selectively heterodimerize with each other. The positions of amino acid substitutions illustrated below are all numbered according to the EU index as in Kabat.

In one scenario, an amino acid substitution in the first polypeptide replaces the original amino acid with a larger amino acid, selected from arginine (R), phenylalanine (F), tyrosine (Y) or tryptophan (W), and at least one amino acid substitution in the second polypeptide replaces the original amino acid(s) with a smaller amino acid(s), chosen from alanine (A), serine (S), threonine (T), or valine (V), such that the larger amino acid substitution (a protuberance) fits into the surface of the smaller amino acid substitutions (a cavity). For example, one polypeptide can incorporate a T366W substitution, and the other can incorporate three substitutions including T366S, L368A, and Y407V.

An antibody heavy chain variable domain of the present application can optionally be coupled to an amino acid sequence at least 90% identical to an antibody constant region, such as an IgG constant region including hinge, CH2 and CH3 domains with or without a CH1 domain. In some embodiments, the amino acid sequence of the constant region is at least 90% identical to a human antibody constant region, such as a human IgG1 constant region, an IgG2 constant region, IgG3 constant region, or IgG4 constant region. In one embodiment, the antibody Fc domain or a portion thereof sufficient to bind CD16 includes an amino acid sequence at least 90% (e.g., at least 91%, at least 92%, at least 93%, at least 94%, at least 95%, at least 96%, at least 97%, at least 98%, at least 99%, or 100%) identical to wild-type human IgG1 Fc sequence: DKTHTCPPCPAPELLGGPSVFLFPPKPKDTLMISRTPEVTCVVVDVSHEDPEVKFNWYV DGVEVHNAKTKPREEQYNSTYRVVSVLTVLHQDWLNGKEYKCKVSNKALPAPIEKTIS KAKGQPREPQVYTLPPSRDELTKNQVSLTCLVKGFYPSDIAVEWESNGQPENNYKTTPP VLDSDGSFFLYSKLTVDKSRWQQGNVFSCSVMHEALHNHYTQKSLSLSPG (SEQ ID NO:118). In some other embodiments, the amino acid sequence of the constant region is at least 90% identical to an antibody constant region from another mammal, such as rabbit, dog, cat, mouse, or horse.

In some embodiments, the antibody constant domain linked to the scFv or the Fab fragment is able to bind to CD16. In some embodiments, the protein incorporates a portion of an antibody Fc domain (for example, a portion of an antibody Fc domain sufficient to bind CD16) that includes a hinge and a CH2 domain (for example, a hinge and a CH2 domain of a human IgG1 antibody), and/or amino acid sequences at least 90% (e.g., at least 90%, at least 91%, at least 92%, at least 93%, at least 94%, at least 95%, at least 96%, at least 97%, at least 98%, at least 99%, or 100%) identical to amino acid sequence 234-332 of a human IgG1 antibody, numbered according to the EU index.

One or more mutations can be incorporated into the constant region as compared to a human IgG1 constant region, for example at Q347, Y349, L351, S354, E356, E357, K360, Q362, S364, T366, L368, K370, N390, K392, T394, D399, S400, D401, F405, Y407, K409, T411 and/or K439. Exemplary substitutions include, for example, Q347E, Q347R, Y349S, Y349K, Y349T, Y349D, Y349E, Y349C, T350V, L351K, L351D, L351Y, S354C, E356K, E357Q, E357L, E357W, K360E, K360W, Q362E, S364K, S364E, S364H, S364D, T366V, T366I, T366L, T366M, T366K, T366W, T366S, L368E, L368A, L368D, K370S, N390D, N390E, K392L, K392M, K392V, K392F, K392D, K392E, T394F, T394W, D399R, D399K, D399V, S400K, S400R, D401K, F405A, F405T, Y407A, Y407I, Y407V, K409F, K409W, K409D, K409R, T411D, T411E, K439D, and K439E.

In certain embodiments, mutations that can be incorporated into the CH1 of a human IgG1 constant region may be at amino acid V125, F126, P127, T135, T139, A140, F170, P171, and/or V173. In certain embodiments, mutations that can be incorporated into the Cκ of a human IgG1 constant region may be at amino acid E123, F116, S176, V163, S174, and/or T164.

Alternatively, amino acid substitutions could be selected from the following sets of substitutions shown in Table 4.

TABLE 4
Exemplary Fc substitutions that Promote Heterodimerization

| | First Polypeptide | Second Polypeptide |
|---|---|---|
| Set 1 | S364E/F405A | Y349K/T394F |
| Set 2 | S364H/D401K | Y349T/T411E |
| Set 3 | S364H/T394F | Y349T/F405A |
| Set 4 | S364E/T394F | Y349K/F405A |
| Set 5 | S364E/T411E | Y349K/D401K |
| Set 6 | S364D/T394F | Y349K/F405A |
| Set 7 | S364H/F405A | Y349T/T394F |
| Set 8 | S364K/E357Q | L368D/K370S |
| Set 9 | L368D/K370S | S364K |
| Set 10 | L368E/K370S | S364K |
| Set 11 | K360E/Q362E | D401K |
| Set 12 | L368D/K370S | S364K/E357L |
| Set 13 | K370S | S364K/E357Q |
| Set 14 | F405L | K409R |
| Set 15 | K409R | F405L |

Alternatively, amino acid substitutions could be selected from the following sets of substitutions shown in Table 5.

TABLE 5
Exemplary Fc substitutions that Promote Heterodimerization

| | First Polypeptide | Second Polypeptide |
|---|---|---|
| Set 1 | K409W | D399V/F405T |
| Set 2 | Y349S | E357W |
| Set 3 | K360E | Q347R |
| Set 4 | K360E/K409W | Q347R/D399V/F405T |
| Set 5 | Q347E/K360E/K409W | Q347R/D399V/F405T |
| Set 6 | Y349S/K409W | E357W/D399V/F405T |

Alternatively, amino acid substitutions could be selected from the following sets of substitutions shown in Table 6.

TABLE 6
Exemplary Fc substitutions that Promote Heterodimerization

| | First Polypeptide | Second Polypeptide |
|---|---|---|
| Set 1 | T366K/L351K | L351D/L368E |
| Set 2 | T366K/L351K | L351D/Y349E |
| Set 3 | T366K/L351K | L351D/Y349D |
| Set 4 | T366K/L351K | L351D/Y349E/L368E |
| Set 5 | T366K/L351K | L351D/Y349D/L368E |
| Set 6 | E356K/D399K | K392D/K409D |

Alternatively, at least one amino acid substitution in each polypeptide chain could be selected from Table 7.

TABLE 7
Exemplary Fc substitutions that Promote Heterodimerization

| First Polypeptide | Second Polypeptide |
|---|---|
| L351Y, D399R, D399K, | T366V, T366I, T366L, T366M, |
| S400K, S400R, Y407A, | N390D, N390E, K392L, |
| Y407I, Y407V | K392M, K392V, K392F |
| | K392D, K392E, K409F, |
| | K409W, T411D and T411E |

Alternatively, at least one amino acid substitution could be selected from the following sets of substitutions in Table 8, where the position(s) indicated in the First Polypeptide column is replaced by any known negatively-charged amino acid, and the position(s) indicated in the Second Polypeptide Column is replaced by any known positively-charged amino acid.

TABLE 8
Exemplary Fc Positions for Substitutions

| First Polypeptide | Second Polypeptide |
|---|---|
| K392, K370, K409, or K439 | D399, E356, or E357 |

Alternatively, at least one amino acid substitution could be selected from the following set in Table 9, where the position(s) indicated in the First Polypeptide column is replaced by any known positively-charged amino acid, and the position(s) indicated in the Second Polypeptide Column is replaced by any known negatively-charged amino acid.

TABLE 9
Exemplary Fc Positions for Substitutions

| First Polypeptide | Second Polypeptide |
|---|---|
| D399, E356, or E357 | K409, K439, K370, or K392 |

Alternatively, amino acid substitutions could be selected from the following sets in Table 10.

TABLE 10
Exemplary Fc substitutions that
Promote Heterodimerization

| First Polypeptide | Second Polypeptide |
|---|---|
| T350V, L351Y, | T350V, T366L, |
| F405A, and Y407V | K392L, and T394W |

Alternatively, or in addition, the structural stability of a hetero-multimeric protein may be increased by introducing S354C on either of the first or second polypeptide chain, and Y349C on the opposing polypeptide chain, which forms an artificial disulfide bridge within the interface of the two polypeptides.

In some embodiments, the amino acid sequence of one polypeptide chain of the antibody constant region differs from the amino acid sequence of an IgG1 constant region at position T366, and where the amino acid sequence of the other polypeptide chain of the antibody constant region differs from the amino acid sequence of an IgG1 constant region at one or more positions selected from the group consisting of T366, L368 and Y407.

In some embodiments, the amino acid sequence of one polypeptide chain of the antibody constant region differs from the amino acid sequence of an IgG1 constant region at one or more positions selected from the group consisting of T366, L368 and Y407, and the amino acid sequence of the other polypeptide chain of the antibody constant region differs from the amino acid sequence of an IgG1 constant region at position T366.

In some embodiments, the amino acid sequence of one polypeptide chain of the antibody constant region differs from the amino acid sequence of an IgG1 constant region at one or more positions selected from the group consisting of E357, K360, Q362, S364, L368, K370, T394, D401, F405, and T411 and the amino acid sequence of the other polypeptide chain of the antibody constant region differs from the amino acid sequence of an IgG1 constant region at one or more positions selected from the group consisting of Y349, E357, S364, L368, K370, T394, D401, F405 and T411.

In some embodiments, the amino acid sequence of one polypeptide chain of the antibody constant region differs from the amino acid sequence of an IgG1 constant region at one or more positions selected from the group consisting of Y349, E357, S364, L368, K370, T394, D401, F405 and T411 and the amino acid sequence of the other polypeptide chain of the antibody constant region differs from the amino acid sequence of an IgG1 constant region at one or more positions selected from the group consisting of E357, K360, Q362, S364, L368, K370, T394, D401, F405, and T411.

In some embodiments, the amino acid sequence of one polypeptide chain of the antibody constant region differs from the amino acid sequence of an IgG1 constant region at one or more positions selected from the group consisting of L351, D399, S400 and Y407 and the amino acid sequence of the other polypeptide chain of the antibody constant region differs from the amino acid sequence of an IgG1 constant region at one or more positions selected from the group consisting of T366, N390, K392, K409 and T411.

In some embodiments, the amino acid sequence of one polypeptide chain of the antibody constant region differs from the amino acid sequence of an IgG1 constant region at one or more positions selected from the group consisting of T366, N390, K392, K409 and T411 and the amino acid sequence of the other polypeptide chain of the antibody constant region differs from the amino acid sequence of an IgG1 constant region at one or more positions selected from the group consisting of L351, D399, S400 and Y407.

In some embodiments, the amino acid sequence of one polypeptide chain of the antibody constant region differs from the amino acid sequence of an IgG1 constant region at one or more positions selected from the group consisting of Q347, Y349, K360, and K409, and the amino acid sequence of the other polypeptide chain of the antibody constant region differs from the amino acid sequence of an IgG1 constant region at one or more positions selected from the group consisting of Q347, E357, D399 and F405.

In some embodiments, the amino acid sequence of one polypeptide chain of the antibody constant region differs from the amino acid sequence of an IgG1 constant region at one or more positions selected from the group consisting of Q347, E357, D399 and F405, and the amino acid sequence of the other polypeptide chain of the antibody constant region differs from the amino acid sequence of an IgG1 constant region at one or more positions selected from the group consisting of Y349, K360, Q347 and K409.

In some embodiments, the amino acid sequence of one polypeptide chain of the antibody constant region differs from the amino acid sequence of an IgG1 constant region at one or more positions selected from the group consisting of K370, K392, K409 and K439, and the amino acid sequence of the other polypeptide chain of the antibody constant region differs from the amino acid sequence of an IgG1 constant region at one or more positions selected from the group consisting of D356, E357 and D399.

In some embodiments, the amino acid sequence of one polypeptide chain of the antibody constant region differs from the amino acid sequence of an IgG1 constant region at one or more positions selected from the group consisting of D356, E357 and D399, and the amino acid sequence of the other polypeptide chain of the antibody constant region differs from the amino acid sequence of an IgG1 constant region at one or more positions selected from the group consisting of K370, K392, K409 and K439.

In some embodiments, the amino acid sequence of one polypeptide chain of the antibody constant region differs from the amino acid sequence of an IgG1 constant region at one or more positions selected from the group consisting of L351, E356, T366 and D399, and the amino acid sequence of the other polypeptide chain of the antibody constant region differs from the amino acid sequence of an IgG1 constant region at one or more positions selected from the group consisting of Y349, L351, L368, K392 and K409.

In some embodiments, the amino acid sequence of one polypeptide chain of the antibody constant region differs from the amino acid sequence of an IgG1 constant region at one or more positions selected from the group consisting of Y349, L351, L368, K392 and K409, and the amino acid sequence of the other polypeptide chain of the antibody constant region differs from the amino acid sequence of an IgG1 constant region at one or more positions selected from the group consisting of L351, E356, T366 and D399.

In some embodiments, the amino acid sequence of one polypeptide chain of the antibody constant region differs from the amino acid sequence of an IgG1 constant region by an S354C substitution and the amino acid sequence of the other polypeptide chain of the antibody constant region differs from the amino acid sequence of an IgG1 constant region by a Y349C substitution.

In some embodiments, the amino acid sequence of one polypeptide chain of the antibody constant region differs from the amino acid sequence of an IgG1 constant region by a Y349C substitution and the amino acid sequence of the other polypeptide chain of the antibody constant region differs from the amino acid sequence of an IgG1 constant region by an S354C substitution.

In some embodiments, the first antibody Fc polypeptide is a human IgG1 Fc polypeptide incorporating K360E and K409W substitutions, numbered according to the EU index. In some embodiments, the second antibody Fc polypeptide is a human IgG1 Fc polypeptide incorporates Q347R, D399V, and F405T substitutions, numbered according to the EU index. In some embodiments, the antibody Fc domain includes a first antibody Fc polypeptide linked to the Fab and a second antibody Fc polypeptide linked to the scFv, where the first antibody Fc polypeptide is a human IgG1 Fc polypeptide incorporating K360E and K409W substitutions, and the second antibody Fc polypeptide is a human IgG1 Fc polypeptide incorporating Q347R, D399V, and F405T substitutions, numbered according to the EU index.

In some embodiments, the first antibody Fc polypeptide is a human IgG1 Fc polypeptide incorporating Q347R, D399V, and F405T substitutions, numbered according to the EU index. In some embodiments, the antibody Fc domain includes a first antibody Fc polypeptide linked to the Fab and a second antibody Fc polypeptide linked to the scFv; the first antibody Fc polypeptide is a human IgG1 Fc polypeptide incorporating Q347R, D399V, and F405T substitutions; and the second antibody Fc polypeptide is a human IgG1 Fc polypeptide incorporating K360E and K409W substitutions, numbered according to the EU index. In some embodiments, the second antibody Fc polypeptide is a human IgG1 Fc polypeptide incorporating K360E and K409W substitutions, numbered according to the EU index. In some embodiments, the amino acid sequence of one polypeptide chain of the antibody constant region differs from the amino acid sequence of an IgG1 constant region by a T366W substitution and the amino acid sequence of the other polypeptide chain of the antibody constant region differs from the amino acid sequence of an IgG1 constant region by T366S, T368A, and Y407V substitutions.

In some embodiments, the amino acid sequence of one polypeptide chain of the antibody constant region differs from the amino acid sequence of an IgG1 constant region by T366S, T368A, and Y407V substitutions and the amino acid sequence of the other polypeptide chain of the antibody constant region differs from the amino acid sequence of an IgG1 constant region by a T366W substitution.

In some embodiments, the amino acid sequence of one polypeptide chain of the antibody constant region differs from the amino acid sequence of an IgG1 constant region by T350V, L351Y, F405A, and Y407V substitutions and the amino acid sequence of the other polypeptide chain of the antibody constant region differs from the amino acid sequence of an IgG1 constant region by T350V, T366L, K392L, and T394W substitutions.

In some embodiments, the amino acid sequence of one polypeptide chain of the antibody constant region differs from the amino acid sequence of an IgG1 constant region by T350V, T366L, K392L, and T394W substitutions and the amino acid sequence of the other polypeptide chain of the antibody constant region differs from the amino acid sequence of an IgG1 constant region by T350V, L351Y, F405A, and Y407V substitutions.

In some embodiments, a multi-specific binding protein described in the present disclosure binds to human CD16, with a binding affinity (K_{D}) of 10 nM to 200 nM, 20 nM to 200 nM, 30 nM to 200 nM, 40 nM to 200 nM, 50 nM to 200 nM, 60 nM to 200 nM, 70 nM to 200 nM, 80 nM to 200 nM, 90 nM to 200 nM, 100 nM to 200 nM, 110 nM to 200 nM, 120 nM to 200 nM, 130 nM to 200 nM, 140 nM to 200 nM, 150 nM to 200 nM, 160 nM to 200 nM, 170 nM to 200 nM, 180 nM to 200 nM, 190 nM to 200 nM, 10 nM to 190 nM, 10 nM to 180 nM, 10 nM to 170 nM, 10 nM to 160 nM, 10 nM to 150 nM, 10 nM to 140 nM, 10 nM to 130 nM, 10 nM to 120 nM, 10 nM to 110 nM, 10 nM to 100 nM, 10 nM to 90 nM, 10 nM to 80 nM, 10 nM to 70 nM, 10 nM to 60 nM, 10 nM to 50 nM, 10 nM to 40 nM, 10 nM to 30 nM, 10 nM to 20 nM, 20 nM to 160 nM, 30 nM to 160 nM, 40 nM to 160 nM, 50 nM to 160 nM, 60 nM to 160 nM, 70 nM to 160 nM, 80 nM to 160 nM, 90 nM to 160 nM, 100 nM to 160 nM, 110 nM to 160 nM, 120 nM to 160 nM, 130 nM to 160 nM, 140 nM to 160 nM, 150 nM to 160 nM, 20 nM to 140 nM, 30 nM to 140 nM, 40 nM to 140 nM, 50 nM to 140 nM, 60 nM to 140 nM, 70 nM to 140 nM, 80 nM to 140 nM, 90 nM to 140 nM, 100 nM to 140 nM, 110 nM to 140 nM, 120 nM to 140 nM, 130 nM to 140 nM, 20 nM to 120 nM, 30 nM to 120 nM, 40 nM to 120 nM, 50 nM to 120 nM, 60 nM to 120 nM, 70 nM to 120 nM, 80 nM to 120 nM, 90 nM to 120 nM, 100 nM to 120 nM, 110 nM to 120 nM, 20 nM to 100 nM, 30 nM to 100 nM, 40 nM to 100 nM, 50 nM to 100 nM, 60 nM to 100 nM, 70 nM to 100 nM, 80 nM to 100 nM, or 90 nM to 100 nM, as measured by SPR. In some embodiments, a multi-specific binding protein described in the present disclosure binds to human CD16, with a binding affinity (K_{D}) of 40 nM to 165 nM, 42 nM to 165 nM, 44 nM to 165 nM, 46 nM to 165 nM, 48 nM to 165 nM, 50 nM to 165 nM, 42 nM to 160 nM, 44 nM to 160 nM, 46 nM to 160 nM, 48 nM to 160 nM, 40 nM to 155 nM, 42 nM to 155 nM, 44 nM to 155 nM, 46 nM to 155 nM, 48 nM to 155 nM, or 50 nM to 165 nM, as measured by SPR. In some embodiments, a multi-specific binding protein described in the present disclosure binds to human CD16, with a binding affinity (K_{D}) of 48 nM to 160 nM, as measured by SPR. In some embodiments, a multi-specific binding protein described in the present disclosure binds to human CD16, with a binding affinity (K_{D}) of about 81 nM, as measured by SPR.

### Exemplary Multi-Specific Binding Proteins

Listed below are examples of TriNKETs incorporating an antigen-binding site that binds EGFR and an antigen-binding site that binds NKG2D each linked to an antibody Fc polypeptide; each of the antibody Fc polypeptides include mutations that enable heterodimerization of the two antibody Fc polypeptides.

TriNKETs are contemplated in the F3 format, i.e., where the antigen-binding site that binds EGFR is a Fab, and the antigen-binding site that binds NKG2D is an scFv. All the TriNKETs shown infra are in the F3′ format, i.e., the antigen-binding site that binds EGFR is an scFv and the antigen-binding site that binds NKG2D is a Fab. In each TriNKET, the scFv may include substitution of Cys in the VH and VL regions (e.g., C44 and C100 in the VH and VL, respectively), facilitating formation of a disulfide bridge between the VH and VL of the scFv.

The VH and VL of the scFv can be connected via a linker, e.g., a peptide linker. In certain embodiments, the peptide linker is a flexible linker. Regarding the amino acid composition of the linker, peptides are selected with properties that confer flexibility, do not interfere with the structure and function of the other domains of the proteins of the present application, and resist cleavage from proteases. For example, glycine and serine residues generally provide protease resistance. In certain embodiments, the VL is linked N-terminal or C-terminal to the VH via a (GlyGlyGlyGlySer)₄ ((G₄S)₄) linker (SEQ ID NO:119).

The length of the linker (e.g., flexible linker) can be “short,” e.g., 0, 1, 2, 3, 4, 5, 6, 7, 8, 9, 10, 11 or 12 amino acid residues, or “long,” e.g., at least 13 amino acid residues. In certain embodiments, the linker is 10-50, 10-40, 10-30, 10-25, 10-20, 15-50, 15-40, 15-30, 15-25, 15-20, 20-50, 20-40, 20-30, or 20-25 amino acid residues in length.

In certain embodiments, the linker includes or consists of a (GS)ₙ (SEQ ID NO:120), (GGS)ₙ (SEQ ID NO:121), (GGGS)ₙ (SEQ ID NO:122), (GGSG)ₙ (SEQ ID NO:123), (GGSGG)ₙ (SEQ ID NO:124), and (GGGGS)ₙ (SEQ ID NO: 125) sequence, wherein n is 1, 2, 3, 4, 5, 6, 7, 8, 9, 10, 11, 12, 13, 14, 15, 16, 17, 18, 19, or 20. In certain embodiments, the linker includes or consists of an amino acid sequence selected from SEQ ID NO:119, and 126-134, as listed in

TABLE 11

| SEQ ID | Amino Acid Sequence |
|---|---|
| SEQ ID | GSGSGSGSGSGSGSGSGSGS |
| NO: 126 | |
| SEQ ID | GGSGGSGGSGGSGGSGGSGGSGGSGGSGGS |
| NO: 127 | |
| SEQ ID | GGGSGGGSGGGSGGGSGGGSGGGSGGGSG |
| NO: 128 | GGSGGGSGGGS |
| SEQ ID | GGSGGGSGGGSGGGSGGGSGGGSGGGSGG |
| NO: 129 | GSGGGSGGGSG |
| SEQ ID | GGSGGGGSGGGGSGGGGSGGGGSGGGGSG |
| NO: 130 | GGGSGGGGSGGGGSGGGGSGG |
| SEQ ID | GGGGSGGGGSGGGGSGGGGSGGGGSGGGG |
| NO: 131 | SGGGGSGGGGSGGGGSGGGGS |
| SEQ ID | GGGGSGGGGSGGGGSGGGGS |
| NO: 119 | |
| SEQ ID | GGGGSGGGGSGGGGS |
| NO: 132 | |
| SEQ ID | GGGGSGGGGSGGGGSGGGGSGGGGSGGGG |
| NO: 133 | SGGGGSGGGGSGGGGSGGGGSGGGGSGGG |
| | GSGGGGSGGGGSGGGGSGGGGSGGGGSGG |
| | GGSGGGGSGGGGS |
| SEQ ID | GGSGGGGSGGGGSGGGGSGGGGSGGGGSG |
| NO: 134 | GGGSGGGGSGGGGSGGGGSGGGGSGGGGS |
| | GGGGSGGGGSGGGGSGGGGSGGGGSGGGG |
| | SGGGGSGGGGSGG |

In the F3′-TriNKETs, an EGFR-binding scFv is linked to the N-terminus of an antibody Fc polypeptide via a Gly-Ser linker. The Ala-Ser or Gly-Ser linker is included at the elbow hinge region sequence to balance between flexibility and optimal geometry. In certain embodiments, an additional sequence Thr-Lys-Gly can be added N-terminal or C-terminal to the Ala-Ser or Gly-Ser sequence at the hinge.

As used herein to describe these exemplary TriNKETs, an antibody Fc polypeptide includes an antibody hinge, CH2, and CH3. In each exemplary TriNKET, the antibody Fc polypeptide linked to an scFv incorporates the mutations of Q347R, D399V, and F405T, and the antibody Fc polypeptide linked to a Fab incorporates matching mutations K360E and K409W for forming a heterodimer. The antibody Fc polypeptide linked to the scFv further includes an S354C substitution in the CH3 domain, which forms a disulfide bond with a Y349C substitution on the antibody Fc polypeptide linked to the Fab. These substitutions are bold in the sequences described in this subsection.

In some embodiments, a multi-specific protein in the present disclosure includes: (i) a first antigen-binding site that binds NKG2D; (ii) a second antigen-binding site that binds EGFR; and (iii) an antibody Fc domain, or portion thereof sufficient to bind CD16, or a third antigen-binding site that binds CD16. In some embodiments, the first antigen-binding site is a Fab, and the second antigen-binding site is an scFv.

Some multi-specific proteins in the present disclosure incorporate: (i) a Fab with a VH and a VL that bind NKG2D; (ii) an scFv with a VH and a VL that bind EGFR; and (iii) an antibody Fc domain.

Some multi-specific proteins in the present disclosure incorporate: (i) a Fab with a VH and a VL that that bind NKG2D; (ii) an scFv that binds EGFR, where the scFv includes: 1) a VH having the CDR1, CDR2, and CDR3 sequences of SEQ ID NOs: 136, 157, and 138, respectively; and a VL having the CDR1, CDR2, and CDR3 sequences of SEQ ID NOs: 140, 141, and 151, respectively; or 2) a VH having the CDR1, CDR2, and CDR3 sequences of SEQ ID NOs: 136, 146, and 138, respectively; and a VL having the CDR1, CDR2, and CDR3 sequences of SEQ ID NOs: 140, 141, and 142, respectively; and (iii) an antibody Fc domain.

In some embodiments, the VL of the scFv is linked to the VH of the scFv via a flexible linker having a sequence selected from SEQ ID NO:119 or any one of SEQ ID NOs: 126-134. In some embodiments, the VL of the scFv is linked to the VH of the scFv via a flexible linker having the sequence of SEQ ID NO:119. In some embodiments, the VL of the scFv is positioned to the N-terminus of the VH of the scFv. In some embodiments, the VH of the scFv is positioned to the N-terminus of the VL of the scFv.

In some embodiments, the VH of the scFv forms a disulfide bridge with the VL of the scFv. In some embodiments, the disulfide bridge is formed between a cysteine residue (naturally occurring or introduced by mutation) at position 44 (C44) of the VH of the scFv and a cysteine residue (naturally occurring or introduced by mutation) at position 100 (C100) of the VL, numbered under the Kabat numbering scheme.

In some embodiments, the antibody Fc domain of the multi-specific binding proteins of the present disclosure includes a first antibody Fc polypeptide and a second antibody Fc polypeptide. In some embodiments, the first antibody Fc polypeptide is linked to the Fab and the second antibody Fc polypeptide is linked to the scFv. In some embodiments, the first antibody Fc polypeptide is linked to a heavy chain portion of the Fab. In some embodiments, the scFv is linked to the second antibody Fc polypeptide via a hinge including the amino acid sequence Ala-Ser or Gly-Ser. In some embodiments, the first and second antibody Fc polypeptides each include a hinge and a CH2 domain of a human IgG1 antibody. In some embodiments, the first and second antibody Fc polypeptides each include an amino acid sequence at least 90% identical to amino acids 234-332 of a wild-type human IgG1 antibody, numbered according to the EU index. In some embodiments, the first and second antibody Fc polypeptides each incorporate different mutations promoting heterodimerization. In some embodiments, the first antibody Fc polypeptide is a human IgG1 Fc polypeptide incorporating K360E and K409W substitutions, numbered according to the EU index. In some embodiments, the second antibody Fc polypeptide is a human IgG1 Fc polypeptide incorporating Q347R, D399V, and F405T substitutions, numbered according to the EU index.

In other embodiments, the first antibody Fc polypeptide is linked to the scFv and the second antibody Fc polypeptide is linked to the Fab. In some embodiments, the second antibody Fc polypeptide is linked to a heavy chain portion of the Fab. In some embodiments, the scFv is linked to the first antibody Fc polypeptide via a hinge including Ala-Ser or Gly-Ser. In some embodiments, the first and second antibody Fc polypeptides each include a hinge and a CH2 domain of a human IgG1 antibody. In some embodiments, the first and second antibody Fc polypeptides each include an amino acid sequence at least 90% identical to amino acids 234-332 of a wild-type human IgG1 antibody, numbered according to the EU index. In some embodiments, the first and second antibody Fc polypeptides each incorporate different mutations promoting heterodimerization. In some embodiments, the first antibody Fc polypeptide is a human IgG1 Fc polypeptide incorporating K360E and K409W substitutions, numbered according to the EU index. In some embodiments, the second antibody Fc polypeptide is a human IgG1 Fc polypeptide incorporating Q347R, D399V, and F405T substitutions, numbered according to the EU index.

In some embodiments, the Fab includes: (a) a VH having the CDR1, CDR2, and CDR3 sequences of the VH CDR sequences of an antibody disclosed in Table 1; and (b) a VL having the CDR1, CDR2, and CDR3 sequences of the VL CDR sequences of an antibody disclosed in Table 1. In some embodiments, the Fab includes: (a) a VH having the CDR1, CDR2, and CDR3 sequences of SEQ ID NOs: 80 or 81, 82, and 111 or 112, respectively; and (b) a VL having the CDR1, CDR2, and CDR3 sequences of SEQ ID NOs: 86, 77, and 87, respectively. In some embodiments, the Fab includes a VH with an amino acid sequence at least 90% (e.g., at least 90%, at least 91%, at least 92%, at least 93%, at least 94%, at least 95%, at least 96%, at least 97%, at least 98%, at least 99%, or at least 100%) identical to SEQ ID NO:110, and a VL with an amino acid sequence at least 90% (e.g., at least 90%, at least 91%, at least 92%, at least 93%, at least 94%, at least 95%, at least 96%, at least 97%, at least 98%, at least 99%, or at least 100%) identical to SEQ ID NO:85. In some embodiments, the Fab includes a VH with the amino acid sequence of SEQ ID NO:110, and a VL with the amino acid sequence of SEQ ID NO:85.

In some embodiments, the Fab includes (a) a VH having the CDR1, CDR2, and CDR3 sequences of SEQ ID NOs: 80 or 81, 82, and 96 or 97, respectively; and (b) a VL having the CDR1, CDR2, and CDR3 sequences of SEQ ID NOs: 86, 77, and 87, respectively. In some embodiments, the Fab includes a VH with an amino acid sequence at least 90% (e.g., at least 90%, at least 91%, at least 92%, at least 93%, at least 94%, at least 95%, at least 96%, at least 97%, at least 98%, at least 99%, or at least 100%) identical to SEQ ID NO:95, and a VL with an amino acid sequence at least 90% (e.g., at least 90%, at least 91%, at least 92%, at least 93%, at least 94%, at least 95%, at least 96%, at least 97%, at least 98%, at least 99%, or at least 100%) identical to SEQ ID NO:85. In some embodiments, the Fab includes a VH with the amino acid sequence of SEQ ID NO:95 and a VL with the amino acid sequence of SEQ ID NO:85.

In some embodiments, the scFv includes (a) a VH having the CDR1, CDR2, and CDR3 sequences of SEQ ID NOs: 136, 157, and 138, respectively, and a VL having the CDR1, CDR2, and CDR3 sequences of SEQ ID NOs: 140, 141, and 151, respectively; or (b) a VH having the CDR1, CDR2, and CDR3 sequences of SEQ ID NOs: 136, 146, and 138, respectively, and a VL having the CDR1, CDR2, and CDR3 sequences of SEQ ID NOs: 140, 141, and 142, respectively. In some embodiments, the scFv includes: a VH having the CDR1, CDR2, and CDR3 sequences of SEQ ID NOs: 136, 157, and 138, respectively, and a VL having the CDR1, CDR2, and CDR3 sequences of SEQ ID NOs: 140, 141, and 151, respectively. In some embodiments, the scFv includes (a) a VH with an amino acid sequence at least 90% identical (e.g., at least 90%, at least 91%, at least 92%, at least 93%, at least 94%, at least 95%, at least 96%, at least 97%, at least 98%, at least 99%, or at least 100%) to SEQ ID NO:156 and (b) a VL with an amino acid sequence at least 90% identical (e.g., at least 90%, at least 91%, at least 92%, at least 93%, at least 94%, at least 95%, at least 96%, at least 97%, at least 98%, at least 99%, or at least 100%) to SEQ ID NO:150. In some embodiments, the scFv has the amino acid sequence of SEQ ID NO:158.

In some embodiments, the scFv includes: a VH having the CDR1, CDR2, and CDR3 sequences of SEQ ID NOs: 136, 146, and 138, respectively, and a VL having the CDR1, CDR2, and CDR3 sequences of SEQ ID NOs: 140, 141, and 151, respectively. In some embodiments, the scFv includes (a) a VH with an amino acid sequence at least 90% identical (e.g., at least 90%, at least 91%, at least 92%, at least 93%, at least 94%, at least 95%, at least 96%, at least 97%, at least 98%, at least 99%, or at least 100%) to SEQ ID NO:170 and (b) a VL with an amino acid sequence at least 90% identical (e.g., at least 90%, at least 91%, at least 92%, at least 93%, at least 94%, at least 95%, at least 96%, at least 97%, at least 98%, at least 99%, or at least 100%) to SEQ ID NO:171. In some embodiments, the scFv includes (a) a VH with the amino acid of SEQ ID NO:170 and (b) a VL with the amino acid sequence of SEQ ID NO:171. In some embodiments, the scFv has the amino acid sequence of SEQ ID NO:152.

In some embodiments, the scFv includes: a VH having the CDR1, CDR2, and CDR3 sequences of SEQ ID NOs: 136, 137, and 138, respectively, and a VL having the CDR1, CDR2, and CDR3 sequences of SEQ ID NOs: 140, 141, and 151, respectively. In some embodiments, the scFv includes (a) a VH with an amino acid sequence at least 90% identical (e.g., at least 90%, at least 91%, at least 92%, at least 93%, at least 94%, at least 95%, at least 96%, at least 97%, at least 98%, at least 99%, or at least 100%) to SEQ ID NO:135 and (b) a VL with an amino acid sequence at least 90% identical (e.g., at least 90%, at least 91%, at least 92%, at least 93%, at least 94%, at least 95%, at least 96%, at least 97%, at least 98%, at least 99%, or at least 100%) to SEQ ID NO:150. In some embodiments, the scFv includes (a) a VH with the amino acid of SEQ ID NO:135 and (b) a VL with the amino acid sequence of SEQ ID NO:150. In some embodiments, the scFv has the amino acid sequence of SEQ ID NO:154.

In some embodiments, the scFv includes: a VH having the CDR1, CDR2, and CDR3 sequences of SEQ ID NOs: 136, 146, and 138, respectively, and a VL having the CDR1, CDR2, and CDR3 sequences of SEQ ID NOs: 140, 141, and 142, respectively. In some embodiments, the scFv includes (a) a VH with an amino acid sequence at least 90% identical (e.g., at least 90%, at least 91%, at least 92%, at least 93%, at least 94%, at least 95%, at least 96%, at least 97%, at least 98%, at least 99%, or at least 100%) to SEQ ID NO:145 and (b) a VL with an amino acid sequence at least 90% identical (e.g., at least 90%, at least 91%, at least 92%, at least 93%, at least 94%, at least 95%, at least 96%, at least 97%, at least 98%, at least 99%, or at least 100%) to SEQ ID NO:147. In some embodiments, the scFv includes (a) a VH with amino acid of SEQ ID NO:145 and (b) a VL with the amino acid sequence of SEQ ID NO:147. In some embodiments, the scFv has the amino acid sequence of SEQ ID NO:148.

In some embodiments, the scFv includes: a VH having the CDR1, CDR2, and CDR3 sequences of SEQ ID NOs: 136, 137, and 138, respectively, and a VL having the CDR1, CDR2, and CDR3 sequences of SEQ ID NOs: 140, 141, and 142, respectively. In some embodiments, the scFv includes (a) a VH with an amino acid sequence at least 90% identical (e.g., at least 90%, at least 91%, at least 92%, at least 93%, at least 94%, at least 95%, at least 96%, at least 97%, at least 98%, at least 99%, or at least 100%) to SEQ ID NO:135 and (b) a VL with an amino acid sequence at least 90% identical (e.g., at least 90%, at least 91%, at least 92%, at least 93%, at least 94%, at least 95%, at least 96%, at least 97%, at least 98%, at least 99%, or at least 100%) to SEQ ID NO:139. In some embodiments, the scFv includes (a) a VH with the amino acid of SEQ ID NO:135 and (b) a VL with the amino acid sequence of SEQ ID NO:139. In some embodiments, the scFv has the amino acid sequence of SEQ ID NO:143.

For example, one TriNKET described in the present disclosure is EGFR-TriNKET-1. EGFR-TriNKET-1 includes (a) an EGFR-scFv-1 (VL-VH) sequence provided in Table 2, in the orientation of VH positioned C-terminal to VL, linked to an Fc domain polypeptide and (b) an NKG2D-binding Fab fragment derived from A49MI including a heavy chain portion including a VH and a CH1 domain, and a light chain portion including a VL and a CL, where the CH1 domain is connected to the Fc domain polypeptide. EGFR-TriNKET-1 includes three polypeptides: EGFR-scFv-1 (VL-VH)-Fc (SEQ ID NO:172), A49MI-VH-CH1-Fc (SEQ ID NO:164), and A49MI-VL-CL (SEQ ID NO:165).

EGFR-scFv-1 (VL-VH)-Fc. Residues in bold indicate
mutated residues in the Fc domain.
(SEQ ID NO: 172)
DIQMTQSPSSLSASVGDRVTITCQASQDISNYLNWYQQKPGKAPKLLIYDASNLETGVPS
RFSGSGSGTDFTFTISSLQPEDIATYFCQHFDHLPLAFGCGTKVEIKGGGGSGGGGSGGG
GSGGGGSQVQLQESGPGLVKPSETLSLTCTVSGGSVSSGDYYWTWIRQSPGKCLEWIGH
IYYSGNTNYNPSLKSRLTISIDTSKTQFSLKLSSVTAADTAIYYCVRDRVTGAFDIWGQGT
MVTVSS
GS
DKTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEKTIS
KAKGQPREPRVYTLPPCRDELTKNQVSLTCLVKGFYPSDIAVEWESNGQPENNYKTTPP
VLVSDGSFTLYSKLTVDKSRWQQGNVFSCSVMHEALHNHYTQKSLSLSPG
A49MI-VH-CH1-Fc. Residues in bold indicate mutated residues in
the Fc domain and underlined sequences indicate CDR sequences.
(SEQ ID NO: 164)
EVQLVESGGGLVKPGGSLRLSCAASGFTFSSYSMNWVRQAPGKGLEWVSSISSSSSYIY
YADSVKGRFTISRDNAKNSLYLQMNSLRAEDTAVYYCARGAPIGAAAGWFDPWGQGT
LVTVSS
ASTKGPSVFPLAPSSKSTSGGTAALGCLVKDYFPEPVTVSWNSGALTSGVHTFPAVLQSS
GLYSLSSVVTVPSSSLGTQTYICNVNHKPSNTKVDKKVEPKSCDKTHTCPPCPAPELLGG
PSVFLFPPKPKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREPQVCTLPPSR
DELTENQVSLTCLVKGFYPSDIAVEWESNGQPENNYKTTPPVLDSDGSFFLYSWLTVDK
SRWQQGNVFSCSVMHEALHNHYTQKSLSLSPG
A49MI-VL-CL. Underlined sequences indicate CDR sequences.
(SEQ ID NO: 165)
DIQMTQSPSSVSASVGDRVTITCRASQGISSWLAWYQQKPGKAPKLLIYAASSLQSGVPS
RFSGSGSGTDFTLTISSLQPEDFATYYCQQGVSFPRTFGGGTKVEIK
RTVAAPSVFIFPPSDEQLKSGTASVVCLLNNFYPREAKVQWKVDNALQSGNSQESVTEQ
DSKDSTYSLSSTLTLSKADYEKHKVYACEVTHQGLSSPVTKSFNRGEC

EGFR-scFv-1 (VL-VH)-Fc (SEQ ID NO:166) represents the full sequence of an EGFR-binding scFv linked to an Fc domain polypeptide via a hinge including Gly-Ser. The Fc domain polypeptide linked to the scFv includes Q347R, D399V, and F405T substitutions for heterodimerization and an S354C substitution for forming a disulfide bond with a Y349C substitution in A49MI-VH-CH1-Fc as described below. The scFv includes a heavy chain variable domain of EGFR-binder-1 in Table 2 connected to the C-terminus of a light chain variable domain of EGFR-binder-1 in Table 2 via a (G₄S)₄ linker (SEQ ID NO: 119); the scFv includes substitutions of Cys in the VH and VL regions at G44 and S100, respectively, thereby facilitating formation of a disulfide bridge between the VH and VL of the scFv.

A49MI-VH-CH1-Fc (SEQ ID NO:164) represents the heavy chain portion of the Fab fragment, which incorporates a heavy chain variable domain of NKG2D-binding A49MI (SEQ ID NO:95) and a CH1 domain, connected to an Fc domain. The Fc domain polypeptide in A49MI-VH-CH1-Fc includes a Y349C substitution in the CH3 domain, which forms a disulfide bond with an S354C substitution on the Fc polypeptide in EGFR-scFv-2 (VL-VH)-Fc. In A49MI-VH-CH1-Fc, the Fc domain also includes K360E and K409W substitutions for heterodimerization with the Fc in EGFR-scFv-2 (VL-VH)-Fc.

A49MI-VL-CL (SEQ ID NO:165) represents the light chain portion of the Fab fragment including a light chain variable domain of NKG2D-binding A49MI (SEQ ID NO:85) and a light chain constant domain.

Some multi-specific binding proteins of the present disclosure incorporate:

(a) a Fab including a VH with the CDR1, CDR2, and CDR3 sequences of SEQ ID NOs: 81, 82, and 97, respectively; and a VL with the CDR1, CDR2, and CDR3 sequences of SEQ ID NOs: 86, 77, and 87, respectively;

(b) an scFv including a VH with the CDR1, CDR2, and CDR3 sequences of SEQ ID NOs: 136, 137, and 138, respectively; and a VL with the CDR1, CDR2, and CDR3 sequences of SEQ ID NOs: 140, 141, and 142, respectively; and

(c) an antibody Fe domain including a first antibody Fc polypeptide linked to the Fab and a second antibody Fc polypeptide linked to the scFv; the first antibody Fc polypeptide is a human IgG1 Fc polypeptide incorporating K360E and K409W substitutions, and the second antibody Fc polypeptide is a human IgG1 Fc polypeptide incorporating Q347R, D399V, and F405T substitutions, numbered according to the EU index.

Some multi-specific binding proteins of the present disclosure incorporate:

(a) a Fab including a VH with the amino acid sequence of SEQ ID NO:95 and a VL with the amino acid sequence of SEQ ID NO:85;

(b) an scFv including a VH with the amino acid sequence of SEQ ID NO:135 and a VL of the scFv with the amino acid sequence of SEQ ID NO:139; and

(c) an antibody Fc domain including a first antibody Fc polypeptide linked to the Fab and a second antibody Fc polypeptide linked to the scFv; the first antibody Fc polypeptide is a human IgG1 Fc polypeptide incorporating K360E and K409W substitutions and the second antibody Fc polypeptide is a human IgG1 Fc polypeptide incorporating Q347R, D399V, and F405T substitutions, numbered according to the EU index.

Some multi-specific binding proteins of the present disclosure incorporate:

(a) a Fab including a VH with the amino acid sequence of SEQ ID NO:95 and a VL with the amino acid sequence of SEQ ID NO:85;

(b) an scFv with the amino acid sequence of SEQ ID NO:143; and

(c) an antibody Fc domain including a first antibody Fc polypeptide linked to the Fab and a second antibody Fc polypeptide linked to the scFv; the first antibody Fc polypeptide is a human IgG1 Fc polypeptide incorporating K360E and K409W substitutions and the second antibody Fc polypeptide is a human IgG1 Fc polypeptide incorporating Q347R, D399V, and F405T substitutions, numbered according to the EU index.

Another TriNKET described in the present disclosure is EGFR-TriNKET-2. EGFR-TriNKET-2 includes (a) an EGFR-scFv-2 (VL-VH) sequence provided in Table 2, in the orientation of VH positioned C-terminal to VL, linked to an Fc domain polypeptide and (b) an NKG2D-binding Fab fragment derived from A49MI including a heavy chain portion including a VH and a CH1 domain, and a light chain portion including a VL and a CL, where the CH1 domain is connected to the Fc domain polypeptide. EGFR-TriNKET-2 includes three polypeptides: EGFR-scFv-2 (VL-VH)-Fc (SEQ ID NO:166), A49MI-VH-CH1-Fc (SEQ ID NO:164), and A49MI-VL-CL (SEQ ID NO:165).

EGFR-scFv-2 (VL-VH)-Fc. Residues in bold indicate mutated residues
in the Fc domain.
(SEQ ID NO: 166)
DIQMTQSPSSLSASVGDRVTITCQASQDISNYLNWYQQKPGKAPKLLIYDASNLETGVPS
RFSGSGSGTDFTFTISSLQPEDIATYYCQHFDHLPLAFGCGTKVEIK
GGGGSGGGGSGGGGSGGGGS
QVQLQESGPGLVKPSETLSLTCTVSGGSVSSGDYYWTWIRQSPGKCLEWIGHIYYSGNT
NYNPRLKSRLTISIDTSKTQFSLKLSSVTAADTAIYYCVRDRVTGAFDIWGQGTMVTVSS
GS
DKTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEKTIS
KAKGQPREPRVYTLPPCRDELTKNQVSLTCLVKGFYPSDIAVEWESNGQPENNYKTTPP
VLVSDGSFTLYSKLTVDKSRWQQGNVFSCSVMHEALHNHYTQKSLSLSPG
A49MI-VH-CH1-Fc. Residues in bold indicate mutated residues in
the Fc domain and underlined sequences indicate CDR sequences.
(SEQ ID NO: 164)
EVQLVESGGGLVKPGGSLRLSCAASGFTESSYSMNWVRQAPGKGLEWVSSISSSSSYIY
YADSVKGRFTISRDNAKNSLYLQMNSLRAEDTAVYYCARGAPIGAAAGWFDPWGQGT
LVTVSS
ASTKGPSVFPLAPSSKSTSGGTAALGCLVKDYFPEPVTVSWNSGALTSGVHTFPAVLQSS
GLYSLSSVVTVPSSSLGTQTYICNVNHKPSNTKVDKKVEPKSCDKTHTCPPCPAPELLGG
PSVFLFPPKPKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREPQVCTLPPSR
DELTENQVSLTCLVKGFYPSDIAVEWESNGQPENNYKTTPPVLDSDGSFFLYSWLTVDK
SRWQQGNVFSCSVMHEALHNHYTQKSLSLSPG
A49MI-VL-CL. Underlined sequences indicate CDR sequences.
(SEQ ID NO: 165)
DIQMTQSPSSVSASVGDRVTITCRASQGISSWLAWYQQKPGKAPKLLIYAASSLQSGVPS
RFSGSGSGTDFTLTISSLQPEDFATYYCQQGVSFPRTFGGGTKVEIK
RTVAAPSVFIFPPSDEQLKSGTASVVCLLNNFYPREAKVQWKVDNALQSGNSQESVTEQ
DSKDSTYSLSSTLTLSKADYEKHKVYACEVTHQGLSSPVTKSFNRGEC

EGFR-scFv-2 (VL-VH)-Fc (SEQ ID NO:166) represents the full sequence of an EGFR-binding scFv linked to an Fc domain polypeptide via a hinge including Gly-Ser. The Fc domain polypeptide linked to the scFv includes Q347R, D399V, and F405T substitutions for heterodimerization and an S354C substitution for forming a disulfide bond with a Y349C substitution in A49MI-VH-CH1-Fc as described below. The scFv includes a heavy chain variable domain of EGFR-binder-2 in Table 2 connected to the C-terminus of a light chain variable domain of EGFR-binder-2 in Table 2 via a (G₄S)₄ linker (SEQ ID NO: 119). The scFv also includes substitutions of Cys in the VH and VL regions at G44 and S100, respectively, thereby facilitating formation of a disulfide bridge between the VH and VL of the scFv.

Some multi-specific binding proteins of the present disclosure incorporate:

(a) a Fab including a VH with the CDR1, CDR2, and CDR3 sequences of SEQ ID NOs: 81, 82, and 97, respectively; and a VL with the CDR1, CDR2, and CDR3 sequences of SEQ ID NOs: 86, 77, and 87, respectively;

(b) an scFv including a VH with the CDR1, CDR2, and CDR3 sequences of SEQ ID NOs: 136, 146, and 138, respectively; and a VL with the CDR1, CDR2, and CDR3 sequences of SEQ ID NOs: 140, 141, and 142, respectively; and

(c) an antibody Fc domain including a first antibody Fc polypeptide linked to the Fab and a second antibody Fc polypeptide linked to the scFv; the first antibody Fc polypeptide is a human IgG1 Fc polypeptide incorporating K360E and K409W substitutions, and the second antibody Fc polypeptide is a human IgG1 Fc polypeptide incorporating Q347R, D399V, and F405T substitutions, numbered according to the EU index.

Some multi-specific binding proteins of the present disclosure incorporate:

(a) a Fab including a VH with the amino acid sequence of SEQ ID NO:95 and a VL with the amino acid sequence of SEQ ID NO:85;

(b) an scFv including a VH with the amino acid sequence of SEQ ID NO:145 and a VL with the amino acid sequence of SEQ ID NO:147; and

(c) an antibody Fc domain including a first antibody Fc polypeptide linked to the Fab and a second antibody Fc polypeptide linked to the scFv; the first antibody Fc polypeptide is a human IgG1 Fc polypeptide incorporating K360E and K409W substitutions, and the second antibody Fc polypeptide is a human IgG1 Fc polypeptide incorporating Q347R, D399V, and F405T substitutions, numbered according to the EU index.

Some multi-specific binding proteins of the present disclosure incorporate:

(a) a Fab including a VH with the amino acid sequence of SEQ ID NO:95 and a VL with the amino acid sequence of SEQ ID NO:85;

(b) an scFv with the amino acid sequence of SEQ ID NO:148; and

(c) an antibody Fe domain including a first antibody Fc polypeptide linked to the Fab and a second antibody Fc polypeptide linked to the scFv; the first antibody Fc polypeptide is a human IgG1 Fc polypeptide incorporating K360E and K409W substitutions, and the second antibody Fc polypeptide is a human IgG1 Fc polypeptide incorporating Q347R, D399V, and F405T substitutions, numbered according to the EU index.

Another TriNKET described in the present disclosure is EGFR-TriNKET-3. EGFR-TriNKET-3 includes (a) an EGFR-scFv-3 (VL-VH) sequence provided in Table 2, in the orientation of VH positioned C-terminal to VL, linked to an Fc domain polypeptide and (b) an NKG2D-binding Fab fragment derived from A49MI including a heavy chain portion having a heavy chain variable domain and a CH1 domain, and a light chain portion having a light chain variable domain and a light chain constant domain; the CH1 domain is connected to the Fc domain polypeptide. EGFR-TriNKET-3 includes three polypeptides: EGFR-scFv-3 (VL-VH)-Fc (SEQ ID NO:167), A49MI-VH-CH1-Fc (SEQ ID NO:164), and A49MI-VL-CL (SEQ ID NO:165).

EGFR-scFv-3 (VL-VH)-Fc. Residues in bold indicate mutated residues
in the Fc domain.
(SEQ ID NO: 167)
DIQMTQSPSSLSASVGDRVTITCQASQDISNYLNWYQQKPGKAPKLLIYDASNLETGVPS
RFSGSGSGTDFTFTISSLQPEDIATYYCQHFRHLPLAFGCGTKVEIK
GGGGSGGGGSGGGGSGGGGS
QVQLQESGPGLVKPSETLSLTCTVSGGSVSSGDYYWTWIRQSPGKCLEWIGHIYYSGNT
NYNPRLKSRLTISIDTSKTQFSLKLSSVTAADTAIYYCVRDRVTGAFDIWGQGTMVTVSS
GS
DKTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEKTIS
KAKGQPREPRVYTLPPCRDELTKNQVSLTCLVKGFYPSDIAVEWESNGQPENNYKTTPP
VLVSDGSFTLYSKLTVDKSRWQQGNVFSCSVMHEALHNHYTQKSLSLSPG

EGFR-scFv-3 (VL-VH)-Fc (SEQ ID NO:167) represents the full sequence of an EGFR-binding scFv linked to an Fc domain via a hinge including Gly-Ser. The Fc domain polypeptide linked to the scFv includes Q347R, D399V, and F405T substitutions for heterodimerization and an S354C substitution for forming a disulfide bond with a Y349C substitution in A49MI-VH-CH1-Fc as described below. The scFv includes a heavy chain variable domain of EGFR-binder-3 in Table 2 connected to the C-terminus of a light chain variable domain of EGFR-binder-3 clone in Table 2 via a (G₄S)₄ linker (SEQ ID NO: 119);the scFv also includes substitutions of Cys in the VH and VL regions at G44 and S100, respectively, thereby facilitating formation of a disulfide bridge between the VH and VL of the scFv.

Some multi-specific binding proteins of the present disclosure incorporate:

(a) a Fab including a VH with the CDR1, CDR2, and CDR3 sequences of SEQ ID NOs: 81, 82, and 97, respectively; and a VL with the CDR1, CDR2, and CDR3 sequences of SEQ ID NOs: 86, 77, and 87, respectively;

(b) an scFv including a VH with the CDR1, CDR2, and CDR3 sequences of SEQ ID NOs: 136, 146, and 138, respectively; and a VL with the CDR1, CDR2, and CDR3 sequences of SEQ ID NOs: 140, 141, and 151, respectively; and

(c) an antibody Fc domain including a first antibody Fc polypeptide linked to the Fab and a second antibody Fc polypeptide linked to the scFv; the first antibody Fc polypeptide is a human IgG1 Fc polypeptide incorporating K360E and K409W substitutions, and the second antibody Fc polypeptide is a human IgG1 Fc polypeptide incorporating Q347R, D399V, and F405T substitutions, numbered according to the EU index.

Some multi-specific binding proteins of the present disclosure incorporate:

(a) a Fab including a VH with the amino acid sequence of SEQ ID NO:95, and a VL with the amino acid sequence of SEQ ID NO:85;

(b) an scFv including a VH with the amino acid sequence of SEQ ID NO:170, and a VL with the amino acid sequence of SEQ ID NO:171; and

(c) an antibody Fc domain including a first antibody Fc polypeptide linked to the Fab and a second antibody Fc polypeptide linked to the scFv; the first antibody Fc polypeptide is a human IgG1 Fc polypeptide incorporating K360E and K409W substitutions, and the second antibody Fc polypeptide is a human IgG1 Fc polypeptide incorporating Q347R, D399V, and F405T substitutions, numbered according to the EU index.

Some multi-specific binding proteins of the present disclosure incorporate:

(a) a Fab including a VH with the amino acid sequence of SEQ ID NO:95, and a VL with the amino acid sequence of SEQ ID NO:85;

(b) an scFv including the amino acid sequence of SEQ ID NO:152; and

(c) an antibody Fc domain including a first antibody Fc polypeptide linked to the Fab and a second antibody Fc polypeptide linked to the scFv; the first antibody Fc polypeptide is a human IgG1 Fc polypeptide incorporating K360E and K409W substitutions, and the second antibody Fc polypeptide is a human IgG1 Fc polypeptide incorporating Q347R, D399V, and F405T substitutions, numbered according to the EU index.

In some embodiments, multi-specific binding proteins of the present disclosure incorporate:

(a) a first polypeptide with the amino acid sequence of SEQ ID NO:167;

(b) a second polypeptide with the amino acid sequence of SEQ ID NO:164; and

(c) a third polypeptide with the amino acid sequence of SEQ ID NO:165.

Another TriNKET described in the present disclosure is EGFR-TriNKET-4. EGFR-TriNKET-4 includes (a) an EGFR-scFv-4 (VL-VH) sequence provided in Table 2, in the orientation of VH positioned C-terminal to VL, linked to an Fc domain polypeptide and (b) an NKG2D-binding Fab fragment derived from A49MI including a heavy chain portion including a heavy chain variable domain and a CH1 domain, and a light chain portion including a light chain variable domain and a light chain constant domain; the CH1 domain is connected to the Fc domain polypeptide. EGFR-TriNKET-4 includes three polypeptides: EGFR-scFv-4 (VL-VH)-Fc (SEQ ID NO:168), A49MI-VH-CH1-Fc (SEQ ID NO:164), and A49MI-VL-CL (SEQ ID NO:165).

EGFR-scFv-4 (VL-VH)-Fc. Residues in bold indicate mutated residues
in the Fc domain.
(SEQ ID NO: 168)
DIQMTQSPSSLSASVGDRVTITCQASQDISNYLNWYQQKPGKAPKLLIYDASNLETGVPS
RFSGSGSGTDFTFTISSLQPEDIATYYCQHFRHLPLAFGCGTKVEIK
GGGGSGGGGSGGGGSGGGGS
QVQLQESGPGLVKPSETLSLTCTVSGGSVSSGDYYWTWIRQSPGKCLEWIGHIYYSGNT
NYNPSLKSRLTISIDTSKTQFSLKLSSVTAADTAIYYCVRDRVTGAFDIWGQGTMVTVSS
GS
DKTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEKTIS
KAKGQPREPRVYTLPPCRDELTKNQVSLTCLVKGFYPSDIAVEWESNGQPENNYKTTPP
VLVSDGSFTLYSKLTVDKSRWQQGNVFSCSVMHEALHNHYTQKSLSLSPG

EGFR-scFv-4 (VL-VH)-Fc (SEQ ID NO:168) represents the full sequence of an EGFR-binding scFv linked to an Fc domain polypeptide via a hinge including Gly-Ser. The Fc domain polypeptide linked to the scFv includes Q347R, D399V, and F405T substitutions for heterodimerization and an S354C substitution for forming a disulfide bond with a Y349C substitution in A49MI-VH-CH1-Fc as described below. The scFv includes a heavy chain variable domain of EGFR-binder-4 clone in Table 2 connected to the C-terminus of a light chain variable domain of EGFR-binder-4 clone in Table 2 via a (G₄S)₄ linker (SEQ ID NO: 119), where the scFv further includes substitution of Cys in the VH and VL regions at G44 and S100, respectively, thereby facilitating formation of a disulfide bridge between the VH and VL of the scFv.

Some multi-specific binding proteins of the present disclosure incorporate:

(a) a Fab including a VH with the CDR1, CDR2, and CDR3 sequences of SEQ ID NOs: 81, 82, and 97, respectively; and a VL with the CDR1, CDR2, and CDR3 sequences of SEQ ID NOs: 86, 77, and 87, respectively;

(b) an scFv including a VH with the CDR1, CDR2, and CDR3 sequences of SEQ ID NOs: 136, 137, and 138, respectively; and a VL with the CDR1, CDR2, and CDR3 sequences of SEQ ID NOs: 140, 141, and 151, respectively; and

(c) an antibody Fc domain including a first antibody Fc polypeptide linked to the Fab and a second antibody Fc polypeptide linked to the scFv; the first antibody Fc polypeptide is a human IgG1 Fc polypeptide incorporating K360E and K409W substitutions, and the second antibody Fc polypeptide is a human IgG1 Fc polypeptide incorporating Q347R, D399V, and F405T substitutions, numbered according to the EU index.

Some multi-specific binding proteins of the present disclosure incorporate:

(a) a Fab including a VH with the amino acid sequence of SEQ ID NO:95 and a VL with the amino acid sequence of SEQ ID NO:85;

(b) an scFv including a VH with the amino acid sequence of SEQ ID NO:135 and a VL with the amino acid sequence of SEQ ID NO:150; and

(c) an antibody Fc domain including a first antibody Fc polypeptide linked to the Fab and a second antibody Fc polypeptide linked to the scFv; the first antibody Fc polypeptide is a human IgG1 Fc polypeptide incorporating K360E and K409W substitutions, and the second antibody Fc polypeptide is a human IgG1 Fc polypeptide incorporating Q347R, D399V, and F405T substitutions, numbered according to the EU index.

Some multi-specific binding proteins of the present disclosure incorporate:

(a) a Fab including a VH with the amino acid sequence of SEQ ID NO:95 and a VL with the amino acid sequence of SEQ ID NO:85;

(b) an scFv with the amino acid sequence of SEQ ID NO:154; and

(c) an antibody Fc domain including a first antibody Fc polypeptide linked to the Fab and a second antibody Fc polypeptide linked to the scFv; the first antibody Fc polypeptide is a human IgG1 Fc polypeptide incorporating K360E and K409W substitutions, and the second antibody Fc polypeptide is a human IgG1 Fc polypeptide incorporating Q347R, D399V, and F405T substitutions, numbered according to the EU index.

In certain embodiments, a TriNKET described in the present disclosure is identical to one of the exemplary TriNKETs described above that includes the EW-RVT Fc mutations, except that the Fc domain polypeptide linked to the NKG2D-binding Fab fragment includes the substitutions of Q347R, D399V, and F405T, and the Fc domain polypeptide linked to EGFR binding scFv includes matching substitutions K360E and K409W for forming a heterodimer. In certain embodiments, a TriNKET described in the present disclosure is identical to one of the exemplary TriNKETs described above that includes the KiH Fc mutations, except that the Fc domain polypeptide linked to the NKG2D-binding Fab fragment incorporates the “hole” substitutions of T366S, L368A, and Y407V, and the Fc domain polypeptide linked to EGFR-binding scFv incorporates the “knob” substitution of T366W for forming a heterodimer.

A skilled person in the art would appreciate that during production and/or storage of proteins, N-terminal glutamate (E) or glutamine (Q) can be cyclized to form a lactam (e.g., spontaneously or catalyzed by an enzyme present during production and/or storage). Accordingly, in some embodiments where the N-terminal residue of an amino acid sequence of a polypeptide is E or Q, a corresponding amino acid sequence with the E or Q replaced with pyroglutamate is also contemplated herein.

A skilled person in the art would also appreciate that during protein production and/or storage, the C-terminal lysine (K) of a protein can be removed (e.g., spontaneously or catalyzed by an enzyme present during production and/or storage). Such removal of K is often observed with proteins that include an Fc domain at its C-terminus. Accordingly, in some embodiments where the C-terminal residue of an amino acid sequence of a polypeptide (e.g., an Fc domain sequence) is K, a corresponding amino acid sequence with the K removed is also contemplated herein.

The multi-specific binding proteins described above can be made using recombinant DNA technology well known to a skilled person in the art. For example, a first nucleic acid sequence encoding the first immunoglobulin heavy chain can be cloned into a first expression vector; a second nucleic acid sequence encoding the second immunoglobulin heavy chain can be cloned into a second expression vector; a third nucleic acid sequence encoding the immunoglobulin light chain can be cloned into a third expression vector; and the first, second, and third expression vectors can be stably transfected together into host cells to produce the multimeric proteins.

To achieve the highest yield of the multi-specific binding protein, different ratios of the first, second, and third expression vector can be explored to determine the optimal ratio for transfection into the host cells. After transfection, single clones can be isolated for cell bank generation using methods known in the art, such as limited dilution, ELISA, FACS, microscopy, or Clonepix.

Clones can be cultured under conditions suitable for bio-reactor scale-up and maintained expression of the multi-specific binding protein. The multi-specific binding proteins can be isolated and purified using methods known in the art including centrifugation, depth filtration, cell lysis, homogenization, freeze-thawing, affinity purification, gel filtration, ion exchange chromatography, hydrophobic interaction exchange chromatography, and mixed-mode chromatography.

### II. Characteristics of the Multi-Specific Binding Proteins

The multi-specific binding proteins described herein include an NKG2D-binding site, a tumor-associated antigen-binding site that binds EGFR, and an antibody Fc domain or a portion thereof sufficient to bind CD16, or an antigen-binding site that binds CD16. In some embodiments, the multi-specific binding proteins contains an additional antigen-binding site that binds to the same tumor-associated antigen (EGFR), as exemplified in the F4-TriNKET format (e.g., FIGS. 2C and 2D).

All approved EGFR antagonists are accompanied by significant toxicities, and tumors frequently become resistant, resulting in patient relapse. Multi-specific binding proteins of the present disclosure are engineered to bind to both EGFR and NKG2D, while retaining a fully functional human IgG1 Fc domain capable of also binding to Fc receptors, and have been designed to induce a greater anti-tumor response as compared to approved EGFR-targeting therapies, such as panitumumab and cetuximab. In some embodiments, multi-specific proteins of the present disclosure activate resting NK cells of all CD16a genotypes, resulting in more efficient degranulation, cytokine release, and potent NK-mediated lysis of tumor cells expressing a range of EGFR levels, as compared to approved EGFR-targeting therapies. In some embodiments, multi-specific binding proteins of the present disclosure induce higher levels of antibody-dependent phagocytosis of EGFR-expressing tumor cells by macrophages as compared to approved EGFR-targeting therapies.

EGFR-TriNKETs of the present disclosure are useful for use a therapy against EGFR-expressing cancers, including cancers with low levels of EGFR. The EGFR-TriNKETs of the present disclosure may bind monovalently and with high affinity to EGFR via an scFv domain that is derived from the sequence used in the approved EGFR-targeting antibody panitumumab. In some embodiments, targeting by EGFR-TriNKET to EGFR-expressing tumors results in anti-tumor activity via EGFR signal inhibition.

EGFR-TriNKETs of the present disclosure are capable of binding with low affinity to NKG2D, resulting in transient receptor binding that avoids stable binding of peripheral immune cell subsets, while allowing for strong functional agonism with CD16a when localized to tumor cells via EGFR engagement. In some embodiments, EGFR-TriNKETs of the present disclosure directly engage CD8⁺ T cells, which express NKG2D. EGFR-TriNKETs of the present disclosure possess a functional Fc domain that, like other wild-type IgG1s, can bind to Fc receptors, including CD16a on NK cells. Resting NK cells can be activated by CD16a. Once activated, NKG2D binding leads to additional signaling, resulting in strong NK cell-mediated cytotoxicity that results in greater potency and greater enhanced tumor cell lysis than that seen with mAbs. In addition, the Fc domain of EGFR-TriNKET can mediate antibody-dependent cellular phagocytosis. The IgG1 Fc domain can also interact with other Fc receptors, including neonatal Fc receptor (FcRn), which confers a long, antibody-like half-life.

In some embodiments, multi-specific binding proteins of the present disclosure stimulate NK cells through NKG2D and CD16a-activating receptors and induce killing of EGFR-expressing tumor cells. In some embodiments, multi-specific binding proteins of the present disclosure do not trigger NK-mediated lysis of non-malignant EGF-expressing cells. In some embodiments, multi-specific binding proteins of the present disclosure do not activate NK cells in the absence of EGFR-expressing tumor cells. In some embodiments, multi-specific binding proteins of the present disclosure do not induce cytokine release in the absence of EGFR-expressing tumor cells. In some embodiments, multi-specific binding proteins of the present disclosure induce the release of chemokines and/or cytokines including, but not limited to, IFNγ, TNFα, CCL4, CCL5, CXCL9, and CXCL10. In some embodiments, multi-specific binding proteins of the present disclosure promote recruitment of immune effector cells to EGFR-expressing tumors.

In some embodiments, multi-specific binding proteins of the present disclosure activate CD8⁺ T cells via NKG2D stimulation to directly kill EGFR-expressing human cancer cells. However, multi-specific binding proteins of the present disclosure preferably do not activate CD8⁺ T cells in the periphery, nor CD4⁺ T cells.

In some embodiments, multi-specific binding proteins of the present disclosure inhibit EGFR-dependent signaling to a greater degree than approved EGFR-targeting therapies, thereby inhibiting proliferation of cancer cells that is dependent on EGFR activation.

In some embodiments, multi-specific binding proteins of the present disclosure overcome acquired resistance and/or insensitivity to approved EGFR antagonist therapies. In some embodiments, multi-specific binding proteins of the present disclosure have a lower toxicity and a more manageable safety profile as compared to approved EGFR-targeting therapies.

In some embodiments, multi-specific binding proteins of the present disclosure bind to EGFR monovalently and with high affinity as compared to other anti-EGFR antibodies, thereby making them potent therapies against EGFR-expressing cancers, including those with low levels of EGFR. In some embodiments, multi-specific binding proteins of the present disclosure bind to NKG2D with low affinity, resulting in transient receptor binding that avoids stable binding to peripheral immune cell subsets, but allows for strong functional agonism with CD16a when localized to tumor cells via EGFR engagement. In some embodiments, multi-specific binding proteins of the present disclosure specifically bind human NKG2D but not cynomolgus monkey NKG2D.

In addition to activation of CD16a, multi-specific binding proteins of the present disclosure are also capable of interacting with other Fc receptors, including neonatal Fc receptor (FcRn), to confer a long, antibody-like systemic half-life.

### III. Therapeutic Applications

In some embodiments, provided herein are methods of treating an unresectable solid tumor in a subject by administering an effective amount of a multi-specific binding protein, or a pharmaceutical formulation thereof. The multi-specific binding protein includes: a first antigen-binding site that binds NKG2D; a second antigen-binding site that binds EGFR; and an antibody Fc domain or a portion thereof sufficient to bind CD16, or a third antigen-binding site that binds CD16.

In some embodiments, provided herein are methods of treating an unresectable solid tumor in a subject by administering an effective amount of a multi-specific binding protein, or a pharmaceutical formulation thereof. The multi-specific binding protein includes: a first antigen binding site that binds NKG2D; a second antigen binding site that binds EGFR incorporating (i) a VH having the CDR1, CDR2, and CDR3 sequences of SEQ ID NOs: 136, 157, and 138, respectively; and a VL having the CDR1, CDR2, and CDR3 sequences of SEQ ID NOs: 140, 141, and 151, respectively; (ii) a VH having the CDR1, CDR2, and CDR3 sequences of SEQ ID NOs: 136, 146, and 138, respectively; and a VL having the CDR1, CDR2, and CDR3 sequences of SEQ ID NOs: 140, 141, and 142, respectively; (iii) a VH having the CDR1, CDR2, and CDR3 sequences of SEQ ID NOs: 136, 146, and 138, respectively; and a VL having the CDR1, CDR2, and CDR3 sequences of SEQ ID NOs: 140, 141, and 151, respectively; (iv) a VH having the CDR1, CDR2, and CDR3 sequences of SEQ ID NOs: 136, 137, and 138, respectively; and a VL having the CDR1, CDR2, and CDR3 sequences of SEQ ID NOs: 140, 141, and 151, respectively; or (v) a VH having the CDR1, CDR2, and CDR3 sequences of SEQ ID NOs: 136, 137, and 138, respectively; and a VL having the CDR1, CDR2, and CDR3 sequences of SEQ ID NOs: 140, 141, and 142, respectively; and an antibody Fc domain or a portion thereof sufficient to bind CD16, or a third antigen-binding site that binds CD16.

In some embodiments, provided herein are methods of treating an unresectable solid tumor in a subject by administering an effective amount of a multi-specific binding protein, or a pharmaceutical formulation thereof. The multi-specific binding protein incorporates: a first antigen binding site that binds NKG2D including a VH with the CDR1, CDR2, and CDR3 sequences of SEQ ID NOs: 81, 82, and 97 or 112, respectively; and a VL with the CDR1, CDR2, and CDR3 sequences of SEQ ID NOs: 86, 77, and 87, respectively; a second antigen binding site that binds EGFR including (i) a VH having the CDR1, CDR2, and CDR3 sequences of SEQ ID NOs: 136, 157, and 138, respectively; and a VL having the CDR1, CDR2, and CDR3 sequences of SEQ ID NOs: 140, 141, and 151, respectively; (ii) a VH having the CDR1, CDR2, and CDR3 sequences of SEQ ID NOs: 136, 146, and 138, respectively; and a VL having the CDR1, CDR2, and CDR3 sequences of SEQ ID NOs: 140, 141, and 142, respectively; (iii) a VH having the CDR1, CDR2, and CDR3 sequences of SEQ ID NOs: 136, 146, and 138, respectively; and a VL having the CDR1, CDR2, and CDR3 sequences of SEQ ID NOs: 140, 141, and 151, respectively; (iv) a VH having the CDR1, CDR2, and CDR3 sequences of SEQ ID NOs: 136, 137, and 138, respectively; and a VL having the CDR1, CDR2, and CDR3 sequences of SEQ ID NOs: 140, 141, and 151, respectively; or (v) a VH having the CDR1, CDR2, and CDR3 sequences of SEQ ID NOs: 136, 137, and 138, respectively; and a VL having the CDR1, CDR2, and CDR3 sequences of SEQ ID NOs: 140, 141, and 142, respectively; and an antibody Fc domain or a portion thereof sufficient to bind CD16, or a third antigen-binding site that binds CD16.

In some embodiments, provided herein are methods of treating an unresectable solid tumor in a subject by administering an effective amount of a multi-specific binding protein, or a pharmaceutical formulation thereof. The multi-specific binding protein incorporates: a first polypeptide having the amino acid sequence of SEQ ID NO:167; (b) a second polypeptide having the amino acid sequence of SEQ ID NO:164; and (c) a third polypeptide having the amino acid sequence of SEQ ID NO:165.

In some embodiments, pharmaceutical formulations suitable for use in treating an unresectable solid tumor in a subject have 5 mg/mL to 50 mg/mL (e.g., 5 mg/mL to 50 mg/mL, 5 mg/mL to 45 mg/mL, 5 mg/mL to 40 mg/mL, 5 mg/mL to 35 mg/mL, 5 mg/mL to 30 mg/mL, 5 mg/mL to 25 mg/mL, 5 mg/mL to 20 mg/mL, 5 mg/mL to 15 mg/mL, 5 mg/mL to 10 mg/mL, 10 mg/mL to 50 mg/mL, 15 mg/mL to 50 mg/mL, 20 mg/mL to 50 mg/mL, 25 mg/mL to 50 mg/mL, 30 mg/mL to 50 mg/mL, 35 mg/mL to 50 mg/mL, 40 mg/mL to 50 mg/mL, 45 mg/mL to 50 mg/mL, or 15 mg/mL) of the multi-specific binding protein, and also include: 15 mM to 25 mM citrate (e.g., 15 mM to 25 mM, 16 mM to 25 mM, 17 mM to 25 mM, 18 mM to 25 mM, 19 mM to 25 mM, 20 mM to 25 mM, 21 mM to 25 mM, 22 mM to 25 mM, 23 mM to 25 mM, 24 mM to 25 mM, 15 mM to 24 mM, 15 mM to 23 mM, 15 mM to 22 mM, 15 mM to 23 mM, 15 mM to 22 mM, 15 mM to 21 mM, 15 mM to 20 mM, 15 mM to 19 mM, 15 mM to 18 mM, 15 mM to 17 mM, 15 mM to 16 mM, or about 20 mM), 4% to 8% (w/v) mannitol (e.g., 4% to 8% (w/v), 4% to 7% (w/v), 4% to 6% (w/v), 4% to 5% (w/v), 5% to 8% (w/v), 6% to 8% (w/v), 7% to 8% (w/v), or about 6% (w/v)); and 0.005% to 0.05% (w/v) polysorbate 80 (e.g., 0.005% to 0.05% (w/v), 0.005% to 0.04% (w/v), 0.005% to 0.03% (w/v), 0.005% to 0.02% (w/v), 0.005% to 0.01% (w/v), 0.006% to 0.05% (w/v), 0.007% to 0.05% (w/v), 0.008% to 0.05% (w/v), 0.009% to 0.05% (w/v), 0.01% to 0.05% (w/v), 0.02% to 0.05% (w/v), 0.03% to 0.05% (w/v), 0.04% to 0.05% (w/v), or 0.01% (w/v)), at pH 6.2 to 6.8 (e.g., 6.2 to 6.8, 6.2 to 6.7, 6.2 to 6.6, 6.2 to 6.5, 6.2 to 6.4, 6.2 to 6.3, 6.3 to 6.8, 6.4 to 6.8, 6.5 to 6.8, 6.6 to 6.8, 6.7 to 6.8, or about 6.5). In some embodiments, pharmaceutical formulations suitable for use in treating an unresectable solid tumor in a subject have about 15 mg/mL of the multi-specific binding protein, and also include: about 20 mM citrate, about 6% (w/v) mannitol; and about 0.01% (w/v) polysorbate 80, at about pH 6.5.

In some embodiments, provided herein are methods of treating a recurrent solid tumor in a subject by administering an effective amount of a multi-specific binding protein, or a pharmaceutical formulation thereof. The multi-specific binding protein incorporates: a first antigen-binding site that binds NKG2D; a second antigen-binding site that binds EGFR; and an antibody Fc domain or a portion thereof sufficient to bind CD16, or a third antigen-binding site that binds CD16.

In some embodiments, provided herein are methods of treating a recurrent solid tumor in a subject by administering an effective amount of a multi-specific binding protein, or a pharmaceutical formulation thereof. The multi-specific binding protein incorporates: a first antigen binding site that binds NKG2D; a second antigen binding site that binds EGFR including (i) a VH having the CDR1, CDR2, and CDR3 sequences of SEQ ID NOs: 136, 157, and 138, respectively; and a VL having the CDR1, CDR2, and CDR3 sequences of SEQ ID NOs: 140, 141, and 151, respectively; (ii) a VH having the CDR1, CDR2, and CDR3 sequences of SEQ ID NOs: 136, 146, and 138, respectively; and a VL having the CDR1, CDR2, and CDR3 sequences of SEQ ID NOs: 140, 141, and 142, respectively; (iii) a VH having the CDR1, CDR2, and CDR3 sequences of SEQ ID NOs: 136, 146, and 138, respectively; and a VL having the CDR1, CDR2, and CDR3 sequences of SEQ ID NOs: 140, 141, and 151, respectively; (iv) a VH having the CDR1, CDR2, and CDR3 sequences of SEQ ID NOs: 136, 137, and 138, respectively; and a VL having the CDR1, CDR2, and CDR3 sequences of SEQ ID NOs: 140, 141, and 151, respectively; or (v) a VH having the CDR1, CDR2, and CDR3 sequences of SEQ ID NOs: 136, 137, and 138, respectively; and a VL having the CDR1, CDR2, and CDR3 sequences of SEQ ID NOs: 140, 141, and 142, respectively; and an antibody Fc domain or a portion thereof sufficient to bind CD16, or a third antigen-binding site that binds CD16.

In some embodiments, provided herein are methods of treating a recurrent solid tumor in a subject by administering an effective amount of a multi-specific binding protein, or a pharmaceutical formulation thereof. The multi-specific binding protein incorporates: a first antigen binding site that binds NKG2D with a VH having the CDR1, CDR2, and CDR3 sequences of SEQ ID NOs: 81, 82, and 97 or 112, respectively; and a VL having the CDR1, CDR2, and CDR3 sequences of SEQ ID NOs: 86, 77, and 87, respectively; a second antigen binding site that binds EGFR with (i) a VH having the CDR1, CDR2, and CDR3 sequences of SEQ ID NOs: 136, 157, and 138, respectively; and a VL having the CDR1, CDR2, and CDR3 sequences of SEQ ID NOs: 140, 141, and 151, respectively; (ii) a VH having the CDR1, CDR2, and CDR3 sequences of SEQ ID NOs: 136, 146, and 138, respectively; and a VL having the CDR1, CDR2, and CDR3 sequences of SEQ ID NOs: 140, 141, and 142, respectively; (iii) a VH having the CDR1, CDR2, and CDR3 sequences of SEQ ID NOs: 136, 146, and 138, respectively; and a VL having the CDR1, CDR2, and CDR3 sequences of SEQ ID NOs: 140, 141, and 151, respectively; (iv) a VH having the CDR1, CDR2, and CDR3 sequences of SEQ ID NOs: 136, 137, and 138, respectively; and a VL having the CDR1, CDR2, and CDR3 sequences of SEQ ID NOs: 140, 141, and 151, respectively; or (v) a VH having the CDR1, CDR2, and CDR3 sequences of SEQ ID NOs: 136, 137, and 138, respectively; and a VL having the CDR1, CDR2, and CDR3 sequences of SEQ ID NOs: 140, 141, and 142, respectively; and an antibody Fc domain or a portion thereof sufficient to bind CD16, or a third antigen-binding site that binds CD16.

In some embodiments, provided herein are methods of treating a recurrent solid tumor in a subject by administering an effective amount of a multi-specific binding protein, or a pharmaceutical formulation thereof The multi-specific binding protein incorporates: a first polypeptide with the amino acid sequence of SEQ ID NO:167; (b) a second polypeptide with the amino acid sequence of SEQ ID NO:164; and (c) a third polypeptide with the amino acid sequence of SEQ ID NO:165.

In some embodiments, pharmaceutical formulations suitable for use in treating a recurrent solid tumor in a subject have 5 mg/mL to 50 mg/mL of the multi-specific binding protein (e.g., 5 mg/mL to 50 mg/mL, 5 mg/mL to 45 mg/mL, 5 mg/mL to 40 mg/mL, 5 mg/mL to 35 mg/mL, 5 mg/mL to 30 mg/mL, 5 mg/mL to 25 mg/mL, 5 mg/mL to 20 mg/mL, 5 mg/mL to 15 mg/mL, 5 mg/mL to 10 mg/mL, 10 mg/mL to 50 mg/mL, 15 mg/mL to 50 mg/mL, 20 mg/mL to 50 mg/mL, 25 mg/mL to 50 mg/mL, 30 mg/mL to 50 mg/mL, 35 mg/mL to 50 mg/mL, 40 mg/mL to 50 mg/mL, 45 mg/mL to 50 mg/mL, or 15 mg/mL), and also include: 15 mM to 25 mM citrate (e.g., 15 mM to 25 mM, 16 mM to 25 mM, 17 mM to 25 mM, 18 mM to 25 mM, 19 mM to 25 mM, 20 mM to 25 mM, 21 mM to 25 mM, 22 mM to 25 mM, 23 mM to 25 mM, 24 mM to 25 mM, 15 mM to 24 mM, 15 mM to 23 mM, 15 mM to 22 mM, 15 mM to 23 mM, 15 mM to 22 mM, 15 mM to 21 mM, 15 mM to 20 mM, 15 mM to 19 mM, 15 mM to 18 mM, 15 mM to 17 mM, 15 mM to 16 mM, or about 20 mM), 4% to 8% (w/v) mannitol (e.g., 4% to 8% (w/v), 4% to 7% (w/v), 4% to 6% (w/v), 4% to 5% (w/v), 5% to 8% (w/v), 6% to 8% (w/v), 7% to 8% (w/v), or about 6% (w/v)); and 0.005% to 0.05% (w/v) polysorbate 80 (e.g., 0.005% to 0.05% (w/v), 0.005% to 0.04% (w/v), 0.005% to 0.03% (w/v), 0.005% to 0.02% (w/v), 0.005% to 0.01% (w/v), 0.006% to 0.05% (w/v), 0.007% to 0.05% (w/v), 0.008% to 0.05% (w/v), 0.009% to 0.05% (w/v), 0.01% to 0.05% (w/v), 0.02% to 0.05% (w/v), 0.03% to 0.05% (w/v), 0.04% to 0.05% (w/v), or 0.01% (w/v)), at pH 6.2 to 6.8 (e.g., 6.2 to 6.8, 6.2 to 6.7, 6.2 to 6.6, 6.2 to 6.5, 6.2 to 6.4, 6.2 to 6.3, 6.3 to 6.8, 6.4 to 6.8, 6.5 to 6.8, 6.6 to 6.8, 6.7 to 6.8, or about 6.5). In some embodiments, pharmaceutical formulations suitable for use in treating a recurrent solid tumor in a subject include about 15 mg/mL of the multi-specific binding protein, and also include: about 20 mM citrate, about 6% (w/v) mannitol; and about 0.01% (w/v) polysorbate 80, at about pH 6.5.

In some embodiments, provided herein are methods of treating an advanced solid tumor, for which there is no effective standard therapy, in a subject by administering an effective amount of a multi-specific binding protein, or a pharmaceutical formulation thereof. The multi-specific binding protein incorporates: a first antigen-binding site that binds NKG2D; a second antigen-binding site that binds EGFR; and an antibody Fc domain or a portion thereof sufficient to bind CD16, or a third antigen-binding site that binds CD16.

In some embodiments, provided herein are methods of treating an advanced solid tumor, for which there is no effective standard therapy, in a subject by administering an effective amount of a multi-specific binding protein, or a pharmaceutical formulation thereof. The multi-specific binding protein incorporates: a first antigen binding site that binds NKG2D; a second antigen binding site that binds EGFR with (i) a VH having the CDR1, CDR2, and CDR3 sequences of SEQ ID NOs: 136, 157, and 138, respectively; and a VL having the CDR1, CDR2, and CDR3 sequences of SEQ ID NOs: 140, 141, and 151, respectively; (ii) a VH having the CDR1, CDR2, and CDR3 sequences of SEQ ID NOs: 136, 146, and 138, respectively; and a VL having the CDR1, CDR2, and CDR3 sequences of SEQ ID NOs: 140, 141, and 142, respectively; (iii) a VH having the CDR1, CDR2, and CDR3 sequences of SEQ ID NOs: 136, 146, and 138, respectively; and a VL having the CDR1, CDR2, and CDR3 sequences of SEQ ID NOs: 140, 141, and 151, respectively; (iv) a VH having the CDR1, CDR2, and CDR3 sequences of SEQ ID NOs: 136, 137, and 138, respectively; and a VL having the CDR1, CDR2, and CDR3 sequences of SEQ ID NOs: 140, 141, and 151, respectively; or (v) a VH having the CDR1, CDR2, and CDR3 sequences of SEQ ID NOs: 136, 137, and 138, respectively; and a VL having the CDR1, CDR2, and CDR3 sequences of SEQ ID NOs: 140, 141, and 142, respectively; and an antibody Fc domain or a portion thereof sufficient to bind CD16, or a third antigen-binding site that binds CD16.

In some embodiments, provided herein are methods of treating an advanced solid tumor, for which there is no effective standard therapy, in a subject by administering an effective amount of a multi-specific binding protein, or a pharmaceutical formulation thereof. The multi-specific binding protein incorporates: a first antigen binding site that binds NKG2D with a VH having the CDR1, CDR2, and CDR3 sequences of SEQ ID NOs: 81, 82, and 97 or 112, respectively; and a VL having the CDR1, CDR2, and CDR3 sequences of SEQ ID NOs: 86, 77, and 87, respectively; a second antigen binding site that binds EGFR with (i) a VH having the CDR1, CDR2, and CDR3 sequences of SEQ ID NOs: 136, 157, and 138, respectively; and a VL having the CDR1, CDR2, and CDR3 sequences of SEQ ID NOs: 140, 141, and 151, respectively; (ii) a VH having the CDR1, CDR2, and CDR3 sequences of SEQ ID NOs: 136, 146, and 138, respectively; and a VL having the CDR1, CDR2, and CDR3 sequences of SEQ ID NOs: 140, 141, and 142, respectively; (iii) a VH having the CDR1, CDR2, and CDR3 sequences of SEQ ID NOs: 136, 146, and 138, respectively; and a VL having the CDR1, CDR2, and CDR3 sequences of SEQ ID NOs: 140, 141, and 151, respectively; (iv) a VH having the CDR1, CDR2, and CDR3 sequences of SEQ ID NOs: 136, 137, and 138, respectively; and a VL having the CDR1, CDR2, and CDR3 sequences of SEQ ID NOs: 140, 141, and 151, respectively; or (v) a VH having the CDR1, CDR2, and CDR3 sequences of SEQ ID NOs: 136, 137, and 138, respectively; and a VL having the CDR1, CDR2, and CDR3 sequences of SEQ ID NOs: 140, 141, and 142, respectively; and an antibody Fc domain or a portion thereof sufficient to bind CD16, or a third antigen-binding site that binds CD16.

In some embodiments, provided herein are methods of treating an advanced solid tumor, for which there is no effective standard therapy, in a subject by administering an effective amount of a multi-specific binding protein, or a pharmaceutical formulation thereof. The multi-specific binding protein incorporates: a first polypeptide with the amino acid sequence of SEQ ID NO:167; (b) a second polypeptide with the amino acid sequence of SEQ ID NO:164; and (c) a third polypeptide with the amino acid sequence of SEQ ID NO:165.

In some embodiments, pharmaceutical formulations suitable for use in treating an advanced solid tumor, for which there is no effective standard therapy, in a subject have 5 mg/mL to 50 mg/mL of the multi-specific binding protein (e.g., 5 mg/mL to 50 mg/mL, 5 mg/mL to 45 mg/mL, 5 mg/mL to 40 mg/mL, 5 mg/mL to 35 mg/mL, 5 mg/mL to 30 mg/mL, 5 mg/mL to 25 mg/mL, 5 mg/mL to 20 mg/mL, 5 mg/mL to 15 mg/mL, 5 mg/mL to 10 mg/mL, 10 mg/mL to 50 mg/mL, 15 mg/mL to 50 mg/mL, 20 mg/mL to 50 mg/mL, 25 mg/mL to 50 mg/mL, 30 mg/mL to 50 mg/mL, 35 mg/mL to 50 mg/mL, 40 mg/mL to 50 mg/mL, 45 mg/mL to 50 mg/mL, or 15 mg/mL), and also include: 15 mM to 25 mM citrate (e.g., 15 mM to 25 mM, 16 mM to 25 mM, 17 mM to 25 mM, 18 mM to 25 mM, 19 mM to 25 mM, 20 mM to 25 mM, 21 mM to 25 mM, 22 mM to 25 mM, 23 mM to 25 mM, 24 mM to 25 mM, 15 mM to 24 mM, 15 mM to 23 mM, 15 mM to 22 mM, 15 mM to 23 mM, 15 mM to 22 mM, 15 mM to 21 mM, 15 mM to 20 mM, 15 mM to 19 mM, 15 mM to 18 mM, 15 mM to 17 mM, 15 mM to 16 mM, or about 20 mM), 4% to 8% (w/v) mannitol (e.g., 4% to 8% (w/v), 4% to 7% (w/v), 4% to 6% (w/v), 4% to 5% (w/v), 5% to 8% (w/v), 6% to 8% (w/v), 7% to 8% (w/v), or about 6% (w/v)); and 0.005% to 0.05% (w/v) polysorbate 80 (e.g., 0.005% to 0.05% (w/v), 0.005% to 0.04% (w/v), 0.005% to 0.03% (w/v), 0.005% to 0.02% (w/v), 0.005% to 0.01% (w/v), 0.006% to 0.05% (w/v), 0.007% to 0.05% (w/v), 0.008% to 0.05% (w/v), 0.009% to 0.05% (w/v), 0.01% to 0.05% (w/v), 0.02% to 0.05% (w/v), 0.03% to 0.05% (w/v), 0.04% to 0.05% (w/v), or 0.01% (w/v)), at pH 6.2 to 6.8 (e.g., 6.2 to 6.8, 6.2 to 6.7, 6.2 to 6.6, 6.2 to 6.5, 6.2 to 6.4, 6.2 to 6.3, 6.3 to 6.8, 6.4 to 6.8, 6.5 to 6.8, 6.6 to 6.8, 6.7 to 6.8, or about 6.5). In some embodiments, pharmaceutical formulations suitable for use in treating an advanced solid tumor, for which there is no effective standard therapy, in a subject include about 15 mg/mL of the multi-specific binding protein, and further also include: about 20 mM citrate, about 6% (w/v) mannitol; and about 0.01% (w/v) polysorbate 80, at about pH 6.5.

In some embodiments, provided herein are methods of treating cancer in a subject that is intolerant of standard therapies by administering an effective amount of a multi-specific binding protein, or a pharmaceutical formulation thereof. The multi-specific binding protein incorporates: a first antigen-binding site that binds NKG2D; a second antigen-binding site that binds EGFR; and an antibody Fc domain or a portion thereof sufficient to bind CD16, or a third antigen-binding site that binds CD16.

In some embodiments, provided herein are methods of treating cancer in a subject that is intolerant of standard therapies by administering an effective amount of a multi-specific binding protein, or a pharmaceutical formulation thereof. The multi-specific binding protein incorporates: a first antigen binding site that binds NKG2D; a second antigen binding site that binds EGFR with (i) a VH having the CDR1, CDR2, and CDR3 sequences of SEQ ID NOs: 136, 157, and 138, respectively; and a VL having the CDR1, CDR2, and CDR3 sequences of SEQ ID NOs: 140, 141, and 151, respectively; (ii) a VH having the CDR1, CDR2, and CDR3 sequences of SEQ ID NOs: 136, 146, and 138, respectively; and a VL having the CDR1, CDR2, and CDR3 sequences of SEQ ID NOs: 140, 141, and 142, respectively; (iii) a VH having the CDR1, CDR2, and CDR3 sequences of SEQ ID NOs: 136, 146, and 138, respectively; and a VL having the CDR1, CDR2, and CDR3 sequences of SEQ ID NOs: 140, 141, and 151, respectively; (iv) a VH having the CDR1, CDR2, and CDR3 sequences of SEQ ID NOs: 136, 137, and 138, respectively; and a VL having the CDR1, CDR2, and CDR3 sequences of SEQ ID NOs: 140, 141, and 151, respectively; or (v) a VH having the CDR1, CDR2, and CDR3 sequences of SEQ ID NOs: 136, 137, and 138, respectively; and a VL having the CDR1, CDR2, and CDR3 sequences of SEQ ID NOs: 140, 141, and 142, respectively; and an antibody Fc domain or a portion thereof sufficient to bind CD16, or a third antigen-binding site that binds CD16.

In some embodiments, provided herein are methods of treating cancer in a subject that is intolerant of standard therapies by administering an effective amount of a multi-specific binding protein, or a pharmaceutical formulation thereof. The multi-specific binding protein incorporates: a first antigen binding site that binds NKG2D with a VH including the CDR1, CDR2, and CDR3 sequences of SEQ ID NOs: 81, 82, and 97 or 112, respectively; and a VL including the CDR1, CDR2, and CDR3 sequences of SEQ ID NOs: 86, 77, and 87, respectively; a second antigen binding site that binds EGFR with (i) a VH having the CDR1, CDR2, and CDR3 sequences of SEQ ID NOs: 136, 157, and 138, respectively; and a VL having the CDR1, CDR2, and CDR3 sequences of SEQ ID NOs: 140, 141, and 151, respectively; (ii) a VH having the CDR1, CDR2, and CDR3 sequences of SEQ ID NOs: 136, 146, and 138, respectively; and a VL having the CDR1, CDR2, and CDR3 sequences of SEQ ID NOs: 140, 141, and 142, respectively; (iii) a VH having the CDR1, CDR2, and CDR3 sequences of SEQ ID NOs: 136, 146, and 138, respectively; and a VL having the CDR1, CDR2, and CDR3 sequences of SEQ ID NOs: 140, 141, and 151, respectively; (iv) a VH having the CDR1, CDR2, and CDR3 sequences of SEQ ID NOs: 136, 137, and 138, respectively; and a VL having the CDR1, CDR2, and CDR3 sequences of SEQ ID NOs: 140, 141, and 151, respectively; or (v) a VH having the CDR1, CDR2, and CDR3 sequences of SEQ ID NOs: 136, 137, and 138, respectively; and a VL having the CDR1, CDR2, and CDR3 sequences of SEQ ID NOs: 140, 141, and 142, respectively; and an antibody Fc domain or a portion thereof sufficient to bind CD16, or a third antigen-binding site that binds CD16.

In some embodiments, provided herein are methods of treating cancer in a subject that is intolerant of standard therapies by administering an effective amount of a multi-specific binding protein, or a pharmaceutical formulation thereof. The multi-specific binding protein incorporates: a first polypeptide with the amino acid sequence of SEQ ID NO:167; (b) a second polypeptide with the amino acid sequence of SEQ ID NO:164; and (c) a third polypeptide with the amino acid sequence of SEQ ID NO:165.

In some embodiments, pharmaceutical formulations suitable for use in treating cancer in a subject that is intolerant of standard therapies have 5 mg/mL to 50 mg/mL of the multi-specific binding protein (e.g., 5 mg/mL to 50 mg/mL, 5 mg/mL to 45 mg/mL, 5 mg/mL to 40 mg/mL, 5 mg/mL to 35 mg/mL, 5 mg/mL to 30 mg/mL, 5 mg/mL to 25 mg/mL, 5 mg/mL to 20 mg/mL, 5 mg/mL to 15 mg/mL, 5 mg/mL to 10 mg/mL, 10 mg/mL to 50 mg/mL, 15 mg/mL to 50 mg/mL, 20 mg/mL to 50 mg/mL, 25 mg/mL to 50 mg/mL, 30 mg/mL to 50 mg/mL, 35 mg/mL to 50 mg/mL, 40 mg/mL to 50 mg/mL, 45 mg/mL to 50 mg/mL, or 15 mg/mL), and also include: 15 mM to 25 mM citrate (e.g., 15 mM to 25 mM, 16 mM to 25 mM, 17 mM to 25 mM, 18 mM to 25 mM, 19 mM to 25 mM, 20 mM to 25 mM, 21 mM to 25 mM, 22 mM to 25 mM, 23 mM to 25 mM, 24 mM to 25 mM, 15 mM to 24 mM, 15 mM to 23 mM, 15 mM to 22 mM, 15 mM to 23 mM, 15 mM to 22 mM, 15 mM to 21 mM, 15 mM to 20 mM, 15 mM to 19 mM, 15 mM to 18 mM, 15 mM to 17 mM, 15 mM to 16 mM, or about 20 mM), 4% to 8% (w/v) mannitol (e.g., 4% to 8% (w/v), 4% to 7% (w/v), 4% to 6% (w/v), 4% to 5% (w/v), 5% to 8% (w/v), 6% to 8% (w/v), 7% to 8% (w/v), or about 6% (w/v)); and 0.005% to 0.05% (w/v) polysorbate 80 (e.g., 0.005% to 0.05% (w/v), 0.005% to 0.04% (w/v), 0.005% to 0.03% (w/v), 0.005% to 0.02% (w/v), 0.005% to 0.01% (w/v), 0.006% to 0.05% (w/v), 0.007% to 0.05% (w/v), 0.008% to 0.05% (w/v), 0.009% to 0.05% (w/v), 0.01% to 0.05% (w/v), 0.02% to 0.05% (w/v), 0.03% to 0.05% (w/v), 0.04% to 0.05% (w/v), or 0.01% (w/v)), at pH 6.2 to 6.8 (e.g., 6.2 to 6.8, 6.2 to 6.7, 6.2 to 6.6, 6.2 to 6.5, 6.2 to 6.4, 6.2 to 6.3, 6.3 to 6.8, 6.4 to 6.8, 6.5 to 6.8, 6.6 to 6.8, 6.7 to 6.8, or about 6.5). In some embodiments, pharmaceutical formulations suitable for use in treating cancer in a subject that is intolerant of standard therapies include about 15 mg/mL of the multi-specific binding protein, and also include: about 20 mM citrate, about 6% (w/v) mannitol; and about 0.01% (w/v) polysorbate 80, at about pH 6.5.

In some embodiments, provided herein are methods for treating cancer in a subject, where the subject has:
histologically or cytologically proven locally advanced or metastatic solid tumors of epithelial origin for which the expression of EGFR has been reported in the literature or who carry an activating EGFR mutation, for which no standard therapy exists, or standard therapy has failed;
adequate hematological function defined by white blood cell (WBC) count>3×109/L, with absolute neutrophil count (ANC)>1.5×109/L, lymphocyte count>0.5×109/L, platelet count>75×109/L, and hemoglobin>9 g/dL (may have been transfused);
adequate hepatic function defined by a total bilirubin level<1.5× the upper limit of normal (ULN), an aspartate aminotransferase (AST) level<2.5×ULN, and an alanine aminotransferase (ALT) level<2.5×ULN, or, for patients with documented metastatic disease to the liver, AST and ALT levels<5×ULN; and
adequate renal function defined by an estimated creatinine clearance>50 mL/min according to the Cockcroft-Gault formula.

In some embodiments, provided herein are methods of treating cancer in a subject by administering nivolumab in combination with an effective amount of a multi-specific binding protein, or a pharmaceutical formulation thereof. The multi-specific binding protein incorporates: a first antigen-binding site that binds NKG2D; a second antigen-binding site that binds EGFR; and an antibody Fe domain or a portion thereof sufficient to bind CD16, or a third antigen-binding site that binds CD16.

In some embodiments, provided herein are methods of treating cancer in a subject by administering nivolumab in combination with an effective amount of a multi-specific binding protein, or a pharmaceutical formulation thereof. The multi-specific binding protein incorporates: a first antigen binding site that binds NKG2D; a second antigen binding site that binds EGFR with (i) a VH having the CDR1, CDR2, and CDR3 sequences of SEQ ID NOs: 136, 157, and 138, respectively; and a VL having the CDR1, CDR2, and CDR3 sequences of SEQ ID NOs: 140, 141, and 151, respectively; (ii) a VH having the CDR1, CDR2, and CDR3 sequences of SEQ ID NOs: 136, 146, and 138, respectively; and a VL having the CDR1, CDR2, and CDR3 sequences of SEQ ID NOs: 140, 141, and 142, respectively; (iii) a VH having the CDR1, CDR2, and CDR3 sequences of SEQ ID NOs: 136, 146, and 138, respectively; and a VL having the CDR1, CDR2, and CDR3 sequences of SEQ ID NOs: 140, 141, and 151, respectively; (iv) a VH having the CDR1, CDR2, and CDR3 sequences of SEQ ID NOs: 136, 137, and 138, respectively; and a VL having the CDR1, CDR2, and CDR3 sequences of SEQ ID NOs: 140, 141, and 151, respectively; or (v) a VH having the CDR1, CDR2, and CDR3 sequences of SEQ ID NOs: 136, 137, and 138, respectively; and a VL having the CDR1, CDR2, and CDR3 sequences of SEQ ID NOs: 140, 141, and 142, respectively; and an antibody Fc domain or a portion thereof sufficient to bind CD16, or a third antigen-binding site that binds CD16.

In some embodiments, provided herein are methods of treating cancer in a subject by administering nivolumab in combination with an effective amount of a multi-specific binding protein, or a pharmaceutical formulation thereof. The multi-specific binding protein incorporates: a first antigen binding site that binds NKG2D with a VH including the CDR1, CDR2, and CDR3 sequences of SEQ ID NOs: 81, 82, and 97 or 112, respectively; and a VL including the CDR1, CDR2, and CDR3 sequences of SEQ ID NOs: 86, 77, and 87, respectively; a second antigen binding site that binds EGFR with (i) a VH having the CDR1, CDR2, and CDR3 sequences of SEQ ID NOs: 136, 157, and 138, respectively; and a VL having the CDR1, CDR2, and CDR3 sequences of SEQ ID NOs: 140, 141, and 151, respectively; (ii) a VH having the CDR1, CDR2, and CDR3 sequences of SEQ ID NOs: 136, 146, and 138, respectively; and a VL having the CDR1, CDR2, and CDR3 sequences of SEQ ID NOs: 140, 141, and 142, respectively; (iii) a VH having the CDR1, CDR2, and CDR3 sequences of SEQ ID NOs: 136, 146, and 138, respectively; and a VL having the CDR1, CDR2, and CDR3 sequences of SEQ ID NOs: 140, 141, and 151, respectively; (iv) a VH having the CDR1, CDR2, and CDR3 sequences of SEQ ID NOs: 136, 137, and 138, respectively; and a VL having the CDR1, CDR2, and CDR3 sequences of SEQ ID NOs: 140, 141, and 151, respectively; or (v) a VH having the CDR1, CDR2, and CDR3 sequences of SEQ ID NOs: 136, 137, and 138, respectively; and a VL having the CDR1, CDR2, and CDR3 sequences of SEQ ID NOs: 140, 141, and 142, respectively; and an antibody Fc domain or a portion thereof sufficient to bind CD16, or a third antigen-binding site that binds CD16.

In some embodiments, provided herein are methods of treating cancer in a subject by administering nivolumab in combination with an effective amount of a multi-specific binding protein, or a pharmaceutical formulation thereof. The multi-specific binding protein incorporates: a first polypeptide with the amino acid sequence of SEQ ID NO:167; (b) a second polypeptide with the amino acid sequence of SEQ ID NO:164; and (c) a third polypeptide with the amino acid sequence of SEQ ID NO:165.

In some embodiments, pharmaceutical formulations suitable for use in treating cancer in a subject, in combination with nivolumab, have 5 mg/mL to 50 mg/mL of the multi-specific binding protein (e.g., 5 mg/mL to 50 mg/mL, 5 mg/mL to 45 mg/mL, 5 mg/mL to 40 mg/mL, 5 mg/mL to 35 mg/mL, 5 mg/mL to 30 mg/mL, 5 mg/mL to 25 mg/mL, 5 mg/mL to 20 mg/mL, 5 mg/mL to 15 mg/mL, 5 mg/mL to 10 mg/mL, 10 mg/mL to 50 mg/mL, 15 mg/mL to 50 mg/mL, 20 mg/mL to 50 mg/mL, 25 mg/mL to 50 mg/mL, 30 mg/mL to 50 mg/mL, 35 mg/mL to 50 mg/mL, 40 mg/mL to 50 mg/mL, 45 mg/mL to 50 mg/mL, or 15 mg/mL), and also include: 15 mM to 25 mM citrate (e.g., 15 mM to 25 mM, 16 mM to 25 mM, 17 mM to 25 mM, 18 mM to 25 mM, 19 mM to 25 mM, 20 mM to 25 mM, 21 mM to 25 mM, 22 mM to 25 mM, 23 mM to 25 mM, 24 mM to 25 mM, 15 mM to 24 mM, 15 mM to 23 mM, 15 mM to 22 mM, 15 mM to 23 mM, 15 mM to 22 mM, 15 mM to 21 mM, 15 mM to 20 mM, 15 mM to 19 mM, 15 mM to 18 mM, 15 mM to 17 mM, 15 mM to 16 mM, or about 20 mM), 4% to 8% (w/v) mannitol (e.g., 4% to 8% (w/v), 4% to 7% (w/v), 4% to 6% (w/v), 4% to 5% (w/v), 5% to 8% (w/v), 6% to 8% (w/v), 7% to 8% (w/v), or about 6% (w/v)); and 0.005% to 0.05% (w/v) polysorbate 80 (e.g., 0.005% to 0.05% (w/v), 0.005% to 0.04% (w/v), 0.005% to 0.03% (w/v), 0.005% to 0.02% (w/v), 0.005% to 0.01% (w/v), 0.006% to 0.05% (w/v), 0.007% to 0.05% (w/v), 0.008% to 0.05% (w/v), 0.009% to 0.05% (w/v), 0.01% to 0.05% (w/v), 0.02% to 0.05% (w/v), 0.03% to 0.05% (w/v), 0.04% to 0.05% (w/v), or 0.01% (w/v)), at pH 6.2 to 6.8 (e.g., 6.2 to 6.8, 6.2 to 6.7, 6.2 to 6.6, 6.2 to 6.5, 6.2 to 6.4, 6.2 to 6.3, 6.3 to 6.8, 6.4 to 6.8, 6.5 to 6.8, 6.6 to 6.8, 6.7 to 6.8, or about 6.5). In some embodiments, pharmaceutical formulations suitable for use in treating cancer in a subject, in combination with nivolumab, include about 15 mg/mL of the multi-specific binding protein, and also include: about 20 mM citrate, about 6% (w/v) mannitol; and about 0.01% (w/v) polysorbate 80, at about pH 6.5. In some embodiments, provided herein are methods for treating cancer in a subject, where the subject:
is eligible to receive nivolumab per its label for a malignancy of epithelial origin; or
is a patent for which no standard therapy exists or standard therapy has failed for a malignancy of epithelial origin.

In some embodiments, provided herein are methods of treating cancer in a subject by administering 5 mg/kg to 50 mg/kg (e.g., 5 mg/kg to 50 mg/kg, 5 mg/kg to 45 mg/kg, 5 mg/kg to 40 mg/kg, 5 mg/kg to 35 mg/kg, 5 mg/kg to 30 mg/kg, 5 mg/kg to 25 mg/kg, 5 mg/kg to 20 mg/kg, 5 mg/kg to 15 mg/kg, 5 mg/kg to 10 mg/kg, 10 mg/kg to 50 mg/kg, 10 mg/kg to 45 mg/kg, 10 mg/kg to 40 mg/kg, 10 mg/kg to 35 mg/kg, 10 mg/kg to 30 mg/kg, 10 mg/kg to 25 mg/kg, 10 mg/kg to 20 mg/kg, 10 mg/kg to 15 mg/kg, 15 mg/kg to 50 mg/kg, 15 mg/kg to 45 mg/kg, 15 mg/kg to 40 mg/kg, 15 mg/kg to 35 mg/kg, 15 mg/kg to 30 mg/kg, 15 mg/kg to 25 mg/kg, 15 mg/kg to 20 mg/kg, 20 mg/kg to 50 mg/kg, 20 mg/kg to 45 mg/kg, 20 mg/kg to 40 mg/kg, 20 mg/kg to 35 mg/kg, 20 mg/kg to 30 mg/kg, 20 mg/kg to 25 mg/kg, 25 mg/kg to 50 mg/kg, 25 mg/kg to 45 mg/kg, 25 mg/kg to 40 mg/kg, 25 mg/kg to 35 mg/kg, 25 mg/kg to 30 mg/kg, 30 mg/kg to 50 mg/kg, 30 mg/kg to 45 mg/kg, 30 mg/kg to 40 mg/kg, 30 mg/kg to 35 mg/kg, 35 mg/kg to 50 mg/kg, 35 mg/kg to 45 mg/kg, 35 mg/kg to 40 mg/kg, 40 mg/kg to 50 mg/kg, 40 mg/kg to 45 mg/kg, or 45 mg/kg to 50 mg/kg) of a multi-specific binding protein, or a pharmaceutical formulation thereof. In some embodiments, provided herein are methods of treating cancer in a subject by administering 5 mg/kg to 20 mg/kg of a multi-specific binding protein, or a pharmaceutical formulation thereof. The multi-specific binding protein incorporates: a first antigen-binding site that binds NKG2D; a second antigen-binding site that binds EGFR; and an antibody Fc domain or a portion thereof sufficient to bind CD16, or a third antigen-binding site that binds CD16.

In some embodiments, provided herein are methods of treating cancer in a subject by administering 5 mg/kg to 50 mg/kg (e.g., 5 mg/kg to 50 mg/kg, 5 mg/kg to 45 mg/kg, 5 mg/kg to 40 mg/kg, 5 mg/kg to 35 mg/kg, 5 mg/kg to 30 mg/kg, 5 mg/kg to 25 mg/kg, 5 mg/kg to 20 mg/kg, 5 mg/kg to 15 mg/kg, 5 mg/kg to 10 mg/kg, 10 mg/kg to 50 mg/kg, 10 mg/kg to 45 mg/kg, 10 mg/kg to 40 mg/kg, 10 mg/kg to 35 mg/kg, 10 mg/kg to 30 mg/kg, 10 mg/kg to 25 mg/kg, 10 mg/kg to 20 mg/kg, 10 mg/kg to 15 mg/kg, 15 mg/kg to 50 mg/kg, 15 mg/kg to 45 mg/kg, 15 mg/kg to 40 mg/kg, 15 mg/kg to 35 mg/kg, 15 mg/kg to 30 mg/kg, 15 mg/kg to 25 mg/kg, 15 mg/kg to 20 mg/kg, 20 mg/kg to 50 mg/kg, 20 mg/kg to 45 mg/kg, 20 mg/kg to 40 mg/kg, 20 mg/kg to 35 mg/kg, 20 mg/kg to 30 mg/kg, 20 mg/kg to 25 mg/kg, 25 mg/kg to 50 mg/kg, 25 mg/kg to 45 mg/kg, 25 mg/kg to 40 mg/kg, 25 mg/kg to 35 mg/kg, 25 mg/kg to 30 mg/kg, 30 mg/kg to 50 mg/kg, 30 mg/kg to 45 mg/kg, 30 mg/kg to 40 mg/kg, 30 mg/kg to 35 mg/kg, 35 mg/kg to 50 mg/kg, 35 mg/kg to 45 mg/kg, 35 mg/kg to 40 mg/kg, 40 mg/kg to 50 mg/kg, 40 mg/kg to 45 mg/kg, or 45 mg/kg to 50 mg/kg) of a multi-specific binding protein, or a pharmaceutical formulation thereof. In some embodiments, provided herein are methods of treating cancer in a subject by administering 5 mg/kg to 20 mg/kg of a multi-specific binding protein, or a pharmaceutical formulation thereof. The multi-specific binding protein incorporates: a first antigen binding site that binds NKG2D; a second antigen binding site that binds EGFR with (i) a VH having the CDR1, CDR2, and CDR3 sequences of SEQ ID NOs: 136, 157, and 138, respectively; and a VL having the CDR1, CDR2, and CDR3 sequences of SEQ ID NOs: 140, 141, and 151, respectively; (ii) a VH having the CDR1, CDR2, and CDR3 sequences of SEQ ID NOs: 136, 146, and 138, respectively; and a VL having the CDR1, CDR2, and CDR3 sequences of SEQ ID NOs: 140, 141, and 142, respectively; (iii) a VH having the CDR1, CDR2, and CDR3 sequences of SEQ ID NOs: 136, 146, and 138, respectively; and a VL having the CDR1, CDR2, and CDR3 sequences of SEQ ID NOs: 140, 141, and 151, respectively; (iv) a VH having the CDR1, CDR2, and CDR3 sequences of SEQ ID NOs: 136, 137, and 138, respectively; and a VL having the CDR1, CDR2, and CDR3 sequences of SEQ ID NOs: 140, 141, and 151, respectively; or (v) a VH having the CDR1, CDR2, and CDR3 sequences of SEQ ID NOs: 136, 137, and 138, respectively; and a VL having the CDR1, CDR2, and CDR3 sequences of SEQ ID NOs: 140, 141, and 142, respectively; and an antibody Fc domain or a portion thereof sufficient to bind CD16, or a third antigen-binding site that binds CD16.

In some embodiments, provided herein are methods of treating cancer in a subject by administering 5 mg/kg to 50 mg/kg (e.g., 5 mg/kg to 50 mg/kg, 5 mg/kg to 45 mg/kg, 5 mg/kg to 40 mg/kg, 5 mg/kg to 35 mg/kg, 5 mg/kg to 30 mg/kg, 5 mg/kg to 25 mg/kg, 5 mg/kg to 20 mg/kg, 5 mg/kg to 15 mg/kg, 5 mg/kg to 10 mg/kg, 10 mg/kg to 50 mg/kg, 10 mg/kg to 45 mg/kg, 10 mg/kg to 40 mg/kg, 10 mg/kg to 35 mg/kg, 10 mg/kg to 30 mg/kg, 10 mg/kg to 25 mg/kg, 10 mg/kg to 20 mg/kg, 10 mg/kg to 15 mg/kg, 15 mg/kg to 50 mg/kg, 15 mg/kg to 45 mg/kg, 15 mg/kg to 40 mg/kg, 15 mg/kg to 35 mg/kg, 15 mg/kg to 30 mg/kg, 15 mg/kg to 25 mg/kg, 15 mg/kg to 20 mg/kg, 20 mg/kg to 50 mg/kg, 20 mg/kg to 45 mg/kg, 20 mg/kg to 40 mg/kg, 20 mg/kg to 35 mg/kg, 20 mg/kg to 30 mg/kg, 20 mg/kg to 25 mg/kg, 25 mg/kg to 50 mg/kg, 25 mg/kg to 45 mg/kg, 25 mg/kg to 40 mg/kg, 25 mg/kg to 35 mg/kg, 25 mg/kg to 30 mg/kg, 30 mg/kg to 50 mg/kg, 30 mg/kg to 45 mg/kg, 30 mg/kg to 40 mg/kg, 30 mg/kg to 35 mg/kg, 35 mg/kg to 50 mg/kg, 35 mg/kg to 45 mg/kg, 35 mg/kg to 40 mg/kg, 40 mg/kg to 50 mg/kg, 40 mg/kg to 45 mg/kg, or 45 mg/kg to 50 mg/kg) of a multi-specific binding protein, or a pharmaceutical formulation thereof. In some embodiments, provided herein are methods of treating cancer in a subject by administering 5 mg/kg to 20 mg/kg of a multi-specific binding protein, or a pharmaceutical formulation thereof. The multi-specific binding protein incorporates: a first antigen binding site that binds NKG2D with a VH including the CDR1, CDR2, and CDR3 sequences of SEQ ID NOs: 81, 82, and 97 or 112, respectively; and a VL including the CDR1, CDR2, and CDR3 sequences of SEQ ID NOs: 86, 77, and 87, respectively; a second antigen binding site that binds EGFR with (i) a VH having the CDR1, CDR2, and CDR3 sequences of SEQ ID NOs: 136, 157, and 138, respectively; and a VL having the CDR1, CDR2, and CDR3 sequences of SEQ ID NOs: 140, 141, and 151, respectively; (ii) a VH having the CDR1, CDR2, and CDR3 sequences of SEQ ID NOs: 136, 146, and 138, respectively; and a VL having the CDR1, CDR2, and CDR3 sequences of SEQ ID NOs: 140, 141, and 142, respectively; (iii) a VH having the CDR1, CDR2, and CDR3 sequences of SEQ ID NOs: 136, 146, and 138, respectively; and a VL having the CDR1, CDR2, and CDR3 sequences of SEQ ID NOs: 140, 141, and 151, respectively; (iv) a VH having the CDR1, CDR2, and CDR3 sequences of SEQ ID NOs: 136, 137, and 138, respectively; and a VL having the CDR1, CDR2, and CDR3 sequences of SEQ ID NOs: 140, 141, and 151, respectively; or (v) a VH having the CDR1, CDR2, and CDR3 sequences of SEQ ID NOs: 136, 137, and 138, respectively; and a VL having the CDR1, CDR2, and CDR3 sequences of SEQ ID NOs: 140, 141, and 142, respectively; and an antibody Fc domain or a portion thereof sufficient to bind CD16, or a third antigen-binding site that binds CD16.

In some embodiments, provided herein are methods of treating cancer in a subject by administering 5 mg/kg to 50 mg/kg (e.g., 5 mg/kg to 50 mg/kg, 5 mg/kg to 45 mg/kg, 5 mg/kg to 40 mg/kg, 5 mg/kg to 35 mg/kg, 5 mg/kg to 30 mg/kg, 5 mg/kg to 25 mg/kg, 5 mg/kg to 20 mg/kg, 5 mg/kg to 15 mg/kg, 5 mg/kg to 10 mg/kg, 10 mg/kg to 50 mg/kg, 10 mg/kg to 45 mg/kg, 10 mg/kg to 40 mg/kg, 10 mg/kg to 35 mg/kg, 10 mg/kg to 30 mg/kg, 10 mg/kg to 25 mg/kg, 10 mg/kg to 20 mg/kg, 10 mg/kg to 15 mg/kg, 15 mg/kg to 50 mg/kg, 15 mg/kg to 45 mg/kg, 15 mg/kg to 40 mg/kg, 15 mg/kg to 35 mg/kg, 15 mg/kg to 30 mg/kg, 15 mg/kg to 25 mg/kg, 15 mg/kg to 20 mg/kg, 20 mg/kg to 50 mg/kg, 20 mg/kg to 45 mg/kg, 20 mg/kg to 40 mg/kg, 20 mg/kg to 35 mg/kg, 20 mg/kg to 30 mg/kg, 20 mg/kg to 25 mg/kg, 25 mg/kg to 50 mg/kg, 25 mg/kg to 45 mg/kg, 25 mg/kg to 40 mg/kg, 25 mg/kg to 35 mg/kg, 25 mg/kg to 30 mg/kg, 30 mg/kg to 50 mg/kg, 30 mg/kg to 45 mg/kg, 30 mg/kg to 40 mg/kg, 30 mg/kg to 35 mg/kg, 35 mg/kg to 50 mg/kg, 35 mg/kg to 45 mg/kg, 35 mg/kg to 40 mg/kg, 40 mg/kg to 50 mg/kg, 40 mg/kg to 45 mg/kg, or 45 mg/kg to 50 mg/kg) of a multi-specific binding protein, or a pharmaceutical formulation thereof. In some embodiments, provided herein are methods of treating cancer in a subject by administering 5 mg/kg to 20 mg/kg of a multi-specific binding protein, or a pharmaceutical formulation thereof. The multi-specific binding protein incorporates: a first polypeptide with the amino acid sequence of SEQ ID NO:167; (b) a second polypeptide with the amino acid sequence of SEQ ID NO:164; and (c) a third polypeptide with the amino acid sequence of SEQ ID NO:165.

In some embodiments, pharmaceutical formulations suitable for use in treating cancer in a subject by administration at 5 mg/kg to 50 mg/kg have 5 mg/mL to 50 mg/mL of the multi-specific binding protein (e.g., 5 mg/mL to 50 mg/mL, 5 mg/mL to 45 mg/mL, 5 mg/mL to 40 mg/mL, 5 mg/mL to 35 mg/mL, 5 mg/mL to 30 mg/mL, 5 mg/mL to 25 mg/mL, 5 mg/mL to 20 mg/mL, 5 mg/mL to 15 mg/mL, 5 mg/mL to 10 mg/mL, 10 mg/mL to 50 mg/mL, 15 mg/mL to 50 mg/mL, 20 mg/mL to 50 mg/mL, 25 mg/mL to 50 mg/mL, 30 mg/mL to 50 mg/mL, 35 mg/mL to 50 mg/mL, 40 mg/mL to 50 mg/mL, 45 mg/mL to 50 mg/mL, or 15 mg/mL), and also include: 15 mM to 25 mM citrate (e.g., 15 mM to 25 mM, 16 mM to 25 mM, 17 mM to 25 mM, 18 mM to 25 mM, 19 mM to 25 mM, 20 mM to 25 mM, 21 mM to 25 mM, 22 mM to 25 mM, 23 mM to 25 mM, 24 mM to 25 mM, 15 mM to 24 mM, 15 mM to 23 mM, 15 mM to 22 mM, 15 mM to 23 mM, 15 mM to 22 mM, 15 mM to 21 mM, 15 mM to 20 mM, 15 mM to 19 mM, 15 mM to 18 mM, 15 mM to 17 mM, 15 mM to 16 mM, or about 20 mM), 4% to 8% (w/v) mannitol (e.g., 4% to 8% (w/v), 4% to 7% (w/v), 4% to 6% (w/v), 4% to 5% (w/v), 5% to 8% (w/v), 6% to 8% (w/v), 7% to 8% (w/v), or about 6% (w/v)); and 0.005% to 0.05% (w/v) polysorbate 80 (e.g., 0.005% to 0.05% (w/v), 0.005% to 0.04% (w/v), 0.005% to 0.03% (w/v), 0.005% to 0.02% (w/v), 0.005% to 0.01% (w/v), 0.006% to 0.05% (w/v), 0.007% to 0.05% (w/v), 0.008% to 0.05% (w/v), 0.009% to 0.05% (w/v), 0.01% to 0.05% (w/v), 0.02% to 0.05% (w/v), 0.03% to 0.05% (w/v), 0.04% to 0.05% (w/v), or 0.01% (w/v)), at pH 6.2 to 6.8 (e.g., 6.2 to 6.8, 6.2 to 6.7, 6.2 to 6.6, 6.2 to 6.5, 6.2 to 6.4, 6.2 to 6.3, 6.3 to 6.8, 6.4 to 6.8, 6.5 to 6.8, 6.6 to 6.8, 6.7 to 6.8, or about 6.5). In some embodiments, pharmaceutical formulations suitable for use in treating cancer in a subject by administration at 5 mg/kg to 50 mg/kg include about 15 mg/mL of the multi-specific binding protein, and also include: about 20 mM citrate, about 6% (w/v) mannitol; and about 0.01% (w/v) polysorbate 80, at about pH 6.5.

In some embodiments, provided herein are methods of treating cancer in a subject by administering an effective amount of a multi-specific binding protein, or a pharmaceutical formulation thereof, once weekly in 4-week treatment cycles. The multi-specific binding protein incorporates: a first antigen-binding site that binds NKG2D; a second antigen-binding site that binds EGFR; and an antibody Fc domain or a portion thereof sufficient to bind CD16, or a third antigen-binding site that binds CD16.

In some embodiments, provided herein are methods of treating cancer in a subject by administering an effective amount of a multi-specific binding protein, or a pharmaceutical formulation thereof, once weekly in 4-week treatment cycles. The multi-specific binding protein incorporates: a first antigen binding site that binds NKG2D; a second antigen binding site that binds EGFR with (i) a VH having the CDR1, CDR2, and CDR3 sequences of SEQ ID NOs: 136, 157, and 138, respectively; and a VL having the CDR1, CDR2, and CDR3 sequences of SEQ ID NOs: 140, 141, and 151, respectively; (ii) a VH having the CDR1, CDR2, and CDR3 sequences of SEQ ID NOs: 136, 146, and 138, respectively; and a VL having the CDR1, CDR2, and CDR3 sequences of SEQ ID NOs: 140, 141, and 142, respectively; (iii) a VH having the CDR1, CDR2, and CDR3 sequences of SEQ ID NOs: 136, 146, and 138, respectively; and a VL having the CDR1, CDR2, and CDR3 sequences of SEQ ID NOs: 140, 141, and 151, respectively; (iv) a VH having the CDR1, CDR2, and CDR3 sequences of SEQ ID NOs: 136, 137, and 138, respectively; and a VL having the CDR1, CDR2, and CDR3 sequences of SEQ ID NOs: 140, 141, and 151, respectively; or (v) a VH having the CDR1, CDR2, and CDR3 sequences of SEQ ID NOs: 136, 137, and 138, respectively; and a VL having the CDR1, CDR2, and CDR3 sequences of SEQ ID NOs: 140, 141, and 142, respectively; and an antibody Fc domain or a portion thereof sufficient to bind CD16, or a third antigen-binding site that binds CD16.

In some embodiments, provided herein are methods of treating cancer in a subject by administering an effective amount of a multi-specific binding protein, or a pharmaceutical formulation thereof, once weekly in 4-week treatment cycles. The multi-specific binding protein incorporates: a first antigen binding site that binds NKG2D with a VH having the CDR1, CDR2, and CDR3 sequences of SEQ ID NOs: 81, 82, and 97 or 112, respectively; and a VL having the CDR1, CDR2, and CDR3 sequences of SEQ ID NOs: 86, 77, and 87, respectively; a second antigen binding site that binds EGFR with (i) a VH having the CDR1, CDR2, and CDR3 sequences of SEQ ID NOs: 136, 157, and 138, respectively; and a VL having the CDR1, CDR2, and CDR3 sequences of SEQ ID NOs: 140, 141, and 151, respectively; (ii) a VH having the CDR1, CDR2, and CDR3 sequences of SEQ ID NOs: 136, 146, and 138, respectively; and a VL having the CDR1, CDR2, and CDR3 sequences of SEQ ID NOs: 140, 141, and 142, respectively; (iii) a VH having the CDR1, CDR2, and CDR3 sequences of SEQ ID NOs: 136, 146, and 138, respectively; and a VL having the CDR1, CDR2, and CDR3 sequences of SEQ ID NOs: 140, 141, and 151, respectively; (iv) a VH having the CDR1, CDR2, and CDR3 sequences of SEQ ID NOs: 136, 137, and 138, respectively; and a VL having the CDR1, CDR2, and CDR3 sequences of SEQ ID NOs: 140, 141, and 151, respectively; or (v) a VH having the CDR1, CDR2, and CDR3 sequences of SEQ ID NOs: 136, 137, and 138, respectively; and a VL having the CDR1, CDR2, and CDR3 sequences of SEQ ID NOs: 140, 141, and 142, respectively; and an antibody Fc domain or a portion thereof sufficient to bind CD16, or a third antigen-binding site that binds CD16.

In some embodiments, provided herein are methods of treating cancer in a subject by administering an effective amount of a multi-specific binding protein, or a pharmaceutical formulation thereof, once weekly in 4-week treatment cycles. The multi-specific binding protein incorporates: a first polypeptide with the amino acid sequence of SEQ ID NO:167; (b) a second polypeptide with the amino acid sequence of SEQ ID NO:164; and (c) a third polypeptide with the amino acid sequence of SEQ ID NO:165.

In some embodiments, pharmaceutical formulations suitable for use in treating cancer in a subject, once weekly in 4-week treatment cycles, have 5 mg/mL to 50 mg/mL of the multi-specific binding protein (e.g., 5 mg/mL to 50 mg/mL, 5 mg/mL to 45 mg/mL, 5 mg/mL to 40 mg/mL, 5 mg/mL to 35 mg/mL, 5 mg/mL to 30 mg/mL, 5 mg/mL to 25 mg/mL, 5 mg/mL to 20 mg/mL, 5 mg/mL to 15 mg/mL, 5 mg/mL to 10 mg/mL, 10 mg/mL to 50 mg/mL, 15 mg/mL to 50 mg/mL, 20 mg/mL to 50 mg/mL, 25 mg/mL to 50 mg/mL, 30 mg/mL to 50 mg/mL, 35 mg/mL to 50 mg/mL, 40 mg/mL to 50 mg/mL, 45 mg/mL to 50 mg/mL, or 15 mg/mL), and also include: 15 mM to 25 mM citrate (e.g., 15 mM to 25 mM, 16 mM to 25 mM, 17 mM to 25 mM, 18 mM to 25 mM, 19 mM to 25 mM, 20 mM to 25 mM, 21 mM to 25 mM, 22 mM to 25 mM, 23 mM to 25 mM, 24 mM to 25 mM, 15 mM to 24 mM, 15 mM to 23 mM, 15 mM to 22 mM, 15 mM to 23 mM, 15 mM to 22 mM, 15 mM to 21 mM, 15 mM to 20 mM, 15 mM to 19 mM, 15 mM to 18 mM, 15 mM to 17 mM, 15 mM to 16 mM, or about 20 mM), 4% to 8% (w/v) mannitol (e.g., 4% to 8% (w/v), 4% to 7% (w/v), 4% to 6% (w/v), 4% to 5% (w/v), 5% to 8% (w/v), 6% to 8% (w/v), 7% to 8% (w/v), or about 6% (w/v)); and 0.005% to 0.05% (w/v) polysorbate 80 (e.g., 0.005% to 0.05% (w/v), 0.005% to 0.04% (w/v), 0.005% to 0.03% (w/v), 0.005% to 0.02% (w/v), 0.005% to 0.01% (w/v), 0.006% to 0.05% (w/v), 0.007% to 0.05% (w/v), 0.008% to 0.05% (w/v), 0.009% to 0.05% (w/v), 0.01% to 0.05% (w/v), 0.02% to 0.05% (w/v), 0.03% to 0.05% (w/v), 0.04% to 0.05% (w/v), or 0.01% (w/v)), at pH 6.2 to 6.8 (e.g., 6.2 to 6.8, 6.2 to 6.7, 6.2 to 6.6, 6.2 to 6.5, 6.2 to 6.4, 6.2 to 6.3, 6.3 to 6.8, 6.4 to 6.8, 6.5 to 6.8, 6.6 to 6.8, 6.7 to 6.8, or about 6.5). In some embodiments, pharmaceutical formulations suitable for use in treating cancer in a subject, once weekly in 4-week treatment cycles, include about 15 mg/mL of the multi-specific binding protein, and also include: about 20 mM citrate, about 6% (w/v) mannitol; and about 0.01% (w/v) polysorbate 80, at about pH 6.5.

In some embodiments, provided herein are methods of treating cancer in a subject-characterized as being eligible for anti-PD-1 or an anti-PD-L1 therapy for a malignancy of epithelial origin—by administering an anti-PD-1 or an anti-PD-L1 therapy in combination with an effective amount of a multi-specific binding protein, or a pharmaceutical formulation thereof. The multi-specific binding protein incorporates: a first antigen-binding site that binds NKG2D; a second antigen-binding site that binds EGFR; and an antibody Fc domain or a portion thereof sufficient to bind CD16, or a third antigen-binding site that binds CD16.

In some embodiments, provided herein are methods of treating cancer in a subject-characterized as being eligible for anti-PD-1 or an anti-PD-L1 therapy for a malignancy of epithelial origin—by administering an anti-PD-1 or an anti-PD-L1 therapy in combination with an effective amount of a multi-specific binding protein, or a pharmaceutical formulation thereof. The multi-specific binding protein incorporates: a first antigen binding site that binds NKG2D; a second antigen binding site that binds EGFR with (i) a VH having the CDR1, CDR2, and CDR3 sequences of SEQ ID NOs: 136, 157, and 138, respectively; and a VL having the CDR1, CDR2, and CDR3 sequences of SEQ ID NOs: 140, 141, and 151, respectively; (ii) a VH having the CDR1, CDR2, and CDR3 sequences of SEQ ID NOs: 136, 146, and 138, respectively; and a VL having the CDR1, CDR2, and CDR3 sequences of SEQ ID NOs: 140, 141, and 142, respectively; (iii) a VH having the CDR1, CDR2, and CDR3 sequences of SEQ ID NOs: 136, 146, and 138, respectively; and a VL having the CDR1, CDR2, and CDR3 sequences of SEQ ID NOs: 140, 141, and 151, respectively; (iv) a VH having the CDR1, CDR2, and CDR3 sequences of SEQ ID NOs: 136, 137, and 138, respectively; and a VL having the CDR1, CDR2, and CDR3 sequences of SEQ ID NOs: 140, 141, and 151, respectively; or (v) a VH having the CDR1, CDR2, and CDR3 sequences of SEQ ID NOs: 136, 137, and 138, respectively; and a VL having the CDR1, CDR2, and CDR3 sequences of SEQ ID NOs: 140, 141, and 142, respectively; and an antibody Fc domain or a portion thereof sufficient to bind CD16, or a third antigen-binding site that binds CD16.

In some embodiments, provided herein are methods of treating cancer in a subject-characterized as being eligible for anti-PD-1 or an anti-PD-L1 therapy for a malignancy of epithelial origin—by administering an anti-PD-1 or an anti-PD-L1 therapy in combination with an effective amount of a multi-specific binding protein, or a pharmaceutical formulation thereof. The multi-specific binding protein incorporates: a first antigen binding site that binds NKG2D with a VH having the CDR1, CDR2, and CDR3 sequences of SEQ ID NOs: 81, 82, and 97 or 112, respectively; and a VL having the CDR1, CDR2, and CDR3 sequences of SEQ ID NOs: 86, 77, and 87, respectively; a second antigen binding site that binds EGFR with (i) a VH having the CDR1, CDR2, and CDR3 sequences of SEQ ID NOs: 136, 157, and 138, respectively; and a VL having the CDR1, CDR2, and CDR3 sequences of SEQ ID NOs: 140, 141, and 151, respectively; (ii) a VH having the CDR1, CDR2, and CDR3 sequences of SEQ ID NOs: 136, 146, and 138, respectively; and a VL having the CDR1, CDR2, and CDR3 sequences of SEQ ID NOs: 140, 141, and 142, respectively; (iii) a VH having the CDR1, CDR2, and CDR3 sequences of SEQ ID NOs: 136, 146, and 138, respectively; and a VL having the CDR1, CDR2, and CDR3 sequences of SEQ ID NOs: 140, 141, and 151, respectively; (iv) a VH having the CDR1, CDR2, and CDR3 sequences of SEQ ID NOs: 136, 137, and 138, respectively; and a VL having the CDR1, CDR2, and CDR3 sequences of SEQ ID NOs: 140, 141, and 151, respectively; or (v) a VH having the CDR1, CDR2, and CDR3 sequences of SEQ ID NOs: 136, 137, and 138, respectively; and a VL having the CDR1, CDR2, and CDR3 sequences of SEQ ID NOs: 140, 141, and 142, respectively; and an antibody Fc domain or a portion thereof sufficient to bind CD16, or a third antigen-binding site that binds CD16.

In some embodiments, provided herein are methods of treating cancer in a subject-characterized as being eligible for anti-PD-1 or an anti-PD-L1 therapy for a malignancy of epithelial origin—by administering an anti-PD-1 or an anti-PD-L1 therapy in combination with an effective amount of a multi-specific binding protein, or a pharmaceutical formulation thereof. The multi-specific binding protein incorporates: a first polypeptide with the amino acid sequence of SEQ ID NO:167; (b) a second polypeptide with the amino acid sequence of SEQ ID NO:164; and (c) a third polypeptide with the amino acid sequence of SEQ ID NO:165.

In some embodiments, pharmaceutical formulations-suitable for use in combination with an anti-PD-1 or an anti-PD-L1 therapy for treating cancer in a subject characterized as being eligible for anti-PD-1 or an anti-PD-L1 therapy for a malignancy of epithelial origin—have 5 mg/mL to 50 mg/mL of the multi-specific binding protein (e.g., 5 mg/mL to 50 mg/mL, 5 mg/mL to 45 mg/mL, 5 mg/mL to 40 mg/mL, 5 mg/mL to 35 mg/mL, 5 mg/mL to 30 mg/mL, 5 mg/mL to 25 mg/mL, 5 mg/mL to 20 mg/mL, 5 mg/mL to 15 mg/mL, 5 mg/mL to 10 mg/mL, 10 mg/mL to 50 mg/mL, 15 mg/mL to 50 mg/mL, 20 mg/mL to 50 mg/mL, 25 mg/mL to 50 mg/mL, 30 mg/mL to 50 mg/mL, 35 mg/mL to 50 mg/mL, 40 mg/mL to 50 mg/mL, 45 mg/mL to 50 mg/mL, or 15 mg/mL), and also include: 15 mM to 25 mM citrate (e.g., 15 mM to 25 mM, 16 mM to 25 mM, 17 mM to 25 mM, 18 mM to 25 mM, 19 mM to 25 mM, 20 mM to 25 mM, 21 mM to 25 mM, 22 mM to 25 mM, 23 mM to 25 mM, 24 mM to 25 mM, 15 mM to 24 mM, 15 mM to 23 mM, 15 mM to 22 mM, 15 mM to 23 mM, 15 mM to 22 mM, 15 mM to 21 mM, 15 mM to 20 mM, 15 mM to 19 mM, 15 mM to 18 mM, 15 mM to 17 mM, 15 mM to 16 mM, or about 20 mM), 4% to 8% (w/v) mannitol (e.g., 4% to 8% (w/v), 4% to 7% (w/v), 4% to 6% (w/v), 4% to 5% (w/v), 5% to 8% (w/v), 6% to 8% (w/v), 7% to 8% (w/v), or about 6% (w/v)); and 0.005% to 0.05% (w/v) polysorbate 80 (e.g., 0.005% to 0.05% (w/v), 0.005% to 0.04% (w/v), 0.005% to 0.03% (w/v), 0.005% to 0.02% (w/v), 0.005% to 0.01% (w/v), 0.006% to 0.05% (w/v), 0.007% to 0.05% (w/v), 0.008% to 0.05% (w/v), 0.009% to 0.05% (w/v), 0.01% to 0.05% (w/v), 0.02% to 0.05% (w/v), 0.03% to 0.05% (w/v), 0.04% to 0.05% (w/v), or 0.01% (w/v)), at pH 6.2 to 6.8 (e.g., 6.2 to 6.8, 6.2 to 6.7, 6.2 to 6.6, 6.2 to 6.5, 6.2 to 6.4, 6.2 to 6.3, 6.3 to 6.8, 6.4 to 6.8, 6.5 to 6.8, 6.6 to 6.8, 6.7 to 6.8, or about 6.5). In some embodiments, pharmaceutical formulations-suitable for use in combination with an anti-PD-1 or an anti-PD-L1 therapy for treating cancer in a subject characterized as being eligible for anti-PD-1 or an anti-PD-L1 therapy for a malignancy of epithelial origin-include about 15 mg/mL of the multi-specific binding protein, and also include: about 20 mM citrate, about 6% (w/v) mannitol; and about 0.01% (w/v) polysorbate 80, at about pH 6.5.

In some embodiments, provided herein are methods of treating cancer in a subject—for which no standard therapy exists or, even when a standard therapy exists, the standard therapy of the subject has failed for a malignancy of epithelial origin—by administering an anti-PD-1 or an anti-PD-L1 therapy in combination with an effective amount of a multi-specific binding protein, or a pharmaceutical formulation thereof. The multi-specific binding protein incorporates: a first antigen-binding site that binds NKG2D; a second antigen-binding site that binds EGFR; and an antibody Fc domain or a portion thereof sufficient to bind CD16, or a third antigen-binding site that binds CD16.

In some embodiments, provided herein are methods of treating cancer in a subject—for which no standard therapy exists or, even when a standard therapy exists, the standard therapy of the subject has failed for a malignancy of epithelial origin—by administering an anti-PD-1 or an anti-PD-L1 therapy in combination with an effective amount of a multi-specific binding protein, or a pharmaceutical formulation thereof. The multi-specific binding protein incorporates: a first antigen binding site that binds NKG2D; a second antigen binding site that binds EGFR with (i) a VH having the CDR1, CDR2, and CDR3 sequences of SEQ ID NOs: 136, 157, and 138, respectively; and a VL having the CDR1, CDR2, and CDR3 sequences of SEQ ID NOs: 140, 141, and 151, respectively; (ii) a VH having the CDR1, CDR2, and CDR3 sequences of SEQ ID NOs: 136, 146, and 138, respectively; and a VL having the CDR1, CDR2, and CDR3 sequences of SEQ ID NOs: 140, 141, and 142, respectively; (iii) a VH having the CDR1, CDR2, and CDR3 sequences of SEQ ID NOs: 136, 146, and 138, respectively; and a VL having the CDR1, CDR2, and CDR3 sequences of SEQ ID NOs: 140, 141, and 151, respectively; (iv) a VH having the CDR1, CDR2, and CDR3 sequences of SEQ ID NOs: 136, 137, and 138, respectively; and a VL having the CDR1, CDR2, and CDR3 sequences of SEQ ID NOs: 140, 141, and 151, respectively; or (v) a VH having the CDR1, CDR2, and CDR3 sequences of SEQ ID NOs: 136, 137, and 138, respectively; and a VL having the CDR1, CDR2, and CDR3 sequences of SEQ ID NOs: 140, 141, and 142, respectively; and an antibody Fc domain or a portion thereof sufficient to bind CD16, or a third antigen-binding site that binds CD16.

In some embodiments, provided herein are methods of treating cancer in a subject—for which no standard therapy exists or, even when a standard therapy exists, the standard therapy of the subject has failed for a malignancy of epithelial origin—by administering an anti-PD-1 or an anti-PD-L1 therapy in combination with an effective amount of a multi-specific binding protein, or a pharmaceutical formulation thereof. The multi-specific binding protein incorporates: a first antigen binding site that binds NKG2D with a VH having the CDR1, CDR2, and CDR3 sequences of SEQ ID NOs: 81, 82, and 97 or 112, respectively; and a VL having the CDR1, CDR2, and CDR3 sequences of SEQ ID NOs: 86, 77, and 87, respectively; a second antigen binding site that binds EGFR with (i) a VH having the CDR1, CDR2, and CDR3 sequences of SEQ ID NOs: 136, 157, and 138, respectively; and a VL having the CDR1, CDR2, and CDR3 sequences of SEQ ID NOs: 140, 141, and 151, respectively; (ii) a VH having the CDR1, CDR2, and CDR3 sequences of SEQ ID NOs: 136, 146, and 138, respectively; and a VL having the CDR1, CDR2, and CDR3 sequences of SEQ ID NOs: 140, 141, and 142, respectively; (iii) a VH having the CDR1, CDR2, and CDR3 sequences of SEQ ID NOs: 136, 146, and 138, respectively; and a VL having the CDR1, CDR2, and CDR3 sequences of SEQ ID NOs: 140, 141, and 151, respectively; (iv) a VH having the CDR1, CDR2, and CDR3 sequences of SEQ ID NOs: 136, 137, and 138, respectively; and a VL having the CDR1, CDR2, and CDR3 sequences of SEQ ID NOs: 140, 141, and 151, respectively; or (v) a VH having the CDR1, CDR2, and CDR3 sequences of SEQ ID NOs: 136, 137, and 138, respectively; and a VL having the CDR1, CDR2, and CDR3 sequences of SEQ ID NOs: 140, 141, and 142, respectively; and an antibody Fc domain or a portion thereof sufficient to bind CD16, or a third antigen-binding site that binds CD16.

In some embodiments, provided herein are methods of treating cancer in a subject—for which no standard therapy exists or, even when a standard therapy exists, the standard therapy of the subject has failed for a malignancy of epithelial origin—by administering an anti-PD-1 or an anti-PD-L1 therapy in combination with an effective amount of a multi-specific binding protein, or a pharmaceutical formulation thereof. The multi-specific binding protein incorporates: a first polypeptide with the amino acid sequence of SEQ ID NO:167; (b) a second polypeptide with the amino acid sequence of SEQ ID NO:164; and (c) a third polypeptide with the amino acid sequence of SEQ ID NO:165.

In some embodiments, pharmaceutical formulations suitable for use in treating cancer in a subject—for which no standard therapy exists or, even when a standard therapy exists, the standard therapy of the subject has failed for a malignancy of epithelial origin—in combination with an anti-PD-1 or an anti-PD-L1 therapy, have 5 mg/mL to 50 mg/mL of the multi-specific binding protein (e.g., 5 mg/mL to 50 mg/mL, 5 mg/mL to 45 mg/mL, 5 mg/mL to 40 mg/mL, 5 mg/mL to 35 mg/mL, 5 mg/mL to 30 mg/mL, 5 mg/mL to 25 mg/mL, 5 mg/mL to 20 mg/mL, 5 mg/mL to 15 mg/mL, 5 mg/mL to 10 mg/mL, 10 mg/mL to 50 mg/mL, 15 mg/mL to 50 mg/mL, 20 mg/mL to 50 mg/mL, 25 mg/mL to 50 mg/mL, 30 mg/mL to 50 mg/mL, 35 mg/mL to 50 mg/mL, 40 mg/mL to 50 mg/mL, 45 mg/mL to 50 mg/mL, or 15 mg/mL), and also include: 15 mM to 25 mM citrate (e.g., 15 mM to 25 mM, 16 mM to 25 mM, 17 mM to 25 mM, 18 mM to 25 mM, 19 mM to 25 mM, 20 mM to 25 mM, 21 mM to 25 mM, 22 mM to 25 mM, 23 mM to 25 mM, 24 mM to 25 mM, 15 mM to 24 mM, 15 mM to 23 mM, 15 mM to 22 mM, 15 mM to 23 mM, 15 mM to 22 mM, 15 mM to 21 mM, 15 mM to 20 mM, 15 mM to 19 mM, 15 mM to 18 mM, 15 mM to 17 mM, 15 mM to 16 mM, or about 20 mM), 4% to 8% (w/v) mannitol (e.g., 4% to 8% (w/v), 4% to 7% (w/v), 4% to 6% (w/v), 4% to 5% (w/v), 5% to 8% (w/v), 6% to 8% (w/v), 7% to 8% (w/v), or about 6% (w/v)); and 0.005% to 0.05% (w/v) polysorbate 80 (e.g., 0.005% to 0.05% (w/v), 0.005% to 0.04% (w/v), 0.005% to 0.03% (w/v), 0.005% to 0.02% (w/v), 0.005% to 0.01% (w/v), 0.006% to 0.05% (w/v), 0.007% to 0.05% (w/v), 0.008% to 0.05% (w/v), 0.009% to 0.05% (w/v), 0.01% to 0.05% (w/v), 0.02% to 0.05% (w/v), 0.03% to 0.05% (w/v), 0.04% to 0.05% (w/v), or 0.01% (w/v)), at pH 6.2 to 6.8 (e.g., 6.2 to 6.8, 6.2 to 6.7, 6.2 to 6.6, 6.2 to 6.5, 6.2 to 6.4, 6.2 to 6.3, 6.3 to 6.8, 6.4 to 6.8, 6.5 to 6.8, 6.6 to 6.8, 6.7 to 6.8, or about 6.5). In some embodiments, pharmaceutical formulations suitable for use in treating cancer in a subject—for which no standard therapy exists or, even when a standard therapy exists, the standard therapy of the subject has failed for a malignancy of epithelial origin—in combination with an anti-PD-1 or an anti-PD-L1 therapy, include about 15 mg/mL of the multi-specific binding protein, and also include: about 20 mM citrate, about 6% (w/v) mannitol; and about 0.01% (w/v) polysorbate 80, at about pH 6.5.

In some embodiments, provided herein are methods of treating cancer in a subject, characterized as having previously received an anti-PD-1 or anti-PD-L1 therapy, by administering an anti-PD-1 or an anti-PD-L1 therapy in combination with an effective amount of a multi-specific binding protein, or a pharmaceutical formulation thereof. The multi-specific binding protein incorporates: a first antigen-binding site that binds NKG2D; a second antigen-binding site that binds EGFR; and an antibody Fc domain or a portion thereof sufficient to bind CD16, or a third antigen-binding site that binds CD16.

In some embodiments, provided herein are methods of treating cancer in a subject, characterized as having previously received an anti-PD-1 or anti-PD-L1 therapy, by administering an anti-PD-1 or an anti-PD-L1 therapy in combination with an effective amount of a multi-specific binding protein, or a pharmaceutical formulation thereof. The multi-specific binding protein incorporates: a first antigen binding site that binds NKG2D; a second antigen binding site that binds EGFR with (i) a VH having the CDR1, CDR2, and CDR3 sequences of SEQ ID NOs: 136, 157, and 138, respectively; and a VL having the CDR1, CDR2, and CDR3 sequences of SEQ ID NOs: 140, 141, and 151, respectively; (ii) a VH having the CDR1, CDR2, and CDR3 sequences of SEQ ID NOs: 136, 146, and 138, respectively; and a VL having the CDR1, CDR2, and CDR3 sequences of SEQ ID NOs: 140, 141, and 142, respectively; (iii) a VH having the CDR1, CDR2, and CDR3 sequences of SEQ ID NOs: 136, 146, and 138, respectively; and a VL having the CDR1, CDR2, and CDR3 sequences of SEQ ID NOs: 140, 141, and 151, respectively; (iv) a VH having the CDR1, CDR2, and CDR3 sequences of SEQ ID NOs: 136, 137, and 138, respectively; and a VL having the CDR1, CDR2, and CDR3 sequences of SEQ ID NOs: 140, 141, and 151, respectively; or (v) a VH having the CDR1, CDR2, and CDR3 sequences of SEQ ID NOs: 136, 137, and 138, respectively; and a VL having the CDR1, CDR2, and CDR3 sequences of SEQ ID NOs: 140, 141, and 142, respectively; and an antibody Fc domain or a portion thereof sufficient to bind CD16, or a third antigen-binding site that binds CD16.

In some embodiments, provided herein are methods of treating cancer in a subject, characterized as having previously received an anti-PD-1 or anti-PD-L1 therapy, by administering an anti-PD-1 or an anti-PD-L1 therapy in combination with an effective amount of a multi-specific binding protein, or a pharmaceutical formulation thereof. The multi-specific binding protein incorporates: a first antigen binding site that binds NKG2D with a VH having the CDR1, CDR2, and CDR3 sequences of SEQ ID NOs: 81, 82, and 97 or 112, respectively; and a VL having the CDR1, CDR2, and CDR3 sequences of SEQ ID NOs: 86, 77, and 87, respectively; a second antigen binding site that binds EGFR with (i) a VH having the CDR1, CDR2, and CDR3 sequences of SEQ ID NOs: 136, 157, and 138, respectively; and a VL having the CDR1, CDR2, and CDR3 sequences of SEQ ID NOs: 140, 141, and 151, respectively; (ii) a VH having the CDR1, CDR2, and CDR3 sequences of SEQ ID NOs: 136, 146, and 138, respectively; and a VL having the CDR1, CDR2, and CDR3 sequences of SEQ ID NOs: 140, 141, and 142, respectively; (iii) a VH having the CDR1, CDR2, and CDR3 sequences of SEQ ID NOs: 136, 146, and 138, respectively; and a VL having the CDR1, CDR2, and CDR3 sequences of SEQ ID NOs: 140, 141, and 151, respectively; (iv) a VH having the CDR1, CDR2, and CDR3 sequences of SEQ ID NOs: 136, 137, and 138, respectively; and a VL having the CDR1, CDR2, and CDR3 sequences of SEQ ID NOs: 140, 141, and 151, respectively; or (v) a VH having the CDR1, CDR2, and CDR3 sequences of SEQ ID NOs: 136, 137, and 138, respectively; and a VL having the CDR1, CDR2, and CDR3 sequences of SEQ ID NOs: 140, 141, and 142, respectively; and an antibody Fc domain or a portion thereof sufficient to bind CD16, or a third antigen-binding site that binds CD16.

In some embodiments, provided herein are methods of treating cancer in a subject, characterized as having previously received an anti-PD-1 or anti-PD-L1 therapy, by administering an anti-PD-1 or an anti-PD-L1 therapy in combination with an effective amount of a multi-specific binding protein, or a pharmaceutical formulation thereof. The multi-specific binding protein incorporates: a first polypeptide with the amino acid sequence of SEQ ID NO:167; (b) a second polypeptide with the amino acid sequence of SEQ ID NO:164; and (c) a third polypeptide with the amino acid sequence of SEQ ID NO:165.

In some embodiments, pharmaceutical formulations, suitable for use in combination with an anti-PD-1 or an anti-PD-L1 therapy for treating cancer in a subject characterized as having previously received an anti-PD-1 or anti-PD-L1 therapy, have 5 mg/mL to 50 mg/mL of the multi-specific binding protein (e.g., 5 mg/mL to 50 mg/mL, 5 mg/mL to 45 mg/mL, 5 mg/mL to 40 mg/mL, 5 mg/mL to 35 mg/mL, 5 mg/mL to 30 mg/mL, 5 mg/mL to 25 mg/mL, 5 mg/mL to 20 mg/mL, 5 mg/mL to 15 mg/mL, 5 mg/mL to 10 mg/mL, 10 mg/mL to 50 mg/mL, 15 mg/mL to 50 mg/mL, 20 mg/mL to 50 mg/mL, 25 mg/mL to 50 mg/mL, 30 mg/mL to 50 mg/mL, 35 mg/mL to 50 mg/mL, 40 mg/mL to 50 mg/mL, 45 mg/mL to 50 mg/mL, or 15 mg/mL), and also include: 15 mM to 25 mM citrate (e.g., 15 mM to 25 mM, 16 mM to 25 mM, 17 mM to 25 mM, 18 mM to 25 mM, 19 mM to 25 mM, 20 mM to 25 mM, 21 mM to 25 mM, 22 mM to 25 mM, 23 mM to 25 mM, 24 mM to 25 mM, 15 mM to 24 mM, 15 mM to 23 mM, 15 mM to 22 mM, 15 mM to 23 mM, 15 mM to 22 mM, 15 mM to 21 mM, 15 mM to 20 mM, 15 mM to 19 mM, 15 mM to 18 mM, 15 mM to 17 mM, 15 mM to 16 mM, or about 20 mM), 4% to 8% (w/v) mannitol (e.g., 4% to 8% (w/v), 4% to 7% (w/v), 4% to 6% (w/v), 4% to 5% (w/v), 5% to 8% (w/v), 6% to 8% (w/v), 7% to 8% (w/v), or about 6% (w/v)); and 0.005% to 0.05% (w/v) polysorbate 80 (e.g., 0.005% to 0.05% (w/v), 0.005% to 0.04% (w/v), 0.005% to 0.03% (w/v), 0.005% to 0.02% (w/v), 0.005% to 0.01% (w/v), 0.006% to 0.05% (w/v), 0.007% to 0.05% (w/v), 0.008% to 0.05% (w/v), 0.009% to 0.05% (w/v), 0.01% to 0.05% (w/v), 0.02% to 0.05% (w/v), 0.03% to 0.05% (w/v), 0.04% to 0.05% (w/v), or 0.01% (w/v)), at pH 6.2 to 6.8 (e.g., 6.2 to 6.8, 6.2 to 6.7, 6.2 to 6.6, 6.2 to 6.5, 6.2 to 6.4, 6.2 to 6.3, 6.3 to 6.8, 6.4 to 6.8, 6.5 to 6.8, 6.6 to 6.8, 6.7 to 6.8, or about 6.5). In some embodiments, pharmaceutical formulations, suitable for use in combination with an anti-PD-1 or an anti-PD-L1 therapy for treating cancer in a subject characterized as having previously received an anti-PD-1 or anti-PD-L1 therapy, include about 15 mg/mL of the multi-specific binding protein, and also include: about 20 mM citrate, about 6% (w/v) mannitol; and about 0.01% (w/v) polysorbate 80, at about pH 6.5.

In some embodiments, provided herein are methods for treating cancer in a subject, where the subject has experienced either:
a grade 3 or 4 drug-related toxicity during and attributed to treatment with the anti-PD-1 or anti-PD-L1 therapy; or
a grade 2 drug-related toxicity attributed to the use of an anti-PD-1 or an anti-PD-L1 therapy that impacted either the lungs or the nervous system.

In some embodiments, provided herein are methods of treating head and neck squamous cell carcinoma (HNSCC) in a subject by administering an effective amount of a multi-specific binding protein, or a pharmaceutical formulation thereof. The multi-specific binding protein incorporates: a first antigen-binding site that binds NKG2D; a second antigen-binding site that binds EGFR; and an antibody Fc domain or a portion thereof sufficient to bind CD16, or a third antigen-binding site that binds CD16. In some embodiments, the subject treated has relapsed or metastatic HNSCC.

In some embodiments, provided herein are methods of treating HNSCC in a subject by administering an effective amount of a multi-specific binding protein, or a pharmaceutical formulation thereof. The multi-specific binding protein incorporates: a first antigen binding site that binds NKG2D; a second antigen binding site that binds EGFR with (i) a VH having the CDR1, CDR2, and CDR3 sequences of SEQ ID NOs: 136, 157, and 138, respectively; and a VL having the CDR1, CDR2, and CDR3 sequences of SEQ ID NOs: 140, 141, and 151, respectively; (ii) a VH having the CDR1, CDR2, and CDR3 sequences of SEQ ID NOs: 136, 146, and 138, respectively; and a VL having the CDR1, CDR2, and CDR3 sequences of SEQ ID NOs: 140, 141, and 142, respectively; (iii) a VH having the CDR1, CDR2, and CDR3 sequences of SEQ ID NOs: 136, 146, and 138, respectively; and a VL having the CDR1, CDR2, and CDR3 sequences of SEQ ID NOs: 140, 141, and 151, respectively; (iv) a VH having the CDR1, CDR2, and CDR3 sequences of SEQ ID NOs: 136, 137, and 138, respectively; and a VL having the CDR1, CDR2, and CDR3 sequences of SEQ ID NOs: 140, 141, and 151, respectively; or (v) a VH having the CDR1, CDR2, and CDR3 sequences of SEQ ID NOs: 136, 137, and 138, respectively; and a VL having the CDR1, CDR2, and CDR3 sequences of SEQ ID NOs: 140, 141, and 142, respectively; and an antibody Fc domain or a portion thereof sufficient to bind CD16, or a third antigen-binding site that binds CD16. In some embodiments, the subject treated has relapsed or metastatic HNSCC.

In some embodiments, provided herein are methods of treating HNSCC in a subject by administering an effective amount of a multi-specific binding protein, or a pharmaceutical formulation thereof. The multi-specific binding protein incorporates: a first antigen binding site that binds NKG2D with a VH having the CDR1, CDR2, and CDR3 sequences of SEQ ID NOs: 81, 82, and 97 or 112, respectively; and a VL having the CDR1, CDR2, and CDR3 sequences of SEQ ID NOs: 86, 77, and 87, respectively; a second antigen binding site that binds EGFR including (i) a VH having the CDR1, CDR2, and CDR3 sequences of SEQ ID NOs: 136, 157, and 138, respectively; and a VL having the CDR1, CDR2, and CDR3 sequences of SEQ ID NOs: 140, 141, and 151, respectively; (ii) a VH having the CDR1, CDR2, and CDR3 sequences of SEQ ID NOs: 136, 146, and 138, respectively; and a VL having the CDR1, CDR2, and CDR3 sequences of SEQ ID NOs: 140, 141, and 142, respectively; (iii) a VH having the CDR1, CDR2, and CDR3 sequences of SEQ ID NOs: 136, 146, and 138, respectively; and a VL having the CDR1, CDR2, and CDR3 sequences of SEQ ID NOs: 140, 141, and 151, respectively; (iv) a VH having the CDR1, CDR2, and CDR3 sequences of SEQ ID NOs: 136, 137, and 138, respectively; and a VL having the CDR1, CDR2, and CDR3 sequences of SEQ ID NOs: 140, 141, and 151, respectively; or (v) a VH having the CDR1, CDR2, and CDR3 sequences of SEQ ID NOs: 136, 137, and 138, respectively; and a VL having the CDR1, CDR2, and CDR3 sequences of SEQ ID NOs: 140, 141, and 142, respectively; and an antibody Fc domain or a portion thereof sufficient to bind CD16, or a third antigen-binding site that binds CD16. In some embodiments, the subject treated has relapsed or metastatic HNSCC.

In some embodiments, provided herein are methods of treating HNSCC in a subject by administering an effective amount of a multi-specific binding protein, or a pharmaceutical formulation thereof. The multi-specific binding protein incorporates: a first polypeptide with the amino acid sequence of SEQ ID NO:167; (b) a second polypeptide with the amino acid sequence of SEQ ID NO:164; and (c) a third polypeptide with the amino acid sequence of SEQ ID NO:165. In some embodiments, the subject treated has relapsed or metatstatic HNSCC.

In some embodiments, pharmaceutical formulations suitable for use in treating relapsed or metastatic HNSCC in a subject have 5 mg/mL to 50 mg/mL of the multi-specific binding protein (e.g., 5 mg/mL to 50 mg/mL, 5 mg/mL to 45 mg/mL, 5 mg/mL to 40 mg/mL, 5 mg/mL to 35 mg/mL, 5 mg/mL to 30 mg/mL, 5 mg/mL to 25 mg/mL, 5 mg/mL to 20 mg/mL, 5 mg/mL to 15 mg/mL, 5 mg/mL to 10 mg/mL, 10 mg/mL to 50 mg/mL, 15 mg/mL to 50 mg/mL, 20 mg/mL to 50 mg/mL, 25 mg/mL to 50 mg/mL, 30 mg/mL to 50 mg/mL, 35 mg/mL to 50 mg/mL, 40 mg/mL to 50 mg/mL, 45 mg/mL to 50 mg/mL, or 15 mg/mL), and also include: 15 mM to 25 mM citrate (e.g., 15 mM to 25 mM, 16 mM to 25 mM, 17 mM to 25 mM, 18 mM to 25 mM, 19 mM to 25 mM, 20 mM to 25 mM, 21 mM to 25 mM, 22 mM to 25 mM, 23 mM to 25 mM, 24 mM to 25 mM, 15 mM to 24 mM, 15 mM to 23 mM, 15 mM to 22 mM, 15 mM to 23 mM, 15 mM to 22 mM, 15 mM to 21 mM, 15 mM to 20 mM, 15 mM to 19 mM, 15 mM to 18 mM, 15 mM to 17 mM, 15 mM to 16 mM, or about 20 mM), 4% to 8% (w/v) mannitol (e.g., 4% to 8% (w/v), 4% to 7% (w/v), 4% to 6% (w/v), 4% to 5% (w/v), 5% to 8% (w/v), 6% to 8% (w/v), 7% to 8% (w/v), or about 6% (w/v)); and 0.005% to 0.05% (w/v) polysorbate 80 (e.g., 0.005% to 0.05% (w/v), 0.005% to 0.04% (w/v), 0.005% to 0.03% (w/v), 0.005% to 0.02% (w/v), 0.005% to 0.01% (w/v), 0.006% to 0.05% (w/v), 0.007% to 0.05% (w/v), 0.008% to 0.05% (w/v), 0.009% to 0.05% (w/v), 0.01% to 0.05% (w/v), 0.02% to 0.05% (w/v), 0.03% to 0.05% (w/v), 0.04% to 0.05% (w/v), or 0.01% (w/v)), at pH 6.2 to 6.8 (e.g., 6.2 to 6.8, 6.2 to 6.7, 6.2 to 6.6, 6.2 to 6.5, 6.2 to 6.4, 6.2 to 6.3, 6.3 to 6.8, 6.4 to 6.8, 6.5 to 6.8, 6.6 to 6.8, 6.7 to 6.8, or about 6.5). In some embodiments, pharmaceutical formulations suitable for use in treating HNSCC in a subject include about 15 mg/mL of the multi-specific binding protein, and also include: about 20 mM citrate, about 6% (w/v) mannitol; and about 0.01% (w/v) polysorbate 80, at about pH 6.5. In some embodiments, pharmaceutical formulations suitable for use in treating relapsed or metastatic HNSCC in a subject include about 15 mg/mL of the multi-specific binding protein, and also include: about 20 mM citrate, about 6% (w/v) mannitol; and about 0.01% (w/v) polysorbate 80, at about pH 6.5.

In some embodiments, provided herein are methods of treating head and neck squamous cell carcinoma (HNSCC) in a subject characterized as having radiographic disease progression while on or after having received: (i) pembrolizumab and platinum/5FU, (ii) pembrolizumab monotherapy, or (iii) platinum/5FU and cetuximab, by administering an effective amount of a multi-specific binding protein, or a pharmaceutical formulation thereof. The multi-specific binding protein incorporates: a first antigen-binding site that binds NKG2D; a second antigen-binding site that binds EGFR; and an antibody Fc domain or a portion thereof sufficient to bind CD16, or a third antigen-binding site that binds CD16.

In some embodiments, provided herein are methods of treating HNSCC in a subject characterized as having radiographic disease progression while on or after having received: (i) pembrolizumab and platinum/5FU, (ii) pembrolizumab monotherapy, or (iii) platinum/5FU and cetuximab, by administering an effective amount of a multi-specific binding protein, or a pharmaceutical formulation thereof. The multi-specific binding protein incorporates: a first antigen binding site that binds NKG2D; a second antigen binding site that binds EGFR with (i) a VH having the CDR1, CDR2, and CDR3 sequences of SEQ ID NOs: 136, 157, and 138, respectively; and a VL having the CDR1, CDR2, and CDR3 sequences of SEQ ID NOs: 140, 141, and 151, respectively; (ii) a VH having the CDR1, CDR2, and CDR3 sequences of SEQ ID NOs: 136, 146, and 138, respectively; and a VL having the CDR1, CDR2, and CDR3 sequences of SEQ ID NOs: 140, 141, and 142, respectively; (iii) a VH having the CDR1, CDR2, and CDR3 sequences of SEQ ID NOs: 136, 146, and 138, respectively; and a VL having the CDR1, CDR2, and CDR3 sequences of SEQ ID NOs: 140, 141, and 151, respectively; (iv) a VH having the CDR1, CDR2, and CDR3 sequences of SEQ ID NOs: 136, 137, and 138, respectively; and a VL having the CDR1, CDR2, and CDR3 sequences of SEQ ID NOs: 140, 141, and 151, respectively; or (v) a VH having the CDR1, CDR2, and CDR3 sequences of SEQ ID NOs: 136, 137, and 138, respectively; and a VL having the CDR1, CDR2, and CDR3 sequences of SEQ ID NOs: 140, 141, and 142, respectively; and an antibody Fc domain or a portion thereof sufficient to bind CD16, or a third antigen-binding site that binds CD16.

In some embodiments, provided herein are methods of treating HNSCC in a subject characterized as having radiographic disease progression while on or after having received: (i) pembrolizumab and platinum/5FU, (ii) pembrolizumab monotherapy, or (iii) platinum/5FU and cetuximab, by administering an effective amount of a multi-specific binding protein, or a pharmaceutical formulation thereof. The multi-specific binding protein incorporates: a first antigen binding site that binds NKG2D with a VH having the CDR1, CDR2, and CDR3 sequences of SEQ ID NOs: 81, 82, and 97 or 112, respectively; and a VL having the CDR1, CDR2, and CDR3 sequences of SEQ ID NOs: 86, 77, and 87, respectively; a second antigen binding site that binds EGFR with (i) a VH having the CDR1, CDR2, and CDR3 sequences of SEQ ID NOs: 136, 157, and 138, respectively; and a VL having the CDR1, CDR2, and CDR3 sequences of SEQ ID NOs: 140, 141, and 151, respectively; (ii) a VH having the CDR1, CDR2, and CDR3 sequences of SEQ ID NOs: 136, 146, and 138, respectively; and a VL having the CDR1, CDR2, and CDR3 sequences of SEQ ID NOs: 140, 141, and 142, respectively; (iii) a VH having the CDR1, CDR2, and CDR3 sequences of SEQ ID NOs: 136, 146, and 138, respectively; and a VL having the CDR1, CDR2, and CDR3 sequences of SEQ ID NOs: 140, 141, and 151, respectively; (iv) a VH having the CDR1, CDR2, and CDR3 sequences of SEQ ID NOs: 136, 137, and 138, respectively; and a VL having the CDR1, CDR2, and CDR3 sequences of SEQ ID NOs: 140, 141, and 151, respectively; or (v) a VH having the CDR1, CDR2, and CDR3 sequences of SEQ ID NOs: 136, 137, and 138, respectively; and a VL having the CDR1, CDR2, and CDR3 sequences of SEQ ID NOs: 140, 141, and 142, respectively; and an antibody Fc domain or a portion thereof sufficient to bind CD16, or a third antigen-binding site that binds CD16.

In some embodiments, provided herein are methods of treating HNSCC in a subject characterized as having radiographic disease progression while on or after having received: (i) pembrolizumab and platinum/5FU, (ii) pembrolizumab monotherapy, or (iii) platinum/5FU and cetuximab, by administering an effective amount of a multi-specific binding protein, or a pharmaceutical formulation thereof. The multi-specific binding protein incorporates: a first polypeptide with the amino acid sequence of SEQ ID NO:167; (b) a second polypeptide with the amino acid sequence of SEQ ID NO:164; and (c) a third polypeptide with the amino acid sequence of SEQ ID NO:165.

In some embodiments, pharmaceutical formulations suitable for use in treating HNSCC in a subject characterized as having radiographic disease progression while on or after having received: (i) pembrolizumab and platinum/5FU, (ii) pembrolizumab monotherapy, or (iii) platinum/5FU and cetuximab, have 5 mg/mL to 50 mg/mL of the multi-specific binding protein (e.g., 5 mg/mL to 50 mg/mL, 5 mg/mL to 45 mg/mL, 5 mg/mL to 40 mg/mL, 5 mg/mL to 35 mg/mL, 5 mg/mL to 30 mg/mL, 5 mg/mL to 25 mg/mL, 5 mg/mL to 20 mg/mL, 5 mg/mL to 15 mg/mL, 5 mg/mL to 10 mg/mL, 10 mg/mL to 50 mg/mL, 15 mg/mL to 50 mg/mL, 20 mg/mL to 50 mg/mL, 25 mg/mL to 50 mg/mL, 30 mg/mL to 50 mg/mL, 35 mg/mL to 50 mg/mL, 40 mg/mL to 50 mg/mL, 45 mg/mL to 50 mg/mL, or 15 mg/mL), and also include: 15 mM to 25 mM citrate (e.g., 15 mM to 25 mM, 16 mM to 25 mM, 17 mM to 25 mM, 18 mM to 25 mM, 19 mM to 25 mM, 20 mM to 25 mM, 21 mM to 25 mM, 22 mM to 25 mM, 23 mM to 25 mM, 24 mM to 25 mM, 15 mM to 24 mM, 15 mM to 23 mM, 15 mM to 22 mM, 15 mM to 23 mM, 15 mM to 22 mM, 15 mM to 21 mM, 15 mM to 20 mM, 15 mM to 19 mM, 15 mM to 18 mM, 15 mM to 17 mM, 15 mM to 16 mM, or about 20 mM), 4% to 8% (w/v) mannitol (e.g., 4% to 8% (w/v), 4% to 7% (w/v), 4% to 6% (w/v), 4% to 5% (w/v), 5% to 8% (w/v), 6% to 8% (w/v), 7% to 8% (w/v), or about 6% (w/v)); and 0.005% to 0.05% (w/v) polysorbate 80 (e.g., 0.005% to 0.05% (w/v), 0.005% to 0.04% (w/v), 0.005% to 0.03% (w/v), 0.005% to 0.02% (w/v), 0.005% to 0.01% (w/v), 0.006% to 0.05% (w/v), 0.007% to 0.05% (w/v), 0.008% to 0.05% (w/v), 0.009% to 0.05% (w/v), 0.01% to 0.05% (w/v), 0.02% to 0.05% (w/v), 0.03% to 0.05% (w/v), 0.04% to 0.05% (w/v), or 0.01% (w/v)), at pH 6.2 to 6.8 (e.g., 6.2 to 6.8, 6.2 to 6.7, 6.2 to 6.6, 6.2 to 6.5, 6.2 to 6.4, 6.2 to 6.3, 6.3 to 6.8, 6.4 to 6.8, 6.5 to 6.8, 6.6 to 6.8, 6.7 to 6.8, or about 6.5). In some embodiments, pharmaceutical formulations suitable for use in treating HNSCC in a subject characterized as having radiographic disease progression while on or after having received: (i) pembrolizumab and platinum/5FU, (ii) pembrolizumab monotherapy, or (iii) platinum/5FU and cetuximab, include about 15 mg/mL of the multi-specific binding protein, and also include: about 20 mM citrate, about 6% (w/v) mannitol; and about 0.01% (w/v) polysorbate 80, at about pH 6.5.

In some embodiments, provided herein are methods for treating HNSCC in a subject, where the subject has:
histologically or cytologically documented relapsed or metastatic HNSCC (e.g., tumor locations include oropharynx, oral cavity, hypopharynx, or larynx);
radiographic disease progression while on or after having received: pembrolizumab+platinum/5FU; pembrolizumab monotherapy; or platinum/5FU+cetuximab
received only 1 line of systemic therapy for treatment of relapsed/metastatic disease; and
adequate hematological function defined by WBC count>3×109/L with ANC>1.5×109/L, lymphocyte count>0.5×109/L, platelet count>75×109/L, and hemoglobin>9 g/dL;
adequate hepatic function defined by a total bilirubin level<1.5× the ULN, an AST level<2.5×ULN, and an ALT level<2.5×ULN, or, for subjects with documented metastatic disease to the liver, AST and ALT levels<5×ULN; and/or
adequate renal function defined by an estimated creatinine clearance>50 mL/min according to the Cockcroft-Gault formula or >30 mL/min.

In some embodiments, provided herein are methods of treating relapsed or metastatic colorectal cancer (CRC) in a subject by administering an effective amount of a multi-specific binding protein, or a pharmaceutical formulation thereof. The multi-specific binding protein incorporates: a first antigen-binding site that binds NKG2D; a second antigen-binding site that binds EGFR; and an antibody Fc domain or a portion thereof sufficient to bind CD16, or a third antigen-binding site that binds CD16.

In some embodiments, provided herein are methods of treating relapsed or metastatic CRC in a subject by administering an effective amount of a multi-specific binding protein, or a pharmaceutical formulation thereof. The multi-specific binding protein incorporates: a first antigen binding site that binds NKG2D; a second antigen binding site that binds EGFR with (i) a VH having the CDR1, CDR2, and CDR3 sequences of SEQ ID NOs: 136, 157, and 138, respectively; and a VL having the CDR1, CDR2, and CDR3 sequences of SEQ ID NOs: 140, 141, and 151, respectively (ii) a VH having the CDR1, CDR2, and CDR3 sequences of SEQ ID NOs: 136, 146, and 138, respectively; and a VL having the CDR1, CDR2, and CDR3 sequences of SEQ ID NOs: 140, 141, and 142, respectively; (iii) a VH having the CDR1, CDR2, and CDR3 sequences of SEQ ID NOs: 136, 146, and 138, respectively; and a VL having the CDR1, CDR2, and CDR3 sequences of SEQ ID NOs: 140, 141, and 151, respectively; (iv) a VH having the CDR1, CDR2, and CDR3 sequences of SEQ ID NOs: 136, 137, and 138, respectively; and a VL having the CDR1, CDR2, and CDR3 sequences of SEQ ID NOs: 140, 141, and 151, respectively; or (v) a VH having the CDR1, CDR2, and CDR3 sequences of SEQ ID NOs: 136, 137, and 138, respectively; and a VL having the CDR1, CDR2, and CDR3 sequences of SEQ ID NOs: 140, 141, and 142, respectively; and an antibody Fc domain or a portion thereof sufficient to bind CD16, or a third antigen-binding site that binds CD16.

In some embodiments, provided herein are methods of treating relapsed or metastatic CRC in a subject by administering an effective amount of a multi-specific binding protein, or a pharmaceutical formulation thereof. The multi-specific binding protein incorporates: a first antigen binding site that binds NKG2D with a VH having the CDR1, CDR2, and CDR3 sequences of SEQ ID NOs: 81, 82, and 97 or 112, respectively; and a VL having the CDR1, CDR2, and CDR3 sequences of SEQ ID NOs: 86, 77, and 87, respectively; a second antigen binding site that binds EGFR with (i) a VH having the CDR1, CDR2, and CDR3 sequences of SEQ ID NOs: 136, 157, and 138, respectively; and a VL having the CDR1, CDR2, and CDR3 sequences of SEQ ID NOs: 140, 141, and 151, respectively; (ii) a VH having the CDR1, CDR2, and CDR3 sequences of SEQ ID NOs: 136, 146, and 138, respectively; and a VL having the CDR1, CDR2, and CDR3 sequences of SEQ ID NOs: 140, 141, and 142, respectively; (iii) a VH having the CDR1, CDR2, and CDR3 sequences of SEQ ID NOs: 136, 146, and 138, respectively; and a VL having the CDR1, CDR2, and CDR3 sequences of SEQ ID NOs: 140, 141, and 151, respectively; (iv) a VH having the CDR1, CDR2, and CDR3 sequences of SEQ ID NOs: 136, 137, and 138, respectively; and a VL having the CDR1, CDR2, and CDR3 sequences of SEQ ID NOs: 140, 141, and 151, respectively; or (v) a VH having the CDR1, CDR2, and CDR3 sequences of SEQ ID NOs: 136, 137, and 138, respectively; and a VL having the CDR1, CDR2, and CDR3 sequences of SEQ ID NOs: 140, 141, and 142, respectively; and an antibody Fc domain or a portion thereof sufficient to bind CD16, or a third antigen-binding site that binds CD16.

In some embodiments, provided herein are methods of treating relapsed or metastatic CRC in a subject by administering an effective amount of a multi-specific binding protein, or a pharmaceutical formulation thereof. The multi-specific binding protein incorporates: a first polypeptide with the amino acid sequence of SEQ ID NO:167; (b) a second polypeptide with the amino acid sequence of SEQ ID NO:164; and (c) a third polypeptide with the amino acid sequence of SEQ ID NO:165.

In some embodiments, pharmaceutical formulations suitable for use in treating relapsed or metastatic CRC in a subject have 5 mg/mL to 50 mg/mL of the multi-specific binding protein (e.g., 5 mg/mL to 50 mg/mL, 5 mg/mL to 45 mg/mL, 5 mg/mL to 40 mg/mL, 5 mg/mL to 35 mg/mL, 5 mg/mL to 30 mg/mL, 5 mg/mL to 25 mg/mL, 5 mg/mL to 20 mg/mL, 5 mg/mL to 15 mg/mL, 5 mg/mL to 10 mg/mL, 10 mg/mL to 50 mg/mL, 15 mg/mL to 50 mg/mL, 20 mg/mL to 50 mg/mL, 25 mg/mL to 50 mg/mL, 30 mg/mL to 50 mg/mL, 35 mg/mL to 50 mg/mL, 40 mg/mL to 50 mg/mL, 45 mg/mL to 50 mg/mL, or 15 mg/mL), and also include: 15 mM to 25 mM citrate (e.g., 15 mM to 25 mM, 16 mM to 25 mM, 17 mM to 25 mM, 18 mM to 25 mM, 19 mM to 25 mM, 20 mM to 25 mM, 21 mM to 25 mM, 22 mM to 25 mM, 23 mM to 25 mM, 24 mM to 25 mM, 15 mM to 24 mM, 15 mM to 23 mM, 15 mM to 22 mM, 15 mM to 23 mM, 15 mM to 22 mM, 15 mM to 21 mM, 15 mM to 20 mM, 15 mM to 19 mM, 15 mM to 18 mM, 15 mM to 17 mM, 15 mM to 16 mM, or about 20 mM), 4% to 8% (w/v) mannitol (e.g., 4% to 8% (w/v), 4% to 7% (w/v), 4% to 6% (w/v), 4% to 5% (w/v), 5% to 8% (w/v), 6% to 8% (w/v), 7% to 8% (w/v), or about 6% (w/v)); and 0.005% to 0.05% (w/v) polysorbate 80 (e.g., 0.005% to 0.05% (w/v), 0.005% to 0.04% (w/v), 0.005% to 0.03% (w/v), 0.005% to 0.02% (w/v), 0.005% to 0.01% (w/v), 0.006% to 0.05% (w/v), 0.007% to 0.05% (w/v), 0.008% to 0.05% (w/v), 0.009% to 0.05% (w/v), 0.01% to 0.05% (w/v), 0.02% to 0.05% (w/v), 0.03% to 0.05% (w/v), 0.04% to 0.05% (w/v), or 0.01% (w/v)), at pH 6.2 to 6.8 (e.g., 6.2 to 6.8, 6.2 to 6.7, 6.2 to 6.6, 6.2 to 6.5, 6.2 to 6.4, 6.2 to 6.3, 6.3 to 6.8, 6.4 to 6.8, 6.5 to 6.8, 6.6 to 6.8, 6.7 to 6.8, or about 6.5). In some embodiments, pharmaceutical formulations suitable for use in treating relapsed or metastatic CRC in a subject include about 15 mg/mL of the multi-specific binding protein, and also include: about 20 mM citrate, about 6% (w/v) mannitol; and about 0.01% (w/v) polysorbate 80, at about pH 6.5.

In some embodiments, provided herein are methods of treating colorectal cancer (CRC) in a subject, who has been treated with FOLFOX, CAPOX, FOLFIRI, or FOLFOXIRI, with or without a biological agent, by administering an effective amount of a multi-specific binding protein, or a pharmaceutical formulation thereof. The multi-specific binding protein incorporates: a first antigen-binding site that binds NKG2D; a second antigen-binding site that binds EGFR; and an antibody Fe domain or a portion thereof sufficient to bind CD16, or a third antigen-binding site that binds CD16.

In some embodiments, provided herein are methods of treating CRC in a subject, who has been treated with FOLFOX, CAPOX, FOLFIRI, or FOLFOXIRI, with or without a biological agent, by administering an effective amount of a multi-specific binding protein, or a pharmaceutical formulation thereof. The multi-specific binding protein incorporates: a first antigen binding site that binds NKG2D; a second antigen binding site that binds EGFR with (i) a VH having the CDR1, CDR2, and CDR3 sequences of SEQ ID NOs: 136, 157, and 138, respectively; and a VL having the CDR1, CDR2, and CDR3 sequences of SEQ ID NOs: 140, 141, and 151, respectively; (ii) a VH having the CDR1, CDR2, and CDR3 sequences of SEQ ID NOs: 136, 146, and 138, respectively; and a VL having the CDR1, CDR2, and CDR3 sequences of SEQ ID NOs: 140, 141, and 142, respectively; (iii) a VH having the CDR1, CDR2, and CDR3 sequences of SEQ ID NOs: 136, 146, and 138, respectively; and a VL having the CDR1, CDR2, and CDR3 sequences of SEQ ID NOs: 140, 141, and 151, respectively; (iv) a VH having the CDR1, CDR2, and CDR3 sequences of SEQ ID NOs: 136, 137, and 138, respectively; and a VL having the CDR1, CDR2, and CDR3 sequences of SEQ ID NOs: 140, 141, and 151, respectively; or (v) a VH having the CDR1, CDR2, and CDR3 sequences of SEQ ID NOs: 136, 137, and 138, respectively; and a VL having the CDR1, CDR2, and CDR3 sequences of SEQ ID NOs: 140, 141, and 142, respectively; and an antibody Fc domain or a portion thereof sufficient to bind CD16, or a third antigen-binding site that binds CD16.

In some embodiments, provided herein are methods of treating CRC in a subject, who has been treated with FOLFOX, CAPOX, FOLFIRI, or FOLFOXIRI, with or without a biological agent, by administering an effective amount of a multi-specific binding protein, or a pharmaceutical formulation thereof. The multi-specific binding protein incorporates: a first antigen binding site that binds NKG2D with a VH having the CDR1, CDR2, and CDR3 sequences of SEQ ID NOs: 81, 82, and 97 or 112, respectively; and a VL having the CDR1, CDR2, and CDR3 sequences of SEQ ID NOs: 86, 77, and 87, respectively; a second antigen binding site that binds EGFR with (i) a VH having the CDR1, CDR2, and CDR3 sequences of SEQ ID NOs: 136, 157, and 138, respectively; and a VL having the CDR1, CDR2, and CDR3 sequences of SEQ ID NOs: 140, 141, and 151, respectively; (ii) a VH having the CDR1, CDR2, and CDR3 sequences of SEQ ID NOs: 136, 146, and 138, respectively; and a VL having the CDR1, CDR2, and CDR3 sequences of SEQ ID NOs: 140, 141, and 142, respectively; (iii) a VH having the CDR1, CDR2, and CDR3 sequences of SEQ ID NOs: 136, 146, and 138, respectively; and a VL having the CDR1, CDR2, and CDR3 sequences of SEQ ID NOs: 140, 141, and 151, respectively; (iv) a VH having the CDR1, CDR2, and CDR3 sequences of SEQ ID NOs: 136, 137, and 138, respectively; and a VL having the CDR1, CDR2, and CDR3 sequences of SEQ ID NOs: 140, 141, and 151, respectively; or (v) a VH having the CDR1, CDR2, and CDR3 sequences of SEQ ID NOs: 136, 137, and 138, respectively; and a VL having the CDR1, CDR2, and CDR3 sequences of SEQ ID NOs: 140, 141, and 142, respectively; and an antibody Fc domain or a portion thereof sufficient to bind CD16, or a third antigen-binding site that binds CD16.

In some embodiments, provided herein are methods of treating CRC in a subject, who has been treated with FOLFOX, CAPOX, FOLFIRI, or FOLFOXIRI, with or without a biological agent, by administering an effective amount of a multi-specific binding protein, or a pharmaceutical formulation thereof. The multi-specific binding protein incorporates: a first polypeptide with the amino acid sequence of SEQ ID NO:167; (b) a second polypeptide with the amino acid sequence of SEQ ID NO:164; and (c) a third polypeptide with the amino acid sequence of SEQ ID NO:165.

In some embodiments, pharmaceutical formulations suitable for use in treating CRC in a subject have 5 mg/mL to 50 mg/mL of the multi-specific binding protein (e.g., 5 mg/mL to 50 mg/mL, 5 mg/mL to 45 mg/mL, 5 mg/mL to 40 mg/mL, 5 mg/mL to 35 mg/mL, 5 mg/mL to 30 mg/mL, 5 mg/mL to 25 mg/mL, 5 mg/mL to 20 mg/mL, 5 mg/mL to 15 mg/mL, 5 mg/mL to 10 mg/mL, 10 mg/mL to 50 mg/mL, 15 mg/mL to 50 mg/mL, 20 mg/mL to 50 mg/mL, 25 mg/mL to 50 mg/mL, 30 mg/mL to 50 mg/mL, 35 mg/mL to 50 mg/mL, 40 mg/mL to 50 mg/mL, 45 mg/mL to 50 mg/mL, or 15 mg/mL), and also include: 15 mM to 25 mM citrate (e.g., 15 mM to 25 mM, 16 mM to 25 mM, 17 mM to 25 mM, 18 mM to 25 mM, 19 mM to 25 mM, 20 mM to 25 mM, 21 mM to 25 mM, 22 mM to 25 mM, 23 mM to 25 mM, 24 mM to 25 mM, 15 mM to 24 mM, 15 mM to 23 mM, 15 mM to 22 mM, 15 mM to 23 mM, 15 mM to 22 mM, 15 mM to 21 mM, 15 mM to 20 mM, 15 mM to 19 mM, 15 mM to 18 mM, 15 mM to 17 mM, 15 mM to 16 mM, or about 20 mM), 4% to 8% (w/v) mannitol (e.g., 4% to 8% (w/v), 4% to 7% (w/v), 4% to 6% (w/v), 4% to 5% (w/v), 5% to 8% (w/v), 6% to 8% (w/v), 7% to 8% (w/v), or about 6% (w/v)); and 0.005% to 0.05% (w/v) polysorbate 80 (e.g., 0.005% to 0.05% (w/v), 0.005% to 0.04% (w/v), 0.005% to 0.03% (w/v), 0.005% to 0.02% (w/v), 0.005% to 0.01% (w/v), 0.006% to 0.05% (w/v), 0.007% to 0.05% (w/v), 0.008% to 0.05% (w/v), 0.009% to 0.05% (w/v), 0.01% to 0.05% (w/v), 0.02% to 0.05% (w/v), 0.03% to 0.05% (w/v), 0.04% to 0.05% (w/v), or 0.01% (w/v)), at pH 6.2 to 6.8 (e.g., 6.2 to 6.8, 6.2 to 6.7, 6.2 to 6.6, 6.2 to 6.5, 6.2 to 6.4, 6.2 to 6.3, 6.3 to 6.8, 6.4 to 6.8, 6.5 to 6.8, 6.6 to 6.8, 6.7 to 6.8, or about 6.5), where the subject has been treated with FOLFOX, CAPOX, FOLFIRI, or FOLFOXIRI, with or without a biological agent. In some embodiments, pharmaceutical formulations suitable for use in treating CRC in a subject, who has been treated with FOLFOX, CAPOX, FOLFIRI, or FOLFOXIRI, with or without a biological agent, include about 15 mg/mL of the multi-specific binding protein, and also include: about 20 mM citrate, about 6% (w/v) mannitol; and about 0.01% (w/v) polysorbate 80, at about pH 6.5.

In some embodiments, provided herein are methods of treating colorectal cancer (CRC) in a subject-characterized as not having high mismatch repair/microsatellite instability—by administering an effective amount of a multi-specific binding protein, or a pharmaceutical formulation thereof. The multi-specific binding protein incorporates: a first antigen-binding site that binds NKG2D; a second antigen-binding site that binds EGFR; and an antibody Fc domain or a portion thereof sufficient to bind CD16, or a third antigen-binding site that binds CD16.

In some embodiments, high mismatch repair/microsatellite instability is detected through the comparison of the length of nucleotide repeats in tumor cells and normal cells. The National Cancer Institute standard diagnostic procedure analyses tumor and normal tissues using five microsatellite markers, including two for mononucleotide repeats (BAT26 and BAT25) and three for dinucleotide repeats (D2S123, D5S346, and D17S250). Frame shift mutations in microsatellites can be identified by extraction of DNA from healthy and tumor tissue, amplification of selective microsatellites by PCR, and analysis of fragment size by capillary electrophoresis on an automated sequencer. Samples can be graded as microsatellite instability-high (MSI-H) if two or more of the five microsatellite markers show instability, microsatellite instability-low (MSI-L) if only one of five markers shows instability, and microsatellite stable (MSS) if none of the markers show instability or according the percentage of loci with MSI. Additional methods to determine mismatch repair/microsatellite instability are provided in De'Angelis, Gian Luigi et al. Acta bio-medica: Atenei Parmensis vol. 89(9-S): 97-101, 2018.

In some embodiments, provided herein are methods of treating CRC in a subject-characterized as not having high mismatch repair/microsatellite instability—by administering an effective amount of a multi-specific binding protein, or a pharmaceutical formulation thereof. The multi-specific binding protein incorporates: a first antigen binding site that binds NKG2D; a second antigen binding site that binds EGFR with (i) a VH having the CDR1, CDR2, and CDR3 sequences of SEQ ID NOs: 136, 157, and 138, respectively; and a VL having the CDR1, CDR2, and CDR3 sequences of SEQ ID NOs: 140, 141, and 151, respectively; (ii) a VH having the CDR1, CDR2, and CDR3 sequences of SEQ ID NOs: 136, 146, and 138, respectively; and a VL having the CDR1, CDR2, and CDR3 sequences of SEQ ID NOs: 140, 141, and 142, respectively; (iii) a VH having the CDR1, CDR2, and CDR3 sequences of SEQ ID NOs: 136, 146, and 138, respectively; and a VL having the CDR1, CDR2, and CDR3 sequences of SEQ ID NOs: 140, 141, and 151, respectively; (iv) a VH having the CDR1, CDR2, and CDR3 sequences of SEQ ID NOs: 136, 137, and 138, respectively; and a VL having the CDR1, CDR2, and CDR3 sequences of SEQ ID NOs: 140, 141, and 151, respectively; or (v) a VH having the CDR1, CDR2, and CDR3 sequences of SEQ ID NOs: 136, 137, and 138, respectively; and a VL having the CDR1, CDR2, and CDR3 sequences of SEQ ID NOs: 140, 141, and 142, respectively; and an antibody Fc domain or a portion thereof sufficient to bind CD16, or a third antigen-binding site that binds CD16.

In some embodiments, provided herein are methods of treating CRC in a subject-characterized as not having high mismatch repair/microsatellite instability—by administering an effective amount of a multi-specific binding protein, or a pharmaceutical formulation thereof. The multi-specific binding protein incorporates: a first antigen binding site that binds NKG2D with a VH having the CDR1, CDR2, and CDR3 sequences of SEQ ID NOs: 81, 82, and 97 or 112, respectively; and a VL having the CDR1, CDR2, and CDR3 sequences of SEQ ID NOs: 86, 77, and 87, respectively; a second antigen binding site that binds EGFR with (i) a VH having the CDR1, CDR2, and CDR3 sequences of SEQ ID NOs: 136, 157, and 138, respectively; and a VL having the CDR1, CDR2, and CDR3 sequences of SEQ ID NOs: 140, 141, and 151, respectively; (ii) a VH having the CDR1, CDR2, and CDR3 sequences of SEQ ID NOs: 136, 146, and 138, respectively; and a VL having the CDR1, CDR2, and CDR3 sequences of SEQ ID NOs: 140, 141, and 142, respectively; (iii) a VH having the CDR1, CDR2, and CDR3 sequences of SEQ ID NOs: 136, 146, and 138, respectively; and a VL having the CDR1, CDR2, and CDR3 sequences of SEQ ID NOs: 140, 141, and 151, respectively; (iv) a VH having the CDR1, CDR2, and CDR3 sequences of SEQ ID NOs: 136, 137, and 138, respectively; and a VL having the CDR1, CDR2, and CDR3 sequences of SEQ ID NOs: 140, 141, and 151, respectively; or (v) a VH having the CDR1, CDR2, and CDR3 sequences of SEQ ID NOs: 136, 137, and 138, respectively; and a VL having the CDR1, CDR2, and CDR3 sequences of SEQ ID NOs: 140, 141, and 142, respectively; and an antibody Fc domain or a portion thereof sufficient to bind CD16, or a third antigen-binding site that binds CD16.

In some embodiments, provided herein are methods of treating CRC in a subject-characterized as not having high mismatch repair/microsatellite instability—by administering an effective amount of a multi-specific binding protein, or a pharmaceutical formulation thereof. The multi-specific binding protein incorporates: a first polypeptide with the amino acid sequence of SEQ ID NO:167; (b) a second polypeptide with the amino acid sequence of SEQ ID NO:164; and (c) a third polypeptide with the amino acid sequence of SEQ ID NO:165.

In some embodiments, pharmaceutical formulations suitable for use in treating CRC in a subject-characterized as not having high mismatch repair/microsatellite instability—have 5 mg/mL to 50 mg/mL of the multi-specific binding protein (e.g., 5 mg/mL to 50 mg/mL, 5 mg/mL to 45 mg/mL, 5 mg/mL to 40 mg/mL, 5 mg/mL to 35 mg/mL, 5 mg/mL to 30 mg/mL, 5 mg/mL to 25 mg/mL, 5 mg/mL to 20 mg/mL, 5 mg/mL to 15 mg/mL, 5 mg/mL to 10 mg/mL, 10 mg/mL to 50 mg/mL, 15 mg/mL to 50 mg/mL, 20 mg/mL to 50 mg/mL, 25 mg/mL to 50 mg/mL, 30 mg/mL to 50 mg/mL, 35 mg/mL to 50 mg/mL, 40 mg/mL to 50 mg/mL, 45 mg/mL to 50 mg/mL, or 15 mg/mL), and also include: 15 mM to 25 mM citrate (e.g., 15 mM to 25 mM, 16 mM to 25 mM, 17 mM to 25 mM, 18 mM to 25 mM, 19 mM to 25 mM, 20 mM to 25 mM, 21 mM to 25 mM, 22 mM to 25 mM, 23 mM to 25 mM, 24 mM to 25 mM, 15 mM to 24 mM, 15 mM to 23 mM, 15 mM to 22 mM, 15 mM to 23 mM, 15 mM to 22 mM, 15 mM to 21 mM, 15 mM to 20 mM, 15 mM to 19 mM, 15 mM to 18 mM, 15 mM to 17 mM, 15 mM to 16 mM, or about 20 mM), 4% to 8% (w/v) mannitol (e.g., 4% to 8% (w/v), 4% to 7% (w/v), 4% to 6% (w/v), 4% to 5% (w/v), 5% to 8% (w/v), 6% to 8% (w/v), 7% to 8% (w/v), or about 6% (w/v)); and 0.005% to 0.05% (w/v) polysorbate 80 (e.g., 0.005% to 0.05% (w/v), 0.005% to 0.04% (w/v), 0.005% to 0.03% (w/v), 0.005% to 0.02% (w/v), 0.005% to 0.01% (w/v), 0.006% to 0.05% (w/v), 0.007% to 0.05% (w/v), 0.008% to 0.05% (w/v), 0.009% to 0.05% (w/v), 0.01% to 0.05% (w/v), 0.02% to 0.05% (w/v), 0.03% to 0.05% (w/v), 0.04% to 0.05% (w/v), or 0.01% (w/v)), at pH 6.2 to 6.8 (e.g., 6.2 to 6.8, 6.2 to 6.7, 6.2 to 6.6, 6.2 to 6.5, 6.2 to 6.4, 6.2 to 6.3, 6.3 to 6.8, 6.4 to 6.8, 6.5 to 6.8, 6.6 to 6.8, 6.7 to 6.8, or about 6.5). In some embodiments, pharmaceutical formulations suitable for use in treating CRC in a subject-characterized as not having high mismatch repair/microsatellite instability-include about 15 mg/mL of the multi-specific binding protein, and also include: about 20 mM citrate, about 6% (w/v) mannitol; and about 0.01% (w/v) polysorbate 80, at about pH 6.5.

In some embodiments, provided herein are methods of treating colorectal cancer (CRC) in a subject-characterized as not previously treated with an anti-PD-1 or an anti-PD-L1 therapy—by administering an effective amount of a multi-specific binding protein, or a pharmaceutical formulation thereof. The multi-specific binding protein incorporates: a first antigen-binding site that binds NKG2D; a second antigen-binding site that binds EGFR; and an antibody Fc domain or a portion thereof sufficient to bind CD16, or a third antigen-binding site that binds CD16.

In some embodiments, provided herein are methods of treating CRC in a subject-characterized as not previously treated with an anti-PD-1 or an anti-PD-L1 therapy—by administering an effective amount of a multi-specific binding protein, or a pharmaceutical formulation thereof. The multi-specific binding protein incorporates: a first antigen binding site that binds NKG2D; a second antigen binding site that binds EGFR with (i) a VH having the CDR1, CDR2, and CDR3 sequences of SEQ ID NOs: 136, 157, and 138, respectively; and a VL having the CDR1, CDR2, and CDR3 sequences of SEQ ID NOs: 140, 141, and 151, respectively; (ii) a VH having the CDR1, CDR2, and CDR3 sequences of SEQ ID NOs: 136, 146, and 138, respectively; and a VL having the CDR1, CDR2, and CDR3 sequences of SEQ ID NOs: 140, 141, and 142, respectively; (iii) a VH having the CDR1, CDR2, and CDR3 sequences of SEQ ID NOs: 136, 146, and 138, respectively; and a VL having the CDR1, CDR2, and CDR3 sequences of SEQ ID NOs: 140, 141, and 151, respectively; (iv) a VH having the CDR1, CDR2, and CDR3 sequences of SEQ ID NOs: 136, 137, and 138, respectively; and a VL having the CDR1, CDR2, and CDR3 sequences of SEQ ID NOs: 140, 141, and 151, respectively; or (v) a VH having the CDR1, CDR2, and CDR3 sequences of SEQ ID NOs: 136, 137, and 138, respectively; and a VL having the CDR1, CDR2, and CDR3 sequences of SEQ ID NOs: 140, 141, and 142, respectively; and an antibody Fc domain or a portion thereof sufficient to bind CD16, or a third antigen-binding site that binds CD16.

In some embodiments, provided herein are methods of treating CRC in a subject-characterized as not previously treated with an anti-PD-1 or an anti-PD-L1 therapy—by administering an effective amount of a multi-specific binding protein, or a pharmaceutical formulation thereof. The multi-specific binding protein incorporates: a first antigen binding site that binds NKG2D with a VH having the CDR1, CDR2, and CDR3 sequences of SEQ ID NOs: 81, 82, and 97 or 112, respectively; and a VL having the CDR1, CDR2, and CDR3 sequences of SEQ ID NOs: 86, 77, and 87, respectively; a second antigen binding site that binds EGFR with (i) a VH having the CDR1, CDR2, and CDR3 sequences of SEQ ID NOs: 136, 157, and 138, respectively; and a VL having the CDR1, CDR2, and CDR3 sequences of SEQ ID NOs: 140, 141, and 151, respectively; (ii) a VH having the CDR1, CDR2, and CDR3 sequences of SEQ ID NOs: 136, 146, and 138, respectively; and a VL having the CDR1, CDR2, and CDR3 sequences of SEQ ID NOs: 140, 141, and 142, respectively; (iii) a VH having the CDR1, CDR2, and CDR3 sequences of SEQ ID NOs: 136, 146, and 138, respectively; and a VL having the CDR1, CDR2, and CDR3 sequences of SEQ ID NOs: 140, 141, and 151, respectively; (iv) a VH having the CDR1, CDR2, and CDR3 sequences of SEQ ID NOs: 136, 137, and 138, respectively; and a VL having the CDR1, CDR2, and CDR3 sequences of SEQ ID NOs: 140, 141, and 151, respectively; or (v) a VH having the CDR1, CDR2, and CDR3 sequences of SEQ ID NOs: 136, 137, and 138, respectively; and a VL having the CDR1, CDR2, and CDR3 sequences of SEQ ID NOs: 140, 141, and 142, respectively; and an antibody Fc domain or a portion thereof sufficient to bind CD16, or a third antigen-binding site that binds CD16.

In some embodiments, provided herein are methods of treating CRC in a subject-characterized as not previously treated with an anti-PD-1 or an anti-PD-L1 therapy—by administering an effective amount of a multi-specific binding protein, or a pharmaceutical formulation thereof. The multi-specific binding protein incorporates: a first polypeptide with the amino acid sequence of SEQ ID NO:167; (b) a second polypeptide with the amino acid sequence of SEQ ID NO:164; and (c) a third polypeptide with the amino acid sequence of SEQ ID NO:165.

In some embodiments, pharmaceutical formulations suitable for use in treating CRC in a subject-characterized as not previously treated with an anti-PD-1 or an anti-PD-L1 therapy—have 5 mg/mL to 50 mg/mL of the multi-specific binding protein (e.g., 5 mg/mL to 50 mg/mL, 5 mg/mL to 45 mg/mL, 5 mg/mL to 40 mg/mL, 5 mg/mL to 35 mg/mL, 5 mg/mL to 30 mg/mL, 5 mg/mL to 25 mg/mL, 5 mg/mL to 20 mg/mL, 5 mg/mL to 15 mg/mL, 5 mg/mL to 10 mg/mL, 10 mg/mL to 50 mg/mL, 15 mg/mL to 50 mg/mL, 20 mg/mL to 50 mg/mL, 25 mg/mL to 50 mg/mL, 30 mg/mL to 50 mg/mL, 35 mg/mL to 50 mg/mL, 40 mg/mL to 50 mg/mL, 45 mg/mL to 50 mg/mL, or 15 mg/mL), and also include: 15 mM to 25 mM citrate (e.g., 15 mM to 25 mM, 16 mM to 25 mM, 17 mM to 25 mM, 18 mM to 25 mM, 19 mM to 25 mM, 20 mM to 25 mM, 21 mM to 25 mM, 22 mM to 25 mM, 23 mM to 25 mM, 24 mM to 25 mM, 15 mM to 24 mM, 15 mM to 23 mM, 15 mM to 22 mM, 15 mM to 23 mM, 15 mM to 22 mM, 15 mM to 21 mM, 15 mM to 20 mM, 15 mM to 19 mM, 15 mM to 18 mM, 15 mM to 17 mM, 15 mM to 16 mM, or about 20 mM), 4% to 8% (w/v) mannitol (e.g., 4% to 8% (w/v), 4% to 7% (w/v), 4% to 6% (w/v), 4% to 5% (w/v), 5% to 8% (w/v), 6% to 8% (w/v), 7% to 8% (w/v), or about 6% (w/v)); and 0.005% to 0.05% (w/v) polysorbate 80 (e.g., 0.005% to 0.05% (w/v), 0.005% to 0.04% (w/v), 0.005% to 0.03% (w/v), 0.005% to 0.02% (w/v), 0.005% to 0.01% (w/v), 0.006% to 0.05% (w/v), 0.007% to 0.05% (w/v), 0.008% to 0.05% (w/v), 0.009% to 0.05% (w/v), 0.01% to 0.05% (w/v), 0.02% to 0.05% (w/v), 0.03% to 0.05% (w/v), 0.04% to 0.05% (w/v), or 0.01% (w/v)), at pH 6.2 to 6.8 (e.g., 6.2 to 6.8, 6.2 to 6.7, 6.2 to 6.6, 6.2 to 6.5, 6.2 to 6.4, 6.2 to 6.3, 6.3 to 6.8, 6.4 to 6.8, 6.5 to 6.8, 6.6 to 6.8, 6.7 to 6.8, or about 6.5). In some embodiments, pharmaceutical formulations suitable for use in treating CRC in a subject-characterized as not previously treated with an anti-PD-1 or an anti-PD-L1 therapy-include about 15 mg/mL of the multi-specific binding protein, and also include: about 20 mM citrate, about 6% (w/v) mannitol; and about 0.01% (w/v) polysorbate 80, at about pH 6.5.

In some embodiments, provided herein are methods of treating colorectal cancer (CRC) in a subject-characterized as having radiographic disease progression while or after receiving treatment for advanced (recurrent/unresectable/metastatic) cancer—by administering an effective amount of a multi-specific binding protein, or a pharmaceutical formulation thereof. The multi-specific binding protein incorporates: a first antigen-binding site that binds NKG2D; a second antigen-binding site that binds EGFR; and an antibody Fc domain or a portion thereof sufficient to bind CD16, or a third antigen-binding site that binds CD16.

In some embodiments, provided herein are methods of treating CRC in a subject-characterized as having radiographic disease progression while or after receiving treatment for advanced (recurrent/unresectable/metastatic) cancer—by administering an effective amount of a multi-specific binding protein, or a pharmaceutical formulation thereof. The multi-specific binding protein incorporates: a first antigen binding site that binds NKG2D; a second antigen binding site that binds EGFR with (i) a VH having the CDR1, CDR2, and CDR3 sequences of SEQ ID NOs: 136, 157, and 138, respectively; and a VL having the CDR1, CDR2, and CDR3 sequences of SEQ ID NOs: 140, 141, and 151, respectively; (ii) a VH having the CDR1, CDR2, and CDR3 sequences of SEQ ID NOs: 136, 146, and 138, respectively; and a VL having the CDR1, CDR2, and CDR3 sequences of SEQ ID NOs: 140, 141, and 142, respectively; (iii) a VH having the CDR1, CDR2, and CDR3 sequences of SEQ ID NOs: 136, 146, and 138, respectively; and a VL having the CDR1, CDR2, and CDR3 sequences of SEQ ID NOs: 140, 141, and 151, respectively; (iv) a VH having the CDR1, CDR2, and CDR3 sequences of SEQ ID NOs: 136, 137, and 138, respectively; and a VL having the CDR1, CDR2, and CDR3 sequences of SEQ ID NOs: 140, 141, and 151, respectively; or (v) a VH having the CDR1, CDR2, and CDR3 sequences of SEQ ID NOs: 136, 137, and 138, respectively; and a VL having the CDR1, CDR2, and CDR3 sequences of SEQ ID NOs: 140, 141, and 142, respectively; and an antibody Fc domain or a portion thereof sufficient to bind CD16, or a third antigen-binding site that binds CD16.

In some embodiments, provided herein are methods of treating CRC in a subject-characterized as having radiographic disease progression while or after receiving treatment for advanced (recurrent/unresectable/metastatic) cancer—by administering an effective amount of a multi-specific binding protein, or a pharmaceutical formulation thereof. The multi-specific binding protein incorporates: a first antigen binding site that binds NKG2D with a VH having the CDR1, CDR2, and CDR3 sequences of SEQ ID NOs: 81, 82, and 97 or 112, respectively; and a VL having the CDR1, CDR2, and CDR3 sequences of SEQ ID NOs: 86, 77, and 87, respectively; a second antigen binding site that binds EGFR with (i) a VH having the CDR1, CDR2, and CDR3 sequences of SEQ ID NOs: 136, 157, and 138, respectively; and a VL having the CDR1, CDR2, and CDR3 sequences of SEQ ID NOs: 140, 141, and 151, respectively; (ii) a VH having the CDR1, CDR2, and CDR3 sequences of SEQ ID NOs: 136, 146, and 138, respectively; and a VL having the CDR1, CDR2, and CDR3 sequences of SEQ ID NOs: 140, 141, and 142, respectively; (iii) a VH having the CDR1, CDR2, and CDR3 sequences of SEQ ID NOs: 136, 146, and 138, respectively; and a VL having the CDR1, CDR2, and CDR3 sequences of SEQ ID NOs: 140, 141, and 151, respectively; (iv) a VH having the CDR1, CDR2, and CDR3 sequences of SEQ ID NOs: 136, 137, and 138, respectively; and a VL having the CDR1, CDR2, and CDR3 sequences of SEQ ID NOs: 140, 141, and 151, respectively; or (v) a VH having the CDR1, CDR2, and CDR3 sequences of SEQ ID NOs: 136, 137, and 138, respectively; and a VL having the CDR1, CDR2, and CDR3 sequences of SEQ ID NOs: 140, 141, and 142, respectively; and an antibody Fc domain or a portion thereof sufficient to bind CD16, or a third antigen-binding site that binds CD16.

In some embodiments, provided herein are methods of treating CRC in a subject-characterized as having radiographic disease progression while or after receiving treatment for advanced (recurrent/unresectable/metastatic) cancer—by administering an effective amount of a multi-specific binding protein, or a pharmaceutical formulation thereof. The multi-specific binding protein incorporates: a first polypeptide with the amino acid sequence of SEQ ID NO:167; (b) a second polypeptide with the amino acid sequence of SEQ ID NO:164; and (c) a third polypeptide with the amino acid sequence of SEQ ID NO:165.

In some embodiments, pharmaceutical formulations suitable for use in treating CRC in a subject-characterized as having radiographic disease progression while or after receiving treatment for advanced (recurrent/unresectable/metastatic) cancer—have 5 mg/mL to 50 mg/mL of the multi-specific binding protein (e.g., 5 mg/mL to 50 mg/mL, 5 mg/mL to 45 mg/mL, 5 mg/mL to 40 mg/mL, 5 mg/mL to 35 mg/mL, 5 mg/mL to 30 mg/mL, 5 mg/mL to 25 mg/mL, 5 mg/mL to 20 mg/mL, 5 mg/mL to 15 mg/mL, 5 mg/mL to 10 mg/mL, 10 mg/mL to 50 mg/mL, 15 mg/mL to 50 mg/mL, 20 mg/mL to 50 mg/mL, 25 mg/mL to 50 mg/mL, 30 mg/mL to 50 mg/mL, 35 mg/mL to 50 mg/mL, 40 mg/mL to 50 mg/mL, 45 mg/mL to 50 mg/mL, or 15 mg/mL), and also include: 15 mM to 25 mM citrate (e.g., 15 mM to 25 mM, 16 mM to 25 mM, 17 mM to 25 mM, 18 mM to 25 mM, 19 mM to 25 mM, 20 mM to 25 mM, 21 mM to 25 mM, 22 mM to 25 mM, 23 mM to 25 mM, 24 mM to 25 mM, 15 mM to 24 mM, 15 mM to 23 mM, 15 mM to 22 mM, 15 mM to 23 mM, 15 mM to 22 mM, 15 mM to 21 mM, 15 mM to 20 mM, 15 mM to 19 mM, 15 mM to 18 mM, 15 mM to 17 mM, 15 mM to 16 mM, or about 20 mM), 4% to 8% (w/v) mannitol (e.g., 4% to 8% (w/v), 4% to 7% (w/v), 4% to 6% (w/v), 4% to 5% (w/v), 5% to 8% (w/v), 6% to 8% (w/v), 7% to 8% (w/v), or about 6% (w/v)); and 0.005% to 0.05% (w/v) polysorbate 80 (e.g., 0.005% to 0.05% (w/v), 0.005% to 0.04% (w/v), 0.005% to 0.03% (w/v), 0.005% to 0.02% (w/v), 0.005% to 0.01% (w/v), 0.006% to 0.05% (w/v), 0.007% to 0.05% (w/v), 0.008% to 0.05% (w/v), 0.009% to 0.05% (w/v), 0.01% to 0.05% (w/v), 0.02% to 0.05% (w/v), 0.03% to 0.05% (w/v), 0.04% to 0.05% (w/v), or 0.01% (w/v)), at pH 6.2 to 6.8 (e.g., 6.2 to 6.8, 6.2 to 6.7, 6.2 to 6.6, 6.2 to 6.5, 6.2 to 6.4, 6.2 to 6.3, 6.3 to 6.8, 6.4 to 6.8, 6.5 to 6.8, 6.6 to 6.8, 6.7 to 6.8, or about 6.5). In some embodiments, pharmaceutical formulations suitable for use in treating CRC in a subject-characterized as having radiographic disease progression while or after receiving treatment for advanced (recurrent/unresectable/metastatic) cancer-include about 15 mg/mL of the multi-specific binding protein, and also include: about 20 mM citrate, about 6% (w/v) mannitol; and about 0.01% (w/v) polysorbate 80, at about pH 6.5.

In some embodiments, provided herein are methods for treating CRC in a subject, where the subject:
has histologically or cytologically documented relapsed or metastatic CRC;
has received 1 line of either FOLFOX, CAPOX, FOLFIRI, or FOLFOXIRI with or without a biological agent;
has received cetuximab if the subject is KRAS wild-type;
should not be mismatch repair (MMR)/microsatellite instability (MSI) high;
should not have received an anti-PD-1 or anti-PD-L1 therapy;
has radiographic disease progression while or after receiving treatment for their advanced (recurrent/unresectable/metastatic) disease;
has adequate hematological function defined by WBC count>3×109/L, with ANC>1.5×109/L, lymphocyte count>0.5×109/L, platelet count>75×109/L, and hemoglobin>9 g/dL;
has adequate hepatic function defined by a total bilirubin level<1.5× the ULN, an AST level<2.5×ULN, and an ALT level<2.5×ULN, or, for patients with documented metastatic disease to the liver, AST and ALT levels<5×ULN; and/or
has adequate renal function defined by an estimated creatinine clearance>50 mL/min according to the Cockcroft-Gault formula or >30 mL/min.

In some embodiments, provided herein are methods of treating non-small-cell lung cancer (NSCLC) in a subject-characterized as having recurrent or progressive disease during or after platinum doublet-based chemotherapy, or having recurrent or progressive disease within 6 months after completing platinum-based chemotherapy for local disease—by administering an effective amount of a multi-specific binding protein, or a pharmaceutical formulation thereof. The multi-specific binding protein incorporates: a first antigen-binding site that binds NKG2D; a second antigen-binding site that binds EGFR; and an antibody Fc domain or a portion thereof sufficient to bind CD16, or a third antigen-binding site that binds CD16.

In some embodiments, provided herein are methods of treating NSCLC in a subject-characterized as having recurrent or progressive disease during or after platinum doublet-based chemotherapy, or having recurrent or progressive disease within 6 months after completing platinum-based chemotherapy for local disease—by administering an effective amount of a multi-specific binding protein, or a pharmaceutical formulation thereof. The multi-specific binding protein incorporates: a first antigen binding site that binds NKG2D; a second antigen binding site that binds EGFR with (i) a VH having the CDR1, CDR2, and CDR3 sequences of SEQ ID NOs: 136, 157, and 138, respectively; and a VL having the CDR1, CDR2, and CDR3 sequences of SEQ ID NOs: 140, 141, and 151, respectively; (ii) a VH having the CDR1, CDR2, and CDR3 sequences of SEQ ID NOs: 136, 146, and 138, respectively; and a VL having the CDR1, CDR2, and CDR3 sequences of SEQ ID NOs: 140, 141, and 142, respectively; (iii) a VH having the CDR1, CDR2, and CDR3 sequences of SEQ ID NOs: 136, 146, and 138, respectively; and a VL having the CDR1, CDR2, and CDR3 sequences of SEQ ID NOs: 140, 141, and 151, respectively; (iv) a VH having the CDR1, CDR2, and CDR3 sequences of SEQ ID NOs: 136, 137, and 138, respectively; and a VL having the CDR1, CDR2, and CDR3 sequences of SEQ ID NOs: 140, 141, and 151, respectively; or (v) a VH having the CDR1, CDR2, and CDR3 sequences of SEQ ID NOs: 136, 137, and 138, respectively; and a VL having the CDR1, CDR2, and CDR3 sequences of SEQ ID NOs: 140, 141, and 142, respectively; and an antibody Fc domain or a portion thereof sufficient to bind CD16, or a third antigen-binding site that binds CD16.

In some embodiments, provided herein are methods of treating NSCLC in a subject-characterized as having recurrent or progressive disease during or after platinum doublet-based chemotherapy, or having recurrent or progressive disease within 6 months after completing platinum-based chemotherapy for local disease—by administering an effective amount of a multi-specific binding protein, or a pharmaceutical formulation thereof. The multi-specific binding protein incorporates: a first antigen binding site that binds NKG2D with a VH having the CDR1, CDR2, and CDR3 sequences of SEQ ID NOs: 81, 82, and 97 or 112, respectively; and a VL having the CDR1, CDR2, and CDR3 sequences of SEQ ID NOs: 86, 77, and 87, respectively; a second antigen binding site that binds EGFR with (i) a VH having the CDR1, CDR2, and CDR3 sequences of SEQ ID NOs: 136, 157, and 138, respectively; and a VL having the CDR1, CDR2, and CDR3 sequences of SEQ ID NOs: 140, 141, and 151, respectively; (ii) a VH having the CDR1, CDR2, and CDR3 sequences of SEQ ID NOs: 136, 146, and 138, respectively; and a VL having the CDR1, CDR2, and CDR3 sequences of SEQ ID NOs: 140, 141, and 142, respectively; (iii) a VH having the CDR1, CDR2, and CDR3 sequences of SEQ ID NOs: 136, 146, and 138, respectively; and a VL having the CDR1, CDR2, and CDR3 sequences of SEQ ID NOs: 140, 141, and 151, respectively; (iv) a VH having the CDR1, CDR2, and CDR3 sequences of SEQ ID NOs: 136, 137, and 138, respectively; and a VL having the CDR1, CDR2, and CDR3 sequences of SEQ ID NOs: 140, 141, and 151, respectively; or (v) a VH having the CDR1, CDR2, and CDR3 sequences of SEQ ID NOs: 136, 137, and 138, respectively; and a VL having the CDR1, CDR2, and CDR3 sequences of SEQ ID NOs: 140, 141, and 142, respectively; and an antibody Fc domain or a portion thereof sufficient to bind CD16, or a third antigen-binding site that binds CD16.

In some embodiments, provided herein are methods of treating NSCLC in a subject-characterized as having recurrent or progressive disease during or after platinum doublet-based chemotherapy, or having recurrent or progressive disease within 6 months after completing platinum-based chemotherapy for local disease—by administering an effective amount of a multi-specific binding protein, or a pharmaceutical formulation thereof. The multi-specific binding protein incorporates: a first polypeptide with the amino acid sequence of SEQ ID NO:167; (b) a second polypeptide with the amino acid sequence of SEQ ID NO:164; and (c) a third polypeptide with the amino acid sequence of SEQ ID NO:165.

In some embodiments, pharmaceutical formulations suitable for use in treating NSCLC in a subject-characterized as having recurrent or progressive disease during or after platinum doublet-based chemotherapy, or having recurrent or progressive disease within 6 months after completing platinum-based chemotherapy for local disease—have 5 mg/mL to 50 mg/mL of the multi-specific binding protein (e.g., 5 mg/mL to 50 mg/mL, 5 mg/mL to 45 mg/mL, 5 mg/mL to 40 mg/mL, 5 mg/mL to 35 mg/mL, 5 mg/mL to 30 mg/mL, 5 mg/mL to 25 mg/mL, 5 mg/mL to 20 mg/mL, 5 mg/mL to 15 mg/mL, 5 mg/mL to 10 mg/mL, 10 mg/mL to 50 mg/mL, 15 mg/mL to 50 mg/mL, 20 mg/mL to 50 mg/mL, 25 mg/mL to 50 mg/mL, 30 mg/mL to 50 mg/mL, 35 mg/mL to 50 mg/mL, 40 mg/mL to 50 mg/mL, 45 mg/mL to 50 mg/mL, or 15 mg/mL), and also include: 15 mM to 25 mM citrate (e.g., 15 mM to 25 mM, 16 mM to 25 mM, 17 mM to 25 mM, 18 mM to 25 mM, 19 mM to 25 mM, 20 mM to 25 mM, 21 mM to 25 mM, 22 mM to 25 mM, 23 mM to 25 mM, 24 mM to 25 mM, 15 mM to 24 mM, 15 mM to 23 mM, 15 mM to 22 mM, 15 mM to 23 mM, 15 mM to 22 mM, 15 mM to 21 mM, 15 mM to 20 mM, 15 mM to 19 mM, 15 mM to 18 mM, 15 mM to 17 mM, 15 mM to 16 mM, or about 20 mM), 4% to 8% (w/v) mannitol (e.g., 4% to 8% (w/v), 4% to 7% (w/v), 4% to 6% (w/v), 4% to 5% (w/v), 5% to 8% (w/v), 6% to 8% (w/v), 7% to 8% (w/v), or about 6% (w/v)); and 0.005% to 0.05% (w/v) polysorbate 80 (e.g., 0.005% to 0.05% (w/v), 0.005% to 0.04% (w/v), 0.005% to 0.03% (w/v), 0.005% to 0.02% (w/v), 0.005% to 0.01% (w/v), 0.006% to 0.05% (w/v), 0.007% to 0.05% (w/v), 0.008% to 0.05% (w/v), 0.009% to 0.05% (w/v), 0.01% to 0.05% (w/v), 0.02% to 0.05% (w/v), 0.03% to 0.05% (w/v), 0.04% to 0.05% (w/v), or 0.01% (w/v)), at pH 6.2 to 6.8 (e.g., 6.2 to 6.8, 6.2 to 6.7, 6.2 to 6.6, 6.2 to 6.5, 6.2 to 6.4, 6.2 to 6.3, 6.3 to 6.8, 6.4 to 6.8, 6.5 to 6.8, 6.6 to 6.8, 6.7 to 6.8, or about 6.5). In some embodiments, pharmaceutical formulations suitable for use in treating NSCLC in a subject-characterized as having recurrent or progressive disease during or after platinum doublet-based chemotherapy, or having recurrent or progressive disease within 6 months after completing platinum-based chemotherapy for local disease-include about 15 mg/mL of the multi-specific binding protein, and also include: about 20 mM citrate, about 6% (w/v) mannitol; and about 0.01% (w/v) polysorbate 80, at about pH 6.5.

In some embodiments, provided herein are methods of treating non-small-cell lung cancer (NSCLC) in a subject, who has previously received an anti-PD-1 or anti-PD-L1 therapy, by administering an effective amount of a multi-specific binding protein, or a pharmaceutical formulation thereof. The multi-specific binding protein incorporates: a first antigen-binding site that binds NKG2D; a second antigen-binding site that binds EGFR; and an antibody Fc domain or a portion thereof sufficient to bind CD16, or a third antigen-binding site that binds CD16.

In some embodiments, provided herein are methods of treating NSCLC in a subject, who has previously received an anti-PD-1 or anti-PD-L1 therapy, by administering an effective amount of a multi-specific binding protein, or a pharmaceutical formulation thereof. The multi-specific binding protein incorporates: a first antigen binding site that binds NKG2D; a second antigen binding site that binds EGFR with (i) a VH having the CDR1, CDR2, and CDR3 sequences of SEQ ID NOs: 136, 157, and 138, respectively; and a VL having the CDR1, CDR2, and CDR3 sequences of SEQ ID NOs: 140, 141, and 151, respectively; (ii) a VH having the CDR1, CDR2, and CDR3 sequences of SEQ ID NOs: 136, 146, and 138, respectively; and a VL having the CDR1, CDR2, and CDR3 sequences of SEQ ID NOs: 140, 141, and 142, respectively; (iii) a VH having the CDR1, CDR2, and CDR3 sequences of SEQ ID NOs: 136, 146, and 138, respectively; and a VL having the CDR1, CDR2, and CDR3 sequences of SEQ ID NOs: 140, 141, and 151, respectively; (iv) a VH having the CDR1, CDR2, and CDR3 sequences of SEQ ID NOs: 136, 137, and 138, respectively; and a VL having the CDR1, CDR2, and CDR3 sequences of SEQ ID NOs: 140, 141, and 151, respectively; or (v) a VH having the CDR1, CDR2, and CDR3 sequences of SEQ ID NOs: 136, 137, and 138, respectively; and a VL having the CDR1, CDR2, and CDR3 sequences of SEQ ID NOs: 140, 141, and 142, respectively; and an antibody Fc domain or a portion thereof sufficient to bind CD16, or a third antigen-binding site that binds CD16.

In some embodiments, provided herein are methods of treating NSCLC in a subject, who has previously received an anti-PD-1 or anti-PD-L1 therapy, by administering an effective amount of a multi-specific binding protein, or a pharmaceutical formulation thereof. The multi-specific binding protein incorporates: a first antigen binding site that binds NKG2D with a VH having the CDR1, CDR2, and CDR3 sequences of SEQ ID NOs: 81, 82, and 97 or 112, respectively; and a VL having the CDR1, CDR2, and CDR3 sequences of SEQ ID NOs: 86, 77, and 87, respectively; a second antigen binding site that binds EGFR with (i) a VH having the CDR1, CDR2, and CDR3 sequences of SEQ ID NOs: 136, 157, and 138, respectively; and a VL having the CDR1, CDR2, and CDR3 sequences of SEQ ID NOs: 140, 141, and 151, respectively; (ii) a VH having the CDR1, CDR2, and CDR3 sequences of SEQ ID NOs: 136, 146, and 138, respectively; and a VL having the CDR1, CDR2, and CDR3 sequences of SEQ ID NOs: 140, 141, and 142, respectively; (iii) a VH having the CDR1, CDR2, and CDR3 sequences of SEQ ID NOs: 136, 146, and 138, respectively; and a VL having the CDR1, CDR2, and CDR3 sequences of SEQ ID NOs: 140, 141, and 151, respectively; (iv) a VH having the CDR1, CDR2, and CDR3 sequences of SEQ ID NOs: 136, 137, and 138, respectively; and a VL having the CDR1, CDR2, and CDR3 sequences of SEQ ID NOs: 140, 141, and 151, respectively; or (v) a VH having the CDR1, CDR2, and CDR3 sequences of SEQ ID NOs: 136, 137, and 138, respectively; and a VL having the CDR1, CDR2, and CDR3 sequences of SEQ ID NOs: 140, 141, and 142, respectively; and an antibody Fc domain or a portion thereof sufficient to bind CD16, or a third antigen-binding site that binds CD16.

In some embodiments, provided herein are methods of treating NSCLC in a subject, who has previously received an anti-PD-1 or anti-PD-L1 therapy, by administering an effective amount of a multi-specific binding protein, or a pharmaceutical formulation thereof. The multi-specific binding protein incorporates: a first polypeptide with the amino acid sequence of SEQ ID NO:167; (b) a second polypeptide with the amino acid sequence of SEQ ID NO:164; and (c) a third polypeptide with the amino acid sequence of SEQ ID NO:165.

In some embodiments, pharmaceutical formulations suitable for use in treating NSCLC in a subject, who has previously received an anti-PD-1 or anti-PD-L1 therapy, have 5 mg/mL to 50 mg/mL of the multi-specific binding protein (e.g., 5 mg/mL to 50 mg/mL, 5 mg/mL to 45 mg/mL, 5 mg/mL to 40 mg/mL, 5 mg/mL to 35 mg/mL, 5 mg/mL to 30 mg/mL, 5 mg/mL to 25 mg/mL, 5 mg/mL to 20 mg/mL, 5 mg/mL to 15 mg/mL, 5 mg/mL to 10 mg/mL, 10 mg/mL to 50 mg/mL, 15 mg/mL to 50 mg/mL, 20 mg/mL to 50 mg/mL, 25 mg/mL to 50 mg/mL, 30 mg/mL to 50 mg/mL, 35 mg/mL to 50 mg/mL, 40 mg/mL to 50 mg/mL, 45 mg/mL to 50 mg/mL, or 15 mg/mL), and also include: 15 mM to 25 mM citrate (e.g., 15 mM to 25 mM, 16 mM to 25 mM, 17 mM to 25 mM, 18 mM to 25 mM, 19 mM to 25 mM, 20 mM to 25 mM, 21 mM to 25 mM, 22 mM to 25 mM, 23 mM to 25 mM, 24 mM to 25 mM, 15 mM to 24 mM, 15 mM to 23 mM, 15 mM to 22 mM, 15 mM to 23 mM, 15 mM to 22 mM, 15 mM to 21 mM, 15 mM to 20 mM, 15 mM to 19 mM, 15 mM to 18 mM, 15 mM to 17 mM, 15 mM to 16 mM, or about 20 mM), 4% to 8% (w/v) mannitol (e.g., 4% to 8% (w/v), 4% to 7% (w/v), 4% to 6% (w/v), 4% to 5% (w/v), 5% to 8% (w/v), 6% to 8% (w/v), 7% to 8% (w/v), or about 6% (w/v)); and 0.005% to 0.05% (w/v) polysorbate 80 (e.g., 0.005% to 0.05% (w/v), 0.005% to 0.04% (w/v), 0.005% to 0.03% (w/v), 0.005% to 0.02% (w/v), 0.005% to 0.01% (w/v), 0.006% to 0.05% (w/v), 0.007% to 0.05% (w/v), 0.008% to 0.05% (w/v), 0.009% to 0.05% (w/v), 0.01% to 0.05% (w/v), 0.02% to 0.05% (w/v), 0.03% to 0.05% (w/v), 0.04% to 0.05% (w/v), or 0.01% (w/v)), at pH 6.2 to 6.8 (e.g., 6.2 to 6.8, 6.2 to 6.7, 6.2 to 6.6, 6.2 to 6.5, 6.2 to 6.4, 6.2 to 6.3, 6.3 to 6.8, 6.4 to 6.8, 6.5 to 6.8, 6.6 to 6.8, 6.7 to 6.8, or about 6.5). In some embodiments, pharmaceutical formulations suitable for use in treating NSCLC in a subject, who has previously received an anti-PD-1 or anti-PD-L1 therapy, include about 15 mg/mL of the multi-specific binding protein, and also include: about 20 mM citrate, about 6% (w/v) mannitol; and about 0.01% (w/v) polysorbate 80, at about pH 6.5.

In some embodiments, provided herein are methods for treating NSCLC in a subject, where the subject:
has histologically confirmed NSCLC meeting stage criteria for stage IIIB, stage IV, or recurrent disease;
does not have squamous histology;
has recurrent or progressive disease during or after platinum doublet-based chemotherapy or has recurrent or progressive disease within 6 months after completing platinum-based chemotherapy for local disease
has received and progressed on or after anti-PD-1/PD-L1 therapy;
has known status for actionable mutations (EGFR, ALK, ROS1, RET, etc.), and if actionable mutations are present, must have received and progressed on, have been intolerant to, or not have been a candidate for standard tyrosine kinase inhibitors (TKIs);
has adequate hematological function defined by WBC count>3×109/L, with ANC>1.5×109/L, lymphocyte count>0.5×109/L, platelet count>75×109/L, and hemoglobin>9 g/dL;
has adequate hepatic function defined by a total bilirubin level<1.5× the ULN, an AST level<2.5×ULN, and an ALT level<2.5 ULN, or, for patients with documented metastatic disease to the liver, AST and ALT levels<5×ULN; and/or
has adequate renal function defined by an estimated creatinine clearance>50 mL/min according to the Cockcroft-Gault formula or >30 mL/min.

In some embodiments, provided herein are methods of treating esophageal adenocarcinoma in a subject by administering an effective amount of a multi-specific binding protein, or a pharmaceutical formulation thereof. The multi-specific binding protein incorporates: a first antigen-binding site that binds NKG2D; a second antigen-binding site that binds EGFR; and an antibody Fc domain or a portion thereof sufficient to bind CD16, or a third antigen-binding site that binds CD16.

In some embodiments, provided herein are methods of treating esophageal adenocarcinoma in a subject by administering an effective amount of a multi-specific binding protein, or a pharmaceutical formulation thereof. The multi-specific binding protein incorporates: a first antigen binding site that binds NKG2D; a second antigen binding site that binds EGFR with (i) a VH having the CDR1, CDR2, and CDR3 sequences of SEQ ID NOs: 136, 157, and 138, respectively; and a VL having the CDR1, CDR2, and CDR3 sequences of SEQ ID NOs: 140, 141, and 151, respectively; (ii) a VH having the CDR1, CDR2, and CDR3 sequences of SEQ ID NOs: 136, 146, and 138, respectively; and a VL having the CDR1, CDR2, and CDR3 sequences of SEQ ID NOs: 140, 141, and 142, respectively; (iii) a VH having the CDR1, CDR2, and CDR3 sequences of SEQ ID NOs: 136, 146, and 138, respectively; and a VL having the CDR1, CDR2, and CDR3 sequences of SEQ ID NOs: 140, 141, and 151, respectively; (iv) a VH having the CDR1, CDR2, and CDR3 sequences of SEQ ID NOs: 136, 137, and 138, respectively; and a VL having the CDR1, CDR2, and CDR3 sequences of SEQ ID NOs: 140, 141, and 151, respectively; or (v) a VH having the CDR1, CDR2, and CDR3 sequences of SEQ ID NOs: 136, 137, and 138, respectively; and a VL having the CDR1, CDR2, and CDR3 sequences of SEQ ID NOs: 140, 141, and 142, respectively; and an antibody Fc domain or a portion thereof sufficient to bind CD16, or a third antigen-binding site that binds CD16.

In some embodiments, provided herein are methods of treating esophageal adenocarcinoma in a subject by administering an effective amount of a multi-specific binding protein, or a pharmaceutical formulation thereof. The multi-specific binding protein incorporates: a first antigen binding site that binds NKG2D with a VH having the CDR1, CDR2, and CDR3 sequences of SEQ ID NOs: 81, 82, and 97 or 112, respectively; and a VL having the CDR1, CDR2, and CDR3 sequences of SEQ ID NOs: 86, 77, and 87, respectively; a second antigen binding site that binds EGFR with (i) a VH having the CDR1, CDR2, and CDR3 sequences of SEQ ID NOs: 136, 157, and 138, respectively; and a VL having the CDR1, CDR2, and CDR3 sequences of SEQ ID NOs: 140, 141, and 151, respectively; (ii) a VH having the CDR1, CDR2, and CDR3 sequences of SEQ ID NOs: 136, 146, and 138, respectively; and a VL having the CDR1, CDR2, and CDR3 sequences of SEQ ID NOs: 140, 141, and 142, respectively; (iii) a VH having the CDR1, CDR2, and CDR3 sequences of SEQ ID NOs: 136, 146, and 138, respectively; and a VL having the CDR1, CDR2, and CDR3 sequences of SEQ ID NOs: 140, 141, and 151, respectively; (iv) a VH having the CDR1, CDR2, and CDR3 sequences of SEQ ID NOs: 136, 137, and 138, respectively; and a VL having the CDR1, CDR2, and CDR3 sequences of SEQ ID NOs: 140, 141, and 151, respectively; or (v) a VH having the CDR1, CDR2, and CDR3 sequences of SEQ ID NOs: 136, 137, and 138, respectively; and a VL having the CDR1, CDR2, and CDR3 sequences of SEQ ID NOs: 140, 141, and 142, respectively; and an antibody Fe domain or a portion thereof sufficient to bind CD16, or a third antigen-binding site that binds CD16.

In some embodiments, provided herein are methods of treating esophageal adenocarcinoma in a subject by administering an effective amount of a multi-specific binding protein, or a pharmaceutical formulation thereof. The multi-specific binding protein incorporates: a first polypeptide with the amino acid sequence of SEQ ID NO:167; (b) a second polypeptide with the amino acid sequence of SEQ ID NO:164; and (c) a third polypeptide with the amino acid sequence of SEQ ID NO:165.

In some embodiments, pharmaceutical formulations suitable for use in treating esophageal adenocarcinoma in a subject have 5 mg/mL to 50 mg/mL of the multi-specific binding protein (e.g., 5 mg/mL to 50 mg/mL, 5 mg/mL to 45 mg/mL, 5 mg/mL to 40 mg/mL, 5 mg/mL to 35 mg/mL, 5 mg/mL to 30 mg/mL, 5 mg/mL to 25 mg/mL, 5 mg/mL to 20 mg/mL, 5 mg/mL to 15 mg/mL, 5 mg/mL to 10 mg/mL, 10 mg/mL to 50 mg/mL, 15 mg/mL to 50 mg/mL, 20 mg/mL to 50 mg/mL, 25 mg/mL to 50 mg/mL, 30 mg/mL to 50 mg/mL, 35 mg/mL to 50 mg/mL, 40 mg/mL to 50 mg/mL, 45 mg/mL to 50 mg/mL, or 15 mg/mL), and also include: 15 mM to 25 mM citrate (e.g., 15 mM to 25 mM, 16 mM to 25 mM, 17 mM to 25 mM, 18 mM to 25 mM, 19 mM to 25 mM, 20 mM to 25 mM, 21 mM to 25 mM, 22 mM to 25 mM, 23 mM to 25 mM, 24 mM to 25 mM, 15 mM to 24 mM, 15 mM to 23 mM, 15 mM to 22 mM, 15 mM to 23 mM, 15 mM to 22 mM, 15 mM to 21 mM, 15 mM to 20 mM, 15 mM to 19 mM, 15 mM to 18 mM, 15 mM to 17 mM, 15 mM to 16 mM, or about 20 mM), 4% to 8% (w/v) mannitol (e.g., 4% to 8% (w/v), 4% to 7% (w/v), 4% to 6% (w/v), 4% to 5% (w/v), 5% to 8% (w/v), 6% to 8% (w/v), 7% to 8% (w/v), or about 6% (w/v)); and 0.005% to 0.05% (w/v) polysorbate 80 (e.g., 0.005% to 0.05% (w/v), 0.005% to 0.04% (w/v), 0.005% to 0.03% (w/v), 0.005% to 0.02% (w/v), 0.005% to 0.01% (w/v), 0.006% to 0.05% (w/v), 0.007% to 0.05% (w/v), 0.008% to 0.05% (w/v), 0.009% to 0.05% (w/v), 0.01% to 0.05% (w/v), 0.02% to 0.05% (w/v), 0.03% to 0.05% (w/v), 0.04% to 0.05% (w/v), or 0.01% (w/v)), at pH 6.2 to 6.8 (e.g., 6.2 to 6.8, 6.2 to 6.7, 6.2 to 6.6, 6.2 to 6.5, 6.2 to 6.4, 6.2 to 6.3, 6.3 to 6.8, 6.4 to 6.8, 6.5 to 6.8, 6.6 to 6.8, 6.7 to 6.8, or about 6.5). In some embodiments, pharmaceutical formulations suitable for use in treating esophageal adenocarcinoma in a subject include about 15 mg/mL of the multi-specific binding protein, and also include: about 20 mM citrate, about 6% (w/v) mannitol; and about 0.01% (w/v) polysorbate 80, at about pH 6.5.

In some embodiments, provided herein are methods of treating triple-negative breast cancer in a subject by administering an effective amount of a multi-specific binding protein, or a pharmaceutical formulation thereof. The multi-specific binding protein incorporates: a first antigen-binding site that binds NKG2D; a second antigen-binding site that binds EGFR; and an antibody Fc domain or a portion thereof sufficient to bind CD16, or a third antigen-binding site that binds CD16.

In some embodiments, provided herein are methods of treating triple-negative breast cancer in a subject by administering an effective amount of a multi-specific binding protein, or a pharmaceutical formulation thereof. The multi-specific binding protein incorporates: a first antigen binding site that binds NKG2D; a second antigen binding site that binds EGFR with (i) a VH having the CDR1, CDR2, and CDR3 sequences of SEQ ID NOs: 136, 157, and 138, respectively; and a VL having the CDR1, CDR2, and CDR3 sequences of SEQ ID NOs: 140, 141, and 151, respectively; (ii) a VH having the CDR1, CDR2, and CDR3 sequences of SEQ ID NOs: 136, 146, and 138, respectively; and a VL having the CDR1, CDR2, and CDR3 sequences of SEQ ID NOs: 140, 141, and 142, respectively; (iii) a VH having the CDR1, CDR2, and CDR3 sequences of SEQ ID NOs: 136, 146, and 138, respectively; and a VL having the CDR1, CDR2, and CDR3 sequences of SEQ ID NOs: 140, 141, and 151, respectively; (iv) a VH having the CDR1, CDR2, and CDR3 sequences of SEQ ID NOs: 136, 137, and 138, respectively; and a VL having the CDR1, CDR2, and CDR3 sequences of SEQ ID NOs: 140, 141, and 151, respectively; or (v) a VH having the CDR1, CDR2, and CDR3 sequences of SEQ ID NOs: 136, 137, and 138, respectively; and a VL having the CDR1, CDR2, and CDR3 sequences of SEQ ID NOs: 140, 141, and 142, respectively; and an antibody Fc domain or a portion thereof sufficient to bind CD16, or a third antigen-binding site that binds CD16.

In some embodiments, provided herein are methods of treating triple-negative breast cancer in a subject by administering an effective amount of a multi-specific binding protein, or a pharmaceutical formulation thereof. The multi-specific binding protein incorporates: a first antigen binding site that binds NKG2D with a VH having the CDR1, CDR2, and CDR3 sequences of SEQ ID NOs: 81, 82, and 97 or 112, respectively; and a VL having the CDR1, CDR2, and CDR3 sequences of SEQ ID NOs: 86, 77, and 87, respectively; a second antigen binding site that binds EGFR with (i) a VH having the CDR1, CDR2, and CDR3 sequences of SEQ ID NOs: 136, 157, and 138, respectively; and a VL having the CDR1, CDR2, and CDR3 sequences of SEQ ID NOs: 140, 141, and 151, respectively; (ii) a VH having the CDR1, CDR2, and CDR3 sequences of SEQ ID NOs: 136, 146, and 138, respectively; and a VL having the CDR1, CDR2, and CDR3 sequences of SEQ ID NOs: 140, 141, and 142, respectively; (iii) a VH having the CDR1, CDR2, and CDR3 sequences of SEQ ID NOs: 136, 146, and 138, respectively; and a VL having the CDR1, CDR2, and CDR3 sequences of SEQ ID NOs: 140, 141, and 151, respectively; (iv) a VH having the CDR1, CDR2, and CDR3 sequences of SEQ ID NOs: 136, 137, and 138, respectively; and a VL having the CDR1, CDR2, and CDR3 sequences of SEQ ID NOs: 140, 141, and 151, respectively; or (v) a VH having the CDR1, CDR2, and CDR3 sequences of SEQ ID NOs: 136, 137, and 138, respectively; and a VL having the CDR1, CDR2, and CDR3 sequences of SEQ ID NOs: 140, 141, and 142, respectively; and an antibody Fc domain or a portion thereof sufficient to bind CD16, or a third antigen-binding site that binds CD16.

In some embodiments, provided herein are methods of treating triple-negative breast cancer in a subject by administering an effective amount of a multi-specific binding protein, or a pharmaceutical formulation thereof. The multi-specific binding protein incorporates: a first polypeptide with the amino acid sequence of SEQ ID NO:167; (b) a second polypeptide with the amino acid sequence of SEQ ID NO:164; and (c) a third polypeptide with the amino acid sequence of SEQ ID NO:165.

In some embodiments, pharmaceutical formulations described in the present disclosure are suitable for use in treating triple-negative breast cancer in a subject, and have 5 mg/mL to 50 mg/mL of the multi-specific binding protein (e.g., 5 mg/mL to 50 mg/mL, 5 mg/mL to 45 mg/mL, 5 mg/mL to 40 mg/mL, 5 mg/mL to 35 mg/mL, 5 mg/mL to 30 mg/mL, 5 mg/mL to 25 mg/mL, 5 mg/mL to 20 mg/mL, 5 mg/mL to 15 mg/mL, 5 mg/mL to 10 mg/mL, 10 mg/mL to 50 mg/mL, 15 mg/mL to 50 mg/mL, 20 mg/mL to 50 mg/mL, 25 mg/mL to 50 mg/mL, 30 mg/mL to 50 mg/mL, 35 mg/mL to 50 mg/mL, 40 mg/mL to 50 mg/mL, 45 mg/mL to 50 mg/mL, or 15 mg/mL), 15 mM to 25 mM citrate (e.g., 15 mM to 25 mM, 16 mM to 25 mM, 17 mM to 25 mM, 18 mM to 25 mM, 19 mM to 25 mM, 20 mM to 25 mM, 21 mM to 25 mM, 22 mM to 25 mM, 23 mM to 25 mM, 24 mM to 25 mM, 15 mM to 24 mM, 15 mM to 23 mM, 15 mM to 22 mM, 15 mM to 23 mM, 15 mM to 22 mM, 15 mM to 21 mM, 15 mM to 20 mM, 15 mM to 19 mM, 15 mM to 18 mM, 15 mM to 17 mM, 15 mM to 16 mM, or about 20 mM), 4% to 8% (w/v) mannitol (e.g., 4% to 8% (w/v), 4% to 7% (w/v), 4% to 6% (w/v), 4% to 5% (w/v), 5% to 8% (w/v), 6% to 8% (w/v), 7% to 8% (w/v), or about 6% (w/v)); and 0.005% to 0.05% (w/v) polysorbate 80 (e.g., 0.005% to 0.05% (w/v), 0.005% to 0.04% (w/v), 0.005% to 0.03% (w/v), 0.005% to 0.02% (w/v), 0.005% to 0.01% (w/v), 0.006% to 0.05% (w/v), 0.007% to 0.05% (w/v), 0.008% to 0.05% (w/v), 0.009% to 0.05% (w/v), 0.01% to 0.05% (w/v), 0.02% to 0.05% (w/v), 0.03% to 0.05% (w/v), 0.04% to 0.05% (w/v), or 0.01% (w/v)), at pH 6.2 to 6.8 (e.g., 6.2 to 6.8, 6.2 to 6.7, 6.2 to 6.6, 6.2 to 6.5, 6.2 to 6.4, 6.2 to 6.3, 6.3 to 6.8, 6.4 to 6.8, 6.5 to 6.8, 6.6 to 6.8, 6.7 to 6.8, or about 6.5). In some embodiments, pharmaceutical formulations, suitable for use in treating triple-negative breast cancer in a subject, include about 15 mg/mL of the multi-specific binding protein, and also include: about 20 mM citrate, about 6% (w/v) mannitol; and about 0.01% (w/v) polysorbate 80, at about pH 6.5.

As described herein, in various embodiments, are methods of treating renal cell carcinoma in a subject by administering an effective amount of a multi-specific binding protein, or a pharmaceutical formulation thereof. The multi-specific binding protein incorporates: a first antigen-binding site that binds NKG2D; a second antigen-binding site that binds EGFR; and an antibody Fc domain or a portion thereof sufficient to bind CD16, or a third antigen-binding site that binds CD16.

As described herein, in various embodiments, are methods of treating renal cell carcinoma in a subject by administering an effective amount of a multi-specific binding protein, or a pharmaceutical formulation thereof. The multi-specific binding protein incorporates: a first antigen binding site that binds NKG2D; a second antigen binding site that binds EGFR with (i) a VH having the CDR1, CDR2, and CDR3 sequences of SEQ ID NOs: 136, 157, and 138, respectively; and a VL having the CDR1, CDR2, and CDR3 sequences of SEQ ID NOs: 140, 141, and 151, respectively; (ii) a VH having the CDR1, CDR2, and CDR3 sequences of SEQ ID NOs: 136, 146, and 138, respectively; and a VL having the CDR1, CDR2, and CDR3 sequences of SEQ ID NOs: 140, 141, and 142, respectively; (iii) a VH having the CDR1, CDR2, and CDR3 sequences of SEQ ID NOs: 136, 146, and 138, respectively; and a VL having the CDR1, CDR2, and CDR3 sequences of SEQ ID NOs: 140, 141, and 151, respectively; (iv) a VH having the CDR1, CDR2, and CDR3 sequences of SEQ ID NOs: 136, 137, and 138, respectively; and a VL having the CDR1, CDR2, and CDR3 sequences of SEQ ID NOs: 140, 141, and 151, respectively; or (v) a VH having the CDR1, CDR2, and CDR3 sequences of SEQ ID NOs: 136, 137, and 138, respectively; and a VL having the CDR1, CDR2, and CDR3 sequences of SEQ ID NOs: 140, 141, and 142, respectively; and an antibody Fc domain or a portion thereof sufficient to bind CD16, or a third antigen-binding site that binds CD16.

As described herein, in various embodiments, are methods of treating renal cell carcinoma in a subject by administering an effective amount of a multi-specific binding protein, or a pharmaceutical formulation thereof. The multi-specific binding protein incorporates: a first antigen binding site that binds NKG2D with a VH having the CDR1, CDR2, and CDR3 sequences of SEQ ID NOs: 81, 82, and 97 or 112, respectively; and a VL having the CDR1, CDR2, and CDR3 sequences of SEQ ID NOs: 86, 77, and 87, respectively; a second antigen binding site that binds EGFR with (i) a VH having the CDR1, CDR2, and CDR3 sequences of SEQ ID NOs: 136, 157, and 138, respectively; and a VL having the CDR1, CDR2, and CDR3 sequences of SEQ ID NOs: 140, 141, and 151, respectively; (ii) a VH having the CDR1, CDR2, and CDR3 sequences of SEQ ID NOs: 136, 146, and 138, respectively; and a VL having the CDR1, CDR2, and CDR3 sequences of SEQ ID NOs: 140, 141, and 142, respectively; (iii) a VH having the CDR1, CDR2, and CDR3 sequences of SEQ ID NOs: 136, 146, and 138, respectively; and a VL having the CDR1, CDR2, and CDR3 sequences of SEQ ID NOs: 140, 141, and 151, respectively; (iv) a VH having the CDR1, CDR2, and CDR3 sequences of SEQ ID NOs: 136, 137, and 138, respectively; and a VL having the CDR1, CDR2, and CDR3 sequences of SEQ ID NOs: 140, 141, and 151, respectively; or (v) a VH having the CDR1, CDR2, and CDR3 sequences of SEQ ID NOs: 136, 137, and 138, respectively; and a VL having the CDR1, CDR2, and CDR3 sequences of SEQ ID NOs: 140, 141, and 142, respectively; and an antibody Fc domain or a portion thereof sufficient to bind CD16, or a third antigen-binding site that binds CD16.

As described herein, in various embodiments, are methods of treating renal cell carcinoma in a subject by administering an effective amount of a multi-specific binding protein, or a pharmaceutical formulation thereof. The multi-specific binding protein incorporates: a first polypeptide with the amino acid sequence of SEQ ID NO:167; (b) a second polypeptide with the amino acid sequence of SEQ ID NO:164; and (c) a third polypeptide with the amino acid sequence of SEQ ID NO:165.

In some embodiments, pharmaceutical formulations described in the present disclosure are suitable for use in treating renal cell carcinoma in a subject, and have 5 mg/mL to 50 mg/mL of the multi-specific binding protein (e.g., 5 mg/mL to 50 mg/mL, 5 mg/mL to 45 mg/mL, 5 mg/mL to 40 mg/mL, 5 mg/mL to 35 mg/mL, 5 mg/mL to 30 mg/mL, 5 mg/mL to 25 mg/mL, 5 mg/mL to 20 mg/mL, 5 mg/mL to 15 mg/mL, 5 mg/mL to 10 mg/mL, 10 mg/mL to 50 mg/mL, 15 mg/mL to 50 mg/mL, 20 mg/mL to 50 mg/mL, 25 mg/mL to 50 mg/mL, 30 mg/mL to 50 mg/mL, 35 mg/mL to 50 mg/mL, 40 mg/mL to 50 mg/mL, 45 mg/mL to 50 mg/mL, or 15 mg/mL), 15 mM to 25 mM citrate (e.g., 15 mM to 25 mM, 16 mM to 25 mM, 17 mM to 25 mM, 18 mM to 25 mM, 19 mM to 25 mM, 20 mM to 25 mM, 21 mM to 25 mM, 22 mM to 25 mM, 23 mM to 25 mM, 24 mM to 25 mM, 15 mM to 24 mM, 15 mM to 23 mM, 15 mM to 22 mM, 15 mM to 23 mM, 15 mM to 22 mM, 15 mM to 21 mM, 15 mM to 20 mM, 15 mM to 19 mM, 15 mM to 18 mM, 15 mM to 17 mM, 15 mM to 16 mM, or about 20 mM), 4% to 8% (w/v) mannitol (e.g., 4% to 8% (w/v), 4% to 7% (w/v), 4% to 6% (w/v), 4% to 5% (w/v), 5% to 8% (w/v), 6% to 8% (w/v), 7% to 8% (w/v), or about 6% (w/v)); and 0.005% to 0.05% (w/v) polysorbate 80 (e.g., 0.005% to 0.05% (w/v), 0.005% to 0.04% (w/v), 0.005% to 0.03% (w/v), 0.005% to 0.02% (w/v), 0.005% to 0.01% (w/v), 0.006% to 0.05% (w/v), 0.007% to 0.05% (w/v), 0.008% to 0.05% (w/v), 0.009% to 0.05% (w/v), 0.01% to 0.05% (w/v), 0.02% to 0.05% (w/v), 0.03% to 0.05% (w/v), 0.04% to 0.05% (w/v), or 0.01% (w/v)), at pH 6.2 to 6.8 (e.g., 6.2 to 6.8, 6.2 to 6.7, 6.2 to 6.6, 6.2 to 6.5, 6.2 to 6.4, 6.2 to 6.3, 6.3 to 6.8, 6.4 to 6.8, 6.5 to 6.8, 6.6 to 6.8, 6.7 to 6.8, or about 6.5). In some embodiments, pharmaceutical formulations, suitable for use in treating renal cell carcinoma in a subject, include about 15 mg/mL of the multi-specific binding protein, and also include: about 20 mM citrate, about 6% (w/v) mannitol; and about 0.01% (w/v) polysorbate 80, at about pH 6.5.

As described herein, in various embodiments, are methods of treating gastric cancer in a subject by administering an effective amount of a multi-specific binding protein, or a pharmaceutical formulation thereof. The multi-specific binding protein incorporates: a first antigen-binding site that binds NKG2D; a second antigen-binding site that binds EGFR; and an antibody Fc domain or a portion thereof sufficient to bind CD16, or a third antigen-binding site that binds CD16.

As described herein, in various embodiments, are methods of treating gastric cancer in a subject by administering an effective amount of a multi-specific binding protein, or a pharmaceutical formulation thereof. The multi-specific binding protein incorporates: a first antigen binding site that binds NKG2D; a second antigen binding site that binds EGFR with (i) a VH having the CDR1, CDR2, and CDR3 sequences of SEQ ID NOs: 136, 157, and 138, respectively; and a VL having the CDR1, CDR2, and CDR3 sequences of SEQ ID NOs: 140, 141, and 151, respectively; (ii) a VH having the CDR1, CDR2, and CDR3 sequences of SEQ ID NOs: 136, 146, and 138, respectively; and a VL having the CDR1, CDR2, and CDR3 sequences of SEQ ID NOs: 140, 141, and 142, respectively; (iii) a VH having the CDR1, CDR2, and CDR3 sequences of SEQ ID NOs: 136, 146, and 138, respectively; and a VL having the CDR1, CDR2, and CDR3 sequences of SEQ ID NOs: 140, 141, and 151, respectively; (iv) a VH having the CDR1, CDR2, and CDR3 sequences of SEQ ID NOs: 136, 137, and 138, respectively; and a VL having the CDR1, CDR2, and CDR3 sequences of SEQ ID NOs: 140, 141, and 151, respectively; or (v) a VH having the CDR1, CDR2, and CDR3 sequences of SEQ ID NOs: 136, 137, and 138, respectively; and a VL having the CDR1, CDR2, and CDR3 sequences of SEQ ID NOs: 140, 141, and 142, respectively; and an antibody Fc domain or a portion thereof sufficient to bind CD16, or a third antigen-binding site that binds CD16.

As described herein, in various embodiments, are methods of treating gastric cancer in a subject by administering an effective amount of a multi-specific binding protein, or a pharmaceutical formulation thereof. The multi-specific binding protein incorporates: a first antigen binding site that binds NKG2D with a VH having the CDR1, CDR2, and CDR3 sequences of SEQ ID NOs: 81, 82, and 97 or 112, respectively; and a VL having the CDR1, CDR2, and CDR3 sequences of SEQ ID NOs: 86, 77, and 87, respectively; a second antigen binding site that binds EGFR with (i) a VH having the CDR1, CDR2, and CDR3 sequences of SEQ ID NOs: 136, 157, and 138, respectively; and a VL having the CDR1, CDR2, and CDR3 sequences of SEQ ID NOs: 140, 141, and 151, respectively; (ii) a VH having the CDR1, CDR2, and CDR3 sequences of SEQ ID NOs: 136, 146, and 138, respectively; and a VL having the CDR1, CDR2, and CDR3 sequences of SEQ ID NOs: 140, 141, and 142, respectively; (iii) a VH having the CDR1, CDR2, and CDR3 sequences of SEQ ID NOs: 136, 146, and 138, respectively; and a VL having the CDR1, CDR2, and CDR3 sequences of SEQ ID NOs: 140, 141, and 151, respectively; (iv) a VH having the CDR1, CDR2, and CDR3 sequences of SEQ ID NOs: 136, 137, and 138, respectively; and a VL having the CDR1, CDR2, and CDR3 sequences of SEQ ID NOs: 140, 141, and 151, respectively; or (v) a VH having the CDR1, CDR2, and CDR3 sequences of SEQ ID NOs: 136, 137, and 138, respectively; and a VL having the CDR1, CDR2, and CDR3 sequences of SEQ ID NOs: 140, 141, and 142, respectively; and an antibody Fe domain or a portion thereof sufficient to bind CD16, or a third antigen-binding site that binds CD16.

As described herein, in various embodiments, are methods of treating gastric cancer in a subject by administering an effective amount of a multi-specific binding protein, or a pharmaceutical formulation thereof. The multi-specific binding protein incorporates: a first polypeptide with the amino acid sequence of SEQ ID NO:167; (b) a second polypeptide with the amino acid sequence of SEQ ID NO:164; and (c) a third polypeptide with the amino acid sequence of SEQ ID NO:165.

In some embodiments, pharmaceutical formulations described in the present disclosure are suitable for use in treating gastric cancer in a subject, and have 5 mg/mL to 50 mg/mL of the multi-specific binding protein (e.g., 5 mg/mL to 50 mg/mL, 5 mg/mL to 45 mg/mL, 5 mg/mL to 40 mg/mL, 5 mg/mL to 35 mg/mL, 5 mg/mL to 30 mg/mL, 5 mg/mL to 25 mg/mL, 5 mg/mL to 20 mg/mL, 5 mg/mL to 15 mg/mL, 5 mg/mL to 10 mg/mL, 10 mg/mL to 50 mg/mL, 15 mg/mL to 50 mg/mL, 20 mg/mL to 50 mg/mL, 25 mg/mL to 50 mg/mL, 30 mg/mL to 50 mg/mL, 35 mg/mL to 50 mg/mL, 40 mg/mL to 50 mg/mL, 45 mg/mL to 50 mg/mL, or 15 mg/mL), 15 mM to 25 mM citrate (e.g., 15 mM to 25 mM, 16 mM to 25 mM, 17 mM to 25 mM, 18 mM to 25 mM, 19 mM to 25 mM, 20 mM to 25 mM, 21 mM to 25 mM, 22 mM to 25 mM, 23 mM to 25 mM, 24 mM to 25 mM, 15 mM to 24 mM, 15 mM to 23 mM, 15 mM to 22 mM, 15 mM to 23 mM, 15 mM to 22 mM, 15 mM to 21 mM, 15 mM to 20 mM, 15 mM to 19 mM, 15 mM to 18 mM, 15 mM to 17 mM, 15 mM to 16 mM, or about 20 mM), 4% to 8% (w/v) mannitol (e.g., 4% to 8% (w/v), 4% to 7% (w/v), 4% to 6% (w/v), 4% to 5% (w/v), 5% to 8% (w/v), 6% to 8% (w/v), 7% to 8% (w/v), or about 6% (w/v)); and 0.005% to 0.05% (w/v) polysorbate 80 (e.g., 0.005% to 0.05% (w/v), 0.005% to 0.04% (w/v), 0.005% to 0.03% (w/v), 0.005% to 0.02% (w/v), 0.005% to 0.01% (w/v), 0.006% to 0.05% (w/v), 0.007% to 0.05% (w/v), 0.008% to 0.05% (w/v), 0.009% to 0.05% (w/v), 0.01% to 0.05% (w/v), 0.02% to 0.05% (w/v), 0.03% to 0.05% (w/v), 0.04% to 0.05% (w/v), or 0.01% (w/v)), at pH 6.2 to 6.8 (e.g., 6.2 to 6.8, 6.2 to 6.7, 6.2 to 6.6, 6.2 to 6.5, 6.2 to 6.4, 6.2 to 6.3, 6.3 to 6.8, 6.4 to 6.8, 6.5 to 6.8, 6.6 to 6.8, 6.7 to 6.8, or about 6.5). In some embodiments, pharmaceutical formulations, suitable for use in treating gastric cancer in a subject, include about 15 mg/mL of the multi-specific binding protein, and also include: about 20 mM citrate, about 6% (w/v) mannitol; and about 0.01% (w/v) polysorbate 80, at about pH 6.5.

As described herein, in various embodiments, are methods of treating pancreatic cancer in a subject by administering an effective amount of a multi-specific binding protein, or a pharmaceutical formulation thereof. The multi-specific binding protein incorporates: a first antigen-binding site that binds NKG2D; a second antigen-binding site that binds EGFR; and an antibody Fc domain or a portion thereof sufficient to bind CD16, or a third antigen-binding site that binds CD16.

As described herein, in various embodiments, are methods of treating pancreatic cancer in a subject by administering an effective amount of a multi-specific binding protein, or a pharmaceutical formulation thereof. The multi-specific binding protein incorporates: a first antigen binding site that binds NKG2D; a second antigen binding site that binds EGFR with (i) a VH having the CDR1, CDR2, and CDR3 sequences of SEQ ID NOs: 136, 157, and 138, respectively; and a VL having the CDR1, CDR2, and CDR3 sequences of SEQ ID NOs: 140, 141, and 151, respectively; (ii) a VH having the CDR1, CDR2, and CDR3 sequences of SEQ ID NOs: 136, 146, and 138, respectively; and a VL having the CDR1, CDR2, and CDR3 sequences of SEQ ID NOs: 140, 141, and 142, respectively; (iii) a VH having the CDR1, CDR2, and CDR3 sequences of SEQ ID NOs: 136, 146, and 138, respectively; and a VL having the CDR1, CDR2, and CDR3 sequences of SEQ ID NOs: 140, 141, and 151, respectively; (iv) a VH having the CDR1, CDR2, and CDR3 sequences of SEQ ID NOs: 136, 137, and 138, respectively; and a VL having the CDR1, CDR2, and CDR3 sequences of SEQ ID NOs: 140, 141, and 151, respectively; or (v) a VH having the CDR1, CDR2, and CDR3 sequences of SEQ ID NOs: 136, 137, and 138, respectively; and a VL having the CDR1, CDR2, and CDR3 sequences of SEQ ID NOs: 140, 141, and 142, respectively; and an antibody Fc domain or a portion thereof sufficient to bind CD16, or a third antigen-binding site that binds CD16.

As described herein, in various embodiments, are methods of treating pancreatic cancer in a subject by administering an effective amount of a multi-specific binding protein, or a pharmaceutical formulation thereof. The multi-specific binding protein incorporates: a first antigen binding site that binds NKG2D with a VH having the CDR1, CDR2, and CDR3 sequences of SEQ ID NOs: 81, 82, and 97 or 112, respectively; and a VL having the CDR1, CDR2, and CDR3 sequences of SEQ ID NOs: 86, 77, and 87, respectively; a second antigen binding site that binds EGFR with (i) a VH having the CDR1, CDR2, and CDR3 sequences of SEQ ID NOs: 136, 157, and 138, respectively; and a VL having the CDR1, CDR2, and CDR3 sequences of SEQ ID NOs: 140, 141, and 151, respectively; (ii) a VH having the CDR1, CDR2, and CDR3 sequences of SEQ ID NOs: 136, 146, and 138, respectively; and a VL having the CDR1, CDR2, and CDR3 sequences of SEQ ID NOs: 140, 141, and 142, respectively; (iii) a VH having the CDR1, CDR2, and CDR3 sequences of SEQ ID NOs: 136, 146, and 138, respectively; and a VL having the CDR1, CDR2, and CDR3 sequences of SEQ ID NOs: 140, 141, and 151, respectively; (iv) a VH having the CDR1, CDR2, and CDR3 sequences of SEQ ID NOs: 136, 137, and 138, respectively; and a VL having the CDR1, CDR2, and CDR3 sequences of SEQ ID NOs: 140, 141, and 151, respectively; or (v) a VH having the CDR1, CDR2, and CDR3 sequences of SEQ ID NOs: 136, 137, and 138, respectively; and a VL having the CDR1, CDR2, and CDR3 sequences of SEQ ID NOs: 140, 141, and 142, respectively; and an antibody Fc domain or a portion thereof sufficient to bind CD16, or a third antigen-binding site that binds CD16.

As described herein, in various embodiments, are methods of treating pancreatic cancer in a subject by administering an effective amount of a multi-specific binding protein, or a pharmaceutical formulation thereof. The multi-specific binding protein incorporates: a first polypeptide with the amino acid sequence of SEQ ID NO:167; (b) a second polypeptide with the amino acid sequence of SEQ ID NO:164; and (c) a third polypeptide with the amino acid sequence of SEQ ID NO:165.

In some embodiments, pharmaceutical formulations described in the present disclosure are suitable for use in treating pancreatic cancer in a subject, and have 5 mg/mL to 50 mg/mL of the multi-specific binding protein (e.g., 5 mg/mL to 50 mg/mL, 5 mg/mL to 45 mg/mL, 5 mg/mL to 40 mg/mL, 5 mg/mL to 35 mg/mL, 5 mg/mL to 30 mg/mL, 5 mg/mL to 25 mg/mL, 5 mg/mL to 20 mg/mL, 5 mg/mL to 15 mg/mL, 5 mg/mL to 10 mg/mL, 10 mg/mL to 50 mg/mL, 15 mg/mL to 50 mg/mL, 20 mg/mL to 50 mg/mL, 25 mg/mL to 50 mg/mL, 30 mg/mL to 50 mg/mL, 35 mg/mL to 50 mg/mL, 40 mg/mL to 50 mg/mL, 45 mg/mL to 50 mg/mL, or 15 mg/mL), 15 mM to 25 mM citrate (e.g., 15 mM to 25 mM, 16 mM to 25 mM, 17 mM to 25 mM, 18 mM to 25 mM, 19 mM to 25 mM, 20 mM to 25 mM, 21 mM to 25 mM, 22 mM to 25 mM, 23 mM to 25 mM, 24 mM to 25 mM, 15 mM to 24 mM, 15 mM to 23 mM, 15 mM to 22 mM, 15 mM to 23 mM, 15 mM to 22 mM, 15 mM to 21 mM, 15 mM to 20 mM, 15 mM to 19 mM, 15 mM to 18 mM, 15 mM to 17 mM, 15 mM to 16 mM, or about 20 mM), 4% to 8% (w/v) mannitol (e.g., 4% to 8% (w/v), 4% to 7% (w/v), 4% to 6% (w/v), 4% to 5% (w/v), 5% to 8% (w/v), 6% to 8% (w/v), 7% to 8% (w/v), or about 6% (w/v)); and 0.005% to 0.05% (w/v) polysorbate 80 (e.g., 0.005% to 0.05% (w/v), 0.005% to 0.04% (w/v), 0.005% to 0.03% (w/v), 0.005% to 0.02% (w/v), 0.005% to 0.01% (w/v), 0.006% to 0.05% (w/v), 0.007% to 0.05% (w/v), 0.008% to 0.05% (w/v), 0.009% to 0.05% (w/v), 0.01% to 0.05% (w/v), 0.02% to 0.05% (w/v), 0.03% to 0.05% (w/v), 0.04% to 0.05% (w/v), or 0.01% (w/v)), at pH 6.2 to 6.8 (e.g., 6.2 to 6.8, 6.2 to 6.7, 6.2 to 6.6, 6.2 to 6.5, 6.2 to 6.4, 6.2 to 6.3, 6.3 to 6.8, 6.4 to 6.8, 6.5 to 6.8, 6.6 to 6.8, 6.7 to 6.8, or about 6.5). In some embodiments, pharmaceutical formulations, suitable for use in treating pancreatic cancer in a subject, include about 15 mg/mL of the multi-specific binding protein, and also include: about 20 mM citrate, about 6% (w/v) mannitol; and about 0.01% (w/v) polysorbate 80, at about pH 6.5.

In some embodiments, patients treated with a multi-specific binding protein or a pharmaceutical formulation thereof are cancer patients with the following prerequisites:
untreated with chemotherapy, radiotherapy (other than palliative bone-directed radiotherapy); no major surgery, or not received another investigational agent within 28 days before the start of treatment;
no concurrent anticancer treatment (e.g., cytoreductive therapy, radiotherapy [except for palliative bone-directed radiotherapy], immune therapy, or cytokine therapy [except for erythropoietin]), major surgery (excluding prior diagnostic biopsy), concurrent systemic therapy with steroids or other immunosuppressive agents, or use of any investigational drug within 28 days before the start of treatment.
no previous malignant disease other than the target malignancy within the last 3 years before start of treatment, with the exception of basal or squamous cell carcinoma of the skin, low-grade prostate cancer (Gleason score of <6), or cervical carcinoma in situ;
do not have brain metastases unless:
   CNS lesions are asymptomatic and previously treated,
   do not require ongoing steroid treatment daily for replacement for adrenal insufficiency (except oral steroids at a dose less than <10 mg of prednisone [or equivalent]),
   imaging demonstrates stability of disease 28 days from last treatment for CNS metastases.
have not received any organ transplantation, including autologous or allogeneic stem-cell transplantation;
have not had significant acute or chronic infection with hepatitis B virus (HBV) or hepatitis C virus (IICV), nor a historic positive test for human immunodeficiency virus (HIV);
have not had preexisting autoimmune disease (except for patients with vitiligo) needing treatment with systemic immunosuppressive agents for more than 28 days within the last 3 years, or clinically relevant immunodeficiencies (e.g., dysgammaglobulinemia or congenital immunodeficiencies);
have not had a known medical history of keratitis, ulcerative keratitis, or corneal perforation;
have not had known severe hypersensitivity reactions to monoclonal antibodies (>Grade 3 of the National Cancer Institute-Common Terminology Criteria for Adverse Events [NCI-CTCAE] v5.0), any history of anaphylaxis, or uncontrolled asthma (i.e., 3 or more features of partly controlled asthma);
have not had persisting toxicity related to prior therapy>Grade 1 NCI-CTCAE v5.0;
however, alopecia<Grade 2, endocrinopathies<Grade 2, and sensory neuropathy<Grade 2 is acceptable;
have not had received an anti-PD-1 or an anti-PD-L1 as a previous line of therapy, where they experienced either:
   a grade 3 or 4 drug-related toxicity during and attributed to treatment with the anti-PD-1 or anti-PD-L1; or
   a grade 2 drug-related toxicity that impacted either the lungs or the nervous system, caused by the administration of the anti-PD-1 or anti-PD-L1;
not be pregnant or lactating;
have not had known alcohol or drug abuse;
have not had serious cardiac illness or medical conditions;
have not had any other significant disease (e.g., inflammatory bowel disease).

In some embodiments, patients treated with a multi-specific binding protein or a pharmaceutical formulation thereof disclosed herein exhibit no adverse events after one, two, three, four, five, six, or seven weeks of treatment (e.g., weekly treatment). In some embodiments, patients treated with a multi-specific binding protein or a pharmaceutical formulation thereof disclosed herein exhibit no adverse events of grade 3 or higher after one, two, three, four, five, six, or seven weeks of treatment (e.g., weekly treatment).

The cancer to be treated can be characterized according to the presence of a particular antigen expressed on the surface of the cancer cell. In certain embodiments, the cancer cell can express one or more of the following in addition to EGFR: CD2, CD19, CD38, CD40, CD52, CD30, CD70, IGF1R, HER3/ERBB3, HER4/ERBB4, MUC1, TROP2, cMET, SLAMF7, PSCA, MICA, MICB, TRAILR1, TRAILR2, MAGE-A3, B7.1, B7.2, CTLA4, and PD1.

In some embodiments, provided herein are methods of treating cancer in a subject, where the subject:
is male or female aged≥18 years;
has histologically or cytologically proven locally advanced or metastatic solid tumors of epithelial origin for which the expression of EGFR has been reported in the literature or who carry an activating EGFR mutation, for which no standard therapy exists, or standard therapy has failed;
has evidence of objective disease, but does not necessarily have a measurable lesion
has adequate hematological function defined by white blood cell (WBC) count≥3×10⁹/L, with absolute neutrophil count (ANC)≥1.5×10⁹/L, lymphocyte count≥0.5×10⁹/L, platelet count≥75×10⁹/L, and hemoglobin≥9 g/dL (may have been transfused);
has adequate hepatic function defined by a total bilirubin level≤1.5× the upper limit of normal (ULN), an aspartate aminotransferase (AST) level≤2.5×ULN, and an alanine aminotransferase (ALT) level≤2.5×ULN, or, for subjects with documented metastatic disease to the liver, AST and ALT levels≤5×ULN, or has Gilbert Disease with a serum bilirubin level≤3×ULN; and/or
has adequate renal function defined by an estimated creatinine clearance>50 mL/min according to the Cockcroft-Gault formula.

In some embodiments, provided herein are methods of treating cancer in a subject, where the subject:
is male or female aged≥18 years;
has histologically or cytologically proven locally advanced or metastatic solid tumor from the following list, for which no standard therapy exists or standard therapy has failed;
   CRC
   NSCLC
   Esophageal adenocarcinoma
   HNSCC
   Gastric cancer
   Triple-negative breast cancer
   Renal cell carcinoma
   Pancreatic cancer
has adequate hematological function defined by WBC count≥3×10⁹/L, with ANC≥1.5×10⁹/L, lymphocyte count≥0.5×10⁹/L, platelet count≥75×10⁹/L, and hemoglobin≥9 g/dL (may have been transfused);
has adequate hepatic function defined by a total bilirubin level≤1.5× the ULN, an AST level≤2.5×ULN, and an ALT level≤2.5×ULN, or, for subjects with documented metastatic disease to the liver, AST and ALT levels≤5×ULN. Subjects with known Gilbert Disease who have serum bilirubin level≤3×ULN may be enrolled; and/or
has adequate renal function defined by an estimated creatinine clearance>50 mL/min according to the Cockcroft-Gault formula.

In some embodiments, provided herein are methods of treating cancer in a subject having HNSCC, where the subject:
is male or female aged≥18 years;
has histologically or cytologically documented relapsed or metastatic HNSCC. Primary tumor locations include oropharynx, oral cavity, hypopharynx, or larynx;
has radiographic disease progression while on or after having received either:
   pembrolizumab+platinum/5FU
   pembrolizumab monotherapy
   platinum/5FU+cetuximab
has received only 1 line of systemic therapy for the treatment of relapsed/metastatic disease;
has adequate hematological function defined by WBC count≥3×10⁹/L with ANC≥1.5×10⁹/L, lymphocyte count≥0.5×10⁹/L, platelet count≥75×10⁹/L, and hemoglobin≥9 g/dL (may have been transfused);
has adequate hepatic function defined by a total bilirubin level≤1.5× the ULN, an AST level≤2.5×ULN, and an ALT level≤2.5×ULN, or, for subjects with documented metastatic disease to the liver, AST and ALT levels≤5×ULN. Subjects with known Gilbert Disease who have a serum bilirubin level≤3×ULN may be enrolled; and/or
has adequate renal function defined by an estimated creatinine clearance>50 mL/min according to the Cockcroft-Gault formula or >30 mL/min.

In some embodiments, provided herein are methods of treating cancer in a subject having CRC, where the subject:
is male or female aged≥18 years;
has cytologically documented relapsed or metastatic colorectal cancer;
has received 1 line of either FOLFOX, CAPOX, FOLFIRI, or FOLFOXIRI with or without a biological agent and, if KRAS wild-type, has received prior treatment with cetuximab;
is not mismatch repair (MMR)/microsatellite instability (MSI) high;
has not received an anti-PD-1 or anti-PD-L1;
has radiographic disease progression while or after receiving treatment for their advanced (recurrent/unresectable/metastatic) disease;
has adequate hematological function defined by WBC count≥3×10⁹/L with ANC≥1.5×10⁹/L, lymphocyte count≥0.5×10⁹/L, platelet count≥75×10⁹/L, and hemoglobin≥9 g/dL (may have been transfused);
has adequate hepatic function defined by a total bilirubin level≤1.5× the ULN, an AST level≤2.5×ULN, and an ALT level≤2.5×ULN, or, for subjects with documented metastatic disease to the liver, AST and ALT levels≤5×ULN. Subjects with known Gilbert Disease who have a serum bilirubin level≤3×ULN may be enrolled; and/or
has adequate renal function defined by an estimated creatinine clearance>50 mL/min according to the Cockcroft-Gault formula or >30 mL/min.

In some embodiments, provided herein are methods of treating cancer in a subject having NSCLC, where the subject:
is male or female aged≥18 years;
has histologically-confirmed NSCLC meeting stage criteria for stage IIIB, stage IV, or recurrent disease, but does not have squamous histology;
has recurrent or progressive disease during or after platinum doublet-based chemotherapy OR has recurrent or progressive disease within 6 months after completing platinum-based chemotherapy for local disease;
has received and progressed on or after anti-PD-1/PD-L1 therapy;
has known status for actionable mutations (EGFR, ALK, ROS1, RET, etc.) when testing is available as per country/region standard of care practices; subjects with actionable mutations must have received and progressed on, have been intolerant to, or not be a candidate for standard tyrosine kinase inhibitors (TKIs) (as available per country/region standard of care practices);
has adequate hematological function defined by WBC count≥3×10⁹/L with ANC≥1.5×10⁹/L, lymphocyte count≥0.5×10⁹/L, platelet count≥75×10⁹/L, and hemoglobin≥9 g/dL (may have been transfused);
has adequate hepatic function defined by a total bilirubin level≤1.5× the ULN, an AST level≤2.5×ULN, and an ALT level≤2.5×ULN, or, for subjects with documented metastatic disease to the liver, AST and ALT levels≤5×ULN. Subjects with known Gilbert Disease who have a serum bilirubin level≤3×ULN may be enrolled; and/or
has adequate renal function defined by an estimated creatinine clearance>50 mL/min according to the Cockcroft-Gault formula or >30 mL/min.

In some embodiments, provided herein are methods of treating cancer in a subject with any of the formulations above in combination with an anti-PD-1 or anti-PD-L1, where the subject:
is eligible to receive nivolumab per its label for a malignancy of epithelial origin, has a disease for which no standard therapy exists, or standard therapy has failed for a malignancy of epithelial origin;
has not experienced a Grade 3 or 4 drug-related toxicity during and attributed to treatment with an anti-PD-1 or anti-PD-L1 therapy; and/or has not experienced a Grade 2 drug-related toxicity attributed to the use of an anti-PD-1 or an anti-PD-L1 that impacted either the lungs or the nervous system.

In some embodiments, provided herein are methods of treating cancer in a subject, where the subject:
has not had chemotherapy, radiotherapy (other than palliative bone-directed radiotherapy), or major surgery, or received another investigational agent within 28 days before the start of treatment.
is not undergoing concurrent anticancer treatment (e.g., cytoreductive therapy, radiotherapy [except for palliative bone-directed radiotherapy], immune therapy, or cytokine therapy [except for erythropoietin]), major surgery (excluding prior diagnostic biopsy), concurrent systemic therapy with steroids or other immunosuppressive agents, or use of any investigational drug within 28 days before the start of treatment.
has not had previous malignant disease other than the target malignancy within the last 3 years, with the exception of basal or squamous cell carcinoma of the skin, low-grade prostate cancer (Gleason score of ≤6), or cervical carcinoma in situ;
does not have brain metastases, unless all of the following criteria are met:
   CNS lesions are asymptomatic and previously treated.
   Subject does not require ongoing steroid treatment daily for replacement for adrenal insufficiency (except oral steroids at a dose less than ≤10 mg of prednisone [or equivalent]).
   Imaging demonstrates stability of disease 28 days from last treatment for CNS metastases.
has not received any organ transplantation, including autologous or allogeneic stem-cell transplantation.
does not have significant acute or chronic infection with hepatitis B virus (HBV)] or hepatitis C virus (IICV) tested during the screening window, as well as historic positive test for human immunodeficiency virus (HIV).
does not have preexisting autoimmune disease (except for subjects with vitiligo) needing treatment with systemic immunosuppressive agents for more than 28 days within the last 3 years, or clinically relevant immunodeficiencies (e.g., dysgammaglobulinemia or congenital immunodeficiencies);
does not have a known medical history of keratitis, ulcerative keratitis, or corneal perforation;
does not have known severe hypersensitivity reactions to monoclonal antibodies (≥Grade 3 of the National Cancer Institute-Common Terminology Criteria for Adverse Events [NCI-CTCAE] v5.0), any history of anaphylaxis, or uncontrolled asthma (i.e., 3 or more features of partly controlled asthma);
does not have persisting toxicity related to prior therapy>Grade 1 NCI-CTCAE v5.0;
however, alopecia<Grade 2, endocrinopathies<Grade 2, and sensory neuropathy<Grade 2 is acceptable.
has not received an anti-PD-1 or an anti-PD-L1 as a previous line of therapy AND has experienced either:
   a Grade 3 or 4 drug-related toxicity during and attributed to treatment with the anti-PD-1 or anti-PD-L1; or
   a Grade 2 drug-related toxicity that impacted either the lungs or the nervous system, caused by the administration of the anti-PD-1 or anti-PD-L1;
is not (for female subjects) pregnant or lactating;
does not have known alcohol or drug abuse; and/or
does not have serious cardiac illness or medical conditions, including but not limited to:
   History of New York Heart Association class III or IV heart failure or systolic dysfunction (left ventricular ejection fraction [LVEF]<55%);
   High-risk uncontrolled arrhythmias (e.g., tachycardia with a heart rate>100/min at rest);
   Significant ventricular arrhythmia (ventricular tachycardia) or higher-grade atrioventricular (AV) block (e.g., AV-block, second-degree AV-block Type 2 [Mobitz 2], or third-degree AV-block);
   Angina pectoris requiring anti-anginal medication;
   Clinically significant valvular heart disease;
   Evidence of transmural infarction on electrocardiogram (ECG);
   Poorly controlled hypertension (defined as systolic>180 mm Hg or diastolic>100 mm Hg);
   Clinically relevant uncontrolled cardiac risk factors or clinically relevant pulmonary disease; and/or
   Severe dyspnea at rest due to complications of advanced malignancy or requiring supplementary oxygen therapy;

### IV. Premedication Therapy

In certain embodiments, a multi-specific binding protein, or pharmaceutical formulation thereof, is administered with a premedication. In some embodiments, the premedication is an antihistamine and an antipyretic. In some embodiments, the premedication is a corticosteroid. In some embodiments, the premedication is (i) an antihistamine and an antipyretic, and (ii) a corticosteroid.

An antihistamine can be used to avoid or mitigate an allergic response (e.g., anaphylaxis) to the multi-specific binding protein, or pharmaceutical formulation thereof. Accordingly, in certain embodiments, the method further includes administering to the subject a therapeutically effective amount of an antihistamine. Exemplary antihistamines are disclosed in U.S. Pat. No. 10,898,693. In certain embodiments, the antihistamine used in the method disclosed herein is selected from the group consisting of crivastine, azelastine, bilastine, brompheniramine, buclizine, bromodiphenhydramine, carbinoxamine, cetirizine, cyclizine, chlorpheniramine, chlorodiphenhydramine, clemastine, cromolyn, cyproheptadine, desloratadine, dexbrompheniramine, dexchlorpheniramine, dimenhydrinate, dimetindene, diphenhydramine, doxylamine, ebastine, embramine, fexofenadine, hydroxyzine, levocetirizine, loratadine, nedocromil, olopatadine, phenindamine, pheniramine, phenyltoloxamine, promethazine, pyrilamine, rupatadine, tripelennamine, triprolidine, and combinations thereof. In certain embodiments, the antihistamine is diphenhydramine. In certain embodiments, the therapeutically effective amount of diphenhydramine is in the range of 10-100 mg, 20-100 mg, 30-100 mg, 40-100 mg, 50-100 mg, 10-50 mg, 20-50 mg, 30-50 mg, or 40-50 mg. In certain embodiments, the effective amount of diphenhydramine is 10 mg, 20 mg, 30 mg, 40 mg, 50 mg, 60 mg, 70 mg, 80 mg, 90 mg, or 100 mg.

In certain embodiments, the antihistamine is administered parenterally. In certain embodiments, the antihistamine is administered intravenously. In certain embodiments, the antihistamine is administered orally.

The antihistamine can be administered prior to, simultaneously with, or subsequent to the administration of the multi-specific binding protein. In certain embodiments, the antihistamine is administered within 2 hours, within 1.5 hours, within 1 hour (60 minutes), within 45 minutes, within 30 minutes, within 15 minutes, or immediately prior to the administration of the multi-specific binding protein or pharmaceutical formulation thereof (e.g., prior to the beginning of the administration of the multi-specific binding protein or pharmaceutical formulation thereof).

Where the method of treatment disclosed herein includes multiple doses (e.g., five or more doses) of the multi-specific binding protein or pharmaceutical formulation thereof, in certain embodiments, the antihistamine is administered with the first dose, the first two doses, the first three doses, the first four doses, or the first five doses of the multi-specific binding protein or pharmaceutical formulation thereof. Where the method of treatment disclosed herein includes multiple doses of the multi-specific binding protein or pharmaceutical formulation thereof, in certain embodiments, the antihistamine is administered before each and every infusion of the multi-specific binding protein or pharmaceutical formulation thereof. In some embodiments, where the method includes multiple cycles of treatment, the antihistamine is administered before each administration of the multi-specific binding protein or pharmaceutical formulation thereof.

An antipyretic can be used to prevent or reduce fever as a result of the administration of the multi-specific binding protein or pharmaceutical formulation thereof. Accordingly, in certain embodiments, the method further includes administering to the subject a therapeutically effective amount of an antipyretic. Exemplary antipyretics are disclosed in U.S. Patent Application Publication No. 2015/0342989. In certain embodiments, the antipyretic used in the method disclosed herein is selected from the group consisting of acetaminophen, salicylamide, salicyl salicylate, methyl salicylate, magnesium salicylate, faislamine, ethenzamide, diflunisal, choline magnesium salicylate, benorylate/benorilatem and amoxiprin, acetylsalicylate, ceclofenac, acemetacin, alclofenac, bromfenac, diclofenac, etodolac, indomethacin, nabumetone, oxametacin, proglumetacin, sulindac, tolmetin, iminoprofen, benoxaprofen, carprofen, dexibuprofen, dexketoprofen, fenbufen, fenoprofen, flunoxaprofen, flurbiprofen, ibuprofen, ibuproxam, indoprofen, ketoprofen, ketorolac, loxoprofen, naproxen, oxaprozin, pirprofen, suprofen, tiaprofenic acid, mefenamic acid, flufenamic acid, meclofenamic acid, tolfenamic acid, droxicam, lornoxicam, meloxicam, piroxicam, and tenoxicam, mpyrone, azapropazone, clofezone, kebuzone, metamizole, mofebutazone, oxyphenbutazone, phenazone, phenylbutazone, sulfinpyrazone, decoxib, rofecoxib, parecoxib, etoricoxib. In certain embodiments, the antipyretic is acetaminophen. In certain embodiments, the therapeutically effective amount of acetaminophen is in the range of 325-1000 mg, 400-1000 mg, 500-1000 mg, 600-1000 mg, 700-1000 mg, 800-1000 mg, 900-1000 mg, 325-800 mg, 400-800 mg, 500-800 mg, 600-800 mg, 700-800 mg, 325-600 mg, 400-600 mg, or 500-600 mg. In certain embodiments, the effective amount of acetaminophen is 325 mg, 500 mg, 650 mg, 700 mg, 800 mg, 900 mg, or 1000 mg.

In certain embodiments, the antipyretic is administered parenterally. In certain embodiments, the antipyretic is administered intravenously. In certain embodiments, the antipyretic is administered orally.

The antipyretic can be administered prior to, simultaneously with, or subsequent to the administration of the multi-specific binding protein or pharmaceutical formulation thereof. In certain embodiments, the antipyretic is administered within 2 hours, 1.5 hours, 1 hour (60 minutes), 45 minutes, 30 minutes, or 15 minutes prior to the administration of the multi-specific binding protein or pharmaceutical formulation thereof (e.g., prior to the beginning of the administration of the multi-specific binding protein or pharmaceutical formulation thereof). In certain embodiments, the antipyretic is administered simultaneously with the administration of the multi-specific binding protein or pharmaceutical formulation thereof. In certain embodiments, the antipyretic and the multi-specific binding protein are diluted into a single pharmaceutical composition administered to the subject. In certain embodiments, the duration of administration of the antipyretic and the duration of administration of the multi-specific binding protein completely or partially overlap. In certain embodiments, the antipyretic is administered within 2 hours, 1 hour, or 30 minutes subsequent to the administration of the multi-specific binding protein or pharmaceutical formulation thereof (e.g., subsequent to the beginning of the administration of the multi-specific binding protein or pharmaceutical formulation thereof). Where the method of treatment disclosed herein includes multiple doses of the multi-specific binding protein or pharmaceutical formulation thereof, in certain embodiments, the antipyretic is administered before each and every infusion of the multi-specific binding protein or pharmaceutical formulation thereof. In some embodiments, where the method includes multiple cycles of treatment, the antipyretic is administered before each administration of the multi-specific binding protein or pharmaceutical formulation thereof.

In some embodiments, the multi-specific binding protein or pharmaceutical formulations thereof of the present invention are administered with pre-medication treatment including an antihistamine and an antipyretic. For example, in some embodiments multi-specific binding protein or pharmaceutical formulations thereof of the present invention are administered with pre-medication treatment including acetaminophen and diphenhydramine. Where the method of treatment disclosed herein includes multiple doses of the multi-specific binding protein or pharmaceutical formulation thereof, in certain embodiments, the antihistamine and antipyretic are administered before each and every infusion of the multi-specific binding protein or pharmaceutical formulation thereof. In some embodiments, where the method includes multiple cycles of treatment, the anti-histamine and antipyretic are administered at each cycle before each infusion of the multi-specific binding protein or pharmaceutical formulation thereof.

The corticosteroids that are useful in the present invention generally include any steroid produced by the adrenocortex, including glucocorticoids and mineralocorticoids, and synthetic analogs and derivatives of naturally occurring corticosteroids having anti-inflammatory activity. In certain embodiments, the corticosteroid is a glucocorticoid. Glucocorticoids bind the glucocorticoid receptor and reduce inflammation by inhibiting the immune response. In certain embodiments, the corticosteroid is a mineralcorticoid. Mineral corticoids bind the mineralcorticoid receptor and act to regulate Na⁺/K⁺ concentrations in the serum. Some corticosteroids can have both glucocorticoid and mineralcorticoid functions. Examples of corticosteroids are disclosed in U.S. Pat. No. 10,799,599. In certain embodiments, the corticosteroid used in the method disclosed herein is selected from the group consisting of methylprednisolone, dexamethasone, hydrocortisone, prednisone, prednisolone, fluticasone, flumethasone, fluocinolone, budesonide, beclomethasone, ciclesonide, cortisone, triamcinolone, betamethasone, deflazacort, difluprednate, loteprednol, paramethasone, tixocortol, aldosterone, cloprednol, cortivazol, deoxycortone, desonide, desoximetasone, difluorocortolone, fluclorolone, fludrocortisone, flunisolide, fluocinonide, fluocortin butyl, fluorocortisone, fluorocortolone, fluorometholone, flurandrenolone, halcinonide, icomethasone, meprednisone, mometasone, rofleponide, RPR 106541, and their respective pharmaceutically acceptable derivatives, such as beclomethasone dipropionate (anhydrous or monohydrate), beclomethasone monopropionate, dexamethasone 21-isonicotinate, fluticasone propionate, icomethasone enbutate, tixocortol 21-pivalate, and triamcinolone acetonide, and pharmaceutically acceptable salts and/or derivatives thereof.

In certain embodiments, the glucocorticoid is methylprednisolone. Exemplary effective amounts of methylprednisolone can be in the range of 8-200 mg, 20-200 mg, 50-200 mg, 100-200 mg, 20-150 mg, 50-150 mg, or 100-150 mg. In certain embodiments, the effective amount of methylprednisolone by intravenous administration is 50 mg, 60 mg, 70 mg, 80 mg, 90 mg, 100 mg, 125 mg, or 150 mg. In certain embodiments, the effective amount of methylprednisolone by oral administration is 8 mg, 16 mg 32 mg, 48 mg, 64 mg, 80 mg, 96 mg, or 120 mg.

In certain embodiments, the glucocorticoid is dexamethasone. Exemplary effective amounts of dexamethasone can be in the range of 8-200 mg, 20-200 mg, 50-200 mg, 100-200 mg, 20-150 mg, 50-150 mg, 50-100 mg, or 100-150 mg. In certain embodiments, the effective amount of dexamethasone by intravenous administration is 20 mg, 30 mg, 40 mg, 50 mg, 60 mg, 70 mg, 80 mg, 90 mg, 100 mg, 125 mg, or 150 mg. In certain embodiments, the effective amount of dexamethasone by oral administration is 8 mg, 16 mg 32 mg, 48 mg, 64 mg, 80 mg, 96 mg, or 120 mg.

In certain embodiments, the corticosteroid is administered parenterally. In certain embodiments, the corticosteroid is administered intravenously. In certain embodiments, the corticosteroid is administered orally.

The corticosteroid can be administered prior to, simultaneously with, or subsequent to the administration of the multi-specific binding protein or pharmaceutical formulation thereof. In certain embodiments, the corticosteroid is administered within 6 hours, within 5 hours, within 4 hours, within 3 hours, within 2 hours, within 1 hour, within 30 minutes, within 15 minutes, or immediately prior to the administration of the multi-specific binding protein or pharmaceutical formulation thereof (e.g., prior to the beginning of the administration of the multi-specific binding protein or pharmaceutical formulation thereof). In certain embodiments, the corticosteroid is administered within 1 hour prior to the administration of the multi-specific binding protein (e.g., prior to the beginning of the administration of the multi-specific binding protein or pharmaceutical formulation thereof). In certain embodiments, the corticosteroid is administered simultaneously with the administration of the multi-specific binding protein or pharmaceutical formulation thereof. In certain embodiments, the corticosteroid and the multi-specific binding protein are diluted into a single pharmaceutical composition administered to the subject. In certain embodiments, the duration of administration of the corticosteroid and the duration of administration of the multi-specific binding protein, or pharmaceutical formulation thereof, completely or partially overlap. In certain embodiments, the corticosteroid is administered within 2 hours, 1 hour, or 30 minutes subsequent to the administration of the multi-specific binding protein or pharmaceutical formulation thereof (e.g., subsequent to the beginning of the administration of the multi-specific binding protein or pharmaceutical formulation thereof).

Where the method of treatment disclosed herein includes multiple doses of the multi-specific binding protein or pharmaceutical formulation thereof, in certain embodiments, the coritcosteroid is administered before the first infusion only.

In some embodiments, the multi-specific binding protein or pharmaceutical formulations thereof of the present invention are administered with pre-medication treatment including (i) an antihistamine and an antipyretic and (ii) a corticosteroid. For example, in some embodiments multi-specific binding protein or pharmaceutical formulations thereof of the present invention are administered with pre-medication treatment including (i) acetaminophen and diphenhydramine and (ii) methylprednisolone. Where the method of treatment disclosed herein includes multiple doses of the multi-specific binding protein or pharmaceutical formulation thereof, in certain embodiments, the antihistamine and antipyretic are administered before each and every infusion of the multi-specific binding protein or pharmaceutical formulation thereof, and the corticosteroid is administered before the first infusion only. In some embodiments, where the method includes multiple cycles of treatment, the antihistamine, antipyretic, and corticosteroid are administered at each and every cycle, and within each cycle, the antihistamine and antipyretic are administered before each and every infusion of the multi-specific binding protein or pharmaceutical formulation thereof, and the corticosteroid is administered before the first infusion of the multi-specific binding protein or pharmaceutical formulation thereof. In certain alternative embodiments, where the method includes multiple cycles of treatment, the antihistamine, antipyretic, and corticosteroid are administered at each and every cycle, and within each cycle, the antihistamine, antipyretic, and corticosteroid are administered before each and every infusion of the multi-specific binding protein or pharmaceutical formulation thereof.

Provided herein, in various embodiments, is a method of treating renal cell carcinoma in a subject in need thereof who has received an effective amount of pre-medication, where the method includes administering an effective amount of a multi-specific protein as disclosed herein, in various embodiments. The multi-specific binding protein can include: a first antigen binding site that binds NKG2D; a second antigen binding site that binds EGFR including (i) a VH having the CDR1, CDR2, and CDR3 sequences of SEQ ID NOs: 136, 157, and 138, respectively; and a VL having the CDR1, CDR2, and CDR3 sequences of SEQ ID NOs: 140, 141, and 151, respectively; (ii) a VH having the CDR1, CDR2, and CDR3 sequences of SEQ ID NOs: 136, 146, and 138, respectively; and a VL having the CDR1, CDR2, and CDR3 sequences of SEQ ID NOs: 140, 141, and 142, respectively; (iii) a VH having the CDR1, CDR2, and CDR3 sequences of SEQ ID NOs: 136, 146, and 138, respectively; and a VL having the CDR1, CDR2, and CDR3 sequences of SEQ ID NOs: 140, 141, and 151, respectively; (iv) a VH having the CDR1, CDR2, and CDR3 sequences of SEQ ID NOs: 136, 137, and 138, respectively; and a VL having the CDR1, CDR2, and CDR3 sequences of SEQ ID NOs: 140, 141, and 151, respectively; or (v) a VH having the CDR1, CDR2, and CDR3 sequences of SEQ ID NOs: 136, 137, and 138, respectively; and a VL having the CDR1, CDR2, and CDR3 sequences of SEQ ID NOs: 140, 141, and 142, respectively; and an antibody Fc domain or a portion thereof sufficient to bind CD16, or a third antigen-binding site that binds CD16. As provided herein, in various embodiments, the pre-medication includes: (i) an anti-histamine (e.g., diphenhydramine) and an anti-pyretic (e.g., acetaminophen); and (ii) a corticosteroid (e.g. methylprednisolone). In some embodiments, where the method of treatment includes administering multiple doses of the multi-specific binding protein disclosed herein, the antihistamine and antipyretic are administered before each and every infusion of the multi-specific binding protein or pharmaceutical formulation thereof, and the corticosteroid is administered before the first infusion only. In some embodiments, where the method includes multiple cycles of treatment, the antihistamine, antipyretic, and corticosteroid are administered at each and every cycle, and within each cycle, the antihistamine and antipyretic are administered before each and every infusion of the multi-specific binding protein or pharmaceutical formulation thereof, and the corticosteroid is administered before the first infusion of the multi-specific binding protein or pharmaceutical formulation thereof. In certain alternative embodiments, where the method includes multiple cycles of treatment, the antihistamine, antipyretic, and corticosteroid are administered at each and every cycle, and within each cycle, the antihistamine, antipyretic, and corticosteroid are administered before each and every infusion of the multi-specific binding protein or pharmaceutical formulation thereof.

Provided herein, in various embodiments, is a method of treating renal cell carcinoma in a subject in need thereof who has received an effective amount of pre-medication, where the method includes administering a formulation including an effective amount of a multi-specific protein as disclosed herein, in various embodiments. The formulation can include: (1) a multi-specific binding protein including a first antigen binding site that binds NKG2D, a second antigen binding site that binds EGFR including (i) a VH having the CDR1, CDR2, and CDR3 sequences of SEQ ID NOs: 136, 157, and 138, respectively; and a VL having the CDR1, CDR2, and CDR3 sequences of SEQ ID NOs: 140, 141, and 151, respectively, (ii) a VH having the CDR1, CDR2, and CDR3 sequences of SEQ ID NOs: 136, 146, and 138, respectively, and a VL having the CDR1, CDR2, and CDR3 sequences of SEQ ID NOs: 140, 141, and 142, respectively, (iii) a VH having the CDR1, CDR2, and CDR3 sequences of SEQ ID NOs: 136, 146, and 138, respectively; and a VL having the CDR1, CDR2, and CDR3 sequences of SEQ ID NOs: 140, 141, and 151, respectively, (iv) a VH having the CDR1, CDR2, and CDR3 sequences of SEQ ID NOs: 136, 137, and 138, respectively, and a VL having the CDR1, CDR2, and CDR3 sequences of SEQ ID NOs: 140, 141, and 151, respectively, or (v) a VH having the CDR1, CDR2, and CDR3 sequences of SEQ ID NOs: 136, 137, and 138, respectively, and a VL having the CDR1, CDR2, and CDR3 sequences of SEQ ID NOs: 140, 141, and 142, respectively, and an antibody Fc domain or a portion thereof sufficient to bind CD16, or a third antigen-binding site that binds CD16; and (2) one or more of (i) citrate, (ii) a sugar or sugar alcohol, and (iii) a polysorbate, at pH 6.0 to 7.0. As provided herein, in various embodiments, the pre-medication includes: (i) an anti-histamine (e.g., diphenhydramine) and an anti-pyretic (e.g., acetaminophen); and (ii) a corticosteroid (e.g. methylprednisolone). In some embodiments, where the method of treatment includes administering multiple doses of the formulation including a multi-specific binding protein disclosed herein, the antihistamine and antipyretic are administered before each and every infusion of the multi-specific binding protein or pharmaceutical formulation thereof, and the corticosteroid is administered before the first infusion only. In some embodiments, where the method includes multiple cycles of treatment, the antihistamine, antipyretic, and corticosteroid are administered before each and every infusion of the multi-specific binding protein or pharmaceutical formulation thereof.

Provided herein, in various embodiments, is a method of treating gastric cancer in a subject in need thereof who has received an effective amount of pre-medication, where the method includes administering a formulation including an effective amount of a multi-specific protein as disclosed herein, in various embodiments. The formulation can include: (1) a multi-specific binding protein including a first antigen binding site that binds NKG2D, a second antigen binding site that binds EGFR including (i) a VH having the CDR1, CDR2, and CDR3 sequences of SEQ ID NOs: 136, 157, and 138, respectively; and a VL having the CDR1, CDR2, and CDR3 sequences of SEQ ID NOs: 140, 141, and 151, respectively, (ii) a VH having the CDR1, CDR2, and CDR3 sequences of SEQ ID NOs: 136, 146, and 138, respectively, and a VL having the CDR1, CDR2, and CDR3 sequences of SEQ ID NOs: 140, 141, and 142, respectively, (iii) a VH having the CDR1, CDR2, and CDR3 sequences of SEQ ID NOs: 136, 146, and 138, respectively; and a VL having the CDR1, CDR2, and CDR3 sequences of SEQ ID NOs: 140, 141, and 151, respectively, (iv) a VH having the CDR1, CDR2, and CDR3 sequences of SEQ ID NOs: 136, 137, and 138, respectively, and a VL having the CDR1, CDR2, and CDR3 sequences of SEQ ID NOs: 140, 141, and 151, respectively, or (v) a VH having the CDR1, CDR2, and CDR3 sequences of SEQ ID NOs: 136, 137, and 138, respectively, and a VL having the CDR1, CDR2, and CDR3 sequences of SEQ ID NOs: 140, 141, and 142, respectively, and an antibody Fc domain or a portion thereof sufficient to bind CD16, or a third antigen-binding site that binds CD16; and (2) one or more of (i) citrate, (ii) a sugar or sugar alcohol, and (iii) a polysorbate, at pH 6.0 to 7.0. As provided herein, in various embodiments, the pre-medication includes: (i) an anti-histamine (e.g., diphenhydramine) and an anti-pyretic (e.g., acetaminophen); and (ii) a corticosteroid (e.g. methylprednisolone). In some embodiments, where the method of treatment includes administering multiple doses of the formulation including a multi-specific binding protein disclosed herein, the antihistamine and antipyretic are administered before each and every infusion of the multi-specific binding protein or pharmaceutical formulation thereof, and the corticosteroid is administered before the first infusion only. In some embodiments, where the method includes multiple cycles of treatment, the antihistamine, antipyretic, and corticosteroid are administered at each and every cycle, and within each cycle, the antihistamine and antipyretic are administered before each and every infusion of the multi-specific binding protein or pharmaceutical formulation thereof, and the corticosteroid is administered before the first infusion of the multi-specific binding protein or pharmaceutical formulation thereof. In certain alternative embodiments, where the method includes multiple cycles of treatment, the antihistamine, antipyretic, and corticosteroid are administered at each and every cycle, and within each cycle, the antihistamine, antipyretic, and corticosteroid are administered before each and every infusion of the multi-specific binding protein or pharmaceutical formulation thereof.

Provided herein, in various embodiments, is a method of treating pancreatic cancer in a subject in need thereof who has received an effective amount of pre-medication, where the method includes administering a formulation including an effective amount of a multi-specific protein as disclosed herein, in various embodiments. The formulation can include: (1) a multi-specific binding protein including a first antigen binding site that binds NKG2D, a second antigen binding site that binds EGFR including (i) a VH having the CDR1, CDR2, and CDR3 sequences of SEQ ID NOs: 136, 157, and 138, respectively; and a VL having the CDR1, CDR2, and CDR3 sequences of SEQ ID NOs: 140, 141, and 151, respectively, (ii) a VH having the CDR1, CDR2, and CDR3 sequences of SEQ ID NOs: 136, 146, and 138, respectively, and a VL having the CDR1, CDR2, and CDR3 sequences of SEQ ID NOs: 140, 141, and 142, respectively, (iii) a VH having the CDR1, CDR2, and CDR3 sequences of SEQ ID NOs: 136, 146, and 138, respectively; and a VL having the CDR1, CDR2, and CDR3 sequences of SEQ ID NOs: 140, 141, and 151, respectively, (iv) a VH having the CDR1, CDR2, and CDR3 sequences of SEQ ID NOs: 136, 137, and 138, respectively, and a VL having the CDR1, CDR2, and CDR3 sequences of SEQ ID NOs: 140, 141, and 151, respectively, or (v) a VH having the CDR1, CDR2, and CDR3 sequences of SEQ ID NOs: 136, 137, and 138, respectively, and a VL having the CDR1, CDR2, and CDR3 sequences of SEQ ID NOs: 140, 141, and 142, respectively, and an antibody Fc domain or a portion thereof sufficient to bind CD16, or a third antigen-binding site that binds CD16; and (2) one or more of (i) citrate, (ii) a sugar or sugar alcohol, and (iii) a polysorbate, at pH 6.0 to 7.0. As provided herein, in various embodiments, the pre-medication includes: (i) an anti-histamine (e.g., diphenhydramine) and an anti-pyretic (e.g., acetaminophen); and (ii) a corticosteroid (e.g. methylprednisolone). In some embodiments, where the method of treatment includes administering multiple doses of the formulation including a multi-specific binding protein disclosed herein, the antihistamine and antipyretic are administered before each and every infusion of the multi-specific binding protein or pharmaceutical formulation thereof, and the corticosteroid is administered before the first infusion only. In some embodiments, where the method includes multiple cycles of treatment, the antihistamine, antipyretic, and corticosteroid are administered at each and every cycle, and within each cycle, the antihistamine and antipyretic are administered before each and every infusion of the multi-specific binding protein or pharmaceutical formulation thereof, and the corticosteroid is administered before the first infusion of the multi-specific binding protein or pharmaceutical formulation thereof. In certain alternative embodiments, where the method includes multiple cycles of treatment, the antihistamine, antipyretic, and corticosteroid are administered at each and every cycle, and within each cycle, the antihistamine, antipyretic, and corticosteroid are administered before each and every infusion of the multi-specific binding protein or pharmaceutical formulation thereof.

### V. Combination Therapy

Another aspect of the present application provides for combination therapy. A multi-specific binding protein, or pharmaceutical formulation thereof, described herein can be used in combination with additional therapeutic agents to treat cancer.

In some embodiments, the multi-specific binding protein, or pharmaceutical formulation thereof, as described herein is administered in combination with an anti-PD-1 or an anti-PD-L1 therapy. Non-limiting examples of anti-PD-1 therapies include pembrolizumab, nivolumab, cemiplimab, dostarlimab, JTZ-4014, spartalizumab, camrelizumab, sintilimab, tislelizumab, toripalimab, INCMGA00012, AMP-224, and AMP-514. Non-limiting examples of anti-PD-L1 therapies include atezolizumab, avelumab, durvalumab, KNO35m CK-301, AUNP12, CA-170, or BMS-986189.

In some embodiments, the anti-PD-1 or anti-PD-L1 therapy is selected from nivolumab, pembrolizumab, durvalumab, or atezolizumab. In some embodiments, an effective amount of anti-PD-1 or anti-PD-L1 therapy can be in the range of 1-1000 mg, 100-1000 mg, 200-1000 mg, 300-1000 mg, 400-1000 mg, 500-1000 mg, 600-1000 mg, 700-1000 mg, 800-1000 mg, 900-1000 mg, 1-800 mg, 100-800 mg, 200-800 mg, 300-800 mg, 400-800 mg, 500-800 mg, 600-800 mg, 700-800 mg, 1-600 mg, 100-600 mg, 200-600 mg, 300-600 mg, 400-600 mg, 500-600 mg, 1-400 mg, 100-400 mg, 200-400 mg, 300-400 mg, 1-200 mg, or 100-200 mg. In some embodiments, an effective amount of anti-PD-1 or anti-PD-L1 therapy is about 400 mg, about 420 mg, about 440 mg, about 460 mg, about 480 mg, about 500 mg, about 520 mg, or about 540 mg.

In some embodiments, the anti-PD-1 or anti-PD-L1 therapy is administered parenterally. In some embodiments, the anti-PD-1 or anti-PD-L1 therapy is administered intravenously. In some embodiments, the anti-PD-1 or anti-PD-L1 therapy is administered orally.

In some embodiments, the anti-PD-1 or anti-PD-L1 therapy is administered in combination with a multi-specific binding protein, or formulation thereof, of the present invention for the treatment of an unresectable solid tumor. In some embodiments, the anti-PD-1 or anti-PD-L1 therapy is administered in combination with a multi-specific binding protein, or formulation thereof, of the present invention for the treatment of a recurrent solid tumor. In some embodiments, the anti-PD-1 or anti-PD-L1 therapy is administered in combination with a multi-specific binding protein, or formulation thereof, of the present invention for the treatment of an advanced solid tumor, for which there is no effective standard therapy. In some embodiments, the anti-PD-1 or anti-PD-L1 therapy is administered in combination with a multi-specific binding protein, or formulation thereof, of the present invention for the treatment of a cancer in a subject that is intolerant of standard therapies. In some embodiments, the anti-PD-1 or anti-PD-L1 therapy is administered in combination with a multi-specific binding protein, or formulation thereof that is administered once weekly in 4-week treatment cycles. In some embodiments, the anti-PD-1 or anti-PD-L1 therapy is administered in combination with a multi-specific binding protein, or formulation thereof, of the present invention for the treatment of a cancer in a subject for which no standard therapy exists or, even when a standard therapy exists, the standard therapy of the subject has failed for a malignancy of epithelial origin. In some embodiments, the anti-PD-1 or anti-PD-L1 therapy is administered in combination with a multi-specific binding protein, or formulation thereof, of the present invention for the treatment of cancer in a subject characterized as having previously received an anti-PD-1 or anti-PD-L1 therapy.

In some embodiments, the anti-PD-1 or anti-PD-L1 therapy is administered in combination with a multi-specific binding protein, or formulation thereof, of the present invention for the treatment of head and neck squamous cell carcinoma (HNSCC) in a subject. In some embodiments, the anti-PD-1 or anti-PD-L1 therapy is administered in combination with a multi-specific binding protein, or formulation thereof, of the present invention for the treatment of HNSCC in a subject characterized as having radiographic disease progression while on or after having received: (i) pembrolizumab and platinum/5FU, (ii) pembrolizumab monotherapy, or (iii) platinum/5FU and cetuximab.

In some embodiments, the anti-PD-1 or anti-PD-L1 therapy is administered in combination with a multi-specific binding protein, or formulation thereof, of the present invention for the treatment of relapsed or metastatic colorectal cancer (CRC) in a subject. In some embodiments, the anti-PD-1 or anti-PD-L1 therapy is administered in combination with a multi-specific binding protein, or formulation thereof, of the present invention for the treatment of CRC in a subject, who has been treated with FOLFOX, CAPOX, FOLFIRI, or FOLFOXIRI, with or without a biological agent. In some embodiments, the anti-PD-1 or anti-PD-L1 therapy is administered in combination with a multi-specific binding protein, or formulation thereof, of the present invention for the treatment of CRC in a subject characterized as not having high mismatch repair/microsatellite instability. In some embodiments, the anti-PD-1 or anti-PD-L1 therapy is administered in combination with a multi-specific binding protein, or formulation thereof, of the present invention for the treatment of CRC in a subject characterized as not previously treated with an anti-PD-1 or an anti-PD-L1 therapy. In some embodiments, the anti-PD-1 or anti-PD-L1 therapy is administered in combination with a multi-specific binding protein, or formulation thereof, of the present invention for the treatment of CRC in a subject characterized as having radiographic disease progression while or after receiving treatment for advanced (recurrent/unresectable/metastatic) cancer.

In some embodiments, the anti-PD-1 or anti-PD-L1 therapy is administered in combination with a multi-specific binding protein, or formulation thereof, of the present invention for the treatment of non-small-cell lung cancer (NSCLC) in a subject characterized as having recurrent or progressive disease during or after platinum doublet-based chemotherapy, or having recurrent or progressive disease within 6 months after completing platinum-based chemotherapy for local disease. In some embodiments, the anti-PD-1 or anti-PD-L1 therapy is administered in combination with a multi-specific binding protein, or formulation thereof, of the present invention for the treatment of NSCLC in a subject, who has previously received an anti-PD-1 or anti-PD-L1 therapy.

In some embodiments, the anti-PD-1 or anti-PD-L1 therapy is administered in combination with a multi-specific binding protein, or formulation thereof, of the present invention for the treatment of esophageal adenocarcinoma in a subject. In some embodiments, the anti-PD-1 or anti-PD-L1 therapy is administered in combination with a multi-specific binding protein, or formulation thereof, of the present invention for the treatment of triple-negative breast cancer in a subject. In some embodiments, the anti-PD-1 or anti-PD-L1 therapy is administered in combination with a multi-specific binding protein, or formulation thereof, of the present invention for the treatment of renal cell carcinoma in a subject. In some embodiments, the anti-PD-1 or anti-PD-L1 therapy is administered in combination with a multi-specific binding protein, or formulation thereof, of the present invention for the treatment of gastric cancer in a subject. In some embodiments, the anti-PD-1 or anti-PD-L1 therapy is administered in combination with a multi-specific binding protein, or formulation thereof, of the present invention for the treatment of pancreatic cancer in a subject.

In some embodiments, the anti-PD-1 or anti-PD-L1 therapy is administered in combination with a multi-specific binding protein, or formulation thereof, of the present invention in a subject who has received pre-medication therapy. In some embodiments, the pre-medication therapy includes (i) an antihistamine and an antipyretic and/or (ii) a corticosteroid. Where the method of treatment includes multiple doses of the multi-specific binding protein or pharmaceutical formulation thereof, in certain embodiments, the antihistamine and antipyretic are administered before each and every infusion of the multi-specific binding protein or pharmaceutical formulation thereof, and/or the corticosteroid is administered before the first infusion only. In some embodiments, where the method includes multiple cycles of treatment, the antihistamine, antipyretic, and corticosteroid are administered at each and every cycle, and within each cycle, the antihistamine and antipyretic are administered before each and every infusion of the multi-specific binding protein or pharmaceutical formulation thereof, and the corticosteroid is administered before the first infusion of the multi-specific binding protein or pharmaceutical formulation thereof. In certain alternative embodiments, where the method includes multiple cycles of treatment, the antihistamine, antipyretic, and corticosteroid are administered at each and every cycle, and within each cycle, the antihistamine, antipyretic, and corticosteroid are administered before each and every infusion of the multi-specific binding protein or pharmaceutical formulation thereof.

Provided herein, in various embodiments, is a method of treating renal cell carcinoma in a subject in need thereof including administering an effective amount of a multi-specific protein as disclosed herein, in various embodiments, in combination with an anti-PD-1 or an anti-PD-L1 therapy. The multi-specific binding protein can include: a first antigen binding site that binds NKG2D; a second antigen binding site that binds EGFR including (i) a VH having the CDR1, CDR2, and CDR3 sequences of SEQ ID NOs: 136, 157, and 138, respectively; and a VL having the CDR1, CDR2, and CDR3 sequences of SEQ ID NOs: 140, 141, and 151, respectively; (ii) a VH having the CDR1, CDR2, and CDR3 sequences of SEQ ID NOs: 136, 146, and 138, respectively; and a VL having the CDR1, CDR2, and CDR3 sequences of SEQ ID NOs: 140, 141, and 142, respectively; (iii) a VH having the CDR1, CDR2, and CDR3 sequences of SEQ ID NOs: 136, 146, and 138, respectively; and a VL having the CDR1, CDR2, and CDR3 sequences of SEQ ID NOs: 140, 141, and 151, respectively; (iv) a VH having the CDR1, CDR2, and CDR3 sequences of SEQ ID NOs: 136, 137, and 138, respectively; and a VL having the CDR1, CDR2, and CDR3 sequences of SEQ ID NOs: 140, 141, and 151, respectively; or (v) a VH having the CDR1, CDR2, and CDR3 sequences of SEQ ID NOs: 136, 137, and 138, respectively; and a VL having the CDR1, CDR2, and CDR3 sequences of SEQ ID NOs: 140, 141, and 142, respectively; and an antibody Fc domain or a portion thereof sufficient to bind CD16, or a third antigen-binding site that binds CD16. As provided herein, in various embodiments, the anti-PD-1 or the anti-PD-L1 therapy includes nivolumab.

Also provided herein, in various embodiments, is a method of treating renal cell carcinoma in a subject in need thereof who has received an effective amount of pre-medication, where the method includes administering an effective amount of a multi-specific protein as disclosed herein, in various embodiments, in combination with an anti-PD-1 or an anti-PD-L1 therapy. The multi-specific binding protein can include: a first antigen binding site that binds NKG2D; a second antigen binding site that binds EGFR including (i) a VH having the CDR1, CDR2, and CDR3 sequences of SEQ ID NOs: 136, 157, and 138, respectively; and a VL having the CDR1, CDR2, and CDR3 sequences of SEQ ID NOs: 140, 141, and 151, respectively; (ii) a VH having the CDR1, CDR2, and CDR3 sequences of SEQ ID NOs: 136, 146, and 138, respectively; and a VL having the CDR1, CDR2, and CDR3 sequences of SEQ ID NOs: 140, 141, and 142, respectively; (iii) a VH having the CDR1, CDR2, and CDR3 sequences of SEQ ID NOs: 136, 146, and 138, respectively; and a VL having the CDR1, CDR2, and CDR3 sequences of SEQ ID NOs: 140, 141, and 151, respectively; (iv) a VH having the CDR1, CDR2, and CDR3 sequences of SEQ ID NOs: 136, 137, and 138, respectively; and a VL having the CDR1, CDR2, and CDR3 sequences of SEQ ID NOs: 140, 141, and 151, respectively; or (v) a VH having the CDR1, CDR2, and CDR3 sequences of SEQ ID NOs: 136, 137, and 138, respectively; and a VL having the CDR1, CDR2, and CDR3 sequences of SEQ ID NOs: 140, 141, and 142, respectively; and an antibody Fc domain or a portion thereof sufficient to bind CD16, or a third antigen-binding site that binds CD16. As provided herein, in various embodiments, the anti-PD-1 or the anti-PD-L1 therapy includes nivolumab, and the pre-medication includes an (i) an anti-histamine (e.g., diphenhydramine) and an anti-pyretic (e.g., acetaminophen); and (ii) a corticosteroid (e.g. methylprednisolone). In some embodiments, where the method of treatment includes administering multiple doses of the formulation including a multi-specific binding protein disclosed herein, the antihistamine and antipyretic are administered before each and every infusion of the multi-specific binding protein or pharmaceutical formulation thereof, and the corticosteroid is administered before the first infusion only. In some embodiments, where the method includes multiple cycles of treatment, the antihistamine, antipyretic, and corticosteroid are administered at each and every cycle, and within each cycle, the antihistamine and antipyretic are administered before each and every infusion of the multi-specific binding protein or pharmaceutical formulation thereof, and the corticosteroid is administered before the first infusion of the multi-specific binding protein or pharmaceutical formulation thereof. In certain alternative embodiments, where the method includes multiple cycles of treatment, the antihistamine, antipyretic, and corticosteroid are administered at each and every cycle, and within each cycle, the antihistamine, antipyretic, and corticosteroid are administered before each and every infusion of the multi-specific binding protein or pharmaceutical formulation thereof.

Provided herein, in various embodiments, is a method of treating renal cell carcinoma in a subject in need thereof including administering a formulation including an effective amount of a multi-specific protein as disclosed herein, in various embodiments, in combination with an anti-PD-1 or an anti-PD-L1 therapy. The formulation can include: (1) a multi-specific binding protein including a first antigen binding site that binds NKG2D, a second antigen binding site that binds EGFR including (i) a VH having the CDR1, CDR2, and CDR3 sequences of SEQ ID NOs: 136, 157, and 138, respectively; and a VL having the CDR1, CDR2, and CDR3 sequences of SEQ ID NOs: 140, 141, and 151, respectively, (ii) a VH having the CDR1, CDR2, and CDR3 sequences of SEQ ID NOs: 136, 146, and 138, respectively, and a VL having the CDR1, CDR2, and CDR3 sequences of SEQ ID NOs: 140, 141, and 142, respectively, (iii) a VH having the CDR1, CDR2, and CDR3 sequences of SEQ ID NOs: 136, 146, and 138, respectively; and a VL having the CDR1, CDR2, and CDR3 sequences of SEQ ID NOs: 140, 141, and 151, respectively, (iv) a VH having the CDR1, CDR2, and CDR3 sequences of SEQ ID NOs: 136, 137, and 138, respectively, and a VL having the CDR1, CDR2, and CDR3 sequences of SEQ ID NOs: 140, 141, and 151, respectively, or (v) a VH having the CDR1, CDR2, and CDR3 sequences of SEQ ID NOs: 136, 137, and 138, respectively, and a VL having the CDR1, CDR2, and CDR3 sequences of SEQ ID NOs: 140, 141, and 142, respectively, and an antibody Fc domain or a portion thereof sufficient to bind CD16, or a third antigen-binding site that binds CD16; and (2) one or more of (i) citrate, (ii) a sugar or sugar alcohol, and (iii) a polysorbate, at pH 6.0 to 7.0. As provided herein, in various embodiments, the anti-PD-1 or the anti-PD-L1 therapy includes nivolumab.

Also provided herein, in various embodiments, is a method of treating renal cell carcinoma in a subject in need thereof who has received an effective amount of pre-medication, where the method includes administering a formulation including an effective amount of a multi-specific protein as disclosed herein, in various embodiments, in combination with an anti-PD-1 or an anti-PD-L1 therapy. The formulation can include: (1) a multi-specific binding protein including a first antigen binding site that binds NKG2D, a second antigen binding site that binds EGFR including (i) a VH having the CDR1, CDR2, and CDR3 sequences of SEQ ID NOs: 136, 157, and 138, respectively; and a VL having the CDR1, CDR2, and CDR3 sequences of SEQ ID NOs: 140, 141, and 151, respectively, (ii) a VH having the CDR1, CDR2, and CDR3 sequences of SEQ ID NOs: 136, 146, and 138, respectively, and a VL having the CDR1, CDR2, and CDR3 sequences of SEQ ID NOs: 140, 141, and 142, respectively, (iii) a VH having the CDR1, CDR2, and CDR3 sequences of SEQ ID NOs: 136, 146, and 138, respectively; and a VL having the CDR1, CDR2, and CDR3 sequences of SEQ ID NOs: 140, 141, and 151, respectively, (iv) a VH having the CDR1, CDR2, and CDR3 sequences of SEQ ID NOs: 136, 137, and 138, respectively, and a VL having the CDR1, CDR2, and CDR3 sequences of SEQ ID NOs: 140, 141, and 151, respectively, or (v) a VH having the CDR1, CDR2, and CDR3 sequences of SEQ ID NOs: 136, 137, and 138, respectively, and a VL having the CDR1, CDR2, and CDR3 sequences of SEQ ID NOs: 140, 141, and 142, respectively, and an antibody Fc domain or a portion thereof sufficient to bind CD16, or a third antigen-binding site that binds CD16; and (2) one or more of (i) citrate, (ii) a sugar or sugar alcohol, and (iii) a polysorbate, at pH 6.0 to 7.0. As provided herein, in various embodiments, the anti-PD-1 or the anti-PD-L1 therapy includes nivolumab, and the pre-medication includes an (i) an anti-histamine (e.g., diphenhydramine) and an anti-pyretic (e.g., acetaminophen); and (ii) a corticosteroid (e.g. methylprednisolone). In some embodiments, where the method of treatment includes administering multiple doses of the formulation including a multi-specific binding protein disclosed herein, the antihistamine and antipyretic are administered before each and every infusion of the multi-specific binding protein or pharmaceutical formulation thereof, and the corticosteroid is administered before the first infusion only. In some embodiments, where the method includes multiple cycles of treatment, the antihistamine, antipyretic, and corticosteroid are administered at each and every cycle, and within each cycle, the antihistamine and antipyretic are administered before each and every infusion of the multi-specific binding protein or pharmaceutical formulation thereof, and the corticosteroid is administered before the first infusion of the multi-specific binding protein or pharmaceutical formulation thereof. In certain alternative embodiments, where the method includes multiple cycles of treatment, the antihistamine, antipyretic, and corticosteroid are administered at each and every cycle, and within each cycle, the antihistamine, antipyretic, and corticosteroid are administered before each and every infusion of the multi-specific binding protein or pharmaceutical formulation thereof.

Provided herein, in various embodiments, is a method of treating gastric cancer in a subject in need thereof including administering a formulation including an effective amount of a multi-specific protein as disclosed herein, in various embodiments, in combination with an anti-PD-1 or an anti-PD-L1 therapy. The formulation can include: (1) a multi-specific binding protein including a first antigen binding site that binds NKG2D, a second antigen binding site that binds EGFR including (i) a VH having the CDR1, CDR2, and CDR3 sequences of SEQ ID NOs: 136, 157, and 138, respectively; and a VL having the CDR1, CDR2, and CDR3 sequences of SEQ ID NOs: 140, 141, and 151, respectively, (ii) a VH having the CDR1, CDR2, and CDR3 sequences of SEQ ID NOs: 136, 146, and 138, respectively, and a VL having the CDR1, CDR2, and CDR3 sequences of SEQ ID NOs: 140, 141, and 142, respectively, (iii) a VH having the CDR1, CDR2, and CDR3 sequences of SEQ ID NOs: 136, 146, and 138, respectively; and a VL having the CDR1, CDR2, and CDR3 sequences of SEQ ID NOs: 140, 141, and 151, respectively, (iv) a VH having the CDR1, CDR2, and CDR3 sequences of SEQ ID NOs: 136, 137, and 138, respectively, and a VL having the CDR1, CDR2, and CDR3 sequences of SEQ ID NOs: 140, 141, and 151, respectively, or (v) a VH having the CDR1, CDR2, and CDR3 sequences of SEQ ID NOs: 136, 137, and 138, respectively, and a VL having the CDR1, CDR2, and CDR3 sequences of SEQ ID NOs: 140, 141, and 142, respectively, and an antibody Fc domain or a portion thereof sufficient to bind CD16, or a third antigen-binding site that binds CD16; and (2) one or more of (i) citrate, (ii) a sugar or sugar alcohol, and (iii) a polysorbate, at pH 6.0 to 7.0. As provided herein, in various embodiments, the anti-PD-1 or the anti-PD-L1 therapy includes nivolumab.

Also provided herein, in various embodiments, is a method of treating gastric cancer in a subject in need thereof who has received an effective amount of pre-medication, where the method includes administering a formulation including an effective amount of a multi-specific protein as disclosed herein, in various embodiments, in combination with an anti-PD-1 or an anti-PD-L1 therapy. The formulation can include: (1) a multi-specific binding protein including a first antigen binding site that binds NKG2D, a second antigen binding site that binds EGFR including (i) a VH having the CDR1, CDR2, and CDR3 sequences of SEQ ID NOs: 136, 157, and 138, respectively; and a VL having the CDR1, CDR2, and CDR3 sequences of SEQ ID NOs: 140, 141, and 151, respectively, (ii) a VH having the CDR1, CDR2, and CDR3 sequences of SEQ ID NOs: 136, 146, and 138, respectively, and a VL having the CDR1, CDR2, and CDR3 sequences of SEQ ID NOs: 140, 141, and 142, respectively, (iii) a VH having the CDR1, CDR2, and CDR3 sequences of SEQ ID NOs: 136, 146, and 138, respectively; and a VL having the CDR1, CDR2, and CDR3 sequences of SEQ ID NOs: 140, 141, and 151, respectively, (iv) a VH having the CDR1, CDR2, and CDR3 sequences of SEQ ID NOs: 136, 137, and 138, respectively, and a VL having the CDR1, CDR2, and CDR3 sequences of SEQ ID NOs: 140, 141, and 151, respectively, or (v) a VH having the CDR1, CDR2, and CDR3 sequences of SEQ ID NOs: 136, 137, and 138, respectively, and a VL having the CDR1, CDR2, and CDR3 sequences of SEQ ID NOs: 140, 141, and 142, respectively, and an antibody Fc domain or a portion thereof sufficient to bind CD16, or a third antigen-binding site that binds CD16; and (2) one or more of (i) citrate, (ii) a sugar or sugar alcohol, and (iii) a polysorbate, at pH 6.0 to 7.0. As provided herein, in various embodiments, the anti-PD-1 or the anti-PD-L1 therapy includes nivolumab, and the pre-medication includes an (i) an anti-histamine (e.g., diphenhydramine) and an anti-pyretic (e.g., acetaminophen); and (ii) a corticosteroid (e.g. methylprednisolone). In some embodiments, where the method of treatment includes administering multiple doses of the formulation including a multi-specific binding protein disclosed herein, the antihistamine and antipyretic are administered before each and every infusion of the multi-specific binding protein or pharmaceutical formulation thereof, and the corticosteroid is administered before the first infusion only. In some embodiments, where the method includes multiple cycles of treatment, the antihistamine, antipyretic, and corticosteroid are administered at each and every cycle, and within each cycle, the antihistamine and antipyretic are administered before each and every infusion of the multi-specific binding protein or pharmaceutical formulation thereof, and the corticosteroid is administered before the first infusion of the multi-specific binding protein or pharmaceutical formulation thereof. In certain alternative embodiments, where the method includes multiple cycles of treatment, the antihistamine, antipyretic, and corticosteroid are administered at each and every cycle, and within each cycle, the antihistamine, antipyretic, and corticosteroid are administered before each and every infusion of the multi-specific binding protein or pharmaceutical formulation thereof.

Provided herein, in various embodiments, is a method of treating pancreatic cancer in a subject in need thereof including administering a formulation including an effective amount of a multi-specific protein as disclosed herein, in various embodiments, in combination with an anti-PD-1 or an anti-PD-L1 therapy. The formulation can include: (1) a multi-specific binding protein including a first antigen binding site that binds NKG2D, a second antigen binding site that binds EGFR including (i) a VH having the CDR1, CDR2, and CDR3 sequences of SEQ ID NOs: 136, 157, and 138, respectively; and a VL having the CDR1, CDR2, and CDR3 sequences of SEQ ID NOs: 140, 141, and 151, respectively, (ii) a VH having the CDR1, CDR2, and CDR3 sequences of SEQ ID NOs: 136, 146, and 138, respectively, and a VL having the CDR1, CDR2, and CDR3 sequences of SEQ ID NOs: 140, 141, and 142, respectively, (iii) a VH having the CDR1, CDR2, and CDR3 sequences of SEQ ID NOs: 136, 146, and 138, respectively; and a VL having the CDR1, CDR2, and CDR3 sequences of SEQ ID NOs: 140, 141, and 151, respectively, (iv) a VH having the CDR1, CDR2, and CDR3 sequences of SEQ ID NOs: 136, 137, and 138, respectively, and a VL having the CDR1, CDR2, and CDR3 sequences of SEQ ID NOs: 140, 141, and 151, respectively, or (v) a VH having the CDR1, CDR2, and CDR3 sequences of SEQ ID NOs: 136, 137, and 138, respectively, and a VL having the CDR1, CDR2, and CDR3 sequences of SEQ ID NOs: 140, 141, and 142, respectively, and an antibody Fc domain or a portion thereof sufficient to bind CD16, or a third antigen-binding site that binds CD16; and (2) one or more of (i) citrate, (ii) a sugar or sugar alcohol, and (iii) a polysorbate, at pH 6.0 to 7.0. As provided herein, in various embodiments, the anti-PD-1 or the anti-PD-L1 therapy includes nivolumab.

Also provided herein, in various embodiments, is a method of treating pancreatic cancer in a subject in need thereof who has received an effective amount of pre-medication, where the method includes administering a formulation including an effective amount of a multi-specific protein as disclosed herein, in various embodiments, in combination with an anti-PD-1 or an anti-PD-L1 therapy. The formulation can include: (1) a multi-specific binding protein including a first antigen binding site that binds NKG2D, a second antigen binding site that binds EGFR including (i) a VH having the CDR1, CDR2, and CDR3 sequences of SEQ ID NOs: 136, 157, and 138, respectively; and a VL having the CDR1, CDR2, and CDR3 sequences of SEQ ID NOs: 140, 141, and 151, respectively, (ii) a VH having the CDR1, CDR2, and CDR3 sequences of SEQ ID NOs: 136, 146, and 138, respectively, and a VL having the CDR1, CDR2, and CDR3 sequences of SEQ ID NOs: 140, 141, and 142, respectively, (iii) a VH having the CDR1, CDR2, and CDR3 sequences of SEQ ID NOs: 136, 146, and 138, respectively; and a VL having the CDR1, CDR2, and CDR3 sequences of SEQ ID NOs: 140, 141, and 151, respectively, (iv) a VH having the CDR1, CDR2, and CDR3 sequences of SEQ ID NOs: 136, 137, and 138, respectively, and a VL having the CDR1, CDR2, and CDR3 sequences of SEQ ID NOs: 140, 141, and 151, respectively, or (v) a VH having the CDR1, CDR2, and CDR3 sequences of SEQ ID NOs: 136, 137, and 138, respectively, and a VL having the CDR1, CDR2, and CDR3 sequences of SEQ ID NOs: 140, 141, and 142, respectively, and an antibody Fc domain or a portion thereof sufficient to bind CD16, or a third antigen-binding site that binds CD16; and (2) one or more of (i) citrate, (ii) a sugar or sugar alcohol, and (iii) a polysorbate, at pH 6.0 to 7.0. As provided herein, in various embodiments, the anti-PD-1 or the anti-PD-L1 therapy includes nivolumab, and the pre-medication includes an (i) an anti-histamine (e.g., diphenhydramine) and an anti-pyretic (e.g., acetaminophen); and (ii) a corticosteroid (e.g. methylprednisolone). In some embodiments, where the method of treatment includes administering multiple doses of the formulation including a multi-specific binding protein disclosed herein, the antihistamine and antipyretic are administered before each and every infusion of the multi-specific binding protein or pharmaceutical formulation thereof, and the corticosteroid is administered before the first infusion only. In some embodiments, where the method includes multiple cycles of treatment, the antihistamine, antipyretic, and corticosteroid are administered at each and every cycle, and within each cycle, the antihistamine and antipyretic are administered before each and every infusion of the multi-specific binding protein or pharmaceutical formulation thereof, and the corticosteroid is administered before the first infusion of the multi-specific binding protein or pharmaceutical formulation thereof. In certain alternative embodiments, where the method includes multiple cycles of treatment, the antihistamine, antipyretic, and corticosteroid are administered at each and every cycle, and within each cycle, the antihistamine, antipyretic, and corticosteroid are administered before each and every infusion of the multi-specific binding protein or pharmaceutical formulation thereof.

Provided herein, in various embodiments, is a method of treating HNSCC in a subject in need thereof who has received an effective amount of pre-medication, where the method includes administering a formulation including an effective amount of a multi-specific protein as disclosed herein, in various embodiments, in combination with an anti-PD-1 or an anti-PD-L1 therapy. The formulation can include: (1) a multi-specific binding protein including a first antigen binding site that binds NKG2D, a second antigen binding site that binds EGFR including (i) a VH having the CDR1, CDR2, and CDR3 sequences of SEQ ID NOs: 136, 157, and 138, respectively; and a VL having the CDR1, CDR2, and CDR3 sequences of SEQ ID NOs: 140, 141, and 151, respectively, (ii) a VH having the CDR1, CDR2, and CDR3 sequences of SEQ ID NOs: 136, 146, and 138, respectively, and a VL having the CDR1, CDR2, and CDR3 sequences of SEQ ID NOs: 140, 141, and 142, respectively, (iii) a VH having the CDR1, CDR2, and CDR3 sequences of SEQ ID NOs: 136, 146, and 138, respectively; and a VL having the CDR1, CDR2, and CDR3 sequences of SEQ ID NOs: 140, 141, and 151, respectively, (iv) a VH having the CDR1, CDR2, and CDR3 sequences of SEQ ID NOs: 136, 137, and 138, respectively, and a VL having the CDR1, CDR2, and CDR3 sequences of SEQ ID NOs: 140, 141, and 151, respectively, or (v) a VH having the CDR1, CDR2, and CDR3 sequences of SEQ ID NOs: 136, 137, and 138, respectively, and a VL having the CDR1, CDR2, and CDR3 sequences of SEQ ID NOs: 140, 141, and 142, respectively, and an antibody Fc domain or a portion thereof sufficient to bind CD16, or a third antigen-binding site that binds CD16; and (2) one or more of (i) citrate, (ii) a sugar or sugar alcohol, and (iii) a polysorbate, at pH 6.0 to 7.0. As provided herein, in various embodiments, the anti-PD-1 or the anti-PD-L1 therapy includes nivolumab, and the pre-medication includes an (i) an anti-histamine (e.g., diphenhydramine) and an anti-pyretic (e.g., acetaminophen); and (ii) a corticosteroid (e.g. methylprednisolone). In some embodiments, where the method of treatment includes administering multiple doses of the formulation including a multi-specific binding protein disclosed herein, the antihistamine and antipyretic are administered before each and every infusion of the multi-specific binding protein or pharmaceutical formulation thereof, and the corticosteroid is administered before the first infusion only. In some embodiments, where the method includes multiple cycles of treatment, the antihistamine, antipyretic, and corticosteroid are administered at each and every cycle, and within each cycle, the antihistamine and antipyretic are administered before each and every infusion of the multi-specific binding protein or pharmaceutical formulation thereof, and the corticosteroid is administered before the first infusion of the multi-specific binding protein or pharmaceutical formulation thereof. In certain alternative embodiments, where the method includes multiple cycles of treatment, the antihistamine, antipyretic, and corticosteroid are administered at each and every cycle, and within each cycle, the antihistamine, antipyretic, and corticosteroid are administered before each and every infusion of the multi-specific binding protein or pharmaceutical formulation thereof.

Provided herein, in various embodiments, is a method of treating colorectal cancer in a subject in need thereof who has received an effective amount of pre-medication, where the method includes administering a formulation including an effective amount of a multi-specific protein as disclosed herein, in various embodiments, in combination with an anti-PD-1 or an anti-PD-L1 therapy. The formulation can include: (1) a multi-specific binding protein including a first antigen binding site that binds NKG2D, a second antigen binding site that binds EGFR including (i) a VH having the CDR1, CDR2, and CDR3 sequences of SEQ ID NOs: 136, 157, and 138, respectively; and a VL having the CDR1, CDR2, and CDR3 sequences of SEQ ID NOs: 140, 141, and 151, respectively, (ii) a VH having the CDR1, CDR2, and CDR3 sequences of SEQ ID NOs: 136, 146, and 138, respectively, and a VL having the CDR1, CDR2, and CDR3 sequences of SEQ ID NOs: 140, 141, and 142, respectively, (iii) a VH having the CDR1, CDR2, and CDR3 sequences of SEQ ID NOs: 136, 146, and 138, respectively; and a VL having the CDR1, CDR2, and CDR3 sequences of SEQ ID NOs: 140, 141, and 151, respectively, (iv) a VH having the CDR1, CDR2, and CDR3 sequences of SEQ ID NOs: 136, 137, and 138, respectively, and a VL having the CDR1, CDR2, and CDR3 sequences of SEQ ID NOs: 140, 141, and 151, respectively, or (v) a VH having the CDR1, CDR2, and CDR3 sequences of SEQ ID NOs: 136, 137, and 138, respectively, and a VL having the CDR1, CDR2, and CDR3 sequences of SEQ ID NOs: 140, 141, and 142, respectively, and an antibody Fc domain or a portion thereof sufficient to bind CD16, or a third antigen-binding site that binds CD16; and (2) one or more of (i) citrate, (ii) a sugar or sugar alcohol, and (iii) a polysorbate, at pH 6.0 to 7.0. As provided herein, in various embodiments, the anti-PD-1 or the anti-PD-L1 therapy includes nivolumab, and the pre-medication includes an (i) an anti-histamine (e.g., diphenhydramine) and an anti-pyretic (e.g., acetaminophen); and (ii) a corticosteroid (e.g. methylprednisolone). In some embodiments, where the method of treatment includes administering multiple doses of the formulation including a multi-specific binding protein disclosed herein, the antihistamine and antipyretic are administered before each and every infusion of the multi-specific binding protein or pharmaceutical formulation thereof, and the corticosteroid is administered before the first infusion only. In some embodiments, where the method includes multiple cycles of treatment, the antihistamine, antipyretic, and corticosteroid are administered at each and every cycle, and within each cycle, the antihistamine and antipyretic are administered before each and every infusion of the multi-specific binding protein or pharmaceutical formulation thereof, and the corticosteroid is administered before the first infusion of the multi-specific binding protein or pharmaceutical formulation thereof. In certain alternative embodiments, where the method includes multiple cycles of treatment, the antihistamine, antipyretic, and corticosteroid are administered at each and every cycle, and within each cycle, the antihistamine, antipyretic, and corticosteroid are administered before each and every infusion of the multi-specific binding protein or pharmaceutical formulation thereof.

Provided herein, in various embodiments, is a method of treating NSCLC in a subject in need thereof who has received an effective amount of pre-medication, where the method includes administering a formulation including an effective amount of a multi-specific protein as disclosed herein, in various embodiments, in combination with an anti-PD-1 or an anti-PD-L1 therapy. The formulation can include: (1) a multi-specific binding protein including a first antigen binding site that binds NKG2D, a second antigen binding site that binds EGFR including (i) a VH having the CDR1, CDR2, and CDR3 sequences of SEQ ID NOs: 136, 157, and 138, respectively; and a VL having the CDR1, CDR2, and CDR3 sequences of SEQ ID NOs: 140, 141, and 151, respectively, (ii) a VH having the CDR1, CDR2, and CDR3 sequences of SEQ ID NOs: 136, 146, and 138, respectively, and a VL having the CDR1, CDR2, and CDR3 sequences of SEQ ID NOs: 140, 141, and 142, respectively, (iii) a VH having the CDR1, CDR2, and CDR3 sequences of SEQ ID NOs: 136, 146, and 138, respectively; and a VL having the CDR1, CDR2, and CDR3 sequences of SEQ ID NOs: 140, 141, and 151, respectively, (iv) a VH having the CDR1, CDR2, and CDR3 sequences of SEQ ID NOs: 136, 137, and 138, respectively, and a VL having the CDR1, CDR2, and CDR3 sequences of SEQ ID NOs: 140, 141, and 151, respectively, or (v) a VH having the CDR1, CDR2, and CDR3 sequences of SEQ ID NOs: 136, 137, and 138, respectively, and a VL having the CDR1, CDR2, and CDR3 sequences of SEQ ID NOs: 140, 141, and 142, respectively, and an antibody Fc domain or a portion thereof sufficient to bind CD16, or a third antigen-binding site that binds CD16; and (2) one or more of (i) citrate, (ii) a sugar or sugar alcohol, and (iii) a polysorbate, at pH 6.0 to 7.0. As provided herein, in various embodiments, the anti-PD-1 or the anti-PD-L1 therapy includes nivolumab, and the pre-medication includes an (i) an anti-histamine (e.g., diphenhydramine) and an anti-pyretic (e.g., acetaminophen); and (ii) a corticosteroid (e.g. methylprednisolone). In some embodiments, where the method of treatment includes administering multiple doses of the formulation including a multi-specific binding protein disclosed herein, the antihistamine and antipyretic are administered before each and every infusion of the multi-specific binding protein or pharmaceutical formulation thereof, and the corticosteroid is administered before the first infusion only. In some embodiments, where the method includes multiple cycles of treatment, the antihistamine, antipyretic, and corticosteroid are administered at each and every cycle, and within each cycle, the antihistamine and antipyretic are administered before each and every infusion of the multi-specific binding protein or pharmaceutical formulation thereof, and the corticosteroid is administered before the first infusion of the multi-specific binding protein or pharmaceutical formulation thereof. In certain alternative embodiments, where the method includes multiple cycles of treatment, the antihistamine, antipyretic, and corticosteroid are administered at each and every cycle, and within each cycle, the antihistamine, antipyretic, and corticosteroid are administered before each and every infusion of the multi-specific binding protein or pharmaceutical formulation thereof.

Provided herein, in various embodiments, is a method of treating esophageal adenocarcinoma in a subject in need thereof who has received an effective amount of pre-medication, where the method includes administering a formulation including an effective amount of a multi-specific protein as disclosed herein, in various embodiments, in combination with an anti-PD-1 or an anti-PD-L1 therapy. The formulation can include: (1) a multi-specific binding protein including a first antigen binding site that binds NKG2D, a second antigen binding site that binds EGFR including (i) a VH having the CDR1, CDR2, and CDR3 sequences of SEQ ID NOs: 136, 157, and 138, respectively; and a VL having the CDR1, CDR2, and CDR3 sequences of SEQ ID NOs: 140, 141, and 151, respectively, (ii) a VH having the CDR1, CDR2, and CDR3 sequences of SEQ ID NOs: 136, 146, and 138, respectively, and a VL having the CDR1, CDR2, and CDR3 sequences of SEQ ID NOs: 140, 141, and 142, respectively, (iii) a VH having the CDR1, CDR2, and CDR3 sequences of SEQ ID NOs: 136, 146, and 138, respectively; and a VL having the CDR1, CDR2, and CDR3 sequences of SEQ ID NOs: 140, 141, and 151, respectively, (iv) a VH having the CDR1, CDR2, and CDR3 sequences of SEQ ID NOs: 136, 137, and 138, respectively, and a VL having the CDR1, CDR2, and CDR3 sequences of SEQ ID NOs: 140, 141, and 151, respectively, or (v) a VH having the CDR1, CDR2, and CDR3 sequences of SEQ ID NOs: 136, 137, and 138, respectively, and a VL having the CDR1, CDR2, and CDR3 sequences of SEQ ID NOs: 140, 141, and 142, respectively, and an antibody Fc domain or a portion thereof sufficient to bind CD16, or a third antigen-binding site that binds CD16; and (2) one or more of (i) citrate, (ii) a sugar or sugar alcohol, and (iii) a polysorbate, at pH 6.0 to 7.0. As provided herein, in various embodiments, the anti-PD-1 or the anti-PD-L1 therapy includes nivolumab, and the pre-medication includes an (i) an anti-histamine (e.g., diphenhydramine) and an anti-pyretic (e.g., acetaminophen); and (ii) a corticosteroid (e.g. methylprednisolone). In some embodiments, where the method of treatment includes administering multiple doses of the formulation including a multi-specific binding protein disclosed herein, the antihistamine and antipyretic are administered before each and every infusion of the multi-specific binding protein or pharmaceutical formulation thereof, and the corticosteroid is administered before the first infusion only. In some embodiments, where the method includes multiple cycles of treatment, the antihistamine, antipyretic, and corticosteroid are administered at each and every cycle, and within each cycle, the antihistamine and antipyretic are administered before each and every infusion of the multi-specific binding protein or pharmaceutical formulation thereof, and the corticosteroid is administered before the first infusion of the multi-specific binding protein or pharmaceutical formulation thereof. In certain alternative embodiments, where the method includes multiple cycles of treatment, the antihistamine, antipyretic, and corticosteroid are administered at each and every cycle, and within each cycle, the antihistamine, antipyretic, and corticosteroid are administered before each and every infusion of the multi-specific binding protein or pharmaceutical formulation thereof.

In some embodiments, the anti-PD-1 therapy is nivolumab. In some embodiments, nivolumab is administered at dose of 1-1000 mg, 100-1000 mg, 200-1000 mg, 300-1000 mg, 400-1000 mg, 500-1000 mg, 600-1000 mg, 700-1000 mg, 800-1000 mg, 900-1000 mg, 1-800 mg, 100-800 mg, 200-800 mg, 300-800 mg, 400-800 mg, 500-800 mg, 600-800 mg, 700-800 mg, 1-600 mg, 100-600 mg, 200-600 mg, 300-600 mg, 400-600 mg, 500-600 mg, 1-400 mg, 100-400 mg, 200-400 mg, 300-400 mg, 1-200 mg, or 100-200 mg. In some embodiments nivolumab is administered at a dose of about 400 mg, about 420 mg, about 440 mg, about 460 mg, about 480 mg, about 500 mg, about 520 mg, or about 540 mg. In some embodiments, nivolumab is administered at dose of about 480 mg.

In some embodiments, nivolumab is administered intravenously, parentally, or orally. In some embodiments, nivolumab is administered over a 10 to 60 minute intravenous infusion (e.g. 10 to 60 minutes, 10 to 50 minutes, 10 to 40 minutes, 10 to 30 minutes, 10 to 20 minutes, 20 to 60 minutes, 20 to 50 minutes, 20 to 40 minutes, 20 to 30 minutes, 30 to 60 minutes, 30 to 50 minutes, 30 to 40 minutes, 40 to 60 minutes, 40 to 50 minutes, or 50 to 60 minutes). For example, in some embodiments, nivolumab is administered over a 30 minute intravenous infusion. In some embodiments, nivolumab is administered on day 1, day 8, day 15, or day 22 of each treatment cycle. In some embodiments, nivolumab is administered on day 8 of each treatment cycle. In some embodiments, nivolumab is administered once every 4 weeks. In some embodiment Nivolumab is administered with no more than one hour between the end of the nivolumab infusion and the start of the infusion of the multi-specific binding protein.

In some embodiments, the anti-PD-1 therapy is pembrolizumab. In some embodiments, pembrolizumab is administered at dose of 1-1000 mg, 100-1000 mg, 200-1000 mg, 300-1000 mg, 400-1000 mg, 500-1000 mg, 600-1000 mg, 700-1000 mg, 800-1000 mg, 900-1000 mg, 1-800 mg, 100-800 mg, 200-800 mg, 300-800 mg, 400-800 mg, 500-800 mg, 600-800 mg, 700-800 mg, 1-600 mg, 100-600 mg, 200-600 mg, 300-600 mg, 400-600 mg, 500-600 mg, 1-400 mg, 100-400 mg, 200-400 mg, 300-400 mg, 1-200 mg, or 100-200 mg. In some embodiments pembrolizumab is administered at a dose of about 320 mg, about, 340 mg, about 360 mg, about 380 mg, about 400 mg, about 420 mg, about 440 mg, about 460 mg, about 480 mg, about 500 mg, about 520 mg, or about 540 mg. In some embodiments, pembrolizumab is administered at a dose of 400 mg. In some embodiments, pembrolizumab is administered every 1, 2, 3, 4, 5, 6, 7, or 8 weeks. In some embodiments, pembrolizumab is administered every 6 weeks.

In some embodiments, the subject is eligible for anti-PD-1 or an anti-PD-L1 therapy for a malignancy of epithelial origin. In some embodiments, no standard therapy exists, or standard therapy of the subject has failed for a malignancy of epithelial origin. In some embodiments, the subject previously received anti-PD-1 or anti-PD-L1 therapy.

Proteins of the present application can also be used as an adjunct to surgical removal of the primary lesion.

The amount of multi-specific binding protein, or pharmaceutical formulation thereof, and additional therapeutic agent and the relative timing of administration may be selected in order to achieve a desired combined therapeutic effect. For example, when administering a combination therapy to a patient in need of such administration, the therapeutic agents in the combination, or a pharmaceutical composition or compositions including the therapeutic agents, may be administered in any order such as, for example, sequentially, concurrently, together, simultaneously and the like. Further, for example, a multi-specific binding protein, or pharmaceutical formulation thereof, can be administered during a time when the additional therapeutic agent(s) exerts its prophylactic or therapeutic effect, or vice versa.

### VI. Pharmaceutical Formulations

The present disclosure provides pharmaceutical formulations including a multi-specific binding protein disclosed herein. The pharmaceutical formulation includes one or more excipients and is maintained at a certain pH. The term “excipient,” as used herein, means any non-therapeutic agent added to the formulation to provide a desired physical or chemical property, for example, pH, osmolarity, viscosity, clarity, color, isotonicity, odor, sterility, stability, rate of dissolution or release, adsorption, or penetration.

The multi-specific binding proteins of the present disclosure can be formulated in a pharmaceutical formulation at various concentrations. In some embodiments, the pharmaceutical formulation includes greater than or equal to 1 mg/mL, greater than or equal to 10 mg/mL, greater than or equal to 20 mg/mL, greater than or equal to 30 mg/mL, greater than or equal to 40 mg/mL, greater than or equal to 50 mg/mL, greater than or equal to 60 mg/mL, greater than or equal to 70 mg/mL, greater than or equal to 80 mg/mL, greater than or equal to 90 mg/mL, greater than or equal to 100 mg/mL, greater than or equal to 125 mg/mL, greater than or equal to 150 mg/mL, greater than or equal to 175 mg/mL, or greater than or equal to 200 mg/mL of the multi-specific binding protein. In certain embodiments, the pharmaceutical formulation includes 1 mg/ml to 200 mg/ml, 2 mg/ml to 200 mg/ml, 5 mg/ml to 200 mg/ml, 7.5 mg/ml to 200 mg/ml, 10 mg/ml to 200 mg/ml, 12.5 mg/ml to 200 mg/ml, 15 mg/ml to 200 mg/ml, 20 mg/ml to 200 mg/ml, 25 mg/ml to 200 mg/ml, 50 mg/ml to 200 mg/ml, 75 mg/ml to 200 mg/ml, 100 mg/ml to 200 mg/ml, 125 mg/ml to 200 mg/ml, 150 mg/ml to 200 mg/ml, 175 mg/ml to 200 mg/ml, 1 mg/ml to 150 mg/ml, 2 mg/ml to 150 mg/ml, 5 mg/ml to 150 mg/ml, 7.5 mg/ml to 150 mg/ml, 10 mg/ml to 150 mg/ml, 12.5 mg/ml to 150 mg/ml, 15 mg/ml to 150 mg/ml, 20 mg/ml to 150 mg/ml, 25 mg/ml to 150 mg/ml, 50 mg/ml to 150 mg/ml, 75 mg/ml to 150 mg/ml, 100 mg/ml to 150 mg/ml, 125 mg/ml to 150 mg/ml, 1 mg/ml to 100 mg/ml, 2 mg/ml to 100 mg/ml, 5 mg/ml to 100 mg/ml, 7.5 mg/ml to 100 mg/ml, 10 mg/ml to 100 mg/ml, 12.5 mg/ml to 100 mg/ml, 15 mg/ml to 100 mg/ml, 20 mg/ml to 100 mg/ml, 25 mg/ml to 100 mg/ml, 50 mg/ml to 100 mg/ml, 75 mg/ml to 100 mg/ml, 1 mg/ml to 50 mg/ml, 2 mg/ml to 50 mg/ml, 5 mg/ml to 50 mg/ml, 7.5 mg/ml to 50 mg/ml, 10 mg/ml to 50 mg/ml, 12.5 mg/ml to 50 mg/ml, 15 mg/ml to 50 mg/ml, 20 mg/ml to 50 mg/ml, 25 mg/ml to 50 mg/ml, 1 mg/ml to 25 mg/ml, 2 mg/ml to 25 mg/ml, 5 mg/ml to 25 mg/ml, 7.5 mg/ml to 25 mg/ml, 10 mg/ml to 25 mg/ml, 12.5 mg/ml to 25 mg/ml, 15 mg/ml to 25 mg/ml, 20 mg/ml to 25 mg/ml, 1 mg/ml to 20 mg/ml, 2 mg/ml to 20 mg/ml, 5 mg/ml to 20 mg/ml, 7.5 mg/ml to 20 mg/ml, 10 mg/ml to 20 mg/ml, 12.5 mg/ml to 20 mg/ml, or 15 mg/ml to 20 mg/ml of the multi-specific binding protein. In some embodiments, the pharmaceutical formulation includes about 5 mg/ml, about 7.5 mg/ml, about 10 mg/ml. about 12.5 mg/ml, about 15 mg/ml, about 20 mg/ml, about 25 mg/ml, or about 50 mg/ml of the multi-specific binding protein. In certain embodiments, the pharmaceutical formulation includes about 15 mg/ml of the multi-specific binding protein.

### Excipients and pH

The one or more excipients in the pharmaceutical formulation of the present invention includes a buffering agent. The term “buffering agent,” as used herein, refers to one or more components that when added to an aqueous solution is able to protect the solution against variations in pH when adding acid or alkali, or upon dilution with a solvent. In some embodiments, citrate, phosphate buffers, glycinate, carbonate, histidine buffers and the like can be used, in which case, sodium, potassium or ammonium ions can serve as counterions.

In certain embodiments, the buffer or buffer system includes at least one buffer that has a buffering range that overlaps fully or in part with the range of pH 6.0 to 7.0. In certain embodiments, the buffer has a pKa of about 6.0 to 7.0. In certain embodiments, the buffer includes citrate. In certain embodiments, the citrate is present at a concentration of 5 to 100 mM, 7.5 to 100 mM, 10 to 100 mM, 12.5 to 100 mM, 15 to 100 mM, 17.5 to 100 mM, 20 to 100 mM, 22.5 to 100 mM, 25 to 100 mM, 50 mM to 100 mM, 75 mM to 100 mM, 5 to 75 mM, 7.5 to 75 mM, 10 to 75 mM, 12.5 to 75 mM, 15 to 75 mM, 17.5 to 75 mM, 20 to 75 mM, 22.5 to 75 mM, 25 to 75 mM, 50 mM to 75 mM, 5 to 50 mM, 7.5 to 50 mM, 10 to 50 mM, 12.5 to 50 mM, 15 to 50 mM, 17.5 to 50 mM, 20 to 50 mM, 22.5 to 50 mM, 25 to 50 mM, 5 to 25 mM, 7.5 to 25 mM, 10 to 25 mM, 12.5 to 25 mM, 15 to 25 mM, 17.5 to 25 mM, 20 to 25 mM, 5 mM to 20 mM, 7.5 to 20 mM, 10 mM to 20 mM, 12.5 to 20 mM, 15 mM to 20 mM, 17.5 to 20 mM, 5 mM to 15 mM, 7.5 to 15 mM, or 10 mM to 15 mM. In certain embodiments, the citrate is present at a concentration of about 5 mM, about 7.5 mM, about 10 mM, about 12.5 mM, about 15 mM, about 17.5 mM about 20 mM, about 22.5 mM, about 25 mM, or about 50 mM. In certain embodiments, the citrate is present at a concentration of 20 mM.

The pharmaceutical formulation disclosed herein may have a pH of 6.0 to 7.0. For example, in certain embodiments, the pharmaceutical formulation has a pH of 6.1 to 7.0, 6.2 to 7.0, 6.3 to 7.0, 6.4 to 7.0, 6.5 to 7.0, 6.6 to 7.0, 6.7 to 7.0, 6.8 to 7.0, 6.9 to 7.0, 6.1 to 6.9, 6.2 to 6.9, 6.3 to 6.9, 6.4 to 6.9, 6.5 to 6.9, 6.6 to 6.9, 6.7 to 6.9, 6.8 to 6.9, 6.1 to 6.8, 6.2 to 6.8, 6.3 to 6.8, 6.4 to 6.8, 6.5 to 6.8, 6.6 to 6.8, 6.7 to 6.8, 6.1 to 6.7, 6.2 to 6.7, 6.3 to 6.7, 6.4 to 6.7, 6.5 to 6.7, 6.6 to 6.7, 6.1 to 6.6, 6.2 to 6.6, 6.3 to 6.6, 6.4 to 6.6, 6.5 to 6.6, 6.1 to 6.5, 6.2 to 6.5, 6.3 to 6.5, 6.4 to 6.5, 6.1 to 6.4, 6.2 to 6.4, 6.3 to 6.4, 6.1 to 6.3, 6.2 to 6.3, or 6.1 to 6.2. In certain embodiments, the pharmaceutical composition or pharmaceutical formulation has a pH of about 6.2, about 6.3, about 6.4, about 6.5, about 6.6, about 6.7, or about 6.8. In certain embodiments, the pharmaceutical formulation has a pH of about 6.5. Under the rules of scientific rounding, a pH greater than or equal to 5.95 and smaller than or equal to 6.05 is rounded as 6.0.

The one or more excipients in the pharmaceutical formulation disclosed herein may further include a sugar or sugar alcohol. Sugars and sugar alcohols are useful in pharmaceutical formulations as thermal stabilizers. In certain embodiments, the pharmaceutical formulation includes a sugar alcohol, for example, a sugar alcohol derived from a monosaccharide (e.g., mannitol, sorbitol, or xylitol), a sugar alcohol derived from a disaccharide (e.g., lactitol or maltitol), or a sugar alcohol derived from an oligosaccharide. In certain embodiments, the pharmaceutical formulation includes a sugar, for example, a monosaccharide (glucose, xylose, or erythritol), a disaccharide (e.g., sucrose, trehalose, maltose, or galactose), or an oligosaccharide (e.g., stachyose). In specific embodiments, the pharmaceutical formulation includes mannitol.

The amount of the sugar or sugar alcohol contained within the formulation can vary depending on the specific circumstances and intended purposes for which the formulation is used. In certain embodiments, the pharmaceutical formulation includes the sugar or sugar alcohol at 2% to 10% (w/v), 3% to 10% (w/v), 4% to 10% (w/v), 5% to 10% (w/v), 6% to 10% (w/v), 7% to 10% (w/v), 8% to 10% (w/v), 9% to 10% (w/v), 2% to 9% (w/v), 3% to 9% (w/v), 4% to 9% (w/v), 5% to 9% (w/v), 6% to 9% (w/v), 7% to 9% (w/v), 8% to 9% (w/v), 2% to 8% (w/v), 3% to 8% (w/v), 4% to 8% (w/v), 5% to 8% (w/v), 6% to 8% (w/v), 7% to 8% (w/v), 2% to 7% (w/v), 3% to 7% (w/v), 4% to 7% (w/v), 5% to 7% (w/v), 6% to 7% (w/v), 2% to 6% (w/v), 3% to 6% (w/v), 4% to 6% (w/v), 5% to 6% (w/v), 2% to 5% (w/v), 3% to 5% (w/v), 4% to 5% (w/v), 2% to 4% (w/v), 3% to 4% (w/v), or 2% to 3% (w/v).

In certain embodiments, the pharmaceutical formulation, includes mannitol at 4% to 8% (w/v), 5% to 8% (w/v), 6% to 8% (w/v), 7% to 8% (w/v), 4% to 7% (w/v), 5% to 7% (w/v), 6% to 7% (w/v), 4% to 6% (w/v), or 4% to 5% (w/v). In certain embodiments, the pharmaceutical formulation includes about 4%, about 5%, about 6%, about 7%, or about 8% mannitol (w/v). In certain embodiments, the pharmaceutical formulation includes about 6% mannitol (w/v).

The one or more excipients in the pharmaceutical formulation disclosed herein further includes a surfactant. The term “surfactant,” as used herein, refers to a surface active molecule containing both a hydrophobic portion (e.g., alkyl chain) and a hydrophilic portion (e.g., carboxyl and carboxylate groups). Surfactants are useful in pharmaceutical formulations for reducing aggregation of a therapeutic protein. Surfactants suitable for use in the pharmaceutical formulations are generally non-ionic surfactants and include, but are not limited to, polysorbates (e.g. polysorbates 20 or 80); poloxamers (e.g. poloxamer 188); sorbitan esters and derivatives; Triton; sodium laurel sulfate; sodium octyl glycoside; lauryl-, myristyl-, linoleyl-, or stearyl-sulfobetadine; lauryl-, myristyl-, linoleyl- or stearyl-sarcosine; linoleyl-, myristyl-, or cetyl-betaine; lauramidopropyl-cocamidopropyl-, linoleamidopropyl-, myristamidopropyl-, palmidopropyl-, or isostearamidopropylbetaine (e.g., lauroamidopropyl); myristamidopropyl-, palmidopropyl-, or isostearamidopropyl-dimethylamine; sodium methyl cocoyl-, or disodium methyl oleyl-taurate; and the MONAQUAT™ series (Mona Industries, Inc., Paterson, N.J.), polyethylene glycol, polypropyl glycol, and copolymers of ethylene and propylene glycol (e.g., Pluronics, PF68 etc.). In certain embodiments, the surfactant is a polysorbate. In certain embodiments, the surfactant is polysorbate 80.

The amount of a non-ionic surfactant contained within the pharmaceutical composition or pharmaceutical formulation of the present invention may vary depending on the specific properties desired of the formulation, as well as the particular circumstances and purposes for which the formulations are intended to be used. In certain embodiments, the pharmaceutical formulation includes 0.005% to 0.5% (w/v), 0.005% to 0.25% (w/v), 0.005% to 0.2% (w/v), 0.005% to 0.1% (w/v), 0.005% to 0.05% (w/v), 0.005% to 0.025% (w/v), 0.005% to 0.02% (w/v), 0.005% to 0.01% (w/v), 0.0075% to 0.5% (w/v), 0.0075% to 0.2% (w/v), 0.0075% to 0.25% (w/v), 0.0075% to 0.1% (w/v), 0.0075% to 0.05% (w/v), 0.0075% to 0.025% (w/v), 0.0075% to 0.02% (w/v), 0.0075% to 0.01% (w/v), 0.01% to 0.5% (w/v), 0.01% to 0.25% (w/v), 0.01% to 0.2% (w/v), 0.01% to 0.1% (w/v), 0.01% to 0.05% (w/v), 0.01% to 0.025% (w/v), or 0.01% to 0.02% (w/v) of the non-ionic surfactant (e.g., polysorbate 80). In certain embodiments, the pharmaceutical formulation includes 0.005% (w/v), 0.01% (w/v), 0.02% (w/v), 0.03% (w/v), 0.04% (w/v), 0.05% (w/v), 0.06% (w/v), 0.07% (w/v), 0.08% (w/v), 0.09% (w/v), 0.1% (w/v), 0.15% (w/v), 0.2% (w/v), 0.25% (w/v), 0.3% (w/v), 0.35% (w/v), 0.4% (w/v), 0.45% (w/v), or 0.5% (w/v) of polysorbate 80. In certain embodiments, the pharmaceutical formulation includes about 0.01% (w/v) polysorbate 80.

In certain embodiments, the pharmaceutical formulation is isotonic. An “isotonic” formulation is one which has essentially the same osmotic pressure as human blood. Isotonic formulations generally have an osmotic pressure from about 250 to 350 mOsmol/kgH₂O. Isotonicity can be measured using a vapor pressure or ice-freezing type osmometer. In certain embodiments, the osmolarity of the pharmaceutical composition or pharmaceutical formulation is 250 to 350 mOsmol/kgH₂O. In certain embodiments, the osmolarity of the pharmaceutical composition or pharmaceutical formulation is 300 to 350 mOsmol/kgH₂O. Substances such as sugar, sugar alcohol, and NaCl can be included in the pharmaceutical formulation for desired osmolarity.

The pharmaceutical formulation disclosed herein may further include one or more other substances, such as a bulking agent or a preservative. A “bulking agent” is a compound which adds mass to a lyophilized mixture and contributes to the physical structure of the lyophilized cake (e.g., facilitates the production of an essentially uniform lyophilized cake which maintains an open pore structure). Illustrative bulking agents include mannitol, glycine, polyethylene glycol and sorbitol. The lyophilized formulations of the present invention may contain such bulking agents. A preservative reduces bacterial action and may, for example, facilitate the production of a multi-use (multiple-dose) formulation.

### Exemplary Formulations

In some embodiments, the pharmaceutical formulation of the present invention includes a multi-specific binding protein, and one or more of: citrate; a sugar or sugar alcohol; and a polysorbate, at pH 6.0 to 7.0. In some embodiments, the pharmaceutical formulation of the present invention includes the multi-specific binding protein, citrate, a sugar or sugar alcohol, and a polysorbate, at pH 6.0 to 7.0. Also provided in the present disclosure are any one of the formulations above, further including one or more of: (a) citrate, (b) a sugar or sugar alcohol, and (c) a polysorbate.

In some embodiments, the present disclosure provides formulations consisting essentially of: (a) a multi-specific protein as described herein, (b) citrate, (c) a sugar or sugar alcohol, and (d) a polysorbate, at pH 6.0 to 7.0. The concentration of each of the components in that formulation can be any one of the concentrations or ranges as described in the present disclosure.

In some embodiments, the concentration of the multi-specific binding protein in the pharmaceutical formulation is 1 mg/mL to 125 mg/mL, 2 mg/mL to 100 mg/mL, 5 mg/mL to 50 mg/mL, 5 mg/mL to 20 mg/mL, or 10 mg/mL to 20 mg/mL. In some embodiments, the concentration of the multi-specific binding protein in the pharmaceutical formulation is about 15 mg/mL. In some embodiments, the formulation is diluted with a suitable diluent in the range of 1:0 to 1:10 prior to administration to a subject. In some embodiments, the concentration of citrate in the pharmaceutical formulation is 15 mM to 25 mM or 17.5 mM to 22.5 mM. In some embodiments, pharmaceutical formulations of the present disclosure contain about 20 mM citrate. In some embodiments, the formulation also contains a sugar alcohol of a monosaccharide. In some embodiments, the sugar alcohol is mannitol. In some embodiments, the concentration of mannitol is 4% to 8% (w/v) or 5% to 7% (w/v). In some embodiments, the concentration of mannitol is about 6% (w/v). In some embodiments, the formulation includes a polysorbate and the polysorbate is 80. In some embodiments, the concentration of polysorbate 80 is 0.005% to 0.05% (w/v) or 0.0075% to 0.025% (w/v). In some embodiments, the concentration of polysorbate 80 is about 0.01% (w/v). In some embodiments, the pH of the formulation is 6.2 to 6.8, or 6.4 to 6.6. In some embodiments, the pH of the formulation is about 6.5. In some embodiments, the formulation contains: (a) 5 mg/mL to 50 mg/mL of the multi-specific binding protein, (b) 15 mM to 25 mM citrate, (c) 4% to 8% (w/v) mannitol, and 0.005% to 0.05% (w/v) polysorbate 80. In some embodiments, the formulation is at pH 6.2 to 6.8. In some embodiments, the formulation contains: (a) 10 mg/mL to 20 mg/mL of the multi-specific binding protein, (b) 17.5 mM to 22.5 mM citrate, (c) 5% to 7% (w/v) mannitol, and 0.0075% to 0.025% (w/v) polysorbate 80. In some embodiments, the formulation is at pH 6.4 to 6.6. In some embodiments, the formulation contains: (a) 15 mg/mL of the multi-specific binding protein, (b) 20 mM citrate, (c) 6% (w/v) mannitol, and (d) 0.01% (w/v) polysorbate 80. In some embodiments, the formulation is at about pH 6.5.

Some pharmaceutical formulations of the present disclosure contain:

(a) 5 mg/mL to 50 mg/mL of a multi-specific binding protein;

(b) 15 mM to 25 mM citrate;

(c) 4% to 8% (w/v) mannitol; and

(d) 0.005% to 0.05% (w/v) polysorbate 80,

at pH 6.2 to 6.8.

Some pharmaceutical formulations of the present disclosure consist essentially of:

(a) 5 mg/mL to 50 mg/mL of a multi-specific binding protein;

(b) 15 mM to 25 mM citrate;

(c) 4% to 8% (w/v) mannitol; and

(d) 0.005% to 0.05% (w/v) polysorbate 80,

at pH 6.2 to 6.8.

Some pharmaceutical formulations of the present disclosure contain:

(a) 10 mg/mL to 20 mg/mL of the multi-specific binding protein;

(b) 17.5 mM to 22.5 mM citrate;

(c) 5% to 7% (w/v) mannitol; and

(d) 0.0075% to 0.025% (w/v) polysorbate 80,

at pH 6.4 to 6.6.

Some pharmaceutical formulations of the present disclosure consist essentially of:

(a) 10 mg/mL to 20 mg/mL of the multi-specific binding protein;

(b) 17.5 mM to 22.5 mM citrate;

(c) 5% to 7% (w/v) mannitol; and

(d) 0.0075% to 0.025% (w/v) polysorbate 80,

at pH 6.4 to 6.6.

Some pharmaceutical formulations of the present disclosure consist essentially of:

(a) 15 mg/mL of the multi-specific binding protein;

(b) 20 mM citrate;

(c) 6% (w/v) mannitol; and

(d) 0.01% (w/v) polysorbate 80,

at about pH 6.5.

Some pharmaceutical formulations of the present disclosure contain:

(a) 15 mg/mL of the multi-specific binding protein;

(b) 20 mM citrate;

(c) 6% (w/v) mannitol; and

(d) 0.01% (w/v) polysorbate 80,

at about pH 6.5.

Some pharmaceutical formulations of the present disclosure contain:

(a) a multi-specific binding protein incorporating:
(i) a Fab including a heavy chain variable domain (VH) and a light chain variable domain (VL) that bind NKG2D;
(ii) a single-chain variable fragment (scFv) including a VH and a VL that bind EGFR; and
(iii) an antibody Fc domain, and

(b) one or more of:
(i) 15 mM to 25 mM citrate; and
(ii) 4% to 8% (w/v) mannitol,

at pH 6.0 to 7.0.

Some pharmaceutical formulations of the present disclosure contain:

(i) 10 mg/ml to 20 mg/ml of a multi-specific binding protein incorporating:
(a) a Fab with a VH having the CDR1, CDR2, and CDR3 sequences of SEQ ID NOs: 81, 82, and 112, respectively; and a VL having the CDR1, CDR2, and CDR3 sequences of SEQ ID NOs: 86, 77, and 87, respectively;
(b) an scFv with:
   1) a VH having the CDR1, CDR2, and CDR3 sequences of SEQ ID NOs: 136, 146, and 138, respectively; and a VL having the CDR1, CDR2, and CDR3 sequences of SEQ ID NOs: 140, 141, and 142, respectively, or
   2) a VH having the CDR1, CDR2, and CDR3 sequences of SEQ ID NOs: 136, 157, and 138, respectively; and a VL having the CDR1, CDR2, and CDR3 sequences of SEQ ID NOs: 140, 141, and 151, respectively; and
(c) an antibody Fc domain or a portion thereof sufficient to bind CD16, or a third antigen-binding site that binds CD16;

(ii) 15 mM to 25 mM citrate;

(iii) 4% to 8% (w/v) mannitol; and

(iv) 0.005% to 0.05% (w/v) polysorbate 80,

at pH 6.2 to 6.8.

Some pharmaceutical formulations of the present disclosure contain:

(i) about 15 mg/ml of a multi-specific binding protein incorporating:
(a) a Fab with a VH having the CDR1, CDR2, and CDR3 sequences of SEQ ID NOs: 81, 82, and 97, respectively; and a VL having the CDR1, CDR2, and CDR3 sequences of SEQ ID NOs: 86, 77, and 87, respectively;
(b) an scFv with a VH having the CDR1, CDR2, and CDR3 sequences of SEQ ID NOs: 136, 146, and 138, respectively; and a VL having the CDR1, CDR2, and CDR3 sequences of SEQ ID NOs: 140, 141, and 142, respectively; and
(c) an antibody Fc domain with a first antibody Fc polypeptide linked to the Fab and a second antibody Fc polypeptide linked to the scFv; the first antibody Fc polypeptide is a human IgG1 Fc polypeptide incorporating K360E and K409W substitutions, and the second antibody Fc polypeptide is a human IgG1 Fc polypeptide incorporating Q347R, D399V, and F405T substitutions, numbered according to the EU index.

(ii) about 20 mM citrate;

(iii) about 6% (w/v) mannitol; and

(iv) about 0.01% (w/v) polysorbate 80,

at about pH 6.5.

Some pharmaceutical formulations of the present disclosure contain:

(i) about 15 mg/ml of a multi-specific binding protein incorporating:
(a) a Fab including a VH having the CDR1, CDR2, and CDR3 sequences of SEQ ID NOs: 81, 82, and 97, respectively; and a VL having the CDR1, CDR2, and CDR3 sequences of SEQ ID NOs: 86, 77, and 87, respectively;
(b) an scFv including a VH having the CDR1, CDR2, and CDR3 sequences of SEQ ID NOs: 136, 146, and 138, respectively; and a VL having the CDR1, CDR2, and CDR3 sequences of SEQ ID NOs: 140, 141, and 151, respectively; and
(c) an antibody Fc domain including a first antibody Fc polypeptide linked to the Fab and a second antibody Fc polypeptide linked to the scFv; the first antibody Fc polypeptide is a human IgG1 Fc polypeptide incorporating K360E and K409W substitutions, and the second antibody Fc polypeptide is a human IgG1 Fc polypeptide incorporating Q347R, D399V, and F405T substitutions, numbered according to the EU index.

(ii) about 20 mM citrate;

(iii) about 6% (w/v) mannitol; and

(iv) about 0.01% (w/v) polysorbate 80,

at about pH 6.5.

Some pharmaceutical formulations of the present disclosure contain:

(i) about 15 mg/ml of a multi-specific binding protein incorporating:
(a) a Fab including a VH having the CDR1, CDR2, and CDR3 sequences of SEQ ID NOs: 81, 82, and 97, respectively; and a VL having the CDR1, CDR2, and CDR3 sequences of SEQ ID NOs: 86, 77, and 87, respectively;
(b) an scFv including a VH having the CDR1, CDR2, and CDR3 sequences of SEQ ID NOs: 136, 137, and 138, respectively; and a VL having the CDR1, CDR2, and CDR3 sequences of SEQ ID NOs: 140, 141, and 151, respectively; and
(c) an antibody Fc domain including a first antibody Fc polypeptide linked to the Fab and a second antibody Fc polypeptide linked to the scFv; the first antibody Fc polypeptide is a human IgG1 Fc polypeptide incorporating K360E and K409W substitutions, and the second antibody Fc polypeptide is a human IgG1 Fc polypeptide incorporating Q347R, D399V, and F405T substitutions, numbered according to the EU index.

(ii) about 20 mM citrate;

(iii) about 6% (w/v) mannitol; and

(iv) about 0.01% (w/v) polysorbate 80,

at about pH 6.5.

Some pharmaceutical formulations of the present disclosure contain:

(i) about 15 mg/ml of a multi-specific binding protein incorporating:
(a) a Fab including a VH having the CDR1, CDR2, and CDR3 sequences of SEQ ID NOs: 81, 82, and 97, respectively; and a VL having the CDR1, CDR2, and CDR3 sequences of SEQ ID NOs: 86, 77, and 87, respectively;
(b) an scFv including a VH having the CDR1, CDR2, and CDR3 sequences of SEQ ID NOs: 136, 137, and 138, respectively; and a VL having the CDR1, CDR2, and CDR3 sequences of SEQ ID NOs: 140, 141, and 142, respectively; and
(c) an antibody Fc domain including a first antibody Fc polypeptide linked to the Fab and a second antibody Fc polypeptide linked to the scFv; the first antibody Fc polypeptide is a human IgG1 Fc polypeptide incorporating K360E and K409W substitutions, and the second antibody Fc polypeptide is a human IgG1 Fc polypeptide incorporating Q347R, D399V, and F405T substitutions, numbered according to the EU index.

(ii) about 20 mM citrate;

(iii) about 6% (w/v) mannitol; and

(iv) about 0.01% (w/v) polysorbate 80,

at about pH 6.5.

Some pharmaceutical formulations of the present disclosure contain:

(i) 10 mg/ml to 20 mg/ml of a multi-specific binding protein incorporating:
(a) a Fab including a VH with the amino acid sequence of SEQ ID NO:110 and a VL with the an amino acid sequence of SEQ ID NO:85;
(b) an scFv including:
   1) a VH with the an amino acid sequence of SEQ ID NO:145; and a VL with the an amino acid sequence of SEQ ID NO:147, or
   2) a VH with the an amino acid sequence of SEQ ID NO:156; and a VL with the an amino acid sequence of SEQ ID NO:150; and
(c) an antibody Fc domain or a portion thereof sufficient to bind CD16, or a third antigen-binding site that binds CD16;

(ii) 15 mM to 25 mM citrate;

(iii) 4% to 8% (w/v) mannitol; and

(iv) 0.005% to 0.05% (w/v) polysorbate 80,

at pH 6.2 to 6.8.

Some pharmaceutical formulations of the present disclosure contain:

(i) about 15 mg/ml of a multi-specific binding protein incorporating:
(a) a Fab including a VH with the amino acid sequence of SEQ ID NO:95 and a VL with the amino acid sequence of SEQ ID NO:85;
(b) an scFv including a VH with the amino acid sequence at least 90% identical to SEQ ID NO:145 and a VL with the amino acid sequence at least 90% identical to SEQ ID NO:147; and
(c) an antibody Fc domain including a first antibody Fc polypeptide linked to the Fab and a second antibody Fc polypeptide linked to the scFv; the first antibody Fc polypeptide is a human IgG1 Fc polypeptide incorporating K360E and K409W substitutions, and the second antibody Fc polypeptide is a human IgG1 Fc polypeptide incorporating Q347R, D399V, and F405T substitutions, numbered according to the EU index;

(ii) about 20 mM citrate;

(iii) about 6% (w/v) mannitol; and

(iv) about 0.01% (w/v) polysorbate 80,

at about pH 6.5.

Some pharmaceutical formulations of the present disclosure contain:

(i) about 15 mg/ml of a multi-specific binding protein incorporating:
(a) a Fab including a VH with the amino acid sequence of SEQ ID NO:95 and a VL with the amino acid sequence of SEQ ID NO:85;
(b) an scFv including a VH with the amino acid sequence of SEQ ID NO:170 and a VL with the amino acid sequence of SEQ ID NO:171; and
(c) an antibody Fc domain including a first antibody Fc polypeptide linked to the Fab and a second antibody Fc polypeptide linked to the scFv; the first antibody Fc polypeptide is a human IgG1 Fc polypeptide incorporating K360E and K409W substitutions, and the second antibody Fc polypeptide is a human IgG1 Fc polypeptide incorporating Q347R, D399V, and F405T substitutions, numbered according to the EU index;

(ii) about 20 mM citrate;

(iii) about 6% (w/v) mannitol; and

(iv) about 0.01% (w/v) polysorbate 80,

at about pH 6.5.

Some pharmaceutical formulations of the present disclosure contain:

(i) about 15 mg/ml of a multi-specific binding protein incorporating:
(a) a Fab including a VH having the amino acid sequence of SEQ ID NO:95; and a VL having the amino acid sequence of SEQ ID NO:85;
(b) an scFv including a VH with an amino acid sequence at least 90% identical to SEQ ID NO:135; and a VL with an amino acid sequence at least 90% identical to SEQ ID NO:150; and
(c) an antibody Fc domain including a first antibody Fc polypeptide linked to the Fab and a second antibody Fc polypeptide linked to the scFv; the first antibody Fc polypeptide is a human IgG1 Fc polypeptide incorporating K360E and K409W substitutions, and the second antibody Fc polypeptide is a human IgG1 Fc polypeptide incorporating Q347R, D399V, and F405T substitutions, numbered according to the EU index.

(ii) about 20 mM citrate;

(iii) about 6% (w/v) mannitol; and

(iv) about 0.01% (w/v) polysorbate 80,

at about pH 6.5.

Some pharmaceutical formulations of the present disclosure contain:

(i) about 15 mg/ml of a multi-specific binding protein incorporating:
(a) a Fab including a VH with the amino acid sequence of SEQ ID NO:95 and a VL with the amino acid sequence of SEQ ID NO:85;
(b) an scFv including a VH with an amino acid sequence at least 90% identical to SEQ ID NO:135 and a VL with an amino acid sequence at least 90% identical to SEQ ID NO:139; and
(c) an antibody Fc domain including a first antibody Fc polypeptide linked to the Fab and a second antibody Fc polypeptide linked to the scFv; the first antibody Fc polypeptide is a human IgG1 Fc polypeptide incorporating K360E and K409W substitutions, and the second antibody Fc polypeptide is a human IgG1 Fc polypeptide incorporating Q347R, D399V, and F405T substitutions, numbered according to the EU index.

(ii) about 20 mM citrate;

(iii) about 6% (w/v) mannitol; and

(iv) about 0.01% (w/v) polysorbate 80,

at about pH 6.5.

Some pharmaceutical formulations of the present disclosure contain:

(i) 10 mg/ml to 20 mg/ml of a multi-specific binding protein incorporating:
(a) a Fab including a VH with the amino acid sequence of SEQ ID NO:110 and a VL with the amino acid sequence of SEQ ID NO:85;
(b) an scFv including:
   1) the amino acid sequence of SEQ ID NO:148, or
   2) the amino acid sequence of SEQ ID NO:158; and
(c) an antibody Fc domain or a portion thereof sufficient to bind CD16, or a third antigen-binding site that binds CD16;

(ii) 15 mM to 25 mM citrate;

(iii) 4% to 8% (w/v) mannitol; and

(iv) 0.005% to 0.05% (w/v) polysorbate 80,

at pH 6.2 to 6.8.

Some pharmaceutical formulations of the present disclosure contain:

(i) about 15 mg/ml of a multi-specific binding protein incorporating:
(a) a Fab including a VH with the amino acid sequence of SEQ ID NO:95 and a VL with the amino acid sequence of SEQ ID NO:85;
(b) an scFv with the an amino acid sequence of SEQ ID NO:148; and
(c) an antibody Fc domain including a first antibody Fc polypeptide linked to the Fab and a second antibody Fc polypeptide linked to the scFv; the first antibody Fc polypeptide is a human IgG1 Fc polypeptide incorporating K360E and K409W substitutions, and the second antibody Fc polypeptide is a human IgG1 Fc polypeptide incorporating Q347R, D399V, and F405T substitutions, numbered according to the EU index;

(ii) about 20 mM citrate;

(iii) about 6% (w/v) mannitol; and

(iv) about 0.01% (w/v) polysorbate 80,

at about pH 6.5.

Some pharmaceutical formulations of the present disclosure contain:

(i) about 15 mg/ml of a multi-specific binding protein incorporating:
(a) a Fab including a VH with the amino acid sequence of SEQ ID NO:95; and a VL with the amino acid sequence of SEQ ID NO:85;
(b) an scFv with the amino acid sequence of SEQ ID NO:152; and
(c) an antibody Fc domain including a first antibody Fc polypeptide linked to the Fab and a second antibody Fc polypeptide linked to the scFv; the first antibody Fc polypeptide is a human IgG1 Fc polypeptide incorporating K360E and K409W substitutions, and the second antibody Fc polypeptide is a human IgG1 Fc polypeptide incorporating Q347R, D399V, and F405T substitutions, numbered according to the EU index;

(ii) about 20 mM citrate;

(iii) about 6% (w/v) mannitol; and

(iv) about 0.01% (w/v) polysorbate 80,

at about pH 6.5.

Some pharmaceutical formulations of the present disclosure contain:

(i) about 15 mg/ml of a multi-specific binding protein incorporating:
(a) a Fab including a VH with the amino acid sequence of SEQ ID NO:95 and a VL with the amino acid sequence of SEQ ID NO:85;
(b) an scFv with the amino acid sequence of SEQ ID NO:154; and
(c) an antibody Fc domain including a first antibody Fc polypeptide linked to the Fab and a second antibody Fc polypeptide linked to the scFv; the first antibody Fc polypeptide is a human IgG1 Fc polypeptide incorporating K360E and K409W substitutions, and the second antibody Fc polypeptide is a human IgG1 Fc polypeptide incorporating Q347R, D399V, and F405T substitutions, numbered according to the EU index.

(ii) about 20 mM citrate;

(iii) about 6% (w/v) mannitol; and

(iv) about 0.01% (w/v) polysorbate 80,

at about pH 6.5.

Some pharmaceutical formulations of the present disclosure contain:

(i) about 15 mg/ml of a multi-specific binding protein incorporating:
(a) a Fab including a VH with the amino acid sequence of SEQ ID NO:95 and a VL with the amino acid sequence of SEQ ID NO:85;
(b) an scFv with the amino acid sequence of SEQ ID NO:143; and
(c) an antibody Fc domain including a first antibody Fc polypeptide linked to the Fab and a second antibody Fc polypeptide linked to the scFv; the first antibody Fc polypeptide is a human IgG1 Fc polypeptide incorporating K360E and K409W substitutions, and the second antibody Fc polypeptide is a human IgG1 Fc polypeptide incorporating Q347R, D399V, and F405T substitutions, numbered according to the EU index.

(ii) about 20 mM citrate;

(iii) about 6% (w/v) mannitol; and

(iv) about 0.01% (w/v) polysorbate 80,

at about pH 6.5.

Some pharmaceutical formulations of the present disclosure contain:

(i) about 15 mg/ml of a multi-specific binding protein incorporating:
(a) a first polypeptide with the amino acid sequence of SEQ ID NO:166;
(b) a second polypeptide with the amino acid sequence of SEQ ID NO:164; and
(c) a third polypeptide with the amino acid sequence of SEQ ID NO:165;

(ii) about 20 mM citrate;

(iii) about 6% (w/v) mannitol; and

(iv) about 0.01% (w/v) polysorbate 80,

at about pH 6.5.

Some pharmaceutical formulations of the present disclosure contain:

(i) about 15 mg/ml of a multi-specific binding protein incorporating:
(a) a first polypeptide with the amino acid sequence of SEQ ID NO:167;
(b) a second polypeptide with the amino acid sequence of SEQ ID NO:164; and
(c) a third polypeptide with the amino acid sequence of SEQ ID NO:165;

(ii) about 20 mM citrate;

(iii) about 6% (w/v) mannitol; and

(iv) about 0.01% (w/v) polysorbate 80,

at about pH 6.5.

Some pharmaceutical formulations of the present disclosure contain:

(i) about 15 mg/ml of a multi-specific binding protein incorporating:
(a) a first polypeptide with the amino acid sequence of SEQ ID NO:168;
(b) a second polypeptide with the amino acid sequence of SEQ ID NO:164; and
(c) a third polypeptide with the amino acid sequence of SEQ ID NO:165;

(ii) about 20 mM citrate;

(iii) about 6% (w/v) mannitol; and

(iv) about 0.01% (w/v) polysorbate 80,

at about pH 6.5.

Some pharmaceutical formulations of the present disclosure contain:

(i) about 15 mg/ml of a multi-specific binding protein incorporating:
(a) a first polypeptide with the amino acid sequence of SEQ ID NO:172;
(b) a second polypeptide with the amino acid sequence of SEQ ID NO:164; and
(c) a third polypeptide with the amino acid sequence of SEQ ID NO:165;

(ii) about 20 mM citrate;

(iii) about 6% (w/v) mannitol; and

(iv) about 0.01% (w/v) polysorbate 80,

at about pH 6.5.

### Stability of the Multi-Specific Binding Protein

The pharmaceutical formulations disclosed herein exhibit high levels of stability. A pharmaceutical formulation is stable when the multi-specific binding protein within the formulation retains an acceptable physical property, chemical structure, and/or biological function after storage under defined conditions.

Stability can be measured by determining the percentage of the multi-specific binding protein in the formulation that remains in a native conformation after storage for a defined amount of time at a defined temperature. The percentage of a protein in a native conformation can be determined by, for example, size exclusion chromatography (e.g., size exclusion high performance liquid chromatography), where a protein in the native conformation is not aggregated (eluted in a high molecular weight fraction) or degraded (eluted in a low molecular weight fraction). In certain embodiments, more than 95%, 96%, 97%, 98%, or 99% of the multi-specific binding protein has native conformation, as determined by size-exclusion chromatography, after incubation at 4° C. for 3 weeks. In certain embodiments, more than 90%, 91%, 92%, 93%, 94%, 95%, 96%, 97%, 98%, or 99% of the multi-specific binding protein has native conformation, as determined by size-exclusion chromatography, after incubation at 50° C. for 3 weeks. In certain embodiments, less than 0.5%, 0.6%, 0.7%, 0.8%, 0.9%, or 1% of the multi-specific binding protein forms a high molecular weight complex (i.e., having a higher molecular weight than the native protein), as determined by size-exclusion chromatography, after incubation at 4° C. for 3 weeks. In certain embodiments, less than or equal to 1%, 2%, 3%, 4%, or 5% of the multi-specific binding protein form a high molecular weight complex (i.e., having a higher molecular weight than the native protein), as determined by size-exclusion chromatography, after incubation at 50° C. for 3 weeks. In certain embodiments, less than or equal to 0.5%, 0.6%, 0.7%, 0.8%, 0.9%, or 1% of the multi-specific binding protein is degraded (i.e., having a lower molecular weight than the native protein), as determined by size-exclusion chromatography, after incubation at 4° C. for 3 weeks. In certain embodiments, less than or equal to 1%, 1.5%, 2%, 2.5%, or 3% of the multi-specific binding protein is degraded (i.e., having a lower molecular weight than the native protein), as determined by size-exclusion chromatography, after incubation at 50° C. for 3 weeks.

Stability can also be measured by determining the percentage of multi-specific binding protein present in a more acidic fraction (“acidic form”) relative to the main fraction of protein (“main charge form”). While not wishing to be bound by theory, deamidation of a protein may cause it to become more negatively charged and thus more acidic relative to the non-deamidated protein (see, e.g., Robinson, Protein Deamidation, (2002) PNAS 99(8):5283-88). The percentage of the acidic form of a protein can be determined by ion exchange chromatography (e.g., cation exchange high performance liquid chromatography) or imaged capillary isoelectric focusing (icIEF). In certain embodiments, at least 50%, 60%, 70%, 80%, or 90% of the multi-specific binding protein in the pharmaceutical formulation is in the main charge form after incubation at 4° C. for 3 weeks. In certain embodiments, at least 15%, 20%, 25%, 30%, 40%, 50%, 60%, 70%, 80%, or 90% of the multi-specific binding protein in the pharmaceutical formulation is in the main charge form after incubation at 50° C. for 3 weeks. In certain embodiments, no more than 4%, 20%, 30%, 40%, or 50% of the multi-specific binding protein in the pharmaceutical formulation is in an acidic form after incubation at 4° C. for 3 weeks. In certain embodiments, no more than 4%, 20%, 30%, 40%, 50%, 60%, 70%, 75%, 80%, or 85% of the multi-specific binding protein in the pharmaceutical formulation is in an acidic form after incubation at 50° C. for 3 weeks.

Stability can also be measured by determining the purity of the multi-specific binding protein by electrophoresis after denaturing the protein with sodium dodecyl sulfate (SDS). The protein sample can be denatured in the presence or absence of an agent that reduces protein disulfide bonds (e.g., β-mercaptoethanol). In certain embodiments, the purity of the multi-specific binding protein in the pharmaceutical formulation, as measured by capillary electrophoresis after denaturing the protein sample under reducing conditions (e.g., in the presence of β-mercaptoethanol), is at least 95%, 96%, 97%, 98%, or 99% after incubation at 4° C. for 3 weeks. In certain embodiments, the purity of the multi-specific binding protein in the pharmaceutical formulation, as measured by capillary electrophoresis after denaturing the protein sample under reducing conditions (e.g., in the presence of β-mercaptoethanol), is at least 90%, 91%, 92%, 93%, 94%, 95%, 96%, 97%, 98%, or 99% after incubation at 50° C. for 3 weeks. In certain embodiments, the purity of the multi-specific binding protein in the pharmaceutical formulation, as measured by capillary electrophoresis after denaturing the protein sample under non-reducing conditions, is at least 95%, 96%, 97%, 98%, or 99% after incubation at 4° C. for 3 weeks. In certain embodiments, the purity of the multi-specific binding protein in the pharmaceutical formulation, as measured by capillary electrophoresis after denaturing the protein sample under non-reducing conditions, is at least 85%, 86%, 87%, 88%, 89%, 90%, 91%, 92%, 93%, 94%, 95%, 96%, 97%, 98%, or 99% after incubation at 50° C. for 3 weeks.

Stability can also be measured by determining the parameters of a protein solution by dynamic light scattering. The Z-average and polydispersity index (PDI) values indicate the average diameter of particles in a solution and these measures increase when aggregates are present in the solution. The monomer % Pd value indicates the spread of different monomers detected, where lower values indicate a monodisperse solution, which is preferred. The monomer size detected by DLS is useful in confirming that the main population is monomer and to characterize any higher order aggregates that may be present. In certain embodiments, the Z-average value of the pharmaceutical formulation does not increase by more than 5%, 4%, or 15% after incubation at 4° C. for 3 weeks. In certain embodiments, the Z-average value of the pharmaceutical formulation does not increase by more than 5%, 4%, 15%, 20%, or 25% after incubation at 50° C. for 3 weeks. In certain embodiments, the PDI value of the pharmaceutical formulation does not increase by more than 4%, 20%, 30%, 40%, or 50% after incubation at 4° C. for 3 weeks. In certain embodiments, the PDI value of the pharmaceutical formulation does not increase by more than 2-fold, 3-fold, 4-fold, or 5-fold after incubation at 50° C. for 3 weeks.

In some embodiments, the pharmaceutical formulation is stable at room temperature for at least 1 month, at least 2 months, at least 3 months, at least 4 months, at least 5 months, at least 6 months, at least 7 months, at least 8 months, at least 9 months, at least 10 months, at least 11 months, at least 1 year, at least 1.5 years, or at least 2 years. In some embodiments, the pharmaceutical formulation is stable at −80° C. for at least 1 month, at least 2 months, at least 3 months, at least 4 months, at least 5 months, at least 6 months, at least 7 months, at least 8 months, at least 9 months, at least 10 months, at least 11 months at least 1 year, at least 1.5 years, or at least 2 years. In some embodiments, the pharmaceutical formulation is stable at −20° C. for at least 1 month, at least 2 months, at least 3 months, at least 4 months, at least 5 months, at least 6 months, at least 7 months, at least 8 months, at least 9 months, at least 10 months, at least 11 months, at least 1 year, at least 1.5 years, or at least 2 years. In some embodiments, the pharmaceutical formulation is stable at −5° C. for at least 1 month, at least 2 months, at least 3 months, at least 4 months, at least 5 months, at least 6 months, at least 7 months, at least 8 months, at least 9 months, at least 10 months, at least 11 months, at least 1 year, at least 1.5 years, or at least 2 years. In some embodiments, the pharmaceutical formulation is stable at refrigerated temperatures for at least 1 month, at least 2 months, at least 3 months, at least 4 months, at least 5 months, at least 6 months, at least 7 months, at least 8 months, at least 9 months, at least 10 months, at least 11 months, at least 1 year, at least 1.5 years, or at least 2 years.

As used herein, “room temperatures” are temperatures refers to ambient temperatures as understood by one of ordinary skill in the art. For example, room temperatures can include 10-30° C. or 20-25° C. For example, room temperature includes, but is not limited to, 10-30° C., 15-30° C., 20-30° C., 25-30° C., 10-25° C., 15-25° C., 20-25° C., 10-20° C., 15-20° C., or 10-15° C.

As used herein, “refrigerated temperatures” are temperatures at or below 4° C. For example, refrigerated temperatures include, but are not limited to, 2 to 4° C., 1 to 4° C., 0 to 4° C., −2 to 4° C., −3 to 4° C., −4 to 4° C., or −5 to 4° C.

### Dosage Forms

The pharmaceutical formulation can be prepared and stored as a liquid formulation or a lyophilized form. In certain embodiments, the pharmaceutical formulation is a liquid formulation for storage at 2-8° C. (e.g., 4° C.) or a frozen formulation for storage at −20° C. or lower. The sugar or sugar alcohol in the formulation is used as a lyoprotectant.

Prior to pharmaceutical use, the pharmaceutical formulation can be diluted in an appropriate buffer or diluent. In some embodiments, the pharmaceutical formulation can be diluted in 15 mM to 25 mM citrate (e.g., 15 mM to 25 mM, 16 mM to 25 mM, 17 mM to 25 mM, 18 mM to 25 mM, 19 mM to 25 mM, 20 mM to 25 mM, 21 mM to 25 mM, 22 mM to 25 mM, 23 mM to 25 mM, 24 mM to 25 mM, 15 mM to 24 mM, 15 mM to 23 mM, 15 mM to 22 mM, 15 mM to 23 mM, 15 mM to 22 mM, 15 mM to 21 mM, 15 mM to 20 mM, 15 mM to 19 mM, 15 mM to 18 mM, 15 mM to 17 mM, 15 mM to 16 mM, or about 20 mM); 4% to 8% (w/v) mannitol (e.g., 4% to 8% (w/v), 4% to 7% (w/v), 4% to 6% (w/v), 4% to 5% (w/v), 5% to 8% (w/v), 6% to 8% (w/v), 7% to 8% (w/v), or about 6% (w/v)); and 0.005% to 0.05% (w/v) polysorbate 80 (e.g., 0.005% to 0.05% (w/v), 0.005% to 0.04% (w/v), 0.005% to 0.03% (w/v), 0.005% to 0.02% (w/v), 0.005% to 0.01% (w/v), 0.006% to 0.05% (w/v), 0.007% to 0.05% (w/v), 0.008% to 0.05% (w/v), 0.009% to 0.05% (w/v), 0.01% to 0.05% (w/v), 0.02% to 0.05% (w/v), 0.03% to 0.05% (w/v), 0.04% to 0.05% (w/v), or 0.01% (w/v)), at pH 6.2 to 6.8. In some embodiments, the pharmaceutical formulation can be diluted in 20 mM citrate; 6% (w/v) mannitol; and 0.01% (w/v) polysorbate 80, at pH 6.5. In some embodiments, the pharmaceutical formulation can be diluted with a suitable diluent in the range of 1:0, 1:1, 1:1.5, 1:2, 1:3, 1:4, 1:5, 1:6, 1:7, 1:8, 1:8, 1:10, 1:15, 1:20, 1:25, 1:50, or 1:100 prior to administration to a subject.

In alternative embodiments, the pharmaceutical formulation can be diluted and/or reconstituted in an aqueous carrier that is suitable for the route of administration. Other exemplary carriers include sterile water for injection (SWFI), bacteriostatic water for injection (BWFI), a pH buffered solution (e.g., phosphate-buffered saline), sterile saline solution, Ringer's solution, or dextrose solution. For example, when prepared for intravenous administration, the pharmaceutical formulation can be diluted in a 0.9% sodium chloride (NaCl) solution. In certain embodiments, the diluted pharmaceutical formulation is isotonic and suitable for administration by intravenous infusion.

The pharmaceutical formulation includes the multi-specific binding protein at a concentration suitable for storage. In certain embodiments, the pharmaceutical formulation includes the multi-specific binding protein at a concentration of 1 mg/ml to 200 mg/ml, 2 mg/ml to 200 mg/ml, 5 mg/ml to 200 mg/ml, 7.5 mg/ml to 200 mg/ml, 10 mg/ml to 200 mg/ml, 12.5 mg/ml to 200 mg/ml, 15 mg/ml to 200 mg/ml, 20 mg/ml to 200 mg/ml, 25 mg/ml to 200 mg/ml, 50 mg/ml to 200 mg/ml, 75 mg/ml to 200 mg/ml, 100 mg/ml to 200 mg/ml, 125 mg/ml to 200 mg/ml, 150 mg/ml to 200 mg/ml, 175 mg/ml to 200 mg/ml, 1 mg/ml to 150 mg/ml, 2 mg/ml to 150 mg/ml, 5 mg/ml to 150 mg/ml, 7.5 mg/ml to 150 mg/ml, 10 mg/ml to 150 mg/ml, 12.5 mg/ml to 150 mg/ml, 15 mg/ml to 150 mg/ml, 20 mg/ml to 150 mg/ml, 25 mg/ml to 150 mg/ml, 50 mg/ml to 150 mg/ml, 75 mg/ml to 150 mg/ml, 100 mg/ml to 150 mg/ml, 125 mg/ml to 150 mg/ml, 1 mg/ml to 100 mg/ml, 2 mg/ml to 100 mg/ml, 5 mg/ml to 100 mg/ml, 7.5 mg/ml to 100 mg/ml, 10 mg/ml to 100 mg/ml, 12.5 mg/ml to 100 mg/ml, 15 mg/ml to 100 mg/ml, 20 mg/ml to 100 mg/ml, 25 mg/ml to 100 mg/ml, 50 mg/ml to 100 mg/ml, 75 mg/ml to 100 mg/ml, 1 mg/ml to 50 mg/ml, 2 mg/ml to 50 mg/ml, 5 mg/ml to 50 mg/ml, 7.5 mg/ml to 50 mg/ml, 10 mg/ml to 50 mg/ml, 12.5 mg/ml to 50 mg/ml, 15 mg/ml to 50 mg/ml, 20 mg/ml to 50 mg/ml, 25 mg/ml to 50 mg/ml, 1 mg/ml to 25 mg/ml, 2 mg/ml to 25 mg/ml, 5 mg/ml to 25 mg/ml, 7.5 mg/ml to 25 mg/ml, 10 mg/ml to 25 mg/ml, 12.5 mg/ml to 25 mg/ml, 15 mg/ml to 25 mg/ml, 20 mg/ml to 25 mg/ml, 1 mg/ml to 20 mg/ml, 2 mg/ml to 20 mg/ml, 5 mg/ml to 20 mg/ml, 7.5 mg/ml to 20 mg/ml, 10 mg/ml to 20 mg/ml, 12.5 mg/ml to 20 mg/ml, or 15 mg/ml to 20 mg/ml.

In certain embodiments, the pharmaceutical formulation is packaged in a container (e.g., a vial, bag, pen, or syringe). In certain embodiments, the formulation may be a lyophilized formulation or a liquid formulation. In certain embodiments, the amount of multi-specific binding protein in the container is suitable for administration as a single dose. In certain embodiments, the amount of multi-specific binding protein in the container is suitable for administration in multiple doses. In certain embodiments, the pharmaceutical formulation includes the multi-specific binding protein at an amount of 0.1 to 2000 mg. In certain embodiments, the pharmaceutical formulation includes the multi-specific binding protein at an amount of 1 to 2000 mg, 10 to 2000 mg, 20 to 2000 mg, 50 to 2000 mg, 100 to 2000 mg, 200 to 2000 mg, 500 to 2000 mg, 1000 to 2000 mg, 0.1 to 1000 mg, 1 to 1000 mg, 10 to 1000 mg, 20 to 1000 mg, 50 to 1000 mg, 100 to 1000 mg, 200 to 1000 mg, 500 to 1000 mg, 0.1 to 500 mg, 1 to 500 mg, 10 to 500 mg, 20 to 500 mg, 50 to 500 mg, 100 to 500 mg, 200 to 500 mg, 0.1 to 200 mg, 1 to 200 mg, 10 to 200 mg, 20 to 200 mg, 50 to 200 mg, 100 to 200 mg, 0.1 to 100 mg, 1 to 100 mg, 10 to 100 mg, 20 to 100 mg, 50 to 100 mg, 0.1 to 50 mg, 1 to 50 mg, 10 to 50 mg, 20 to 50 mg, 0.1 to 20 mg, 1 to 20 mg, 10 to 20 mg, 0.1 to 10 mg, 1 to 10 mg, or 0.1 to 1 mg. In certain embodiments, the pharmaceutical formulation includes the multi-specific binding protein at an amount of 0.1 mg, 1 mg, 2 mg, 5 mg, 10 mg, 15 mg, 20 mg, 25 mg, 30 mg, 35 mg, 40 mg, 45 mg, 50 mg, 60 mg, 70 mg, 80 mg, 90 mg, 100 mg, 150 mg, 200 mg, 250 mg, 300 mg, 400 mg, 500 mg, 600 mg, 700 mg, 800 mg, 900 mg, 1000 mg, 1500 mg, or 2000 mg.

Actual dosage levels of the active ingredients in the pharmaceutical formulations described herein may be varied so as to obtain an amount of the active ingredient which is effective to achieve the desired therapeutic response for a particular patient, composition, and mode of administration, without being toxic to the patient.

The specific dose can be a uniform dose for each patient, for example, 1-5000 mg of protein. Alternatively, a patient's dose can be tailored to the approximate body weight or surface area of the patient. Other factors in determining the appropriate dosage can include the disease or condition to be treated or prevented, the severity of the disease, the route of administration, and the age, sex and medical condition of the patient. Further refinement of the calculations necessary to determine the appropriate dosage for treatment is routinely made by those skilled in the art, especially in light of the dosage information and assays disclosed herein. The dosage can also be determined through the use of known assays for determining dosages used in conjunction with appropriate dose-response data. An individual patient's dosage can be adjusted as the progress of the disease is monitored. Blood levels of the targetable construct or complex in a patient can be measured to see if the dosage needs to be adjusted to reach or maintain an effective concentration. Pharmacogenomics may be used to determine which targetable constructs and/or complexes, and dosages thereof, are most likely to be effective for a given individual (Schmitz et al., Clinica Chimica Acta 308: 43-53, 2001; Steimer et al., Clinica Chimica Acta 308: 33-41, 2001).

In general, dosages based on body weight are from about 0.1 mg/kg to 150 mg/mg, 0.5 mg/kg to 150 mg/kg, 1.0 mg/kg to 150 mg/kg, 1.5 mg/kg to 150 mg/kg, 2.0 mg/kg to 150 mg/kg, 2.5 mg/kg to 150 mg/kg, 5.0 mg/kg to 150 mg/kg, 7.5 mg/kg to 150 mg/kg, 10 mg/kg to 150 mg/kg, 15 mg/kg to 150 mg/kg, 20 mg/kg to 150 mg/kg, 25 mg/kg to 150 mg/kg, 50 mg/kg to 150 mg/kg, 75 mg/kg to 150 mg/kg, 100 mg/kg to 150 mg/kg, 125 mg/kg to 150 mg/kg, 0.1 mg/kg to 125 mg/mg, 0.5 mg/kg to 125 mg/kg, 1.0 mg/kg to 125 mg/kg, 1.5 mg/kg to 125 mg/kg, 2.0 mg/kg to 125 mg/kg, 2.5 mg/kg to 125 mg/kg, 5.0 mg/kg to 125 mg/kg, 7.5 mg/kg to 125 mg/kg, 10 mg/kg to 125 mg/kg, 15 mg/kg to 125 mg/kg, 20 mg/kg to 125 mg/kg, 25 mg/kg to 125 mg/kg, 50 mg/kg to 125 mg/kg, 75 mg/kg to 125 mg/kg, 100 mg/kg to 125 mg/kg, 0.1 mg/kg to 100 mg/mg, 0.5 mg/kg to 100 mg/kg, 1.0 mg/kg to 100 mg/kg, 1.5 mg/kg to 100 mg/kg, 2.0 mg/kg to 100 mg/kg, 2.5 mg/kg to 100 mg/kg, 5.0 mg/kg to 100 mg/kg, 7.5 mg/kg to 100 mg/kg, 10 mg/kg to 100 mg/kg, 15 mg/kg to 100 mg/kg, 20 mg/kg to 100 mg/kg, 25 mg/kg to 100 mg/kg, 50 mg/kg to 100 mg/kg, 75 mg/kg to 100 mg/kg, 0.1 mg/kg to 75 mg/mg, 0.5 mg/kg to 75 mg/kg, 1.0 mg/kg to 75 mg/kg, 1.5 mg/kg to 75 mg/kg, 2.0 mg/kg to 75 mg/kg, 2.5 mg/kg to 75 mg/kg, 5.0 mg/kg to 75 mg/kg, 7.5 mg/kg to 75 mg/kg, 10 mg/kg to 75 mg/kg, 15 mg/kg to 75 mg/kg, 20 mg/kg to 75 mg/kg, 25 mg/kg to 75 mg/kg, 50 mg/kg to 75 mg/kg, 0.1 mg/kg to 50 mg/mg, 0.5 mg/kg to 50 mg/kg, 1.0 mg/kg to 50 mg/kg, 1.5 mg/kg to 50 mg/kg, 2.0 mg/kg to 50 mg/kg, 2.5 mg/kg to 50 mg/kg, 5.0 mg/kg to 50 mg/kg, 7.5 mg/kg to 50 mg/kg, 10 mg/kg to 50 mg/kg, 15 mg/kg to 50 mg/kg, 20 mg/kg to 50 mg/kg, 25 mg/kg to 50 mg/kg, 0.1 mg/kg to 25 mg/mg, 0.5 mg/kg to 25 mg/kg, 1.0 mg/kg to 25 mg/kg, 1.5 mg/kg to 25 mg/kg, 2.0 mg/kg to 25 mg/kg, 2.5 mg/kg to 25 mg/kg, 5.0 mg/kg to 25 mg/kg, 7.5 mg/kg to 25 mg/kg, 10 mg/kg to 25 mg/kg, 15 mg/kg to 25 mg/kg, 20 mg/kg to 25 mg/kg, 0.1 mg/kg to 20 mg/mg, 0.5 mg/kg to 20 mg/kg, 1.0 mg/kg to 20 mg/kg, 1.5 mg/kg to 20 mg/kg, 2.0 mg/kg to 20 mg/kg, 2.5 mg/kg to 20 mg/kg, 5.0 mg/kg to 20 mg/kg, 7.5 mg/kg to 20 mg/kg, 10 mg/kg to 20 mg/kg, or 15 mg/kg to 20 mg/kg.

For example, dosages based on body weight can be from about 5 mg/kg to 50 mg/kg (5 mg/kg to 50 mg/kg, 5 mg/kg to 45 mg/kg, 5 mg/kg to 40 mg/kg, 5 mg/kg to 35 mg/kg, 5 mg/kg to 30 mg/kg, 5 mg/kg to 25 mg/kg, 5 mg/kg to 20 mg/kg, 5 mg/kg to 15 mg/kg, 5 mg/kg to 10 mg/kg, 10 mg/kg to 50 mg/kg, 10 mg/kg to 45 mg/kg, 10 mg/kg to 40 mg/kg, 10 mg/kg to 35 mg/kg, 10 mg/kg to 30 mg/kg, 10 mg/kg to 25 mg/kg, 10 mg/kg to 20 mg/kg, 10 mg/kg to 15 mg/kg, 15 mg/kg to 50 mg/kg, 15 mg/kg to 45 mg/kg, 15 mg/kg to 40 mg/kg, 15 mg/kg to 35 mg/kg, 15 mg/kg to 30 mg/kg, 15 mg/kg to 25 mg/kg, 15 mg/kg to 20 mg/kg, 20 mg/kg to 50 mg/kg, 20 mg/kg to 45 mg/kg, 20 mg/kg to 40 mg/kg, 20 mg/kg to 35 mg/kg, 20 mg/kg to 30 mg/kg, 20 mg/kg to 25 mg/kg, 25 mg/kg to 50 mg/kg, 25 mg/kg to 45 mg/kg, 25 mg/kg to 40 mg/kg, 25 mg/kg to 35 mg/kg, 25 mg/kg to 30 mg/kg, 30 mg/kg to 50 mg/kg, 30 mg/kg to 45 mg/kg, 30 mg/kg to 40 mg/kg, 30 mg/kg to 35 mg/kg, 35 mg/kg to 50 mg/kg, 35 mg/kg to 45 mg/kg, 35 mg/kg to 40 mg/kg, 40 mg/kg to 50 mg/kg, 40 mg/kg to 45 mg/kg, or 45 mg/kg to 50 mg/kg). In some embodiments, the pharmaceutical formulation is administered at a dose of 0.1 mg/kg.

In some embodiments, the pharmaceutical formulation is administered on day 1 of a first cycle at a priming dose from about 1 mg/kg to about 10 mg/kg (1 mg/kg to 10 mg/kg, 1 mg/kg to 9 mg/kg, 1 mg/kg to 8 mg/kg, 1 mg/kg to 7 mg/kg, 1 mg/kg to 6 mg/kg, 1 mg/kg to 5 mg/kg, 1 mg/kg to 4 mg/kg, 1 mg/kg to 3 mg/kg, 1 mg/kg to 2 mg/kg, 2 mg/kg to 10 mg/kg, 2 mg/kg to 9 mg/kg, 2 mg/kg to 8 mg/kg, 2 mg/kg to 7 mg/kg, 2 mg/kg to 6 mg/kg, 2 mg/kg to 5 mg/kg, 2 mg/kg to 4 mg/kg, 2 mg/kg to 3 mg/kg, 3 mg/kg to 10 mg/kg, 3 mg/kg to 9 mg/kg, 3 mg/kg to 8 mg/kg, 3 mg/kg to 7 mg/kg, 3 mg/kg to 6 mg/kg, 3 mg/kg to 5 mg/kg, 3 mg/kg to 4 mg/kg, 4 mg/kg to 10 mg/kg, 4 mg/kg to 9 mg/kg, 4 mg/kg to 8 mg/kg, 4 mg/kg to 7 mg/kg, 4 mg/kg to 6 mg/kg, 4 mg/kg to 5 mg/kg, 5 mg/kg to 10 mg/kg, 5 mg/kg to 9 mg/kg, 5 mg/kg to 8 mg/kg, 5 mg/kg to 7 mg/kg, 5 mg/kg to 6 mg/kg, 6 mg/kg to 10 mg/kg, 6 mg/kg to 9 mg/kg, 6 mg/kg to 8 mg/kg, 6 mg/kg to 7 mg/kg, 7 mg/kg to 10 mg/kg, 7 mg/kg to 9 mg/kg, 7 mg/kg to 8 mg/kg, 8 mg/kg to 10 mg/kg, 8 mg/kg to 9 mg/kg, or 9 mg/kg to 10 mg/kg). For example, in some embodiments, the pharmaceutical formulation is administered on day 1 of a first cycle at a priming dose of about 3 mg/kg.

In some embodiments, the pharmaceutical formulation may be administered once daily, weekly, monthly, or yearly. In some embodiments, the pharmaceutical formulation is administered once weekly in one or more 4-week treatment cycles. In some embodiments, the pharmaceutical formulation is administered to the subject on day 1, day 8, day 15, and day 22 of the one or more 4-week treatment cycles. In some embodiments, a subject may receive 1, 2, 3, 4, 5, 6, 7, 8, 9, 10, or more 4-week treatment cycles. In some embodiments, after a completed 4-week treatment cycle, the pharmaceutical formulation is administered to the subject to achieve an increased dose of the multi-specific binding protein in a subsequent 4-week treatment cycle as compared to the earlier completed 4-week treatment cycle.

In some embodiments, pharmaceutical formulations of the present disclosure are administered as a monotherapy. In some embodiments, pharmaceutical formulations of the present disclosure are administered in combination with other anti-cancer therapies (e.g., anti-PD-1 and/or anti-PD-L1 therapies). In some embodiments, the pharmaceutical formulation is administered concurrently with the anti-PD-1 and/or anti-PD-L1 therapy. In some embodiments, the pharmaceutical formulation is administered in combination with nivolumab. In some embodiments, nivolumab is administered on day 1, day 8, day 15, or day 22, of a 4-week treatment cycle. In some embodiments, the pharmaceutical formulation is administered in combination with pembrolizumab. In some embodiments, pembrolizumab is administered every 6 weeks.

Persons of ordinary skill in the art can easily estimate repetition rates for dosing based on measured residence times and concentrations of the targetable construct or complex in bodily fluids or tissues. Administration of the pharmaceutical formulations described herein can be intravenous, intraarterial, intraperitoneal, intramuscular, subcutaneous, intrapleural, intrathecal, intracavitary, by perfusion through a catheter or by direct intralesional injection.

The description above describes multiple aspects and embodiments of the present application. The patent application specifically contemplates all combinations and permutations of the aspects and embodiments.

### VII. Method of Purification

The present disclosure also provides methods of purifying a multi-specific protein disclosed as described herein.

In some embodiments, a purification method includes one or more steps selected from: a protein A affinity purification; a low pH viral inactivation; a mix-mode anion exchange chromatography; a mixed-mode chromatography; a viral filtration; and ultrafiltration/diafiltration.

In some embodiments, protein A affinity purification includes: (a) binding the multi-specific binding protein to a protein A resin; and (b) eluting the bound multi-specific binding protein at a pH of 3.5-3.8, thereby producing a first eluate. In some embodiments, the bound multi-specific binding protein is eluted at a pH of about 3.7. In some embodiments, the pH of the first eluate is 4.0-4.6. In some embodiments the pH of the first eluate is about 4.3.

In some embodiments, the low pH viral inactivation step includes: addition of an amount of acetic acid to the first eluate sufficient to achieve a pH of 3.5-3.7, thereby producing the viral inactivation reaction mixture; and incubation of the viral inactivation reaction mixture for 30-60 minutes. In some embodiments, the low pH viral inactivation reaction mixture is about 3.6. In some embodiments the purification method includes addition of an amount of Tris sufficient to bring the pH of the low pH viral inactivation reaction mixture to a pH of 6.3-6.5 following the incubation.

In some embodiments, the purification method includes mix-mode anion exchange chromatography including flowing the viral inactivation reaction mixture through an anion exchange column, thereby producing a second eluate.

In some embodiments, the purification method includes a mixed-mode chromatography step including: flowing the second eluate through a ceramic hydroxyapatite (CHT), type I column; and eluting the multi-specific binding protein from the CHT column in a buffer including about 200 mM sodium chloride, thereby producing a third eluate.

In some embodiments, the method further includes a buffer exchange step of the multi-specific binding protein to achieve a final buffer concentration of 15 mM to 25 mM citrate (e.g., 15 mM to 25 mM, 16 mM to 25 mM, 17 mM to 25 mM, 18 mM to 25 mM, 19 mM to 25 mM, 20 mM to 25 mM, 21 mM to 25 mM, 22 mM to 25 mM, 23 mM to 25 mM, 24 mM to 25 mM, 15 mM to 24 mM, 15 mM to 23 mM, 15 mM to 22 mM, 15 mM to 23 mM, 15 mM to 22 mM, 15 mM to 21 mM, 15 mM to 20 mM, 15 mM to 19 mM, 15 mM to 18 mM, 15 mM to 17 mM, 15 mM to 16 mM, or about 20 mM), 4% to 8% (w/v) mannitol (e.g., 4% to 8% (w/v), 4% to 7% (w/v), 4% to 6% (w/v), 4% to 5% (w/v), 5% to 8% (w/v), 6% to 8% (w/v), 7% to 8% (w/v), or about 6% (w/v)), and 0.005% to 0.05% (w/v) polysorbate 80 (e.g., 0.005% to 0.05% (w/v), 0.005% to 0.04% (w/v), 0.005% to 0.03% (w/v), 0.005% to 0.02% (w/v), 0.005% to 0.01% (w/v), 0.006% to 0.05% (w/v), 0.007% to 0.05% (w/v), 0.008% to 0.05% (w/v), 0.009% to 0.05% (w/v), 0.01% to 0.05% (w/v), 0.02% to 0.05% (w/v), 0.03% to 0.05% (w/v), 0.04% to 0.05% (w/v), or 0.01% (w/v)), at pH 6.2 to 6.8 (e.g., 6.2 to 6.8, 6.2 to 6.7, 6.2 to 6.6, 6.2 to 6.5, 6.2 to 6.4, 6.2 to 6.3, 6.3 to 6.8, 6.4 to 6.8, 6.5 to 6.8, 6.6 to 6.8, 6.7 to 6.8, or about 6.5).

### EXAMPLES

The following examples are merely illustrative and are not intended to limit the scope or content of the multi-specific binding proteins described in the present application in any way.

### Example 1: Preparation of EGFR-TriNKET

### Description and Composition of the Drug Product

An exemplary pharmaceutical formulation of EGFR-TriNKET, incorporating: (a) a first polypeptide with the amino acid sequence of SEQ ID NO:167; (b) a second polypeptide with the amino acid sequence of SEQ ID NO:164; and (c) a third polypeptide with the amino acid sequence of SEQ ID NO:165, was prepared as a sterile solution with 20-mM citrate, 6% mannitol (w/v), 0.01% polysorbate 80 (w/v), at pH 6.5 (See Table 12). The formulation listed in Table 12 includes diluted drug substance (EGFR-TriNKET) and buffer and excipients, and was prepared for filling into drug product vials, without further addition of excipients or exchange of formulation buffer.

TABLE 12
Exemplary Pharmaceutical Formulation
of EGFR-TriNKET Drug Product

| Component | Amount | |
|---|---|---|
| EGFR-TriNKET | 15 | mg/mL |
| Trisodium citrate, dihydrate | 5.55 | mg/mL |
| Citric acid, monohydrate | 0.23 | mg/mL |
| Mannitol | 60 | mg/mL |

| | |
|---|---|
| | (6% w/v) |
| Polysorbate 80 | 0.01% (w/v) |

### Drug Product Manufacturing Process

The frozen drug substance was transported to ThermoFisher (Patheon) (Greenville, N.C., USA) and was stored at nominal −70° C. until use. At the start of drug product manufacturing, the drug substance was thawed and individual containers were pooled. The pool was mixed, and formulation buffer (20 mM citrate, 6% mannitol [w/v], 0.01% polysorbate 80 [w/v], pH 6.5) was added to dilute the pool to the final drug product concentration. The diluted mixture was mixed, sterile-filtered through a 0.2-μm membrane, and was filled into 20-cc Type I borosilicate glass vials with 20-mm stoppers and seals.

### Detection of Product-Related Impurities

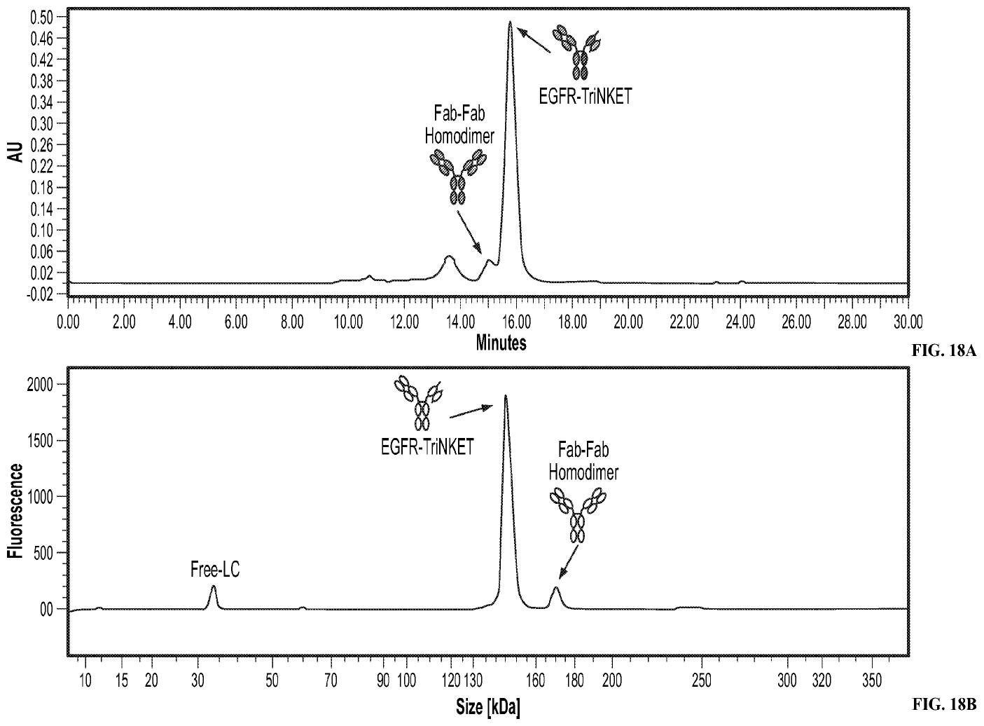

For detection of impurities, analytical methods capable of resolving the main EGFR-TriNKET product from product-related impurities that may appear in the drug substance or drug product were developed. These methods were used to monitor product-related species during production, as well as during product storage. There were 2 main methods used for monitoring product-related species: size-exclusion chromatography (SEC) and capillary gel electrophoresis with sodium dodecyl sulfate (CE-SDS) under non-reducing conditions. SEC was used to quantify high molecular weight species (e.g., protein aggregates) that may be present, while CE-SDS was used to quantify both high molecular weight species (e.g., Fab-Fab homodimer) or low molecular weight species (e.g., protein fragments) that may be present. Examples of a representative SEC-HPLC chromatogram and CE-SDS electropherogram under non-reducing conditions are shown in FIG. 18A (an SEC-HPLC chromatogram) and FIG. 18B (CE-SDS (Non-Reducing) electropherogram).

### Example 2: Characterization of EGFR-TriNKET

EGFR-TriNKET is a heterodimeric tri-specific antibody engineered to bind to both EGFR and NKG2D, while retaining a fully functional human IgG1 Fc domain capable of also binding to Fc receptors.

### EGFR-Binding Characteristics of EGFR-TriNKET

EGFR-TriNKET, incorporating: (a) a first polypeptide with the amino acid sequence of SEQ ID NO:167; (b) a second polypeptide with the amino acid sequence of SEQ ID NO:164; and (c) a third polypeptide with the amino acid sequence of SEQ ID NO:165, was designed to direct immune cell activity against tumors via a high-affinity, EGFR-targeting arm. The EGFR-binding characteristics of EGFR-TriNKET were evaluated by both surface plasmon resonance (SPR) using recombinant human EGFR and by flow cytometry using EGFR-expressing human tumor cell lines.

The binding kinetics and affinity of EGFR-TriNKET for purified recombinant human EGFR were obtained using SPR. Measurements were performed using a Biacore8K SPR instrument. Commercially available cetuximab and panitumumab were used as EGFR-binding controls. As presented in Table 13, EGFR-TriNKET bound human EGFR with an equilibrium dissociation constant (K_{D}) of 4.7 nM, while cetuximab and panitumumab bound the target with a K_{D} of 4.0 and 1.0 nM, respectively. The affinity of EGFR-TriNKET for EGFR was comparable to that of cetuximab and 4.7-fold weaker than that of panitumumab. The derived values for affinity and kinetic rates of cetuximab and panitumumab binding EGFR were in line with previously reported ones (panitumumab BLA 125147/0, cetuximab BLA 125084/0), with panitumumab having higher overall affinity, with considerably slower off rate and somewhat lower on rate.

TABLE 13
Kinetics and Affinity of EGFR-TriNKET Binding
to Human EGFR Evaluated by SPR at 37° C.

| Test Article | kₐ (1/Ms) | kₐ (1/s) | K_{D} (nM) |
|---|---|---|---|
| EGFR-TriNKET | (2.0 ± 0.0) × 10⁵ | (9.5 ± 0.0) × 10⁻⁴ | 4.7 ± 0.1 |
| Cetuximab | (1.4 ± 0.0) × 10⁶ | (5.6 ± 0.1) × 10⁻³ | 4.0 ± 0.2 |
| Panitumumab | (6.2 ± 0.2) × 10⁵ | (6.1 ± 0.2) × 10⁻⁴ | 1.0 ± 0.0 |

A panel of human tumor cell lines that endogenously express EGFR was characterized for cell surface expression of EGFR using fluorescent quantitation of directly labeled EGFR-TriNKET. The tumor cell lines tested exhibited surface expression of EGFR ranging from 1,000 to >1,000,000 average molecules of cell surface EGFR per cell.

The dose-responsive binding profile of EGFR-TriNKET to EGFR-expressing cell lines Detroit 562 (pharyngeal carcinoma), NCI-H1703 (non-small-cell lung cancer [NSCLC], squamous cell), and HT29 (colorectal adenocarcinoma) was compared to that of cetuximab and panitumumab. Test articles were dose titrated on EGFR-expressing cells and detected via flow cytometry using an anti-human Fc secondary antibody conjugated with fluorophore phycoerythrin (PE). As shown in FIG. 19A (binding to Detroit 562 tumor cells), FIG. 19B (binding to NCI-H1703 tumor cells), and FIG. 19C (binding to HT29 tumor cells), EGFR-TriNKET demonstrated high-affinity binding to all EGFR-expressing cell lines, with concentrations resulting in a half-maximal response (EC₅₀) ranging from 0.33 to 11 nM (Table 14). EGFR-TriNKET binding EC₅₀ showed some correlation with the level of EGFR expression (FIG. 19A (binding to Detroit 562 tumor cells), FIG. 19B (binding to NCI-H1703 tumor cells), and FIG. 19C (binding to HT29 tumor cells)). Overall, in these experiments, EGFR-TriNKET was able to bind cells with a slightly reduced affinity compared to that of cetuximab and panitumumab, which had binding EC₅₀ values ranging from 0.077 to 5.7 nM (Table 14). Without being bound to the theory, the weaker binding of EGFR-TriNKET may be assumed to be attributed to: (1) the scFv format of the EGFR-binding domain of EGFR-TriNKET rather than the antigen-binding fragment (Fab) arm format of mAbs, and/or (2) the single-arm/monovalent binding of EGFR-TriNKET compared to the bivalent nature of cetuximab and panitumumab. Monovalent binding of EGFR-TriNKET may also account for the higher maximum levels of EGFR-TriNKET binding on the cell lines compared to that seen with cetuximab and panitumumab, allowing for a EGFR-TriNKET-to-EGFR binding ratio close to 1:1.

TABLE 14
Binding Affinity of EGFR-TriNKET and EGFR Antibodies on EGFR-Expressing Human Tumor Cell Lines

| | | | EGFR-TriNKET | | Cetuximab | | Panitumumab | |
|---|---|---|---|---|---|---|---|---|
| | | Average EGFR | EC₅₀ | Max | EC₅₀ | Max | EC₅₀ | Max |
| Cell Line | Origin; Details | Per Cell | (nM) | FOB | (nM) | FOB | (nM) | FOB |
| KYSE-30 | EsoSCC; EGFRwt | >1,000,000 | 11 | 1800 | 1.6 | 1100 | — | — |
| FaDu | HNSCC; EGFRwt | 590,000 | 11 | 1200 | 5.7 | 820 | — | — |
| Detroit 562 | HNSCC; EGFRwt | 440,000 | 1.6 | 490 | 0.54 | 330 | 0.87 | 400 |
| NCI-H292 | NSCLC; EGFRwt | 400,000 | 5.3 | 800 | 1.0 | 590 | — | — |
| NCI-H1703 | NSCLC-squam; EGFRwt | 350,000 | 4.6 | 730 | 0.80 | 500 | 1.2 | 610 |
| 786-O | RCC; EGFRwt | 320,000 | 3.5 | 330 | 0.86 | 270 | — | — |
| NCI-H1975 | NSCLC-adeno; EGFRmut | 250,000 | 2.3 | 170 | 0.21 | 130 | — | — |
| | T790M, L858R | | | | | | | |
| HCC4006 | NSCLC-adeno; EGFRmut | 140,000 | 1.8 | 160 | 0.42 | 120 | 0.46 | 130 |
| | L747-E749del, A750P | | | | | | | |
| HCT116 | CRC; EGFRwt, KRASmut | 88,000 | 1.4 | 300 | 0.068 | 220 | — | — |
| | G13D | | | | | | | |
| HT29 | CRC; EGFRwt, KRASwt | 85,000 | 1.3 | 340 | 0.28 | 260 | 0.28 | 300 |
| NCI-H2172 | NSCLC; EGFRwt | 48,000 | 1.0 | 140 | 0.34 | 100 | 0.13 | 140 |
| BT474 | BRCA; EGFRwt | 20,000 | 0.77 | 41 | 0.089 | 30 | 0.35 | 42 |
| MDA-MB- | BRCA; EGFRwt, KRASmut | 1000 | 0.33 | 5.0 | PF | PF | 0.077 | 6.0 |
| 453 | G13D | | | | | | | |

### Fc Receptor and NKG2D Binding Characteristics of EGFR-TriNKET

To assess binding of EGFR-TriNKET to human FcR and NKG2D, SPR was performed using purified recombinant human Fc receptors and NKG2D, and flow cytometry was performed using human NK cell lines and human peripheral blood mononuclear cells (PBMCs).

Using SPR (Biacore 8K instrument), binding kinetics and affinity of EGFR-TriNKET for purified recombinant human Fc gamma receptors (FcRs) and human NKG2D were evaluated. Commercially available EGFR-targeting IgGs cetuximab (IgG1) and panitumumab (IgG2) were included in the experiment for comparison, and another marketed biologic (trastuzumab) was used as an experimental IgG(control. The data provided in Table 15 demonstrate that EGFR-TriNKET was able to find to human FcγRs with a K_{D} comparable to those of control IgG1s, while IgG2 panitumumab exhibited a different binding profile. EGFR-TriNKET and the therapeutic biologics tested in this experiment demonstrated comparable binding to human FcRn at pH 6.0, and lacked quantifiable binding to FcRn at pH 7.4. In addition, in these experiments, EGFR-TriNKET was able to bind to human NKG2D with a K_{D} of 481.5 nM (Table 16).

TABLE 15
Kinetics and Affinity of EGFR-TriNKET Binding
to Human FcRs Evaluated by SPR at 25° C.

| | | | |
|---|---|---|---|
| Receptor | ka (1/Ms) | kd (1/s) | Kd (M) |

FcγRI

| EGFR- | (4.2 ± 0.1) × 10⁴ | (2.4 ± 0.0) × 10⁻⁴ | (5.6 ± 0.1) × 10⁻⁹ |
|---|---|---|---|
| TriNKET | | | |
| Cetuximab | (3.2 ± 0.0) × 10⁴ | (2.3 ± 0.0) × 10⁻⁴ | (7.3 ± 0.1) × 10⁻⁹ |
| Panitumumab | No quantifiable binding at the concentrations tested | | |
| Trastuzumab | (8.2 ± 0.1) × 10⁴ | (2.4 ± 0.0) × 10⁻⁴ | (2.9 ± 0.1) × 10⁻⁹ |

FcγRIIa H131

| | | |
|---|---|---|
| EGFR- | NA | (1.0 ± 0.1) ×10⁻⁶ |
| TriNKET | | |
| Cetuximab | NA | (1.8 ± 0.2) × 10⁻⁶ |
| Panitumumab | NA | (1.1 ± 0.1) × 10⁻⁶ |
| Trastuzumab | NA | (1.2 ± 0.1) × 10⁻⁶ |

FcγRIIIa V158

| EGFR- | (2.0 ± 0.0) × 10⁵ | (1.1 ± 0.0) × 10⁻² | (53.8 ± 2.0) × 10⁻⁹ |
|---|---|---|---|
| TriNKET | | | |
| Cetuximab | (1.3 ± 0.0) × 10⁵ | (1.6 ± 0.0) × 10⁻² | (125.0 ± 2.3) × 10⁻⁹ |
| Panitumumab | No quantifiable binding at the concentrations tested | | |
| Trastuzumab | (2.1 ± 0.0) × 10⁵ | (1.0 ± 0.0) × 10⁻² | (48.2 ± 1.4) × 10⁻⁹ |

FcRn at pH 6.0

| | | |
|---|---|---|
| EGFR- | NA | (1.0 ± 0.1) × 10⁻⁶ |
| TriNKET | | |
| Cetuximab | NA | (1.8 ± 0.3) × 10⁻⁶ |
| Panitumumab | NA | (1.3 ± 0.3) × 10⁻⁶ |
| Trastuzumab | NA | (1.0 ± 0.1) × 10⁻⁶ |

FcRn at pH 7.4

| | |
|---|---|
| EGFR- | No quantifiable binding at the concentrations tested |
| TriNKET | |
| Cetuximab | |
| Panitumumab | |
| Trastuzumab | |

TABLE 16
Kinetics and Affinity of EGFR-TriNKET Binding
to Human NKG2D Evaluated by SPR at 25° C.

| Receptor | Test Article | kₐ (1/Ms) | k_{d} (1/s) | K_{D} (M) |
|---|---|---|---|---|
| NKG2D | EGFR-TriNKET | (2.3 ± 0.0) × 10⁵ | (1.1 ± 0.0) × 10⁻¹ | (481.5 ± 1.5) × 10⁻⁹ |

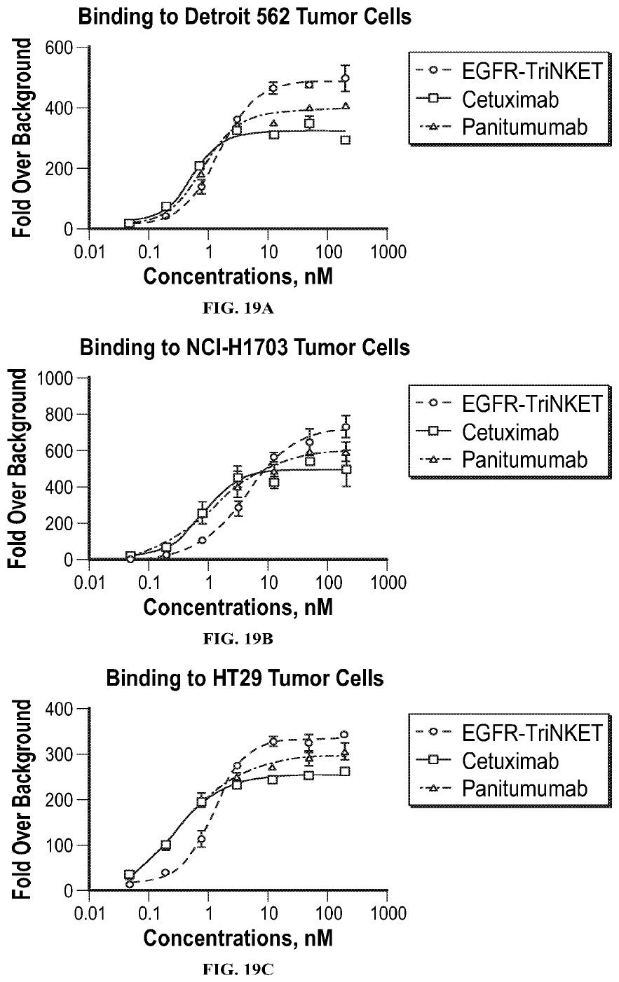

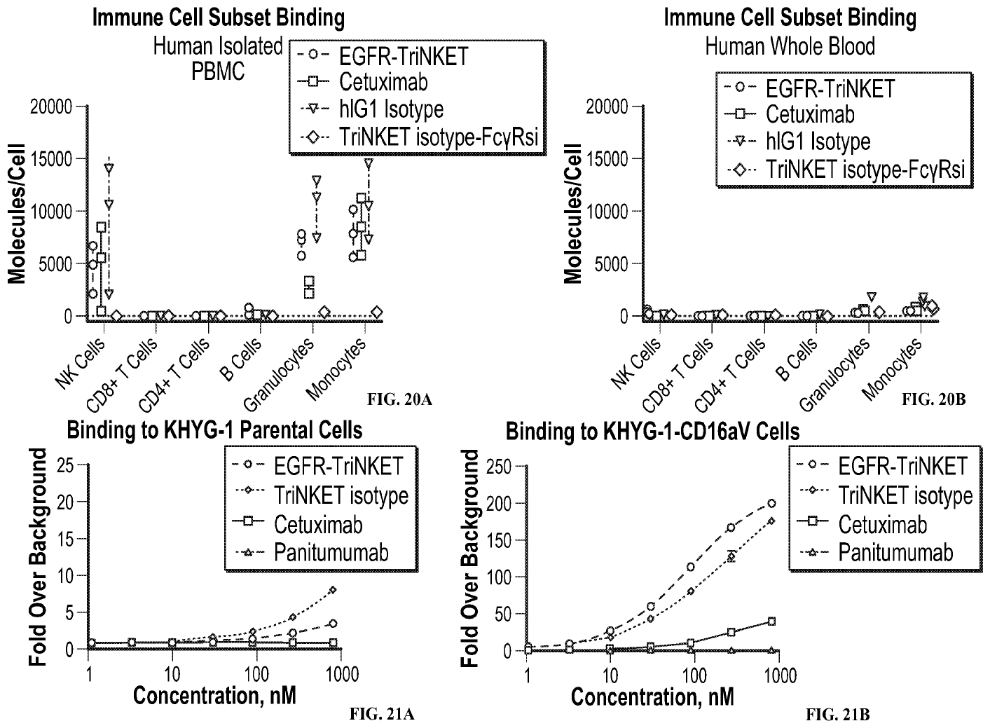

In further experiments, binding profile of EGFR-TriNKET to NK cells and other immune cell subsets was assessed by flow cytometry in isolated human PBMCs and whole blood samples using fluorophore-conjugated EGFR-TriNKET. Test articles, which were previously directly conjugated with Alexa Fluofrm 647 (AF647) for detection by flow cytometry, were introduced at 450 nM. The amount of each molecule bound per cell was calculated by comparing sample median fluorescence intensity signals to those of calibration beads coated with known amounts of AF647 and correcting for the AF647 degree of labeling for each test article. In these experiments, binding of EGFR-TriNKET was not detectable on NKG2D⁺ CD8⁺ T cells (FIG. 20A (immunce cell subset binding using human isolated PBMC) and FIG. 20B (immunce cell subset binding using human whole blood)). In these experiments, in addition to NK cells, binding of EGFR-TriNKET was also detected on monocytes and granulocytes (monocytes and granulocytes are NKG2D⁻ and FcγR⁺). In these experiments, cetuximab and a human IgG1 isotype control antibody demonstrated similar profiles of binding to FcγR⁺ cells as that of EGFR-TriNKET, while an isotype control TriNKET bearing mutations to abrogate FcγR engagement (TriNKET isotype-FcγRsi) did not bind detectably above background for any cell subset. In whole blood, EGFR-TriNKET demonstrated minimal binding to the same subsets; suggesting that, in these experiments, serum IgG might compete with EGFR-TriNKET for binding to FcγR. In these experiments, binding profiles of cetuximab, a human IgG1 isotype control, and TriNKET isotype-FcγRsi in whole blood were similar to that of EGFR-TriNKET. Taken together, the data presented in FIG. 20A (immunce cell subset binding using human isolated PBMC) and FIG. 20B (immunce cell subset binding using human whole blood) suggest a minimal immune cell binding profile in peripheral blood for EGFR-TriNKET, similar to that of an IgG1 mAb.

Experiments were also performed to assess binding properties of EGFR-TriNKET to NK cell lines, e.g., KHYG-1 parental cells and KHYG-1-CD16aV cells. In these experiments, binding of EGFR-TriNKET to NK cells was observed using NK cell lines (FIG. 21A (KHYG-1 parental cells) and FIG. 21B (KHYG-1-CD16aV cells)). Dose-responsive binding of EGFR-TriNKET, a TriNKET isotype control, cetuximab, and panitumumab, to the parental human NK cell line KHYG-1, which does not express CD16a, and on KHYG-1 cells transduced to express the high affinity 158V variant of CD16a (KHYG-1-CD16aV), were assessed. Test articles, which were previously directly conjugated with Alexa Fluor™ 647 for detection by flow cytometry, were dose titrated from 800 nM in 1:3 dilutions. In these experiments, EGFR-TriNKET bound with low affinity to parental KHYG-1 cells, which express NKG2D but not CD16a, with an EC₅₀>600 nM. On KHYG-1-CD16aV cells, which were transduced to stably express CD16a in addition to endogenously expressed NKG2D, EGFR-TriNKET bound with significantly greater magnitude compared to that of EGFR-TriNKET on KHYG-1 parental cells (p<0.0001). There was no significant binding of cetuximab to KHYG-1 parental cells (p=0.9324), which was significantly lower than that of EGFR-TriNKET on KHYG-1-CD16aV cells (p<0.0001). An isotype control TriNKET also showed low binding to parental KHYG-1 cells, but higher binding to KHYG-1-CD16aV cells to a similar extent as EGFR-TriNKET, suggesting that co-engagement of CD16a and NKG2D by EGFR-TriNKET results in a stronger binding profile to NK cells compared to that of cetuximab. In comparison with primary NK cells within PBMCs, the binding of EGFR-TriNKET may be more readily differentiated from that of cetuximab with KHYG-1-CD16aV because of their higher surface expression of NKG2D and CD16a. NKG2D and CD16a were individually present on KHYG-1-CD16aV cells in substantial excess of the total molecule/cell loading observed on primary NK cells (FIG. 21A (KHYG-1 parental cells) and FIG. 21B (KHYG-1-CD16aV cells)). Additionally, the ratio of NKG2D to CD16a on the surface of KHYG-1-CD16aV cells is higher than that on primary NK cells, which could further increase the ability to resolve the contribution of NKG2D to cell binding. Panitumumab, which contains an IgG2 Fc domain that is unable to bind to CD16a, bound negligibly to either NK cell line.

### EGFR-TriNKET Inhibits EGFR Signaling and Tumor Cell Growth

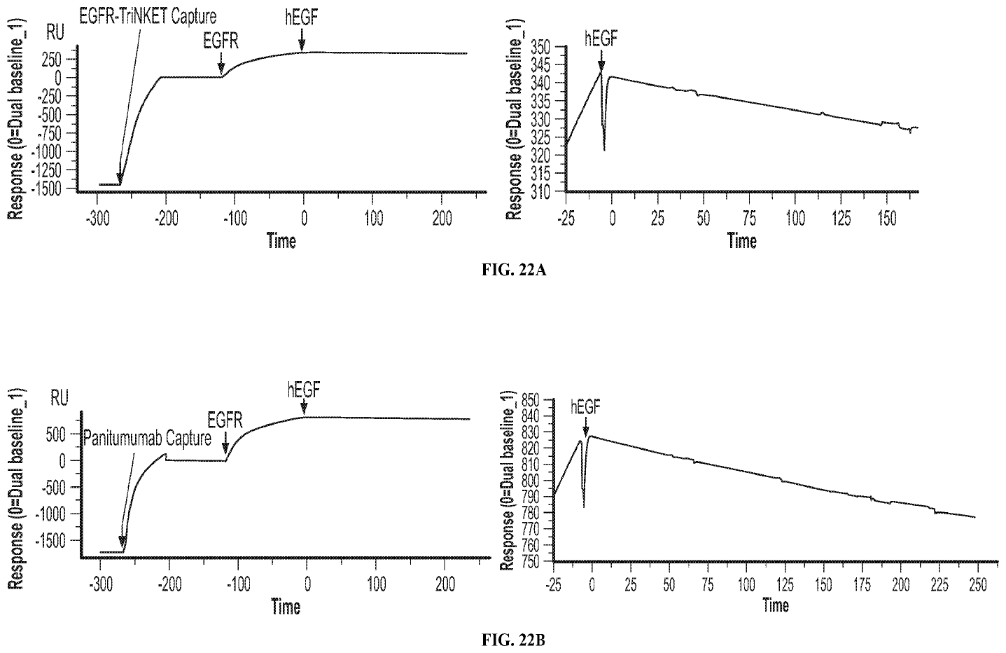

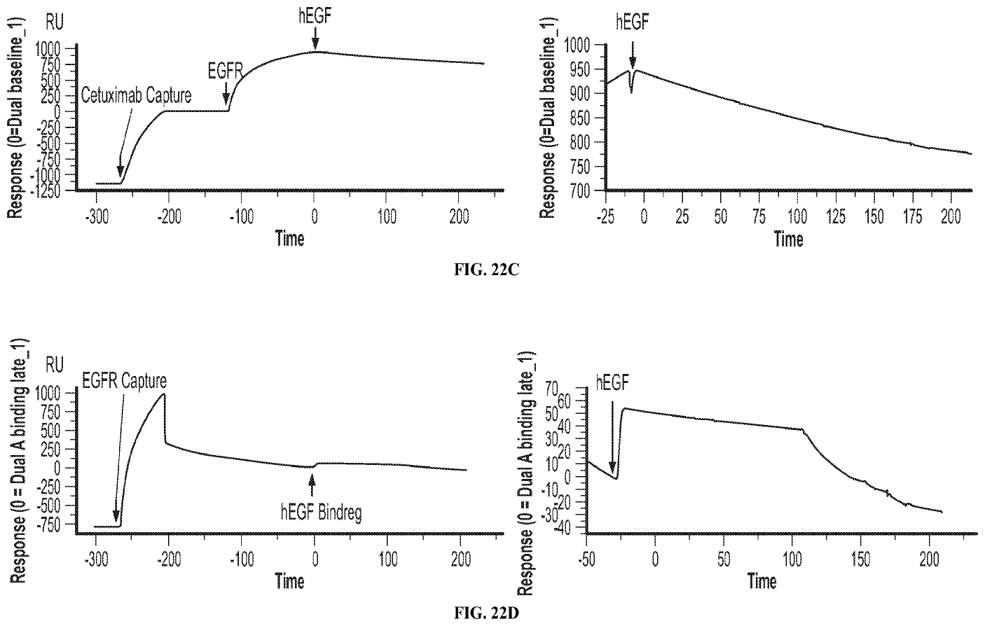

Inhibition of EGFR signaling through blockade of ligand binding is the major mechanism of action of cetuximab and panitumumab. As EGFR-TriNKET binds to EGFR with an scFv having variant sequences derived from the sequences of panitumumab (e.g., the sequence of VH or VL of an EGFR-TriNKET is a modified or variant sequence of the VH or VL of panitumumab), whether EGFR-TriNKET is capable of blocking ligand binding was investigated. Ligand blocking was assessed by SPR by injecting 400 nM of recombinant human EGF over recombinant EGFR that was first bound to captured EGFR-TriNKET, panitumumab, or cetuximab. Binding of 400 nM EGF to free EGFR captured via His-Tag served as a positive control. The SPR experiment presented in FIGS. 22A-22D confirms that EGFR-TriNKET, as well as panitumumab and cetuximab, indeed block binding of epidermal growth factor (EGF) to EGFR, while free receptor did bind recombinant human EGF (FIG. 22A shows ligand blocking of EGF to captured EGFR-TriNKET; FIG. 22B shows ligand blocking of EGF to captured panitumumab; FIG. 22C shows ligand blocking of EGF to captured cetuximab; FIG. 22D shows binding of EGF to free EGFR captured via His-Tag). Similar results were obtained for transforming growth factor alpha (TGF-α). Therefore, in addition to being a potent immune engager, EGFR-TriNKET has the capacity to inhibit wild-type EGFR signaling and downstream events.

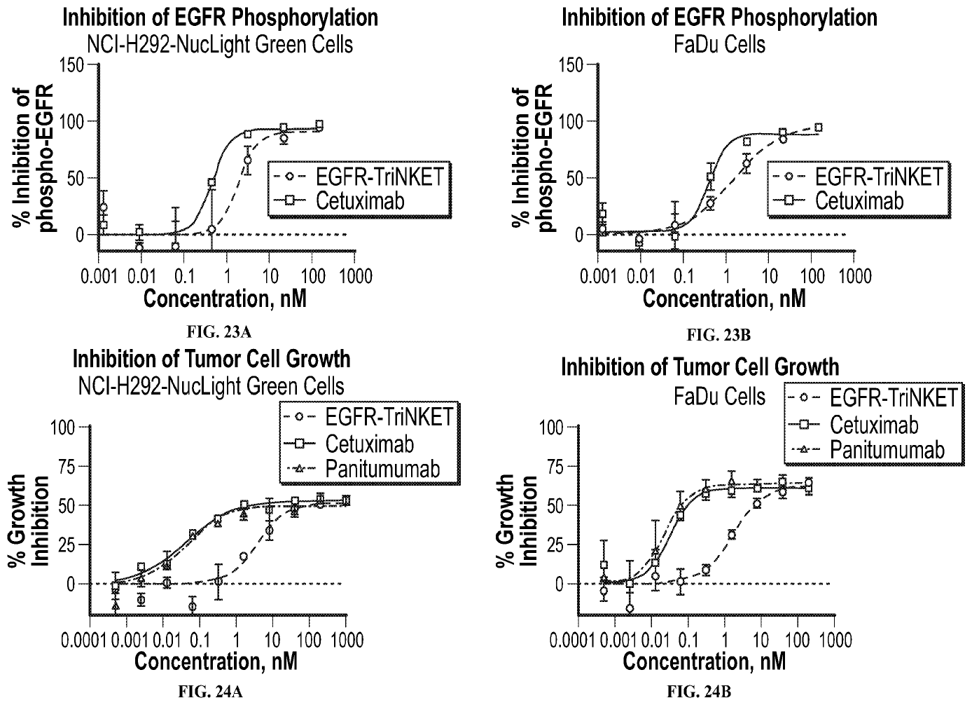

The EGFR signal inhibition profile of EGFR-TriNKET was characterized by assessing EGFR phosphorylation in vitro and tumor cell growth in vitro and in vivo after exposure to EGFR-TriNKET. Following serum starvation, a panel of wild-type EGFR-expressing cell lines NCI-H292-NucLight Green and were stimulated with EGF, with and without pretreatment using EGFR-TriNKET or cetuximab. Cells were incubated for 1 hour with dose-titrations of EGFR-TriNKET or cetuximab before 20-minute stimulation with EGF. After incubation, cells were prepared for detection of phosphorylated EGFR (pEGFR[Tyr1068]) by AlphaLISA®. As shown in FIG. 23A (NCI-H292-NucLight Green (lung carcinoma) cells) and FIG. 23B (FaDu (head and neck squamous cell carcinoma [HNSCC], hypopharyngeal squamous cell carcinoma subset) cells), cetuximab strongly inhibited EGF-induced EGFR phosphorylation in the cell lines tested. In comparison, EGFR-TriNKET demonstrated a slightly reduced ability to inhibit pEGFR.

To examine the effect of EGFR-TRINKET-mediated signal inhibition of EGFR phosphorylation on tumor cell proliferation, the growth of wild-type EGFR-expressing tumor cells NCI-H292-NucLight Green or FaDu was measured over 72 hours in the presence of dose-titrations of EGFR-TriNKET, cetuximab, or panitumumab starting at 1000 or 200 nM in 1:5 dilutions in complete culture media without addition of exogenous EGFR ligands. Cell growth was monitored over time using an IncuCyte Live-Cell Imager to obtain phase contrast images of each well. The extent of cell growth inhibition was quantified by dividing the difference between the average value of untreated controls and the confluence of each treated sample well value by the average value of untreated controls after normalization to the initial time point (FIG. 24A shows inhibition of proliferation of NCI-H292-NucLight Green cells; FIG. 24B shows inhibition of proliferation of FaDu cells). Both cetuximab and panitumumab showed potent growth inhibition of tumor cells. EGFR-TriNKET showed weaker, but still considerable, growth inhibition, with an EC₅₀ ranging from 1.7 to 3.9 nM. As a monovalent binder, the reduced signal inhibition by EGFR-TriNKET may be attributed to: (1) a decreased ability to effectively compete with EGFR ligands, or (2) a reduced capacity to induce internalization and degradation of EGFR compared to that of bivalent binders like cetuximab and panitumumab.

In summary, EGFR-TriNKET was less active than other antagonistic antibodies in vitro, including cetuximab, in assays where either exogenous EGFR ligand induces EGFR signaling or endogenous EGFR signaling drives cell proliferation.

### EGFR-TriNKET Stimulates NK Cell-Mediated Cytotoxicity of EGFR-Expressing Tumor Cells

The ability of EGFR-TriNKET to enhance NK cell activity was investigated using overnight-rested primary human NK cells from 5 healthy human donors on a panel of EGFR-expressing tumor cell lines. Donors expressing the high-affinity CD16a variant F158V (heterozygous [V/F] or homozygous [V/V]), as well as those with only the low-affinity CD16a variant (F/F), were represented among the donors used to characterize the activity of EGFR-TriNKET. NK cell-mediated cytotoxicity was measured in both short-term (dissociation-enhanced lanthanide fluorescence immunoassay [DELFIA®]) and long-term (IncuCyte Live-Cell analysis) assays.

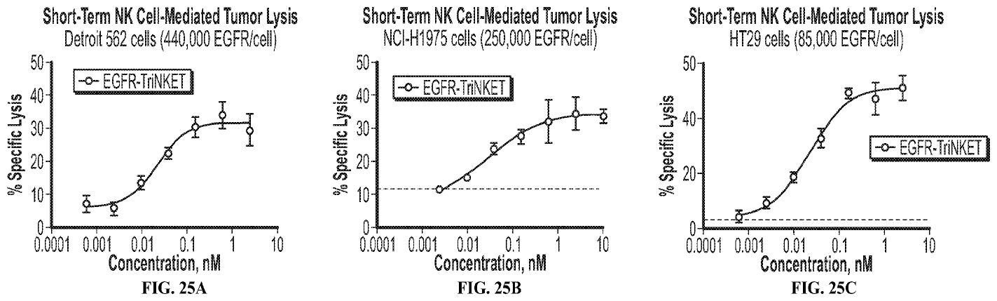

The short-term assay provides a direct assessment of the ability of EGFR-TriNKET to specifically stimulate the cytolytic capacity of NK cells. FIGS. 25A-25C shows the short-term, EGFR-TriNKET-mediated NK cell activity on representative tumor cells lines expressing high (440,000 EGFR/cell), medium (250,000 EGFR/cell), and low (85,000 EGFR/cell) levels of EGFR (FIG. 25A shows lysis of Detroit 562 (pharyngeal carcinoma) cells; FIG. 25B shows lysis of NCI-H1975 (non-small-cell lung cancer [NSCLC] adenocarcinoma) cells; FIG. 25C shows lysis of HT29 cells). Dose-response curves were fit with a nonlinear 4-parameter regression model in GraphPad Prism. EGFR-TriNKET substantially enhanced rested NK cell-mediated lysis of all EGFR-expressing tumor cell lines tested, representing multiple indications of unmet need (Table 17). For each NK cell donor and EGFR-expressing tumor cell line pairing, EGFR-TriNKET enhanced lysis, with a sub-nanomolar EC₅₀. EGFR-TriNKET also enhanced the maximal lysis from 10% to 51% above the basal NK lysis activity in assay wells of effector cells and target cells (E:T) alone without test article. EGFR-TriNKET showed a significant trend of decreasing EC₅₀ values against tumor cell lines with higher cell surface EGFR expression levels (p=0.0034).

TABLE 17
Summary of Short-Term NK Cell-Mediated Cytotoxicity
of EGFR-TriNKET on EGFR⁺ Tumor Cell Lines

| | | | | EGFR-TriNKET | |
|---|---|---|---|---|---|
| | | | Average | | Enhanced |
| | | | EGFR | EC₅₀ | Max |
| Cell Line (n) | | Origin; Details | Per Cell | (nM) | Lysis (%) |
| KYSE-30 | (3) | EsoSCC; EGFRwt | >1,000,000 | 0.0053 ± 0.0040 | 10 ± 2 |
| FaDu | (3) | HNSCC; EGFRwt | 590,000 | 0.011 ± 0.011 | 39 ± 12 |
| Detroit 562 | (3) | HNSCC; EGFRwt | 440,000 | 0.058 ± 0.039 | 22 ± 3 |
| NCI-H292 | (5) | NSCLC; EGFRwt | 400,000 | 0.0088 ± 0.0062 | 51 ± 22 |
| NCI-H1703 | (4) | NSCLC-squam; EGFRwt | 350,000 | 0.022 ± 0.019 | 36 ± 7 |
| 786-O | (4) | RCC; EGFRwt | 320,000 | 0.048 ± 0.038 | 48 ± 25 |
| NCI-H1975 | (4) | NSCLC-adeno; EGFRmut T790M, L858R | 250,000 | 0.10 ± 0.13 | 17 ± 7 |
| HCC4006 | (3) | NSCLC-adeno; EGFRmut L747-E749del, A750P | 140,000 | 0.092 ± 0.041 | 15 ± 8 |
| HCT116 | (4) | CRC; EGFRwt, KRASmut G13D | 88,000 | 0.054 ± 0.052 | 29 ± 4 |
| HT29 | (3) | CRC; EGFRwt, KRASwt | 85,000 | 0.019 ± 0.011 | 24 ± 20 |
| NCI-H2172 | (3) | NSCLC; EGFRwt | 48,000 | 0.079 ± 0.071 | 13 ± 6 |
| BT474 | (3) | BRCA; EGFRwt | 20,000 | 0.29 ± 0.40 | 15 ± 9 |

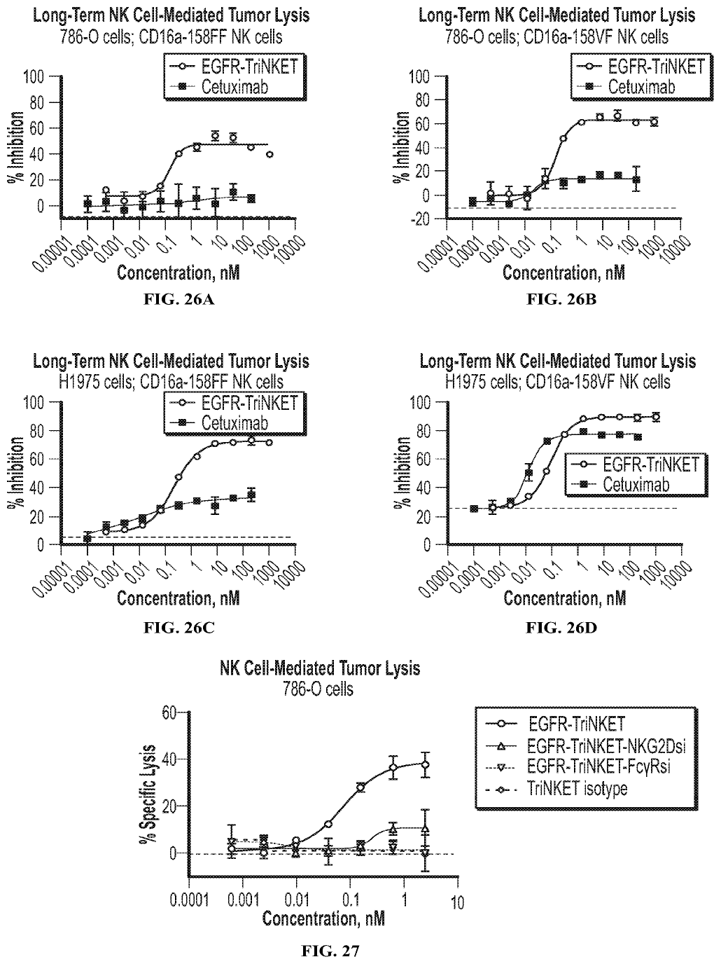

In addition to short-term cytotoxicity assays, long-term activity assays were performed that are more inclusive of multiple mechanisms of activity in the context of tumor cell growth. To increase the translatability of the assay, 50% pooled human serum was included to mimic more physiological conditions, which include the presence of competing IgGs as well as potential EGFR ligands. The robust NK cell-mediated lysis of tumor cells triggered by EGFR-TriNKET in short-term assays translated into substantial inhibition of EGFR-expressing tumor cell outgrowth detected by IncuCyte over a 3-day culture period (FIGS. 26A-26D (FIG. 26A shows lysis of 786-O (renal carcinoma) cells in co-culture with NK cells with only a low-affinity CD16a variant (158FF or F/F); FIG. 26B shows lysis of 786-O cells in co-culture with NK cells with some presence of high-affinity CD16a polymorphism F158V (158VF or V/F); FIG. 26C shows lysis of NCI-H1975 cells in co-culture with NK cells with only a low-affinity CD16a variant (158FF or F/F); FIG. 26D shows lysis of NCI-H1975 cells in co-culture with NK cells with some presence of high-affinity CD16a polymorphism F158V (158VF or V/F)). Primary human NK cells were added for a 5:1 effector-to-target (E:T) ratio to wells that had been pre-seeded for 4 hours with 3000 tumor cells/well transfected to stably express NucLight Green. EGFR-TriNKET and cetuximab were dose titrated from 1000 and 200 nM, respectively, in 1:5 dilutions. Green fluorescent images were taken to assess the growth and survival of tumor cells over time using an IncuCyte® Live-Cell Imager. % Inhibition at 72 hours was calculated by comparing treatment wells to no treatment wells, after normalization to the initial scan to control for variability in cell seeding in the well imaging area. Dose-response curves were fit with a nonlinear 4-parameter regression model in GraphPad Prism. When tested against tumor cell lines representing indications of clinical interest and, in some cases, bearing mutations (e.g., to EGFR and KRAS [Kirsten rat sarcoma viral oncogene homolog]) that have been implicated in resistance to existing EGFR-targeted therapies, paired with healthy human NK cell donors representing multiple CD16a genotypes (F/F, V/F, and V/V), EGFR-TriNKET enhanced maximal inhibition from 32% to 65% above basal inhibition mediated by NK cells against tumor cells alone, with consistently sub-nanomolar potency (Table 18). Maximal inhibition achieved by EGFR-TriNKET exceeded that of cetuximab against each cell line, except for KYSE-30, which expresses extremely high levels of surface EGFR (>1,000,000). Even against a diverse panel of cell lines representing a range of EGFR expression levels, EGFR-TriNKET showed higher levels of maximal tumor growth inhibition over cetuximab at saturation for CD16a V/F or V/V donors (maximal enhanced inhibition values ranged from 43% to 65% for EGFR-TriNKET and from 19% to 62% for cetuximab). The advantage of EGFR-TriNKET was especially pronounced for F/F donors, for whom cetuximab activity was in some cases barely above background (when KYSE-30 cells are excluded, the mean maximal enhanced inhibition values were from 32% to 57% for EGFR-TriNKET and from 9.5% to 24% for cetuximab). Treatment with EGFR-TriNKET and cetuximab alone (in the absence of NK cells) did not result in growth inhibition in the cell lines tested, supporting the conclusion that the bulk of the NK cell- and EGFR-TriNKET-dependent activity observed can be attributed to NK cell lysis potently enhanced by EGFR-TriNKET over time.

TABLE 18
Summary of Long-Term NK Cell-Mediated Cytotoxicity of EGFR-TriNKET on EGFR⁺ Tumor Cell Lines

| | | | | EGFR-TriNKET | | Cetuximab | |
|---|---|---|---|---|---|---|---|
| | | | NK cell | | Max Enhanced | | Max Enhanced |
| NucLight Green | | Average EGFR | CD16a AA158 | EC₅₀ | Inhibition | EC₅₀ | Inhibition |
| Cell Line | Origin; Details | Per Cell | Genotype (n) | (nM) | (%) | (nM) | (%) |
| KYSE-30 | EsoSCC; EGFRwt | >1,000,000 | F/F (n = 2) | 0.015 ± 0.004 | 58 ± 17 | 0.032 ± 0.008 | 54 ± 15 |
| | | | V/F (n = 1) | 0.013 | 65 | 0.029 | 62 |
| 786-O | RCC; EGFRwt | 660,000 | F/F (n = 2) | 0.24 ± 0.14 | 32 ± 11 | 10 ± 12 | 11 ± 6 |
| | | | V/F (n = 1) | 0.15 | 64 | 0.023 | 19 |
| NCI-H1975 | NSCLC-adeno; | 210,000 | F/F (n = 2) | 0.23 ± 0.01 | 57 ± 8 | 0.018 ± 0.013 | 26 ± 3 |
| | EGFRmut T790M, L858R | | V/F (n = 1) | 0.090 | 63 | 0.013 | 51.9 |
| HCT116 | CRC; EGFRwt, | 67,000 | F/F (n = 2) | 0.28 ± 0.10 | 37 ± 5 | 0.032 ± 0.042 | 9.5 ± 6.8 |
| | KRASmut G13D | | V/V (n = 1) | 0.21 | 43 | 0.0060 | 20 |

As the proposed benefit of EGFR-TriNKET over cetuximab stems from its ability to engage NKG2D on NK cells in addition to CD16a, the advantage of EGFR-TriNKET over cetuximab in enhancing NK cell-mediated control of EGFR-expressing tumor cells is especially pronounced in clinically relevant contexts where the relative contribution of CD16a engagement alone is lower, such as with low-affinity CD16a (F/F) and/or in the presence human serum, which contains high concentrations of IgGs that compete for CD16a binding.

Taken together, short- and long-term assays demonstrate the strong activity and versatility of EGFR-TriNKET in enhancing the ability of primary NK cells donors to lyse diverse EGFR-expressing tumor cells.

### EGFR-TriNKET Function is Dependent on Co-Engagement of EGFR, NKG2D, and CD16a

To assess the contribution of each of the TriNKET's targeting arms to the overall activity of the molecule, loss-of-function mutants of EGFR-TriNKET were generated in which each of the targeting arms was disabled. EGFR-TriNKET-FcγRsi encodes mutations in the CH2 domain of the Fc constant region that prevent FcγR binding, including silencing CD16a interactions. With EGFR-TriNKET-NKG2Dsi, the light chain for the NKG2D-engaging arm has been swapped for a non-binding light chain, which abrogates NKG2D agonism. The TriNKET isotype control possesses fully functional NKG2D- and FcγR-binding domains, but the EGFR-targeting arm has been replaced with a binding domain that targets respiratory syncytial virus (RSV).

Loss-of-function mutants were used to assess the contribution of each of functional arms to the potency of EGFR-TriNKET (FIG. 27). Test articles were dose titrated from 2.5 nM in 1:4 dilutions. Each point and error bars represent mean and standard deviation (SD), respectively, of specific lysis from triplicate co-culture wells with a different test article. Dose-response curves were fit with a nonlinear 4-parameter regression model in GraphPad Prism Compared to EGFR-TriNKET, all loss-of-function mutants of EGFR-TriNKET showed reduced NK cell-mediated lysis of 786-O renal carcinoma cells, demonstrating that each individual targeting arm is required for the full potency of EGFR-TriNKET. In these experiments, there was no killing above E:T background in the absence of EGFR-targeting with the TriNKET isotype control. Activity was also lost when FcγR binding was prevented (EGFR-TriNKET-FcγRsi), while the NKG2D-silent molecule (EGFR-TriNKET-NKG2Dsi) exhibited a reduction in potency and maximal lysis compared to that of EGFR-TriNKET. Taken together, this experiment demonstrates that EGFR-TriNKET stimulated rested human NK cells via CD16a engagement alone, but co-engagement of CD16a and NKG2D resulted in a substantial enhancement in activity.

### EGFR-TriNKET Stimulates NK Cell IFNγ Production and Degranulation

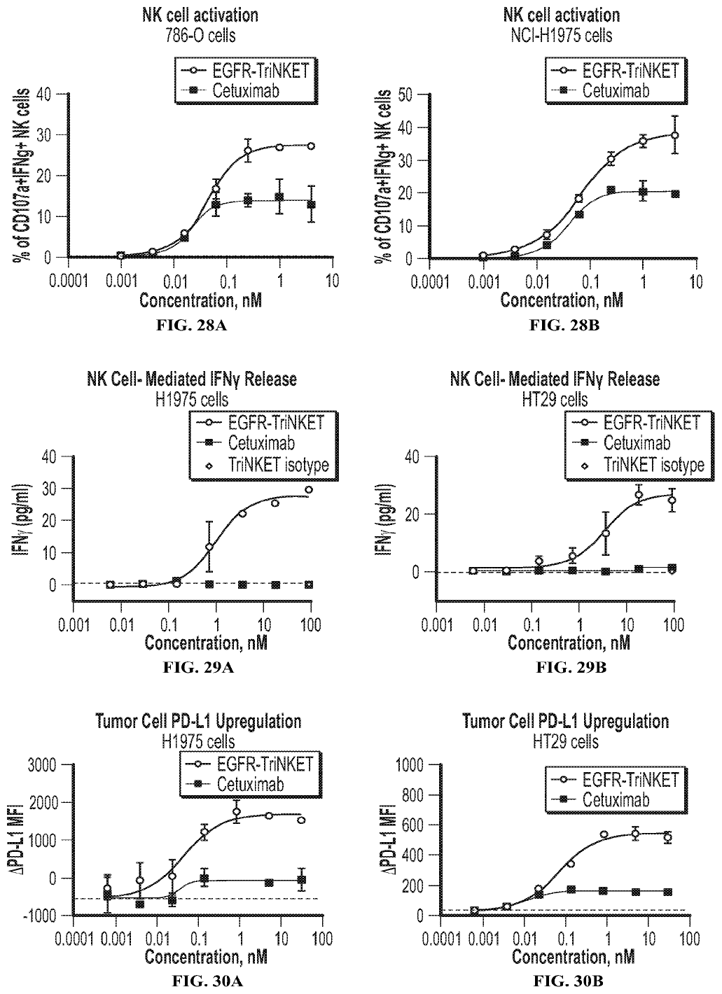

In addition to direct lysis of target cells, NK cells produce cytokines upon activation. Therefore, interferon gamma (IFNγ) production and degranulation (as measured by expression of CD107a) by NK cells were evaluated after PBMCs were co-cultured with EGFR-expressing cancer cells in the presence of EGFR-TriNKET or cetuximab (FIGS. 28A and 28B (FIG. 28A shows degranulation and cytokine production by human NK cells in co-culture with 786-O (renal cell carcinoma) target cells at a 4:1 effector-to-target (E:T) ratio; FIG. 28B shows degranulation and cytokine production by human NK cells in co-culture with NCI-H1975 target cells at a 2:1 E:T)). Overnight-rested PBMCs were placed into co-culture with tumor cells for 4 hours with EGFR-TriNKET or cetuximab. Test articles were dose titrated from 4 nM in 1:4 dilutions. Brefeldin-A, monensin, and interleukin (IL)-2 were included to increase the dynamic range of detected activation. For CD45⁺CD14−CD20−CD3⁻CD56+ NK cells, IFNγ and CD107a gates were drawn based on negative controls and applied to all other samples. Where non-zero, E:T no treatment background activation is marked with a dotted line. Dose-response curves were fit with a nonlinear 4-parameter regression model in GraphPad Prism. Rested NK cells within PBMCs from healthy donors co-cultured with EGFR-expressing tumor cells alone showed little basal induction of CD107a degranulation or intracellular IFNγ accumulation. However, the addition of EGFR-TriNKET resulted in robust induction of degranulation and IFNγ production. EGFR-TriNKET showed higher maximum degranulation and cytokine production compared to that of cetuximab (Table 19).

TABLE 19
Summary of EGFR-TriNKET-Mediated NK Cell Degranulation and Cytokine Production

| | | | EGFR-TriNKET | | Cetuximab | |
|---|---|---|---|---|---|---|
| | | | | Max IFNγ⁺ | | Max IFNγ⁺ |
| | | Average EGFR | EC₅₀ | CD107a⁺ | EC₅₀ | CD107a⁺ |
| Cell Line | Origin; Details | Per Cell | (nM) | NKs (%) | (nM) | NKs (%) |
| 786-O | RCC; EGFRwt | 320,000 | 0.089 ± 0.039 | 21 ± 5 | 0.038 ± 0.024^{a} | 8.7 ± 4.3 |
| NCI-H1975 | NSCLC-adeno; EGFRmut | 250,000 | 0.067 ± 0.014 | 35 ± 2 | 0.049 ± 0.007 | 14 ± 5 |
| | T790M, L858R | | | | | |

The IFNγ concentration in the supernatant of co-cultures of NK cells with EGFR-expressing tumor cells was quantified using AlphaLISA to confirm that the elevated proportion of EGFR-TriNKET-triggered, cytokine-producing NK cells translated into higher IFNγ release. Purified NK cells were pre-stimulated with 10 ng/mL of interleukin (IL)-2 overnight to improve the dynamic range of the assay. In the presence of NCI-H1975 (NSCLC adenocarcinoma) and HT29 tumor cells, EGFR-TriNKET dramatically amplified NK cell IFNγ release above the enhancement observed with cetuximab (FIGS. 29A and 29B (FIG. 29A shows release of IFNγ from human NK cells in the presence of NCI-H1975 cells; FIG. 29B shows release of IFNγ from human NK cells in the presence of HT29 cells); Table 20). Test articles were dose titrated from 30 nM in 1:5 dilutions. Dose-response curves were fit with a nonlinear 4 parameter regression model in GraphPad Prism. As was seen with the cytotoxicity assays, IFNγ release supports the benefit of NKG2D engagement and superior function of EGFR-TriNKET over cetuximab.

TABLE 20
Summary of IFNγ Release From NK Cells in the Presence of EGFR+ Tumor Cells

| | | | EGFR-TriNKET | | Cetuximab | |
|---|---|---|---|---|---|---|
| | | Average EGFR | EC₅₀ | Max IFNγ | EC₅₀ | Max IFNγ |
| Cell Line | Origin; Details | Per Cell | (nM) | ⁽μg/mL⁾ | (nM) | ⁽μg/mL⁾ |
| NCI-H1975 | NSCLC-adeno; EGFRmut | 170,000 | 0.88 ± 0.17 | 29 ± 5 | ND | ND |
| | T790M, L858R | | | | | |
| HT29 | CRC; EGFRwt, KRASwt | 85,000 | 1.6 ± 1.6 | 15 ± 4 | ND | ND |

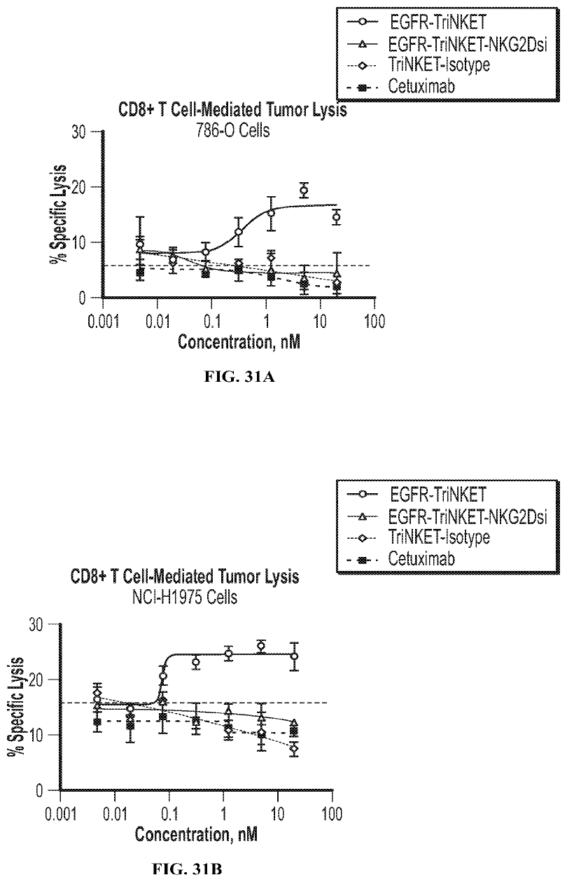

Induction of programmed death-ligand 1 (PD-L1), a ligand of immune checkpoint receptor PD-1, on tumor cells is a well-characterized consequence of IFNγ exposure (Ikeda et al., (2002) Cytokine Growth Factor Rev. 13(2):95-109). To assess whether NK cell stimulation by EGFR-TriNKET could drive elevated PD-L1 expression on EGFR tumor cells, PD-L1 levels were assessed on NCI-H1975 (NSCLC adenocarcinoma) and HT29 tumor cells after 3-day co-culture with overnight-rested primary human NK cells by flow cytometry. Overnight-rested primary human NK cells were added to wells overnight pre-seeded with 5000 target cells for a 5:1 effector-to-target (E:T) ratio. EGFR-TriNKET or cetuximab were dose titrated from 30 nM in 1:6 dilutions. After a 72-hour incubation, differences in PD-L1 median fluorescence intensity (A PD-L1 MFI) were approximated by comparing the PD-L1 MFI values of live target cell from sample wells to target cell-only control wells. Inclusion of EGFR-TriNKET resulted in robust upregulation of surface PD-L1 on both tumor cell lines (FIGS. 30A and 30B (FIG. 30A shows PD-L1 induction on NCI-H1975 cells; FIG. 30B shows PD-L1 induction on HT29 cells)). Dose-response curves were fit with a nonlinear 4-parameter regression model in GraphPad Prism. The maximum increase in PD-L1 signal induced by EGFR-TriNKET was greater than 3-fold that of cetuximab (Table 21), in alignment with superior NK cell activation by EGFR-TriNKET compared to cetuximab.

TABLE 21
Summary of PD-L1 Induction on Tumor Cells in the Presence of NK Cells by EGFR-TriNKET

| | | | EGFR-TriNKET | | Cetuximab | |
|---|---|---|---|---|---|---|
| | | Average EGFR | EC₅₀ | Max Δ PD- | EC₅₀ | Max Δ PD- |
| Cell Line | Origin; Details | Per Cell | (nM) | L1 MFI | (nM) | L1 MFI |
| NCI-H1975 | NSCLC-adeno; EGFRmut | 170,000 | 0.070 ± 0.025 | 2000 ± 200 | 0.018 ± 0.017 | 930 ± 460 |
| | T790M, L858R | | | | | |
| HT29 | CRC; EGFRwt, KRASwt | 85,000 | 0.044 ± 0.027 | 560 ± 80 | 0.0063 ± 0.0033 | 250 ± 160 |

NKG2D is expressed on cytotoxic CD8⁺ T cells in addition to NK cells and can be triggered directly by NKG2D stimulation when activated via a T-cell engager (TCE) and/or cytokines. The ability of EGFR-TriNKET to induce killing of EGFR-expressing tumor cells via cytokine-stimulated CD8⁺ T cells was assessed using in vitro-expanded, IL-15-primed CD8⁺ T cells co-cultured with EGFR-expressing tumor cells. CD8⁺ T cells were isolated from Concanavalin A (Con A) and interleukin (IL)-2-activated PBMCs and then expanded and primed for 9 days with IL-15. Test articles were dose titrated from 20 nM in 1:4 dilutions. Dose-response curves were fit with a nonlinear 4-parameter regression model in GraphPad Prism. EGFR-TriNKET showed dose-dependent increases in CD8⁺ T-cell lysis of EGFR-expressing tumor cells, while cetuximab showed no enhancement of CD8⁺ T-cell cytolysis over basal levels (FIGS. 31A and 31B (FIG. 31A shows lysis of 786-O cells; FIG. 31B shows lysis of NCI-H1975 cells); Table 22). In addition, EGFR-TriNKET-NKG2Dsi was unable to increase CD8⁺ T-cell lysis, confirming that CD8⁺ T cells are triggered in vitro directly by NKG2D stimulation.

TABLE 22
Summary of CD8⁺ T-Cell Lysis of EGFR⁺ Target Cells by EGFR-TriNKET

| | | | EGFR-TriNKET | | Cetuximab | |
|---|---|---|---|---|---|---|
| | | Average EGFR | EC₅₀ | Enhanced | EC₅₀ | Enhanced |
| Cell Line | Origin; Details | Per Cell | (nM) | Lysis (%) | (nM) | Lysis (%) |
| 786-O | RCC; EGFRwt | 320,000 | 0.43 ± 0.29 | 11 ± 4 | ND | ND |
| NCI-H1975 | NSCLC-adeno; EGFRmut | 250,000 | 0.17 ± 0.17 | 16 ± 8 | ND | ND |
| | T790M, L858R | | | | | |

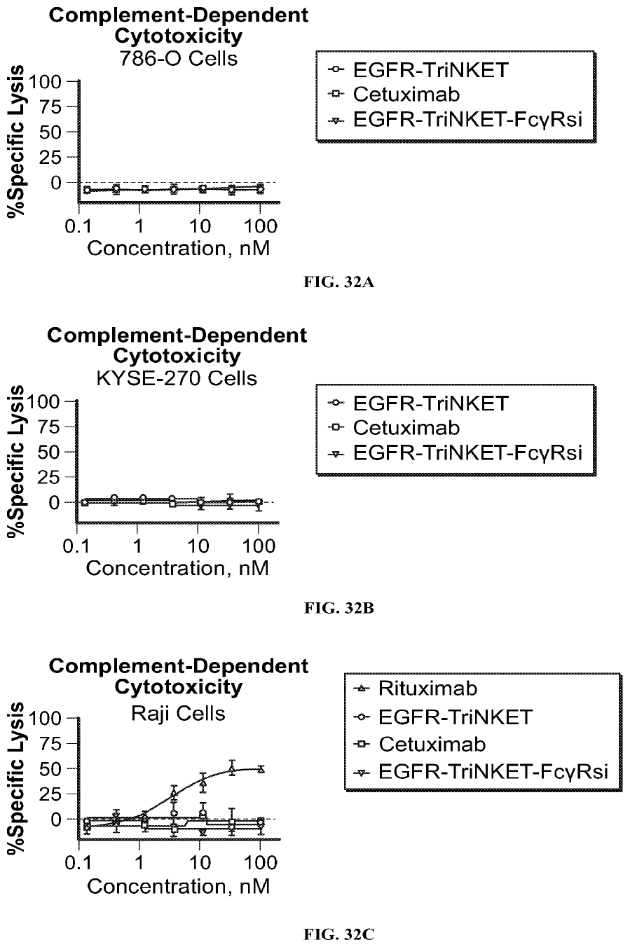

Human IgG1 isotype antibodies have the ability to initiate the complement cascade, leading to complement-dependent cytotoxicity (CDC). Because EGFR-TriNKET contains a human IgG1 Fc domain, the ability of EGFR-TriNKET to stimulate CDC activity was assessed in assays using a panel of human cancer cell lines expressing high levels of cell-surface EGFR. EGFR-expressing tumor cell lines were labeled with DELFIA cytotoxicity reagents and cultured in 5% normal human serum as a source of complement factors. Reconstituted serum with validated hemolytic titer was added to tumor cells to 5% by volume for 45 minutes with dose-titrations of EGFR-TriNKET, cetuximab, or EGFR-TriNKET-FcγRsi. As a positive control for serum activity, Raji cells were treated with a dose-titration of rituximab. Dose-response curves were fit with a nonlinear 4-parameter regression model in GraphPad Prism. EGFR-TriNKET did not stimulate complement-mediated killing of any of the cell lines tested (FIGS. 32A-32C (FIG. 32A shows lysis of 786-O cells; FIG. 32B shows lysis of KYSE-270 (esophageal squamous cell carcinoma) cells; FIG. 32B shows lysis of Raji (Burkitt's lymphoma) cells)). The positive control, rituximab, was incubated with CD20-expressing Raji cells in the presence of human serum and demonstrated CDC activity.

### EGFR-TriNKET has an Increased Ability to Mediate Antibody-Dependent Cellular Phagocytosis

Antibody-dependent cellular phagocytosis (ADCP) is a process by which cells bound by antibody-like molecules are engulfed by phagocytic cells. ADCP can be mediated by monocytes, macrophages, neutrophils, and dendritic cells through recognition of an IgG1 Fc domain by FcγRIIa, FcγRI, and FcγRIIIa, of which FcγRIIa on macrophages represent the predominant pathway. The ability of EGFR-TriNKET to induce ADCP of opsonized target cells was evaluated using MO macrophages obtained from 3 different donors. The MO macrophages were derived from culturing purified CD14⁺ monocytes with macrophage colony-stimulating factor (M-CSF) and subsequently used as effector cells. EGFR-expressing target cells stably expressing NucLight Green were opsonized with test articles and co-cultured with MO macrophages in the presence of 50% pooled human serum. Phagocytosis was analyzed by flow cytometry as CD45⁺ NucLight Green⁺ (double-positive) events. Macrophages differentiated from isolated CD14⁺ monocytes over 7-day culture with macrophage colony-stimulating factor (M-CSF) were added for a 5:1 effector-to-target (E:T) ratio to tumor cells previously transfected to stably express NucLight Green, seeded at 5000 cells/well. EGFR-TriNKET, EGFR-TriNKET-NKG2Dsi, EGFR-TriNKET-FcγRsi, TriNKET isotype, and cetuximab were dose titrated from 100 nM in 1:6 dilutions. Dose-response curves were fit with a nonlinear 4-parameter regression model in GraphPad Prism.

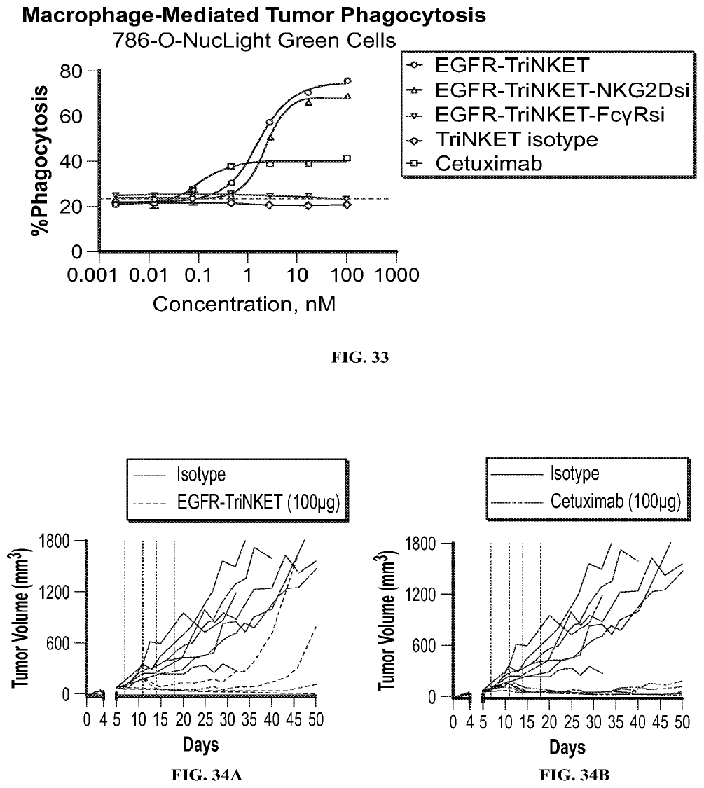

EGFR-TriNKET enhanced phagocytosis of 786-O and NCI-H1975 target cells by MO macrophages to a greater magnitude than that of cetuximab (FIG. 33; Table 23). The enhanced phagocytosis achieved by EGFR-TriNKET may be a result of the elevated cell-binding density of EGFR-TriNKET over cetuximab, which was observed in the same concentration range in binding experiments (FIG. 19A (binding to Detroit 562 tumor cells), FIG. 19B (binding to NCI-H1703 tumor cells), and FIG. 19C (binding to HT29 tumor cells)). In this experiment, EGFR-TriNKET-FcγRsi, which is unable to bind to FcγRs, failed to mediate ADCP of opsonized target cells. The non-EGFR-binding TriNKET isotype also showed negligible activity, while EGFR-TriNKET-NKG2Dsi triggered similar phagocytosis of 786-O tumor cells as compared to that by EGFR-TriNKET. Together, these data suggest that the observed ADCP activity of EGFR-TriNKET is dependent upon its ability to bind EGFR and FcγRs, but not NKG2D.

TABLE 23
Summary of ADCP Activity of EGFR-TriNKET

| | | | EGFR-TriNKET | | Cetuximab | |
|---|---|---|---|---|---|---|
| | | | | Max | | Max |
| NucLight Green | | Average EGFR | EC₅₀ | Phagocytosis | EC₅₀ | Phagocytosis |
| Cell Line | Origin; Details | Per Cell | (nM) | (%) | (nM) | (%) |
| 786-O | RCC; EGFRwt | 660,000 | 2.2 ± 1.1 | 40 ± 11 | 0.11 ± 0.01 | 15 ± 3 |
| NCI-H1975 | NSCLC-adeno; EGFRmut | 210,000 | 2.3 ± 1.1 | 16 ± 12 | ND | 2.5 ± 2.2 |
| | T790M, L858R | | | | | |

### Example 3: In Vivo Pharmacology of EGFR-TriNKET

### EGFR-TriNKET in the NCI-H292 Xenograft Model

In the NCI-H292 xenograft model, EGFR-TriNKET, incorporating: (a) a first polypeptide with the amino acid sequence of SEQ ID NO:167; (b) a second polypeptide with the amino acid sequence of SEQ ID NO:164; and (c) a third polypeptide with the amino acid sequence of SEQ ID NO:165, treatment resulted in a strong anti-tumor response equivalent to that seen with cetuximab. When administered EGFR-TriNKET-FcγRsi or EGFR-TriNKET following depletion of mouse NK cells, anti-tumor efficacy of EGFR-TriNKET in NCI-H292 was maintained, suggesting signal inhibition, rather than ADCC, as an underlying mechanism in this model. No effects on body weights were observed in EGFR-TriNKET-treated animals.

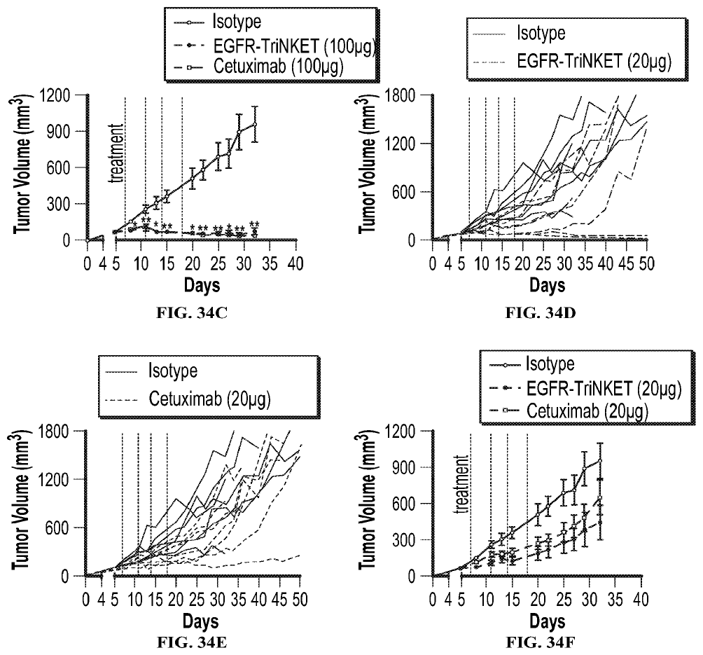

To evaluate the ability of EGFR-TriNKET to elicit anti-tumor activity in vivo, nude mice were injected subcutaneously (SC) with NCI-H292 tumor cells. When tumor volume averaged ˜90 mm³, mice were dosed intraperitoneally (IP) twice weekly for 2 weeks with EGFR-TriNKET or cetuximab at a dose equimolar to 20−(1 mg/kg) or 100-μg (5 mg/kg) EGFR-TriNKET. Nude mice (n=7/group) were engrafted with 4×10⁶ NCI-H292 (lung carcinoma) cells and dosed intraperitoneally (IP) with hIgG1 isotype, EGFR-TriNKET, or cetuximab equimolar to 20 (1 mg/kg) or 100 μg (5 mg/kg) of EGFR-TriNKET. Significance for EGFR-TriNKET and cetuximab relative to isotype control is noted as * (p<0.05) and ** (p<0.01) by 2-way ANOVA. EGFR-TriNKET dosed at 100 μg significantly reduced tumor burden (p<0.05 relative to isotype control) (FIGS. 34A-34F).

A second study was performed to determine the tolerability of EGFR-TriNKET at different dose concentrations. NCI-H292 tumor-bearing mice were dosed IP with 30-, 100-, and 300-pg EGFR-TriNKET twice weekly for 2 weeks. There were no clinical observations nor effects on body weight.

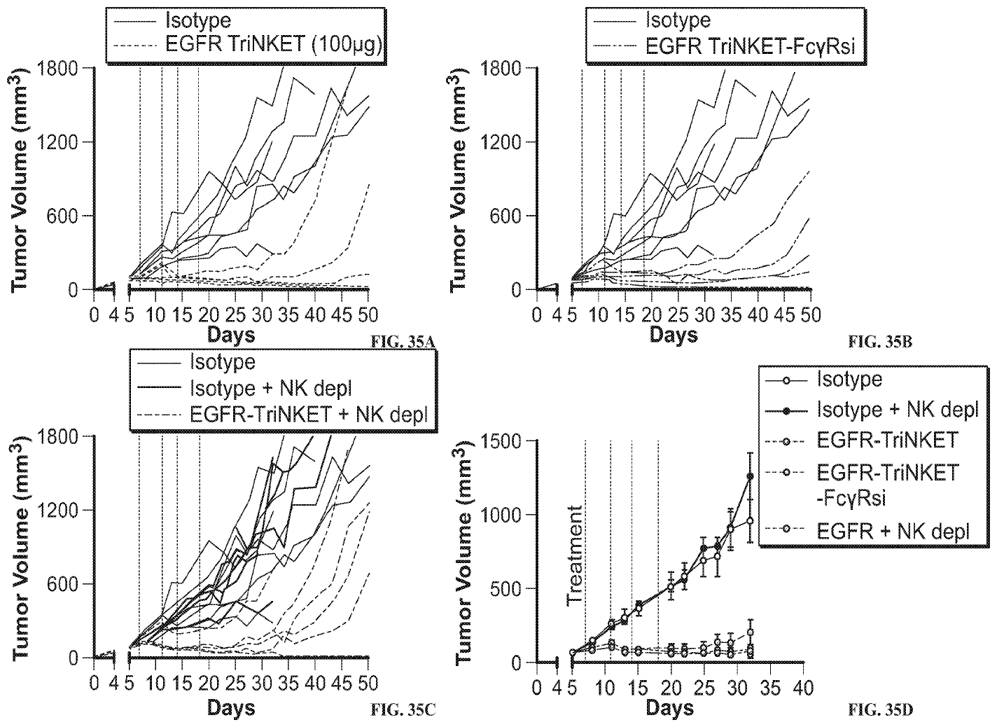

To evaluate the underlying mechanism of the EGFR-TriNKET-mediated anti-tumor response, NCI-H292 tumor-bearing mice were treated with EGFR-TriNKET, EGFR-TriNKET-FcγRsi (ADCC abrogated), or EGFR-TriNKET following depletion of mouse NK cells at a dose equimolar to 100-μg EGFR-TriNKET. Nude mice (n=7/group) were engrafted with 4×10⁶ NCI-H292 (lung carcinoma) cells and dosed intraperitoneally (IP) with hIgG1 isotype, EGFR-TriNKET, EGFR-TriNKET-FcγRsi, or EGFR-TRINKET in combination with NK cell depletion (NK depl). All doses were equimolar to 100 μg (5 mg/kg) of EGFR-TriNKET. To deplete mouse NK cells, mice were administered 25 μL Asialo GM1 antibody on Days 5, 6, 11, 15, 19, 22, 25, and 28 post-tumor inoculation. EGFR-TriNKET treatment resulted in potent anti-tumor responses, the effect of which was not diminished when mice were administered the Fc-silent version of EGFR-TriNKET (FIGS. 35A and 35B). Moreover, NK cell depletion in the presence of EGFR-TriNKET minimally altered anti-tumor efficacy, suggesting signal inhibition, rather than ADCC, as the key driver in mediating anti-tumor responses in the NCI-H292 model (FIGS. 35C and 35D). These results indicate that anti-tumor activity of EGFR-TriNKET in the NCI-H292 xenograft model is driven primarily by EGFR signal inhibition

### EGFR-TriNKET Anti-Tumor Activity in the FaDu Xenograft Model

In the FaDu xenograft model, EGFR-TriNKET treatment resulted in a significant anti-tumor response and was more potent than cetuximab in delaying tumor progression (p<0.05). No effects on body weights were observed in EGFR-TriNKET-treated animals.

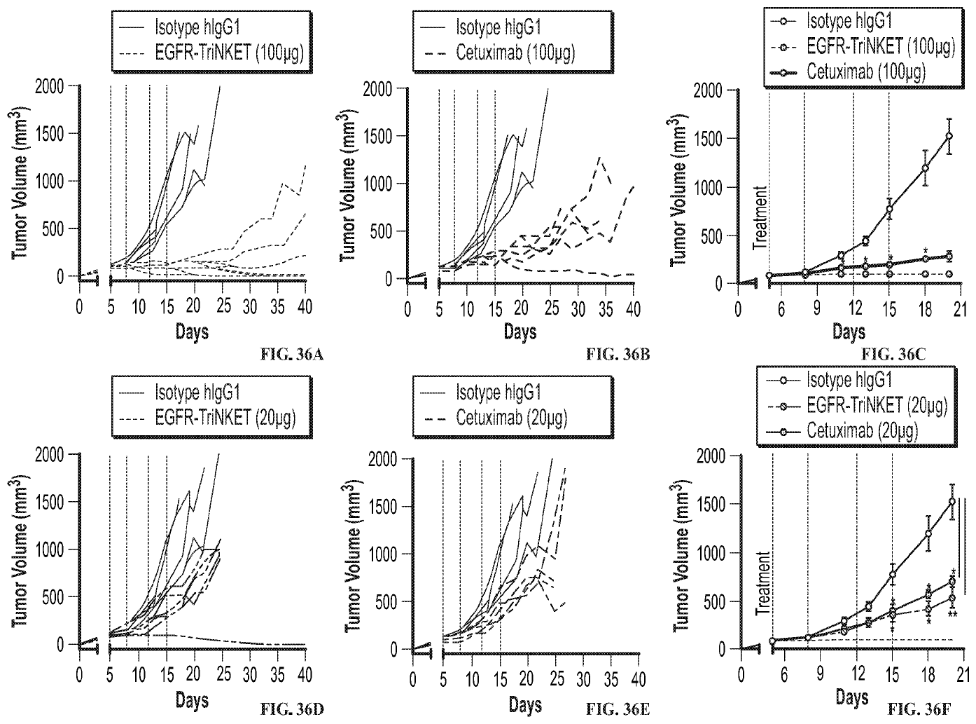

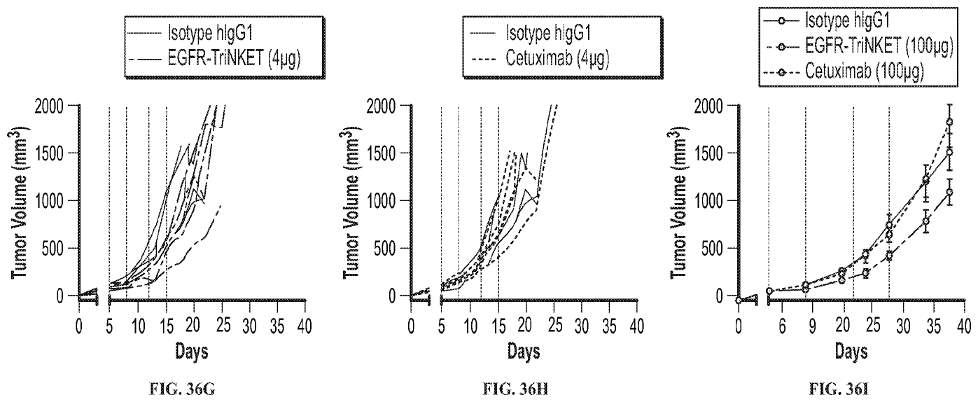

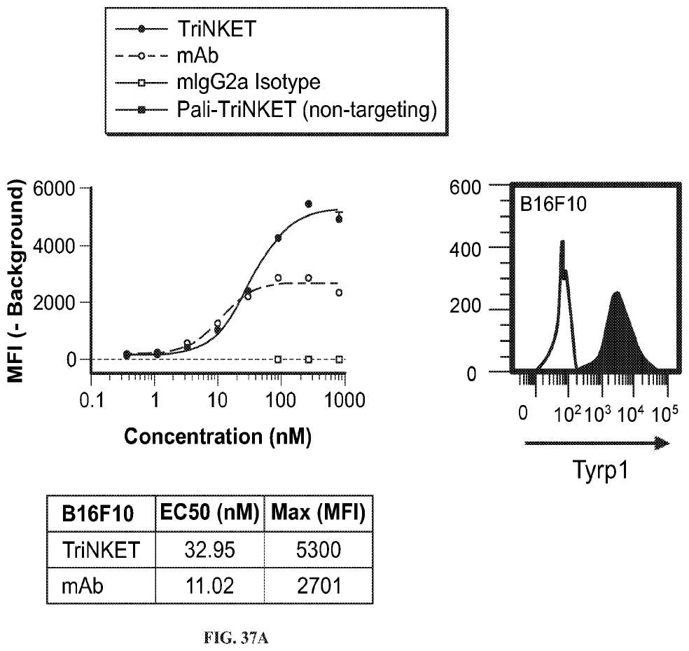

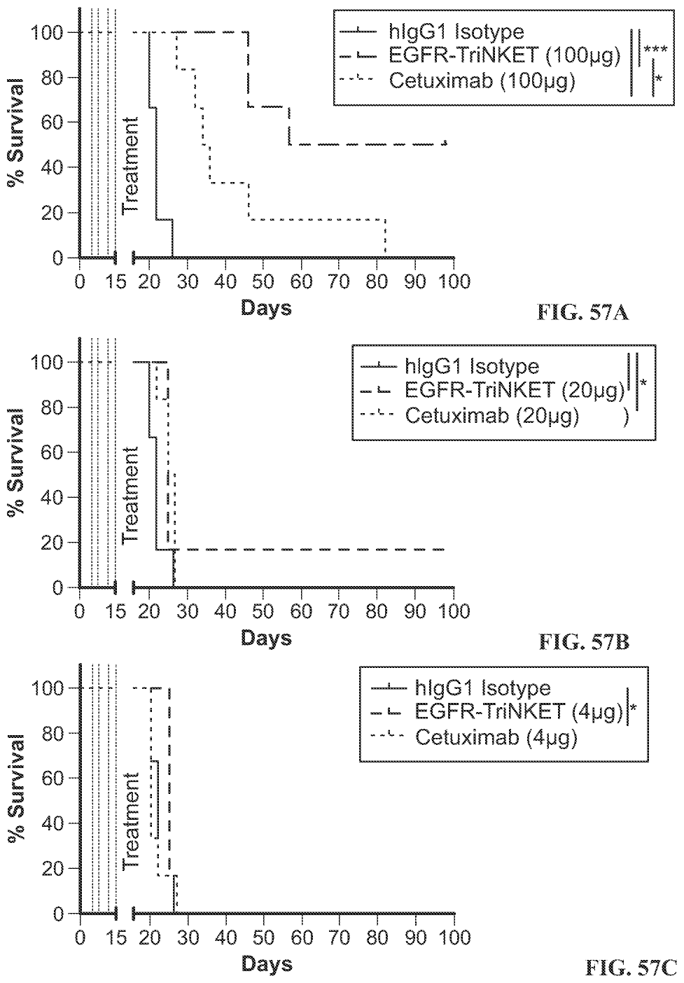

To evaluate the ability of EGFR-TriNKET to elicit anti-tumor activity in vivo in a second xenograft model, nude mice were injected SC with FaDu tumor cells. Nude mice (n=6/group) were engrafted with 2×10⁶ FaDu cells and dosed intraperitoneally (IP) with hIgG1 isotype, EGFR-TriNKET, or cetuximab equimolar to 4 (0.2 mg/kg), 20 (1 mg/kg), or 100 μg (5 mg/kg) of EGFR-TriNKET. When tumor volume averaged ˜95 mm³, mice were dosed IP twice weekly for 2 weeks with EGFR-TriNKET or cetuximab at doses equimolar to 4-, 20-, or 100-μg EGFR-TriNKET. EGFR-TriNKET significantly reduced tumor burden at 20 and 100 μg (p<0.05) and was more potent in mediating anti-tumor responses than cetuximab when dosed at 100 μg (3/6 complete responses [CRs] with EGFR-TriNKET vs 0/6 with cetuximab) (FIGS. 36A-36I and FIGS. 57A-57C). At a 100 μg dose (FIG. 57A), EGFR-TriNKET significantly delayed tumor progression and extended survival compared to that seen in isotype- or Cetuximab-treated animals (p<0.001 and p<0.05, respectively). At a 20 μg dose (FIG. 57B), EGFR-TriNKET significantly delayed tumor progression and extended survival compared to that seen in isotype-or Cetuximab-treated animals (p<0.05 for both comparisons). At a 4 μg dose (FIG. 57C), EGFR-TriNKET significantly delayed tumor progression and extended survival compared to that seen in isotype treated animals (p<0.05). These results indicate that anti-tumor activity of EGFR-TriNKET in the FaDu xenograft model is dose dependent.

### Anti-Tumor Activity of a Surrogate TriNKET in Syngeneic Mouse Models

The lack of a competent immune system needed to support human xenograft models prevents a full assessment of the mechanisms of action of EGFR-TriNKET. Because EGFR-TriNKET is not cross-reactive with mouse NKG2D or EGFR, surrogate TriNKET was developed to further examine in vivo anti-tumor efficacy in syngeneic models (B16F10 and CT26-Tyrp1) in mice that have a fully functional immune system. The surrogate TriNKET utilizes alternative NKG2D and cancer antigen-binding domains because EGFR-TriNKET is not cross-reactive with murine orthologs of EGFR or NKG2D.

With the caveats inherent in making inferences between species, it is reasoned that the use of syngeneic mouse models with fully intact immune systems would recapitulate the immunological mechanisms of action of EGFR-TriNKET with more fidelity than xenograft or humanized mouse models. The murine orthologs of NKG2D and CD16a are considered to have similar expression pattern and function in mice compared to that in humans. However, the NKG2D antibody-binding domain in EGFR-TriNKET does not cross-react with murine NKG2D. To enable in vivo efficacy studies in mice, an alternative antibody to NKG2D was selected, which binds to murine NKG2D. Moreover, a mouse IgG2a isotype was used, which has been found to be most analogous to human IgG1 (Bruhns P et al., (2015) Immunol Rev. 268(1):25).

Given the lack of EGFR cross-reactivity of EGFR-TriNKET, the murine cancer antigen target Tyrp1 was used to minimize immune responses unrelated to the test article. The mAb TA99 binds murine Tyrp1 and has been characterized for its efficacy against B16F10 tumors, which express high levels of Tyrp1 (Hara et al. (1995) Int J Cancer. 61(2):253-60). However, targeting Tyrp1 does not mediate signal inhibition leading to inhibition of tumor cell growth; therefore, the use of Tyrp1 as a surrogate target does not recapitulate the EGFR signal inhibition activity of EGFR-TriNKET.

The surrogate Tyrp1-TriNKET was characterized in vitro using the B16F10 melanoma cell line (endogenously expressing ˜39,000 Tyrp1/cell) and CT26-Tyrp1 colorectal carcinoma cell line (exogenously expressing 6900 Tyrp1/cell via retroviral transduction).

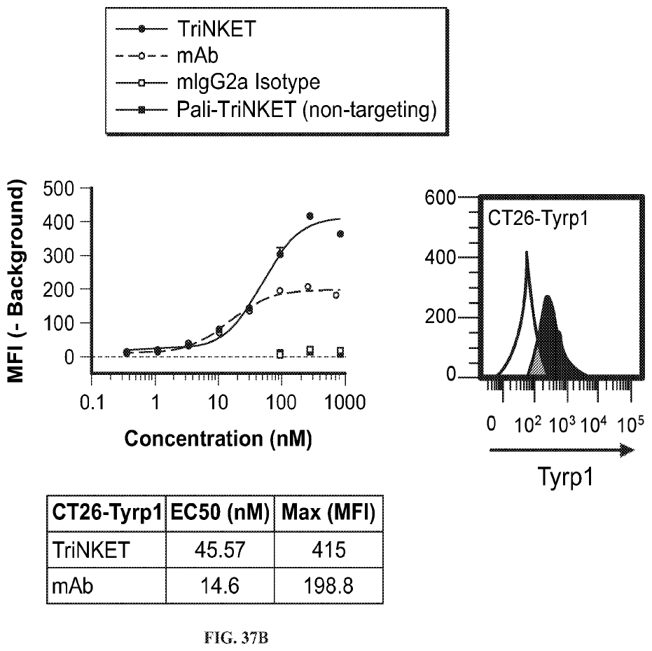

On both mouse tumor cell lines, Tyrp1-TriNKET loaded to a higher maximum than that seen with its parental mAb (TA99) (FIGS. 37A and 37B), comparable to the binding pattern observed with EGFR-TriNKET on human tumor cell lines (FIG. 19A (binding to Detroit 562 tumor cells), FIG. 19B (binding to NCI-H1703 tumor cells), and FIG. 19C (binding to HT29 tumor cells)).

Tyrp1-TriNKET and TA99 parent monoclonal antibody (mAb) were titrated from 800 to 0.37 nM and incubated with B16F10 (melanoma) and CT26-Tyrp1 cells. TriNKET and mAb binding were detected using a fluorophore-conjugated, anti-mouse, IgG-Fc secondary antibody. Cells were analyzed by flow cytometry; mean fluorescent intensity (MFI) was calculated by subtracting the background signal of a nontargeting isotype control (Pali-TriNKET). Cell-binding values and concentration resulting in half-maximal inhibition (EC) were calculated from a 4-parameter, nonlinear regression curve fit.

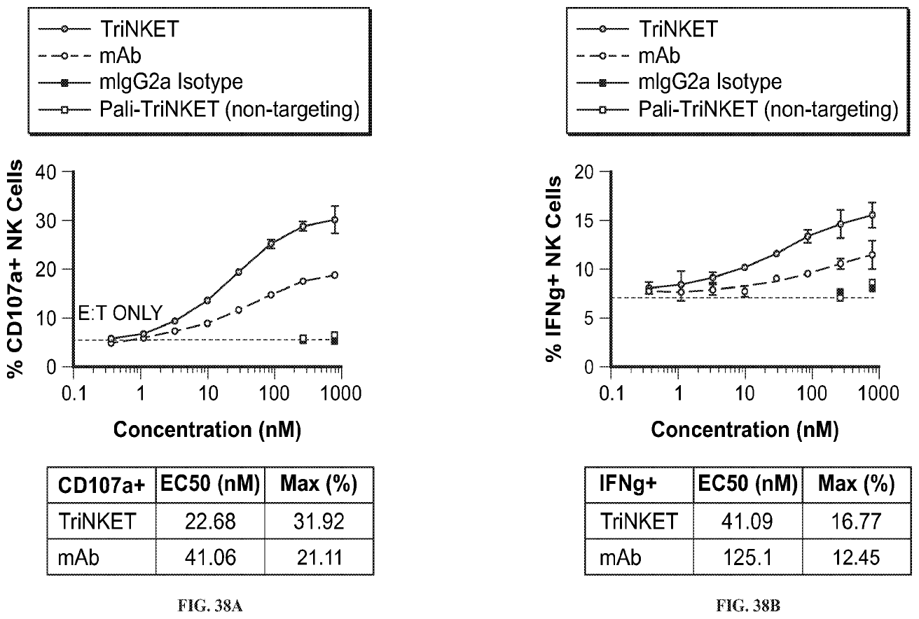

Additionally, the activity of Tyrpl-TriNKET was examined in co-cultures of B16F10 cells and purified mouse NK cells (FIG. 38A shows % CD107a positivity of cells following incubation with indicated proteins; FIG. 38B shows % IFNγ positivity of cells following incubation with indicated proteins). Tyrp1-TriNKET induced NK cell activation, measured as CD107a (degranulation) and IFNγ (cytokine production), to a higher extent than that seen with the TA99 mAb.

Mouse NK cells were purified from spleen and cultured with B16F10 melanoma cells at a 1:1 effector-to-target (E:T) ratio. Eight concentrations of TriNKET or TA99 (parental monoclonal antibody [mAb]) were tested as indicated above. mIgG2a isotype and the nontargeting isotype control (Pali-TriNKET) were used as negative controls at the top 2 concentrations. After 4 hours of culture, cells were prepared for fluorescence-activated cell sorting (FACS) analysis for intracellular IFNγ accumulation and CD107a degranulation. IFNγ⁺ or CD107a⁺ cells were assessed in the NK cell gate (NK1.1⁺CD3⁻). These results indicate that the surrogate mouse TriNKET targeting Tyrpl has a similar in vitro profile to human TriNKETs, including EGFR-TriNKET.

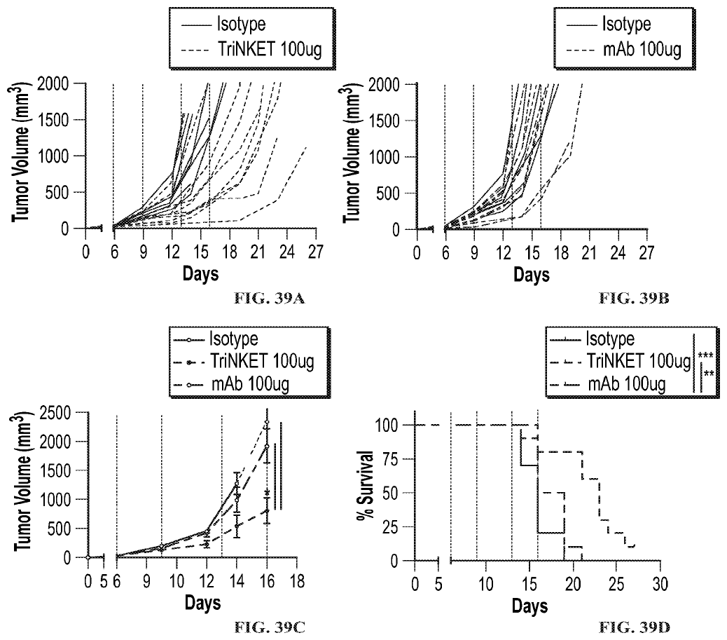

To evaluate the anti-tumor activity of the surrogate Tyrp1-TriNKET in immunocompetent mice, B16F10 tumor-bearing mice were dosed IP twice weekly with isotype, Tyrp1-TriNKET, or its parental mAb (TA99) when tumor volume averaged ˜40 mm³. C57BL/6 mice (n=10/group) were engrafted with 2×10⁵ B16F10 melanoma cells and dosed intraperitoneally (IP) with mIgG2a isotype, Tyrp1-TriNKET, or TA99 (parental monoclonal antibody [mAb]) at 100 μg/dose. Three mice reaching endpoint were removed from the study on Day 14, and mean tumor volume was calculated with the remaining 7 animals. Tyrp1-TriNKET significantly delayed tumor progression and extended survival compared to that seen in isotype-or mAb-treated animals (p<0.01 and p<0.001, respectively) (FIGS. 39A-39D).

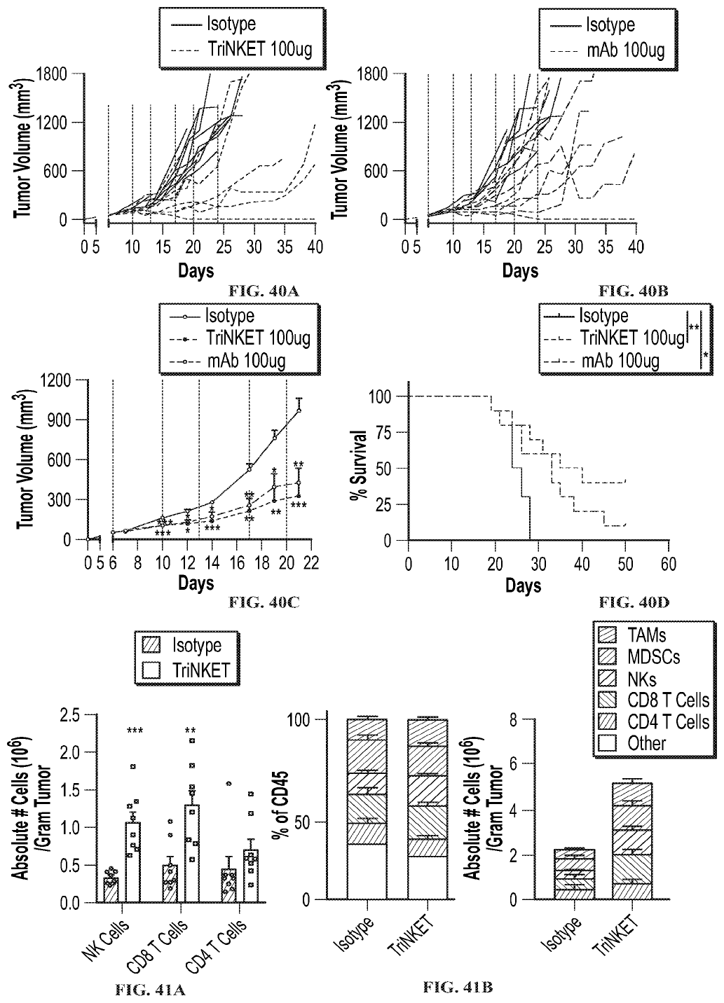

To evaluate the anti-tumor activity of the surrogate Tyrp-1 TriNKET in a second model, CT26-Tyrp1 tumor-bearing mice were dosed IP twice weekly with isotype, Tyrp1-TriNKET, or its parental mAb (TA99) when tumor volume averaged ˜50 mm³. Balb/c mice (n=10/group) were engrafted with 5×10⁵ CT26-Tyrp1 colorectal carcinoma cells and dosed intraperitoneally (IP) with mIgG2a isotype, Tyrpl-TriNKET, or TA99 (parental monoclonal antibody [mAb]) at 100 μg/dose. Two mice with ulcerating tumors were removed from the study on Day 19, and tumor volumes were carried over to Day 21. Tyrp1-TriNKET significantly reduced the outgrowth of CT26-Tyrp1 tumors when compared to isotype-treated animals (p<0.01) and increased survival (p<0.01), with 30% of mice achieving CRs. Unlike in the B16F10 model, TA99 mAb also demonstrated a therapeutic benefit in the CT26-Tyrp1 model, yielding 10% CRs (FIGS. 40A-40D).

### Tyrp-1 Surrogate TriNKET is a Potent Activator of Innate and Adaptive Immunity in the B16F10 Model

To understand the mechanisms of action of TriNKETs in vivo, B16F10 tumor-bearing mice were treated with isotype or Tyrp1-TriNKET either once or every other day for 1 week when tumor volume averaged ˜35 mm³. All treatments were administered at 150 μg. Tumors were analyzed for soluble and cellular changes.

Overall, 5 key observations supporting Tyrp1-TriNKET as a potent activator of innate and adaptive immunity are summarized as follows:

1. Elevated cytokine/chemokine levels in the tumor environment.

2. Expanded NK- and T-cell populations in the tumor environment.

3. Increased activation of immune cells.

4. Increased T-cell memory.

5. Upregulation of checkpoint molecules.

Chemokines and cytokines were measured in serum and B16F10 tumor lysates 24 hours after treatment with mIgG2a isotype, Tyrp1-TriNKET, or TA99 mAb, using a multiplex bead-based technology. A single dose of Tyrp1-TriNKET increased intra-tumoral levels of several cytokines and chemokines (TNFα, CCL4, CCL5, CXCL9, CXCL10). Tyrpl mAb treatment resulted in cytokine/chemokine expression at lower levels.

Immune populations were analyzed in the tumor microenvironment by flow cytometry at 7 days post-treatment start. C57BL/6 mice (n=8-9/group) were inoculated subcutaneously (SC) with 2×10⁵ B16F10 melanoma cells and dosed intraperitoneally (IP) with 150 μg isotype or Tyrp1-TriNKET. Tyrp1-TriNKET treatment significantly increased the number of tumor-infiltrating NK cells and CD8⁺ T cells (p<0.001 and p<0.01, respectively) as compared to isotype-treated mice (FIGS. 41A-41B).

Furthermore, Tyrp1-TriNKET treatment significantly increased the activation of effector cells (p<0.01), as indicated by the upregulation of activation markers CD25, CD69, and 4-1BB on NK cells and CD8⁺ and CD4⁺ T cells as compared to the control group at 7 days post-treatment. Administration of Tyrp1-TriNKETs also significantly increased the frequencies of central memory and effector memory T cells in the tumor microenvironment at 7 days post-treatment (p<0.01).

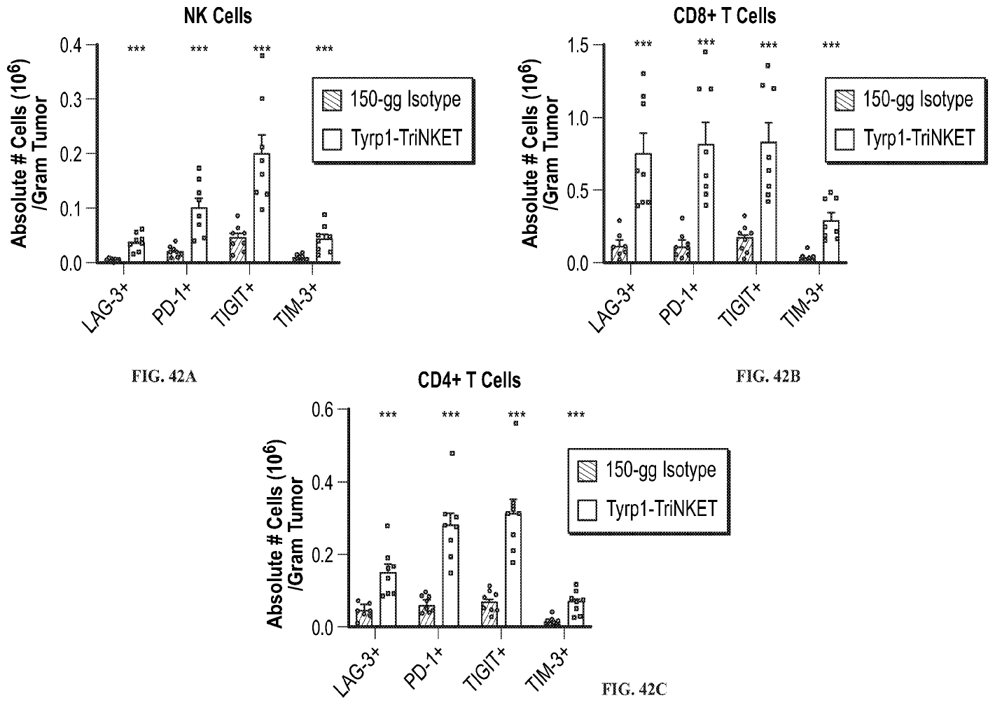

Moreover, significantly elevated levels of checkpoint molecules (PD-1, LAG-3 [lymphocyte activation gene-3], TIGIT [T-cell immunoglobulin and ITIM domain], and TIM-3 [T-cell immunoglobulin and mucin domain-3]) were observed on immune cells at 7 days post-treatment (FIGS. 42A-42C). C57BL/6 mice (n=8-9/group) were inoculated subcutaneously (SC) with 2×10⁵ B16F10 melanoma cells and dosed intraperitoneally (IP) with 150-gg isotype or Tyrpl-TriNKET.

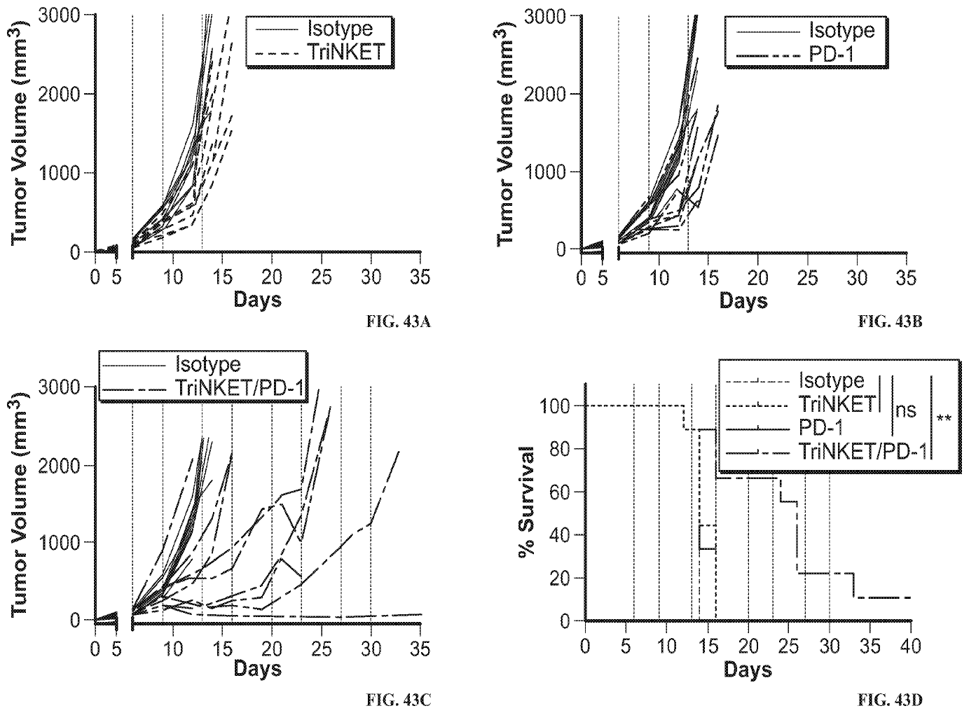

Because the Tyrp1-TriNKET upregulated the expression of checkpoint molecules, combination therapy of Tyrp1-TriNKET and PD-1 blockade was performed in the B16F10 tumor model using established tumors, which are known to be largely resistant to PD-1 blockade (Mosely et al. (2017) Cancer Immunol Res. 5(1):29-41), in order to analyze whether the anti-tumor immune response could be amplified. When tumor volume averaged ˜100 mm³, B16F10 tumor-bearing mice were dosed IP twice weekly with either 100 μg Tyrp1-TriNKET or 200 μg anti-PD-1 as monotherapies or in combination. C57BL/6 mice (n=10/group) were engrafted with 2×10⁵ B16F10 melanoma cells and dosed intraperitoneally (IP) with 100-μg isotype, 100 μg Tyrp1-TriNKET, 200-μg anti-PD-1, or the combination of Tyrp1-TriNKET and anti-PD-1. While the individual therapies were minimally efficacious against large B16F10 tumors, combination of Tyrp1-TriNKET with PD-1 blockade extended the duration of anti-tumor responses (FIGS. 43A-43C). Survival with Tyrp1-TriNKET in combination with PD-1 blockade was significantly (p<0.01) prolonged compared to either monotherapy or in isotype control-treated mice. Notably, despite the synergistic efficacy, the regimen of TriNKET and anti-PD-1 combination therapy was well tolerated by B16F10 tumor-bearing mice. There were no signs of additive or synergistic toxicity in the combination of TriNKET and PD-1 blockade. There were neither clinical observations nor effects on body weight.

In summary, this study provides evidence that TriNKET therapy can sensitize checkpoint-resistant tumors, and combination of TriNKET and PD-1 blockade resulted in robust anti-tumor efficacy. Such combination therapy could be an effective anti-tumor therapy in the clinic.

### Secondary Pharmacology

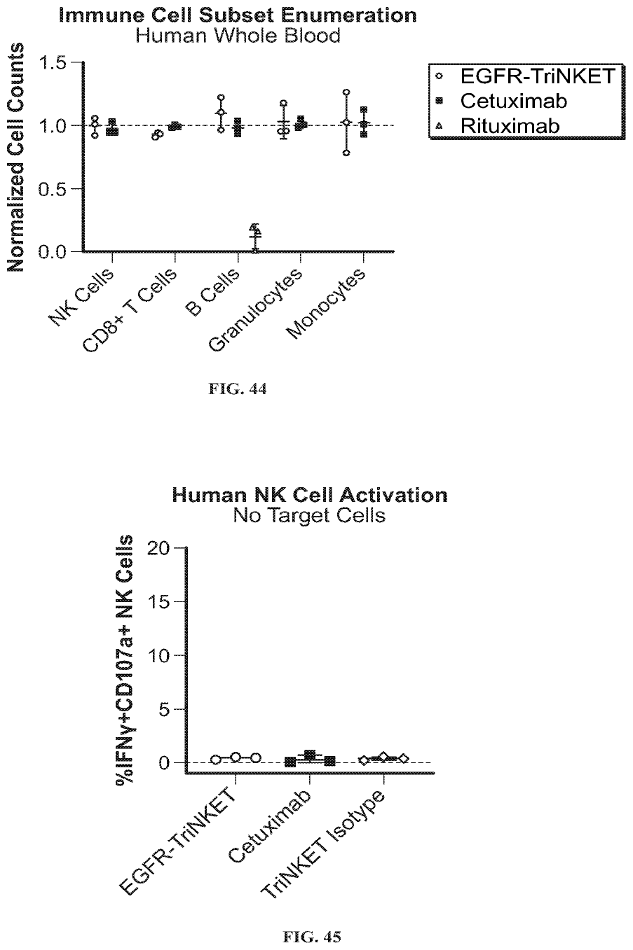

To examine the impact of EGFR-TriNKET on peripheral immune cells, human whole blood was incubated for 24 hours with EGFR-TriNKET or cetuximab, and cell subpopulations (B cells, NK cells, CD8⁺ T cells, monocytes, and granulocytes) were subsequently analyzed by FACS (FIG. 44). Rituximab, which is known to deplete B cells under these conditions, was used as a positive control. Cell counts for each of the populations in treated samples were compared to CD4⁺ T-cell counts to control for bulk well technical variation before normalization to untreated control wells.

Normalized immune cell counts from 3 whole blood samples showed that rituximab resulted in 87.9% depletion of B cells. In contrast, EGFR-TriNKET did not cause any substantial changes in cell frequencies in the samples, similar to the profile of cetuximab. Thus, while EGFR-TriNKET has the ability to engage NKG2D and Fcγ receptors, there is no indication of immune cell bridging or subsequent depletion, suggesting that EGFR-TriNKET cytotoxic activity requires EGFR targeting.

Since EGFR-TriNKET has the potential to bind transiently to various immune cell subsets via FcγR and NKG2D in the periphery, the influence of EGFR-TriNKET on human NK cell activation within PBMCs was examined in the absence of EGFR-expressing target cells. PBMCs were incubated for 4 hours with EGFR-TriNKET, cetuximab, or a TriNKET isotype control, and the NK cells were subsequently analyzed by FACS, using CD107a and IFNγ accumulation as indicators of NK cell activation (FIG. 45). Degranulation and cytokine production by human NK cells were measured among PBMCs in the absence of EGFR-expressing target cells. Overnight-rested PBMCs from 3 healthy human donors were placed into culture for 4 hours with 4 nM of EGFR-TriNKET, cetuximab, or a TriNKET isotype control. Brefeldin-A, monensin, and interleukin (IL)-2 were included to increase the dynamic range of detected activation. For CD45⁺CD14⁻CD20⁻CD3⁻CD56+ NK cells, IFNγ and CD107a gates were drawn based on negative controls and applied to all other samples. Neither EGFR-TRINKET nor cetuximab activated NK cells, and CD107a and IFNγ levels were comparable to those seen in PBMCs alone or when incubated with the non-EGFR-targeting TriNKET isotype control. However, in assays with EGFR-expressing tumor cells present, EGFR-TriNKET triggered robust NK cell activation within PBMC samples from the same healthy human donors, supporting that the activity of EGFR-TriNKET requires the localization and stable association with EGFR-expressing tumor cells.

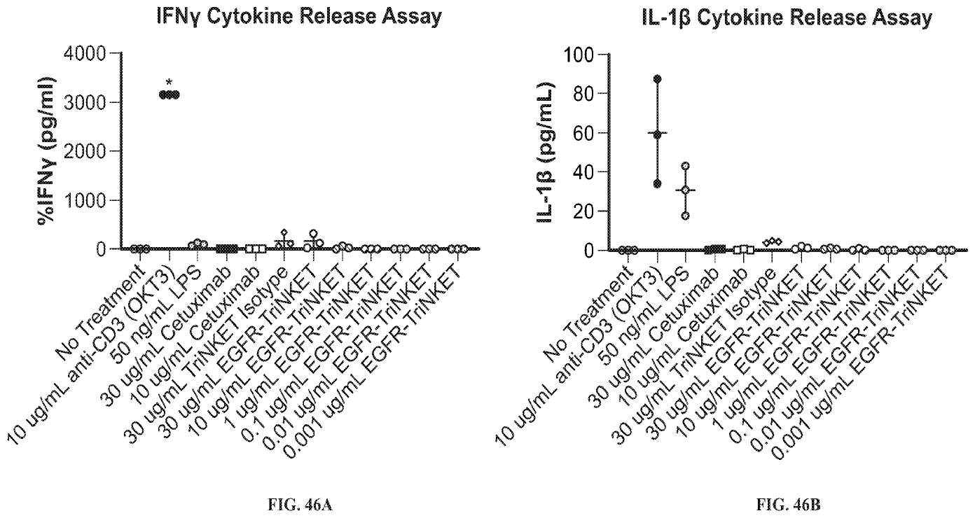

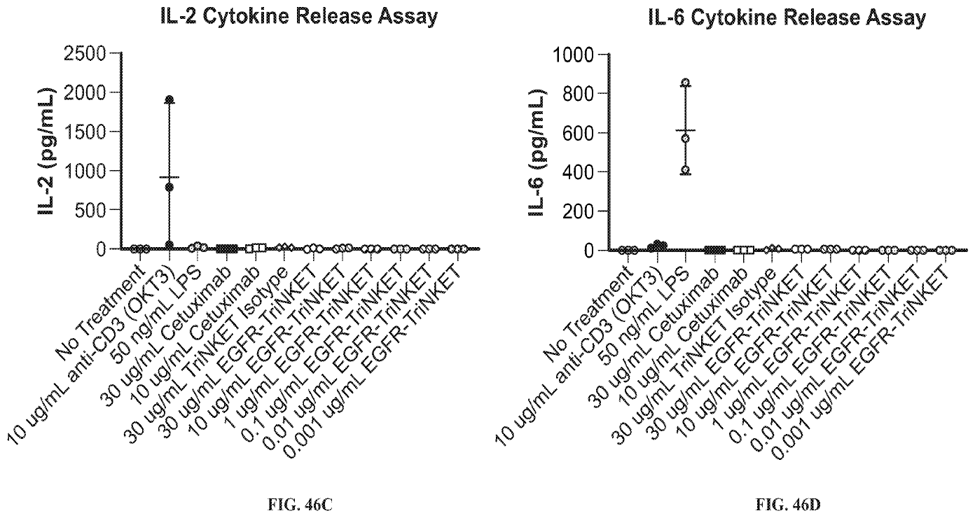

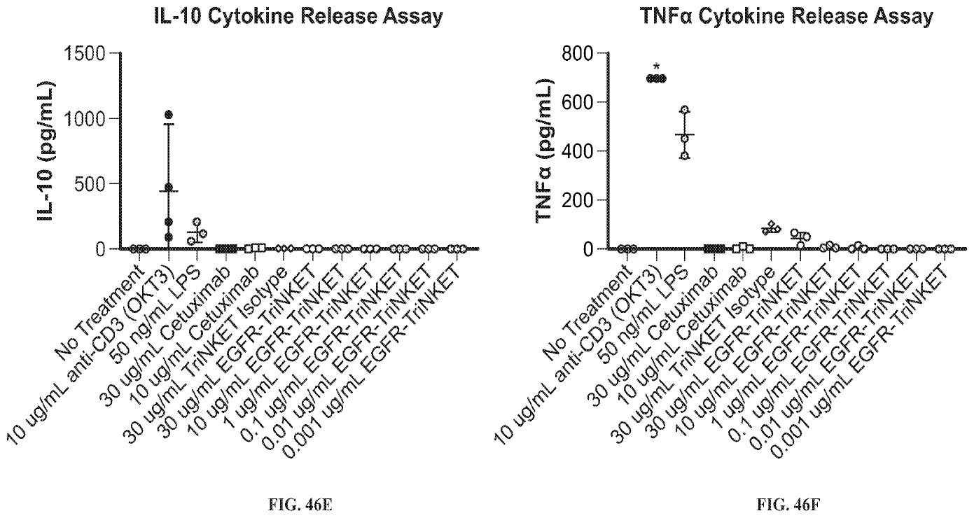

One of the most dangerous side effects caused by the administration of immune-modulating therapies is the potential for cytokine release syndrome (CRS), which is a large, rapid release of cytokines into the blood from immune cells after treatment. The ability of EGFR-TriNKET to induce cytokine release in vitro was assessed using plate-bound, immobilized EGFR-TriNKET with human PBMCs. Cytokine release assays were performed by adding PBMCs to plates wet-coated with research-grade EGFR-TriNKET, cetuximab, or control molecules. After a 48-hour incubation, the concentrations of cytokines (including IFNγ, IL-1β, IL-2, IL-6, IL-10, and TNFα) present in the supernatants were measured using a Meso Scale Discovery (MSD) assay. Between the positive controls, culture with plate-bound anti-CD3 antibody (clone OKT-3) resulted in high levels of release of IFNγ, IL-1β, IL-2, IL-10, and TNFα, while lipopolysaccharide (LPS) triggered substantial IL-1β, IL-6, and TNFα release (FIGS. 46A-46F), consistent with reported values. In contrast, incubation of PBMCs in the presence of EGFR-TriNKET only induced secretion of low levels of IFNγ and TNFα, to a similar extent as that seen with cetuximab. This level of cytokine response was equivalent to that observed with the TriNKET isotype and multiple orders lower than levels triggered by CD3 engagement (here underestimated because of positive control samples exceeding the upper limit of detection [ULOD] of the assay). These data suggest a limited capacity to trigger cytokine release in vitro in the absence of malignant EGFR-expressing cells.

A non-GLP in vitro study evaluating cytokine release in human PBMCs is conducted using GLP-grade EGFR-TriNKET representative of clinical material. The PBMCs are isolated from whole blood from 10 healthy human donors and incubated for 48 hours with EGFR-TriNKET (0.1 to 100 μg/mL) and appropriate negative and positive controls. Stimulation is performed in soluble and immobilized (wet-coated) formats. The cell supernatant is collected and analyzed in an 8-plex Luminex assay for IL-1B, IL-2, IL-6, IL-10, IL-12(p70), TNFα, IFNγ, and granulocyte colony stimulating factor (G-CSF), and in a 1-plex Luminex assay for IL-8.

### Effects of EGFR-TriNKET on Human Primary Keratinocytes In Vitro

Dermatologic toxicities are the most common adverse events associated with the existing anti-EGFR therapies (Huang et al., (2020) Sci Rep. 10(1):4803). Inhibition of EGFR signaling in keratinocytes, the major constituent of skin epithelium, induces apoptosis and modifies cytokine production, resulting in cell death and persistent inflammation in the skin, respectively (Lacouture, (2006) Nat Rev Cancer. 6(10):803-12). These 2 independent events lead to chronic dermatologic toxicities. The potential for these toxicities with EGFR-TriNKET was explored in vitro using human primary keratinocytes.

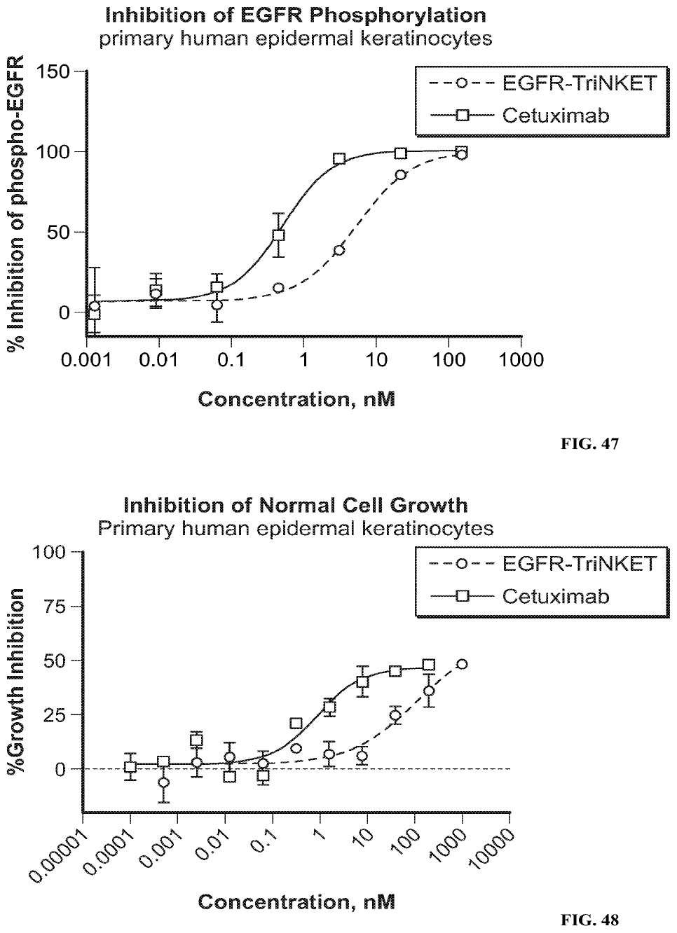

This study evaluated the ability of EGFR-TriNKET to inhibit EGF-induced EGFR signaling in human primary keratinocytes, compared to that of cetuximab. Human primary keratinocytes were starved for 2 hours and then preincubated with EGFR-TRINKET and cetuximab. The cells were then stimulated with EGF for 20 minutes. Cell lysates were prepared for phosphoEGFR detection by AlphaLISA. As shown in FIG. 47, cetuximab strongly inhibited EGF-induced EGFR phosphorylation (EC₅₀=0.5 nM), while EGFR-TriNKET had a reduced impact (EC₅₀=5.3 nM). Similar to data generated using tumor cell lines, these data suggest that EGFR-TriNKET has a reduced ability to inhibit EGFR signaling in human primary keratinocytes compared to that of cetuximab.

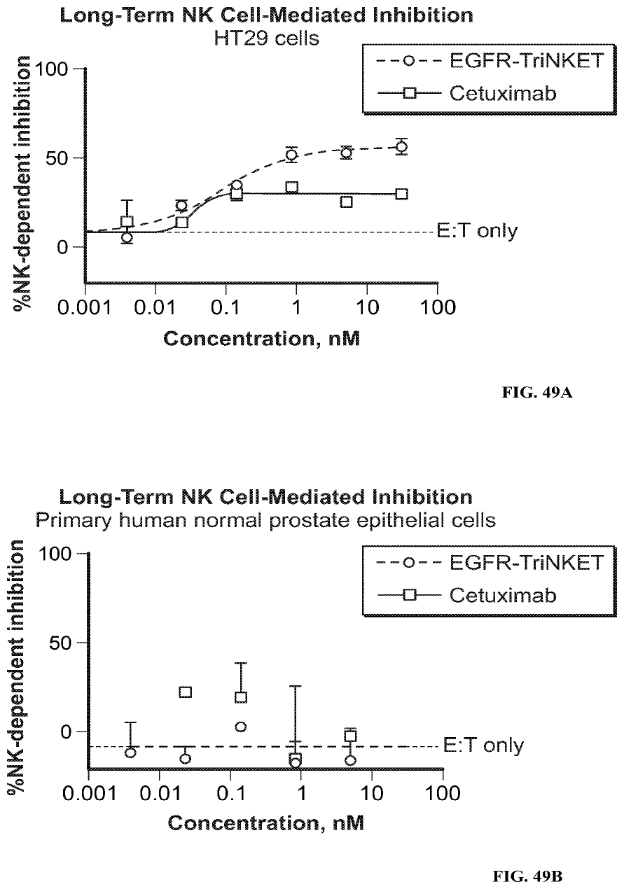

The ability of EGFR-TriNKET to impact the growth of human primary keratinocytes was evaluated against that of cetuximab. EGFR-TriNKET or cetuximab was added to wells pre-seeded with 3000 keratinocytes/well for dose titrations starting at 1000 nM in 1:5 dilutions. Phase images were taken to assess the growth and survival of keratinocytes over time using an IncuCyte® Live-Cell Imager. % Growth inhibition at 72 hours was calculated by comparing masked confluence in treatment wells to no treatment wells, after normalization to the initial scan to control for variability in cell seeding in the well imaging area. As shown in FIG. 48, cetuximab potently inhibited keratinocyte growth (EC₅₀=0.891 nM), while EGFR-TriNKET had at least a 60-fold greater EC₅₀ than cetuximab.

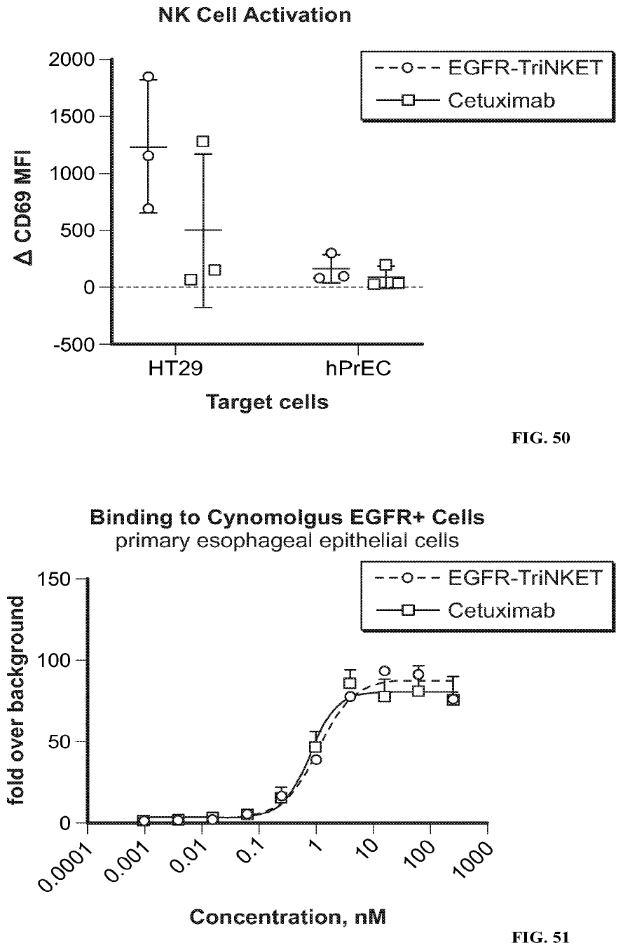

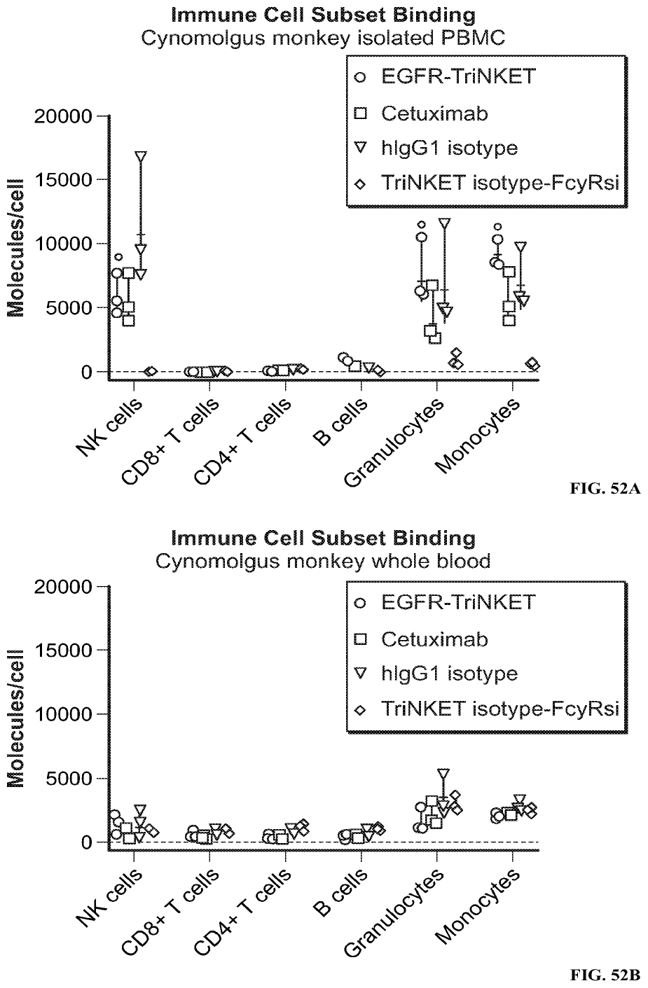

To investigate whether EGFR-TriNKET binding to EGFR expressed on healthy normal cells could stimulate NK cells, primary NK cells from 3 healthy human donors were co-cultured with EGFR⁺ normal prostate epithelial cells (PrECs). EGFR-TriNKET or cetuximab were dose titrated from 30 nM in 1:6 dilutions. After a 72-hour incubation, % NK cell-dependent inhibition was approximated by comparing live target cell counts from sample wells to target cell-only control wells, and additional normalization to treatment wells without NK cells to account for NK cell-independent effects of EGFR-TriNKET or cetuximab. Sensitivity of both cell types to lytic activity by NK cells was confirmed by use of phorbol 12-myristate 13-acetate (PMA)/ionomycin to dysregulate NK cells. Unlike for tumor cell line HT29, against which significant NK cell-mediated lysis (p=0.0001) was observed with the same NK cell donors, EGFR-TriNKET did not trigger significant lysis of PrECs (p=0.6906; FIGS. 49A-49B; Table 24). In addition, CD69 upregulation on NK cells cultured with different epidermal growth factor receptor-expressing (EGFR⁺) target cells (HT29 colorectal adenocarcinoma, and primary human normal prostate epithelial cells [PrECs]) is summarized across 3 human NK cell donors. Overnight-rested primary human NK cells were added to wells overnight pre-seeded with 5000 target cells/well for a 5:1 effector-to-target (E:T) ratio. EGFR-TriNKET and cetuximab were tested at 30 nM and compared to E:T no treatment wells to determine difference (A) in CD69 median fluorescence intensity (MFI). While EGFR-TriNKET induced significant upregulation of NK cell activation marker CD69 when NK cells were co-cultured with HT29 tumor cells (p=0.0004), there was no NK cell activation in the presence of PrECs (p=0.9256; FIG. 50). Cetuximab similarly spared the PrECs but was also less potent at triggering NK-mediated cytolysis when compared to EGFR-TriNKET in the context of HT29 tumor cells. As EGFR expression was greater than 3-fold higher on PrECs than on HT29 cells, the activity and safety profile of EGFR-TRINKET in these studies is likely a function of selective NK cell activation upon engagement when bound to EGFR on tumor cells rather than normal healthy cells.

TABLE 24
Summary of NK Cell-Mediated Cytotoxicity of EGFR-TriNKET on EGFR⁺ Tumor Cell Lines

| | | | EGFR-TriNKET | | Cetuximab | |
|---|---|---|---|---|---|---|
| | | | | Enhanced | | Enhanced |
| | | | | Max NK- | | Max NK- |
| | | | | Dependent | | Dependent |
| | | Average EGFR | EC₅₀ | Inhibition | EC₅₀ | Inhibition |
| Cell Line | Origin; Details | Per Cell | (nM) | (%) | (nM) | (%) |
| Tumor | CRC; EGFRwt, KRASwt | 85,000 | 0.063 ± 0.042 | 46 ± 10 | 0.022 ± 0.014 | 25 ± 16 |
| (HT29) | | | | | | |
| Normal | Normal | 290,000 | ND | ND | ND | ND |
| (PrEC) | prostateepithelial; | | | | | |
| | EGFRwt | | | | | |

### In Vitro Species Cross-Reactivity Assessment of EGFR-TriNKET in Support of Toxicology Studies

The in vitro activity of EGFR-TriNKET in binding EGFR and NKG2D across mice, rats, dogs, and monkeys was analyzed to evaluate the appropriate species for use in toxicology studies. The cynomolgus monkey (Macaca fascicularis) was selected as the only pharmacologically relevant species for the conduct of nonclinical safety studies based on:
The high identity of the amino acid sequences between human and cynomolgus orthologs of both EGFR and NKG2D extracellular domains (ECDs), as well as the similar binding affinity of EGFR-TriNKET for both the human and cynomolgus proteins.
Similar binding profile of EGFR-TriNKET to cynomolgus immune cells compared to the binding profile in human immune cells.
Comparable functional engagement of cynomolgus immune cells via NKG2D and FcγRs, as evaluated by immune subset frequencies, NK cell activation and/or degranulation, and cytokine production in whole blood and/or PBMCs.

### Sequence Comparison of EGFR Orthologs

To identify an appropriate animal species for the toxicological evaluation of EGFR-TriNKET, the EGFR protein sequences from different animal species were compared. EGFR-TriNKET binds to the ECD; therefore, for sequence comparison, the ECD sequence of human EGFR was aligned with the corresponding reference sequences of other species (Table 25). The EGFR ECD sequences are highly conserved between humans and monkeys (Macaca fascicularis and Macaca mulatta), with ˜99% amino acid sequence identity. There are only 8 amino acid differences between humans and monkeys for EGFR.

TABLE 25
Amino Acid Sequence Identity of the EGFR Extracellular
Domain Between Humans and Other Species

| | | | Percent |
|---|---|---|---|
| | | GenBank ® | Identity |
| | | Accession | to Human |
| Species | Common Name | Number | Sequence |
| Homo sapiens | Human | NP_005219.2 | 100% |
| Macaca fascicularis | Cynomolgus monkey | XP_005549616.1 | 98.7% |
| Macaca mulatta | Rhesus monkey | XP_014988922.2 | 98.7% |
| Canis familiaris | Dog | XP_038279602.1 | 89.9% |
| Rattus norvegicus | Rat | NP_113695.2 | 88.9% |
| Mus musculus | Mouse | NP_997538.1 | 88.4% |

Binding Affinity to EGFR, NKG2D, CD16, and FcRn from Different Species

The binding kinetics and affinity of EGFR-TriNKET for purified recombinant human, cynomolgus monkey, rat, and mouse EGFR or NKG2D were obtained using SPR. Measurements were performed at physiological temperature of 37° C. using a Biacore8K SPR system. Commercially available cetuximab and panitumumab were used as EGFR-binding controls.

EGFR-TriNKET bound human and cynomolgus monkey EGFR with comparable kinetic parameters and overall affinity of 4.7 and 5.9 nM, respectively (Table 26). EGFR-TriNKET did not bind to mouse or rat EGFR at the concentrations tested. Like EGFR-TriNKET, cetuximab and panitumumab bound cynomolgus EGFR with kinetic parameters and affinity values that were comparable to the ones for binding human EGFR.

TABLE 26
Kinetics and Affinity of EGFR-TriNKET Binding to
EGFR From Different Species Evaluated by SPR at 37° C.

| Test Article | Target | kₐ (1/Ms) | k_{d} (1/s) | K_{D} (nM) |
|---|---|---|---|---|
| EGFR- | Human EGFR | (2.0 ± 0.0) × 10⁵ | (9.5 ± 0.0) × 10⁻⁴ | 4.7 ± 0.1 |
| TriNKET | Cynomolgus | (1.8 ± 0.1) × 10⁵ | (1.0 ± 0.0) × 10⁻³ | 5.9 ± 0.4 |
| | monkey EGFR | | | |
| | Rat EGFR | No quantifiable binding at the concentrations tested | | |
| | Mouse EGFR | No quantifiable binding at the concentrations tested | | |
| Cetuximab | Human EGFR | (1.6 ± 0.0) × 10⁶ | (5.6 ± 0.1) × 10⁻³ | 4.0 ± 0.2 |
| | Cynomolgus | (1.3 ± 0.1) × 10⁶ | (6.6 ± 0.1) × 10⁻³ | 5.3 ± 0.4 |
| | monkey EGFR | | | |
| | Rat EGFR | No quantifiable binding at the concentrations tested | | |
| | Mouse EGFR | No quantifiable binding at the concentrations tested | | |
| Panitumumab | Human EGFR | (6.2 ± 0.2) × 10⁵ | (6.1 ± 0.2) × 10⁻⁴ | 1.0 ± 0.0 |
| | Cynomolgus | (5.2 ± 0.3) × 10⁵ | (6.7 ± 0.1) × 10⁻⁴ | 1.3 ± 0.1 |
| | monkey EGFR | | | |

| | |
|---|---|
| Rat EGFR | No quantifiable binding at the concentrations tested |
| Mouse EGFR | No quantifiable binding at the concentrations tested |

EGFR-TriNKET comparably bound human and cynomolgus monkey NKG2D with a K_{D} of 481.5 and 561.7 nM, respectively, whereas EGFR-TriNKET did not bind to mouse, rat, or dog NKG2D (Table 27).

TABLE 27
Affinity of EGFR-TriNKET Binding to NKG2D
From Different Species Obtained by SPR

| Test Article | Target | K_{D} (nM) |
|---|---|---|
| EGFR- | Human NKG2D | 481.5 ± 1.5 |
| TriNKET | Cynomolgus | 561.7 ± 29.9 |
| | monkey NKG2D | |
| | Dog NKG2D | No quantifiable binding^{a} |
| | Rat NKG2D | No quantifiable binding^{a} |
| | Mouse NKG2D | No quantifiable bindingª |

SPR assessment of EGFR-TriNKET binding to cynomolgus monkey CD16a (also known as FcγRIIIa), the third modality necessary for the proposed mechanism of action, revealed that EGFR-TriNKET binds the receptor with an affinity comparable to the human IgG1 isotype controls, trastuzumab (K_{D}=80.9±3.0 nM and 71.7±2.7 nM, respectively). Both Kd values were also comparable to the corresponding affinity values obtained for binding human CD16 V158, presented in (K_{D}=53.8±2.0 nM and 48.2±1.4 nM; Table 15).

Similarly, SPR assessment of EGFR-TriNKET binding to cynomolgus monkey FcRn at pH 6.0 revealed similar binding affinity to the human IgG1 isotype control trastuzumab (K_{D}=0.9±0.0 pM and 1.0±0.1 pM, respectively; data on file). The values were also similar to the affinity of both molecules to human FcRn at pH 6.0 (K_{D}=1.0±0.1 pM for both; Table 15). In this experiment, EGFR-TriNKET, as well as trastuzumab, did not have any meaningful binding to cynomolgus FcRn at pH 7.4.

In addition, the dose-responsive binding profile of EGFR-TriNKET to cynomolgus monkey EGFR-expressing cells was compared to that of human EGFR-expressing cells. Both EGFR-TriNKET and cetuximab were dose titrated on cynomolgus monkey EGFR-expressing cells and prepared for detection via flow cytometry using an anti-human Fc secondary conjugated with PE. Test articles were dose titrated from 250 nM in 1:4 dilutions and detected via an anti-human Fc secondary antibody. As shown in FIG. 51, EGFR-TriNKET demonstrated high-affinity binding to cynomolgus monkey EGFR-expressing cells. The EGFR-TriNKET binding EC₅₀ was 1.086 nM for cynomolgus monkey EGFR-expressing cells and was within the range (0.33 to 11 nM) seen for human EGFR-expressing tumor cell lines (Table 14), demonstrating that EGFR-TriNKET binds cynomolgus monkey EGFR-expressing cells with a similar affinity as that for human EGFR-expressing cells.

### Binding Characteristics of EGFR-TriNKET to Cynomolgus Immune Cells

The binding profile of EGFR-TriNKET to cynomolgus monkey NK cells and other immune subsets was assessed by flow cytometry in both cynomolgus whole blood and purified PBMCs using fluorophore-conjugated EGFR-TriNKET. Similar to the binding profile of EGFR-TriNKET in human immune cells, EGFR-TriNKET binding was detected on cynomolgus monkey NK cells, monocytes, and granulocytes within isolated PBMCs (FIGS. 52A-52B (FIG. 52A shows binding to immune cells in isolated PBMCs; FIG. 52B shows binding to immune cells in whole blood)). Binding of EGFR-TriNKET, cetuximab, an hIgG1 isotype control, and a TriNKET isotype control with FcγR-silencing mutations (TriNKET isotype-FcγRsi) to immune cell subsets in either isolated PBMCs or whole blood was assessed across 3 different cynomolgus monkey samples. The same 3 monkeys were used for the PBMC and whole blood studies. Test articles, which were previously directly conjugated with Alexa Fluor® 647 (AF647) for detection by flow cytometry, were introduced at 450 nM. The amount of each molecule bound per cell was calculated by comparing sample median fluorescence intensity signals to those of calibration beads coated with known amounts of AF647 and correcting for the AF647 degree of labeling for each test article. The binding profile of EGFR-TriNKET was similar to that of cetuximab and an isotype control antibody and reflects a profile consistent with FcγR expression of different immune cell subsets. In cynomolgus monkey whole blood, EGFR-TriNKET did not demonstrate significantly greater binding than an isotype control TriNKET with mutations introduced to abrogate FcγR binding (TriNKET isotype-FcγRsi) to FcγR⁺ cells (NK cells, monocytes, and granulocytes), analogous to the profile in human whole blood, likely because of the presence of serum IgG competing for FcγR binding. Overall, the EGFR-TriNKET binding profile in both whole blood and PBMCs was comparable to that of cetuximab or a human IgG1 isotype control for immune cell subsets.

### In Vitro Functional Assessment of Cynomolgus Monkeys as a Relevant Toxicology Species

Cynomolgus monkeys (Macaca fascicularis) were used in the toxicological evaluation of cetuximab and panitumumab, and the adverse reactions observed in monkeys were similar to those observed in humans (Bugelski et al., (2012) Br J Pharmacol. 166(3):823-846), indicating that the cynomolgus monkey is an appropriate animal species for safety testing of these antibodies.

The reactivity of EGFR-TriNKET to monkey antigen was evaluated using SPR and flow cytometry. As shown herein, EGFR-TriNKET bound to human and monkey cells with similar affinities. To demonstrate relevance of this species in assessing the tolerability of EGFR-TriNKET, EGFR-TriNKET was also characterized for functional engagement of cynomolgus immune cells via NKG2D and FcγRs.

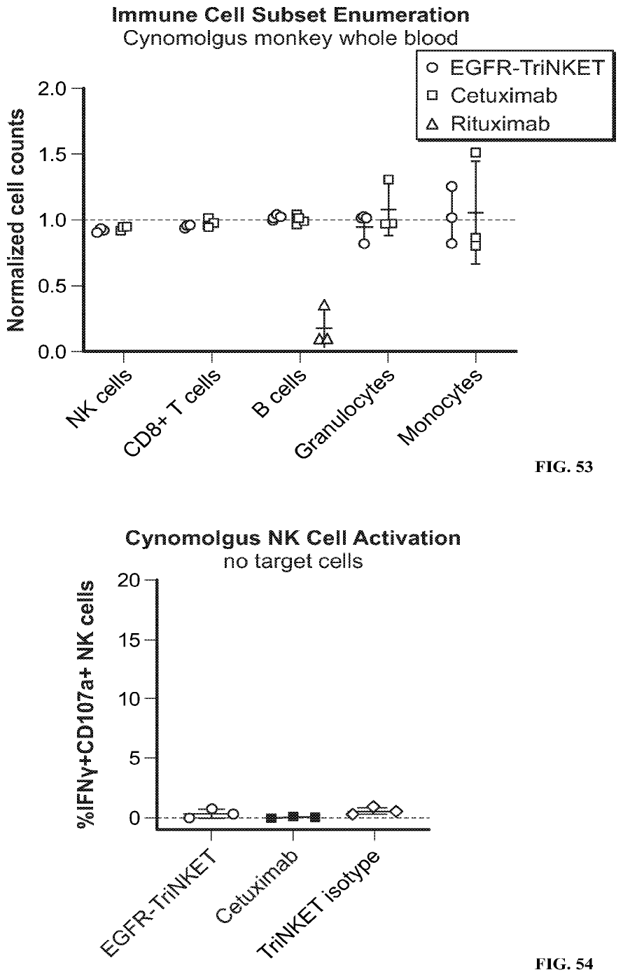

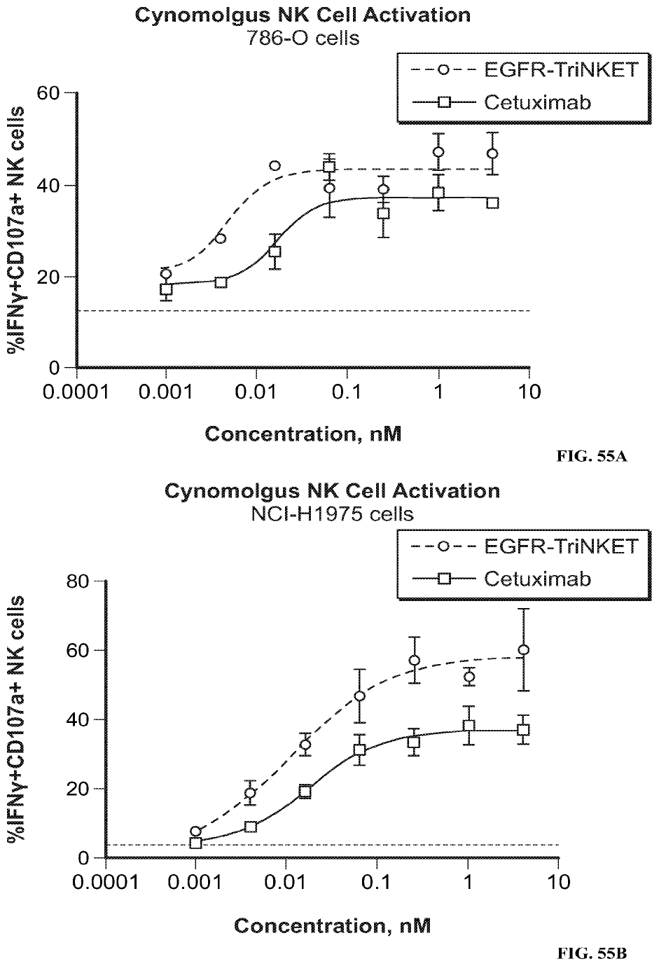

To determine the impact of EGFR-TriNKET on immune cell composition, cynomolgus whole blood was incubated for 24 hours with EGFR-TriNKET or cetuximab, and cell subpopulations (B cells, NK cells, CD8⁺ T cells, monocytes, granulocytes) were subsequently analyzed by FACS (FIG. 53). Cell counts for each of the populations in treated samples were compared to CD4⁺ T-cell counts to control for bulk well technical variation before normalization to untreated control wells. The positive control, rituximab, yielded a specific depletion of 82% for B cells. In contrast, EGFR-TriNKET did not cause substantial changes in any cell subset in any of the samples, similar to the profile of cetuximab. The lack of immune cell depletion observed in cynomolgus whole blood was similar to observations in human whole blood samples (FIG. 44).

As EGFR-TriNKET binding to various cynomolgus monkey immune cells was observed in PBMCs, the influence of EGFR-TriNKET on cynomolgus monkey NK cell activation within PBMCs was examined in the absence of EGFR-expressing target cells. PBMCs were incubated for 4 hours with EGFR-TriNKET, cetuximab, or a TriNKET isotype control, and NK cells were subsequently analyzed by FACS using CD107a and IFNγ accumulation as indicators of NK cell activation (FIG. 54). Brefeldin-A, monensin, and interleukin (IL)-2 were included to increase the dynamic range of detected activation. For CD45⁺CD14−CD20⁻CD3−CD8⁺ NK cells, IFNγ and CD107a gates were drawn based on negative controls and applied to all other samples. Neither EGFR-TriNKET nor cetuximab activated NK cells, and CD107a and IFNγ levels were comparable to those seen in the PBMC sample incubated alone, or with the non-EGFR-targeted TriNKET isotype control.

To determine the functional cross-reactivity of EGFR-TriNKET through cynomolgus NKG2D and CD16a, cynomolgus monkey PBMCs were co-cultured with the human EGFR-expressing cancer cell lines 786-O (kidney adenocarcinoma) and NCI-H1975 (NSCLC adenocarcinoma). Test articles were dose titrated from 4 nM in 1:4 dilutions. Brefeldin-A, monensin, and interleukin (IL)-2 were included to increase the dynamic range of detected activation. For CD45⁺CD14⁻CD20⁻CD3⁻CD8+ NK cells, IFNγ and CD107a gates were drawn based on negative controls and applied to all other samples. As observed with human PBMCs (FIGS. 28A and 28B (FIG. 28A shows degranulation and cytokine production by human NK cells in co-culture with 786-O (renal cell carcinoma) target cells at a 4:1 effector-to-target (E:T) ratio; FIG. 28B shows degranulation and cytokine production by human NK cells in co-culture with NCI-H1975 target cells at a 2:1 E:T); Table 19), EGFR-TriNKET enhanced CD107a degranulation and IFNγ production of cynomolgus NK cells in the presence of both target cell lines (FIGS. 55A and 55B (FIG. 55A shows degranulation and cytokine production by cynomolgus monkey NK cells co-cultured with 786-O cells at a 4:1 E:T ratio; FIG. 55B shows degranulation and cytokine production by cynomolgus monkey NK cells co-cultured with NCI-H1975 cells at a 2:1 E:T. Each point and error bars represent mean and SD, respectively)). EGFR-TriNKET demonstrated similar potency with NK cells across cynomolgus monkeys and humans (Table 28). Greater proportions of both cynomolgus monkey and human NK cells were activated by EGFR-TriNKET than by cetuximab, highlighting the contribution of NKG2D agonism by EGFR-TriNKET for both species. EGFR-TriNKET was designed to require tumor target cross-linking to stimulate robust NK cell activation, and the ability of EGFR-TriNKET to productively engage NKG2D and CD16a on cynomolgus NK cells is tumor target cell-dependent, with similar activity as that observed with human NK cells.

TABLE 28
Comparison of Cynomolgus Monkey and Human NK Cell Degranulation
and Cytokine Production in PBMCs Co-Cultured With Tumor Cells

| | | | Human PBMCs | | Cynomolgus Monkey PBMCs | |
|---|---|---|---|---|---|---|
| | | | | Maximum | | Maximum |
| | | | | IFNγ⁺ | | IFNγ⁺ |
| | | Average EGFR | EC₅₀ | CD107a⁺ | EC₅₀ | CD107a⁺ |
| Cell Line | Origin; Details | Per Cell | (nM) | NKs (%) | (nM) | NKs (%) |
| 786-O | RCC; EGFRwt | 320,000 | 0.089 ± 0.039 | 21 ± 5 | 0.023 ± 0.023 | 31 ± 9 |
| NCI-H1975 | NSCLC-adeno; EGFR | 250,000 | 0.067 ± 0.014 | 35 ± 2 | 0.037 ± 0.023 | 50 ± 9 |
| | mut T790M, L858R | | | | | |

### Pharmacokinetics

The intravenous (IV) PK and toxicokinetic (TK) and immunogenicity profile of EGFR-TriNKET was assessed in a non-GLP toxicology study in cynomolgus monkeys.

### Analytical Methods

In the non-GLP toxicology study, an MSD bioanalytical method was developed using monkey plasma as the matrix for detecting EGFR-TriNKET. The plate was treated with an anti-human IgG and then incubated with EGFR-TriNKET reference standards or cynomolgus monkey plasma samples. The primary detection antibody was a biotinylated His-Tag human EGFR antibody, and the secondary detection antibody was a streptavidin-conjugated Sulfo-Tag antibody. The lower limit of quantitation (LLOQ) was 50.0 μg/mL, and the upper limit of quantitation (ULOQ) was 20,000 μg/mL.

Also in the non-GLP toxicology study, immunogenicity was assessed using a procedure for the detection of anti-EGFR-TriNKET antibodies in monkey serum.

### Pharmacokinetic Properties of EGFR-TriNKET

The PK and TK profile of EGFR-TriNKET was investigated after repeat weekly IV administration (e.g., about 1, about 10, about 50 mg/kg) up to 5 doses in cynomolgus monkeys. There did not appear to be an overall sex-related difference in the PK of EGFR-TriNKET after weekly IV administration (Table 29).

Maximum concentration (C) in plasma increased approximately proportionately with increasing dose over the dose range of 1 to 50 mg/kg. The area under the concentration-time curve from 0 to 144 hours post-dose (AUC₀₋₁₄₄) increased in a greater than dose-proportional manner over the dose range of 1 to 50 mg/kg on Day 1 (approximately 2.9-fold higher than those values predicted from a linear relationship). After repeated IV infusion doses (Day 22), the C and AUC₀₋₁₄₄ of EGFR-TriNKET suggested that some accumulation (approximately 1.7-fold) occurred at these dose levels. Anti-drug antibody (ADA) production was detected in animals at the 2 lower dose levels (e.g., about 1 and about 10 mg/kg); therefore, it was not possible to make a reliable assessment of dose proportionality after repeated dosing. EGFR-TriNKET declined fairly slowly after the 30-minute IV infusion, with a long terminal half-life, generally in excess of 80 hours. Cetuximab and panitumumab are known to demonstrate target-mediated drug disposition (TMDD) in monkeys and humans at dose levels generally lower than ˜2 mg/kg and related to binding the ubiquitous EGFR target.

TABLE 29
Pharmacokinetic Assessment of EGFR-TriNKET in Plasma (Study TG57CQ)

| Dose | Cmax (μg/mL) | | | | AUC₀₋₁₄₄ (h*μg/mL) | | | | t_{1/2} (h) | | | |
|---|---|---|---|---|---|---|---|---|---|---|---|---|
| Level | Day 1 | | Day 22 | | Day 1 | | Day 22 | | Day 1 | | Day 22 | |
| (mg/kg) | M | F | M | F | M | F | M | F | M | F | M | F |
| 1 | 32.0 | 36.0 | 6.94^{a} | 17.8^{a} | 635 | 775 | 11.4^{a} | 127^{a} | 87^{b} | 49^{b} | 1.0 | 11 |
| 10 | 285^{a} | 329 | 413^{a} | 299^{a} | 15,700^{a} | 17,400 | 25,700^{a} | 7530^{a} | 100^{b} | 86^{b} | 160^{b} | 17 |
| 50 | 1400 | 1540 | 2060 | 2060 | 98,400 | 109,000 | 169,000 | 175,000 | 97^{b} | 100^{b} | 140^{b} | 100^{b} |

### Example 4: Purification of EGFR-TriNKET

The exemplary downstream purification process for EGFR-TriNKET, incorporating: (a) a first polypeptide with the amino acid sequence of SEQ ID NO:167; (b) a second polypeptide with the amino acid sequence of SEQ ID NO:164; and (c) a third polypeptide with the amino acid sequence of SEQ ID NO:165, was developed through a series of small-scale purification runs using clarified harvest from an upstream material supply run. The exemplary purification process for EGFR-TriNKET is summarized in this example which includes parameters for chromatography, viral inactivation, viral filtration, and UF/DF operations. Process buffer descriptions, recipes, and specifications are also included.

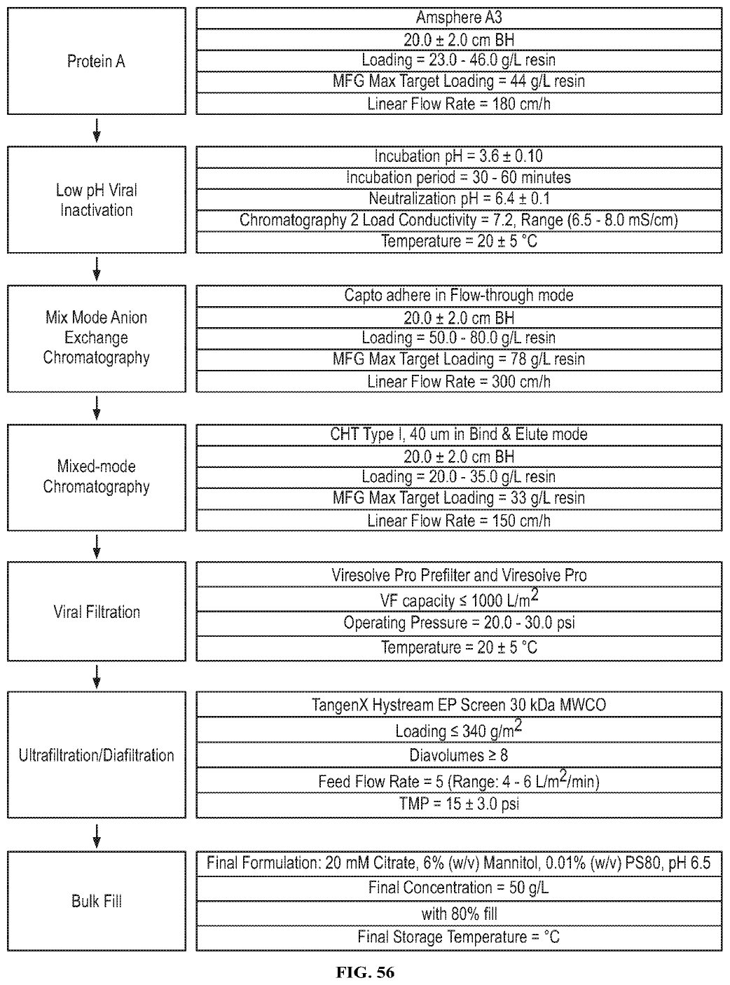

The exemplary purification process for EGFR-TriNKET (e.g., incorporating: (a) a first polypeptide with the amino acid sequence of SEQ ID NO:167; (b) a second polypeptide with the amino acid sequence of SEQ ID NO:164; and (c) a third polypeptide with the amino acid sequence of SEQ ID NO:165) is summarized in FIG. 56 and involves a capture step using Amsphere A3 chromatography resin followed by a low pH viral inactivation. Post viral inactivation and neutralization, the VIN eluate is filtered using a 3M Zeta Plus 90ZB08A filter. Capto adhere and CHT Type I, 40 um polishing chromatography unit operations are performed after to reduce impurities. Viral filtration is performed using a Millipore Viresolve Pro Prefilter pre-filter and a Millipore Viresolve Pro virus filter. The product stream is then concentrated and buffer exchanged into 20 mM Citrate, pH 6.5 using a TangenX Hystream 30 kDa EP Screen membrane (Repligen). The product is then spiked with 20 mM citrate, 12% (w/v) mannitol, 0.02% PS80, pH 6.5 to achieve a formulation composition of 20 mM citrate, 6% (w/v) mannitol, 0.01% PS80, pH 6.5 and filtered through a 0.2 μm PES filter and transferred to its final storage conditions.

A 0.2 μm PES pre-column guard filter is used for each chromatography operation.

### Amsphere A3 Chromatography

The first unit operation in the downstream process is Protein A chromatography using Amsphere A3 resin. Prior to loading, the chromatography column is sanitized with 0.1 M NaOH; after 3 CVs, the column is held for 15 minutes before equilibration. Clarified harvest is loaded neat onto the chromatography column. A wash using 20 mM Tris, 150 mM NaCl, pH 7.5 is performed after loading to flush the product off the column and remove any impurities nonspecifically bound to the Protein A resin. A second post load wash using 50 mM acetate, pH 5.4 is performed to further remove any bound impurities. The product is eluted off the column using 50 mM acetate, pH 3.7. Product collection begins at an A280 greater than or equal to 1 AU/cm and ends at an A280 less than or equal to 1 AU/cm. The neat Protein A eluate is stable up to 3 days at 15-25° C. and 7 days at 2-8° C. Once all cycles have been completed, the neat Protein A eluate pool is forward processed through the low pH viral inactivation.

### Low pH Viral Inactivation

Prior to the start of viral inactivation, the Protein A eluates are pooled together. Viral inactivation is initiated by titrating the product to 3.6±0.10 with 2 M Acetic Acid. The product was held at room temperature for 30-60 minutes. No mixing occurs during the low pH hold time. To end the incubation period, 2 M Tris is used to titrate the product to 6.4±0.1. The viral-inactivated and neutralized (VIN) eluate is then filtered using a Zeta Plus 90ZB08A filter and a Sartopore 2 filter. The typical yield for viral inactivation and depth filtration is 95±5%. Pool is stable for 3 days at 15-25° C. and 7 days at 2-8° C.

### Capto Adhere Chromatography

The next unit operation in the downstream process is Capto adhere chromatography operated in flow-through mode. The column is equilibrated with at least ≥5 CVs of 10 mM Phosphate, 65 mM NaCl, pH 6.4. After equilibration, the load is passed through the column with product collection starting at 0.5 AU/cm and ends collection at less than or equal to 0.5 AU/cm. The Capto adhere load material is generated by spiking the VIN eluate pool with 1.3% w/w of 400 mM phosphate, pH 6.4. The pool is then spiked with 2 M NaCl to target a conductivity of 6.5-8.0 mS/cm and then titrated with 2M acetic acid or 2M Tris base to pH 6.4±0.1 if out of range. The Capto adhere flow-through is stable up to 3 days at 15-25° C. and 7 days at 2-8° C.

### CHT Type I, 40 μm Chromatography

The CHT Type I, 40 μm chromatography step is operated in bind and elute mode. The CHT Type I, 40 μm column is equilibrated for ≥5 CVs using 10 mM Phosphate, 50 mM MES, pH 7.0. After equilibration, the load is passed through the column, followed by a post load wash step. CHT Type I, 40 μm load is generated by titrating Capto adhere flowthrough to pH 7.0 using 2 M Tris Base. A 5CV wash is performed using 10 mM Phosphate, 50 mM MES, pH 7.0. The product is eluted from the column using 8 CVs of 10 mM Phosphate, 50 mM MES, 200 mM NaCl pH 7.0 buffer. CHT Type I, 40 μm eluate is stable up to 3 days at 15-25° C. and 7 days at 2-8° C.

### Viral Filtration

The Millipore Viresolve Pro Prefilter is first flushed with ≥100 L/m² of WFI at a pressure of 10.0-20.0 psig before the Viresolve Pro filter is attached. Once the Viresolve Pro filter is inline, the filter setup is flushed with ≥50 L/m² WFI at 30.0-50.0 psig. Then, the pre-filter and virus filter are flushed with ≥50 L/m² of 20.0-30.0. Product is then filtered at 20.0-30.0 psig using the viral filtration setup. Once loading has finished, the operation may be paused for a maximum of 25 minutes while switching between the load and buffer flush. After pausing, the filter is flushed with 45±5 L/m² of EQ buffer 2 to recover the product remaining in the holdup volume of the filtration assembly. Viral filtrate is stable up to 3 days at 15-25° C. and 7 days at 2-8° C.

### Ultrafiltration/Diafiltration

An ultrafiltration (UF)/diafiltration (DF) unit operation is required to buffer exchange and concentrate the final product EGFR-TriNKET to a final product concentration of 100 g/L. A sanitized TangenX Hystream 30 kDa MWCO cassette is flushed with ≥20 L/m² WFI followed by ≥20 L/m² of 20 mM Citrate, pH 6.5. The viral filtrate is then loaded to a max of 340 g/m² and concentrated to 25 g/L prior to being diafiltered against 20 mM Citrate, pH 6.5 for at least 8 diavolumes. After diafiltration is completed, product is recovered from the system, which is described in the following section. Retentate is stable up to 3 days at 15-25° C. and 7 days at 2-8° C.

### Bulk Fill

The final unit operation in the downstream purification process is bulk fill. The buffer exchanged final product is 0.2 μm filter into the final storage containers. The final formulation for this molecule is 20 mM citrate, 6% (w/v) Mannitol, 0.01% (w/v) Polysorbate 80, pH 6.5.

### Example 5: Treatment of EGFR-TriNKET in Human Subjects

An EGFR-TriNKET composition comprising EGFR-TriNKET, incorporating: (a) a first polypeptide with the amino acid sequence of SEQ ID NO:167; (b) a second polypeptide with the amino acid sequence of SEQ ID NO:164; and (c) a third polypeptide with the amino acid sequence of SEQ ID NO:165, was administered to human subjects.

Three human subjects who had advanced (unresectable, recurrent, or metastatic) solid tumors were selected. These patients had tumor types of epithelial origin for which the expression of EGFR was known. No effective standard therapy existed for these patients, they had recurrent disease, or they were intolerant of standard therapies.

These patients were dosed weekly with the EGFR-TriNKET composition by intravenous infusion at a dose of 0.1 mg/kg. As of the administration of the seventh dose, no treatment-related adverse events had been observed.

### INCORPORATION BY REFERENCE

Unless stated to the contrary, the entire disclosure of each of the patent documents and scientific articles referred to herein is incorporated by reference for all purposes.

### EQUIVALENTS

The present application may be embodied in other specific forms without departing from the spirit or essential characteristics thereof. The foregoing embodiments are therefore to be considered in all respects illustrative rather than limiting the application described herein. Scope of the present application is thus indicated by the appended claims rather than by the foregoing description, and all changes that come within the meaning and range of equivalency of the claims are intended to be embraced therein.

What is claimed is:

1. A pharmaceutical formulation comprising:
(a) a multi-specific binding protein comprising:
(i) a Fab comprising a heavy chain variable domain (VH) and a light chain variable domain (VL) that bind NKG2D;
(ii) a single-chain variable fragment (scFv) comprising a VH and a VL that bind EGFR; and
(iii) an antibody Fc domain, and
(b) one or more of:
(i) 15 mM to 25 mM citrate; and
(ii) 4% to 8% (w/v) mannitol,
at pH 6.0 to 7.0.

2. The pharmaceutical formulation of claim 1, further comprising a polysorbate.

3. A pharmaceutical formulation comprising:
(a) a multi-specific binding protein comprising:
(i) a Fab comprising a heavy chain variable domain (VH) and a light chain variable domain (VL) that bind NKG2D;
(ii) a single-chain variable fragment (scFv) that binds EGFR, wherein the scFv comprises:
1) a VH having complementarity-determining region 1 (CDR1), complementarity-determining region 2 (CDR2), and complementarity-determining region 3 (CDR3) sequences of SEQ ID NOs: 136, 157, and 138, respectively; and a VL having CDR1, CDR2, and CDR3 sequences of SEQ ID NOs: 140, 141, and 151, respectively; or
2) a VH having CDR1, CDR2, and CDR3 sequences of SEQ ID NOs: 136, 146, and 138, respectively; and a VL having CDR1, CDR2, and CDR3 sequences of SEQ ID NOs: 140, 141, and 142, respectively; and
(iii) an antibody Fc domain;
(b) citrate;
(c) a sugar or sugar alcohol; and
(d) a polysorbate,
at pH 6.0 to 7.0.

4. The pharmaceutical formulation of any one of claims 1 to 3, wherein the concentration of the multi-specific binding protein in the pharmaceutical formulation is 1 mg/mL to 125 mg/mL.

5. The pharmaceutical formulation of any one of claims 1 to 4, wherein the concentration of the multi-specific binding protein in the pharmaceutical formulation is 2 mg/mL to 100 mg/mL.

6. The pharmaceutical formulation of any one of claims 1 to 5, wherein the concentration of the multi-specific binding protein in the pharmaceutical formulation is 5 mg/mL to 50 mg/mL.

7. The pharmaceutical formulation of any one of claims 1 to 6, wherein the concentration of the multi-specific binding protein in the pharmaceutical formulation is 7.5 mg/mL to 25 mg/mL.

8. The pharmaceutical formulation of any one of claims 1 to 7, wherein the concentration of the multi-specific binding protein in the pharmaceutical formulation is 10 mg/mL to 20 mg/mL.

9. The pharmaceutical formulation of any one of claims 1 to 8, wherein the concentration of the multi-specific binding protein in the pharmaceutical formulation is about 15 mg/mL.

10. The pharmaceutical formulation of any one of claims 1 to 9, wherein the formulation is diluted with a suitable diluent in the range of 1:0 to 1:10 prior to administration to a subject.

11. The pharmaceutical formulation of any one of claims 1 to 10, wherein the pharmaceutical formulation comprises 15 mM to 25 mM citrate.

12. The pharmaceutical formulation of any one of claims 1 to 11, wherein the pharmaceutical formulation comprises 17.5 mM to 22.5 mM citrate.

13. The pharmaceutical formulation of any one of claims 1 to 12, wherein the pharmaceutical formulation comprises about 20 mM citrate.

14. The pharmaceutical formulation of any one of claims 3 to 13, wherein the sugar alcohol is an alcohol of a monosaccharide.

15. The pharmaceutical formulation of any one of claims 3 to 14, wherein the sugar alcohol is mannitol.

16. The pharmaceutical formulation of any one of claims 1, 2, or 15, wherein the pharmaceutical formulation comprises 4% to 8% (w/v) mannitol.

17. The pharmaceutical formulation of any one of claims 1, 2, 15 or 16, wherein the pharmaceutical formulation comprises 5% to 7% (w/v) mannitol.

18. The pharmaceutical formulation of any one of claims 1, 2, or 15 to 17, wherein the pharmaceutical formulation comprises about 6% (w/v) mannitol.

19. The pharmaceutical formulation of any one of claims 2 to 18, wherein the polysorbate is polysorbate 80.

20. The pharmaceutical formulation of claim 19, wherein the pharmaceutical formulation comprises 0.005% to 0.05% (w/v) polysorbate 80.

21. The pharmaceutical formulation of claim 19 or 20, wherein the concentration of polysorbate 80 is 0.0075% to 0.025% (w/v).

22. The pharmaceutical formulation of any one of claims 19 to 21, wherein the concentration of polysorbate 80 is about 0.01% (w/v).

23. The pharmaceutical formulation of any one of claims 1 to 22, wherein the pH is 6.2 to 6.8.

24. The pharmaceutical formulation of any one of claims 1 to 23, wherein the pH is 6.4 to 6.6.

25. The pharmaceutical formulation of any one of claims 1 to 24, wherein the pH is about 6.5.

26. The pharmaceutical formulation of any one of claims 1 to 6, 10, 11, 14 to 16, 19, 20, or 23, wherein the formulation comprises:
(a) 5 mg/mL to 50 mg/mL of the multi-specific binding protein;
(b) 15 mM to 25 mM citrate;
(c) 4% to 8% (w/v) mannitol; and
(d) 0.005% to 0.05% (w/v) polysorbate 80,
at pH 6.2 to 6.8.

27. The pharmaceutical formulation of any one of claims 1 to 8, 10 to 12, 14 to 17, 19 to 21, 23, or 24, wherein the formulation comprises:
(a) 10 mg/mL to 20 mg/mL of the multi-specific binding protein;
(b) 17.5 mM to 22.5 mM citrate;
(c) 5% to 7% (w/v) mannitol; and
(d) 0.0075% to 0.025% (w/v) polysorbate 80,
at pH 6.4 to 6.6.

28. The pharmaceutical formulation of any one of claims 1 to 27, wherein the formulation comprises:
(a) about 15 mg/mL of the multi-specific binding protein;
(b) about 20 mM citrate;
(c) about 6% (w/v) mannitol; and
(d) about 0.01% (w/v) polysorbate 80,
at about pH 6.5.

29. The pharmaceutical formulation of any one of claims 1 to 28, wherein the VL of the scFv is linked to the VH of the scFv via a flexible linker.

30. The pharmaceutical formulation of claim 29, wherein the flexible linker comprises the amino acid sequence of SEQ ID NO:119.

31. The pharmaceutical formulation of claim 29 or 30, wherein the flexible linker consists of the amino acid sequence of SEQ ID NO:119.

32. The pharmaceutical formulation of any one of claims 1 to 31, wherein the VL of the scFv is positioned to the N-terminus of the VH of the scFv, or the VH of the scFv is positioned to the N-terminus of the VL of the scFv.

33. The pharmaceutical formulation of any one of claims 1 to 32, wherein the VH of the scFv forms a disulfide bridge with the VL of the scFv.

34. The pharmaceutical formulation of claim 33, wherein the disulfide bridge is formed between C44 of the VH of the scFv and C100 of the VL of the scFv, numbered under the Kabat numbering scheme.

35. The pharmaceutical formulation of any one of claims 1 to 34, wherein the antibody Fc domain comprises a first antibody Fc polypeptide linked to the Fab and a second antibody Fc polypeptide linked to the scFv.

36. The pharmaceutical formulation of claim 35, wherein the first antibody Fc polypeptide is linked to a heavy chain portion of the Fab.

37. The pharmaceutical formulation of claim 35 or 36, wherein the scFv is linked to the second antibody Fc polypeptide via a hinge comprising Ala-Ser or Gly-Ser.

38. The pharmaceutical formulation of any one of claims 35 to 37, wherein the first and second antibody Fc polypeptides each comprise a hinge and a CH2 domain of a human IgG1 antibody.

39. The pharmaceutical formulation of claim 38, wherein the first and second antibody Fc polypeptides each comprise an amino acid sequence at least 90% identical to amino acids 234-332 of a wild-type human IgG1 antibody, numbered according to the EU index.

40. The pharmaceutical formulation of any one of claims 35 to 39, wherein the first and second antibody Fc polypeptides each comprise different mutations promoting heterodimerization.

41. The pharmaceutical formulation of claim 40, wherein the first antibody Fc polypeptide is a human IgG1 Fc polypeptide comprising K360E and K409W substitutions, numbered according to the EU index.

42. The pharmaceutical formulation of claim 40 or 41, wherein the second antibody Fc polypeptide is a human IgG1 Fc polypeptide comprising Q347R, D399V, and F405T substitutions, numbered according to the EU index.

43. The pharmaceutical formulation of any one of claims 1 to 42, wherein the Fab comprises:
(a) a VH comprising CDR1, CDR2, and CDR3 sequences of SEQ ID NOs: 81, 82, and 112, respectively; and
(b) a VL comprising CDR1, CDR2, and CDR3 sequences of SEQ ID NOs: 86, 77, and 87, respectively.

44. The pharmaceutical formulation of any one of claims 1 to 43, wherein the Fab comprises:
(a) a VH comprising CDR1, CDR2, and CDR3 sequences of SEQ ID NOs: 81, 82, and 97, respectively; and
(b) a VL comprising CDR1, CDR2, and CDR3 sequences of SEQ ID NOs: 86, 77, and 87, respectively.

45. The pharmaceutical formulation of any one of claims 1 to 44, wherein the Fab comprises a VH comprising an amino acid sequence at least 90% identical to SEQ ID NO:95 or at least 90% identical to SEQ ID NO:110, and a VL comprising an amino acid sequence at least 90% identical to SEQ ID NO:85.

46. The pharmaceutical formulation of any one of claims 1 to 45, wherein the Fab comprises a VH comprising the amino acid sequence of SEQ ID NO:95, and a VL comprising the amino acid sequence of SEQ ID NO:85.

47. The pharmaceutical formulation of any one of claims 1 to 46, wherein:
a) the VH of the scFv comprises CDR1, CDR2, and CDR3 sequences of SEQ ID NOs: 136, 157, and 138, respectively; and the VL of the scFv comprises CDR1, CDR2, and CDR3 sequences of SEQ ID NOs: 140, 141, and 151, respectively; or
b) the VH of the scFv comprises CDR1, CDR2, and CDR3 sequences of SEQ ID NOs: 136, 146, and 138, respectively; and the VL of the scFv comprises CDR1, CDR2, and CDR3 sequences of SEQ ID NOs: 140, 141, and 142, respectively.

48. The pharmaceutical formulation of any one of claims 1 to 47, wherein the VH of the scFv comprises CDR1, CDR2, and CDR3 sequences of SEQ ID NOs: 136, 157, and 138, respectively; and the VL of the scFv comprises CDR1, CDR2, and CDR3 sequences of SEQ ID NOs: 140, 141, and 151, respectively.

49. The pharmaceutical formulation of any one of claims 1 to 48, wherein the VH of the scFv comprises an amino acid sequence at least 90% identical to SEQ ID NO:156, and the VL of the scFv comprises an amino acid sequence at least 90% identical to SEQ ID NO:150.

50. The pharmaceutical formulation of any one of claims 1 to 49, wherein the scFv comprises the amino acid sequence of SEQ ID NO:158 or SEQ ID NO:159.

51. The pharmaceutical formulation of any one of claims 1 to 46, wherein the VH of the scFv comprises CDR1, CDR2, and CDR3 sequences of SEQ ID NOs: 136, 146, and 138, respectively; and the VL of the scFv comprises CDR1, CDR2, and CDR3 sequences of SEQ ID NOs: 140, 141, and 151, respectively.

52. The pharmaceutical formulation of any one of claims 1 to 46, or 51, wherein the VH of the scFv comprises an amino acid sequence at least 90% identical to SEQ ID NO:170, and the VL of the scFv comprises an amino acid sequence at least 90% identical to SEQ ID NO:171.

53. The pharmaceutical formulation of any one of claims 1 to 46, 51, or 52, wherein the VH of the scFv comprises the amino acid sequence of SEQ ID NO:170, and the VL of the scFv comprises the amino acid sequence of SEQ ID NO:171.

54. The pharmaceutical formulation of any one of claims 1 to 46, or 51 to 53, wherein the scFv comprises the amino acid sequence of SEQ ID NO:152 or SEQ ID NO:153.

55. The pharmaceutical formulation of any one of claims 1 to 34, wherein
(a) the VH of the Fab comprises CDR1, CDR2, and CDR3 sequences of SEQ ID NOs: 81, 82, and 97, respectively; and the VL of the Fab comprises CDR1, CDR2, and CDR3 sequences of SEQ ID NOs: 86, 77, and 87, respectively;
(b) the VH of the scFv comprises CDR1, CDR2, and CDR3 sequences of SEQ ID NOs: 136, 146, and 138, respectively; and the VL of the scFv comprises CDR1, CDR2, and CDR3 sequences of SEQ ID NOs: 140, 141, and 151, respectively; and
(c) the antibody Fc domain comprises a first antibody Fc polypeptide linked to the Fab and a second antibody Fc polypeptide linked to the scFv, wherein the first antibody Fc polypeptide is a human IgG1 Fc polypeptide comprising K360E and K409W substitutions, and the second antibody Fc polypeptide is a human IgG1 Fc polypeptide comprising Q347R, D399V, and F405T substitutions, numbered according to the EU index.

56. The pharmaceutical formulation of claim 55, wherein
(a) the VH of the Fab comprises the amino acid sequence of SEQ ID NO:95 or SEQ ID NO:110, and the VL of the Fab comprises the amino acid sequence of SEQ ID NO:85; and
(b) the VH of the scFv comprises the amino acid sequence of SEQ ID NO:170, and the VL of the scFv comprises the amino acid sequence of SEQ ID NO:171.

57. The pharmaceutical formulation of claim 56, wherein the scFv comprises the amino acid sequence of SEQ ID NO:152 or SEQ ID NO:153.

58. The pharmaceutical formulation of any one of claims 1 to 57, wherein the multi-specific binding protein comprises:
(a) a first polypeptide comprising the amino acid sequence of SEQ ID NO:167;
(b) a second polypeptide comprising the amino acid sequence of SEQ ID NO:164; and
(c) a third polypeptide comprising the amino acid sequence of SEQ ID NO:165.

59. The pharmaceutical formulation of any one of claims 51 to 58, comprising:
(a) about 15 mg/mL of the multi-specific binding protein;
(b) about 20 mM citrate;
(c) about 6% (w/v) mannitol; and
(d) about 0.01% (w/v) polysorbate 80,
at about pH 6.5.

60. The pharmaceutical formulation of any one of claims 1 to 46, wherein the VH of the scFv comprises CDR1, CDR2, and CDR3 sequences of SEQ ID NOs: 136, 137, and 138, respectively; and the VL of the scFv comprises CDR1, CDR2, and CDR3 sequences of SEQ ID NOs: 140, 141, and 151, respectively.

61. The pharmaceutical formulation of any one of claims 1 to 46, or 60, wherein the VH of the scFv comprises an amino acid sequence at least 90% identical to SEQ ID NO:135, and the VL of the scFv comprises an amino acid sequence at least 90% identical to SEQ ID NO:150.

62. The pharmaceutical formulation of any one of claims 1 to 46, 60, or 61, wherein the VH of the scFv comprises the amino acid sequence of SEQ ID NO:135, wherein the glycine at position 44 of the VH is substituted with a cysteine (C44), numbered under the Kabat numbering scheme, and the VL of the scFv comprises the amino acid sequence of SEQ ID NO:150, wherein the glycine at position 100 of the VL is substituted with a cysteine (C100), numbered under the Kabat numbering scheme, and wherein the C44 and the C100 form a disulfide bond between the VH and the VL.

63. The pharmaceutical formulation of any one of claims 1 to 46, or 60 to 62, wherein the scFv comprises the amino acid sequence of SEQ ID NO:154 or SEQ ID NO:155.

64. The pharmaceutical formulation of any one of claims 1 to 34, wherein
(a) the VH of the Fab comprises CDR1, CDR2, and CDR3 sequences of SEQ ID NOs: 81, 82, and 97, respectively; and the VL of the Fab comprises CDR1, CDR2, and CDR3 sequences of SEQ ID NOs: 86, 77, and 87, respectively;
(b) the VH of the scFv comprises CDR1, CDR2, and CDR3 sequences of SEQ ID NOs: 136, 137, and 138, respectively; and the VL of the scFv comprises CDR1, CDR2, and CDR3 sequences of SEQ ID NOs: 140, 141, and 151, respectively; and
(c) the antibody Fc domain comprises a first antibody Fc polypeptide linked to the Fab and a second antibody Fc polypeptide linked to the scFv, wherein the first antibody Fc polypeptide is a human IgG1 Fc polypeptide comprising K360E and K409W substitutions, and the second antibody Fc polypeptide is a human IgG1 Fc polypeptide comprising Q347R, D399V, and F405T substitutions, numbered according to the EU index.

65. The pharmaceutical formulation of claim 64, wherein
(a) the VH of the Fab comprises the amino acid sequence of SEQ ID NO:95 or SEQ ID NO:110, and the VL of the Fab comprises the amino acid sequence of SEQ ID NO:85; and
(b) the VH of the scFv comprises an amino acid sequence at least 90% identical to the amino acid sequence of SEQ ID NO:135, and the VL of the scFv comprises an amino acid sequence at least 90% identical to the amino acid sequence of SEQ ID NO:150.

66. The pharmaceutical formulation of claim 65, wherein the scFv comprises the amino acid sequence of SEQ ID NO:154 or SEQ ID NO:155.

67. The pharmaceutical formulation of any one of claims 60 to 66, comprising:
(a) about 15 mg/mL of the multi-specific binding protein;
(b) about 20 mM citrate;
(c) about 6% (w/v) mannitol; and
(d) about 0.01% (w/v) polysorbate 80,
at about pH 6.5.

68. The pharmaceutical formulation of any one of claims 1 to 46, wherein the VH of the scFv comprises CDR1, CDR2, and CDR3 sequences of SEQ ID NOs: 136, 146, and 138, respectively; and the VL of the scFv comprises CDR1, CDR2, and CDR3 sequences of SEQ ID NOs: 140, 141, and 142, respectively.

69. The pharmaceutical formulation of any one of claims 1 to 46, or 68, wherein the VH of the scFv comprises an amino acid sequence at least 90% identical to SEQ ID NO:145, and the VL of the scFv comprises an amino acid sequence at least 90% identical to SEQ ID NO:147.

70. The pharmaceutical formulation of any one of claims 1 to 46, 68, or 69, wherein the VH of the scFv comprises the amino acid sequence of SEQ ID NO:145, wherein the glycine at position 44 of the VH is substituted with a cysteine (C44), numbered under the Kabat numbering scheme, and the VL of the scFv comprises the amino acid sequence of SEQ ID NO:147, wherein the glycine at position 100 of the VL is substituted with a cysteine (C100), numbered under the Kabat numbering scheme, and wherein the C44 and the C100 form a disulfide bond between the VH and the VL.

71. The pharmaceutical formulation of any one of claims 1 to 46, or 68 to 70, wherein the scFv comprises the amino acid sequence of SEQ ID NO:148 or SEQ ID NO:149.

72. The pharmaceutical formulation of any one of claims 1 to 34, wherein
(a) the VH of the Fab comprises CDR1, CDR2, and CDR3 sequences of SEQ ID NOs: 81, 82, and 97, respectively; and the VL of the Fab comprises CDR1, CDR2, and CDR3 sequences of SEQ ID NOs: 86, 77, and 87, respectively;
(b) the VH of the scFv comprises CDR1, CDR2, and CDR3 sequences of SEQ ID NOs: 136, 146, and 138, respectively; and the VL of the scFv comprises CDR1, CDR2, and CDR3 sequences of SEQ ID NOs: 140, 141, and 142, respectively; and
(c) the antibody Fc domain comprises a first antibody Fc polypeptide linked to the Fab and a second antibody Fc polypeptide linked to the scFv, wherein the first antibody Fc polypeptide is a human IgG1 Fc polypeptide comprising K360E and K409W substitutions, and the second antibody Fc polypeptide is a human IgG1 Fc polypeptide comprising Q347R, D399V, and F405T substitutions, numbered according to the EU index.

73. The pharmaceutical formulation of claim 72, wherein
(a) the VH of the Fab comprises the amino acid sequence of SEQ ID NO:95 or SEQ ID NO:110, and the VL of the Fab comprises the amino acid sequence of SEQ ID NO:85; and
(b) the VH of the scFv comprises an amino acid sequence at least 90% identical to the amino acid sequence of SEQ ID NO:145, and the VL of the scFv comprises an amino acid sequence at least 90% identical to the amino acid sequence of SEQ ID NO:147.

74. The pharmaceutical formulation of claim 73, wherein the scFv comprises the amino acid sequence of SEQ ID NO:148 or SEQ ID NO:149.

75. The pharmaceutical formulation of any one of claims 68 to 74, comprising:
(a) about 15 mg/mL of the multi-specific binding protein;
(b) about 20 mM citrate;
(c) about 6% (w/v) mannitol; and
(d) about 0.01% (w/v) polysorbate 80,
at about pH 6.5.

76. The pharmaceutical formulation of any one of claims 1 to 75, wherein more than 97% of the multi-specific binding protein has native conformation, as determined by size-exclusion chromatography.

77. The pharmaceutical formulation of any one of claims 1 to 76, wherein less than 2% of the multi-specific binding protein form a high molecular weight complex, as determined by size-exclusion chromatography.

78. The pharmaceutical formulation of any one of claims 1 to 77, wherein the multi-specific binding protein binds human CD16 with a binding affinity (K_{D}) of 48 nM to 160 nM, as measured by surface plasmon resonance (SPR).

79. The pharmaceutical formulation of any one of claims 1 to 78, wherein the multi-specific binding protein binds EGFR with a K_{D} of 4.2 nM to 5.2 nM, as measured by SPR.

80. The pharmaceutical formulation of any one of claims 1 to 79, wherein the multi-specific binding protein binds EGFR with an association rate constant of 1.5×10⁵ to 2.5×10⁵ l/Ms, as measured by SPR.

81. The pharmaceutical formulation of any one of claims 1 to 80, wherein the multi-specific binding protein binds EGFR with a dissociation rate constant of 9.0×10⁻⁴ to 10.0×10⁻⁴ l/s, as measured by SPR.

82. The pharmaceutical formulation of any one of claims 1 to 81, wherein the multi-specific binding protein binds NKG2D with a K_{D} of 4.50×10⁻⁴ mM to 5.20×10⁻⁴ mM, as measured by SPR.

83. The pharmaceutical formulation of any one of claims 1 to 82, wherein the multi-specific binding protein binds NKG2D with an association rate constant of 2.0×10⁵ to 2.6×10⁵ l/Ms, as measured by SPR.

84. The pharmaceutical formulation of any one of claims 1 to 83, wherein the multi-specific binding protein binds NKG2D with a dissociation rate constant of 0.6×10⁻¹ to 1.6×10⁻¹ l/s, as measured by SPR.

85. The pharmaceutical formulation of any one of claims 1 to 84, wherein the formulation is stable at room temperature for at least 1, at least 3, or at least 6 months.

86. The pharmaceutical formulation of any one of claims 1 to 85, wherein the formulation is stable at −80° C. for at least 1, at least 3, at least 6, at least 9, at least 12, at least 18, or at least 24 months.

87. The pharmaceutical formulation of claim 86, wherein the formulation is stable at −80° C. for at least 6 months.

88. The pharmaceutical formulation of claim 87, wherein the formulation is stable at −80° C. for at least 9 months.

89. The pharmaceutical formulation of claim 88, wherein the formulation is stable at −80° C. for at least 12 months.

90. The pharmaceutical formulation of any one of claims 1 to 89, wherein the formulation is stable at −20° C. for at least 1, at least 3, at least 6, at least 9, or at least 12 months.

91. The pharmaceutical formulation of claim 90, wherein the formulation is stable at −20° C. for at least 6 months.

92. The pharmaceutical formulation of any one of claims 1 to 91, wherein the formulation is stable at −5° C. for at least 1, at least 3, at least 6, at least 9, or at least 12 months.

93. The pharmaceutical formulation of claim 92, wherein the formulation is stable at −5° C. for at least 6 months.

94. The pharmaceutical formulation of any one of claims 1 to 93, wherein the formulation is stable at refrigerated temperatures for at least 1, at least 3, at least 6, at least 9, or at least 12 months.

95. The pharmaceutical formulation of any one of claims 1 to 94, wherein the multi-specific binding protein in the pharmaceutical formulation is capable of inhibiting EGFR signaling in EGFR-expressing cancer cells.

96. The pharmaceutical formulation of any one of claims 1 to 95, wherein the multi-specific binding protein in the pharmaceutical formulation is capable of activating NK cell-mediated killing of EGFR-expressing cancer cells.

97. The pharmaceutical formulation of any one of claims 1 to 96, wherein the multi-specific binding protein in the pharmaceutical formulation is capable of activating production and release of one or more chemokines and/or cytokines selected from IFNγ, TNFα, CCL4, CCL5, CXCL9, and CXCL10 from NK cells.

98. The pharmaceutical formulation of any one of claims 1 to 97, wherein the multi-specific binding protein in the pharmaceutical formulation is capable of activating CD8⁺ T cell killing of EGFR-expressing cancer cells.

99. The pharmaceutical formulation of any one of claims 1 to 98, wherein the multi-specific binding protein in the pharmaceutical formulation does not activate CD8⁺ T cells in the periphery.

100. The pharmaceutical formulation of any one of claims 1 to 99, wherein the multi-specific binding protein in the pharmaceutical formulation does not activate CD4⁺ T cells.

101. The pharmaceutical formulation of any one of claims 1 to 100, wherein the multi-specific binding protein in the pharmaceutical formulation is capable of binding human NKG2D and cynomolgus monkey NKG2D.

102. The pharmaceutical formulation of any one of claims 1 to 101, suitable for use in treating an unresectable solid tumor in a subject.

103. The pharmaceutical formulation of any one of claims 1 to 102, suitable for use in treating a recurrent solid tumor in a subject.

104. The pharmaceutical formulation of any one of claims 1 to 103, suitable for use in treating an advanced solid tumor in a subject for which there is no effective standard therapy.

105. The pharmaceutical formulation of any one of claims 1 to 104, suitable for use in treating a cancer in a subject that is intolerant of standard therapies.

106. The pharmaceutical formulation of any one of claims 102 to 105, wherein the pharmaceutical formulation is administered to the subject to achieve a multi-specific binding protein dose of 5 mg/kg to 50 mg/kg.

107. The pharmaceutical formulation of any one of claims 102 to 106, wherein the pharmaceutical formulation is administered to the subject once weekly in one or more 4-week treatment cycles.

108. The pharmaceutical formulation of claim 107, wherein the pharmaceutical formulation is administered to the subject on day 1, day 8, day 15, and day 22 of the one or more 4-week treatment cycles.

109. The pharmaceutical formulation of claim 107 or 108, wherein after a completed 4-week treatment cycle, the pharmaceutical formulation is administered to the subject to achieve an increased dose of the multi-specific binding protein in a subsequent 4-week treatment cycle as compared to the earlier completed 4-week treatment cycle.

110. The pharmaceutical formulation of any one of claims 1 to 109, wherein the pharmaceutical formulation is administered to the subject by intravenous infusion.

111. The pharmaceutical formulation of any one of claims 1 to 110, wherein the pharmaceutical formulation is suitable for use as a monotherapy.

112. The pharmaceutical formulation of any one of claims 102 to 110, wherein the pharmaceutical formulation is administered to the subject in combination with an anti-PD-1 or an anti-PD-L1 therapy.

113. The pharmaceutical formulation of claim 112, wherein the anti-PD-1 or anti-PD-L1 therapy is selected from nivolumab, pembrolizumab, durvalumab, or atezolizumab.

114. The pharmaceutical formulation of claim 113, wherein the anti-PD-1 or anti-PD-L1 therapy is nivolumab.

115. The pharmaceutical formulation of claim 114, wherein the nivolumab is administered at about 480 mg.

116. The pharmaceutical formulation of claim 114 or 115, wherein the nivolumab is administered on day 8 of each treatment cycle.

117. The pharmaceutical formulation of claim 113, wherein the anti-PD-1 or anti-PD-L1 therapy is pembrolizumab.

118. The pharmaceutical formulation of claim 117, wherein the pembrolizumab is administered at about 400 mg.

119. The pharmaceutical formulation of claim 117 or 118, wherein the pembrolizumab is administered once every 6 weeks.

120. The pharmaceutical formulation of any one of claims 112 to 119, wherein the subject is eligible for anti-PD-1 or an anti-PD-L1 therapy for a malignancy of epithelial origin.

121. The pharmaceutical formulation of claim 112, wherein no standard therapy exists or standard therapy of the subject has failed for a malignancy of epithelial origin.

122. The pharmaceutical formulation of claim 112 to 121, wherein the subject previously received anti-PD-1 or anti-PD-L1 therapy.

123. The pharmaceutical formulation of any one of claims 102 to 122, suitable for use in treating a head and neck squamous cell carcinoma (HNSCC) in the subject.

124. The pharmaceutical formulation of claim 123, wherein the HNSCC is a relapsed or metastatic HNSCC.

125. The pharmaceutical formulation of claim 123 or 124, wherein the subject has radiographic disease progression while on or after having received:
(i) pembrolizumab and platinum/5FU;
(ii) pembrolizumab monotherapy; or
(iii) platinum/5FU and cetuximab.

126. The pharmaceutical formulation of any one of claims 102 to 121, suitable for use in treating a colorectal cancer (CRC) in the subject.

127. The pharmaceutical formulation of claim 126, wherein the CRC is a relapsed or metastatic CRC.

128. The pharmaceutical formulation of claim 126 or 127, wherein the subject has been treated with FOLFOX, CAPOX, FOLFIRI, or FOLFOXIRI, with or without a biological agent.

129. The pharmaceutical formulation of any one of claims 126 to 128, wherein the subject does not have high mismatch repair/microsatellite instability.

130. The pharmaceutical formulation of any one of claims 126 to 129, wherein the subject has not had prior treatment with an anti-PD-1 or an anti-PD-L1 therapy.

131. The pharmaceutical formulation of any one of claims 126 to 130, wherein the subject has radiographic disease progression while or after receiving treatment for advanced (recurrent/unresectable/metastatic) cancer.

132. The pharmaceutical formulation of any one of claims 102 to 121, suitable for use in treating a non-small-cell lung cancer (NSCLC) in the subject.

133. The pharmaceutical formulation of claim 132, wherein the subject has recurrent or progressive disease during or after platinum doublet-based chemotherapy, or has recurrent or progressive disease within 6 months after completing platinum-based chemotherapy for local disease.

134. The pharmaceutical formulation of claim 132 or 133, wherein the subject has previously received an anti-PD-1 or anti-PD-L1 therapy.

135. The pharmaceutical formulation of any one of claims 102 to 122, suitable for use in treating an esophageal adenocarcinoma in the subject.

136. The pharmaceutical formulation of any one of claims 102 to 122, suitable for use in treating a triple-negative breast cancer in the subject.

137. The pharmaceutical formulation of any one of claims 102 to 122, suitable for use in treating a renal cell carcinoma in the subject.

138. The pharmaceutical formulation of any one of claims 102 to 122, suitable for use in treating a gastric cancer in the subject.

139. The pharmaceutical formulation of any one of claims 102 to 122, suitable for use in treating a pancreatic cancer in the subject.

140. The pharmaceutical formulation of any one of claims 102 to 139, wherein the pharmaceutical formulation is administered to the subject in combination with an effective amount of pre-medication comprising: (a) an antihistamine and an antipyretic or (b) a corticosteroid.

141. The pharmaceutical formulation of any one of claims 102 to 139, wherein the pharmaceutical formulation is administered to the subject in combination with an effective amount of pre-medication comprising: (a) an antihistamine and an antipyretic and (b) a corticosteroid.

142. The pharmaceutical formulation of claim 141, wherein the antihistamine and antipyretic are administered before each and every infusion of the pharmaceutical formulation, and the corticosteroid is administered before a first dose of a treatment cycle only.

143. The pharmaceutical formulation of any one of claims 140 to 142, wherein the antihistamine is diphenhydramine.

144. The pharmaceutical formulation of any one of claims 140 to 143, wherein the antipyretic is acetaminophen.

145. The pharmaceutical formulation of any one of claims 140 to 142, wherein the corticosteroid is methylprednisolone.

146. The pharmaceutical formulation of claim 145, wherein the methylprednisolone is administered to the subject at about 125 mg.

147. The pharmaceutical formulation of claim 145 or 146, wherein the methylprednisolone is administered to the subject within 60 minutes prior to the first dose of the pharmaceutical formulation.

148. The pharmaceutical formulation of any one of claims 102 to 147, wherein the pharmaceutical formulation inhibits EGFR signaling in the subject.

149. The pharmaceutical formulation of any one of claims 102 to 148, wherein the pharmaceutical formulation results in reduced anti-drug antibody (ADA) levels when administered to the subject relative to other anti-EGFR therapeutics.

150. The pharmaceutical formulation of claim 149, wherein the pharmaceutical formulation results in substantially no ADA production when administered to the subject.

151. The pharmaceutical formulation of any one of claims 102 to 150, wherein the pharmaceutical formulation results in reduced toxicity when administered to the subject relative to other anti-EGFR therapeutics.

152. The pharmaceutical formulation of claim 151, wherein toxicity comprises one or more of skin toxicity, keratitis, ulcerative keratitis, corneal perforation, diarrhea, hypomagnesemia, infusion-related reactions, thrombocytopenia, neutropenia, fatigue, hypertension, vomiting, and nausea.

153. The pharmaceutical formulation of any one of claims 1 to 152, wherein the subject is diagnosed as having an EGFR-positive cancer, as determined by immunohistochemistry.

154. The pharmaceutical formulation of any one of claims 1 to 153, wherein the subject is diagnosed as having an EGFR-positive cancer, wherein the cancer has an activating mutation or gene amplification of the EGFR gene.

155. The pharmaceutical formulation of claim 154, wherein EGFR gene amplification is determined by fluorescent in situ hybridization.

156. The pharmaceutical formulation of claim 154, wherein EGFR activating mutation or gene amplification is determined by DNA sequencing.

157. A kit comprising the pharmaceutical formulation of any one of claims 1 to 156 and instructions for use.

158. A method of treating cancer in a subject in need thereof, the method comprising administering an effective amount of a multi-specific binding protein comprising:
(a) a first antigen-binding site that binds NKG2D;
(b) a second antigen-binding site that binds EGFR; and
(c) an antibody Fc domain or a portion thereof sufficient to bind CD16, or a third antigen-binding site that binds CD16,
wherein the cancer is an unresectable solid tumor.

159. A method of treating cancer in a subject in need thereof, the method comprising administering an effective amount of a multi-specific binding protein comprising:
(a) a first antigen-binding site that binds NKG2D;
(b) a second antigen-binding site that binds EGFR; and
(c) an antibody Fc domain or a portion thereof sufficient to bind CD16, or a third antigen-binding site that binds CD16,
wherein the cancer is a recurrent solid tumor.

160. A method of treating cancer in a subject in need thereof, the method comprising administering an effective amount of a multi-specific binding protein comprising:
(a) a first antigen-binding site that binds NKG2D;
(b) a second antigen-binding site that binds EGFR; and
(c) an antibody Fc domain or a portion thereof sufficient to bind CD16, or a third antigen-binding site that binds CD16,
wherein the cancer is an advanced solid tumor for which there is no effective standard therapy.

161. A method of treating cancer in a subject in need thereof, the method comprising administering an effective amount of a multi-specific binding protein comprising:
(a) a first antigen-binding site that binds NKG2D;
(b) a second antigen-binding site that binds EGFR; and
(c) an antibody Fc domain or a portion thereof sufficient to bind CD16, or a third antigen-binding site that binds CD16,
wherein the subject is intolerant of standard therapies.

162. A method of treating cancer in a subject in need thereof, the method comprising administering an effective amount of a multi-specific binding protein in combination with nivolumab, wherein the multi-specific binding protein comprises:
(a) a first antigen-binding site that binds NKG2D;
(b) a second antigen-binding site that binds EGFR; and
(c) an antibody Fc domain or a portion thereof sufficient to bind CD16, or a third antigen-binding site that binds CD16.

163. A method of treating cancer in a subject in need thereof, the method comprising administering 5 mg/kg to 50 mg/kg of a multi-specific binding protein comprising:
(a) a first antigen-binding site that binds NKG2D;
(b) a second antigen-binding site that binds EGFR; and
(c) an antibody Fe domain or a portion thereof sufficient to bind CD16, or a third antigen-binding site that binds CD16.

164. A method of treating cancer in a subject in need thereof, the method comprising administering a multi-specific binding protein once weekly in 4-week treatment cycles, wherein the multi-specific binding protein comprises:
(a) a first antigen-binding site that binds NKG2D;
(b) a second antigen-binding site that binds EGFR; and
(c) an antibody Fc domain or a portion thereof sufficient to bind CD16, or a third antigen-binding site that binds CD16.

165. A method of treating cancer in a subject in need thereof, the method comprising administering an effective amount of a multi-specific binding protein in combination with an anti-PD-1 or an anti-PD-L1 therapy, wherein the multi-specific binding protein comprises:
(a) a first antigen-binding site that binds NKG2D;
(b) a second antigen-binding site that binds EGFR; and
(c) an antibody Fc domain or a portion thereof sufficient to bind CD16, or a third antigen-binding site that binds CD16, and
wherein the subject is eligible for anti-PD-1 or an anti-PD-L1 therapy for a malignancy of epithelial origin.

166. A method of treating cancer in a subject in need thereof, the method comprising administering an effective amount of a multi-specific binding protein in combination with an anti-PD-1 or an anti-PD-L1 therapy, wherein the multi-specific binding protein comprises:
(a) a first antigen-binding site that binds NKG2D;
(b) a second antigen-binding site that binds EGFR; and
(c) an antibody Fe domain or a portion thereof sufficient to bind CD16, or a third antigen-binding site that binds CD16, and
wherein no standard therapy exists or standard therapy of the subject has failed for a malignancy of epithelial origin.

167. A method of treating cancer in a subject in need thereof, the method comprising administering an effective amount of a multi-specific binding protein in combination with an anti-PD-1 or an anti-PD-L1 therapy, wherein the multi-specific binding protein comprises:
(a) a first antigen-binding site that binds NKG2D;
(b) a second antigen-binding site that binds EGFR; and
(c) an antibody Fc domain or a portion thereof sufficient to bind CD16, or a third antigen-binding site that binds CD16, and
wherein the subject has previously received an anti-PD-1 or anti-PD-L1 therapy.

168. A method of treating head and neck squamous cell carcinoma (HNSCC) in a subject in need thereof, the method comprising administering an effective amount of a multi-specific binding protein comprising:
(a) a first antigen-binding site that binds NKG2D;
(b) a second antigen-binding site that binds EGFR; and
(c) an antibody Fc domain or a portion thereof sufficient to bind CD16, or a third antigen-binding site that binds CD16.

169. The method of claim 168, wherein the HNSCC is a relapsed or metastatic HNSCC.

170. The method of claim 168 or 169, wherein the subject has radiographic disease progression while on or after having received:
(i) pembrolizumab and platinum/5FU;
(ii) pembrolizumab monotherapy; or
(iii) platinum/5FU and cetuximab.

171. A method of treating colorectal cancer (CRC) in a subject in need thereof, the method comprising administering an effective amount of a multi-specific binding protein comprising:
(a) a first antigen-binding site that binds NKG2D;
(b) a second antigen-binding site that binds EGFR; and
(c) an antibody Fc domain or a portion thereof sufficient to bind CD16, or a third antigen-binding site that binds CD16,
wherein the CRC is a relapsed or metastatic CRC.

172. A method of treating colorectal cancer (CRC) in a subject in need thereof, the method comprising administering an effective amount of a multi-specific binding protein comprising:
(a) a first antigen-binding site that binds NKG2D;
(b) a second antigen-binding site that binds EGFR; and
(c) an antibody Fc domain or a portion thereof sufficient to bind CD16, or a third antigen-binding site that binds CD16,
wherein the subject has been treated with FOLFOX, CAPOX, FOLFIRI, or FOLFOXIRI, with or without a biological agent.

173. A method of treating colorectal cancer (CRC) in a subject in need thereof, the method comprising administering an effective amount of a multi-specific binding protein comprising:
(a) a first antigen-binding site that binds NKG2D;
(b) a second antigen-binding site that binds EGFR; and
(c) an antibody Fe domain or a portion thereof sufficient to bind CD16, or a third antigen-binding site that binds CD16,
wherein the subject does not have high mismatch repair/microsatellite instability.

174. A method of treating colorectal cancer (CRC) in a subject in need thereof, the method comprising administering an effective amount of a multi-specific binding protein comprising:
(a) a first antigen-binding site that binds NKG2D;
(b) a second antigen-binding site that binds EGFR; and
(c) an antibody Fc domain or a portion thereof sufficient to bind CD16, or a third antigen-binding site that binds CD16,
wherein the subject has not had prior treatment with an anti-PD-1 or an anti-PD-L1 therapy.

175. A method of treating colorectal cancer (CRC) in a subject in need thereof, the method comprising administering an effective amount of a multi-specific binding protein comprising:
(a) a first antigen-binding site that binds NKG2D;
(b) a second antigen-binding site that binds EGFR; and
(c) an antibody Fc domain or a portion thereof sufficient to bind CD16, or a third antigen-binding site that binds CD16,
wherein the subject has radiographic disease progression while or after receiving treatment for advanced (recurrent/unresectable/metastatic) cancer.

176. A method of treating non-small-cell lung cancer (NSCLC) in a subject in need thereof, the method comprising administering an effective amount of a multi-specific binding protein comprising:
(a) a first antigen-binding site that binds NKG2D;
(b) a second antigen-binding site that binds EGFR; and
(c) an antibody Fe domain or a portion thereof sufficient to bind CD16, or a third antigen-binding site that binds CD16,
wherein the subject has recurrent or progressive disease during or after platinum doublet-based chemotherapy, or has recurrent or progressive disease within 6 months after completing platinum-based chemotherapy for local disease.

177. A method of treating non-small-cell lung cancer (NSCLC) in a subject in need thereof, the method comprising administering an effective amount of a multi-specific binding protein comprising:
(a) a first antigen-binding site that binds NKG2D;
(b) a second antigen-binding site that binds EGFR; and
(c) an antibody Fc domain or a portion thereof sufficient to bind CD16, or a third antigen-binding site that binds CD16,
wherein the subject has previously received an anti-PD-1 or anti-PD-L1 therapy.

178. A method of treating esophageal adenocarcinoma in a subject in need thereof, the method comprising administering an effective amount of a multi-specific binding protein comprising:
(a) a first antigen-binding site that binds NKG2D;
(b) a second antigen-binding site that binds EGFR; and
(c) an antibody Fc domain or a portion thereof sufficient to bind CD16, or a third antigen-binding site that binds CD16.

179. A method of treating triple-negative breast cancer in a subject in need thereof, the method comprising administering an effective amount of a multi-specific binding protein comprising:
(a) a first antigen-binding site that binds NKG2D;
(b) a second antigen-binding site that binds EGFR; and
(c) an antibody Fe domain or a portion thereof sufficient to bind CD16, or a third antigen-binding site that binds CD16.

180. A method of treating renal cell carcinoma in a subject in need thereof, the method comprising administering an effective amount of a multi-specific binding protein comprising:
(a) a first antigen-binding site that binds NKG2D;
(b) a second antigen-binding site that binds EGFR, comprising a VH having CDR1, CDR2, and CDR3 sequences selected from a group consisting of:
(i) SEQ ID NOs: 136, 157, and 138, respectively,
(ii) SEQ ID NOs: 136, 146, and 138, respectively, and
(iii) SEQ ID NOs: 136, 137, and 138, respectively; and
a light chain variable domain (VL) having CDR1, CDR2, and CDR3 sequences selected from a group consisting of,
(i) SEQ ID NOs: 140, 141, and 151, respectively, and
(ii) SEQ ID NOs: 140, 141, and 142, respectively; and
(c) an antibody Fc domain or a portion thereof sufficient to bind CD16, or a third antigen-binding site that binds CD16.

181. A method of inhibiting EGFR signaling in a subject in need thereof, the method comprising administering to the subject a multi-specific binding protein comprising:
(i) a first antigen-binding site that binds NKG2D;
(ii) a second antigen-binding site that binds EGFR; and
(iii) an antibody Fc domain or a portion thereof sufficient to bind CD16, or a third antigen-binding site that binds CD16.

182. The method of any one of claims 158 to 181, wherein the multi-specific binding protein is administered as a pharmaceutical formulation comprising one or more of:
(a) citrate;
(b) a sugar or sugar alcohol; and
(c) a polysorbate
at pH 6.0 to 7.0.

183. A method of treating renal cell carcinoma in a subject in need thereof, the method comprising administering an effective amount of a multi-specific binding protein comprising:
(a) a first antigen-binding site that binds NKG2D;
(b) a second antigen-binding site that binds EGFR, comprising a VH having CDR1, CDR2, and CDR3 sequences selected from a group consisting of:
(i) SEQ ID NOs: 136, 157, and 138, respectively,
(ii) SEQ ID NOs: 136, 146, and 138, respectively, and
(iii) SEQ ID NOs: 136, 137, and 138, respectively; and
a VL having CDR1, CDR2, and CDR3 sequences selected from a group consisting of,
(i) SEQ ID NOs: 140, 141, and 151, respectively, and
(ii) SEQ ID NOs: 140, 141, and 142, respectively; and
(c) an antibody Fc domain or a portion thereof sufficient to bind CD16, or a third antigen-binding site that binds CD16,
wherein the multi-specific binding protein is administered as a pharmaceutical formulation comprising or more of:
(a) citrate;
(b) a sugar or sugar alcohol; and
(c) a polysorbate
at pH 6.0 to 7.0.

184. A method of treating gastric cancer in a subject in need thereof, the method comprising administering an effective amount of a multi-specific binding protein comprising:
(a) a first antigen-binding site that binds NKG2D;
(b) a second antigen-binding site that binds EGFR, comprising a VH having CDR1, CDR2, and CDR3 sequences selected from a group consisting of:
(i) SEQ ID NOs: 136, 157, and 138, respectively,
(ii) SEQ ID NOs: 136, 146, and 138, respectively, and
(iii) SEQ ID NOs: 136, 137, and 138, respectively; and
a VL having CDR1, CDR2, and CDR3 sequences selected from a group consisting of,
(i) SEQ ID NOs: 140, 141, and 151, respectively, and
(ii) SEQ ID NOs: 140, 141, and 142, respectively; and
(c) an antibody Fc domain or a portion thereof sufficient to bind CD16, or a third antigen-binding site that binds CD16,
wherein the multi-specific binding protein is administered as a pharmaceutical formulation comprising or more of:
(a) citrate;
(b) a sugar or sugar alcohol; and
(c) a polysorbate
at pH 6.0 to 7.0.

185. A method of treating pancreatic cancer in a subject in need thereof, the method comprising administering an effective amount of a multi-specific binding protein comprising:
(a) a first antigen-binding site that binds NKG2D;
(b) a second antigen-binding site that binds EGFR, comprising a VH having CDR1, CDR2, and CDR3 sequences selected from a group consisting of:
(i) SEQ ID NOs: 136, 157, and 138, respectively,
(ii) SEQ ID NOs: 136, 146, and 138, respectively, and
(iii) SEQ ID NOs: 136, 137, and 138, respectively; and
a VL having CDR1, CDR2, and CDR3 sequences selected from a group consisting of,
(i) SEQ ID NOs: 140, 141, and 151, respectively, and
(ii) SEQ ID NOs: 140, 141, and 142, respectively; and
(c) an antibody Fc domain or a portion thereof sufficient to bind CD16, or a third antigen-binding site that binds CD16,
wherein the multi-specific binding protein is administered as a pharmaceutical formulation comprising or more of:
(a) citrate;
(b) a sugar or sugar alcohol; and
(c) a polysorbate
at pH 6.0 to 7.0.

186. The method of any one of claims 158 to 185, wherein the multi-specific binding protein is administered as a pharmaceutical formulation comprising one or more of:
(a) 15 mM to 25 mM citrate; and
(b) 4% to 8% (w/v) mannitol,
at pH 6.0 to 7.0.

187. The method of claim 186, wherein the pharmaceutical formulation further comprises a polysorbate.

188. The method of any one of claims 182 to 187, wherein the concentration of the multi-specific binding protein in the pharmaceutical formulation is 1 mg/mL to 125 mg/mL.

189. The method of any one of claims 182 or 188, wherein the concentration of the multi-specific binding protein in the pharmaceutical formulation is 2 mg/mL to 100 mg/mL.

190. The method of any one of claims 182 to 189, wherein the concentration of the multi-specific binding protein in the pharmaceutical formulation is 5 mg/mL to 50 mg/mL.

191. The method of any one of claims 182 to 190, wherein the concentration of the multi-specific binding protein in the pharmaceutical formulation is 5 mg/mL to 20 mg/mL.

192. The method of any one of claims 182 to 191, wherein the concentration of the multi-specific binding protein in the pharmaceutical formulation is 10 mg/mL to 20 mg/mL.

193. The method of any one of claims 182 to 192, wherein the concentration of the multi-specific binding protein in the pharmaceutical formulation is about 15 mg/mL.

194. The method of any one of claims 182 to 193, wherein the pharmaceutical formulation is diluted with a suitable diluent in the range of 1:0 to 1:10 prior to administration to a subject.

195. The method of any one of claims 182 to 194, wherein the pharmaceutical formulation comprises 15 mM to 25 mM citrate.

196. The method of any one of claims 182 to 195, wherein the pharmaceutical formulation comprises 17.5 mM to 22.5 mM citrate.

197. The method of any one of claims 182 to 196, wherein the pharmaceutical formulation comprises about 20 mM citrate.

198. The method of any one of claims 182 to 185, or 188 to 197, wherein the sugar alcohol is an alcohol of a monosaccharide.

199. The method of any one of claims 182 to 185, or 188 to 198, wherein the sugar alcohol is mannitol.

200. The method of any one of claims 182 to 199, wherein the pharmaceutical composition comprises 4% to 8% (w/v) mannitol.

201. The method of any one of claims 182 to 200, wherein the pharmaceutical formulation comprises 5% to 7% (w/v) mannitol.

202. The method of claim 201, wherein the pharmaceutical formulation comprises 6% (w/v) mannitol.

203. The method of any one of claims 182 to 185, or 187 to 202, wherein the polysorbate is polysorbate 80.

204. The method of any one of claims 182 to 185, or 187 to 203, wherein the pharmaceutical formulation comprises 0.005% to 0.05% (w/v) polysorbate 80.

205. The method of any one of claims 182 to 185, or 187 to 204, wherein the pharmaceutical formulation comprises 0.0075% to 0.025% (w/v) polysorbate 80.

206. The method of any one of claims 182 to 185, or 187 to 205, wherein the pharmaceutical formulation comprises about 0.01% (w/v) polysorbate 80.

207. The method of any one of claims 182 to 206, wherein the pH is 6.2 to 6.8.

208. The method of any one of claims 182 to 207, wherein the pH is 6.4 to 6.6.

209. The method of any one of claims 182 to 208, wherein the pH is about 6.5.

210. The method of any one of claims 182 to 190, 194, 195, 198 to 200, 203, 204, or 207, wherein the formulation comprises:
(a) 5 mg/mL to 50 mg/mL of the multi-specific binding protein;
(b) 15 mM to 25 mM citrate;
(c) 4% to 8% (w/v) mannitol; and
(d) 0.005% to 0.05% (w/v) polysorbate 80,
at pH 6.2 to 6.8.

211. The method of any one of claims 182 to 192, 194 to 196, 198 to 201, 203 to 205, 207, or 208, wherein the formulation comprises:
(a) 10 mg/mL to 20 mg/mL of the multi-specific binding protein;
(b) 17.5 mM to 22.5 mM citrate;
(c) 5% to 7% (w/v) mannitol; and
(d) 0.0075% to 0.025% (w/v) polysorbate 80,
at pH 6.4 to 6.6.

212. The method of any one of claims 182 to 211, wherein the formulation comprises:
(a) about 15 mg/mL of the multi-specific binding protein;
(b) about 20 mM citrate;
(c) about 6% (w/v) mannitol; and
(d) about 0.01% (w/v) polysorbate 80,
at about pH 6.5.

213. The method of any one of claims 158 to 212, wherein:
(a) the first antigen-binding site of the multi-specific binding protein comprises a heavy chain variable domain (VH) having complementarity-determining region 1 (CDR1), complementarity-determining region 2 (CDR2), and complementarity-determining region 3 (CDR3) sequences selected from a group consisting of,
(i) SEQ ID NOs: 81, 82, and 112, respectively, and
(ii) SEQ ID NOs: 81, 82, and 97, respectively, and
a light chain variable domain (VL) having CDR1, CDR2, and CDR3 sequences of SEQ ID NOs: 86, 77, and 87, respectively;
(b) the second antigen-binding site of the multi-specific binding protein comprises a VH having CDR1, CDR2, and CDR3 sequences selected from a group consisting of,
(i) SEQ ID NOs: 136, 157, and 138, respectively,
(ii) SEQ ID NOs: 136, 146, and 138, respectively, and
(iii) SEQ ID NOs: 136, 137, and 138, respectively, and
a light chain variable domain (VL) having CDR1, CDR2, and CDR3 sequences selected from a group consisting of,
(iv) SEQ ID NOs: 140, 141, and 151, respectively, and
(v) SEQ ID NOs: 140, 141, and 142, respectively.

214. The method of claim 213, wherein:
(a) the VH of the first antigen-binding site of the multi-specific binding protein comprises an amino acid sequence at least 90% identical to an amino acid sequence selected from a group consisting of SEQ ID NO:110 and SEQ ID NO:95, and the VL of the first antigen-binding site of the multi-specific binding protein comprises an amino acid sequence at least 90% identical to SEQ ID NO:85; and
(b) the VH of the second antigen-binding site of the multi-specific binding protein comprises an amino acid sequence at least 90% identical to an amino acid sequence selected from a group consisting of SEQ ID NO:156, SEQ ID NO:145, SEQ ID NO:170, and SEQ ID NO:135, and the VL of the second antigen-binding site of the multi-specific binding protein comprises an amino acid sequence at least 90% identical to an amino acid sequence selected from a group consisting of SEQ ID NO:150, SEQ ID NO:171, and SEQ ID NO:147.

215. The method of claim 213 or 214, wherein the second antigen-binding site comprises a single-chain variable fragment (scFv) comprising an amino acid sequence at least 90% identical to an amino acid sequence selected from a group consisting of SEQ ID NO:158, SEQ ID NO:159, SEQ ID NO:148, SEQ ID NO:149, SEQ ID NO:152, SEQ ID NO:153, SEQ ID NO:154, and SEQ ID NO:155.

216. The method of any one of claims 158 to 212, wherein the first antigen-binding site of the multi-specific binding protein comprises a Fab and the second antigen-binding site of the multi-specific binding protein comprises a single-chain variable fragment (scFv), and wherein the scFv comprises a heavy chain variable domain (VH) and a light chain variable domain (VL).

217. The method of any one of claims 213 to 216, wherein the VL of the scFv is linked to the VH of the scFv via a flexible linker.

218. The method of claim 217, wherein the flexible linker comprises the amino acid sequence of SEQ ID NO:119.

219. The method of claim 217 or 218, wherein the flexible linker consists of the amino acid sequence of SEQ ID NO:119.

220. The method of any one of claims 216 to 219, wherein the VL of the scFv is positioned to the N-terminus of the VH of the scFv, or the VH of the scFv is positioned to the N-terminus of the VL of the scFv.

221. The method of any one of claims 216 to 220, wherein the VH of the scFv forms a disulfide bridge with the VL of the scFv.

222. The method of claim 221, wherein the disulfide bridge is formed between C44 of the VH of the scFv and C100 of the VL of the scFv, numbered under the Kabat numbering scheme.

223. The method of any one of claims 158 to 222, wherein the antibody Fc domain comprises a first antibody Fc polypeptide linked to the Fab and a second antibody Fc polypeptide linked to the scFv.

224. The method of claim 223, wherein the first antibody Fc polypeptide is linked to a heavy chain portion of the Fab.

225. The method of claim 223 or 224, wherein the scFv is linked to the second antibody Fc polypeptide via a hinge comprising Ala-Ser or Gly-Ser.

226. The method of any one of claims 223 to 225, wherein the first and second antibody Fc polypeptides each comprise a hinge and a CH2 domain of a human IgG1 antibody.

227. The method of claim 226, wherein the first and second antibody Fc polypeptides each comprise an amino acid sequence at least 90% identical to amino acids 234-332 of a wild-type human IgG1 antibody, numbered according to the EU index.

228. The method of any one of claims 223 to 227, wherein the first and second antibody Fc polypeptides each comprise different mutations promoting heterodimerization.

229. The method of claim 228, wherein the first antibody Fc polypeptide is a human IgG1 Fc polypeptide comprising K360E and K409W substitutions, numbered according to the EU index.

230. The method of claim 228 or 229, wherein the second antibody Fc polypeptide is a human IgG1 Fc polypeptide comprising Q347R, D399V, and F405T substitutions, numbered according to the EU index.

231. The method of any one of claims 158 to 212, wherein the first-antigen binding site comprises a Fab comprising:
(a) a VH comprising CDR1, CDR2, and CDR3 sequences of SEQ ID NOs: 81, 82, and 112, respectively; and
(b) a VL comprising CDR1, CDR2, and CDR3 sequences of SEQ ID NOs: 86, 77, and 87, respectively.

232. The method of claim 231, wherein:
(a) the VH of the Fab comprises CDR1, CDR2, and CDR3 sequences of SEQ ID NOs: 81, 82, and 97, respectively; and
(b) the VL of the Fab comprises CDR1, CDR2, and CDR3 sequences of SEQ ID NOs: 86, 77, and 87, respectively.

233. The method of claim 231 or 232, wherein the VH of the Fab comprises an amino acid sequence at least 90% identical to SEQ ID NO:95 or an amino acid sequence at least 90% identical to SEQ ID NO:110, and the VL of the Fab comprises an amino acid sequence at least 90% identical to SEQ ID NO:85.

234. The method of any one of claims 231 to 233, wherein the VH of the Fab comprises the amino acid sequence of SEQ ID NO:95, and the VL of the Fab comprises the amino acid sequence of SEQ ID NO:85.

235. The method of any one of claims 158 to 179, 181, 182, 186 to 212, or 231 to 234, wherein the second antigen-binding site comprises a single-chain variable fragment (scFv) comprising:
a) a VH comprising CDR1, CDR2, and CDR3 sequences of SEQ ID NOs: 136, 157, and 138, respectively; and a VL comprising CDR1, CDR2, and CDR3 sequences of SEQ ID NOs: 140, 141, and 151, respectively; or
b) a VH comprising CDR1, CDR2, and CDR3 sequences of SEQ ID NOs: 136, 146, and 138, respectively; and a VL comprising CDR1, CDR2, and CDR3 sequences of SEQ ID NOs: 140, 141, and 142, respectively.

236. The method of claim 235, wherein the VH of the scFv comprises CDR1, CDR2, and CDR3 sequences of SEQ ID NOs: 136, 157, and 138, respectively; and the VL of the scFv comprises CDR1, CDR2, and CDR3 sequences of SEQ ID NOs: 140, 141, and 151, respectively.

237. The method of claims 235 or 236, wherein the VH of the scFv comprises an amino acid sequence at least 90% identical to SEQ ID NO:156, and the VL of the scFv comprises an amino acid sequence at least 90% identical to SEQ ID NO:150.

238. The method of any one of claims 235 to 237, wherein the scFv comprises the amino acid sequence of SEQ ID NO:158 or SEQ ID NO:159.

239. The method of any one of claims 158 to 179, 181, 182, 186 to 212, or 231 to 234, wherein the second antigen binding site comprises an scFv comprising a VH comprising CDR1, CDR2, and CDR3 sequences of SEQ ID NOs: 136, 146, and 138, respectively; and a VL comprising CDR1, CDR2, and CDR3 sequences of SEQ ID NOs: 140, 141, and 151, respectively.

240. The method of claim 239, wherein the VH of the scFv comprises an amino acid sequence at least 90% identical to SEQ ID NO:170, or at least 90% identical to SEQ ID NO:145, and the VL of the scFv comprises an amino acid sequence at least 90% identical to SEQ ID NO:171, or at least 90% identical to SEQ ID NO:150.

241. The method of claim 239 or 240, wherein the VH of the scFv comprises the amino acid sequence of SEQ ID NO:170, and the VL of the scFv comprises the amino acid sequence of SEQ ID NO:171.

242. The method of any one of claims 239 to 241, wherein the scFv comprises the amino acid sequence of SEQ ID NO:152 or SEQ ID NO:153.

243. The method of any one of claims 158 to 222, wherein:
(a) the first antigen-binding site comprises a Fab comprising a VH comprising CDR1, CDR2, and CDR3 sequences of SEQ ID NOs: 81, 82, and 97, respectively; and a VL comprising CDR1, CDR2, and CDR3 sequences of SEQ ID NOs: 86, 77, and 87, respectively;
(b) the second-antigen-binding site comprises a single-chain variable fragment (scFv) comprising a VH comprising CDR1, CDR2, and CDR3 sequences of SEQ ID NOs: 136, 146, and 138, respectively; and a VL comprising CDR1, CDR2, and CDR3 sequences of SEQ ID NOs: 140, 141, and 151, respectively; and
(c) the antibody Fc domain comprises a first antibody Fc polypeptide linked to the Fab and a second antibody Fc polypeptide linked to the scFv, wherein the first antibody Fc polypeptide is a human IgG1 Fc polypeptide comprising K360E and K409W substitutions, and the second antibody Fc polypeptide is a human IgG1 Fc polypeptide comprising Q347R, D399V, and F405T substitutions, numbered according to the EU index.

244. The method of claim 243, wherein:
(a) the VH of the Fab comprises an amino acid sequence at least 90% identical to SEQ ID NO:95, or an amino acid sequence at least 90% identical to SEQ ID NO:110, and the VL of the Fab comprises an amino acid sequence at least 90% identical to SEQ ID NO:85; and
(b) the VH of the scFv comprises an amino acid sequence at least 90% identical to an amino acid sequence selected from the group consisting of SEQ ID NO:156, SEQ ID NO:145, SEQ ID NO:170, and SEQ ID NO:135, and the VL of the scFv comprises an amino acid sequence at least 90% identical to an amino acid sequence selected from the group consisting of SEQ ID NO:150, SEQ ID NO:147, and SEQ ID NO:171.

245. The method of claim 243, or 244, wherein:
(a) the VH of the Fab comprises the amino acid sequence of SEQ ID NO:95, and the VL of the Fab comprises the amino acid sequence of SEQ ID NO:85; and
(b) the scFv comprises the amino acid sequence of SEQ ID NO:152 or SEQ ID NO:153.

246. The method of any one of claims 158 to 245, wherein the multi-specific binding protein comprises:
(a) a first polypeptide comprising the amino acid sequence of SEQ ID NO:167;
(b) a second polypeptide comprising the amino acid sequence of SEQ ID NO:164; and
(c) a third polypeptide comprising the amino acid sequence of SEQ ID NO:165.

247. The method of any one of claims 239 to 246, wherein the multi-specific binding protein is administered as a pharmaceutical formulation comprising:
(a) about 15 mg/mL of the multi-specific binding protein;
(b) about 20 mM citrate;
(c) about 6% (w/v) mannitol; and
(d) about 0.01% (w/v) polysorbate 80,
at about pH 6.5.

248. The method of any one of claims 158 to 179, 181, 182, 186 to 212 or 231 to 234, wherein the second antigen binding site comprises an scFv comprising a VH comprising CDR1, CDR2, and CDR3 sequences of SEQ ID NOs: 136, 137, and 138, respectively; and a VL comprising CDR1, CDR2, and CDR3 sequences of SEQ ID NOs: 140, 141, and 151, respectively.

249. The method of claim 248, wherein the VH of the scFv comprises an amino acid sequence at least 90% identical to SEQ ID NO:135, and the VL of the scFv comprises an amino acid sequence at least 90% identical to SEQ ID NO:150.

250. The method of claim 248 or 249, wherein the VH of the scFv comprises the amino acid sequence of SEQ ID NO:135, wherein the glycine at position 44 of the VH is substituted with a cysteine (C44), numbered under the Kabat numbering scheme, and the VL of the scFv comprises the amino acid sequence of SEQ ID NO:150, wherein the glycine at position 100 of the VL is substituted with a cysteine (C100), numbered under the Kabat numbering scheme, and wherein the C44 and the C100 form a disulfide bond between the VH and VL.

251. The method of any one of claims 248 to 250, wherein the scFv comprises the amino acid sequence of SEQ ID NO:154 or SEQ ID NO:155.

252. The method of any one of claims 158 to 179, 181, 182, 186 to 222, wherein:
(a) the first antigen-binding site comprises a Fab comprising a VH comprising CDR1, CDR2, and CDR3 sequences of SEQ ID NOs: 81, 82, and 97, respectively; and a VL comprising CDR1, CDR2, and CDR3 sequences of SEQ ID NOs: 86, 77, and 87, respectively;
(b) the second antigen-binding site comprises a single-chain variable fragment (scFv) comprising a VH comprising CDR1, CDR2, and CDR3 sequences of SEQ ID NOs: 136, 137, and 138, respectively; and a VL comprising CDR1, CDR2, and CDR3 sequences of SEQ ID NOs: 140, 141, and 151, respectively; and
(c) the antibody Fc domain comprises a first antibody Fc polypeptide linked to the Fab and a second antibody Fc polypeptide linked to the scFv, wherein the first antibody Fc polypeptide is a human IgG1 Fc polypeptide comprising K360E and K409W substitutions, and the second antibody Fc polypeptide is a human IgG1 Fc polypeptide comprising Q347R, D399V, and F405T substitutions, numbered according to the EU index.

253. The method of claim 252, wherein:
(a) the VH of the Fab comprises an amino acid sequence at least 90% identical to SEQ ID NO:95 or at least 90% identical to SEQ ID NO:110, and the VL of the scFv comprises an amino acid sequence at least 90% identical to SEQ ID NO:85; and
(b) the VH of the scFv comprises an amino acid sequence at least 90% identical to the amino acid sequence of SEQ ID NO:135, and the VL of the scFv comprises an amino acid sequence at least 90% identical to the amino acid sequence of SEQ ID NO:150.

254. The method of claim 252 or 253, wherein:
(a) the VH of the Fab comprises the amino acid sequence of SEQ ID NO:95, and the VL of the Fab comprises the amino acid sequence of SEQ ID NO:85; and
(b) the scFv comprises the amino acid sequence of SEQ ID NO:154 or SEQ ID NO:155.

255. The method of any one of claims 248 to 254, wherein the multi-specific binding protein is administered as a pharmaceutical formulation comprising:
(a) about 15 mg/mL of the multi-specific binding protein;
(b) about 20 mM citrate;
(c) about 6% (w/v) mannitol; and
(d) about 0.01% (w/v) polysorbate 80,
at about pH 6.5.

256. The method of any one of claims 158 to 212, or 231 to 234, wherein the second antigen-binding site comprises a single-chain variable fragment (scFv) comprising a VH comprising CDR1, CDR2, and CDR3 sequences of SEQ ID NOs: 136, 146, and 138, respectively; and a VL comprising CDR1, CDR2, and CDR3 sequences of SEQ ID NOs: 140, 141, and 142, respectively.

257. The method of claim 256, wherein the VH of the scFv comprises an amino acid sequence at least 90% identical to SEQ ID NO:145, and the VL of the scFv comprises an amino acid sequence at least 90% identical to SEQ ID NO:147.

258. The method of claim 256, or 257, wherein the VH of the scFv comprises the amino acid sequence of SEQ ID NO:145, wherein the glycine at position 44 of the VH is substituted with a cysteine (C44), numbered under the Kabat numbering scheme, and the VL of the scFv comprises the amino acid sequence of SEQ ID NO:147, wherein the glycine at position 100 of the VL is substituted with a cysteine (C100), numbered under the Kabat numbering scheme, and wherein the C44 and the C100 form a disulfide bond between the VH and VL.

259. The method of any one of claims 256 to 258, wherein the scFv comprises the amino acid sequence of SEQ ID NO:148 or SEQ ID NO:149.

260. The method of any one of claims 158 to 222, wherein:
(a) the first antigen-binding site comprises a Fab comprising a VH comprising CDR1, CDR2, and CDR3 sequences of SEQ ID NOs: 81, 82, and 97, respectively; and a VL comprising CDR1, CDR2, and CDR3 sequences of SEQ ID NOs: 86, 77, and 87, respectively;
(b) the second antigen-binding site comprises a single-chain variable fragment (scFv) comprising a VH comprising CDR1, CDR2, and CDR3 sequences of SEQ ID NOs: 136, 146, and 138, respectively; and a VL comprising CDR1, CDR2, and CDR3 sequences of SEQ ID NOs: 140, 141, and 142, respectively; and
(c) the antibody Fc domain comprises a first antibody Fc polypeptide linked to the Fab and a second antibody Fc polypeptide linked to the scFv, wherein the first antibody Fc polypeptide is a human IgG1 Fc polypeptide comprising K360E and K409W substitutions, and the second antibody Fc polypeptide is a human IgG1 Fc polypeptide comprising Q347R, D399V, and F405T substitutions, numbered according to the EU index.

261. The method of claim 260, wherein:
(a) the VH of the Fab comprises an amino acid sequence at least 90% identical to SEQ ID NO:95 or at least 90% identical to SEQ ID NO:110, and the VL of the Fab comprises an amino acid sequence at least 90% identical to SEQ ID NO:85; and
(b) the VH of the scFv comprises an amino acid sequence at least 90% identical to the amino acid sequence of SEQ ID NO:145, and the VL of the scFv comprises an amino acid sequence at least 90% identical to the amino acid sequence of SEQ ID NO:147.

262. The method of claim 260 or 261, wherein:
(a) the VH of the Fab comprises the amino acid sequence of SEQ ID NO:95, and the VL of the Fab comprises the amino acid sequence of SEQ ID NO:85; and
(b) the scFv comprises the amino acid sequence of SEQ ID NO:148 or SEQ ID NO:149.

263. The method of any one of claims 256 to 262, wherein the multi-specific binding protein is administered as a pharmaceutical formulation comprising:
(a) about 15 mg/mL of the multi-specific binding protein;
(b) about 20 mM citrate;
(c) about 6% (w/v) mannitol; and
(d) about 0.01% (w/v) polysorbate 80,
at about pH 6.5.

264. The method of any one of claims 158 to 263, wherein more than 97% of the multi-specific binding protein has native conformation, as determined by size-exclusion chromatography.

265. The method of any one of claims 158 to 264, wherein less than 2% of the multi-specific binding protein form a high molecular weight complex, as determined by size-exclusion chromatography.

266. The method of any one of claims 158 to 265, wherein the multi-specific binding protein binds human CD16 with a binding affinity (K_{D}) of 48 nM to 160 nM, as measured by surface plasmon resonance (SPR).

267. The method of any one of claims 158 to 266, wherein the multi-specific binding protein binds EGFR with a K_{D} of 4.2 nM to 5.2 nM, as measured by SPR.

268. The method of any one of claims 158 to 267, wherein the multi-specific binding protein binds EGFR with an association rate constant of 1.5×10⁵ to 2.5×10⁵ l/Ms, as measured by SPR.

269. The method of any one of claims 158 to 268, wherein the multi-specific binding protein binds EGFR with a dissociation rate constant of 9.0×10⁻⁴ to 10.0×10⁻⁴ l/s, as measured by SPR.

270. The method of any one of claims 158 to 269, wherein the multi-specific binding protein binds NKG2D with a K_{D} of 4.50×10⁻⁴ mM to 5.20×10⁻⁴ mM, as measured by SPR.

271. The method of any one of claims 158 to 270, wherein the multi-specific binding protein binds NKG2D with an association rate constant of 2.0×10⁵ to 2.6×10⁵ l/Ms, as measured by SPR.

272. The method of any one of claims 158 to 271, wherein the multi-specific binding protein binds EGFR with a dissociation rate constant of 0.6×10⁻¹ to 1.6×10⁻¹ l/s, as measured by SPR.

273. The method of any one of claims 182 to 272, wherein the pharmaceutical formulation is stable at room temperature for at least 1, at least 3, or at least 6 months.

274. The method of any one of claims 182 to 273, wherein the pharmaceutical formulation is stable at −80° C. for at least 1, at least 3, at least 6, at least 9, at least 12, at least 18, or at least 24 months.

275. The method of claim 274, wherein the pharmaceutical formulation is stable at −80° C. for at least 6 months.

276. The method of claim 275, wherein the pharmaceutical formulation is stable at −80° C. for at least 9 months.

277. The method of claim 276, wherein the pharmaceutical formulation is stable at −80° C. for at least 12 months.

278. The method of any one of claims 182 to 277, wherein the pharmaceutical formulation is stable at −20° C. for at least 1, at least 3, at least 6, at least 9 or at least 12 months.

279. The method of claim 278, wherein the pharmaceutical formulation is stable at −20° C. for at least 6 months.

280. The method of any one of claims 182 to 279, wherein the pharmaceutical formulation is stable at −5° C. for at least 1, at least 3, at least 6, at least 9, or at least 12 months.

281. The method of claim 280, wherein the pharmaceutical formulation is stable at −5° C. for at least 6 months.

282. The method of any one of claims 182 to 281, wherein the pharmaceutical formulation is stable at refrigerated temperatures for at least 1, at least 3, at least 6, at least 9, or at least 12 months.

283. The method of any one of claims 158 to 282, wherein the multi-specific binding protein is capable of inhibiting EGFR signaling in EGFR-expressing cancer cells.

284. The method of any one of claims 158 to 283, wherein the multi-specific binding protein is capable of activating NK cell-mediated killing of EGFR-expressing cancer cells.

285. The method of any one of claims 158 to 284, wherein the multi-specific binding protein is capable of activating production and release of one or more chemokines and/or cytokines selected from IFNγ, TNFα, CCL4, CCL5, CXCL9, and CXCL10 from NK cells.

286. The method of any one of claims 158 to 285, wherein the multi-specific binding protein is capable of activating CD8⁺ T cell killing of EGFR-expressing cancer cells.

287. The method of any one of claims 158 to 286, wherein the multi-specific binding protein does not activate CD8⁺ T cells in the periphery.

288. The method of any one of claims 158 to 287, wherein the multi-specific binding protein does not activate CD4⁺ T cells.

289. The method of any one of claims 158 to 288, wherein the multi-specific binding protein is capable of binding human NKG2D and cynomolgus monkey NKG2D.

290. The method of any one of claims 182 to 289, wherein the method is for treating an unresectable solid tumor in the subject.

291. The method of any one of claims 182 to 290, wherein the pharmaceutical formulation is capable of treating a recurrent solid tumor in the subject.

292. The method of any one of claims 182 to 291, wherein the pharmaceutical formulation is capable of treating an advanced solid tumor in the subject for which there is no effective standard therapy.

293. The method of any one of claims 182 to 292, wherein the pharmaceutical formulation is capable of treating subjects intolerant of standard therapies.

294. The method of any one of claims 182 to 293, wherein the pharmaceutical formulation is administered to the subject to achieve a multi-specific binding protein dose of 5 mg/kg to 50 mg/kg.

295. The method of any one of claims 182 to 293, wherein the pharmaceutical formulation is administered to the subject once weekly in one or more 4-week treatment cycles.

296. The method of claim 295, wherein the pharmaceutical formulation is administered to the subject on day 1, day 8, day 15, and day 22 of the one or more 4-week treatment cycles.

297. The method of claim 295 or 296, wherein after a completed 4-week treatment cycle, the pharmaceutical formulation is administered to the subject to achieve an increased dose of the multi-specific binding protein in a subsequent 4-week treatment cycle as compared to the earlier completed 4-week treatment cycle.

298. The method of any one of claims 182 to 297, wherein the pharmaceutical formulation is administered to the subject by intravenous infusion.

299. The method of any one of claims 182 to 298, wherein the pharmaceutical formulation is administered to the subject in combination with an anti-PD-1 or an anti-PD-L1 therapy.

300. The method of claim 299, wherein the anti-PD-1 or anti-PD-L1 therapy is selected from nivolumab, pembrolizumab, durvalumab, or atezolizumab.

301. The method of claim 300, wherein the anti-PD-1 or anti-PD-L1 therapy is nivolumab.

302. The method of claim 301, wherein the nivolumab is administered at about 480 mg.

303. The method of claim 301 or 302, wherein the nivolumab is administered on day 8 of each treatment cycle.

304. The method of claim 300, wherein the anti-PD-1 or anti-PD-L1 therapy is pembrolizumab.

305. The method of claim 304, wherein the pembrolizumab is administered at about 400 mg.

306. The method of claim 304 or 305, wherein the pembrolizumab is administered once every 6 weeks.

307. The method of any one of claims 299 to 306, wherein the subject is eligible for anti-PD-1 or an anti-PD-L1 therapy for a malignancy of epithelial origin.

308. The method of claim 299, wherein no standard therapy exists or standard therapy of the subject has failed for a malignancy of epithelial origin.

309. The method of any one of claims 299 to 308, wherein the subject previously received anti-PD-1 or anti-PD-L1 therapy.

310. The method of any one of claims 158 to 167, 182, or 186 to 309, wherein the cancer is a head and neck squamous cell carcinoma (HNSCC).

311. The method of claim 310, wherein the HNSCC is a relapsed or metastatic HNSCC.

312. The method of claim 310 or 311, wherein the subject has radiographic disease progression while on or after having received:
(i) pembrolizumab and platinum/5FU;
(ii) pembrolizumab monotherapy; or
(iii) platinum/5FU and cetuximab.

313. The method of any one of claims 158 to 167, 182, or 186 to 308, wherein the cancer is a colorectal cancer (CRC).

314. The method of claim 313, wherein the CRC is a relapsed or metastatic CRC.

315. The method of claim 313 or 314, wherein the subject has been treated with FOLFOX, CAPOX, FOLFIRI, or FOLFOXIRI, with or without a biological agent.

316. The method of any one of claims 313 to 315, wherein the subject does not have high mismatch repair/microsatellite instability.

317. The method of any one of claims 313 to 316, wherein the subject has not had prior treatment with an anti-PD-1 or an anti-PD-L1 therapy.

318. The method of any one of claims 313 to 317, wherein the subject has radiographic disease progression while or after receiving treatment for advanced (recurrent/unresectable/metastatic) cancer.

319. The method of any one of claims 158 to 167, 182, or 186 to 308, wherein the cancer is a non-small-cell lung cancer (NSCLC).

320. The method of claim 319, wherein the subject has recurrent or progressive disease during or after platinum doublet-based chemotherapy, or has recurrent or progressive disease within 6 months after completing platinum-based chemotherapy for local disease.

321. The method of claim 319 or 320, wherein the subject has previously received an anti-PD-1 or anti-PD-L1 therapy.

322. The method of any one of claims 158 to 167, 182, or 186 to 309, wherein the cancer is an esophageal adenocarcinoma.

323. The method of any one of claims 158 to 167, 182, or 186 to 309, wherein the cancer is a triple-negative breast cancer.

324. The method of any one of claims 158 to 167, 182, or 186 to 309, wherein the cancer is a renal cell carcinoma.

325. The method of any one of claims 158 to 167, 182, or 186 to 309, wherein the cancer is a gastric cancer.

326. The method of any one of claims 158 to 167, 182, or 186 to 309, wherein the cancer is a pancreatic cancer.

327. The method of any one of claims 182 to 326, wherein the pharmaceutical formulation is administered to the subject in combination with an effective amount of pre-medication comprising: (a) an antihistamine and an antipyretic; or (b) a corticosteroid.

328. The method of claim 182 to 326, wherein the pharmaceutical formulation is administered to the subject in combination with an effective amount of pre-medication comprising: (a) an antihistamine and an antipyretic; and (b) a corticosteroid.

329. The method of claim 328, wherein the antihistamine and antipyretic are administered before each and every infusion of the pharmaceutical formulation, and the corticosteroid is administered before a first dose of a treatment cycle only.

330. The method of any one of claims 327 to 329, wherein the antihistamine is diphenhydramine.

331. The method of any one of claims 327 to 330, wherein the antipyretic is acetaminophen.

332. The method of any one of claims 327 to 331, wherein the corticosteroid is methylprednisolone.

333. The method of claim 332, wherein the methylprednisolone is administered to the subject at about 125 mg.

334. The method of claim 332 or 333, wherein the methylprednisolone is administered to the subject within 60 minutes prior to the first dose of the pharmaceutical formulation.

335. The method of any one of claims 182 to 334, wherein the pharmaceutical formulation is capable of inhibiting EGFR signaling in the subject.

336. The method of any one of claims 182 to 335, wherein the pharmaceutical formulation results in reduced anti-drug antibody (ADA) levels when administered to the subject relative to other anti-EGFR therapeutics.

337. The method of claim 336, wherein the pharmaceutical formulation results in substantially no ADA production when administered to the subject.

338. The method of any one of claims 182 to 337, wherein the pharmaceutical formulation results in reduced toxicity when administered to the subject relative to other anti-EGFR therapeutics.

339. The method of claim 338, wherein toxicity comprises one or more of skin toxicity, keratitis, ulcerative keratitis, corneal perforation, diarrhea, hypomagnesemia, infusion-related reactions, thrombocytopenia, neutropenia, fatigue, hypertension, vomiting, and nausea.

340. The method of any one of claims 158 to 339, wherein the subject is diagnosed as having an EGFR-positive cancer, as determined by immunohistochemistry.

341. The method of any one of claims 158 to 340, wherein the subject is diagnosed as having an EGFR-positive cancer, wherein the cancer has an activating mutation or gene amplification of the EGFR gene.

342. The method of claim 341, wherein EGFR gene amplification is determined by fluorescent in situ hybridization.

343. The method of claim 342, wherein EGFR activating mutation or gene amplification is determined by DNA sequencing.

344. A method of treating cancer in a subject in need thereof, the method comprising administering:
i) an effective amount of pre-medication comprising:
(a) an antihistamine and an antipyretic; or
(b) corticosteroid, and
ii) an effective amount of a multi-specific binding protein comprising:
(i) a Fab that binds NKG2D;
(ii) a single-chain variable fragment (scFv) that binds EGFR, wherein the scFv comprises:
1) a heavy chain variable domain (VH) having complementarity-determining region 1 (CDR1), complementarity-determining region 2 (CDR2), and complementarity-determining region 3 (CDR3) sequences of SEQ ID NOs: 136, 157, and 138, respectively; and a light chain variable domain (VL) having CDR1, CDR2, and CDR3 sequences of SEQ ID NOs: 140, 141, and 151, respectively;
2) a VH having CDR1, CDR2, and CDR3 sequences of SEQ ID NOs: 136, 146, and 138, respectively; and a VL having CDR1, CDR2, and CDR3 sequences of SEQ ID NOs: 140, 141, and 142, respectively; or
3) a VH having CDR1, CDR2, and CDR3 sequences of SEQ ID NOs: 136, 137, and 138, respectively; and a VL having CDR1, CDR2, and CDR3 sequences of SEQ ID NOs: 140, 141, and 142, respectively, and
(iii) an antibody Fc domain, or a portion thereof sufficient to bind CD16, or a third antigen-binding site that binds CD16.

345. A method of treating cancer in a subject in need thereof, the method comprising administering:
i) an effective amount of pre-medication comprising:
(a) an antihistamine and an antipyretic; and
(b) corticosteroid, and
ii) an effective amount of a multi-specific binding protein comprising:
(i) a Fab that binds NKG2D;
(ii) a single-chain variable fragment (scFv) that binds EGFR, wherein the scFv comprises:
1) a heavy chain variable domain (VH) having complementarity-determining region 1 (CDR1), complementarity-determining region 2 (CDR2), and complementarity-determining region 3 (CDR3) sequences of SEQ ID NOs: 136, 157, and 138, respectively; and a light chain variable domain (VL) having CDR1, CDR2, and CDR3 sequences of SEQ ID NOs: 140, 141, and 151, respectively;
2) a VH having CDR1, CDR2, and CDR3 sequences of SEQ ID NOs: 136, 146, and 138, respectively; and a VL having CDR1, CDR2, and CDR3 sequences of SEQ ID NOs: 140, 141, and 142, respectively; or
3) a VH having CDR1, CDR2, and CDR3 sequences of SEQ ID NOs: 136, 137, and 138, respectively; and a VL having CDR1, CDR2, and CDR3 sequences of SEQ ID NOs: 140, 141, and 142, respectively, and
(iii) an antibody Fc domain, or a portion thereof sufficient to bind CD16, or a third antigen-binding site that binds CD16.

346. The method of claim 344 or 345, wherein the multi-specific binding protein is administered as a pharmaceutical formulation comprising one or more of:
(a) citrate;
(b) a sugar or sugar alcohol; and
(c) a polysorbate
at pH 6.0 to 7.0.

347. The method of claim 346, wherein the concentration of the multi-specific binding protein in the pharmaceutical formulation is 1 mg/mL to 125 mg/mL.

348. The method of claim 346 or 347, wherein the concentration of the multi-specific binding protein in the pharmaceutical formulation is 2 mg/mL to 100 mg/mL.

349. The method of any one of claims 346 to 348, wherein the concentration of the multi-specific binding protein in the pharmaceutical formulation is 5 mg/mL to 50 mg/mL.

350. The method of any one of claims 346 to 349, wherein the concentration of the multi-specific binding protein in the pharmaceutical formulation is 5 mg/mL to 20 mg/mL.

351. The method of any one of claims 346 to 350, wherein the concentration of the multi-specific binding protein in the pharmaceutical formulation is 10 mg/mL to 20 mg/mL.

352. The method of any one of claims 346 to 351, wherein the concentration of the multi-specific binding protein in the pharmaceutical formulation is about 15 mg/mL.

353. The method of any one of claims 346 to 352, wherein the formulation is diluted with a suitable diluent in the range of 1:0 to 1:10 prior to administration to a subject.

354. The method of any one of claims 346 to 353, wherein the concentration of citrate in the pharmaceutical formulation is 15 mM to 25 mM.

355. The method of any one of claims 346 to 354, wherein the concentration of citrate in the pharmaceutical formulation is 17.5 mM to 22.5 mM.

356. The method of any one of claims 346 to 355, wherein the concentration of citrate in the pharmaceutical formulation is about 20 mM.

357. The method of any one of claims 346 to 356, wherein the sugar alcohol is an alcohol of a monosaccharide.

358. The method of any one of claims 346 to 357, wherein the sugar alcohol is mannitol.

359. The method of claim 358, wherein the concentration of mannitol is 4% to 8% (w/v).

360. The method of claim 358 or 359, wherein the concentration of mannitol is 5% to 7% (w/v).

361. The method of claim 360, wherein the concentration of mannitol is about 6% (w/v).

362. The method of any one of claims 346 to 361, wherein the polysorbate is polysorbate 80.

363. The method of claim 362, wherein the concentration of polysorbate 80 is 0.005% to 0.05% (w/v).

364. The method of claim 362 or 363363, wherein the concentration of polysorbate 80 is about 0.0075% to 0.025% (w/v).

365. The method of any one of claims 362 to 364, wherein the concentration of polysorbate 80 is about 0.01% (w/v).

366. The method of any one of claims 346 to 365, wherein the pH is 6.2 to 6.8.

367. The method of any one of claims 346 to 366, wherein the pH is 6.4 to 6.6.

368. The method of any one of claims 346 to 367, wherein the pH is about 6.5.

369. The method of any one of claims 346 to 349, 353, 354, 357 to 359, 362, 363, or 366, wherein the formulation comprises:
(a) 5 mg/mL to 50 mg/mL of the multi-specific binding protein;
(b) 15 mM to 25 mM citrate;
(c) 4% to 8% (w/v) mannitol; and
(d) 0.005% to 0.05% (w/v) polysorbate 80,
at pH 6.2 to 6.8.

370. The method of any one of claims 346 to 351, 353 to 355, 357 to 360, 362 to 366, 368, or 369, wherein the formulation comprises:
(a) 10 mg/mL to 20 mg/mL of the multi-specific binding protein;
(b) 17.5 mM to 22.5 mM citrate;
(c) 5% to 7% (w/v) mannitol; and
(d) 0.0075% to 0.025% (w/v) polysorbate 80,
at pH 6.4 to 6.6.

371. The method of any one of claims 346 to 370, wherein the formulation comprises:
(a) about 15 mg/mL of the multi-specific binding protein;
(b) about 20 mM citrate;
(c) about 6% (w/v) mannitol; and
(d) about 0.01% (w/v) polysorbate 80,
at about pH 6.5.

372. The method of any one of claims 344 to 371, wherein the VL of the scFv is linked to the VH of the scFv via a flexible linker.

373. The method of claim 372, wherein the flexible linker comprises the amino acid sequence of SEQ ID NO:119.

374. The method of claim 372 or 373, wherein the flexible linker consists of the amino acid sequence of SEQ ID NO:119.

375. The method of any one of claims 344 to 374, wherein the VL of the scFv is positioned to the N-terminus of the VH of the scFv, or the VH of the scFv is positioned to the N-terminus of the VL of the scFv.

376. The method of any one of claims 344 to 375, wherein the VH of the scFv forms a disulfide bridge with the VL of the scFv.

377. The method of claim 376, wherein the disulfide bridge is formed between C44 of the VH of the scFv and C100 of the VL of the scFv, numbered under the Kabat numbering scheme.

378. The method of any one of claims 344 to 377, wherein the antibody Fc domain comprises a first antibody Fc polypeptide linked to the Fab and a second antibody Fc polypeptide linked to the scFv.

379. The method of claim 378, wherein the first antibody Fc polypeptide is linked to a heavy chain portion of the Fab.

380. The method of claim 378 or 379, wherein the scFv is linked to the second antibody Fc polypeptide via a hinge comprising Ala-Ser or Gly-Ser.

381. The method of any one of claims 378 to 380, wherein the first and second antibody Fc polypeptides each comprise a hinge and a CH2 domain of a human IgG1 antibody.

382. The method of claim 381, wherein the first and second antibody Fc polypeptides each comprise an amino acid sequence at least 90% identical to amino acids 234-332 of a wild-type human IgG1 antibody, numbered according to the EU index.

383. The method of any one of claims 378 to 382, wherein the first and second antibody Fc polypeptides each comprise different mutations promoting heterodimerization.

384. The method of claim 383, wherein the first antibody Fc polypeptide is a human IgG1 Fc polypeptide comprising K360E and K409W substitutions, numbered according to the EU index.

385. The method of claim 383 or 384, wherein the second antibody Fc polypeptide is a human IgG1 Fc polypeptide comprising Q347R, D399V, and F405T substitutions, numbered according to the EU index.

386. The method of any one of claims 344 to 385, wherein the Fab comprises:
(a) a VH comprising CDR1, CDR2, and CDR3 sequences of SEQ ID NOs: 81, 82, and 112, respectively; and
(b) a VL comprising CDR1, CDR2, and CDR3 sequences of SEQ ID NOs: 86, 77, and 87, respectively.

387. The method of claim 386, wherein the Fab comprises:
(a) a VH comprising CDR1, CDR2, and CDR3 sequences of SEQ ID NOs: 81, 82, and 97, respectively; and
(b) a VL comprising CDR1, CDR2, and CDR3 sequences of SEQ ID NOs: 86, 77, and 87, respectively.

388. The method of claim 386 or 387, wherein the VH of the Fab comprises an amino acid sequence at least 90% identical to SEQ ID NO:95 or at least 90% identical to SEQ ID NO:110, and the VL of the Fab comprises an amino acid sequence at least 90% identical to SEQ ID NO:85.

389. The method of any one of claims 386 to 388, wherein the VH of the Fab comprises the amino acid sequence of SEQ ID NO:95, and the VL of the Fab comprises the amino acid sequence of SEQ ID NO:85.

390. The method of any one of claims 344 to 389, wherein the VH of the scFv comprises CDR1, CDR2, and CDR3 sequences of SEQ ID NOs: 136, 137, and 138, respectively; and the VL of the scFv comprises CDR1, CDR2, and CDR3 sequences of SEQ ID NOs: 140, 141, and 142, respectively.

391. The method of any one of claims 344 to 390, wherein the VH of the scFv comprises an amino acid sequence at least 90% identical to SEQ ID NO:135, and the VL of the scFv comprises an amino acid sequence at least 90% identical to SEQ ID NO:139.

392. The method of any one of claims 344 to 391, wherein the VH of the scFv comprises the amino acid sequence of SEQ ID NO:135, wherein the glycine at position 44 of the VH is substituted with a cysteine (C44), numbered under the Kabat numbering scheme, and the VL of the scFv comprises the amino acid sequence of SEQ ID NO:139, wherein the glycine at position 100 of the VL is substituted with a cysteine (C100), numbered under the Kabat numbering scheme, and wherein the C44 and the C100 form a disulfide bond between the VH and the VL.

393. The method of any one of claims 344 to 392, wherein the scFv comprises the amino acid sequence of SEQ ID NO:143 or SEQ ID NO:144.

394. The method of any one of claims 344 to 377, wherein:
(a) the Fab comprises a VH comprising CDR1, CDR2, and CDR3 sequences of SEQ ID NOs: 81, 82, and 97, respectively; and a VL comprising CDR1, CDR2, and CDR3 sequences of SEQ ID NOs: 86, 77, and 87, respectively;
(b) the VH of the scFv comprises CDR1, CDR2, and CDR3 sequences of SEQ ID NOs: 136, 137, and 138, respectively; and the VL of the scFv comprises CDR1, CDR2, and CDR3 sequences of SEQ ID NOs: 140, 141, and 142, respectively; and
(c) the antibody Fc domain comprises a first antibody Fc polypeptide linked to the Fab and a second antibody Fc polypeptide linked to the scFv, wherein the first antibody Fc polypeptide is a human IgG1 Fc polypeptide comprising K360E and K409W substitutions, and the second antibody Fc polypeptide is a human IgG1 Fc polypeptide comprising Q347R, D399V, and F405T substitutions, numbered according to the EU index.

395. The method of claim 394, wherein:
(a) the VH of the Fab comprises an amino acid sequence at least 90% identical to SEQ ID NO:95 or an amino acid sequence at least 90% identical to SEQ ID NO:110, and the VL of the Fab comprises an amino acid sequence at least 90% identical to SEQ ID NO:85; and
(b) the VH of the scFv comprises an amino acid sequence at least 90% identical to SEQ ID NO:135, and the VL of the scFv comprises an amino acid sequence at least 90% identical to SEQ ID NO:139.

396. The method of claim 395, wherein the scFv comprises the amino acid sequence of SEQ ID NO:143 or SEQ ID NO:144.

397. The method of any one of claims 390 to 396, wherein the pharmaceutical formulation comprises:
(a) about 15 mg/mL of the multi-specific binding protein;
(b) about 20 mM citrate;
(c) about 6% (w/v) mannitol; and
(d) about 0.01% (w/v) polysorbate 80,
at about pH 6.5.

398. The method of any one of claims 344 to 389, wherein the VH of the scFv comprises CDR1, CDR2, and CDR3 sequences of SEQ ID NOs: 136, 157, and 138, respectively; and the VL of the scFv comprises CDR1, CDR2, and CDR3 sequences of SEQ ID NOs: 140, 141, and 151, respectively.

399. The method of any one of claims 344 to 389, or 398 wherein the VH of the scFv comprises an amino acid sequence at least 90% identical to SEQ ID NO:156, and the VL of the scFv comprises an amino acid sequence at least 90% identical to SEQ ID NO:150.

400. The method of any one of claims 344 to 389, 398, or 399, wherein the scFv comprises the amino acid sequence of SEQ ID NO:158 or SEQ ID NO:159.

401. The method of any one of claims 344 to 389, wherein the VH of the scFv comprises CDR1, CDR2, and CDR3 sequences of SEQ ID NOs: 136, 146, and 138, respectively; and the VL of the scFv comprises CDR1, CDR2, and CDR3 sequences of SEQ ID NOs: 140, 141, and 151, respectively.

402. The method of any one of claims 344 to 389, or 401, wherein the VH of the scFv comprises an amino acid sequence at least 90% identical to SEQ ID NO:170 or at least 90% identical to SEQ ID NO:145, and the VL of the scFv comprising an amino acid sequence at least 90% identical to SEQ ID NO:171 or at least 90% identical to SEQ ID NO:150.

403. The method of any one of claims 344 to 389, 401, or 402, wherein the VH of the scFv comprises the amino acid sequence of SEQ ID NO:170, and the VL of the scFv comprises the amino acid sequence of SEQ ID NO:171.

404. The method of any one of claims 344 to 389, or 401 to 403, wherein the scFv comprises the amino acid sequence of SEQ ID NO:152 or SEQ ID NO:153.

405. The method of any one of claims 344 to 377, wherein:
(a) the Fab comprises a VH comprising CDR1, CDR2, and CDR3 sequences of SEQ ID NOs: 81, 82, and 97, respectively; and a VL comprising CDR1, CDR2, and CDR3 sequences of SEQ ID NOs: 86, 77, and 87, respectively;
(b) the VH of the scFv comprises CDR1, CDR2, and CDR3 sequences of SEQ ID NOs: 136, 146, and 138, respectively; and the VL of the scFv comprises CDR1, CDR2, and CDR3 sequences of SEQ ID NOs: 140, 141, and 151, respectively; and
(c) the antibody Fc domain comprises a first antibody Fc polypeptide linked to the Fab and a second antibody Fc polypeptide linked to the scFv, wherein the first antibody Fc polypeptide is a human IgG1 Fc polypeptide comprising K360E and K409W substitutions, and the second antibody Fc polypeptide is a human IgG1 Fc polypeptide comprising Q347R, D399V, and F405T substitutions, numbered according to the EU index.

406. The method of claim 405, wherein:
(a) the VH of the Fab comprises an amino acid sequence at least 90% identical to SEQ ID NO:95 or an amino acid sequence at least 90% identical to SEQ ID NO:110, and the VL of the Fab comprises an amino acid sequence at least 90% identical to SEQ ID NO:85;
(b) the VH of the scFv comprises the amino acid sequence of SEQ ID NO:170, and the VL of the scFv comprises the amino acid sequence of SEQ ID NO:171; and
(c) the antibody Fc domain comprises a first antibody Fc polypeptide linked to the Fab and a second antibody Fc polypeptide linked to the scFv, wherein the first antibody Fc polypeptide is a human IgG1 Fc polypeptide comprising K360E and K409W substitutions, and the second antibody Fc polypeptide is a human IgG1 Fc polypeptide comprising Q347R, D399V, and F405T substitutions, numbered according to the EU index.

407. The method of claim 406, wherein the scFv comprises the amino acid sequence of SEQ ID NO:152 or SEQ ID NO:153.

408. The method of any one of claims 401 to 407, wherein the multi-specific binding protein comprises:
(a) a first polypeptide comprising the amino acid sequence of SEQ ID NO:167;
(b) a second polypeptide comprising the amino acid sequence of SEQ ID NO:164; and
(c) a third polypeptide comprising the amino acid sequence of SEQ ID NO:165.

409. The method of any one of claims 401 to 408, wherein the pharmaceutical formulation comprises:
(a) about 15 mg/mL of the multi-specific binding protein;
(b) about 20 mM citrate;
(c) about 6% (w/v) mannitol; and
(d) about 0.01% (w/v) polysorbate 80,
at about pH 6.5.

410. The method of any one of claims 344 to 389, wherein the VH of the scFv comprises CDR1, CDR2, and CDR3 sequences of SEQ ID NOs: 136, 137, and 138, respectively; and the VL of the scFv comprises CDR1, CDR2, and CDR3 sequences of SEQ ID NOs: 140, 141, and 151, respectively.

411. The method of any one of claims 344 to 389, or 410, wherein the VH of the scFv comprises an amino acid sequence at least 90% identical to SEQ ID NO:135, and the VL of the scFv comprises an amino acid sequence at least 90% identical to SEQ ID NO:150.

412. The method of any one of claims 344 to 389, 410, or 411, wherein the VH of the scFv comprises the amino acid sequence of SEQ ID NO:135, wherein the glycine at position 44 of the VH is substituted with a cysteine (C44), numbered under the Kabat numbering scheme, and the VL of the scFv comprises the amino acid sequence of SEQ ID NO:150, wherein the glycine at position 100 of the VL is substituted with a cysteine (C100), numbered under the Kabat numbering scheme, and wherein the C44 and the C100 form a disulfide bond between the VH and the VL.

413. The method of any one of claims 344 to 389, or 410 to 412, wherein the scFv comprises the amino acid sequence of SEQ ID NO:154 or SEQ ID NO:155.

414. The method of any one of claims 344 to 377, wherein:
(a) the Fab comprises a VH comprising CDR1, CDR2, and CDR3 sequences of SEQ ID NOs: 81, 82, and 97, respectively; and a VL comprising CDR1, CDR2, and CDR3 sequences of SEQ ID NOs: 86, 77, and 87, respectively;
(b) the VH of the scFv comprises CDR1, CDR2, and CDR3 sequences of SEQ ID NOs: 136, 137, and 138, respectively; and the VL of the scFv comprises CDR1, CDR2, and CDR3 sequences of SEQ ID NOs: 140, 141, and 151, respectively; and
(c) the antibody Fc domain comprises a first antibody Fc polypeptide linked to the Fab and a second antibody Fc polypeptide linked to the scFv, wherein the first antibody Fc polypeptide is a human IgG1 Fc polypeptide comprising K360E and K409W substitutions, and the second antibody Fc polypeptide is a human IgG1 Fc polypeptide comprising Q347R, D399V, and F405T substitutions, numbered according to the EU index.

415. The method of claim 414, wherein:
(a) the VH of the Fab comprises an amino acid sequence at least 90% identical to SEQ ID NO:95 or an amino acid sequence at least 90% identical to SEQ ID NO:110, and the VL of the Fab comprises an amino acid sequence at least 90% identical to SEQ ID NO:85; and
(b) the VH of the scFv comprises an amino acid sequence at least 90% identical to SEQ ID NO:135, and the VL of the scFv comprises an amino acid sequence at least 90% identical to SEQ ID NO:150.

416. The method of claim 415, wherein the scFv comprises the amino acid sequence of SEQ ID NO:154 or SEQ ID NO:155.

417. The method of any one of claims 410 to 416, wherein the pharmaceutical formulation comprises:
(a) about 15 mg/mL of the multi-specific binding protein;
(b) about 20 mM citrate;
(c) about 6% (w/v) mannitol; and
(d) about 0.01% (w/v) polysorbate 80,
at about pH 6.5.

418. The method of any one of claims 344 to 389, wherein the VH of the scFv comprises CDR1, CDR2, and CDR3 sequences of SEQ ID NOs: 136, 146, and 138, respectively; and the VL of the scFv comprises CDR1, CDR2, and CDR3 sequences of SEQ ID NOs: 140, 141, and 142, respectively.

419. The method of any one of claims 344 to 389, or 418, wherein the VH of the scFv comprises an amino acid sequence at least 90% identical to SEQ ID NO:145, and the VL of the scFv comprises an amino acid sequence at least 90% identical to SEQ ID NO:147.

420. The method of any one of claims 344 to 389, 418, or 419, wherein the VH of the scFv comprises the amino acid sequence of SEQ ID NO:145, wherein the glycine at position 44 of the VH is substituted with a cysteine (C44), numbered under the Kabat numbering scheme, and the VL of the scFv comprises the amino acid sequence of SEQ ID NO:147, wherein the glycine at position 100 of the VL is substituted with a cysteine (C100), numbered under the Kabat numbering scheme, and wherein the C44 and the C100 form a disulfide bond between the VH and VL.

421. The method of any one of claims 344 to 389, or 418 to 420, wherein the scFv comprises the amino acid sequence of SEQ ID NO:148 or SEQ ID NO:149.

422. The method of any one of claims 344 to 377, wherein:
(a) the Fab comprises a VH comprising CDR1, CDR2, and CDR3 sequences of SEQ ID NOs: 81, 82, and 97, respectively; and a VL comprising CDR1, CDR2, and CDR3 sequences of SEQ ID NOs: 86, 77, and 87, respectively;
(b) the VH of the scFv comprises CDR1, CDR2, and CDR3 sequences of SEQ ID NOs: 136, 146, and 138, respectively; and the VL of the scFv comprises CDR1, CDR2, and CDR3 sequences of SEQ ID NOs: 140, 141, and 142, respectively; and
(c) the antibody Fc domain comprises a first antibody Fc polypeptide linked to the Fab and a second antibody Fc polypeptide linked to the scFv, wherein the first antibody Fc polypeptide is a human IgG1 Fc polypeptide comprising K360E and K409W substitutions, and the second antibody Fc polypeptide is a human IgG1 Fc polypeptide comprising Q347R, D399V, and F405T substitutions, numbered according to the EU index.

423. The method of claim 422, wherein:
(a) the VH of the Fab comprises an amino acid sequence at least 90% identical to SEQ ID NO:95 or an amino acid sequence at least 90% identical to SEQ ID NO:110, and the VL of the Fab comprises an amino acid sequence at least 90% identical to SEQ ID NO:85; and
(b) the VH of the scFv comprises an amino acid sequence at least 90% identical to the amino acid sequence of SEQ ID NO:145, and the VL of the scFv comprises an amino acid sequence at least 90% identical to the amino acid sequence of SEQ ID NO:147.

424. The method of claim 423, wherein the scFv comprises the amino acid sequence of SEQ ID NO:148 or SEQ ID NO:149.

425. The method of any one of claims 418 to 424, wherein the pharmaceutical formulation comprises:
(a) about 15 mg/mL of the multi-specific binding protein;
(b) about 20 mM citrate;
(c) about 6% (w/v) mannitol; and
(d) about 0.01% (w/v) polysorbate 80,
at about pH 6.5.

426. The method of any one of claims 344 to 425, wherein more than 97% of the multi-specific binding protein has native conformation, as determined by size-exclusion chromatography.

427. The method of any one of claims 344 to 426, wherein less than 2% of the multi-specific binding protein form a high molecular weight complex, as determined by size-exclusion chromatography.

428. The method of any one of claims 344 to 427, wherein the multi-specific binding protein binds human CD16 with a binding affinity (K_{D}) of 48 nM to 160 nM, as measured by surface plasmon resonance (SPR).

429. The method of any one of claims 344 to 428, wherein the multi-specific binding protein binds EGFR with a K_{D} of 4.2 nM to 5.2 nM, as measured by SPR.

430. The method of any one of claims 344 to 429, wherein the multi-specific binding protein binds EGFR with an association rate constant of 1.5×10⁵ to 2.5×10⁵ l/Ms, as measured by SPR.

431. The method of any one of claims 344 to 430, wherein the multi-specific binding protein binds EGFR with a dissociation rate constant of 9.0×10⁻⁴ to 10.0×10⁻⁴ l/s, as measured by SPR.

432. The method of any one of claims 344 to 431, wherein the multi-specific binding protein binds NKG2D with a K_{D} of 4.50×10⁻⁴ mM to 5.20×10⁻⁴ mM, as measured by SPR.

433. The method of any one of claims 344 to 432, wherein the multi-specific binding protein binds NKG2D with an association rate constant of 2.0×10⁵ to 2.6×10⁵ l/Ms, as measured by SPR.

434. The method of any one of claims 344 to 433, wherein the multi-specific binding protein binds EGFR with a dissociation rate constant of 0.6×10⁻¹ to 1.6×10⁻¹ l/s, as measured by SPR.

435. The method of any one of claims 346 to 434, wherein the pharmaceutical formulation is stable at room temperature for at least 1, at least 3, or at least 6 months.

436. The method of any one of claims 346 to 435, wherein the pharmaceutical formulation is stable at −80° C. for at least 1, at least 3, at least 6, at least 9, at least 12, at least 18, or at least 24 months.

437. The method of claim 436, wherein the pharmaceutical formulation is stable at −80° C. for at least 6 months.

438. The method of claim 437, wherein the pharmaceutical formulation is stable at −80° C. for at least 9 months.

439. The method of claim 438, wherein the pharmaceutical formulation is stable at −80° C. for at least 12 months.

440. The method of any one of claims 346 to 439, wherein the pharmaceutical formulation is stable at −20° C. for at least 1, at least 3, at least 6, at least 9, or at least 12 months.

441. The method of claim 440, wherein the pharmaceutical formulation is stable at −20° C. for at least 6 months.

442. The method of any one of claims 346 to 441, wherein the pharmaceutical formulation is stable at −5° C. for at least 1, at least 3, at least 6, at least 9, or at least 12 months.

443. The method of claim 442, wherein the pharmaceutical formulation is stable at −5° C. for at least 6 months.

444. The method of any one of claims 346 to 443, wherein the pharmaceutical formulation is stable at refrigerated temperatures for at least 1, at least 3, at least 6, at least 9, or at least 12 months.

445. The method of any one of claims 344 to 444, wherein the multi-specific binding protein is capable of inhibiting EGFR signaling in EGFR-expressing cancer cells.

446. The method of any one of claims 344 to 445, wherein the multi-specific binding protein is capable of activating NK cell-mediated killing of EGFR-expressing cancer cells.

447. The method of any one of claims 344 to 446, wherein the multi-specific binding protein is capable of activating production and release of one or more chemokines and/or cytokines selected from IFNγ, TNFα, CCL4, CCL5, CXCL9, and CXCL10 from NK cells.

448. The method of any one of claims 344 to 447, wherein the multi-specific binding protein is capable of activating CD8⁺ T cell killing of EGFR-expressing cancer cells.

449. The method of any one of claims 344 to 448, wherein the multi-specific binding protein does not activate CD8⁺ T cells in the periphery.

450. The method of any one of claims 344 to 449, wherein the multi-specific binding protein does not activate CD4⁺ T cells.

451. The method of any one of claims 344 to 450, wherein the multi-specific binding protein is capable of binding human NKG2D and cynomolgus monkey NKG2D.

452. The method of any one of claims 346 to 451, wherein the pharmaceutical formulation is capable of treating an unresectable solid tumor in the subject.

453. The method of any one of claims 346 to 452, wherein the pharmaceutical formulation is capable of treating a recurrent solid tumor in a subject.

454. The method of any one of claims 346 to 453, wherein the pharmaceutical formulation is capable of treating an advanced solid tumor in a subject for which there is no effective standard therapy.

455. The method of any one of claims 346 to 454, wherein the pharmaceutical formulation is capable of treating subjects intolerant of standard therapies.

456. The method of any one of claims 346 to 455, wherein the pharmaceutical formulation is administered to the subject to achieve a multi-specific binding protein dose of 5 mg/kg to 50 mg/kg.

457. The method of any one of claims 346 to 456, wherein the pharmaceutical formulation is administered once weekly in one or more 4-week treatment cycles.

458. The method of claim 457, wherein the pharmaceutical formulation is administered to the subject on day 1, day 8, day 15, and day 22 of the one or more 4-week treatment cycles.

459. The method of claim 457 or 458, wherein after a completed 4-week treatment cycle, the pharmaceutical formulation is administered to the subject to achieve an increased dose of the multi-specific binding protein in a subsequent 4-week treatment cycle as compared to the earlier completed 4-week treatment cycle.

460. The method of any one of claims 346 to 459, wherein the pharmaceutical formulation is administered to the subject by intravenous infusion.

461. The method of any one of claims 346 to 460, wherein the pharmaceutical formulation is administered to the subject in combination with an anti-PD-1 or an anti-PD-L1 therapy.

462. The method of claim 461, wherein the anti-PD-1 or anti-PD-L1 therapy is selected from nivolumab, pembrolizumab, durvalumab, or atezolizumab.

463. The method of claim 462, wherein the anti-PD-1 or anti-PD-L1 therapy is nivolumab.

464. The method of claim 463, wherein the nivolumab is administered at about 480 mg.

465. The method of claim 462 or 463, wherein the nivolumab is administered on day 8 of each treatment cycle.

466. The method of claim 462, wherein the anti-PD-1 or anti-PD-L1 therapy is pembrolizumab.

467. The method of claim 466, wherein the pembrolizumab is administered at about 400 mg.

468. The method of claim 466 or 467, wherein the pembrolizumab is administered once every 6 weeks.

469. The method of any one of claims 461 to 468, wherein the subject is eligible for anti-PD-1 or an anti-PD-L1 therapy for a malignancy of epithelial origin.

470. The method of claim 461, wherein no standard therapy exists or standard therapy of the subject has failed for a malignancy of epithelial origin.

471. The method of any one of claims 461 to 470, wherein the subject previously received anti-PD-1 or anti-PD-L1 therapy.

472. The method of any one of claims 344 to 471, wherein the cancer is a head and neck squamous cell carcinoma (HNSCC).

473. The method of claim 472, wherein the HNSCC is a relapsed or metastatic HNSCC.

474. The method of claim 472 or 473, wherein the subject has radiographic disease progression while on or after having received:
(i) pembrolizumab and platinum/5FU;
(ii) pembrolizumab monotherapy; or
(iii) platinum/5FU and cetuximab.

475. The method of any one of claims 344 to 470, wherein the cancer is a colorectal cancer (CRC).

476. The method of claim 475, wherein the CRC is a relapsed or metastatic CRC.

477. The method of claim 475 or 476, wherein the subject has been treated with FOLFOX, CAPOX, FOLFIRI, or FOLFOXIRI, with or without a biological agent.

478. The method of any one of claims 475 to 477, wherein the subject does not have high mismatch repair/microsatellite instability.

479. The method of any one of claims 475 to 478, wherein the subject has not had prior treatment with an anti-PD-1 or an anti-PD-L1 therapy.

480. The method of any one of claims 475 to 479, wherein the subject has radiographic disease progression while or after receiving treatment for advanced (recurrent/unresectable/metastatic) cancer.

481. The method of any one of claims 344 to 470, wherein the cancer is a non-small-cell lung cancer (NSCLC).

482. The method of claim 481, wherein the subject has recurrent or progressive disease during or after platinum doublet-based chemotherapy, or has recurrent or progressive disease within 6 months after completing platinum-based chemotherapy for local disease.

483. The method of claim 481 or 482, wherein the subject has previously received an anti-PD-1 or anti-PD-L1 therapy.

484. The method of any one of claims 344 to 471 wherein the cancer is an esophageal adenocarcinoma.

485. The method of any one of claims 344 to 471, wherein the cancer is a triple-negative breast cancer.

486. The method of any one of claims 344 to 471, wherein the cancer is a renal cell carcinoma.

487. The method of any one of claims 344 to 471, wherein the cancer is a gastric cancer.

488. The method of any one of claims 344 to 471, wherein the cancer is a pancreatic cancer.

489. The method of any one of claims 345 to 488, wherein the antihistamine and antipyretic are administered before each and every infusion of the pharmaceutical formulation, and the corticosteroid is administered before a first dose of a treatment cycle only.

490. The method of claim 344 to 489, wherein the antihistamine is diphenhydramine.

491. The method of claim 344 to 490, wherein the antipyretic is acetaminophen.

492. The method of claim 344 to 491, wherein the corticosteroid is methylprednisolone.

493. The method of claim 492, wherein the methylprednisolone is administered at about 125 mg.

494. The method of claim 492 or 493, wherein the methylprednisolone is administered within 60 minutes prior to the first dose of the pharmaceutical formulation.

495. The method of any one of claims 344 to 494, wherein the multi-specific binding protein is capable of inhibiting EGFR signaling in the subject.

496. The method of any one of claims 344 to 495, wherein the multi-specific binding protein results in reduced anti-drug antibody (ADA) levels when administered to the subject relative to other anti-EGFR therapeutics.

497. The method of claim 496, wherein the multi-specific binding protein results in substantially no ADA production when administered to the subject.

498. The method of any one of claims 344 to 497, wherein the multi-specific binding protein results in reduced toxicity when administered to the subject relative to other anti-EGFR therapeutics.

499. The method of claim 498, wherein toxicity comprises one or more of skin toxicity, keratitis, ulcerative keratitis, corneal perforation, diarrhea, hypomagnesemia, infusion-related reactions, thrombocytopenia, neutropenia, fatigue, hypertension, vomiting, and nausea.

500. The method of any one of claims 344 to 499, wherein the subject is diagnosed as having an EGFR-positive cancer, as determined by immunohistochemistry.

501. The method of any one of claims 344 to 500, wherein the subject is diagnosed as having an EGFR-positive cancer, wherein the cancer has an activating mutation or gene amplification of the EGFR gene.

502. The method of claim 501, wherein EGFR gene amplification is determined by fluorescent in situ hybridization.

503. The method of claim 501, wherein EGFR activating mutation or gene amplification is determined by DNA sequencing.

504. A method of purifying a multi-specific protein comprising one or more steps selected from:
a) a protein A affinity purification;
b) a low pH viral inactivation;
c) a mix-mode anion exchange chromatography;
d) a mixed-mode chromatography;
e) a viral filtration; and
f) and ultrafiltration/diafiltration,
wherein the multi-specific binding protein comprises:
(i) a first antigen-binding site that binds NKG2D;
(ii) a second antigen-binding site that binds EGFR; and
(iii) an antibody Fc domain or a portion thereof sufficient to bind CD16, or a third antigen-binding site that binds CD16.

505. The method of claim 504, wherein the protein A affinity purification step comprises:
(a) binding the multi-specific binding protein to a protein A resin; and
(b) eluting the bound multi-specific binding protein at a pH of 3.5-3.8, thereby producing a first eluate.

506. The method of claim 505, wherein the bound multi-specific binding protein is eluted at a pH of about 3.7.

507. The method of claim 505 or 506, wherein the pH of the first eluate is 4.0-4.6.

508. The method of any one of claims 505 to 507, wherein the pH of the first eluate is about 4.3.

509. The method of any one of claims 505 to 508, wherein the low pH viral inactivation step comprises:
(a) addition of an amount of acetic acid to the first eluate sufficient to achieve a pH of 3.5-3.7, thereby producing the viral inactivation reaction mixture;
(b) incubation of the viral inactivation reaction mixture for 30-60 minutes.

510. The method of claim 509, wherein the pH of the low pH viral inactivation reaction mixture is about 3.6.

511. The method of claim 509 or 510, wherein the method comprises addition of an amount of Tris sufficient to bring the pH of the low pH viral inactivation reaction mixture to a pH of 6.3-6.5 following the incubation.

512. The method of any one of claims 504 to 511, wherein the mix-mode anion exchange chromatography step comprises flowing the viral inactivation reaction mixture through an anion exchange column, thereby producing a second eluate.

513. The method of claim 512, wherein the mixed-mode chromatography step comprises:
(a) flowing the second eluate through a ceramic hydroxyapatite (CHT), type I column; and
(b) eluting the multi-specific binding protein from the CHT column in a buffer comprising about 200 mM sodium chloride, thereby producing a third eluate.

514. The method of any one of claims 504 to 513, wherein the method further comprises a buffer exchange step of the multi-specific binding protein to achieve a final buffer concentration of 15 mM to 25 mM citrate, 4% to 8% (w/v) mannitol, and 0.005% to 0.05% (w/v) polysorbate 80, at pH 6.2 to 6.8.

515. The method of any one of claims 504 to 514, wherein the method further comprises a buffer exchange step of the multi-specific binding protein to achieve a final buffer concentration of about 20 mM citrate, about 6% (w/v) mannitol, and about 0.01% (w/v) polysorbate 80, at about pH 6.5.

516. The method of claim 514 or 515, wherein the concentration of the multi-specific binding protein is 1 mg/mL to 125 mg/mL following the buffer exchange step.

517. The method of claim 516, wherein the concentration of the multi-specific binding protein is about 50 mg/mL following the buffer exchange step.

518. The method of any one of claims 504 to 517, wherein:
(a) the first antigen-binding site comprises a Fab comprising a VH comprising CDR1, CDR2, and CDR3 sequences of SEQ ID NOs: 81, 82, and 112, respectively, and a VL comprising CDR1, CDR2, and CDR3 sequences of SEQ ID NOs: 86, 77, and 87, respectively; and
(b) the second antigen-binding site comprises an scFv comprising: (1) a VH comprising CDR1, CDR2, and CDR3 sequences of SEQ ID NOs: 136, 157, and 138, respectively, and a VL comprising CDR1, CDR2, and CDR3 sequences of SEQ ID NOs: 140, 141, and 151, respectively, or (2) a VH having CDR1, CDR2, and CDR3 sequences of SEQ ID NOs: 136, 146, and 138, respectively, and a VL having CDR1, CDR2, and CDR3 sequences of SEQ ID NOs: 140, 141, and 142, respectively.

519. The method of claim 518, wherein:
(a) the VH of the Fab comprises CDR1, CDR2, and CDR3 sequences of SEQ ID NOs: 81, 82, and 97, respectively, and the VL of the Fab comprises CDR1, CDR2, and CDR3 sequences of SEQ ID NOs: 86, 77, and 87, respectively;
(b) the VH of the scFv comprises CDR1, CDR2, and CDR3 sequences of SEQ ID NOs: 136, 146, and 138, respectively, and the VL of the scFv comprises CDR1, CDR2, and CDR3 sequences of SEQ ID NOs: 140, 141, and 151, respectively; and
(c) the antibody Fc domain comprises a first antibody Fc polypeptide linked to the Fab and a second antibody Fc polypeptide linked to the scFv, wherein the first antibody Fc polypeptide is a human IgG1 Fc polypeptide comprising K360E and K409W substitutions, and the second antibody Fc polypeptide is a human IgG1 Fc polypeptide comprising Q347R, D399V, and F405T substitutions, numbered according to the EU index.

520. The method of any one of claims 504 to 519, wherein the multi-specific binding protein comprises:
(a) a first polypeptide comprising the amino acid sequence of SEQ ID NO:167;
(b) a second polypeptide comprising the amino acid sequence of SEQ ID NO:164; and
(c) a third polypeptide comprising the amino acid sequence of SEQ ID NO:165.
